(19) United States
(12) Patent Application Publication
(10) Pub. No.: US 2026/0347338 A1
(43) Pub. Date: Nov. 5, 2026

(54) PYRIMIDINE/PYRIDINE DERIVATIVES OR SALTS THEREOF AND PHARMACEUTICAL COMPOSITIONS COMPRISING THE SAME

(71) Applicant: YUHAN CORPORATION, Seoul (KR)

(72) Inventors: Jaewon JEONG, Hwaseong-si, Gyeonggi-do (KR); Eun Hye JUNG, Yongin-si, Gyeonggi-do (KR); Soyeon LEE, Suwon-si, Gyeonggi-do (KR); Hyokjun CHO, Hwaseong-si, Gyeonggi-do (KR); Kwan Hoon HYUN, Incheon (KR); Suhwa KIM, Yongin-si, Gyeonggi-do (KR); Wonee CHONG, Hwaseong-si, Gyeonggi-do (KR); Tae Kyun KIM, Hwaseong-si, Gyeonggi-do (KR)

(73) Assignee: YUHAN CORPORATION, Seoul (KR)

(21) Appl. No.: 19/491,174

(22) PCT Filed: Jun. 5, 2024
(86) PCT No.: PCT/KR2024/007677

§ 371 Date: Dec. 8, 2025

(30) Foreign Application Priority Data
Jun. 9, 2023 (KR) 10-2023-0073928

(51) Int. Cl. C07D 401/14; A61K 31/506; A61K 31/5377; C07D 405/14

(52) U.S. Cl. CPC C07D 401/14; A61K 31/506; A61K 31/5377; C07D 405/14

(57) ABSTRACT

Provided is a novel pyrimidine/pyridine derivative or pharmaceutically acceptable salt thereof, a process for the preparation thereof, a pharmaceutical composition including the same and a use thereof. The pyrimidine/pyridine derivative or pharmaceutically acceptable salt thereof can be usefully applied for preventing or treating various blood cancers or solid tumors through excellent inhibitory activity against hematopoietic progenitor kinase 1 (HPK1).

### TECHNICAL FIELD

The present invention relates to a pyrimidine/pyridine derivative or pharmaceutically acceptable salt thereof having an excellent inhibitory activity against hematopoietic progenitor kinase 1 (HPK1), a process for the preparation thereof, a pharmaceutical composition comprising the same, and a use thereof.

### BACKGROUND ART

Antibody-based cancer immunotherapy shows continuous anti-tumor effects in a significant proportion of cancer patients and thus the development of cancer immunotherapy will be one of the effective anti-cancer platforms in the future. Antibody-based cancer immunotherapy has been shown to improve survival rates compared to conventional standard therapies. However, the response rate thereof varies widely depending on tumor types, ranging from 15% in head and neck cancer patients to 87% in leukemia patients (Science 2018; 23; 359(6382):1350-1355). Such a limitation makes it difficult to utilize cancer immunotherapy as a broad-spectrum anticancer therapy and requires developing the biomarkers that can predict responsiveness (Nature 2017; 18; 541(7637):321-330; Nat. Rev. Cancer 2019; March; 19(3):133-150). Therefore, in addition to the cancer immunotherapy, there is an urgent need for alternative approaches that modulate the response of immune cells.

Recently, there are being re-examined approaches for targeting the intracellular pathway that regulates immune function by small molecules. For increasing the efficacy of cancer immunotherapy based on said approaches, it is required to control immunosuppression in the tumor microenvironment and peripheral tolerance to tumor antigens. In addition, since most of cancer immunotherapies are based on the antibody and target the receptors present on the surface of cell membranes, it is difficult to develop antibody-based cancer immunotherapy that can act on the targets present within tumor cells or immune cells.

Hematopoietic progenitor kinase 1 (HPK1), a member of the MAP4K family, is a serine/threonine kinase and is mainly expressed only in the hematopoietic cell lineage (T cells, B cells, DC cells, mononuclear cells, NK cells, etc.). HPK1 involves in various signaling mechanisms such as JNK, AP-1, and NK-κB to regulate the function of immune cells (Genes Devel. 1996; 10:2251-2264, EMBO J. 1996; 15:7013-7025, Proc Nat Acad Sci USA. 2009; 106(34):14508-14513). For example, HPK1 plays a role in suppressing excessive activity of T cells through phosphorylation and activation of the T cell receptor adapter protein SLP-76 when the T cell receptor is activated by binding to a ligand (J. Exp. Med. 2007; 204:681). In addition, HPK1 inhibits the activity of B cells by interfering with signaling of the B cell receptor through phosphorylation of BLNK protein (J. Biol. Chem. 2012; 287:11037). Therefore, increase in proliferation of T cells and increase in inflammatory cytokines can be observed in HPK1 KO/KD mice in which the function of HPK1 is suppressed. And, decrease in tumor growth rate and increase in efficacy of cancer immunotherapy can be also observed in a tumor model using HPK1 KO/KD mice (Cell Rep. 2018; 25(1):80-94). In addition, it has been reported that the activity of COX-2 and PGE2/adenosine is an important cause of reducing the efficacy of immunotherapy in the tumor microenvironment; and that tumor growth rate according to the immune response is decreased in HPK1 KD mice, even in the presence of high level of PGE2 (Cancer Immunol Immunother. 2010; 59(3):419-429). These mechanisms mean that, when HPK1 is inhibited in most of tumor patients, the activity of immune cells increases and the immunosuppression in the tumor microenvironment can also be improved. Therefore, the materials that inhibit HPK1 can be usefully applied to the prevention and treatment of any types of carcinomas, including various blood cancers and solid tumors.

### DISCLOSURE OF INVENTION

### Technical Problem

The present inventors carried out various studies to develop a material having an excellent inhibitory activity against HPK1. As a result, the present inventors have found that a pyrimidine/pyridine derivative or pharmaceutically acceptable salt thereof has an excellent inhibitory activity against HPK1. Therefore, the pyrimidine/pyridine derivative or pharmaceutically acceptable salt thereof inhibits HPK1 to activate immune cells and improve the tumor microenvironment in which the immune system is suppressed, thereby being able to be usefully applied to the prevention and treatment of various blood cancers and solid tumors.

Therefore, the present invention provides said pyrimidine/pyridine derivative or pharmaceutically acceptable salt thereof, a process for the preparation thereof, a pharmaceutical composition comprising the same, and a use thereof.

### Solution to Problem

According to an aspect of the present invention, there is provided a pyrimidine/pyridine derivative or pharmaceutically acceptable salt thereof.

According to another aspect of the present invention, there is provided a process for preparing said pyrimidine/pyridine derivative or pharmaceutically acceptable salt thereof.

According to still another aspect of the present invention, there is provided a pharmaceutical composition comprising said pyrimidine/pyridine derivative or pharmaceutically acceptable salt thereof as an active ingredient.

According to still another aspect of the present invention, there is provided a therapeutic method comprising administering said pyrimidine/pyridine derivative or pharmaceutically acceptable salt thereof.

According to still another aspect of the present invention, there is provided a use of said pyrimidine/pyridine derivative or pharmaceutically acceptable salt thereof for the manufacture of a medicament for inhibiting hematopoietic progenitor kinase 1.

### Advantageous Effects of Invention

It has been found by the present invention that the pyrimidine/pyridine derivative or pharmaceutically acceptable salt thereof has an excellent inhibitory activity against HPK1. Therefore, the compound or pharmaceutically acceptable salt thereof according to the present invention can be usefully applied for preventing or treating various blood cancers and solid tumors, through inhibiting the activity of HPK1.

### BEST MODE FOR CARRYING OUT THE INVENTION

As used herein, the term ‘alkyl’ refers to an aliphatic hydrocarbon radical, including a straight or branched aliphatic hydrocarbon radical. For example, the C₁~C₆ alkyl means aliphatic hydrocarbon having 1 to 6 carbon atoms, including methyl, ethyl, propyl, n-butyl, n-pentyl, n-hexyl, isopropyl, isobutyl, sec-butyl, tert-butyl, neopentyl, and isopentyl.

The term ‘hydroxy’ refers to the —OH group. The term ‘alkoxy’ refers to a radical formed by substituting the hydrogen atom in the hydroxyl group with an alkyl, unless otherwise defined. For example, the C₁~C₆ alkoxy includes methoxy, ethoxy, propoxy, n-butoxy, n-pentyloxy, isopropoxy, sec-butoxy, tert-butoxy, neopentyloxy, and isopentyloxy.

The term ‘halogen’ refers to the fluoro, bromo, chloro, or iodo group.

The term ‘amino’ refers to the —NH₂ group. The term ‘alkylamino’ refers to an amino group substituted with mono- or di-alkyl. For example, the C₁~C₆ alkylamino includes an amino group substituted with mono- or di- C₁~C₆ alkyl.

The term ‘carbonyl’ refers to —C(═O)—. The term ‘aminocarbonyl’ refers to a carbonyl group substituted with amino. The term ‘alkylcarbonyl’ refers to a carbonyl group substituted with alkyl. The term ‘hydroxycarbonyl’ refers to a carbonyl group substituted with hydroxy. The term ‘alkoxycarbonyl’ refers to a carbonyl group substituted with alkoxy. The term ‘heterocycloalkylcarbonyl’ refers to a carbonyl group substituted with heterocycloalkyl.

The term ‘sulfonyl’ refers to —SO₂—. The term ‘alkylsulfonyl’ refers to a sulfonyl group substituted with alkyl.

The term ‘cyano’ refers to the —CN.

The term ‘cycloalkyl’ refers to a cyclic alkyl group, where the ‘alkyl’ is the same as defined in the above. For example, the cycloalkyl includes cyclopropyl, cyclobutyl, cyclopentyl, cyclohexyl, etc., but not limited thereto.

The term ‘heterocycloalkyl’ refers to a 3- to 12-membered cycloalkyl or spirocycloalkyl group having one to three heteroatoms selected from N, O and S. For example, the heterocycloalkyl includes azetidinyl, pyrrolidinyl, piperidinyl, N-methylpiperidinyl, pyrazolidinyl, morpholinyl, piperazinyl, tetrahydropyranyl, tetrahydrofuranyl, oxazolidin-2-onyl, 2,8-diazaspiro[4.5]decanyl, etc., but not limited thereto.

The term ‘aryl’ refers to a 3- to 12-membered aromatic ring and may be a single ring or multiple rings. For examples, the aryl includes phenyl, naphthyl, biphenyl, etc., but not limited thereto.

The term ‘heteroaryl’ refers to a 3- to 12-membered aromatic ring having one to three heteroatoms selected from N, O and S and may be a single ring or multiple rings. For example, the heteroaryl includes thienyl, furyl, pyrrolyl, pyrazolyl, thiazolyl, oxazolyl, pyridinyl, pyrimidinyl, quinolinyl, quinazolinyl, quinoxalinyl, [1,2,4]triazolo[1,5-a]pyridinyl, pyrazolo[1,5-a]pyridinyl, imidazo[1,2-a]pyridinyl, [1,2,4]triazolo[4,3-a]pyridin-6-yl, etc., but not limited thereto.

The term ‘substituted’ refers to being substituted at one or more substitutable positions. For example, the expression ‘C₁~C₆ alkyl substituted with halogen’ includes a C₁~C₆ alkyl group substituted with one or more halogens, for example, 1, 2 or 3 halogens.

The present invention provides a pyrimidine/pyridine derivative or pharmaceutically acceptable salt thereof having an excellent inhibitory activity against HPK1, i.e., the following compound of Formula 1 or pharmaceutically acceptable salt thereof:

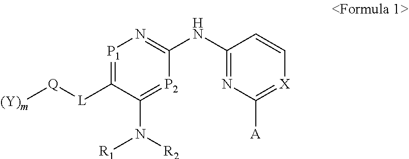

wherein,
P₁ and P₂ are, independently each other, —CH═ or —N═,
R₁ and R₂ are, independently each other, a C₁~C₆ alkyl group; a C₃~C₆ cycloalkyl group optionally substituted with hydroxy or amino; or a heterocycloalkyl optionally substituted with hydroxy or amino, or
R₁ and R₂ form a heterocycloalkyl together with the nitrogen atom to which they are bonded, wherein the heterocycloalkyl is optionally substituted with one or two groups selected from the group consisting of halogen, amino, hydroxy, C₁~C₆ alkyl, C₁~C₆ alkoxy, C₁~C₆ alkyl substituted with halogen, C₁~C₆ alkyl substituted with hydroxy, C₃~C₆ cycloalkyl, C₃~C₆ cycloalkyl substituted with hydroxy, mono- or di-C₁~C₆ alkylamino, aminocarbonyl, C₁~C₆ alkylcarbonylamino, and mono- or di-C₁~C₆ alkylamino-C₁~-C₆ alkyl,
X is —C(R₃)═ or —N═,
R₃ is hydrogen, halogen, cyano, nitro, amino, C₁~C₃ alkyl, C₁~C₃ alkyl substituted with halogen, hydroxycarbonyl, C₁~C₃ alkoxycarbonyl, aminocarbonyl, mono- or di-C₁~C₃ alkylaminocarbonyl, or C₁~C₃ alkylsulfonyl,

A is a 5- to 12-membered aryl group; a 5- to 12-membered heteroaryl group having one to three heteroatoms selected from N, O and S; or a 5- to 12-membered heterocycloalkyl group having one to three heteroatoms selected from N, O and S, wherein the aryl group, the heteroaryl group, or the heterocycloalkyl group is optionally substituted with one to three groups selected from the group consisting of halogen, amino, hydroxy, C₁~C₆ alkyl, C₁~C₆ alkyl substituted with hydroxy, C₁~C₆ alkoxy, C₃~C₆ cycloalkyl, aldehyde, mono- or di-C₁~C₃ alkylamino, mono- or di-C₁~C₃ alkylamino-C₁~C₆ alkyl, C₁~-C₆ alkoxycarbonyl, mono- or di-C₁~-C₆ alkylaminocarbonyl, C₁~C₆ alkylaminocarbonyl substituted with halogen, C₃~C₆ cycloalkylaminocarbonyl, C₃~C₆ cycloalkylaminocarbonyl substituted with halogen, C₃~C₆ cycloalkylaminocarbonyl substituted with hydroxy, heterocycloalkyl-C₁~-C₃ alkyl optionally substituted with hydroxy, heterocycloalkylcarbonyl optionally substituted with halogen, and oxo,
L is a bond, —C(O)—, —(CH₂)ₙ—, —C(O)NH—, or —C≡C—,
n is 1, 2, or 3,
Q is a bond; C₃~C₆ cycloalkyl; a 3- to 12-membered heterocycloalkyl having one to three heteroatoms selected from N, O and S; a 5- to 12-membered heteroaryl having one to three heteroatoms selected from N, O and S; or a 5- to 12-membered aryl,
m is 1, 2, or 3,
Y is, independently each other, hydrogen, hydroxy, halogen, amino, C₁~-C₆ alkyl, C₁~C₆ alkyl substituted with hydroxy, C₁~-C₆ alkyl substituted with halogen, C₁~C₆ alkyl substituted with mono- or di-C₁~C₃ alkylamino, C₁~C₆ alkoxy, C₁~C₆ alkoxy substituted with C₁~C₃ alkoxy, C₁~C₆ alkoxy substituted with halogen, C₁~C₆ alkoxy substituted with mono- or di-C₁~C₃ alkylamino, C₁~C₆ alkoxy-C₁~C₆ alkyl, C₃~C₆ cycloalkyl, C₃~C₆ cycloalkyl-C₁~C₃ alkyl, C₃~C₆ cycloalkyl-C₁~C₃ alkyl substituted with halogen, C₁~C₃ alkylcarbonyl, C₃~C₆ cycloalkyl-aminocarbonyl substituted with hydroxy, mono- or di-C₁~C₃ alkylamino, C₁~C₆ alkylamino substituted with mono- or di-C₁~C₃ alkylamino, aminocarbonyl-C₁~C₆ alkyl, C₁~C₃ alkoxycarbonyl-C₁~C₆ alkyl, C₁~C₃ alkylcarbonylamino, mono- or di-C₁~C₆ alkylaminocarbonyl, mono- or di-C₁~C₆ alkylaminocarbonylamino, or mono- or di-C₁~C₆ alkylaminocarbonyl-C₁~C₆ alkyl, or
a heterocycloalkyl-containing group selected from the group consisting of azetidinyl; azetidinyl-C₁~C₆ alkyl; tetrahydropyranyl; tetrahydrofuranyl; pyrrolidinyl; pyrrolidinyl-C₁~C₃ alkyl; piperidinyl; piperidinyl-C₁~C₃ alkyl; piperidinylcarbonyl; piperidinyloxy; piperazinyl; piperazinyl-C₁~C₃ alkyl; piperazinylcarbonyl; oxetanyl; morpholinyl; morpholinyl-C₁~C₃ alkyl; and morpholinylcarbonyl, wherein the heterocycloalkyl of the heterocycloalkyl-containing group is optionally substituted with one to four groups selected from the group consisting of halogen, hydroxy, amino, oxo, C₁~C₃ alkyl, C₁~C₃ alkyl substituted with hydroxy, and mono- or di-C₁~C₃ alkylamino.

In an embodiment of the present invention, P₁ may be —CH═ and P₂ may be —CH═ or —N═. In another embodiment of the present invention, P₁ may be —N═ and P₂ may be —CH═.

In the compound of Formula 1 or pharmaceutically acceptable salt thereof according to the present invention, R₁ and R₂ may form piperidine, azetidine, pyrrolidine, piperazine, or 2,8-diazaspiro[4.5]decane together with the nitrogen atom to which they are bonded. In an embodiment, R₁ may be a C₁~C₆ alkyl group and R₂ may be a C₃~C₆ cycloalkyl group substituted with hydroxy.

In an embodiment of the present invention, X may be —N═.

In another embodiment of the present invention, X may be —C(R₃)═ and R₃ may be H, —F, —Cl, —CH₃, —CN, —NO₂, —NH₂, —C(O)OH, —C(O)OCH₃, —C(O)NH₂, —C(O)N(CH₃)₂, or —SO₂CH₃.

In the compound of Formula 1 or pharmaceutically acceptable salt thereof according to the present invention, A may be preferably phenyl, pyridinyl, thienyl, [1,2,4]triazolo[1,5-a]pyridinyl, pyrazolo[1,5-a]pyridinyl, quinolinyl, quinoxalinyl, or oxazolidinyl. In an embodiment, A may be optionally substituted with one to three groups selected from the group consisting of halogen, amino, hydroxy, C₁~C₆ alkyl, C₁~-C₆ alkyl substituted with hydroxy, C₁~-C₆ alkoxy, aldehyde, mono- or di-C₁~C₃ alkylamino-C₁~C₆ alkyl, C₁~C₆ alkoxycarbonyl, mono- or di-C₁-C₆ alkylaminocarbonyl, azetidinyl-C₁-C₃ alkyl substituted with hydroxy, azetidinylcarbonyl optionally substituted with one or two halogens, pyrrolidinylcarbonyl optionally substituted with one or two halogens, and oxo,

In the compound of Formula 1 or pharmaceutically acceptable salt thereof according to the present invention, A may be particularly preferably phenyl. That is, particularly preferably, the present invention includes a compound of Formula 1a or pharmaceutically acceptable salt thereof:

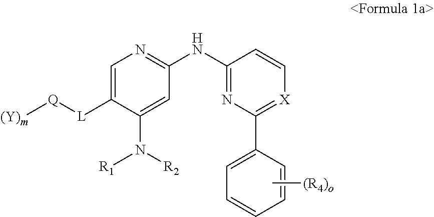

wherein,
R₁ and R₂ form a heterocycloalkyl together with the nitrogen atom to which they are bonded, wherein the heterocycloalkyl is optionally substituted with one or two groups selected from the group consisting of halogen, amino, hydroxy, C₁~C₆ alkyl, C₁~C₆ alkoxy, C₁~C₆ alkyl substituted with halogen, C₁~C₆ alkyl substituted with hydroxy, C₃~C₆ cycloalkyl, C₃~C₆ cycloalkyl substituted with hydroxy, mono- or di-C₁~C₆ alkylamino, aminocarbonyl, C₁~C₆ alkylcarbonylamino, and mono- or di-C₁~-C₆ alkylamino-C₁~-C₆ alkyl,
X is —C(R₃)═ or —N═,
R₃ is hydrogen, halogen, cyano, nitro, amino, C₁~C₃ alkyl, C₁~C₃ alkyl substituted with halogen, hydroxycarbonyl, C₁~C₃ alkoxycarbonyl, aminocarbonyl, mono- or di-C₁~C₃ alkylaminocarbonyl, or C₁~C₃ alkylsulfonyl,
R₄ is, independently each other, halogen, amino, hydroxy, C₁~-C₆ alkyl, C₁~-C₆ alkyl substituted with hydroxy, C₁~C₆ alkoxy, C₃~C₆ cycloalkyl, aldehyde, mono- or di-C₁~C₃ alkylamino, mono- or di-C₁~C₃ alkylamino-C₁~C₆ alkyl, C₁~C₆ alkoxycarbonyl, mono- or di-C₁~C₆ alkylaminocarbonyl, C₁~C₆ alkylaminocarbonyl substituted with halogen, C₃~C₆ cycloalkylaminocarbonyl, C₃~C₆ cycloalkylaminocarbonyl substituted with halogen, C₃~C₆ cycloalkylaminocarbonyl substituted with hydroxy, heterocycloalkyl-C₁~C₃ alkyl optionally substituted with hydroxy, or heterocycloalkylcarbonyl optionally substituted with halogen,
o is 0, 1, 2, or 3,
L is a bond, —C(O)—, —(CH₂)ₙ—, —C(O)NH—, or —C≡C—,
n is 1, 2, or 3,
Q is a bond; C₃~C₆ cycloalkyl; a 3- to 12-membered heterocycloalkyl having one to three heteroatoms selected from N, O and S; a 5- to 12-membered heteroaryl having one to three heteroatoms selected from N, O and S; or a 5- to 12-membered aryl,
m is 1, 2, or 3,
Y is, independently each other, hydrogen, hydroxy, halogen, amino, C₁~C₆ alkyl, C₁~C₆ alkyl substituted with hydroxy, C₁~C₆ alkyl substituted with halogen, C₁~C₆ alkyl substituted with mono- or di-C₁~C₃ alkylamino, C₁~C₆ alkoxy, C₁~C₆ alkoxy substituted with C₁~C₃ alkoxy, C₁~C₆ alkoxy substituted with halogen, C₁~C₆ alkoxy substituted with mono- or di-C₁~C₃ alkylamino, C₁~C₆ alkoxy-C₁~C₆ alkyl, C₃~C₆ cycloalkyl, C₃~C₆ cycloalkyl-C₁~C₃ alkyl, C₃~C₆ cycloalkyl-C₁~C₃ alkyl substituted with halogen, C₁~C₃ alkylcarbonyl, C₃~C₆ cycloalkyl-aminocarbonyl substituted with hydroxy, mono- or di-C₁~C₃ alkylamino, C₁~C₆ alkylamino substituted with mono- or di-C₁~C₃ alkylamino, aminocarbonyl-C₁~C₆ alkyl, C₁~C₃ alkoxycarbonyl-C₁~C₆ alkyl, C₁~C₃ alkylcarbonylamino, mono- or di-C₁~C₆ alkylaminocarbonyl, mono- or di-C₁~C₆ alkylaminocarbonylamino, or mono- or di-C₁~C₆ alkylaminocarbonyl-C₁~C₆ alkyl, or
a heterocycloalkyl-containing group selected from the group consisting of azetidinyl; azetidinyl-C₁~C₆ alkyl; tetrahydropyranyl; tetrahydrofuranyl; pyrrolidinyl; pyrrolidinyl-C₁~C₃ alkyl; piperidinyl; piperidinyl-C₁~C₃ alkyl; piperidinylcarbonyl; piperidinyloxy; piperazinyl; piperazinyl-C₁~C₃ alkyl; piperazinylcarbonyl; oxetanyl; morpholinyl; morpholinyl-C₁~C₃ alkyl; and morpholinylcarbonyl, wherein the heterocycloalkyl of the heterocycloalkyl-containing group is optionally substituted with one to four groups selected from the group consisting of halogen, hydroxy, amino, oxo, C₁~C₃ alkyl, C₁~C₃ alkyl substituted with hydroxy, and mono- or di-C₁~C₃ alkylamino.

In the compound of Formula 1a or pharmaceutically acceptable salt thereof, R₁ and R₂ may form preferably piperidine, azetidine, pyrrolidine, piperazine, or 2,8-diazaspiro[4.5]decane together with the nitrogen atom to which they are bonded.

In the compound of Formula 1a or pharmaceutically acceptable salt thereof, X may be preferably —N═.

In the compound of Formula 1a or pharmaceutically acceptable salt thereof, X may be —C(R₃)═ and R₃ may be preferably H, —F, —Cl, —CH₃, —CN, —NO₂, —NH₂, —C(O)OH, —C(O)OCH₃, —C(O)NH₂, —C(O)N(CH₃)₂, or —SO₂CH₃.

In the compound of Formula 1a or pharmaceutically acceptable salt thereof, R₄ may be, independently each other, preferably halogen, amino, hydroxy, C₁~C₆ alkyl, C₁~C₆ alkyl substituted with hydroxy, C₁~C₆ alkoxy, aldehyde, mono- or di-C₁~C₃ alkylamino-C₁~C₆ alkyl, C₁~C₆ alkoxycarbonyl, mono- or di-C₁-C₆ alkylaminocarbonyl, azetidinyl-C₁~C₃ alkyl substituted with hydroxy, azetidinylcarbonyl optionally substituted with one or two halogens, or pyrrolidinylcarbonyl optionally substituted with one or two halogens.

In the compound of Formula 1 (preferably Formula 1a) or pharmaceutically acceptable salt thereof of the present invention, when Q is a ring such as C₃~C₆ cycloalkyl; a 3- to 12-membered heterocycloalkyl having one to three heteroatoms selected from N, O and S; a 5- to 12-membered heteroaryl having one to three heteroatoms selected from N, O and S; or a 5- to 12-membered aryl, Y may be substituted at any substitutable position(s) within the ring.

In the compound of Formula 1 (preferably Formula 1a) or pharmaceutically acceptable salt thereof of the present invention, Q may be a bond, C₃~C₆ cycloalkyl, tetrahydropyranyl, pyrrolidinyl, phenyl, pyridinyl, pyrazolyl, thiazolyl, imidazo[1,2-a]pyridinyl, [1,2,4]triazolo[4,3-a]pyridin-6-yl, 2H-benzo[b][1,4]oxazin-3(4H)-onyl, 3,4-dihydroquinolin-2(1H)-onyl, 3,4-dihydro-2H-benzo[b][1,4]oxazinyl, 3,4-dihydro-2H-pyrido[3,2-b][1,4]oxazinyl, 2H-pyrido[3,2-b]-1,4-oxazin-3(4H)-onyl, 1,4-dihydro-2H-benzo[d][1,3]oxazin-2-onyl, or 3,4-dihydro-1,8-naphthyridin-2(1H)-onyl.

In the compound of Formula 1 (preferably Formula 1a) or pharmaceutically acceptable salt thereof of the present invention, Y may be, independently each other, hydrogen, hydroxy, halogen, amino, C₁~C₆ alkyl, C₁~C₆ alkyl substituted with hydroxy, C₁~C₆ alkyl substituted with halogen, C₁~C₆ alkyl substituted with mono- or di-C₁~C₃ alkylamino, C₁~C₆ alkoxy, C₁~C₆ alkoxy substituted with C₁~C₃ alkoxy, C₁~C₆ alkoxy substituted with halogen, C₁~C₆ alkoxy substituted with mono- or di-C₁~C₃ alkylamino, C₁~C₆ alkoxy-C₁~C₆ alkyl, C₃~C₆ cycloalkyl, C₃~C₆ cycloalkyl-C₁~C₃ alkyl, C₃~C₆ cycloalkyl-C₁~C₃ alkyl substituted with halogen, C₁~C₃ alkylcarbonyl, C₃~C₆ cycloalkyl-aminocarbonyl substituted with hydroxy, mono- or di-C₁~C₃ alkylamino, C₁~C₆ alkylamino substituted with mono- or di-C₁~C₃ alkylamino, aminocarbonyl-C₁~C₆ alkyl, C₁~C₃ alkoxycarbonyl-C₁~C₆ alkyl, C₁~C₃ alkylcarbonylamino, mono- or di-C₁~C₆ alkylaminocarbonyl, mono- or di-C₁~C₆ alkylaminocarbonylamino, or mono- or di-C₁~C₆ alkylaminocarbonyl-C₁~C₆ alkyl.

In the compound of Formula 1 (preferably Formula 1a) or pharmaceutically acceptable salt thereof of the present invention, one of Y may be a heterocycloalkyl-containing group selected from the group consisting of azetidinyl; azetidinylmethyl; tetrahydropyranyl; tetrahydrofuranyl; pyrrolidinyl; pyrrolidinylmethyl; pyrrolidinylethyl; piperidinyl; piperidinylmethyl; piperidinylcarbonyl; piperidinyloxy; piperazinyl; piperazinylmethyl; piperazinylcarbonyl; oxetanyl; morpholinyl; morpholinylmethyl; morpholinylethyl; and morpholinylcarbonyl, and the remaining Y may be hydrogen. In addition, the heterocycloalkyl of the heterocycloalkyl-containing group may be substituted with one to four groups selected from the group consisting of halogen, hydroxy, amino, oxo, C₁~C₃ alkyl, C₁~C₃ alkyl substituted with hydroxy, and mono- or di-C₁~C₃ alkylamino.

In the compound of Formula 1 or pharmaceutically acceptable salt, preferable compounds include the following compound or pharmaceutically acceptable salt thereof selected from the group consisting of:
(S)-cyclopropyl(4-(3-hydroxypiperidin-1-yl)-6-((2-(5-methylpyridin-3-yl)pyrimidin-4-yl)amino)pyridin-3-yl)methanone;
(S)-(6-((2-(5-aminopyridin-3-yl)pyrimidin-4-yl)amino)-4-(3-hydroxypiperidin-1-yl)pyridin-3-yl)(cyclopropyl)methanone;
(S)-cyclopropyl(4-(3-hydroxypiperidin-1-yl)-6-((2-(5-methoxypyridin-3-yl)pyrimidin-4-yl)amino)pyridin-3-yl)methanone;
(S)-cyclopropyl(6-((2-(3-hydroxyphenyl)pyrimidin-4-yl)amino)-4-(3-hydroxypiperidin-1-yl)pyridin-3-yl)methanone;
(S)-cyclopropyl(6-((2-(2-fluoro-6-methoxyphenyl)pyrimidin-4-yl)amino)-4-(3-hydroxypiperidin-1-yl)pyridin-3-yl)methanone;
cyclopropyl(6-((2-(2-fluoro-6-methoxyphenyl)pyrimidin-4-yl)amino)-4-(3-hydroxyazetidin-1-yl)pyridin-3-yl)methanone;
(S)-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-ol;
(S)-1-(2-((2-(2-fluoro-6-methoxyphenyl)pyrimidin-4-yl)amino)-5-((1-(trifluoromethyl)-1H-pyrazol-4-yl)ethynyl)pyridin-4-yl)piperidin-3-ol;
(R)-1-(2-((2-(2-fluoro-6-methoxyphenyl)pyrimidin-4-yl)amino)-5-((1-(trifluoromethyl)-1H-pyrazol-4-yl)ethynyl)pyridin-4-yl)piperidin-3-ol;
1-(2-((2-(2-fluoro-6-methoxyphenyl)pyrimidin-4-yl)amino)-5-((tetrahydro-2H-pyran-4-yl)ethynyl)pyridin-4-yl)azetidin-3-ol;
(S)-1-(2-((2-(2-fluoro-6-methoxyphenyl)pyrimidin-4-yl)amino)-5-((tetrahydro-2H-pyran-4-yl)ethynyl)pyridin-4-yl)piperidin-3-ol;
(S)-1-(2-((2-(2-fluoro-6-methoxyphenyl)pyrimidin-4-yl)amino)-5-(1-methyl-1H-pyrazol-4-yl)pyridin-4-yl)piperidin-3-ol;
(S)-1-(2-((2-(2-fluoro-6-methoxyphenyl)pyrimidin-4-yl)amino)-5-(1-isopropyl-1H-pyrazol-4-yl)pyridin-4-yl)piperidin-3-ol;
(3S)-1-(2-((2-(2-fluoro-6-methoxyphenyl)pyrimidin-4-yl)amino)-5-(1-(tetrahydrofuran-3-yl)-1H-pyrazol-4-yl)pyridin-4-yl)piperidin-3-ol;
(S)-1-(2-((2-(2-fluoro-6-methoxyphenyl)pyrimidin-4-yl)amino)-5-(1-(1-methylpiperidin-4-yl)-1H-pyrazol-4-yl)pyridin-4-yl)piperidin-3-ol;
(S)-1-(2-((2-(2-fluoro-6-methoxyphenyl)pyrimidin-4-yl)amino)-5-(1-(2,2,2-trifluoroethyl)-1H-pyrazol-4-yl)pyridin-4-yl)piperidin-3-ol;
(S)-1-(5-(1-(cyclopropylmethyl)-1H-pyrazol-4-yl)-2-((2-(2-fluoro-6-methoxyphenyl)pyrimidin-4-yl)amino)pyridin-4-yl)piperidin-3-ol;
(S)-1-(2-((2-(2-fluoro-6-methoxyphenyl)pyrimidin-4-yl)amino)-5-(4-(tetrahydro-2H-pyran-4-yl)phenyl)pyridin-4-yl)piperidin-3-ol;
(S)-1-(2-((2-(2-fluoro-6-methoxyphenyl)pyrimidin-4-yl)amino)-5-(4-(4-methylpiperazin-1-yl)phenyl)pyridin-4-yl)piperidin-3-ol;
(S)-1-(6-((2-(2-fluoro-6-methoxyphenyl)pyrimidin-4-yl)amino)-6′-morpholino-[3,3′-bipyridin]-4-yl)piperidin-3-ol;
(S)-1-(2-((2-(4-(hydroxymethyl)thiophen-2-yl)pyrimidin-4-yl)amino)-5-(1-(tetrahydro-2H-pyran-4-yl)-1H-pyrazol-4-yl)pyridin-4-yl)piperidin-3-ol;
(S)-1-(2-((2-(2,6-difluorophenyl)pyrimidin-4-yl)amino)-5-(1-(tetrahydro-2H-pyran-4-yl)-1H-pyrazol-4-yl)pyridin-4-yl)piperidin-3-ol;
(S)-1-(2-((2-(3,6-difluoro-2-methoxyphenyl)pyrimidin-4-yl)amino)-5-(1-(tetrahydro-2H-pyran-4-yl)-1H-pyrazol-4-yl)pyridin-4-yl)piperidin-3-ol;
(S)-1-(2-((2-(2,3-difluoro-6-methoxyphenyl)pyrimidin-4-yl)amino)-5-(1-(tetrahydro-2H-pyran-4-yl)-1H-pyrazol-4-yl)pyridin-4-yl)piperidin-3-ol;
(S)-1-(2-((2-(2-ethoxy-6-fluorophenyl)pyrimidin-4-yl)amino)-5-(1-(tetrahydro-2H-pyran-4-yl)-1H-pyrazol-4-yl)pyridin-4-yl)piperidin-3-ol;
(S)-1-(2-((2-([1,2,4]triazolo[1,5-a]pyridin-7-yl)pyrimidin-4-yl)amino)-5-(1-(tetrahydro-2H-pyran-4-yl)-1H-pyrazol-4-yl)pyridin-4-yl)piperidin-3-ol;
(S)-1-(2-((2-(pyrazolo[1,5-a]pyridin-3-yl)pyrimidin-4-yl)amino)-5-(1-(tetrahydro-2H-pyran-4-yl)-1H-pyrazol-4-yl)pyridin-4-yl)piperidin-3-ol;
(S)-1-(2-((2-(quinoxalin-6-yl)pyrimidin-4-yl)amino)-5-(1-(tetrahydro-2H-pyran-4-yl)-1H-pyrazol-4-yl)pyridin-4-yl)piperidin-3-ol;
(S)-1-(2-((2-(8-fluoroquinolin-7-yl)pyrimidin-4-yl)amino)-5-(1-(tetrahydro-2H-pyran-4-yl)-1H-pyrazol-4-yl)pyridin-4-yl)piperidin-3-ol;
(S)-1-(6-((2-(2-fluoro-6-methoxyphenyl)pyrimidin-4-yl)amino)-6′-((4-methylpiperazin-1-yl)methyl)-[3,3′-bipyridin]-4-yl)piperidin-3-ol;
(S)-1-(6-((2-(2-fluoro-6-methoxyphenyl)pyrimidin-4-yl)amino)-6′-(tetrahydro-2H-pyran-4-yl)-[3,3′-bipyridin]-4-yl)piperidin-3-ol;
(S)-1-(6-((2-(2-fluoro-6-methoxyphenyl)pyrimidin-4-yl)amino)-6′-(morpholinomethyl)-[3,3′-bipyridin]-4-yl)piperidin-3-ol;
(S)-1-(6-((2-(2-fluoro-6-methoxyphenyl)pyrimidin-4-yl)amino)-6′-((4-hydroxypiperidin-1-yl)methyl)-[3,3′-bipyridin]-4-yl)piperidin-3-ol;
1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-carboxamide;
(S)-1-(2-((6-(2-fluoro-6-methoxyphenyl)pyridin-2-yl)amino)-5-(1-(tetrahydro-2H-pyran-4-yl)-1H-pyrazol-4-yl)pyridin-4-yl)piperidin-3-ol;
(S)-(6′-((2-(2-fluoro-6-methoxyphenyl)pyrimidin-4-yl)amino)-4′-(3-hydroxypiperidin-1-yl)-[3,3′-bipyridin]-6-yl)(4-methylpiperazin-1-yl)methanone;
(S)-(6′-((2-(2-fluoro-6-methoxyphenyl)pyrimidin-4-yl)amino)-4′-(3-hydroxypiperidin-1-yl)-[3,3′-bipyridin]-6-yl)(morpholino)methanone;
(S)-(6′-((2-(2-fluoro-6-methoxyphenyl)pyrimidin-4-yl)amino)-4′-(3-hydroxypiperidin-1-yl)-[3,3′-bipyridin]-6-yl)(4-hydroxypiperidin-1-yl)methanone;
(S)-1-(6-((2-(2-fluoro-6-methoxyphenyl)pyrimidin-4-yl)amino)-6′-(((S)-3-fluoropyrrolidin-1-yl)methyl)-[3,3′-bipyridin]-4-yl)piperidin-3-ol;
(3S)-1-(2-((5-fluoro-6-(2-fluoro-6-methoxyphenyl)pyridin-2-yl)amino)-5-(1-(tetrahydro-2H-pyran-4-yl)-1H-pyrazol-4-yl)pyridin-4-yl)piperidin-3-ol;
(3S)-1-(2-((6-(2-fluoro-6-methoxyphenyl)-5-nitropyridin-2-yl)amino)-5-(1-(tetrahydro-2H-pyran-4-yl)-1H-pyrazol-4-yl)pyridin-4-yl)piperidin-3-ol;
(S)-1-(6-((2-(2-fluoro-6-methoxyphenyl)pyrimidin-4-yl)amino)-[3,3′-bipyridin]-4-yl)piperidin-3-ol;
(3S)-1-(6-((2-(2-fluoro-6-methoxyphenyl)pyrimidin-4-yl)amino)-6′-((3-hydroxypyrrolidin-1-yl)methyl)-[3,3′-bipyridin]-4-yl)piperidin-3-ol;
N-(4-(3-(dimethylamino)pyrrolidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)-3-methylpyrrolidin-3-yl)methanol;
N-(4-(4-((dimethylamino)methyl)-4-fluoropiperidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
(4-(2,2-difluoroethyl)-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-4-yl)methanol;
(3S)-1-(6′-((3-(dimethylamino)pyrrolidin-1-yl)methyl)-6-((2-(2-fluoro-6-methoxyphenyl)pyrimidin-4-yl)amino)-[3,3′-bipyridin]-4-yl)piperidin-3-ol;
1-((6′-((2-(2-fluoro-6-methoxyphenyl)pyrimidin-4-yl)amino)-4′-((S)-3-hydroxypiperidin-1-yl)-[3,3′-bipyridin]-6-yl)methyl)piperidin-3-ol;
(S)-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-carboxamide;
(R)-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-carboxamide;
(S)-N-(4-(3-aminopiperidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
(R)-N-(4-(3-aminopiperidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
(6′-((6-(2-fluoro-6-methoxyphenyl)-5-nitropyridin-2-yl)amino)-4′-((S)-3-hydroxypiperidin-1-yl)-[3,3′-bipyridin]-6-yl)(4-hydroxypiperidin-1-yl)methanone;
(S)-1-(6′-((4-aminopiperidin-1-yl)methyl)-6-((2-(2-fluoro-6-methoxyphenyl)pyrimidin-4-yl)amino)-1[3,3′-bipyridin]-4-yl)piperidin-3-ol;
(S)-1-(6′-ethoxy-6-((2-(2-fluoro-6-methoxyphenyl)pyrimidin-4-yl)amino)-[3,3′-bipyridin]-4-yl)piperidin-3-ol;
(S)-1-(6-((2-(2-fluoro-6-methoxyphenyl)pyrimidin-4-yl)amino)-6′-(trifluoromethyl)-[3,3′-bipyridin]-4-yl)piperidin-3-ol;
(3S)-1-(2-((6-(2-fluoro-6-methoxyphenyl)-5-methylpyridin-2-yl)amino)-5-(1-(tetrahydro-2H-pyran-4-yl)-1H-pyrazol-4-yl)pyridin-4-yl)piperidin-3-ol;
2-(2-fluoro-6-methoxyphenyl)-6-((4-((S)-3-hydroxypiperidin-1-yl)-5-(1-(tetrahydro-2H-pyran-4-yl)-1H-pyrazol-4-yl)pyridin-2-yl)amino)nicotinonitrile;
methyl 2-(2-fluoro-6-methoxyphenyl)-6-((4-((S)-3-hydroxypiperidin-1-yl)-5-(1-(tetrahydro-2H-pyran-4-yl)-1H-pyrazol-4-yl)pyridin-2-yl)amino)nicotinate;
(S)-1-(2-((2-(2-fluoro-6-methoxyphenyl)pyrimidin-4-yl)amino)-5-(imidazo[1,2-a]pyridin-6-yl)pyridin-4-yl)piperidin-3-ol;
(S)-1-(5-([1,2,4]triazolo[4,3-a]pyridin-6-yl)-2-((2-(2-fluoro-6-methoxyphenyl)pyrimidin-4-yl)amino)pyridin-4-yl)piperidin-3-ol;
2-(2-fluoro-6-methoxyphenyl)-6-((4-((S)-3-hydroxypiperidin-1-yl)-5-(1-(1-methylpiperidin-4-yl)-1H-pyrazol-4-yl)pyrimidin-2-yl)amino)nicotinonitrile;
N-(4-(4-(2-(dimethylamino)ethyl)piperidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
1-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-4-yl)cyclopropan-1-ol;
1-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-4-yl)ethan-1-ol;
2-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-4-yl)-2-methylpropan-1-ol;
2-(2-fluoro-6-methoxyphenyl)-N-(4-(4-methylpiperazin-1-yl)-5-(1-(tetrahydro-2H-pyran-4-yl)-1H-pyrazol-4-yl)pyridin-2-yl)pyrimidin-4-amine;
(S)-1-(2-((2-(7-fluoroquinoxalin-6-yl)pyrimidin-4-yl)amino)-5-(1-(tetrahydro-2H-pyran-4-yl)-1H-pyrazol-4-yl)pyridin-4-yl)piperidin-3-ol;
4-((S)-3-aminopiperidin-1-yl)-N-(6-(2-fluoro-6-methoxyphenyl)-5-nitropyridin-2-yl-) 5-(1-(tetrahydro-2H-pyran-4-yl)-1H-pyrazol-4-yl)pyridin-2-amine;
methyl 6-((4-((S)-3-aminopiperidin-1-yl)-5-(1-(tetrahydro-2H-pyran-4-yl)-1H-pyrazol-4-yl)pyridin-2-yl)amino)-2-(2-fluoro-6-methoxyphenyl)nicotinate;
(S)-1-(5-(1-(difluoromethyl)-1H-pyrazol-4-yl)-2-((2-(2-fluoro-6-methoxyphenyl)pyrimidin-4-yl)amino)pyridin-4-yl)piperidin-3-ol;
(S)-N-(4-(3-aminopiperidin-1-yl)-5-(1-(difluoromethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(1-methylpiperidin-4-yl)-1H-pyrazol-4-yl)pyridin-2-yl)-2-(2-fluoro-6-methoxyphenyl)pyrimidin-4-amine;
(3S)-1-(2-((5-chloro-6-(2-fluoro-6-methoxyphenyl)pyridin-2-yl)amino)-5-(1-(tetrahydro-2H-pyran-4-yl)-1H-pyrazol-4-yl)pyridin-4-yl)piperidin-3-ol;
4-((S)-3-aminopiperidin-1-yl)-5-(1-(difluoromethyl)-1H-pyrazol-4-yl)-N-(5-fluoro-6-(2-fluoro-6-methoxyphenyl)pyridin-2-yl)pyridin-2-amine;
(R)-N-(4-(3-aminopiperidin-1-yl)-5-(1-(2,2,2-trifluoroethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(2,2,2-trifluoroethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-((tetrahydro-2H-pyran-4-yl)ethynyl)pyridin-2-yl)-2-(2-fluoro-6-methoxyphenyl)pyrimidin-4-amine;
(S)-3-(4-((4-(3-aminopiperidin-1-yl)-5-(1-(tetrahydro-2H-pyran-4-yl)-1H-pyrazol-4-yl)pyridin-2-yl)amino)pyrimidin-2-yl)pyridin-2-ol;
(S)-1-(6′-((2-(2-fluoro-6-methoxyphenyl)pyrimidin-4-yl)amino)-5-((1-methylpiperidin-4-yl)oxy)-[2,3′-bipyridin]-4′-yl)piperidin-3-ol;
(S)-4-(3-aminopiperidin-1-yl)-N-(2-(2-fluoro-6-methoxyphenyl)pyrimidin-4-yl)-6′-(tetrahydro-2H-pyran-4-yl)-[3,3′-bipyridin]-6-amine;
(S)-4-(3-aminopiperidin-1-yl)-N-(2-(2-fluoro-6-methoxyphenyl)pyrimidin-4-yl)-6′-(1-methylpiperidin-4-yl)-[3,3′-bipyridin]-6-amine;
2-(2-fluoro-6-methoxyphenyl)-N-(5-(1-(tetrahydro-2H-pyran-4-yl)-1H-pyrazol-4-yl)-4-(3-(2,2,2-trifluoroethyl)piperidin-1-yl)pyridin-2-yl)pyrimidin-4-amine;
2-(2-fluoro-6-methoxyphenyl)-N-(4-(3-(fluoromethyl)piperidin-1-yl)-5-(1-(tetrahydro-2H-pyran-4-yl)-1H-pyrazol-4-yl)pyridin-2-yl)pyrimidin-4-amine;
N-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-yl)acetamide;
2-(2-fluoro-6-methoxyphenyl)-N-(5-(1-(tetrahydro-2H-pyran-4-yl)-1H-pyrazol-4-yl)-4-(4-(trifluoromethyl)piperidin-1-yl)pyridin-2-yl)pyrimidin-4-amine;
(S)-N-(4-(3-aminopiperidin-1-yl)-5-(1-(difluoromethyl)-1H-pyrazol-4-yl)pyridin-2-yl)-2-(7-fluoroquinoxalin-6-yl)pyrimidin-4-amine;
(S)-N-(4-(3-aminopiperidin-1-yl)-5-(1-(2,2,2-trifluoroethyl)-1H-pyrazol-4-yl)pyridin-2-yl)-2-(7-fluoroquinoxalin-6-yl)pyrimidin-4-amine;
(S)-N-(4-(3-aminopiperidin-1-yl)-5-((tetrahydro-2H-pyran-4-yl)ethynyl)pyridin-2-yl)-2-(7-fluoroquinoxalin-6-yl)pyrimidin-4-amine;
(S)-N-(4-(3-aminopiperidin-1-yl)-5-(1-(2-methoxyethyl)-1H-pyrazol-4-yl)pyridin-2-yl)-2-(2-fluoro-6-methoxyphenyl)pyrimidin-4-amine;
N-(4-(2,8-diazaspiro[4.5]decan-8-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
N-(4-(2,8-diazaspiro[4.5]decan-2-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
(3R,4R)-4-fluoro-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-ol;
(3R,4R)-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)-4-(hydroxymethyl)piperidin-3-ol;
(R)-N-(4-(3-aminopyrrolidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
(S)-N-(4-(3-aminopyrrolidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(2-(1-methylpiperidin-4-yl)thiazol-5-yl)pyridin-2-yl)-2-(2-fluoro-6-methoxyphenyl)pyrimidin-4-amine;
(S)-4-(3-aminopiperidin-1-yl)-N-(2-(7-fluoroquinoxalin-6-yl)pyrimidin-4-yl)-6′-(tetrahydro-2H-pyran-4-yl)-[3,3′-bipyridin]-6-amine;
4-((S)-3-aminopiperidin-1-yl)-N-(5-fluoro-6-(2-fluoro-6-methoxyphenyl)pyridin-2-yl)-5-(1-(2,2,2-trifluoroethyl)-1H-pyrazol-4-yl)pyridin-2-amine;
(S)-(4-(4-((4-(3-aminopiperidin-1-yl)-5-(1-(difluoromethyl)-1H-pyrazol-4-yl)pyridin-2-yl)amino)pyrimidin-2-yl)-3-fluoro-5-methoxyphenyl)methanol;
(S)-(4-(4-((4-(3-aminopiperidin-1-yl)-5-(1-(2,2,2-trifluoroethyl)-1H-pyrazol-4-yl)pyridin-2-yl)amino)pyrimidin-2-yl)-3-fluoro-5-methoxyphenyl)methanol;
(S)-(4-(4-((4-(3-aminopiperidin-1-yl)-5-((tetrahydro-2H-pyran-4-yl)ethynyl)pyridin-2-yl)amino)pyrimidin-2-yl)-3-fluoro-5-methoxyphenyl)methanol;
(S)-N-(4-(3-aminopiperidin-1-yl)-5-(1-(difluoromethyl)-1H-pyrazol-4-yl)pyridin-2-yl)-2-(4-((dimethylamino)methyl)-2-fluoro-6-methoxyphenyl)pyrimidin-4-amine;
(S)-N-(4-(3-aminopiperidin-1-yl)-5-(1-(2,2,2-trifluoroethyl)-1H-pyrazol-4-yl)pyridin-2-yl)-2-(4-((dimethylamino)methyl)-2-fluoro-6-methoxyphenyl)pyrimidin-4-amine;
(S)-N-(4-(3-aminopiperidin-1-yl)-5-((tetrahydro-2H-pyran-4-yl)ethynyl)pyridin-2-yl)-2-(4-((dimethylamino)methyl)-2-fluoro-6-methoxyphenyl)pyrimidin-4-amine;
(S)-N-(4-(3-aminopiperidin-1-yl)-5-(1-(trifluoromethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(difluoromethyl)-1H-pyrazol-4-yl)pyridin-2-yl)-2-(2,4-difluoro-6-methoxyphenyl)pyrimidin-4-amine;
(S)-N-(4-(3-aminopiperidin-1-yl)-5-(1-(2,2,2-trifluoroethyl)-1H-pyrazol-4-yl)pyridin-2-yl)-2-(2,4-difluoro-6-methoxyphenyl)pyrimidin-4-amine;
(S)-N-(4-(3-aminopiperidin-1-yl)-5-((tetrahydro-2H-pyran-4-yl)ethynyl)pyridin-2-yl)-2-(2,4-difluoro-6-methoxyphenyl)pyrimidin-4-amine;
(S)-N-(4-(3-aminopiperidin-1-yl)-5-((1-(trifluoromethyl)-1H-pyrazol-4-yl)ethynyl)pyridin-2-yl)-2-(2-fluoro-6-methoxyphenyl)pyrimidin-4-amine;
2-(2-fluoro-6-methoxyphenyl)-6-((4-((S)-3-hydroxypiperidin-1-yl)-5-(1-(tetrahydro-2H-pyran-4-yl)-1H-pyrazol-4-yl)pyridin-2-yl)amino)nicotinamide;
2-(2-fluoro-6-methoxyphenyl)-6-((4-((S)-3-hydroxypiperidin-1-yl)-5-(1-(tetrahydro-2H-pyran-4-yl)-1H-pyrazol-4-yl)pyridin-2-yl)amino)-N,N-dimethylnicotinamide;
4-((S)-3-aminopiperidin-1-yl)-N-(5-fluoro-6-(2-fluoro-6-methoxyphenyl)pyridin-2-yl)-5-((1-(trifluoromethyl)-1H-pyrazol-4-yl)ethynyl)pyridin-2-amine;
(S)-N-(4-(3-aminopiperidin-1-yl)-5-((1-(trifluoromethyl)-1H-pyrazol-4-yl)ethynyl)pyridin-2-yl)-2-(7-fluoroquinoxalin-6-yl)pyrimidin-4-amine;
(S)-N-(4-(3-aminopiperidin-1-yl)-5-(1-(3-methoxypropyl)-1H-pyrazol-4-yl)pyridin-2-yl)-2-(2-fluoro-6-methoxyphenyl)pyrimidin-4-amine;
2-(2-fluoro-6-methoxyphenyl)-N-(4-(2-methyl-2,8-diazaspiro[4.5]decan-8-yl)-5-(1-(tetrahydro-2H-pyran-4-yl)-1H-pyrazol-4-yl)pyridin-2-yl)pyrimidin-4-amine;
(S)-4-(3-aminopiperidin-1-yl)-N⁶-(2-(2-fluoro-6-methoxyphenyl)pyrimidin-4-yl)-N^{6′},N^{6′}-dimethyl-[3,3′-bipyridin]-6,6′-diamine;
(S)-4-(3-aminopiperidin-1-yl)-N-(2-(2-fluoro-6-methoxyphenyl)pyrimidin-4-yl)-6′-(pyrrolidin-1-yl)-[3,3′-bipyridin]-6-amine;
(S)-4-(3-aminopiperidin-1-yl)-N-(2-(2-fluoro-6-methoxyphenyl)pyrimidin-4-yl)-6′-(piperidin-1-yl)-[3,3′-bipyridin]-6-amine;
(S)-4-(3-aminopiperidin-1-yl)-N-(2-(2-fluoro-6-methoxyphenyl)pyrimidin-4-yl)-6′-morpholino-[3,3′-bipyridin]-6-amine;
(S)-N-(5-(1-(difluoromethyl)-1H-pyrazol-4-yl)-4-(3-(methylamino)piperidin-1-yl)pyridin-2-yl)-2-(2-fluoro-6-methoxyphenyl)pyrimidin-4-amine;
(R)-N-(5-(1-(difluoromethyl)-1H-pyrazol-4-yl)-4-(3-(methylamino)piperidin-1-yl)pyridin-2-yl)-2-(2-fluoro-6-methoxyphenyl)pyrimidin-4-amine;
(S)-2-(2-fluoro-6-methoxyphenyl)-N-(4-(3-(methylamino)pyrrolidin-1-yl)-5-(1-(tetrahydro-2H-pyran-4-yl)-1H-pyrazol-4-yl)pyridin-2-yl)pyrimidin-4-amine;
(S)-N-(4-(3-(ethylamino)pyrrolidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
(R)-N-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)pyrrolidin-3-yl)acetamide;
(S)-3-(6-((4-(3-aminopiperidin-1-yl)-5-(1-(tetrahydro-2H-pyran-4-yl)-1H-pyrazol-4-yl)pyridin-2-yl)amino)pyridin-2-yl)oxazolidin-2-one;
(S)-N-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)pyrrolidin-3-yl)acetamide;
6-((4-((S)-3-aminopiperidin-1-yl)-5-(1-(tetrahydro-2H-pyran-4-yl)-1H-pyrazol-4-yl)pyridin-2-yl)amino)-2-(2-fluoro-6-methoxyphenyl)nicotinamide;
6-((4-((S)-3-aminopiperidin-1-yl)-5-(1-(tetrahydro-2H-pyran-4-yl)-1H-pyrazol-4-yl)pyridin-2-yl)amino)-2-(2-fluoro-6-methoxyphenyl)-N,N-dimethylnicotinamide;
(S)-N-(4-(3-aminopiperidin-1-yl)-5-(1-methyl-3-(trifluoromethyl)-1H-pyrazol-4-yl)pyridin-2-yl)-2-(2-fluoro-6-methoxyphenyl)pyrimidin-4-amine;
(S)-2-(4-(4-(3-aminopiperidin-1-yl)-6-((2-(2-fluoro-6-methoxyphenyl)pyrimidin-4-yl)amino)pyridin-3-yl)-1H-pyrazol-1-yl)-N,N-dimethylacetamide;
(S)-2-(4-(4-(3-aminopiperidin-1-yl)-6-((2-(2-fluoro-6-methoxyphenyl)pyrimidin-4-yl)amino)pyridin-3-yl)-1H-pyrazol-1-yl)ethan-1-ol;
(S)-N-(4-(3-aminopiperidin-1-yl)-5-(1-(2,2-difluoroethyl)-1H-pyrazol-4-yl)pyridin-2-yl)-2-(2-fluoro-6-methoxyphenyl)pyrimidin-4-amine;
N-(5-(1-(difluoromethyl)-1H-pyrazol-4-yl)-4-(2-methyl-2,8-diazaspiro[4.5]decan-8-yl)pyridin-2-yl)-2-(2-fluoro-6-methoxyphenyl)pyrimidin-4-amine;
2-(2-fluoro-6-methoxyphenyl)-N-(4-(2-methyl-2,8-diazaspiro[4.5]decan-8-yl)-5-(1-(2,2,2-trifluoroethyl)-1H-pyrazol-4-yl)pyridin-2-yl)pyrimidin-4-amine;
N-(2-(2-fluoro-6-methoxyphenyl)pyrimidin-4-yl)-4-(2-methyl-2,8-diazaspiro[4.5]decan-8-yl)-6′-morpholino-[3,3′-bipyridin]-6-amine;
(S)-3-(6-((4-(3-aminopiperidin-1-yl)-6′-morpholino-[3,3′-bipyridin]-6-yl)amino)pyridin-2-yl)oxazolidin-2-one;
2-(2-fluoro-6-methoxyphenyl)-6-((4-((S)-3-hydroxypiperidin-1-yl)-5-((tetrahydro-2H-pyran-4-yl)ethynyl)pyridin-2-yl)amino)nicotinic acid;
2-(2-fluoro-6-methoxyphenyl)-6-((4-((S)-3-hydroxypiperidin-1-yl)-5-((tetrahydro-2H-pyran-4-yl)ethynyl)pyridin-2-yl)amino)nicotinamide;
6-((5-(1-(difluoromethyl)-1H-pyrazol-4-yl)-4-((S)-3-hydroxypiperidin-1-yl)pyridin-2-yl)amino)-2-(2-fluoro-6-methoxyphenyl)-N,N-dimethylnicotinamide;
6-((5-(1-(difluoromethyl)-1H-pyrazol-4-yl)-4-((S)-3-hydroxypiperidin-1-yl)pyridin-2-yl)amino)-2-(2-fluoro-6-methoxyphenyl)nicotinamide;
2-(2-fluoro-6-methoxyphenyl)-6-((4-((S)-3-hydroxypiperidin-1-yl)-5-(1-(2,2,2-tri fluoroethyl)-1H-pyrazol-4-yl)pyridin-2-yl)amino)nicotinamide;
2-(2-fluoro-6-methoxyphenyl)-6-((4-((S)-3-hydroxypiperidin-1-yl)-5-(1-(2-methoxyethyl)-1H-pyrazol-4-yl)pyridin-2-yl)amino)nicotinamide;
(3S)-1-(5-(1-(difluoromethyl)-1H-pyrazol-4-yl)-2-((6-(2-fluoro-6-methoxyphenyl)-5-nitropyridin-2-yl)amino)pyridin-4-yl)piperidin-3-ol;
4-((S)-3-aminopiperidin-1-yl)-5-(1-(difluoromethyl)-1H-pyrazol-4-yl)-N-(6-(2-fluoro-6-methoxyphenyl)-5-nitropyridin-2-yl)pyridin-2-amine;
N-(5-(1-(difluoromethyl)-1H-pyrazol-4-yl)-4-(4-(2-(dimethylamino)ethyl)piperidin-1-yl)pyridin-2-yl)-2-(2-fluoro-6-methoxyphenyl)pyrimidin-4-amine;
N-(4-(4-(2-(dimethylamino)ethyl)piperidin-1-yl)-5-(1-(2,2,2-trifluoroethyl)-1H-pyrazol-4-yl)pyridin-2-yl)-2-(2-fluoro-6-methoxyphenyl)pyrimidin-4-amine;
4-(4-(2-(dimethylamino)ethyl)piperidin-1-yl)-N-(2-(2-fluoro-6-methoxyphenyl)pyrimidin-4-yl)-6′-morpholino-[3,3′-bipyridin]-6-amine;
(3S)-1-(2-((5-amino-6-(2-fluoro-6-methoxyphenyl)pyridin-2-yl)amino)-5-(1-(difluoromethyl)-1H-pyrazol-4-yl)pyridin-4-yl)piperidin-3-ol;
N²-(4-((S)-3-aminopiperidin-1-yl)-5-(1-(difluoromethyl)-1H-pyrazol-4-yl)pyridin-2-yl)-6-(2-fluoro-6-methoxyphenyl)pyridin-2,5-diamine;
4-((S)-3-aminopiperidin-1-yl)-5-(1-(2,2-difluoroethyl)-1H-pyrazol-4-yl)-N-(6-(2-fluoro-6-methoxyphenyl)-5-nitropyridin-2-yl)pyridin-2-amine;
4-((S)-3-aminopiperidin-1-yl)-N-(6-(2-fluoro-6-methoxyphenyl)-5-nitropyridin-2-yl-) 5-(1-(2,2,2-trifluoroethyl)-1H-pyrazol-4-yl)pyridin-2-amine;
4-((S)-3-aminopiperidin-1-yl)-N-(6-(2-fluoro-6-methoxyphenyl)-5-nitropyridin-2-yl-) 5-(1-(trifluoromethyl)-1H-pyrazol-4-yl)pyridin-2-amine;
4-((S)-3-aminopiperidin-1-yl)-N-(6-(2-fluoro-6-methoxyphenyl)-5-nitropyridin-2-yl-) 5-((tetrahydro-2H-pyran-4-yl)ethynyl)pyridin-2-amine;
4-((S)-3-aminopiperidin-1-yl)-N-(6-(2-fluoro-6-methoxyphenyl)-5-nitropyridin-2-yl-) 5-((1-(trifluoromethyl)-1H-pyrazol-4-yl)ethynyl)pyridin-2-amine;
(S)-N-(4-(3-aminopiperidin-1-yl)-5-(1-(trifluoromethyl)-1H-pyrazol-4-yl)pyridin-2-yl)-2-(2,4-difluoro-6-methoxyphenyl)pyrimidin-4-amine;
(S)-N-(4-(3-aminopiperidin-1-yl)-5-(1-(2,2-difluoroethyl)-1H-pyrazol-4-yl)pyridin-2-yl)-2-(2,4-difluoro-6-methoxyphenyl)pyrimidin-4-amine;
(S)-N-(4-(3-aminopiperidin-1-yl)-5-((1-(trifluoromethyl)-1H-pyrazol-4-yl)ethynyl)pyridin-2-yl)-2-(2,4-difluoro-6-methoxyphenyl)pyrimidin-4-amine;
6-((5-(1-(2,2-difluoroethyl)-1H-pyrazol-4-yl)-4-((S)-3-hydroxypiperidin-1-yl)pyridin-2-yl)amino)-2-(2-fluoro-6-methoxyphenyl)nicotinamide;
(S)-1-(2-((2-(2-fluoro-4-(hydroxymethyl)-6-methoxyphenyl)pyrimidin-4-yl)amino)-5-(1-(tetrahydro-2H-pyran-4-yl)-1H-pyrazol-4-yl)pyridin-4-yl)piperidin-3-ol;
(S)-N-(4-(3-aminopiperidin-1-yl)-5-(1-(2-morpholinoethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(2-(pyrrolidin-1-yl)ethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-((1-(difluoromethyl)-1H-pyrazol-4-yl)ethynyl)pyridin-2-yl)-2-(2-fluoro-6-methoxyphenyl)pyrimidin-4-amine;
(S)-N-(4-(3-aminopiperidin-1-yl)-5-((1-(2,2-difluoroethyl)-1H-pyrazol-4-yl)ethynyl)pyridin-2-yl)-2-(2-fluoro-6-methoxyphenyl)pyrimidin-4-amine;
(S)-N-(4-(3-aminopiperidin-1-yl)-5-((1-(2,2,2-trifluoroethyl)-1H-pyrazol-4-yl)ethynyl)pyridin-2-yl)-2-(2-fluoro-6-methoxyphenyl)pyrimidin-4-amine;
(S)-3-(4-(4-(3-aminopiperidin-1-yl)-6-((2-(2-fluoro-6-methoxyphenyl)pyrimidin-4-yl)amino)pyridin-3-yl)-1H-pyrazol-1-yl)propanamide;
(S)-N-(4-(3-aminopiperidin-1-yl)-5-(1-(azetidin-3-yl)-1H-pyrazol-4-yl)pyridin-2-yl)-2-(2-fluoro-6-methoxyphenyl)pyrimidin-4-amine;
N²-(4-((S)-3-aminopiperidin-1-yl)-5-(1-(2,2-difluoroethyl)-1H-pyrazol-4-yl)pyridin-2-yl)-6-(2-fluoro-6-methoxyphenyl)pyridin-2,5-diamine;
N²-(4-((S)-3-aminopiperidin-1-yl)-5-(1-(2,2,2-trifluoroethyl)-1H-pyrazol-4-yl)pyridin-2-yl)-6-(2-fluoro-6-methoxyphenyl)pyridin-2,5-diamine;
(S)-1-(2-((2-(2,4-difluoro-6-methoxyphenyl)pyrimidin-4-yl)amino)-5-(1-(trifluoro
methyl)-1H-pyrazol-4-yl)pyridin-4-yl)piperidin-3-ol;
(S)-1-(2-((2-(2,4-difluoro-6-methoxyphenyl)pyrimidin-4-yl)amino)-5-(1-(2,2-difluoroethyl)-1H-pyrazol-4-yl)pyridin-4-yl)piperidin-3-ol;
N²-(4-((S)-3-aminopiperidin-1-yl)-5-(1-(trifluoromethyl)-1H-pyrazol-4-yl)pyridin-2-yl)-6-(2-fluoro-6-methoxyphenyl)pyridin-2,5-diamine;
(S)-1-(2-((2-(2,4-difluoro-6-methoxyphenyl)pyrimidin-4-yl)amino)-5-(1-(2,2,2-tri fluoroethyl)-1H-pyrazol-4-yl)pyridin-4-yl)piperidin-3-ol;
(S)-5-(3-aminopiperidin-1-yl)-N-(2-(2-fluoro-6-methoxyphenyl)pyrimidin-4-yl)-6-(1-(2,2,2-trifluoroethyl)-1H-pyrazol-4-yl)pyridazin-3-amine;
(S)-(4-(4-(3-aminopiperidin-1-yl)-6-((2-(2-fluoro-6-methoxyphenyl)pyrimidin-4-yl)amino)pyridin-3-yl)phenyl)(4-methylpiperazin-1-yl)methanone;
(3S)-1-(2-((5-amino-6-(2-fluoro-6-methoxyphenyl)pyridin-2-yl)amino)-5-(1-(tetrahydro-2H-pyran-4-yl)-1H-pyrazol-4-yl)pyridin-4-yl)piperidin-3-ol;
(3S)-1-(2-((5-amino-6-(2-fluoro-6-methoxyphenyl)pyridin-2-yl)amino)-5-(1-(trifluoromethyl)-1H-pyrazol-4-yl)pyridin-4-yl)piperidin-3-ol;
(3S)-1-(2-((5-amino-6-(2-fluoro-6-methoxyphenyl)pyridin-2-yl)amino)-5-(1-(2,2-di fluoroethyl)-1H-pyrazol-4-yl)pyridin-4-yl)piperidin-3-ol;
(3S)-1-(2-((5-amino-6-(2-fluoro-6-methoxyphenyl)pyridin-2-yl)amino)-5-(1-(2,2,2-trifluoroethyl)-1H-pyrazol-4-yl)pyridin-4-yl)piperidin-3-ol;
N²-(4-((S)-3-aminopiperidin-1-yl)-5-(1-(tetrahydro-2H-pyran-4-yl)-1H-pyrazol-4-yl)pyridin-2-yl)-6-(2-fluoro-6-methoxyphenyl)pyridin-2,5-diamine;
(S)-1-(2-((2-(2,4-difluoro-6-methoxyphenyl)pyrimidin-4-yl)amino)-5-(1-(difluoromethyl)-1H-pyrazol-4-yl)pyridin-4-yl)piperidin-3-ol;
(3S)-1-(2-((6-(2,4-difluoro-6-methoxyphenyl)-5-nitropyridin-2-yl)amino)-5-(1-(di fluoromethyl)-1H-pyrazol-4-yl)pyridin-4-yl)piperidin-3-ol;
(3S)-1-(2-((5-amino-6-(2,4-difluoro-6-methoxyphenyl)pyridin-2-yl)amino)-5-(1-(di fluoromethyl)-1H-pyrazol-4-yl)pyridin-4-yl)piperidin-3-ol;
methyl (S)-4-(4-((4-(3-aminopiperidin-1-yl)-5-(1-(difluoromethyl)-1H-pyrazol-4-yl)pyridin-2-yl)amino)pyrimidin-2-yl)-3-fluoro-5-methoxybenzoate;
(S)-4-(4-((4-(3-aminopiperidin-1-yl)-5-(1-(difluoromethyl)-1H-pyrazol-4-yl)pyridin-2-yl)amino)pyrimidin-2-yl)-3-fluoro-5-methoxy-N-methylbenzamide;
(S)-4-(4-((4-(3-aminopiperidin-1-yl)-5-(1-(difluoromethyl)-1H-pyrazol-4-yl)pyridin-2-yl)amino)pyrimidin-2-yl)-3-fluoro-5-methoxy-N,N-dimethylbenzamide;
(S)-1-(2-((2-(4-((3-hydroxyazetidin-1-yl)methyl)thiophen-2-yl)pyrimidin-4-yl)amino)-5-(1-(tetrahydro-2H-pyran-4-yl)-1H-pyrazol-4-yl)pyridin-4-yl)piperidin-3-ol;
(S)-3-fluoro-4-(4-((4-(3-hydroxypiperidin-1-yl)-5-(1-(tetrahydro-2H-pyran-4-yl)-1H-pyrazol-4-yl)pyridin-2-yl)amino)pyrimidin-2-yl)-5-methoxybenzaldehyde;
(S)-1-(2-((2-(2-fluoro-6-methoxy-4-((methylamino)methyl)phenyl)pyrimidin-4-yl)amino)-5-(1-(tetrahydro-2H-pyran-4-yl)-1H-pyrazol-4-yl)pyridin-4-yl)piperidin-3-ol;
6-((6-(2-fluoro-6-methoxyphenyl)-5-nitropyridin-2-yl)amino)-4-((S)-3-hydroxypiperidin-1-yl)nicotinamide;
6-((2-(2-fluoro-6-methoxyphenyl)pyrimidin-4-yl)amino)-4-((S)-3-hydroxypiperidin-1-yl)-N-((R)-1-methylpyrrolidin-3-yl)nicotinamide;
6-((5-amino-6-(2-fluoro-6-methoxyphenyl)pyridin-2-yl)amino)-4-((S)-3-hydroxypiperidin-1-yl)nicotinamide;
6-((6-(2-fluoro-6-methoxyphenyl)-5-nitropyridin-2-yl)amino)-N-((1R,3R)-3-hydroxycyclopentyl)-4-((S)-3-hydroxypiperidin-1-yl)nicotinamide;
6-((6-(2-fluoro-6-methoxyphenyl)-5-nitropyridin-2-yl)amino)-N-((1R,3S)-3-hydroxycyclopentyl)-4-((S)-3-hydroxypiperidin-1-yl)nicotinamide;
6-((6-(2-fluoro-6-methoxyphenyl)-5-nitropyridin-2-yl)amino)-N-(3-hydroxycyclobut yl)-4-((S)-3-hydroxypiperidin-1-yl)nicotinamide;
6-((6-(2-fluoro-6-methoxyphenyl)-5-nitropyridin-2-yl)amino)-N-((1R,2R)-2-hydroxycyclobutyl)-4-((S)-3-hydroxypiperidin-1-yl)nicotinamide;
N-(3,3-difluorocyclobutyl)-6-((6-(2-fluoro-6-methoxyphenyl)-5-nitropyridin-2-yl)amino)-4-((S)-3-hydroxypiperidin-1-yl)nicotinamide;
N-((1R,3R)-3-aminocyclopentyl)-6-((6-(2-fluoro-6-methoxyphenyl)-5-nitropyridin-2-yl)amino)-4-((S)-3-hydroxypiperidin-1-yl)nicotinamide;
N-((1R,3S)-3-aminocyclopentyl)-6-((6-(2-fluoro-6-methoxyphenyl)-5-nitropyridin-2-yl)amino)-4-((S)-3-hydroxypiperidin-1-yl)nicotinamide;
6-((5-amino-6-(2-fluoro-6-methoxyphenyl)pyridin-2-yl)amino)-N-((1R,3R)-3-hydroxycyclopentyl)-4-((S)-3-hydroxypiperidin-1-yl)nicotinamide;
6-((5-amino-6-(2-fluoro-6-methoxyphenyl)pyridin-2-yl)amino)-N-((1R,3S)-3-hydroxycyclopentyl)-4-((S)-3-hydroxypiperidin-1-yl)nicotinamide;
6-((6-(2-fluoro-6-methoxyphenyl)-5-nitropyridin-2-yl)amino)-4-((S)-3-hydroxypiperidin-1-yl)-N-((R)-1-methylpyrrolidin-3-yl)nicotinamide;
N-(4-((3S,4R)-3-amino-4-methylpiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3S,5S)-3-amino-5-methylpiperidin-1-yl)-5-(1-(difluoromethyl)-1H-pyrazol-4-yl)pyridin-2-yl)-2-(2-fluoro-6-methoxyphenyl)pyrimidin-4-amine;
(S)-N-(4-(3-amino-3-methylpiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3R,4S)-3-amino-4-fluoropiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3R,4R)-3-amino-4-fluoropiperidin-1-yl)-5-(1-(difluoromethyl)-1H-pyrazol-4-yl)pyridin-2-yl)-2-(2-fluoro-6-methoxyphenyl)pyrimidin-4-amine;
(3R,4R)-3-amino-1-(5-(1-(difluoromethyl)-1H-pyrazol-4-yl)-2-((2-(2-fluoro-6-methoxyphenyl)pyrimidin-4-yl)amino)pyridin-4-yl)piperidin-4-ol;
N-(4-((3S,5S)-3-amino-5-methylpiperidin-1-yl)-5-(1-(difluoromethyl)-1H-pyrazol-4-yl)pyridin-2-yl)-2-(2,4-difluoro-6-methoxyphenyl)pyrimidin-4-amine;
N-(4-((3S,4R)-3-amino-4-methylpiperidin-1-yl)-5-(1-(difluoromethyl)-1H-pyrazol-4-yl)pyridin-2-yl)-2-(2,4-difluoro-6-methoxyphenyl)pyrimidin-4-amine;
(S)-N-(4-(3-amino-3-methylpiperidin-1-yl)-5-(1-(difluoromethyl)-1H-pyrazol-4-yl)pyridin-2-yl)-2-(2,4-difluoro-6-methoxyphenyl)pyrimidin-4-amine;
(3R,4R)-3-amino-1-(2-((2-(2,4-difluoro-6-methoxyphenyl)pyrimidin-4-yl)amino)-5-(1-(difluoromethyl)-1H-pyrazol-4-yl)pyridin-4-yl)piperidin-4-ol;
(1S,4S)-4-((5-(1-(difluoromethyl)-1H-pyrazol-4-yl)-2-((2-(2-fluoro-6-methoxyphenyl)pyrimidin-4-yl)amino)pyridin-4-yl)(methyl)amino)cyclohexan-1-ol;
N²-(4-((3S,4R)-3-amino-4-methylpiperidin-1-yl)-5-(1-(difluoromethyl)-1H-pyrazol-4-yl)pyridin-2-yl)-6-(2,4-difluoro-6-methoxyphenyl)pyridin-2,5-diamine;
N²-(4-((3R,4R)-3-amino-4-fluoropiperidin-1-yl)-5-(1-(difluoromethyl)-1H-pyrazol-4-yl)pyridin-2-yl)-6-(2,4-difluoro-6-methoxyphenyl)pyridin-2,5-diamine;
(S)-1-(2-((2-(2,4-difluoro-6-methoxyphenyl)pyrimidin-4-yl)amino)-5-((1-(difluoromethyl)-1H-pyrazol-4-yl)ethynyl)pyridin-4-yl)piperidin-3-ol;
(S)-1-(2-((2-(2,4-difluoro-6-methoxyphenyl)pyrimidin-4-yl)amino)-5-((1-(trifluoromethyl)-1H-pyrazol-4-yl)ethynyl)pyridin-4-yl)piperidin-3-ol;
(S)-1-(2-((2-(2,4-difluoro-6-methoxyphenyl)pyrimidin-4-yl)amino)-5-((1-(2,2-difluoroethyl)-1H-pyrazol-4-yl)ethynyl)pyridin-4-yl)piperidin-3-ol;
(S)-1-(2-((2-(2,4-difluoro-6-methoxyphenyl)pyrimidin-4-yl)amino)-5-((1-(2,2,2-trifluoroethyl)-1H-pyrazol-4-yl)ethynyl)pyridin-4-yl)piperidin-3-ol;
(3S)-1-(5-((1-(2,2-difluoroethyl)-1H-pyrazol-4-yl)ethynyl)-2-((6-(2-fluoro-6-methoxyphenyl)-5-nitropyridin-2-yl)amino)pyridin-4-yl)piperidin-3-ol;
(3S)-1-(5-((1-(difluoromethyl)-1H-pyrazol-4-yl)ethynyl)-2-((6-(2-fluoro-6-methoxyphenyl)-5-nitropyridin-2-yl)amino)pyridin-4-yl)piperidin-3-ol;
(3S)-1-(2-((6-(2,4-difluoro-6-methoxyphenyl)-5-nitropyridin-2-yl)amino)-5-(1-(2,2-difluoroethyl)-1H-pyrazol-4-yl)pyridin-4-yl)piperidin-3-ol;
(3S)-1-(2-((6-(2,4-difluoro-6-methoxyphenyl)-5-nitropyridin-2-yl)amino)-5-(1-(tetrahydro-2H-pyran-4-yl)-1H-pyrazol-4-yl)pyridin-4-yl)piperidin-3-ol;
(3S)-1-(2-((6-(2,4-difluoro-6-methoxyphenyl)-5-nitropyridin-2-yl)amino)-5-((tetrahydro-2H-pyran-4-yl)ethynyl)pyridin-4-yl)piperidin-3-ol;
(3S)-1-(2-((5-amino-6-(2,4-difluoro-6-methoxyphenyl)pyridin-2-yl)amino)-5-(1-(2, 2-difluoroethyl)-1H-pyrazol-4-yl)pyridin-4-yl)piperidin-3-ol;
(3S)-1-(2-((5-amino-6-(2,4-difluoro-6-methoxyphenyl)pyridin-2-yl)amino)-5-(1-(tetrahydro-2H-pyran-4-yl)-1H-pyrazol-4-yl)pyridin-4-yl)piperidin-3-ol;
(3S)-1-(2-((5-amino-6-(2,4-difluoro-6-methoxyphenyl)pyridin-2-yl)amino)-5-(2-(tetrahydro-2H-pyran-4-yl)ethyl)pyridin-4-yl)piperidin-3-ol;
(S)-N-(4-(3-aminopiperidin-1-yl)-5-(1-(2-methoxy-2-methylpropyl)-1H-pyrazol-4-yl)pyridin-2-yl)-2-(2-fluoro-6-methoxyphenyl)pyrimidin-4-amine;
(S)-4-(3-aminopiperidin-1-yl)-N-(2-(2-fluoro-6-methoxyphenyl)pyrimidin-4-yl)-6′-(2-methoxyethoxy)-[3,3′-bipyridin]-6-amine;
(S)-(3,3-difluoroazetidin-1-yl)(4-(4-((5-(1-(difluoromethyl)-1H-pyrazol-4-yl)-4-(3-hydroxypiperidin-1-yl)pyridin-2-yl)amino)pyrimidin-2-yl)-3-fluoro-5-methoxyphenyl)methanone;
(4-(4-((5-(1-(difluoromethyl)-1H-pyrazol-4-yl)-4-((S)-3-hydroxypiperidin-1-yl)pyridin-2-yl)amino)pyrimidin-2-yl)-3-fluoro-5-methoxyphenyl)((S)-3-fluoropyrrolidin-1-yl)methanone;
(4-(4-((5-(1-(difluoromethyl)-1H-pyrazol-4-yl)-4-((S)-3-hydroxypiperidin-1-yl)pyridin-2-yl)amino)pyrimidin-2-yl)-3-fluoro-5-methoxyphenyl)((R)-3-fluoropyrrolidin-1-yl)methanone;
(S)-(4-(4-((5-(1-(difluoromethyl)-1H-pyrazol-4-yl)-4-(3-hydroxypiperidin-1-yl)pyridin-2-yl)amino)pyrimidin-2-yl)-3-fluoro-5-methoxyphenyl)(3,3-difluoropyrrolidin-1-yl)methanone;
(S)-1-(4-(4-(3-aminopiperidin-1-yl)-6-((2-(2-fluoro-6-methoxyphenyl)pyrimidin-4-yl)amino)pyridin-3-yl)-1H-pyrazol-1-yl)-2-methylpropan-2-ol;
(S)-N-(4-(3-aminopiperidin-1-yl)-5-(1-(3-(dimethylamino)propyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(azetidin-3-ylmethyl)-1H-pyrazol-4-yl)pyridin-2-yl)-2-(2-fluoro-6-methoxyphenyl)pyrimidin-4-amine;
N-(4-((S)-3-aminopiperidin-1-yl)-5-(1-((R)-pyrrolidin-3-yl)-1H-pyrazol-4-yl)pyridin-2-yl)-2-(2-fluoro-6-methoxyphenyl)pyrimidin-4-amine;
N-(4-((S)-3-aminopiperidin-1-yl)-5-(1-((S)-pyrrolidin-3-yl)-1H-pyrazol-4-yl)pyridin-2-yl)-2-(2-fluoro-6-methoxyphenyl)pyrimidin-4-amine;
(S)-4-(3-aminopiperidin-1-yl)-N⁶-(2-(dimethylamino)ethyl)-N⁶-(2-(2-fluoro-6-methoxyphenyl)pyrimidin-4-yl)-[3,3′-bipyridin]-6,6′-diamine;
(S)-4-(3-aminopiperidin-1-yl)-N-(2-(2-fluoro-6-methoxyphenyl)pyrimidin-4-yl)-6′-(piperazin-1-yl)-[3,3′-bipyridin]-6-amine;
(S)-(2-(4-((4-(3-aminopiperidin-1-yl)-5-(1-(difluoromethyl)-1H-pyrazol-4-yl)pyridin-2-yl)amino)pyrimidin-2-yl)-3-fluorophenyl)methanol;
(S)-(2-(4-((4-(3-aminopiperidin-1-yl)-5-((1-(difluoromethyl)-1H-pyrazol-4-yl)ethynyl)pyridin-2-yl)amino)pyrimidin-2-yl)-3-fluorophenyl)methanol;
(S)-N-(4-(3-aminopiperidin-1-yl)-5-(1-(1-methoxy-2-methylpropan-2-yl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-((3,3-difluorocyclobutyl)methyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(2-(dimethylamino)ethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(fluoromethyl)-1H-pyrazol-4-yl)pyridin-2-yl)-2-(2-fluoro-6-methoxyphenyl)pyrimidin-4-amine;
(S)-4-(3-aminopiperidin-1-yl)-N-(2-(2-fluoro-6-methoxyphenyl)pyrimidin-4-yl)-6′-(piperidin-4-yl)-[3,3′-bipyridin]-6-amine;
(S)-4-(3-aminopiperidin-1-yl)-6′-(2-(dimethylamino)ethoxy)-N-(2-(2-fluoro-6-methoxyphenyl)pyrimidin-4-yl)-[3,3′-bipyridin]-6-amine;
(S)-1-(4′-(3-aminopiperidin-1-yl)-6′-((2-(2-fluoro-6-methoxyphenyl)pyrimidin-4-yl)amino)-[3,3′-bipyridin]-6-yl)-3-ethylurea;
(4-(4-((4-((S)-3-aminopiperidin-1-yl)-5-(1-(difluoromethyl)-1H-pyrazol-4-yl)pyridin-2-yl)amino)pyrimidin-2-yl)-3-fluoro-5-methoxyphenyl)((R)-3-fluoropyrrolidin-1-yl)methanone;
N-(4-((2R,5S)-5-amino-2-methylpiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3R,4S)-3-amino-4-methoxypiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3R,4R)-3-amino-4-methoxypiperidin-1-yl)-5-(1-(difluoromethyl)-1H-pyrazol-4-yl)pyridin-2-yl)-2-(2-fluoro-6-methoxyphenyl)pyrimidin-4-amine;
(S)-(4-(4-(3-aminopiperidin-1-yl)-6-((2-(2-fluoro-6-methoxyphenyl)pyrimidin-4-yl)amino)pyridin-3-yl)phenyl)(morpholino)methanone;
(S)-N-(4-(3-aminopiperidin-1-yl)-5-(4-methyl-3,4-dihydro-2H-pyrido[3,2-b][1,4]oxazin-7-yl)pyridin-2-yl)-2-(2-fluoro-6-methoxyphenyl)pyrimidin-4-amine;
(S)-N-(4-(3-aminopiperidin-1-yl)-5-(4-methyl-3,4-dihydro-2H-benzo[b][1,4]oxazin-7-yl)pyridin-2-yl)-2-(2-fluoro-6-methoxyphenyl)pyrimidin-4-amine;
(S)-6-(4-(3-aminopiperidin-1-yl)-6-((2-(2-fluoro-6-methoxyphenyl)pyrimidin-4-yl)amino)pyridin-3-yl)-1,4-dihydro-2H-benzo[d][1,3]oxazin-2-one;
(S)-1-(4-(4-(3-aminopiperidin-1-yl)-6-((2-(2-fluoro-6-methoxyphenyl)pyrimidin-4-yl)amino)pyridin-3-yl)phenyl)pyrrolidin-2-one;
(S)-7-(4-(3-aminopiperidin-1-yl)-6-((2-(2-fluoro-6-methoxyphenyl)pyrimidin-4-yl)amino)pyridin-3-yl)-2H-benzo[b][1,4]oxazin-3(4H)-one;
(S)-7-(4-(3-aminopiperidin-1-yl)-6-((2-(2-fluoro-6-methoxyphenyl)pyrimidin-4-yl)amino)pyridin-3-yl)-4-methyl-2H-benzo[b][1,4]oxazin-3(4H)-one;
(S)-6-(4-(3-aminopiperidin-1-yl)-6-((2-(2-fluoro-6-methoxyphenyl)pyrimidin-4-yl)amino)pyridin-3-yl)-8-methyl-3,4-dihydroquinolin-2(1H)-one;
(S)-6-(4-(3-aminopiperidin-1-yl)-6-((2-(2-fluoro-6-methoxyphenyl)pyrimidin-4-yl)amino)pyridin-3-yl)-1-methyl-3,4-dihydroquinolin-2(1H)-one;
(S)-1-(2-((2-(2-fluoro-6-methoxyphenyl)pyrimidin-4-yl)amino)-5-(1-(2,2,6,6-tetramethyltetrahydro-2H-pyran-4-yl)-1H-pyrazol-4-yl)pyridin-4-yl)piperidin-3-ol;
(S)-(4-(4-(3-aminopiperidin-1-yl)-6-((2-(2-fluoro-6-methoxyphenyl)pyrimidin-4-yl)amino)pyridin-3-yl)phenyl)(piperazin-1-yl)methanone;
(S)-N-(5-(1-(difluoromethyl)-1H-pyrazol-4-yl)-4-(3-((methylamino)methyl)piperidin-1-yl)pyridin-2-yl)-2-(2-fluoro-6-methoxyphenyl)pyrimidin-4-amine;
(R)-N-(5-(1-(difluoromethyl)-1H-pyrazol-4-yl)-4-(3-((methylamino)methyl)piperidin-1-yl)pyridin-2-yl)-2-(2-fluoro-6-methoxyphenyl)pyrimidin-4-amine;
N-(4-((3R,4R)-4-amino-3-methylpiperidin-1-yl)-5-(1-(difluoromethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(2,2,6,6-tetramethyltetrahydro-2H-pyran-4-yl)-1H-pyrazol-4-yl)pyridin-2-yl)-2-(2-fluoro-6-methoxyphenyl)pyrimidin-4-amine;
6-(4-((S)-3-aminopiperidin-1-yl)-6-((2-(2-fluoro-6-methoxyphenyl)pyrimidin-4-yl)amino)pyridin-3-yl)-2-methyl-2H-benzo[b][1,4]oxazin-3(4H)-one;
(S)-N-(5-(1-(difluoromethyl)-1H-pyrazol-4-yl)-4-(3-methylpiperidin-1-yl)pyridin-2-yl)-2-(2-fluoro-6-methoxyphenyl)pyrimidin-4-amine;
(S)-N-(5-(1-(difluoromethyl)-1H-pyrazol-4-yl)-4-(3-(fluoromethyl)piperidin-1-yl)pyridin-2-yl)-2-(2-fluoro-6-methoxyphenyl)pyrimidin-4-amine;
(R)-N-(5-(1-(difluoromethyl)-1H-pyrazol-4-yl)-4-(3-(difluoromethyl)piperidin-1-yl)pyridin-2-yl)-2-(2-fluoro-6-methoxyphenyl)pyrimidin-4-amine;
(S)-7-(4-(3-aminopiperidin-1-yl)-6-((2-(2-fluoro-6-methoxyphenyl)pyrimidin-4-yl)amino)pyridin-3-yl)-4-methyl-2H-pyrido[3,2-b][1,4]oxazin-3(4H)-one;
(S)-7-(4-(3-aminopiperidin-1-yl)-6-((2-(2-fluoro-6-methoxyphenyl)pyrimidin-4-yl)amino)pyridin-3-yl)-2H-pyrido[3,2-b][1,4]oxazin-3(4H)-one;
(S)-6-(4-(3-aminopiperidin-1-yl)-6-((2-(2-fluoro-6-methoxyphenyl)pyrimidin-4-yl)amino)pyridin-3-yl)-3,4-dihydro-1,8-naphthyridin-2(1H)-one;
(S)-N-(4-(3-aminopiperidin-1-yl)-5-(1-(oxetan-3-yl)-1H-pyrazol-4-yl)pyridin-2-yl)-2-(2-fluoro-6-methoxyphenyl)pyrimidin-4-amine;
methyl (S)-2-(4-(4-(3-aminopiperidin-1-yl)-6-((2-(2-fluoro-6-methoxyphenyl)pyrimidin-4-yl)amino)pyridin-3-yl)-1H-pyrazol-1-yl)-2-methylpropanoate;
(S)-N-(4-(4-(3-aminopiperidin-1-yl)-6-((2-(2-fluoro-6-methoxyphenyl)pyrimidin-4-yl)amino)pyridin-3-yl)phenyl)acetamide;
(S)-3-(4-(3-aminopiperidin-1-yl)-6-((2-(2-fluoro-6-methoxyphenyl)pyrimidin-4-yl)amino)pyridin-3-yl)-N,N-dimethylbenzamide;
(S)-N-(4-(3-aminopiperidin-1-yl)-5-(3-methyl-4-(trifluoromethoxy)phenyl)pyridin-2-yl)-2-(2-fluoro-6-methoxyphenyl)pyrimidin-4-amine;
(S)-N-(4-(3-aminopiperidin-1-yl)-5-(4-fluoro-3-methylphenyl)pyridin-2-yl)-2-(2-fluoro-6-methoxyphenyl)pyrimidin-4-amine;
(S)-N-(4-(3-aminopiperidin-1-yl)-5-(1-(piperidin-4-yl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(4-isopropoxy-3,5-dimethylphenyl)pyridin-2-yl)-2-(2-fluoro-6-methoxyphenyl)pyrimidin-4-amine;
(S)-N-(4-(3-aminopiperidin-1-yl)-5-(4-methoxy-3,5-dimethylphenyl)pyridin-2-yl)-2-(2-fluoro-6-methoxyphenyl)pyrimidin-4-amine;
(S)-N-(4-(3-aminopiperidin-1-yl)-5-(4-morpholinophenyl)pyridin-2-yl)-2-(2-fluoro-6-methoxyphenyl)pyrimidin-4-amine;
(S)-N-(4-(3-aminopiperidin-1-yl)-5-(3-methyl-4-morpholinophenyl)pyridin-2-yl)-2-(2-fluoro-6-methoxyphenyl)pyrimidin-4-amine;
(S)-(4-(4-(3-aminopiperidin-1-yl)-6-((2-(2-fluoro-6-methoxyphenyl)pyrimidin-4-yl)amino)pyridin-3-yl)phenyl)(4-(2-hydroxyethyl)piperazin-1-yl)methanone;
4-(4-((S)-3-aminopiperidin-1-yl)-6-((2-(2-fluoro-6-methoxyphenyl)pyrimidin-4-yl)amino)pyridin-3-yl)-N-((1r,4S)-4-hydroxycyclohexyl)benzamide;
(S)-4-(4-(3-aminopiperidin-1-yl)-6-((2-(2-fluoro-6-methoxyphenyl)pyrimidin-4-yl)amino)pyridin-3-yl)-2-fluoro-N,N-dimethylbenzamide;
(S)-1-(3-(4-(3-aminopiperidin-1-yl)-6-((2-(2-fluoro-6-methoxyphenyl)pyrimidin-4-yl)amino)pyridin-3-yl)phenyl)ethan-1-one;
(S)-N-(4-(3-aminopiperidin-1-yl)-5-(3-chloro-4-fluorophenyl)pyridin-2-yl)-2-(2-fluoro-6-methoxyphenyl)pyrimidin-4-amine;
(S)-N-(4-(3-aminopiperidin-1-yl)-5-(4-fluoro-3-(trifluoromethyl)phenyl)pyridin-2-yl)-2-(2-fluoro-6-methoxyphenyl)pyrimidin-4-amine;
(S)-N-(4-(3-aminopiperidin-1-yl)-5-(3,4-difluorophenyl)pyridin-2-yl)-2-(2-fluoro-6-methoxyphenyl)pyrimidin-4-amine;
(3R,4R)-4-amino-1-(5-(1-(difluoromethyl)-1H-pyrazol-4-yl)-2-((2-(2-fluoro-6-methoxyphenyl)pyrimidin-4-yl)amino)pyridin-4-yl)piperidin-3-ol;
(S)-N-(4-(3-aminopiperidin-1-yl)-5-(3-(dimethylamino)-4-fluorophenyl)pyridin-2-yl)-2-(2-fluoro-6-methoxyphenyl)pyrimidin-4-amine;
N-(4-((3R,4S)-4-amino-3-methylpiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((S)-3-aminopiperidin-1-yl)-5-(1-((S)-tetrahydrofuran-3-yl)-1H-pyrazol-4-yl)pyridin-2-yl)-2-(2-fluoro-6-methoxyphenyl)pyrimidin-4-amine;
N-(4-((S)-3-aminopiperidin-1-yl)-5-(1-((R)-tetrahydrofuran-3-yl)-1H-pyrazol-4-yl)pyridin-2-yl)-2-(2-fluoro-6-methoxyphenyl)pyrimidin-4-amine;
6-((4-((S)-3-aminopiperidin-1-yl)-5-(1-(tetrahydro-2H-pyran-4-yl)-1H-pyrazol-4-yl)pyridin-2-yl)amino)-2-(2-fluoro-6-methoxyphenyl)nicotinonitrile;
6-((4-((S)-3-aminopiperidin-1-yl)-5-(1-(difluoromethyl)-1H-pyrazol-4-yl)pyridin-2-yl)amino)-2-(2-fluoro-6-methoxyphenyl)nicotinonitrile;
N-(4-((3S,4S)-4-amino-3-fluoropiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3S,5R)-3-amino-5-fluoropiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3S,5S)-3-amino-5-fluoropiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3S,5R)-3-amino-5-(trifluoromethyl)piperidin-1-yl)-5-(1-(difluoromethyl)-1H-pyrazol-4-yl)pyridin-2-yl)-2-(2-fluoro-6-methoxyphenyl)pyrimidin-4-amine;
(3S,5S)-5-amino-1-(5-(1-(difluoromethyl)-1H-pyrazol-4-yl)-2-((2-(2-fluoro-6-methoxyphenyl)pyrimidin-4-yl)amino)pyridin-4-yl)piperidin-3-ol;
N-(4-((3R,4S)-4-amino-3-fluoropiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3S,4R)-4-amino-3-fluoropiperidin-1-yl)-5-(1-(difluoromethyl)-1H-pyrazol-4-yl)pyridin-2-yl)-2-(2-fluoro-6-methoxyphenyl)pyrimidin-4-amine;
6-((4-((S)-3-aminopiperidin-1-yl)-5-(1-(tetrahydro-2H-pyran-4-yl)-1H-pyrazol-4-yl)pyridin-2-yl)amino)-2-(2,4-difluoro-6-methoxyphenyl)nicotinonitrile;
6-((4-((S)-3-aminopiperidin-1-yl)-5-(1-(difluoromethyl)-1H-pyrazol-4-yl)pyridin-2-yl)amino)-2-(2,4-difluoro-6-methoxyphenyl)nicotinonitrile;
(S)-N-(4-(3-aminopiperidin-1-yl)-5-((4-methyltetrahydro-2H-pyran-4-yl)ethynyl)pyridin-2-yl)-2-(2,4-difluoro-6-methoxyphenyl)pyrimidin-4-amine;
(S)-4-((4-(3-aminopiperidin-1-yl)-6-((2-(2,4-difluoro-6-methoxyphenyl)pyrimidin-4-yl)amino)pyridin-3-yl)ethynyl)tetrahydro-2H-pyran-4-ol;
(S)-4-(4-(3-aminopiperidin-1-yl)-6-((2-(2,4-difluoro-6-methoxyphenyl)pyrimidin-4-yl)amino)pyridin-3-yl)-2-methylbut-3-yn-2-ol;
(S)-4-(4-(3-aminopiperidin-1-yl)-6-((2-(2,4-difluoro-6-methoxyphenyl)pyrimidin-4-yl)amino)pyridin-3-yl)but-3-yn-1-ol;
4-((S)-3-aminopiperidin-1-yl)-5-(1-(difluoromethyl)-1H-pyrazol-4-yl)-N-(6-(2-fluoro-6-methoxyphenyl)-5-(methylsulfonyl)pyridin-2-yl)pyridin-2-amine;
2-(2-fluoro-6-methoxyphenyl)-6-((4-((S)-3-hydroxypiperidin-1-yl)-5-(1-(2,2,2-tri fluoroethyl)-1H-pyrazol-4-yl)pyridin-2-yl)amino)nicotinonitrile;
2-(2,4-difluoro-6-methoxyphenyl)-6-((4-((S)-3-hydroxypiperidin-1-yl)-5-(1-(2,2,2-trifluoroethyl)-1H-pyrazol-4-yl)pyridin-2-yl)amino)nicotinonitrile;
6-((4-((S)-3-aminopiperidin-1-yl)-5-(1-(2,2,2-trifluoroethyl)-1H-pyrazol-4-yl)pyridin-2-yl)amino)-2-(2-fluoro-6-methoxyphenyl)nicotinonitrile;
6-((4-((S)-3-aminopiperidin-1-yl)-5-(1-(2-hydroxy-2-methylpropyl)-1H-pyrazol-4-yl)pyridin-2-yl)amino)-2-(2-fluoro-6-methoxyphenyl)nicotinonitrile;
6-((4-((S)-3-aminopiperidin-1-yl)-5-(1-(2,2,2-trifluoroethyl)-1H-pyrazol-4-yl)pyridin-2-yl)amino)-2-(2,4-difluoro-6-methoxyphenyl)nicotinonitrile;
6-((4-((3R,4R)-3-amino-4-fluoropiperidin-1-yl)-5-(1-(difluoromethyl)-1H-pyrazol-4-yl)pyridin-2-yl)amino)-2-(2-fluoro-6-methoxyphenyl)nicotinonitrile;
6-((4-((3R,4S)-3-amino-4-fluoropiperidin-1-yl)-5-(1-(difluoromethyl)-1H-pyrazol-4-yl)pyridin-2-yl)amino)-2-(2-fluoro-6-methoxyphenyl)nicotinonitrile;
6-((4-((S)-3-aminopiperidin-1-yl)-6′-morpholino-[3,3′-bipyridin]-6-yl)amino)-2-(2-fluoro-6-methoxyphenyl)nicotinonitrile;
N-(4-((3S,5R)-3-amino-5-fluoropiperidin-1-yl)-5-(2-(2,2,2-trifluoroethyl)-1H-pyrazol-4-yl)pyridin-2-yl)-2-(2,4-difluoro-6-methoxyphenyl)pyrimidin-4-amine;
2-(2-fluoro-6-methoxyphenyl)-6-((4-((S)-3-hydroxypiperidin-1-yl)-5-(1-(1-methylpiperidin-4-yl)-1H-pyrazol-4-yl)pyridin-2-yl)amino)nicotinonitrile;
6-((4-((3R,4R)-4-amino-3-hydroxypiperidin-1-yl)-5-(1-(difluoromethyl)-1H-pyrazol-4-yl)pyridin-2-yl)amino)-2-(2-fluoro-6-methoxyphenyl)nicotinonitrile;
6-((4-((S)-3-aminopiperidin-1-yl)-5-(1-ethyl-1H-pyrazol-4-yl)pyridin-2-yl)amino)-2-(2-fluoro-6-methoxyphenyl)nicotinonitrile;
6-((4-((S)-3-aminopiperidin-1-yl)-5-(1-isopropyl-1H-pyrazol-4-yl)pyridin-2-yl)amino)-2-(2-fluoro-6-methoxyphenyl)nicotinonitrile;
6-((4-((S)-3-aminopiperidin-1-yl)-5-(1-cyclopropyl-1H-pyrazol-4-yl)pyridin-2-yl)amino)-2-(2-fluoro-6-methoxyphenyl)nicotinonitrile;
6-((4-((S)-3-aminopiperidin-1-yl)-5-(1-(cyclopropylmethyl)-1H-pyrazol-4-yl)pyridin-2-yl)amino)-2-(2-fluoro-6-methoxyphenyl)nicotinonitrile;
6-((5-(1-(difluoromethyl)-1H-pyrazol-4-yl)-4-((3R,4R)-4-fluoro-3-hydroxypiperidin-1-yl)pyridin-2-yl)amino)-2-(2-fluoro-6-methoxyphenyl)nicotinonitrile;
N-(4-((3S,5S)-3-amino-5-fluoropiperidin-1-yl)-5-(1-(2,2,2-trifluoroethyl)-1H-pyrazol-4-yl)pyridin-2-yl)-2-(2-fluoro-6-methoxyphenyl)pyrimidin-4-amine;
N-(4-((3S,5S)-3-amino-5-fluoropiperidin-1-yl)-5-(1-(2,2,2-trifluoroethyl)-1H-pyrazol-4-yl)pyridin-2-yl)-2-(2,4-difluoro-6-methoxyphenyl)pyrimidin-4-amine;
4-((3R,4R)-3-amino-4-fluoropiperidin-1-yl)-5-(1-(difluoromethyl)-1H-pyrazol-4-yl)-N-(6-(2-fluoro-6-methoxyphenyl)-5-nitropyridin-2-yl)pyridin-2-amine;
N-(4-((3S,5R)-3-amino-5-fluoropiperidin-1-yl)-5-(1-isopropyl-1H-pyrazol-4-yl)pyridin-2-yl)-2-(2,4-difluoro-6-methoxyphenyl)pyrimidin-4-amine;
N-(4-((3S,5S)-3-amino-5-fluoropiperidin-1-yl)-5-(1-isopropyl-1H-pyrazol-4-yl)pyridin-2-yl)-2-(2,4-difluoro-6-methoxyphenyl)pyrimidin-4-amine;
6-((4-((3S,4R)-3-amino-4-methylpiperidin-1-yl)-5-(1-(difluoromethyl)-1H-pyrazol-4-yl)pyridin-2-yl)amino)-2-(2-fluoro-6-methoxyphenyl)-nicotinonitrile;
6-((4-((3S,4R)-3-amino-4-methylpiperidin-1-yl)-5-(1-(difluoromethyl)-1H-pyrazol-4-yl)pyridin-2-yl)amino)-2-(2,4-difluoro-6-methoxyphenyl)nicotinonitrile;
N-(4-((3R,4R)-3-amino-4-fluoropiperidin-1-yl)-5-(1-isopropyl-1H-pyrazol-4-yl)pyridin-2-yl)-2-(2,4-difluoro-6-methoxyphenyl)pyrimidin-4-amine;
6-((4-((3R,4R)-3-amino-4-fluoropiperidin-1-yl)-5-(1-isopropyl-1H-pyrazol-4-yl)pyridin-2-yl)amino)-2-(2,4-difluoro-6-methoxyphenyl)nicotinonitrile; and
4-((3R,4R)-3-amino-4-fluoropiperidin-1-yl)-5-(1-(difluoromethyl)-1H-pyrazol-4-yl)-N-(5-fluoro-6-(2-fluoro-6-methoxyphenyl)pyridin-2-yl)pyridin-2-amine.

In the compound of Formula 1 or pharmaceutically acceptable salt, more preferable compounds include the following compound or pharmaceutically acceptable salt thereof selected from the group consisting of:
(S)-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-ol;
(S)-1-(2-((2-(2-fluoro-6-methoxyphenyl)pyrimidin-4-yl)amino)-5-((tetrahydro-2H-pyran-4-yl)ethynyl)pyridin-4-yl)piperidin-3-ol;
(S)-1-(2-((2-(2-fluoro-6-methoxyphenyl)pyrimidin-4-yl)amino)-5-(1-methyl-1H-pyrazol-4-yl)pyridin-4-yl)piperidin-3-ol;
(S)-1-(2-((2-(2-fluoro-6-methoxyphenyl)pyrimidin-4-yl)amino)-5-(1-isopropyl-1H-pyrazol-4-yl)pyridin-4-yl)piperidin-3-ol;
(3S)-1-(2-((2-(2-fluoro-6-methoxyphenyl)pyrimidin-4-yl)amino)-5-(1-(tetrahydrofuran-3-yl)-1H-pyrazol-4-yl)pyridin-4-yl)piperidin-3-ol;
(S)-1-(2-((2-(2-fluoro-6-methoxyphenyl)pyrimidin-4-yl)amino)-5-(1-(1-methylpiperidin-4-yl)-1H-pyrazol-4-yl)pyridin-4-yl)piperidin-3-ol;
(S)-1-(2-((2-(2-fluoro-6-methoxyphenyl)pyrimidin-4-yl)amino)-5-(1-(2,2,2-trifluoroethyl)-1H-pyrazol-4-yl)pyridin-4-yl)piperidin-3-ol;
(S)-1-(5-(1-(cyclopropylmethyl)-1H-pyrazol-4-yl)-2-((2-(2-fluoro-6-methoxyphenyl)pyrimidin-4-yl)amino)pyridin-4-yl)piperidin-3-ol;
(S)-1-(2-((2-(2-fluoro-6-methoxyphenyl)pyrimidin-4-yl)amino)-5-(4-(tetrahydro-2H-pyran-4-yl)phenyl)pyridin-4-yl)piperidin-3-ol;
(S)-1-(2-((2-(2-fluoro-6-methoxyphenyl)pyrimidin-4-yl)amino)-5-(4-(4-methylpiperazin-1-yl)phenyl)pyridin-4-yl)piperidin-3-ol;
(S)-1-(6-((2-(2-fluoro-6-methoxyphenyl)pyrimidin-4-yl)amino)-6′-morpholino-[3,3′-bipyridin]-4-yl)piperidin-3-ol;
(S)-1-(2-((2-(2,6-difluorophenyl)pyrimidin-4-yl)amino)-5-(1-(tetrahydro-2H-pyran-4-yl)-1H-pyrazol-4-yl)pyridin-4-yl)piperidin-3-ol;
(S)-1-(2-((2-(2,3-difluoro-6-methoxyphenyl)pyrimidin-4-yl)amino)-5-(1-(tetrahydro-2H-pyran-4-yl)-1H-pyrazol-4-yl)pyridin-4-yl)piperidin-3-ol;
(S)-1-(6-((2-(2-fluoro-6-methoxyphenyl)pyrimidin-4-yl)amino)-6′-(tetrahydro-2H-pyran-4-yl)-[3,3′-bipyridin]-4-yl)piperidin-3-ol;
(S)-(6′-((2-(2-fluoro-6-methoxyphenyl)pyrimidin-4-yl)amino)-4′-(3-hydroxypiperidin-1-yl)-[3,3′-bipyridin]-6-yl)(4-hydroxypiperidin-1-yl)methanone;
(S)-1-(6-((2-(2-fluoro-6-methoxyphenyl)pyrimidin-4-yl)amino)-6′-(((S)-3-fluoropyrrolidin-1-yl)methyl)-[3,3′-bipyridin]-4-yl)piperidin-3-ol;
(3S)-1-(2-((5-fluoro-6-(2-fluoro-6-methoxyphenyl)pyridin-2-yl)amino)-5-(1-(tetrahydro-2H-pyran-4-yl)-1H-pyrazol-4-yl)pyridin-4-yl)piperidin-3-ol;
(3S)-1-(2-((6-(2-fluoro-6-methoxyphenyl)-5-nitropyridin-2-yl)amino)-5-(1-(tetrahydro-2H-pyran-4-yl)-1H-pyrazol-4-yl)pyridin-4-yl)piperidin-3-ol;
N-(4-(4-((dimethylamino)methyl)-4-fluoropiperidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
(4-(2,2-difluoroethyl)-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-4-yl)methanol;
(S)-N-(4-(3-aminopiperidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
2-(2-fluoro-6-methoxyphenyl)-6-((4-((S)-3-hydroxypiperidin-1-yl)-5-(1-(tetrahydro-2H-pyran-4-yl)-1H-pyrazol-4-yl)pyridin-2-yl)amino)nicotinonitrile;
methyl 2-(2-fluoro-6-methoxyphenyl)-6-((4-((S)-3-hydroxypiperidin-1-yl)-5-(1-(tetrahydro-2H-pyran-4-yl)-1H-pyrazol-4-yl)pyridin-2-yl)amino)nicotinate;
N-(4-(4-(2-(dimethylamino)ethyl)piperidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
1-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-4-yl)cyclopropan-1-ol;
1-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-4-yl)ethan-1-ol;
2-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-4-yl)-2-methylpropan-1-ol;
(S)-1-(2-((2-(7-fluoroquinoxalin-6-yl)pyrimidin-4-yl)amino)-5-(1-(tetrahydro-2H-pyran-4-yl)-1H-pyrazol-4-yl)pyridin-4-yl)piperidin-3-ol;
4-((S)-3-aminopiperidin-1-yl)-N-(6-(2-fluoro-6-methoxyphenyl)-5-nitropyridin-2-yl-) 5-(1-(tetrahydro-2H-pyran-4-yl)-1H-pyrazol-4-yl)pyridin-2-amine;
methyl 6-((4-((S)-3-aminopiperidin-1-yl)-5-(1-(tetrahydro-2H-pyran-4-yl)-1H-pyrazol-4-yl)pyridin-2-yl)amino)-2-(2-fluoro-6-methoxyphenyl)nicotinate;
(S)-1-(5-(1-(difluoromethyl)-1H-pyrazol-4-yl)-2-((2-(2-fluoro-6-methoxyphenyl)pyrimidin-4-yl)amino)pyridin-4-yl)piperidin-3-ol;
(S)-N-(4-(3-aminopiperidin-1-yl)-5-(1-(difluoromethyl)-1H-pyrazol-4-yl)pyridin-2-yl)-2-(2-fluoro-6-methoxyphenyl)pyrimidin-4-amine;
(3S)-1-(2-((5-chloro-6-(2-fluoro-6-methoxyphenyl)pyridin-2-yl)amino)-5-(1-(tetrahydro-2H-pyran-4-yl)-1H-pyrazol-4-yl)pyridin-4-yl)piperidin-3-ol;
4-((S)-3-aminopiperidin-1-yl)-5-(1-(difluoromethyl)-1H-pyrazol-4-yl)-N-(5-fluoro-6-(2-fluoro-6-methoxyphenyl)pyridin-2-yl)pyridin-2-amine;
(R)-N-(4-(3-aminopiperidin-1-yl)-5-(1-(2,2,2-trifluoroethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(2,2,2-trifluoroethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-((tetrahydro-2H-pyran-4-yl)ethynyl)pyridin-2-yl)-2-(2-fluoro-6-methoxyphenyl)pyrimidin-4-amine;
2-(2-fluoro-6-methoxyphenyl)-N-(4-(3-(fluoromethyl)piperidin-1-yl)-5-(1-(tetrahydro-2H-pyran-4-yl)-1H-pyrazol-4-yl)pyridin-2-yl)pyrimidin-4-amine;
(S)-N-(4-(3-aminopiperidin-1-yl)-5-(1-(2,2,2-trifluoroethyl)-1H-pyrazol-4-yl)pyridin-2-yl)-2-(7-fluoroquinoxalin-6-yl)pyrimidin-4-amine;
(S)-N-(4-(3-aminopiperidin-1-yl)-5-(1-(2-methoxyethyl)-1H-pyrazol-4-yl)pyridin-2-yl)-2-(2-fluoro-6-methoxyphenyl)pyrimidin-4-amine;
(3R,4R)-4-fluoro-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-ol;
(3R,4R)-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)-4-(hydroxymethyl)piperidin-3-ol;
(S)-N-(4-(3-aminopiperidin-1-yl)-5-(1-(trifluoromethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(difluoromethyl)-1H-pyrazol-4-yl)pyridin-2-yl)-2-(2,4-difluoro-6-methoxyphenyl)pyrimidin-4-amine;
(S)-N-(4-(3-aminopiperidin-1-yl)-5-(1-(2,2,2-trifluoroethyl)-1H-pyrazol-4-yl)pyridin-2-yl)-2-(2,4-difluoro-6-methoxyphenyl)pyrimidin-4-amine;
(S)-N-(4-(3-aminopiperidin-1-yl)-5-((1-(trifluoromethyl)-1H-pyrazol-4-yl)ethynyl)pyridin-2-yl)-2-(2-fluoro-6-methoxyphenyl)pyrimidin-4-amine;
2-(2-fluoro-6-methoxyphenyl)-6-((4-((S)-3-hydroxypiperidin-1-yl)-5-(1-(tetrahydro-2H-pyran-4-yl)-1H-pyrazol-4-yl)pyridin-2-yl)amino)nicotinamide;
(S)-N-(4-(3-aminopiperidin-1-yl)-5-(1-(3-methoxypropyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(2,2-difluoroethyl)-1H-pyrazol-4-yl)pyridin-2-yl)-2-(2-fluoro-6-methoxyphenyl)pyrimidin-4-amine;
N-(5-(1-(difluoromethyl)-1H-pyrazol-4-yl)-4-(2-methyl-2,8-diazaspiro[4.5]decan-8-yl)pyridin-2-yl)-2-(2-fluoro-6-methoxyphenyl)pyrimidin-4-amine;
2-(2-fluoro-6-methoxyphenyl)-N-(4-(2-methyl-2,8-diazaspiro[4.5]decan-8-yl)-5-(1-(2,2,2-trifluoroethyl)-1H-pyrazol-4-yl)pyridin-2-yl)pyrimidin-4-amine;
2-(2-fluoro-6-methoxyphenyl)-6-((4-((S)-3-hydroxypiperidin-1-yl)-5-((tetrahydro-2H-pyran-4-yl)ethynyl)pyridin-2-yl)amino)nicotinamide;
6-((5-(1-(difluoromethyl)-1H-pyrazol-4-yl)-4-((S)-3-hydroxypiperidin-1-yl)pyridin-2-yl)amino)-2-(2-fluoro-6-methoxyphenyl)nicotinamide;
2-(2-fluoro-6-methoxyphenyl)-6-((4-((S)-3-hydroxypiperidin-1-yl)-5-(1-(2,2,2-tri fluoroethyl)-1H-pyrazol-4-yl)pyridin-2-yl)amino)nicotinamide;
(3S)-1-(5-(1-(difluoromethyl)-1H-pyrazol-4-yl)-2-((6-(2-fluoro-6-methoxyphenyl)-5-nitropyridin-2-yl)amino)pyridin-4-yl)piperidin-3-ol;
4-((S)-3-aminopiperidin-1-yl)-5-(1-(difluoromethyl)-1H-pyrazol-4-yl)-N-(6-(2-fluoro-6-methoxyphenyl)-5-nitropyridin-2-yl)pyridin-2-amine;
(3S)-1-(2-((5-amino-6-(2-fluoro-6-methoxyphenyl)pyridin-2-yl)amino)-5-(1-(difluoromethyl)-1H-pyrazol-4-yl)pyridin-4-yl)piperidin-3-ol;
N²-(4-((S)-3-aminopiperidin-1-yl)-5-(1-(difluoromethyl)-1H-pyrazol-4-yl)pyridin-2-yl)-6-(2-fluoro-6-methoxyphenyl)pyridin-2,5-diamine;
4-((S)-3-aminopiperidin-1-yl)-5-(1-(2,2-difluoroethyl)-1H-pyrazol-4-yl)-N-(6-(2-fluoro-6-methoxyphenyl)-5-nitropyridin-2-yl)pyridin-2-amine;
4-((S)-3-aminopiperidin-1-yl)-N-(6-(2-fluoro-6-methoxyphenyl)-5-nitropyridin-2-yl-) 5-(1-(2,2,2-trifluoroethyl)-1H-pyrazol-4-yl)pyridin-2-amine;
4-((S)-3-aminopiperidin-1-yl)-N-(6-(2-fluoro-6-methoxyphenyl)-5-nitropyridin-2-yl-) 5-(1-(trifluoromethyl)-1H-pyrazol-4-yl)pyridin-2-amine;
4-((S)-3-aminopiperidin-1-yl)-N-(6-(2-fluoro-6-methoxyphenyl)-5-nitropyridin-2-yl-) 5-((tetrahydro-2H-pyran-4-yl)ethynyl)pyridin-2-amine;
(S)-N-(4-(3-aminopiperidin-1-yl)-5-(1-(trifluoromethyl)-1H-pyrazol-4-yl)pyridin-2-yl)-2-(2,4-difluoro-6-methoxyphenyl)pyrimidin-4-amine;
(S)-N-(4-(3-aminopiperidin-1-yl)-5-(1-(2,2-difluoroethyl)-1H-pyrazol-4-yl)pyridin-2-yl)-2-(2,4-difluoro-6-methoxyphenyl)pyrimidin-4-amine;
(S)-1-(2-((2-(2-fluoro-4-(hydroxymethyl)-6-methoxyphenyl)pyrimidin-4-yl)amino)-5-(1-(tetrahydro-2H-pyran-4-yl)-1H-pyrazol-4-yl)pyridin-4-yl)piperidin-3-ol;
(S)-N-(4-(3-aminopiperidin-1-yl)-5-((1-(difluoromethyl)-1H-pyrazol-4-yl)ethynyl)pyridin-2-yl)-2-(2-fluoro-6-methoxyphenyl)pyrimidin-4-amine;
(S)-N-(4-(3-aminopiperidin-1-yl)-5-((1-(2,2-difluoroethyl)-1H-pyrazol-4-yl)ethynyl)pyridin-2-yl)-2-(2-fluoro-6-methoxyphenyl)pyrimidin-4-amine;
(S)-N-(4-(3-aminopiperidin-1-yl)-5-((1-(2,2,2-trifluoroethyl)-1H-pyrazol-4-yl)ethynyl)pyridin-2-yl)-2-(2-fluoro-6-methoxyphenyl)pyrimidin-4-amine;
(S)-1-(2-((2-(2,4-difluoro-6-methoxyphenyl)pyrimidin-4-yl)amino)-5-(1-(trifluoromethyl)-1H-pyrazol-4-yl)pyridin-4-yl)piperidin-3-ol;
(S)-1-(2-((2-(2,4-difluoro-6-methoxyphenyl)pyrimidin-4-yl)amino)-5-(1-(2,2-difluoroethyl)-1H-pyrazol-4-yl)pyridin-4-yl)piperidin-3-ol;
N²-(4-((S)-3-aminopiperidin-1-yl)-5-(1-(trifluoromethyl)-1H-pyrazol-4-yl)pyridin-2-yl)-6-(2-fluoro-6-methoxyphenyl)pyridin-2,5-diamine;
(S)-1-(2-((2-(2,4-difluoro-6-methoxyphenyl)pyrimidin-4-yl)amino)-5-(1-(2,2,2-tri fluoroethyl)-1H-pyrazol-4-yl)pyridin-4-yl)piperidin-3-ol;
(3S)-1-(2-((5-amino-6-(2-fluoro-6-methoxyphenyl)pyridin-2-yl)amino)-5-(1-(tetrahydro-2H-pyran-4-yl)-1H-pyrazol-4-yl)pyridin-4-yl)piperidin-3-ol;
(3S)-1-(2-((5-amino-6-(2-fluoro-6-methoxyphenyl)pyridin-2-yl)amino)-5-(1-(trifluoromethyl)-1H-pyrazol-4-yl)pyridin-4-yl)piperidin-3-ol;
(3S)-1-(2-((5-amino-6-(2-fluoro-6-methoxyphenyl)pyridin-2-yl)amino)-5-(1-(2,2-di fluoroethyl)-1H-pyrazol-4-yl)pyridin-4-yl)piperidin-3-ol;
(3S)-1-(2-((5-amino-6-(2-fluoro-6-methoxyphenyl)pyridin-2-yl)amino)-5-(1-(2,2,2-trifluoroethyl)-1H-pyrazol-4-yl)pyridin-4-yl)piperidin-3-ol;
(S)-1-(2-((2-(2,4-difluoro-6-methoxyphenyl)pyrimidin-4-yl)amino)-5-(1-(difluoromethyl)-1H-pyrazol-4-yl)pyridin-4-yl)piperidin-3-ol;
(3S)-1-(2-((6-(2,4-difluoro-6-methoxyphenyl)-5-nitropyridin-2-yl)amino)-5-(1-(di fluoromethyl)-1H-pyrazol-4-yl)pyridin-4-yl)piperidin-3-ol;
(3S)-1-(2-((5-amino-6-(2,4-difluoro-6-methoxyphenyl)pyridin-2-yl)amino)-5-(1-(di fluoromethyl)-1H-pyrazol-4-yl)pyridin-4-yl)piperidin-3-ol;
methyl (S)-4-(4-((4-(3-aminopiperidin-1-yl)-5-(1-(difluoromethyl)-1H-pyrazol-4-yl)pyridin-2-yl)amino)pyrimidin-2-yl)-3-fluoro-5-methoxybenzoate;
(S)-4-(4-((4-(3-aminopiperidin-1-yl)-5-(1-(difluoromethyl)-1H-pyrazol-4-yl)pyridin-2-yl)amino)pyrimidin-2-yl)-3-fluoro-5-methoxy-N,N-dimethylbenzamide;
(S)-3-fluoro-4-(4-((4-(3-hydroxypiperidin-1-yl)-5-(1-(tetrahydro-2H-pyran-4-yl)-1H-pyrazol-4-yl)pyridin-2-yl)amino)pyrimidin-2-yl)-5-methoxybenzaldehyde;
6-((6-(2-fluoro-6-methoxyphenyl)-5-nitropyridin-2-yl)amino)-N-((1R,2R)-2-hydroxycyclobutyl)-4-((S)-3-hydroxypiperidin-1-yl)nicotinamide;
N-(4-((3S,4R)-3-amino-4-methylpiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3S,5S)-3-amino-5-methylpiperidin-1-yl)-5-(1-(difluoromethyl)-1H-pyrazol-4-yl)pyridin-2-yl)-2-(2-fluoro-6-methoxyphenyl)pyrimidin-4-amine;
(S)-N-(4-(3-amino-3-methylpiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3R,4S)-3-amino-4-fluoropiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3R,4R)-3-amino-4-fluoropiperidin-1-yl)-5-(1-(difluoromethyl)-1H-pyrazol-4-yl)pyridin-2-yl)-2-(2-fluoro-6-methoxyphenyl)pyrimidin-4-amine;
(3R,4R)-3-amino-1-(5-(1-(difluoromethyl)-1H-pyrazol-4-yl)-2-((2-(2-fluoro-6-methoxyphenyl)pyrimidin-4-yl)amino)pyridin-4-yl)piperidin-4-ol;
N-(4-((3S,4R)-3-amino-4-methylpiperidin-1-yl)-5-(1-(difluoromethyl)-1H-pyrazol-4-yl)pyridin-2-yl)-2-(2,4-difluoro-6-methoxyphenyl)pyrimidin-4-amine;
(3R,4R)-3-amino-1-(2-((2-(2,4-difluoro-6-methoxyphenyl)pyrimidin-4-yl)amino)-5-(1-(difluoromethyl)-1H-pyrazol-4-yl)pyridin-4-yl)piperidin-4-ol;
(1S,4S)-4-((5-(1-(difluoromethyl)-1H-pyrazol-4-yl)-2-((2-(2-fluoro-6-methoxyphenyl)pyrimidin-4-yl)amino)pyridin-4-yl)(methyl)amino)cyclohexan-1-ol;
N²-(4-((3S,4R)-3-amino-4-methylpiperidin-1-yl)-5-(1-(difluoromethyl)-1H-pyrazol-4-yl)pyridin-2-yl)-6-(2,4-difluoro-6-methoxyphenyl)pyridin-2,5-diamine;
N²-(4-((3R,4R)-3-amino-4-fluoropiperidin-1-yl)-5-(1-(difluoromethyl)-1H-pyrazol-4-yl)pyridin-2-yl)-6-(2,4-difluoro-6-methoxyphenyl)pyridin-2,5-diamine;
(S)-1-(2-((2-(2,4-difluoro-6-methoxyphenyl)pyrimidin-4-yl)amino)-5-((1-(difluoromethyl)-1H-pyrazol-4-yl)ethynyl)pyridin-4-yl)piperidin-3-ol;
(S)-1-(2-((2-(2,4-difluoro-6-methoxyphenyl)pyrimidin-4-yl)amino)-5-((1-(2,2-difluoroethyl)-1H-pyrazol-4-yl)ethynyl)pyridin-4-yl)piperidin-3-ol;
(3S)-1-(2-((6-(2,4-difluoro-6-methoxyphenyl)-5-nitropyridin-2-yl)amino)-5-(1-(2,2-difluoroethyl)-1H-pyrazol-4-yl)pyridin-4-yl)piperidin-3-ol;
(3S)-1-(2-((6-(2,4-difluoro-6-methoxyphenyl)-5-nitropyridin-2-yl)amino)-5-(1-(tetrahydro-2H-pyran-4-yl)-1H-pyrazol-4-yl)pyridin-4-yl)piperidin-3-ol;
(3S)-1-(2-((5-amino-6-(2,4-difluoro-6-methoxyphenyl)pyridin-2-yl)amino)-5-(1-(2,2-difluoroethyl)-1H-pyrazol-4-yl)pyridin-4-yl)piperidin-3-ol;
(3S)-1-(2-((5-amino-6-(2,4-difluoro-6-methoxyphenyl)pyridin-2-yl)amino)-5-(1-(tetrahydro-2H-pyran-4-yl)-1H-pyrazol-4-yl)pyridin-4-yl)piperidin-3-ol;
(S)-N-(4-(3-aminopiperidin-1-yl)-5-(1-(2-methoxy-2-methylpropyl)-1H-pyrazol-4-yl)pyridin-2-yl)-2-(2-fluoro-6-methoxyphenyl)pyrimidin-4-amine;
(S)-4-(3-aminopiperidin-1-yl)-N-(2-(2-fluoro-6-methoxyphenyl)pyrimidin-4-yl)-6′-(2-methoxyethoxy)-[3,3′-bipyridin]-6-amine;
(S)-(3,3-difluoroazetidin-1-yl)(4-(4-((5-(1-(difluoromethyl)-1H-pyrazol-4-yl)-4-(3-hydroxypiperidin-1-yl)pyridin-2-yl)amino)pyrimidin-2-yl)-3-fluoro-5-methoxyphenyl)methanone;
(4-(4-((5-(1-(difluoromethyl)-1H-pyrazol-4-yl)-4-((S)-3-hydroxypiperidin-1-yl)pyridin-2-yl)amino)pyrimidin-2-yl)-3-fluoro-5-methoxyphenyl)((R)-3-fluoropyrrolidin-1-yl)methanone;
(S)-(4-(4-((5-(1-(difluoromethyl)-1H-pyrazol-4-yl)-4-(3-hydroxypiperidin-1-yl)pyridin-2-yl)amino)pyrimidin-2-yl)-3-fluoro-5-methoxyphenyl)(3,3-difluoropyrrolidin-1-yl)methanone;
(S)-1-(4-(4-(3-aminopiperidin-1-yl)-6-((2-(2-fluoro-6-methoxyphenyl)pyrimidin-4-yl)amino)pyridin-3-yl)-1H-pyrazol-1-yl)-2-methylpropan-2-ol;
(S)-N-(4-(3-aminopiperidin-1-yl)-5-(1-(1-methoxy-2-methylpropan-2-yl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-((3,3-difluorocyclobutyl)methyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(fluoromethyl)-1H-pyrazol-4-yl)pyridin-2-yl)-2-(2-fluoro-6-methoxyphenyl)pyrimidin-4-amine;
N-(4-((2R,5S)-5-amino-2-methylpiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3R,4S)-3-amino-4-methoxypiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3R,4R)-3-amino-4-methoxypiperidin-1-yl)-5-(1-(difluoromethyl)-1H-pyrazol-4-yl)pyridin-2-yl)-2-(2-fluoro-6-methoxyphenyl)pyrimidin-4-amine;
(S)-7-(4-(3-aminopiperidin-1-yl)-6-((2-(2-fluoro-6-methoxyphenyl)pyrimidin-4-yl)amino)pyridin-3-yl)-4-methyl-2H-benzo[b][1,4]oxazin-3(4H)-one;
(S)-6-(4-(3-aminopiperidin-1-yl)-6-((2-(2-fluoro-6-methoxyphenyl)pyrimidin-4-yl)amino)pyridin-3-yl)-8-methyl-3,4-dihydroquinolin-2(1H)-one;
(S)-6-(4-(3-aminopiperidin-1-yl)-6-((2-(2-fluoro-6-methoxyphenyl)pyrimidin-4-yl)amino)pyridin-3-yl)-1-methyl-3,4-dihydroquinolin-2(1H)-one;
(S)-1-(2-((2-(2-fluoro-6-methoxyphenyl)pyrimidin-4-yl)amino)-5-(1-(2,2,6,6-tetramethyltetrahydro-2H-pyran-4-yl)-1H-pyrazol-4-yl)pyridin-4-yl)piperidin-3-ol;
(S)-N-(5-(1-(difluoromethyl)-1H-pyrazol-4-yl)-4-(3-((methylamino)methyl)piperidin-1-yl)pyridin-2-yl)-2-(2-fluoro-6-methoxyphenyl)pyrimidin-4-amine;
(R)-N-(5-(1-(difluoromethyl)-1H-pyrazol-4-yl)-4-(3-((methylamino)methyl)piperidin-1-yl)pyridin-2-yl)-2-(2-fluoro-6-methoxyphenyl)pyrimidin-4-amine;
N-(4-((3R,4R)-4-amino-3-methylpiperidin-1-yl)-5-(1-(difluoromethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(2,2,6,6-tetramethyltetrahydro-2H-pyran-4-yl)-1H-pyrazol-4-yl)pyridin-2-yl)-2-(2-fluoro-6-methoxyphenyl)pyrimidin-4-amine;
(S)-N-(5-(1-(difluoromethyl)-1H-pyrazol-4-yl)-4-(3-(fluoromethyl)piperidin-1-yl)pyridin-2-yl)-2-(2-fluoro-6-methoxyphenyl)pyrimidin-4-amine;
(R)-N-(5-(1-(difluoromethyl)-1H-pyrazol-4-yl)-4-(3-(difluoromethyl)piperidin-1-yl)pyridin-2-yl)-2-(2-fluoro-6-methoxyphenyl)pyrimidin-4-amine;
methyl (S)-2-(4-(4-(3-aminopiperidin-1-yl)-6-((2-(2-fluoro-6-methoxyphenyl)pyrimidin-4-yl)amino)pyridin-3-yl)-1H-pyrazol-1-yl)-2-methylpropanoate;
(S)-N-(4-(3-aminopiperidin-1-yl)-5-(4-methoxy-3,5-dimethylphenyl)pyridin-2-yl)-2-(2-fluoro-6-methoxyphenyl)pyrimidin-4-amine;
(S)-N-(4-(3-aminopiperidin-1-yl)-5-(4-morpholinophenyl)pyridin-2-yl)-2-(2-fluoro-6-methoxyphenyl)pyrimidin-4-amine;
(S)-N-(4-(3-aminopiperidin-1-yl)-5-(3-methyl-4-morpholinophenyl)pyridin-2-yl)-2-(2-fluoro-6-methoxyphenyl)pyrimidin-4-amine;
(S)-4-(4-(3-aminopiperidin-1-yl)-6-((2-(2-fluoro-6-methoxyphenyl)pyrimidin-4-yl)amino)pyridin-3-yl)-2-fluoro-N,N-dimethylbenzamide;
(S)-1-(3-(4-(3-aminopiperidin-1-yl)-6-((2-(2-fluoro-6-methoxyphenyl)pyrimidin-4-yl)amino)pyridin-3-yl)phenyl)ethan-1-one;
(S)-N-(4-(3-aminopiperidin-1-yl)-5-(3,4-difluorophenyl)pyridin-2-yl)-2-(2-fluoro-6-methoxyphenyl)pyrimidin-4-amine;
(3R,4R)-4-amino-1-(5-(1-(difluoromethyl)-1H-pyrazol-4-yl)-2-((2-(2-fluoro-6-methoxyphenyl)pyrimidin-4-yl)amino)pyridin-4-yl)piperidin-3-ol;
N-(4-((3R,4S)-4-amino-3-methylpiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((S)-3-aminopiperidin-1-yl)-5-(1-((S)-tetrahydrofuran-3-yl)-1H-pyrazol-4-yl)pyridin-2-yl)-2-(2-fluoro-6-methoxyphenyl)pyrimidin-4-amine;
N-(4-((S)-3-aminopiperidin-1-yl)-5-(1-((R)-tetrahydrofuran-3-yl)-1H-pyrazol-4-yl)pyridin-2-yl)-2-(2-fluoro-6-methoxyphenyl)pyrimidin-4-amine;
6-((4-((S)-3-aminopiperidin-1-yl)-5-(1-(tetrahydro-2H-pyran-4-yl)-1H-pyrazol-4-yl)pyridin-2-yl)amino)-2-(2-fluoro-6-methoxyphenyl)nicotinonitrile;
6-((4-((S)-3-aminopiperidin-1-yl)-5-(1-(difluoromethyl)-1H-pyrazol-4-yl)pyridin-2-yl)amino)-2-(2-fluoro-6-methoxyphenyl)nicotinonitrile;
N-(4-((3S,5S)-3-amino-5-fluoropiperidin-1-yl)-5-(1-(difluoromethyl)-1H-pyrazol-4-yl)pyridin-2-yl)-2-(2-fluoro-6-methoxyphenyl)pyrimidin-4-amine;
6-((4-((S)-3-aminopiperidin-1-yl)-5-(1-(difluoromethyl)-1H-pyrazol-4-yl)pyridin-2-yl)amino)-2-(2,4-difluoro-6-methoxyphenyl)nicotinonitrile;
(S)-4-(4-(3-aminopiperidin-1-yl)-6-((2-(2,4-difluoro-6-methoxyphenyl)pyrimidin-4-yl)amino)pyridin-3-yl)-2-methylbut-3-yn-2-ol;
6-((4-((S)-3-aminopiperidin-1-yl)-5-(1-(2,2,2-trifluoroethyl)-1H-pyrazol-4-yl)pyridin-2-yl)amino)-2-(2,4-difluoro-6-methoxyphenyl)nicotinonitrile;
6-((4-((3R,4S)-3-amino-4-fluoropiperidin-1-yl)-5-(1-(difluoromethyl)-1H-pyrazol-4-yl)pyridin-2-yl)amino)-2-(2-fluoro-6-methoxyphenyl)nicotinonitrile; and
6-((4-((3R,4R)-4-amino-3-hydroxypiperidin-1-yl)-5-(1-(difluoromethyl)-1H-pyrazol-4-yl)pyridin-2-yl)amino)-2-(2-fluoro-6-methoxyphenyl)nicotinonitrile.

In the compound of Formula 1 or pharmaceutically acceptable salt, still more preferable compounds include the following compound or pharmaceutically acceptable salt thereof selected from the group consisting of:
(S)-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-ol;
(S)-1-(6-((2-(2-fluoro-6-methoxyphenyl)pyrimidin-4-yl)amino)-6′-(tetrahydro-2H-pyran-4-yl)-[3,3′-bipyridin]-4-yl)piperidin-3-ol;
(3S)-1-(2-((5-fluoro-6-(2-fluoro-6-methoxyphenyl)pyridin-2-yl)amino)-5-(1-(tetrahydro-2H-pyran-4-yl)-1H-pyrazol-4-yl)pyridin-4-yl)piperidin-3-ol;
(3S)-1-(2-((6-(2-fluoro-6-methoxyphenyl)-5-nitropyridin-2-yl)amino)-5-(1-(tetrahydro-2H-pyran-4-yl)-1H-pyrazol-4-yl)pyridin-4-yl)piperidin-3-ol;
(S)-N-(4-(3-aminopiperidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
methyl 2-(2-fluoro-6-methoxyphenyl)-6-((4-((S)-3-hydroxypiperidin-1-yl)-5-(1-(tetrahydro-2H-pyran-4-yl)-1H-pyrazol-4-yl)pyridin-2-yl)amino)nicotinate;
(S)-1-(2-((2-(7-fluoroquinoxalin-6-yl)pyrimidin-4-yl)amino)-5-(1-(tetrahydro-2H-pyran-4-yl)-1H-pyrazol-4-yl)pyridin-4-yl)piperidin-3-ol;
4-((S)-3-aminopiperidin-1-yl)-N-(6-(2-fluoro-6-methoxyphenyl)-5-nitropyridin-2-yl)-5-(1-(tetrahydro-2H-pyran-4-yl)-1H-pyrazol-4-yl)pyridin-2-amine;
(S)-1-(5-(1-(difluoromethyl)-1H-pyrazol-4-yl)-2-((2-(2-fluoro-6-methoxyphenyl)pyrimidin-4-yl)amino)pyridin-4-yl)piperidin-3-ol;
(S)-N-(4-(3-aminopiperidin-1-yl)-5-(1-(difluoromethyl)-1H-pyrazol-4-yl)pyridin-2-yl)-2-(2-fluoro-6-methoxyphenyl)pyrimidin-4-amine;
4-((S)-3-aminopiperidin-1-yl)-5-(1-(difluoromethyl)-1H-pyrazol-4-yl)-N-(5-fluoro-6-(2-fluoro-6-methoxyphenyl)pyridin-2-yl)pyridin-2-amine;
(S)-N-(4-(3-aminopiperidin-1-yl)-5-(1-(2,2,2-trifluoroethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-((tetrahydro-2H-pyran-4-yl)ethynyl)pyridin-2-yl)-2-(2-fluoro-6-methoxyphenyl)pyrimidin-4-amine;
(S)-N-(4-(3-aminopiperidin-1-yl)-5-(1-(2-methoxyethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(trifluoromethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(difluoromethyl)-1H-pyrazol-4-yl)pyridin-2-yl)-2-(2,4-difluoro-6-methoxyphenyl)pyrimidin-4-amine;
(S)-N-(4-(3-aminopiperidin-1-yl)-5-(1-(2,2,2-trifluoroethyl)-1H-pyrazol-4-yl)pyridin-2-yl)-2-(2,4-difluoro-6-methoxyphenyl)pyrimidin-4-amine;
2-(2-fluoro-6-methoxyphenyl)-6-((4-((S)-3-hydroxypiperidin-1-yl)-5-(1-(tetrahydro-2H-pyran-4-yl)-1H-pyrazol-4-yl)pyridin-2-yl)amino)nicotinamide;
(S)-N-(4-(3-aminopiperidin-1-yl)-5-(1-(3-methoxypropyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(2,2-difluoroethyl)-1H-pyrazol-4-yl)pyridin-2-yl)-2-(2-fluoro-6-methoxyphenyl)pyrimidin-4-amine;
6-((5-(1-(difluoromethyl)-1H-pyrazol-4-yl)-4-((S)-3-hydroxypiperidin-1-yl)pyridin-2-yl)amino)-2-(2-fluoro-6-methoxyphenyl)nicotinamide;
4-((S)-3-aminopiperidin-1-yl)-5-(1-(difluoromethyl)-1H-pyrazol-4-yl)-N-(6-(2-fluoro-6-methoxyphenyl)-5-nitropyridin-2-yl)pyridin-2-amine;
(3S)-1-(2-((5-amino-6-(2-fluoro-6-methoxyphenyl)pyridin-2-yl)amino)-5-(1-(difluoromethyl)-1H-pyrazol-4-yl)pyridin-4-yl)piperidin-3-ol;
4-((S)-3-aminopiperidin-1-yl)-5-(1-(2,2-difluoroethyl)-1H-pyrazol-4-yl)-N-(6-(2-fluoro-6-methoxyphenyl)-5-nitropyridin-2-yl)pyridin-2-amine;
4-((S)-3-aminopiperidin-1-yl)-N-(6-(2-fluoro-6-methoxyphenyl)-5-nitropyridin-2-yl)-5-(1-(trifluoromethyl)-1H-pyrazol-4-yl)pyridin-2-amine;
4-((S)-3-aminopiperidin-1-yl)-N-(6-(2-fluoro-6-methoxyphenyl)-5-nitropyridin-2-yl)-5-((tetrahydro-2H-pyran-4-yl)ethynyl)pyridin-2-amine;
(S)-N-(4-(3-aminopiperidin-1-yl)-5-(1-(2,2-difluoroethyl)-1H-pyrazol-4-yl)pyridin-2-yl)-2-(2,4-difluoro-6-methoxyphenyl)pyrimidin-4-amine;
(S)-N-(4-(3-aminopiperidin-1-yl)-5-((1-(difluoromethyl)-1H-pyrazol-4-yl)ethynyl)pyridin-2-yl)-2-(2-fluoro-6-methoxyphenyl)pyrimidin-4-amine;
N²-(4-((S)-3-aminopiperidin-1-yl)-5-(1-(trifluoromethyl)-1H-pyrazol-4-yl)pyridin-2-yl)-6-(2-fluoro-6-methoxyphenyl)pyridin-2,5-diamine;
(S)-4-(4-((4-(3-aminopiperidin-1-yl)-5-(1-(difluoromethyl)-1H-pyrazol-4-yl)pyridin-2-yl)amino)pyrimidin-2-yl)-3-fluoro-5-methoxy-N,N-dimethylbenzamide;
(3R,4R)-3-amino-1-(5-(1-(difluoromethyl)-1H-pyrazol-4-yl)-2-((2-(2-fluoro-6-methoxyphenyl)pyrimidin-4-yl)amino)pyridin-4-yl)piperidin-4-ol;
N-(4-((3S,4R)-3-amino-4-methylpiperidin-1-yl)-5-(1-(difluoromethyl)-1H-pyrazol-4-yl)pyridin-2-yl)-2-(2,4-difluoro-6-methoxyphenyl)pyrimidin-4-amine;
(3R,4R)-3-amino-1-(2-((2-(2,4-difluoro-6-methoxyphenyl)pyrimidin-4-yl)amino)-5-(1-(difluoromethyl)-1H-pyrazol-4-yl)pyridin-4-yl)piperidin-4-ol;
N²-(4-((3S,4R)-3-amino-4-methylpiperidin-1-yl)-5-(1-(difluoromethyl)-1H-pyrazol-4-yl)pyridin-2-yl)-6-(2,4-difluoro-6-methoxyphenyl)pyridin-2,5-diamine;
N²-(4-((3R,4R)-3-amino-4-fluoropiperidin-1-yl)-5-(1-(difluoromethyl)-1H-pyrazol-4-yl)pyridin-2-yl)-6-(2,4-difluoro-6-methoxyphenyl)pyridin-2,5-diamine;
(S)-1-(4-(4-(3-aminopiperidin-1-yl)-6-((2-(2-fluoro-6-methoxyphenyl)pyrimidin-4-yl)amino)pyridin-3-yl)-1H-pyrazol-1-yl)-2-methylpropan-2-ol;
(S)-N-(4-(3-aminopiperidin-1-yl)-5-(1-(fluoromethyl)-1H-pyrazol-4-yl)pyridin-2-yl)-2-(2-fluoro-6-methoxyphenyl)pyrimidin-4-amine;
N-(4-((3R,4R)-4-amino-3-methylpiperidin-1-yl)-5-(1-(difluoromethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(2,2,6,6-tetramethyltetrahydro-2H-pyran-4-yl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(3,4-difluorophenyl)pyridin-2-yl)-2-(2-fluoro-6-methoxyphenyl)pyrimidin-4-amine;
(3R,4R)-4-amino-1-(5-(1-(difluoromethyl)-1H-pyrazol-4-yl)-2-((2-(2-fluoro-6-methoxyphenyl)pyrimidin-4-yl)amino)pyridin-4-yl)piperidin-3-ol;
N-(4-((3R,4S)-4-amino-3-methylpiperidin-1-yl)-5-(1-(difluoromethyl)-1H-pyrazol-4-yl)pyridin-2-yl)-2-(2-fluoro-6-methoxyphenyl)pyrimidin-4-amine;
6-((4-((S)-3-aminopiperidin-1-yl)-5-(1-(difluoromethyl)-1H-pyrazol-4-yl)pyridin-2-yl)amino)-2-(2-fluoro-6-methoxyphenyl)nicotinonitrile;
N-(4-((3S,5S)-3-amino-5-fluoropiperidin-1-yl)-5-(1-(difluoromethyl)-1H-pyrazol-4-yl)pyridin-2-yl)-2-(2-fluoro-6-methoxyphenyl)pyrimidin-4-amine;
6-((4-((S)-3-aminopiperidin-1-yl)-5-(1-(difluoromethyl)-1H-pyrazol-4-yl)pyridin-2-yl)amino)-2-(2,4-difluoro-6-methoxyphenyl)nicotinonitrile;
(S)-4-(4-(3-aminopiperidin-1-yl)-6-((2-(2,4-difluoro-6-methoxyphenyl)pyrimidin-4-yl)amino)pyridin-3-yl)-2-methylbut-3-yn-2-ol;
6-((4-((S)-3-aminopiperidin-1-yl)-5-(1-(2,2,2-trifluoroethyl)-1H-pyrazol-4-yl)pyridin-2-yl)amino)-2-(2,4-difluoro-6-methoxyphenyl)nicotinonitrile;
6-((4-((3R,4S)-3-amino-4-fluoropiperidin-1-yl)-5-(1-(difluoromethyl)-1H-pyrazol-4-yl)pyridin-2-yl)amino)-2-(2-fluoro-6-methoxyphenyl)nicotinonitrile; and
6-((4-((3R,4R)-4-amino-3-hydroxypiperidin-1-yl)-5-(1-(difluoromethyl)-1H-pyrazol-4-yl)pyridin-2-yl)amino)-2-(2-fluoro-6-methoxyphenyl)nicotinonitrile.

The compound of Formula 1 or pharmaceutically acceptable salt thereof may be in the form of structural isomer such as tautomer and in the form of stereoisomer such as R or S isomer having with asymmetric carbon center(s), cis- or trans-geometrical isomer, or optical isomer (enantiomer). Therefore, the compound of Formula 1 or pharmaceutically acceptable salt thereof includes said all forms, such as a cis- or trans-geometrical isomer, a (R)- or (S)-optical isomer, and a racemic mixture (RS), unless otherwise indicated.

The compound of Formula 1 of the present invention may be in a pharmaceutically acceptable salt form. The salt may be a conventional acid addition salt form, which includes e.g., salts derived from an inorganic acid such as hydrochloric acid, sulfuric acid, nitric acid, phosphoric acid, perchloric acid, or hydrobromic acid; and salts derived from an organic acid such as formic acid, acetic acid, propionic acid, oxalic acid, succinic acid, benzoic acid, citric acid, maleic acid, malonic acid, malic acid, tartaric acid, gluconic acid, lactic acid, gentisic acid, fumaric acid, lactobionic acid, salicylic acid, phthalic acid, embonic acid, aspartic acid, glutamic acid, or acetylsalicylic acid. And, the salt includes a salt derived from amino acids such as glycine, alanine, valine, isoleucine, serine, cysteine, cystine, aspartic acid, glutamine, lysine, arginine, tyrosine, and proline. In addition, the salt includes a salt derived from sulfonic acids, such as methanesulfonic acid, ethanesulfonic acid, benzenesulfonic acid, or toluenesulfonic acid.

The present invention includes, within its scope, a process for preparing the compound of Formula 1 or pharmaceutically acceptable salt thereof.

For example, the compound of Formula 1 or pharmaceutically acceptable salt thereof according to the present invention may be prepared by a process which comprises preparing a compound of Formula IV from a compound of Formula II, via a compound of Formula III-a or III-b, through Synthesis 1 or Synthesis 2; and reacting the compound of Formula IV with a compound of Formula V through Synthesis 3 to prepare a compound of Formula 1, as shown in the following Reaction Scheme 1:

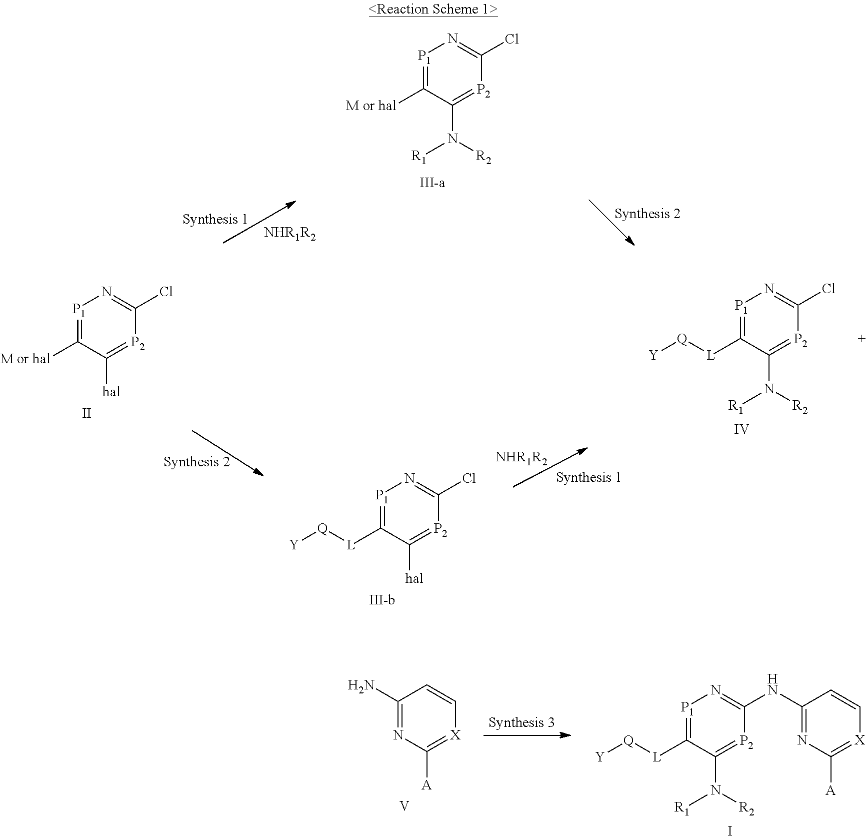

In the Reaction Scheme 1, hal is halogen; M is —B(OH)₂ or pinacolborane (-BPin); and P₁, P₂, R₁, R₂, L, Q, Y, X, and A are the same as defined in the above.

In the Reaction Scheme 1, the reaction of Synthesis 1 may be carried out by reacting the commercially available compound of Formula II or III-b with NHR₁R₂. The reaction may be carried out with a base such as cesium carbonate (Cs₂CO₃), potassium carbonate (K₂CO₃), triethylamine (TEA), or diisopropylethylamine (DIPEA), in an organic solvent such as dimethylformamide, acetonitrile, dichloromethane, or dimethyl sulfoxide. And, the reaction may be carried out at room temperature or under heating conditions (60 °C to 110 °C), but is not limited thereto.

In the Reaction Scheme 1, the reaction of Synthesis 2 may be carried out by reacting the compound of Formula II or III-a having the substituent M with Y-Q-L-halide or reacting the compound of Formula II or III-a having the substituent hal with Y-Q-L-B(OH)₂ or pinacolborane.

The reaction of Synthesis 2 may be carried out through the Suzuki reaction, using a palladium catalyst. The palladium catalyst includes palladium diacetate (Pd(OAc)₂), tetrakis(triphenylphosphine)palladium (Pd(PPh₃)₄), palladium di[1,1′-bis(diphenylphosphino)ferrocene]dichloride (PdCl₂(dppf)₂), or dichlorobis(triphenylphosphine)palladium(II) (PdCl₂(PPh₃)₂), etc. And, the reaction may be carried out in the presence of a base such as cesium carbonate (Cs₂CO₃) or potassium carbonate (K₂CO₃), in an organic solvent such as 1,4-dioxane, tetrahydrofuran, acetonitrile, 1,2-dimethoxyethane, or dimethylformamide and water, at 80 °C to 110 °C.

In addition, the reaction of Synthesis 2 may be also carried out through the Sonogashira reaction using a palladium and copper catalyst. The palladium and copper catalyst includes e.g., tetrakis(triphenylphosphine)palladium (Pd(PPh₃)₄), dichlorobis(triphenylphosphine)palladium(II) (PdCl₂(PPh₃)₂), and copper(I) iodide (CuI). And, the reaction may be carried out in the presence of a base such as triethylamine (TEA) or diisopropylethylamine (DIPEA), in an organic solvent such as dimethylformamide or tetrahydrofuran, at 60 °C to 110 °C.

In the Reaction Scheme 1, the reaction of Synthesis 3 may be carried out by reacting the compound of Formula IV with the compound of Formula V, using a palladium catalyst, through the Buchwald-hartwig reaction. The palladium catalyst includes palladium diacetate (Pd(OAc)₂), tris(dibenzylideneacetone)dipalladium (Pd₂(dba)₃), tetrakis(triphenylphosphine)palladium (Pd(PPh₃)₄), or palladium di[1,1′-bis(diphenylphosphino)ferrocene]dichloride (PdCl₂(dppf)₂), etc. The reaction may be carried out using a ligand and a base, in addition to the palladium catalyst. The ligand includes 2-dicyclohexylphosphino-2′,4′,6′-triisopropylbipheny 1 (XPhos), 4,5-bis(diphenylphosphino)-9,9-dimethylxanthene (Xantphos), (S)-2,2-bis(diphenylphosphino)-1,1-binaphthyl (BINAP), or 1,1′-bis(diphenylphosphino)ferr ocene (dppf), etc. The base includes cesium carbonate (Cs₂CO₃), potassium carbonate (K₂CO₃), potassium fluoride (KF), potassium phosphate (K₃PO₄), sodium tert-butoxide (NaOtBu), etc. The reaction may be carried out in an organic solvent such as benzene, toluene, 1,4-dioxane, tetrahydrofuran, acetonitrile, 1,2-dimethoxyethane, or dimethylformamide, at 60 °C to 110 °C.

The compound of Formula V in the Reaction Scheme 1 may be prepared from e.g., a compound of Formula Va, according to the following Reaction Scheme 2.

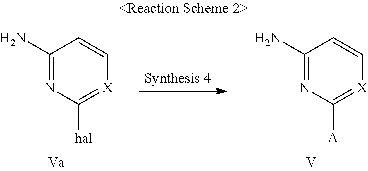

In the Reaction Scheme 2, hal is halogen and X and A are the same as defined in the above.

In the Reaction Scheme 2, the reaction of Synthesis 4 may be carried out by reacting the compound of Formula Va with A-boronic acid or A-pinacolborane through the Suzuki reaction, using a palladium catalyst. The palladium catalyst includes palladium diacetate (Pd(OAc)₂), tetrakis(triphenylphosphine)palladium (Pd(PPh₃)₄), palladium di[1,1′-bis(diphenylphosphino)ferrocene]dichloride (PdCl₂(dppf)₂), or dichlorobis(triphenylphosphine)palladium(II) (PdCl₂(PPh₃)₂), etc. And, the reaction may be carried out in the presence of a base such as cesium carbonate (Cs₂CO₃) or potassium carbonate (K₂CO₃), in an organic solvent such as 1,4-dioxane, tetrahydrofuran, acetonitrile, 1,2-dimethoxyethane, or dimethylformamide and water, at 80 °C to 110 °C.

The pyrimidine/pyridine derivative or pharmaceutically acceptable salt thereof according to the present invention, i.e., the compound of Formula 1 or pharmaceutically acceptable salt thereof has an excellent inhibitory activity against hematopoietic progenitor kinase 1 (HPK1) and thus can be usefully applied for the prevention or treatment of various diseases mediated by HPK1.

Therefore, the present invention includes, within its scope, a pharmaceutical composition for inhibiting hematopoietic progenitor kinase 1 comprising a therapeutically effective amount of the compound of Formula 1 or pharmaceutically acceptable salt thereof as an active ingredient. In an embodiment, the present invention provides a pharmaceutical composition for preventing or treating the diseases associated with HPK1 such as a cancer including a blood cancer or a solid tumor, for example melanoma, blastoma, sarcoma, lymphoma, leukemia, bladder cancer, brain tumor, breast cancer, cervical cancer, colorectal cancer, esophageal cancer, endometrioma, hepatic cancer, laryngeal cancer, lung cancer, osteosarcoma, ovarian cancer, pancreatic cancer, prostate cancer, renal cancer, thyroid cancer, ependymoma, glioblastoma, bullous cell tumor, neuroblastoma, osteosarcoma, rhabdomyosarcoma, or nephroblastoma, comprising a therapeutically effective amount of the compound of Formula 1 or pharmaceutically acceptable salt thereof as an active ingredient.

The pharmaceutical composition of the present invention may comprise a pharmaceutically acceptable carrier, such as diluents, disintegrants, sweeteners, lubricants, or flavoring agents, which is conventionally used in the art. The pharmaceutical composition may be formulated to an oral dosage form such as tablets, capsules, powders, granules, suspensions, emulsions, or syrups; or a parenteral dosage form such as solutions for external use, suspensions for external use, emulsions for external use, gels (e.g., ointment), inhalations, nebulizations, injections, according to conventional methods. The dosage form may be formulated to various forms, e.g., dosage forms for single administration or for multiple administrations.

The pharmaceutical composition of the present invention may comprise, for example, a diluent (e.g., lactose, corn starch, etc.; a lubricant (e.g., magnesium stearate); an emulsifying agent; a suspending agent; a stabilizer; and/or an isotonic agent. If necessary, the composition further comprises sweeteners and/or flavoring agents.

The composition of the present invention may be administered orally or parenterally, including inhalant, intravenous, intraperitoneal, subcutaneous, rectal and topical routes of administration. Therefore, the composition of the present invention may be formulated into various forms such as tablets, capsules, aqueous solutions or suspensions. In the case of tablets for oral administration, carriers such as lactose, corn starch, and lubricating agents, e.g. magnesium stearate, are conventionally used. In the case of capsules for oral administration, lactose and/or dried corn starch can be used as a diluent. When an aqueous suspension is required for oral administration, the active ingredient may be combined with emulsifying and/or suspending agents. If desired, certain sweetening and/or flavoring agents may be used. For intramuscular, intraperitoneal, subcutaneous and intravenous administration, sterile solutions of the active ingredient are usually prepared, and the pH of the solutions should be suitably adjusted and buffered. For intravenous administration, the total concentration of solutes should be controlled in order to render the preparation isotonic. The composition of the present invention may be in the form of an aqueous solution containing pharmaceutically acceptable carriers, e.g., saline having a pH level of 7.4. The solutions may be introduced into a patient's intramuscular bloodstream by local bolus injection.

The compound of Formula 1 or pharmaceutically acceptable salt thereof may be singly or multiply administered in an effective amount ranging from about 1 mg/kg per day to about 1000 mg/kg per day, preferably from about 100 mg/kg per day to about 800 mg/kg per day, to a subject patient. Of course, the dosage may be changed according to the patient's age, weight, susceptibility, symptom, or activity of the compound.

The present invention includes, within its scope, a method for inhibiting hematopoietic progenitor kinase 1 in a mammal, comprising administering a therapeutically effective amount of the compound of Formula 1 or pharmaceutically acceptable salt thereof to the mammal in need thereof. In an embodiment, the present invention provides a method for treating the diseases associated with HPK1 such as a cancer including a blood cancer or a solid tumor, for example melanoma, blastoma, sarcoma, lymphoma, leukemia, bladder cancer, brain tumor, breast cancer, cervical cancer, colorectal cancer, esophageal cancer, endometrioma, hepatic cancer, laryngeal cancer, lung cancer, osteosarcoma, ovarian cancer, pancreatic cancer, prostate cancer, renal cancer, thyroid cancer, ependymoma, glioblastoma, bullous cell tumor, neuroblastoma, osteosarcoma, rhabdomyosarcoma, or nephroblastoma, comprising administering a therapeutically effective amount of the compound of Formula 1 or pharmaceutically acceptable salt thereof to the mammal in need thereof.

The present invention also provides a use of the compound of Formula 1 or pharmaceutically acceptable salt thereof for the manufacture of a medicament for inhibiting hematopoietic progenitor kinase 1 in a mammal. In an embodiment, the present invention provides a use of the compound of Formula 1 or pharmaceutically acceptable salt thereof for the manufacture of a medicament for preventing or treating the diseases associated with HPK1 such as a cancer including a blood cancer or a solid tumor, for example melanoma, blastoma, sarcoma, lymphoma, leukemia, bladder cancer, brain tumor, breast cancer, cervical cancer, colorectal cancer, esophageal cancer, endometrioma, hepatic cancer, laryngeal cancer, lung cancer, osteosarcoma, ovarian cancer, pancreatic cancer, prostate cancer, renal cancer, thyroid cancer, ependymoma, glioblastoma, bullous cell tumor, neuroblastoma, osteosarcoma, rhabdomyosarcoma, or nephroblastoma.

Hereinafter, the present invention will be described in more detail through examples and test examples. However, these examples and test examples are provided for illustration purposes only and are not intended to limit the scope of the invention.

The analyses of the compounds prepared below were carried out as follows: Nuclear magnetic resonance (NMR) spectrum analysis was carried out using Bruker 400 MHz spectrometer and the chemical shifts thereof were analyzed in ppm. And, the indicated molecular weights were measured by using liquid chromatograph/mass selective detector (MSD) of Agilent 1260 Infinity series equipped with an electrostatic spray interface, which depicts the m/z ratio values in ESI+ (electrospray ionization (cation)) by using Single Quadrupole, with the [M+H]+ peak represented. The purification of the compounds synthesized below was carried out with medium pressure liquid chromatography (MPLC) using the CombiFlash NextGen 300+ (Teledyne ISCO, USA). In addition, the starting materials for each example were known compounds that were synthesized according to literatures or purchased from the commercial markets.

The following Preparations show the methods for synthesizing the intermediates for preparing the compounds or salts thereof of the present invention (i.e., the compounds of Examples).

### Preparation A1-1: (S)-(6-((2-chloropyrimidin-4-yl)amino)-4-(3-hydroxypiperidin-1-yl)pyridin-3-yl)(cyclopropyl)methanone

### Step 1: (S)-(6-chloro-4-(3-hydroxypiperidin-1-yl)pyridin-3-yl)(cyclopropyl)methanone

Cyclopropyl(4,6-dichloropyridin-3-yl)methanone (3.0 g, 13.89 mmol) was dissolved in anhydrous acetonitrile (70.0 mL) and then (S)-3-hydroxypiperidine hydrochloride (2.1 g, 15.27 mmol) and diisopropylethylamine (3.8 mL, 27.77 mmol) were added dropwise thereto. The reaction mixture was stirred at room temperature overnight. Water was added to the reaction mixture and then the aqueous layer was extracted with ethyl acetate. The extracted organic layer was dried over anhydrous magnesium sulfate and then filtered. The filtrate was concentrated and then purified by silica gel column chromatography to prepare the titled compound (2.24 g). (Yield: 57.4%) 1H NMR (CDCl₃, 400 MHz) δ 8.34 (s, 1H), 6.85 (s, 1H), 3.93-3.38 (m, 1H), 3.36-3.35 (m, 1H), 3.19-3.17 (m, 1H), 3.12-3.05 (m, 2H), 2.54-2.51 (m, 1H), 1.96-1.92 (m, 2H), 1.65-1.62 (m, 2H), 1.31-1.30 (m, 2H), 1.29-1.28 (m, 2H)

### Step 2: (S)-(6-((2-chloropyrimidin-4-yl)amino)-4-(3-hydroxypiperidin-1-yl)pyridin-3-yl)(cyclopropyl)methanone (A1-1)

(S)-(6-Chloro-4-(3-hydroxypiperidin-1-yl)pyridin-3-yl)(cyclopropyl)methanone (1.0 g, 23.56 mmol) was dissolved in anhydrous 1,4-dioxane (24.0 mL) and then 4-amino-2-chloropyrimidine (507.6 mg, 3.92 mmol), tris(dibenzylideneacetone)dipalladium (326.2 mg, 3.92 mmol), and 4 5-bis(diphenylphosphino)-9 9-dimethylxanthene (412.2 mg, 0.71 mmol) were added dropwise thereto. Then, 1.0 M lithium bis(trimethylsilyl)amide (7.1 mL, 7.12 mmol) dissolved in tetrahydrofuran was added to the mixed solution, which was then refluxed under stirring at 110 °C for 2 hours. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (400.0 mg). (Yield: 30.0%) 1H NMR (CDCl₃, 400 MHz) δ 8.39 (s, 1H), 8.32 (d, 1H), 7.45 (d, 1H), 7.3 (s, 1H), 3.97 (s, 1H), 3.44-3.43 (m, 1H), 3.27-3.24 (m, 1H), 3.23-3.10 (m, 2H), 2.63-2.60 (m, 1H), 2.59-2.41 (m, 1H), 2.01-1.91 (m, 2H), 1.70-1.58 (m, 2H), 1.32-1.25 (m, 2H), 1.07-1.04 (m, 2H)

### Preparation A1-2: (3S)-1-[2-[(2-chloropyrimidin-4-yl)amino]-5-(1-tetrahydropyran-4-ylpyrazol-4-yl)-4-pyridyl]piperidin-3-ol

(S)-1-(2-Chloro-5-(1-(tetrahydro-2H-pyran-4-yl)-1H-pyrazol-4-yl)pyridin-4-yl)piperidin-3-ol (A2-1) was dissolved in anhydrous 1,4-dioxane (27.0 mL) and then 4-amino-2-chloropyrimidine (1.07 g, 8.27 mmol), tris(dibenzylideneacetone)dipalladium (504.7 mg, 0.55 mmol), 2-dicyclohexylphosphino-2′,6′-diisopropoxybiphenyl (514.4 mg, 1.10 mmol), and sodium tert-butoxide (794.5 mg, 8.27 mmol) were added thereto. The reaction mixture was refluxed under stirring at 110 °C overnight. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (317.0 mg). (Yield: 25.2%) 1H NMR (CDCl₃, 400 MHz) δ 8.26 (s, 1H), 8.16 (s, 1H), 8.06 (d, 1H), 7.83 (s, 1H), 6.99 (s, 1H), 6.06 (d, 1H), 4.46-4.44 (m, 1H), 4.21-4.13 (m, 3H), 3.62-3.54 (m, 3H), 3.11-3.05 (m, 1H), 2.94-2.84 (m, 2H), 2.52-2.34 (m, 1H), 2.28-2.01 (m, 4H), 2.05-1.86 (m, 2H), 1.84-1.16 (m, 2H)

### Preparation A2-1: (S)-1-(2-chloro-5-(1-(tetrahydro-2H-pyran-4-yl)-1H-pyrazol-4-yl)pyridin-4-yl)piperidin-3-ol

### Step 1: (S)-1-(2-chloro-5-iodopyridin-4-yl)piperidin-3-ol (A2)

2-Chloro-4-fluoro-5-iodopyridine (1.0 g, 3.88 mmol) was dissolved in anhydrous dimethylformamide (20.0 mL) and then (S)-3-hydroxypiperidine hydrochloride (588.0 mg, 4.27 mmol) and diisopropylethylamine (1.6 mL, 11.65 mmol) were added dropwise thereto. The reaction mixture was stirred at 50 °C for 4 hours. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (800 mg). (Yield: 60.8%) 1H NMR (CDCl₃, 400 MHz) δ 8.57 (s, 1H), 6.87 (s, 1H), 4.04-3.99 (m, 1H), 3.34-3.31 (m, 1H), 3.07-3.04 (m, 2H), 3.01-2.96 (m, 1H), 2.24 (d, 1H), 2.06-1.99 (m, 1H), 1.92-1.86 (m, 1H), 1.80-1.73 (m, 2H)

### Step 2: (S)-1-(2-chloro-5-(1-(tetrahydro-2H-pyran-4-yl)-1H-pyrazol-4-yl)pyridin-4-yl)piperidin-3-ol (A2-1)

(S)-1-(2-Chloro-5-iodopyridin-4-yl)piperidin-3-ol (2.1 g, 6.29 mmol) was dissolved in anhydrous 1,4-dioxane (31.4 mL) and then 1-(tetrahydro-2H-pyran-4-yl)-1H-pyrazol-4-boronic acid pinacol ester (1.75 g, 6.29 mmol) and [1,1′-bis(diphenylphosphino)ferrocene]dichloropalladium(II)-dichloromethane complex (257.0 mg, 0.31 mmol), 2.0M sodium carbonate solution (10.0 mL, 20.0 mmol) were added dropwise thereto. Then, the mixed solution was stirred at 80 °C for 6 hours. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (1.61 g). (Yield: 70.3%) 1H NMR (CDCl₃, 400 MHz) δ 8.14 (s, 1H), 7.80 (d, 2H), 6.88 (s, 1H), 4.44-4.37 (m, 1H), 4.16-4.13 (m, 2H), 3.87-3.82 (m, 1H), 3.61-3.54 (m, 2H), 3.26-3.23 (m, 1H), 3.01-2.98 (m, 1H), 2.78-2.69 (m, 2H), 2.18-2.05 (m, 4H), 1.94-1.91 (m, 1H), 1.84-1.80 (m, 1H), 1.66-1.49 (m, 3H)

### Preparation A2-2: (S)-1-(2-chloro-5-(1-methyl-1H-pyrazol-4-yl)pyridin-4-yl)piperidin-3-ol

The titled compound was prepared in accordance with the same procedures as in Preparation A2-1, changing the boronic acid reagent to 1-methylpyrazol-4-boronic acid pinacol ester. 1H NMR (CDCl₃, 400 MHz) δ 8.11 (s, 1H), 7.78 (s, 1H), 7.67 (s, 1H), 6.87 (s, 1H), 3.97 (s, 3H), 3.86-3.72 (m, 1H), 3.27-3.25 (m, 1H), 3.03-3.00 (m, 1H), 2.78-2.66 (m, 2H), 1.95-1.92 (m, 1H), 1.82-1.80 (m, 1H), 1.79-1.65 (m, 1H), 1.50-1.48 (m, 1H)

### Preparation A2-3: (S)-1-(2-chloro-5-(1-isopropyl-1H-pyrazol-4-yl)pyridin-4-yl)piperidin-3-ol

The titled compound was prepared in accordance with the same procedures as in Preparation A2-1, changing the boronic acid reagent to 1-isopropylpyrazol-4-boronic acid pinacol ester. 1H NMR (CDCl₃, 400 MHz) δ 8.12 (s, 1H), 7.76 (s, 2H), 6.86 (s, 1H), 4.58 (septet, 1H), 3.92-3.85 (m, 1H), 3.27-3.24 (m, 1H), 3.01-2.98 (m, 1H), 2.76-2.66 (m, 2H), 1.94-1.91 (m, 1H), 1.87-1.82 (m, 1H), 1.81-1.79 (m, 1H), 1.68 (d, 6H), 1.52-1.50 (m, 1H)

### Preparation A2-4: (3S)-1-(2-chloro-5-(1-(tetrahydrofuran-3-yl)-1H-pyrazol-4-yl)pyridin-4-yl)piperidin-3-ol

The titled compound was prepared in accordance with the same procedures as in Preparation A2-1, changing the boronic acid reagent to 1-(oxolan-3-yl)-1H-pyrazol-4-boronic acid pinacol ester. 1H NMR (CDCl₃, 400 MHz) δ 8.13 (s, 1H), 7.84 (s, 1H), 7.78 (s, 1H), 6.87 (s, 1H), 5.05-5.04 (m, 1H), 4.18-4.12 (m, 3H), 4.07-4.04 (m, 1H), 3.99-3.93 (m, 1H), 3.84-3.72 (m, 1H), 3.24-3.21 (m, 1H), 3.00-2.98 (m, 1H), 2.78-2.69 (m, 2H), 2.55-2.48 (m, 1H), 2.40-2.36 (m, 1H), 1.93-1.90 (m, 1H), 1.81-1.72 (m, 1H), 1.69-1.67 (m, 1H), 1.51-1.49 (m, 1H)

### Preparation A2-5: (S)-1-(2-chloro-5-(1-(1-methylpiperidin-4-yl)-1H-pyrazol-4-yl)pyridin-4-yl)piperidin-3-ol

The titled compound was prepared in accordance with the same procedures as in Preparation A2-1, changing the boronic acid reagent to 1-methyl-4-(4-(4,4,5,5-tetramethyl-1,3,2-dioxaborolan-2-yl)-1H-pyrazol-1-yl)piperidine. 1H NMR (CDCl₃, 400 MHz) δ 8.15 (s, 1H), 7.88 (d, 2H), 6.91 (s, 1H), 4.82-4.76 (m, 2H), 3.88-3.85 (m, 1H), 3.28-3.24 (m, 1H), 3.03-2.99 (m, 1H), 2.78-2.69 (m, 2H), 1.98-1.94 (m, 1H), 1.84-1.80 (m, 1H), 1.64-1.62 (m, 1H), 1.56-1.49 (m, 2H)

### Preparation A2-6: (S)-1-(2-chloro-5-(1-(2,2,2-trifluoroethyl)-1H-pyrazol-4-yl)pyridin-4-yl)piperidin-3-ol

The titled compound was prepared in accordance with the same procedures as in Preparation A2-1, changing the boronic acid reagent to 4-(4,4,5,5-tetramethyl-1,3,2-dioxaborolan-2-yl)-1-(2,2,2-trifluoroethyl)-1H-pyrazole. 1H NMR (CDCl₃, 400 MHz) δ 8.13 (s, 1H), 7.96 (s, 2H), 6.89 (s, 1H), 4.80-4.74 (q, 2H), 3.85-3.84 (m, 1H), 3.26-3.23 (m, 1H), 3.01-2.98 (m, 1H), 2.76-2.67 (m, 2H), 1.95-1.92 (m, 1H), 181-1.79 (m, 1H), 1.62-1.60 (m, 1H), 1.50-1.47 (m, 1H)

### Preparation A2-7: (S)-1-(2-chloro-5-(1-(cyclopropylmethyl)-1H-pyrazol-4-yl)pyridin-4-yl)piperidin-3-ol

The titled compound was prepared in accordance with the same procedures as in Preparation A2-1, changing the boronic acid reagent to 1-(cyclopropylmethyl)-4-(4,4,5,5-tetramethyl-1,3,2-dioxaborolan-2-yl)-1H-pyrazole. 1H NMR (CDCl₃, 400 MHz) δ 8.14 (s, 1H), 7.85 (s, 1H), 7.77 (s, 1H), 6.87 (s, 1H), 4.05 (d, 2H), 3.92-3.86 (m, 1H), 3.28-3.25 (m, 1H), 3.03-3.00 (m, 1H), 2.78-2.67 (m, 2H), 1.94-1.91 (m, 1H), 1.82-1.80 (m, 1H), 1.75-1.71 (m, 1H), 1.55-1.54 (m, 2H), 1.52-1.50 (m, 1H), 0.92-0.71 (m, 2H), 0.69-0.68 (m, 2H)

### Preparation A2-8: (S)-1-(2-chloro-5-(1-(difluoromethyl)-1H-pyrazol-4-yl)pyridin-4-yl)piperidin-3-ol

The titled compound was prepared in accordance with the same procedures as in Preparation A2-1, changing the boronic acid reagent to 1-(difluoromethyl)pyrazol-4-boronic acid pinacol ester. 1H NMR (CDCl₃, 400 MHz) δ 8.18 (d, 2H), 7.99 (s, 1H), 7.26 (t, 1H), 6.93 (s, 1H), 3.91-3.86 (m, 1H), 3.27-3.23 (m, 1H), 3.04-2.99 (m, 1H), 3.80 (s, 2H), 2.07-1.94 (m, 1H), 1.88-1.82 (m, 1H), 1.63-1.62 (m, 1H), 1.55-1.50 (m, 2H)

### Preparation A2-9: (S)-1-(2-chloro-5-(1-(2-methoxyethyl)-1H-pyrazol-4-yl)pyridin-4-yl)piperidin-3-ol

The titled compound was prepared in accordance with the same procedures as in Preparation A2-1, changing the boronic acid reagent to 1-(2-methoxyethyl)-4-(4,4,5,5-tetramethyl-1,3,2-dioxaborolan-2-yl)-1H-pyrazole. 1H NMR (CDCl₃, 400 MHz) δ 8.16 (s, 1H), 7.91 (s, 1H), 7.78 (s, 1H), 6.88 (s, 1H), 4.37-4.31 (m, 2H), 3.87-3.78 (m, 3H), 3.39 (s, 3H), 3.14 (d, 1H), 3.04-2.99 (m, 1H), 2.86-2.81 (m, 1H), 2.78-2.73 (m, 1H), 1.90-1.88 (m, 2H), 1.71-1.68 (m, 1H), 1.58-1.53 (m, 1H)

### Preparation A2-10: (S)-1-(2-chloro-5-(1-(2,2-difluoroethyl)-1H-pyrazol-4-yl)pyridin-4-yl)piperidin-3-ol

The titled compound was prepared in accordance with the same procedures as in Preparation A2-1, changing the boronic acid reagent to 1-(2,2-difluoroethyl)-4-(4,4,5,5-tetramethyl-1,3,2-dioxaborolan-2-yl)-1H-pyrazole. 1H NMR (CDCl₃, 400 MHz) δ 8.15 (s, 1H), 7.86-7.85 (m, 2H), 6.91 (s, 1H), 6.32-6.02 (m, 1H), 4.58-4.50 (m, 2H), 3.90-3.84 (m, 1H), 3.27-3.23 (m, 1H), 3.04-3.00 (m, 1H), 2.79-2.67 (m, 2H), 1.98-1.94 (m, 1H), 1.86-1.80 (m, 1H), 1.57-1.48 (m, 3H)

### Preparation A2-11: (S)-1-(2-chloro-5-(1-(trifluoromethyl)-1H-pyrazol-4-yl)pyridin-4-yl)piperidin-3-ol

The titled compound was prepared in accordance with the same procedures as in Preparation A2-1, changing the boronic acid reagent to 4-(4,4,5,5-tetramethyl-1,3,2-dioxaborolan-2-yl)-1-(trifluoromethyl)-1H-pyrazole. 1H NMR (CDCl₃, 400 MHz) δ 8.14 (s, 1H), 7.83 (s, 1H), 7.76 (s, 1H), 6.85 (s, 1H), 4.58-4.52 (m, 1H), 4.37-4.35 (m, 2H), 3.81-3.79 (m, 3H), 3.40-3.35 (m, 4H), 3.05-3.02 (m, 1H), 2.61-2.56 (m, 1H), 1.93-1.92 (m, 1H), 1.65-1.63 (m, 1H), 1.46 (s, 9H), 1.41-1.31 (m, 1H)

### Preparation A2-12: (S)-1-(2-chloro-5-(4-(tetrahydro-2H-pyran-4-yl)phenyl)pyridin-4-yl)piperidin-3-ol

The titled compound was prepared in accordance with the same procedures as in Preparation A2-1, changing the boronic acid reagent to 4-(4-tetrahydropyranyl)phen ylboronic acid pinacol ester. 1H NMR (CDCl₃, 400 MHz) δ 8.03 (s, 1H), 7.44-7.42 (m, 2H), 7.32-7.30 (m, 2H), 6.86 (s, 1H), 4.12-4.10 (m, 2H), 3.71-3.70 (m, 1H), 3.59-3.53 (m, 2H), 3.13-3.11 (m, 1H), 2.92-2.90 (m, 1H), 2.84-2.70 (m, 3H), 1.87-1.82 (m, 5H), 1.79-1.68 (m, 1H), 1.56-1.44 (m, 3H)

### Preparation A2-13: (S)-1-(2-chloro-5-(4-(4-methylpiperazin-1-yl)phenyl)pyridin-4-yl)piperidin-3-ol

The titled compound was prepared in accordance with the same procedures as in Preparation A2-1, changing the boronic acid reagent to 4-(4-methyl-1-piperazinyl)p henylboronic acid pinacol ester. 1H NMR (CDCl₃, 400 MHz) δ 8.01 (s, 1H), 7.39 (d, 2H), 7.00 (d, 2H), 6.84 (s, 1H), 3.82-3.74 (m, 1H), 3.30-3.27 (m, 4H), 3.16-3.13 (m, 1H), 2.94-2.92 (m, 1H), 2.79-2.71 (m, 2H), 2.63-2.61 (m, 4H), 2.38 (s, 3H), 1.74-1.70 (m, 2H), 1.48-1.46 (m, 2H)

### Preparation A2-14: (S)-1-(6-chloro-6′-morpholino-[3,3′-bipyridin]-4-yl)piperidin-3-ol

The titled compound was prepared in accordance with the same procedures as in Preparation A2-1, changing the boronic acid reagent to 6-(morpholin-4-yl)pyridin-3-boronic acid pinacol ester. 1H NMR (CDCl₃, 400 MHz) δ 8.31 (s, 1H), 8.00 (s, 1H), 7.71 (d, 1H), 6.87 (s, 1H), 6.72 (d, 1H), 3.86-3.84 (m, 4H), 3.78-3.77 (m, 1H), 3.58-3.55 (m, 4H), 3.23-3.21 (m, 1H), 3.01-2.99 (m, 1H), 2.80-2.69 (m, 2H), 1.92-1.87 (m, 1H), 1.82-1.72 (m, 1H), 1.49-1.45 (m, 2H)

### Preparation A2-15: (S)-1-(6-chloro-[3,3′-bipyridin]-4-yl)piperidin-3-ol

The titled compound was prepared in accordance with the same procedures as in Preparation A2-1, changing the boronic acid reagent to 3-pyridylboronic acid. 1H NMR (CDCl₃, 400 MHz) δ 8.81 (s, 1H), 8.70-8.68 (m, 1H), 8.06 (s, 1H), 7.88-7.87 (m, 1H), 7.45-7.42 (m, 1H), 6.93 (s, 1H), 3.82-3.75 (m, 1H), 3.21-3.18 (m, 1H), 2.75-2.73 (m, 1H), 2.72-2.70 (m, 2H), 1.72-1.70 (m, 2H), 1.48-1.47 (m, 1H)

### Preparation A2-16: (S)-1-(6-chloro-6′-ethoxy-[3,3′-bipyridin]-4-yl)piperidin-3-ol

The titled compound was prepared in accordance with the same procedures as in Preparation A2-1, changing the boronic acid reagent to 6-ethoxypyridin-3-boronic acid. 1H NMR (CDCl₃, 400 MHz) δ 8.27 (s, 1H), 8.03 (s, 1H), 7.77 (d, 1H), 6.89 (s, 1H), 6.83 (d, 1H), 4.41 (q, 2H), 3.77-3.75 (m, 1H), 3.24-3.22 (m, 1H), 2.98-2.97 (m, 1H), 2.76-2.68 (m, 2H), 1.90-1.87 (m, 1H), 1.74-1.72 (m, 2H), 1.68 (t, 3H)

### Preparation A2-17: (S)-1-(6-chloro-6′-(trifluoromethyl)-[3,3′-bipyridin]-4-yl)piperidin-3-ol

The titled compound was prepared in accordance with the same procedures as in Preparation A2-1, changing the boronic acid reagent to 2-(trifluoromethyl)pyridin-5-boronic acid. 1H NMR (CDCl₃, 400 MHz) δ 8.93 (s, 1H), 8.07 (s, 1H), 8.05 (d, 1H), 7.82 (d, 1H), 6.97 (s, 1H), 3.78-3.76 (m, 1H), 3.23-3.22 (m, 1H), 3.20-3.19 (m, 1H), 2.96-2.73 (m, 2H), 1.95-1.92 (m, 1H), 1.91-1.88 (m, 1H), 1.69-1.49 (m, 2H)

### Preparation A2-18: (S)-1-(2-chloro-5-(imidazo[1,2-a]pyridin-6-yl)pyridin-4-yl)piperidin-3-ol

The titled compound was prepared in accordance with the same procedures as in Preparation A2-1, changing the boronic acid reagent to imidazo[1,2-a]pyridin-6-ylboronic acid. 1H NMR (CDCl₃, 400 MHz) δ 8.28 (s, 1H), 8.08 (s, 1H), 7.62-7.59 (m, 1H), 7.52-7.51 (m, 1H), 7.40-7.38 (m, 2H), 6.92 (s, 1H), 3.82-3.78 (m, 1H), 3.34-3.20 (m, 1H), 3.05-2.92 (m, 1H), 2.81-2.73 (m, 2H), 2.12-2.05 (m, 1H), 1.91-1.84 (m, 1H), 1.49-1.47 (m, 2H)

### Preparation A2-19: (S)-1-(5-([1,2,4]triazolo[4,3-a]pyridin-6-yl)-2-chloropyridin-4-yl)piperidin-3-ol

The titled compound was prepared in accordance with the same procedures as in Preparation A2-1, changing the boronic acid reagent to [1,2,4]triazolo[4,3-a]pyrid in-6-ylboronic acid. 1H NMR (CDCl₃, 400 MHz) δ 8.80 (s, 1H), 8.30 (s, 1H), 8.08 (s, 1H), 7.79 (d, 1H), 7.54 (d, 1H), 6.95 (s, 1H), 3.92-3.81 (m, 1H), 3.33-3.30 (m, 1H), 3.12-3.10 (m, 1H), 2.90-2.88 (m, 1H), 2.79-2.74 (m, 1H), 1.96-1.93 (m, 1H), 1.82-1.73 (m, 1H), 1.51-1.48 (m, 2H)

### Preparation A2-20: (3S)-1-[2-chloro-5-[1-(2,2,6,6-tetramethyltetrahydropyran-4-yl) pyrazol-4-yl]-4-pyridyl]piperidin-3-ol

The titled compound was prepared in accordance with the same procedures as in Preparation A2-1, changing the boronic acid reagent to 4-(4,4,5,5-tetramethyl-1,3,2-dioxaborolan-2-yl)-1-(2,2,6,6-tetramethyltetrahydropyran-4-yl)pyrazole. 1H NMR (CDCl₃, 400 MHz) δ 8.14 (s, 1H), 7.82 (s, 1H), 7.77 (s, 1H), 6.90 (s, 1H), 4.80-4.70 (m, 1H), 3.95-3.85 (m, 1H), 3.70-3.65 (m, 1H), 3.30-3.20 (m, 1H), 3.10-3.00 (m, 1H), 2.80-2.65 (m, 2H), 2.20-2.10 (m, 2H), 2.00-1.90 (m, 1H), 1.90-1.80 (m, 2H), 1.70-1.60 (m, 1H), 1.60-1.50 (m, 1H), 1.42 (s, 6H), 1.34 (s, 6H)

### Preparation A3-1: tert-butyl (S)-(1-(2-chloro-5-(1-(tetrahydro-2H-pyran-4-yl)-1H-pyrazol-4-yl)pyridin-4-yl)piperidin-3-yl)carbamate

### Step 1: tert-butyl (S)-(1-(2-chloro-5-iodopyridin-4-yl)piperidin-3-yl)carbamate (A3)

2-Chloro-4-fluoro-5-iodopyridine (5.0 g, 19.42 mmol) was dissolved in anhydrous dimethylformamide (90.6 mL) and then (S)-3-(tert-butoxycarbonylamino)piperidine (4.28 g, 21.36 mmol) and diisopropylethylamine (7.9 mL, 58.26 mmol) were added dropwise thereto. The reaction mixture was stirred at 50 °C for 4 hours. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (5.9 g). (Yield: 69.9%) 1H NMR (CDCl₃, 400 MHz) δ 8.57 (s, 1H), 6.85 (s, 1H), 4.92 (brs, 1H), 3.95-3.94 (m, 1H), 3.34 (d, 1H), 3.08-3.04 (m, 2H), 2.90-2.85 (m, 1H), 2.06-1.94 (m, 3H), 1.46 (s, 9H)

### Step 2: tert-butyl (S)-(1-(2-chloro-5-(1-(tetrahydro-2H-pyran-4-yl)-1H-pyrazol-4-yl)pyridin-4-yl)piperidin-3-yl)carbamate (A3-1)

tert-Butyl (S)-(1-(2-chloro-5-iodopyridin-4-yl)piperidin-3-yl)carbamate (3.0 g, 6.85 mmol) was dissolved in anhydrous 1,4-dioxane (34.3 mL) and then 1-(tetrahydro-2H-pyran-4-yl)-1H-pyrazol-4-boronic acid pinacol ester (2.9 g, 10.28 mmol), [1,1′-bis(diphenylphosphino)ferrocene]dichloropalladium(II)-dichloromethane complex (559.7 mg, 0.68 mmol), and 2.0M sodium carbonate solution (12.1 mL, 24.2 mmol) were added dropwise thereto. Then, the mixed solution was stirred at 100 °C for 4 hours. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (2.3 g). (Yield: 73.0%) 1H NMR (CDCl₃, 400 MHz) δ 8.15 (s, 1H), 7.81 (s, 1H), 7.77 (s, 1H), 6.86 (s, 1H), 4.55-4.45 (m, 1H), 4.45-4.35 (m, 1H), 4.20-4.10 (m, 2H), 3.80-3.70 (m, 1H), 3.61 (t, 2H), 3.50-3.40 (m, 1H), 3.15-3.00 (m, 1H), 2.65-2.55 (m, 1H), 2.55-2.45 (m, 1H), 2.25-2.05 (m, 4H), 2.00-1.90 (m, 1H), 1.75-1.65 (m, 1H), 1.45 (s, 9H), 1.40-1.30 (m, 1H)

### Preparation A3-2: tert-butyl (S)-(1-(2-chloro-5-(1-(difluoromethyl)-1H-pyrazol-4-yl)pyridin-4-yl)piperidin-3-yl)carbamate

The titled compound was prepared in accordance with the same procedures as in Preparation A3-1, changing the boronic acid reagent to 1-(difluoromethyl)pyrazol-4-boronic acid pinacol ester. 1H NMR (CDCl₃, 400 MHz) δ 8.13 (d, 2H), 7.94 (s, 1H), 7.26 (t, 1H), 6.90 (s, 1H), 4.48-4.47 (m, 1H), 3.76-3.67 (m, 1H), 3.46-3.43 (m, 1H), 3.08-3.05 (m, 1H), 2.62-2.52 (m, 2H), 1.98-1.96 (m, 1H), 1.73-1.64 (m, 1H), 1.45 (s, 9H), 1.38-1.31 (m, 2H)

### Preparation A3-3: tert-butyl (S)-(1-(2-chloro-5-(1-(1-methylpiperidin-4-yl)-1H-pyrazol-4-yl)pyridin-4-yl)piperidin-3-yl)carbamate

The titled compound was prepared in accordance with the same procedures as in Preparation A3-1, changing the boronic acid reagent to 1-methyl-4-(4-(4,4,5,5-tetramethyl-1,3,2-dioxaborolan-2-yl)-1H-pyrazol-1-yl)piperidine. 1H NMR (CDCl₃, 400 MHz) δ 8.14 (s, 1H), 7.79-7.75 (m, 2H), 6.85 (s, 1H), 4.57-4.48 (m, 1H), 4.23-4.16 (m, 1H), 3.76-3.70 (m, 1H), 3.42 (d, 1H), 3.07-3.03 (m, 3H), 2.58-2.55 (m, 2H), 2.36 (s, 3H), 2.23-2.12 (m, 7H), 1.96-1.94 (m, 1H), 1.65-1.54 (m, 2H), 1.43 (s, 9H)

### Preparation A3-4: tert-butyl (S)-(1-(2-chloro-5-(1-(2,2,2-trifluoroethyl)-1H-pyrazol-4-yl)pyridin-4-yl)piperidin-3-yl)carbamate

The titled compound was prepared in accordance with the same procedures as in Preparation A3-1, changing the boronic acid reagent to 4-(4,4,5,5-tetramethyl-1,3,2-dioxaborolan-2-yl)-1-(2,2,2-trifluoroethyl)pyrazole. 1H NMR (CDCl₃, 400 MHz) δ 8.15 (s, 1H), 7.88 (s, 1H), 7.82 (s, 1H), 6.88 (s, 1H), 4.83-4.78 (m, 2H), 4.50-4.42 (m, 1H), 3.73-3.69 (m, 1H), 3.44-3.41 (m, 1H), 3.11-3.09 (m, 1H), 2.63-2.58 (m, 1H), 2.45-2.43 (m, 1H), 2.00-1.94 (m, 1H), 1.73-1.70 (m, 1H), 1.44 (s, 9H), 1.35-1.28 (m, 1H)

### Preparation A3-5: tert-butyl (S)-(1-(2-chloro-5-(1-(2-methoxyethyl)-1H-pyrazol-4-yl)pyridin-4-yl)piperidin-3-yl)carbamate

The titled compound was prepared in accordance with the same procedures as in Preparation A3-1, changing the boronic acid reagent to 1-(2-methoxyethyl)-4-(4,4,5,5-tetramethyl-1,3,2-dioxaborolan-2-yl)pyrazole. 1H NMR (CDCl₃, 400 MHz) δ 8.14 (s, 1H), 7.83 (s, 1H), 7.76 (s, 1H), 6.85 (s, 1H), 4.58-4.52 (m, 1H), 4.37-4.35 (m, 2H), 3.81-3.79 (m, 3H), 3.40-3.35 (m, 4H), 3.05-3.02 (m, 1H), 2.61-2.56 (m, 1H), 1.93-1.92 (m, 1H), 1.65-1.63 (m, 1H), 1.46 (s, 9H), 1.41-1.31 (m, 1H)

### Preparation A3-6: tert-butyl (S)-(1-(2-chloro-5-(1-(3-methoxypropyl)-1H-pyrazol-4-yl)pyridin-4-yl)piperidin-3-yl)carbamate

The titled compound was prepared in accordance with the same procedures as in Preparation A3-1, changing the boronic acid reagent to 1-(3-methoxypropyl)pyrazol-4-boronic acid pinacol ester. 1H NMR (CD₃OD, 400 MHz) δ 8.07 (s, 1H), 8.00 (s, 1H), 7.81 (s, 1H), 7.02 (s, 1H), 4.30 (t, 2H), 3.59 (brs, 1H), 3.43-3.32 (m, 3H), 3.32 (s, 3H), 3.22 (d, 1H), 2.62 (t, 1H), 2.51 (t, 1H), 2.17-2.11 (m, 2H), 1.96-1.93 (m, 1H), 1.73 (brs, 1H), 1.70-1.62 (m, 1H), 1.59 (s, 9H), 1.51-1.43 (m, 1H)

### Preparation A3-7: tert-butyl (S)-(1-(2-chloro-5-(1-methyl-3-(trifluoromethyl)-1H-pyrazol-4-yl)pyridin-4-yl)piperidin-3-yl)carbamate

The titled compound was prepared in accordance with the same procedures as in Preparation A3-1, changing the boronic acid reagent to 1-methyl-3-(trifluoromethyl)pyrazol-4-boronic acid pinacol ester. 1H NMR (CD₃OD, 400 MHz) δ 7.91 (s, 1H), 7.88 (s, 1H), 7.03 (s, 1H), 4.09 (s, 1H), 3.34-3.32 (m, 2H), 3.27-3.23 (m, 1H), 2.71 (t, 1H), 2.38 (t, 1H), 1.90-1.86 (m, 1H), 1.71 (brs, 1H), 1.60-1.54 (m, 2H), 1.44 (s, 9H)

### Preparation A3-8: tert-butyl (S)-(1-(2-chloro-5-(1-(2-(dimethylamino)-2-oxoethyl)-1H-pyrazol-4-yl)pyridin-4-yl)piperidin-3-yl)carbamate

The titled compound was prepared in accordance with the same procedures as in Preparation A3-1, changing the boronic acid reagent to N,N-dimethyl-2-(4-(4,4,5,5-tetramethyl-1,3,2-dioxaborolan-2-yl)-1H-pyrazol-1-yl)acetamide. 1H NMR (CD₃OD, 400 MHz) δ 8.13 (s, 1H), 8.04 (s, 1H), 7.85 (s, 1H), 5.24-5.14 (m, 2H), 3.63-3.59 (m, 1H), 3.45-3.41 (m, 2H), 3.17 (s, 3H), 3.01 (s, 3H), 2.65-2.55 (m, 2H), 2.03-1.97 (m, 1H), 1.96-1.92 (m, 1H), 1.74-1.66 (m, 1H), 1.46 (s, 9H), 1.40 (brs, 1H)

### Preparation A3-9: tert-butyl (S)-(1-(2-chloro-5-(1-(2-(methoxymethoxy)ethyl)-1H-pyrazol-4-yl)pyridin-4-yl)piperidin-3-yl)carbamate

The titled compound was prepared in accordance with the same procedures as in Preparation A3-1, changing the boronic acid reagent to 1-(2-(methoxymethoxy)ethyl)-4-(4,4,5,5-tetramethyl-1,3,2-dioxaborolan-2-yl)-1H-pyrazole. 1H NMR (CDCl₃, 400 MHz) δ 8.09 (d, 2H), 7.85 (s, 1H), 7.04 (s, 1H), 4.60 (s, 2H), 4.43-4.42 (m, 2H), 3.94 (t, 2H), 3.63-3.61 (m, 1H), 3.43-3.41 (m, 1H), 3.26 (s, 3H), 3.25 (brs, 1H), 2.62-2.53 (m, 2H), 1.97-1.93 (m, 2H), 1.73-1.69 (m, 2H), 1.45 (s, 9H), 1.39-1.31 (m, 1H)

### Preparation A3-10: tert-butyl (S)-(1-(2-chloro-5-(1-(2,2-difluoroethyl)-1H-pyrazol-4-yl)pyridin-4-yl)piperidin-3-yl)carbamate

The titled compound was prepared in accordance with the same procedures as in Preparation A3-1, changing the boronic acid reagent to 1-(2,2-difluoroethyl)-4-(4,4,5,5-tetramethyl-1,3,2-dioxaborolan-2-yl)pyrazole. 1H NMR (CDCl₃, 400 MHz) δ 8.14 (s, 1H), 7.83 (s, 1H), 7.82 (s, 1H), 6.88 (s, 1H), 6.34-6.19 (m, 1H), 4.60-4.53 (m, 2H), 3.82-3.70 (m, 1H), 3.46-3.43 (m, 1H), 3.12-3.09 (m, 1H), 2.64-2.61 (m, 1H), 2.53-2.42 (m, 1H), 1.98-1.92 (m, 1H), 1.83-1.74 (m, 1H), 1.72-1.62 (m, 1H), 1.57 (s, 9H), 1.45-1.43 (m, 1H)

### Preparation A3-11: tert-butyl (S)-(1-(2-chloro-5-(1-(trifluoromethyl)-1H-pyrazol-4-yl)pyridin-4-yl)piperidin-3-yl)carbamate

The titled compound was prepared in accordance with the same procedures as in Preparation A3-1, changing the boronic acid reagent to 4-(4,4,5,5-tetramethyl-1,3,2-dioxaborolan-2-yl)-1-(trifluoromethyl)-1H-pyrazole. 1H NMR (CDCl₃, 400 MHz) δ 8.14 (s, 1H), 8.12 (s, 1H), 8.06 (s, 1H), 6.92 (s, 1H), 4.47 (brs, 1H), 3.81-3.71 (m, 1H), 3.49-3.47 (m, 1H), 3.09-3.06 (m, 1H), 2.65-2.53 (m, 2H), 1.99-1.96 (m, 1H), 1.73-1.68 (m, 1H), 1.46 (s, 9H), 1.45-1.37 (m, 2H)

### Preparation A3-12: tert-butyl (S)-(1-(2-chloro-5-(1-(2-morpholinoethyl)-1H-pyrazol-4-yl)pyridin-4-yl)piperidin-3-yl)carbamate

The titled compound was prepared in accordance with the same procedures as in Preparation A3-1, changing the boronic acid reagent to 4-[2-[4-(4,4,5,5-tetramethy 1-1,3,2-dioxaborolan-2-yl)pyrazol-1-yl]ethyl]morpholine. 1H NMR (CDCl₃, 400 MHz) δ 8.14 (s, 1H), 7.81 (s, 1H), 7.77 (s, 1H), 6.87 (s, 1H), 4.55-4.45 (m, 1H), 4.33 (t, 2H), 3.80-3.75 (m, 1H), 3.72 (t, 4H), 3.50-3.40 (m, 1H), 3.15-3.05 (m, 1H), 2.89 (t, 2H), 2.65-2.60 (m, 1H), 2.60-2.45 (m, 5H), 2.00-1.90 (m, 1H), 1.75-1.65 (m, 1H), 1.46 (s, 9H), 1.40-1.30 (m, 1H)

### Preparation A3-13: tert-butyl (S)-(1-(2-chloro-5-(1-(2-(pyrrolidin-1-yl)ethyl)-1H-pyrazol-4-yl)pyridin-4-yl)piperidin-3-yl)carbamate

The titled compound was prepared in accordance with the same procedures as in Preparation A3-1, changing the boronic acid reagent to 1-(2-pyrrolidin-1-ylethyl)-4-(4,4,5,5-tetramethyl-1,3,2-dioxaborolan-2-yl)pyrazole. 1H NMR (CDCl₃, 400 MHz) δ 8.12 (s, 1H), 7.80 (s, 1H), 7.74 (s, 1H), 6.84 (s, 1H), 4.60-4.50 (m, 1H), 4.30 (t, 2H), 3.80-3.70 (m, 1H), 3.45-3.35 (m, 1H), 3.10-2.90 (m, 3H), 2.70-2.50 (m, 6H), 2.00-1.90 (m, 1H), 1.85-1.70 (m, 4H), 1.70-1.60 (m, 1H), 1.44 (s, 9H), 1.40-1.30 (m, 1H)

### Preparation A3-14: tert-butyl N-[(3S)-1-[2-chloro-5-[1-(2-cyanoethyl)pyrazol-4-yl]-4-pyridyl]-3-piperidyl]carbamate

The titled compound was prepared in accordance with the same procedures as in Preparation A3-1, changing the boronic acid reagent to 3-[4-(4,4,5,5-tetramethyl-1,3,2-dioxaborolan-2-yl)pyrazol-1-yl]propanenitrile. 1H NMR (CDCl₃, 400 MHz) δ 8.15 (s, 1H), 7.86 (s, 1H), 7.83 (s, 1H), 6.89 (s, 1H), 4.50 (t, 2H), 4.50-4.40 (m, 1H), 3.75-3.65 (m, 1H), 3.50-3.40 (m, 1H), 3.20-3.05 (m, 2H), 3.09 (t, 2H), 2.70-2.60 (m, 1H), 2.45-2.35 (m, 1H), 2.05-1.95 (m, 1H), 1.80-1.70 (m, 1H), 1.45 (s, 9H), 1.40-1.30 (m, 1H)

### Preparation A3-15: tert-butyl (S)-3-(3-(4-(3-((tert-butoxycarbonyl)amino)piperidin-1-yl)-6-chloropyridin-3-yl)-1H-pyrazol-1-yl)azetidin-1-carboxylate

The titled compound was prepared in accordance with the same procedures as in Preparation A3-1, changing the boronic acid reagent to tert-butyl 3-(3-(4,4,5,5-tetramethyl-1,3,2-dioxaborolan-2-yl)-1H-pyrazol-1-yl)azetidin-1-carboxylate. 1H NMR (CDCl₃, 400 MHz) δ 8.31 (s, 1H), 7.65 (s, 1H), 6.86 (s, 1H), 6.60 (s, 1H), 5.25-5.15 (m, 1H), 5.75-5.65 (m, 1H), 4.43 (t, 2H), 4.40-4.20 (m, 2H), 3.75-3.65 (m, 1H), 3.30-3.20 (m, 1H), 3.10-3.00 (m, 1H), 2.80-2.70 (m, 1H), 2.65-2.55 (m, 1H), 1.90-1.80 (m, 1H), 1.80-1.65 (m, 1H), 1.47 (s, 9H), 1.44 (s, 9H), 1.40-1.30 (m, 1H)

### Preparation A3-16: tert-butyl (S)-(1-(6-chloro-6′-(dimethylamino)-[3,3′-bipyridin]-4-yl)piperidin-3-yl)carbamate

The titled compound was prepared in accordance with the same procedures as in Preparation A3-1, changing the boronic acid reagent to 6-dimethylaminopyridin-3-boronic acid pinacol ester. 1H NMR (CDCl₃, 400 MHz) δ 8.23 (d, 1H), 8.00 (s, 1H), 7.65 (d, 1H), 6.84 (s, 1H), 6.59 (d, 1H), 4.55-4.45 (m, 1H), 3.80-3.65 (m, 1H), 3.40-3.30 (m, 1H), 3.15 (s, 6H), 3.10-3.00 (m, 1H), 2.70-2.55 (m, 2H), 1.90-1.80 (m, 1H), 1.60-1.48 (m, 1H), 1.45 (s, 9H), 1.35-1.30 (m, 1H)

### Preparation A3-17: tert-butyl (S)-(1-(6-chloro-6′-(pyrrolidin-1-yl)-[3,3′-bipyridin]-4-yl)piperidin-3-yl)carbamate

The titled compound was prepared in accordance with the same procedures as in Preparation A3-1, changing the boronic acid reagent to 2-pyrrolidin-1-yl-5-(4,4,5,5-tetramethyl-1,3,2-dioxaborolan-2-yl)pyridine. 1H NMR (CDCl₃, 400 MHz) δ 8.22 (d, 1H), 8.00 (s, 1H), 7.64 (d, 1H), 6.84 (s, 1H), 6.44 (d, 1H), 4.55-4.45 (m, 1H), 3.80-3.65 (m, 1H), 3.51 (t, 4H), 3.40-3.30 (m, 1H), 3.10-3.00 (m, 1H), 2.70-2.55 (m, 2H), 2.05 (t, 4H), 1.90-1.80 (m, 1H), 1.60-1.48 (m, 1H), 1.45 (s, 9H), 1.35-1.30 (m, 1H)

### Preparation A3-18: tert-butyl (S)-(1-(6-chloro-6′-(piperidin-1-yl)-[3,3′-bipyridin]-4-yl)piperidin-3-yl)carbamate

The titled compound was prepared in accordance with the same procedures as in Preparation A3-1, changing the boronic acid reagent to 6-(piperidin-1-yl)pyridin-3-boronic acid pinacol ester. 1H NMR (CDCl₃, 400 MHz) δ 8.23 (d, 1H), 8.00 (s, 1H), 7.65 (d, 1H), 6.85 (s, 1H), 6.72 (d, 1H), 4.55-4.45 (m, 1H), 3.80-3.65 (m, 1H), 4.65-4.55 (m, 4H), 3.45-3.30 (m, 3H), 3.10-3.00 (m, 1H), 2.70-2.55 (m, 2H), 2.05 (t, 4H), 1.70-1.50 (m, 5H), 1.60-1.48 (m, 1H), 1.46 (s, 9H), 1.35-1.30 (m, 1H)

### Preparation A3-19: tert-butyl (S)-(1-(6-chloro-6′-morpholino-[3,3′-bipyridin]-4-yl)piperidin-3-yl)carbamate

The titled compound was prepared in accordance with the same procedures as in Preparation A3-1, changing the boronic acid reagent to 6-(morpholin-4-yl)pyridin-3-boronic acid pinacol ester. 1H NMR (CDCl₃, 400 MHz) δ 8.28 (d, 1H), 8.00 (s, 1H), 7.71 (d, 1H), 6.86 (s, 1H), 6.72 (d, 1H), 4.55-4.45 (m, 1H), 3.87-3.83 (m, 4H), 3.80-3.65 (m, 1H), 3.59-3.54 (m, 4H), 3.40-3.30 (m, 1H), 3.10-3.00 (m, 1H), 2.70-2.50 (m, 2H), 1.95-1.80 (m, 1H), 1.60-1.50 (m, 1H), 1.45 (s, 9H), 1.35-1.30 (m, 1H)

### Preparation A3-20: tert-butyl (S)-(1-(2-chloro-5-(4-(4-methylpiperazin-1-carbonyl)phenyl)pyridin-4-yl)piperidin-3-yl)carbamate

The titled compound was prepared in accordance with the same procedures as in Preparation A3-1, changing the boronic acid reagent to (4-methylpiperazin-1-yl)-[4-(4,4,5,5-tetramethyl-1,3,2-dioxaborolan-2-yl)phenyl]methanone. 1H NMR (CD₃OD, 400 MHz) δ 7.96 (s, 1H), 7.65 (dd, 2H), 7.56 (dd, 2H), 7.07 (s, 1H), 3.90-3.75 (m, 2H), 3.70-3.50 (m, 2H), 3.50-3.40 (m, 2H), 3.40-3.35 (m, 1H), 3.20-3.10 (m, 1H), 2.75-2.60 (m, 2H), 2.60-2.40 (m, 4H), 2.36 (s, 3H), 1.95-1.85 (m, 1H), 1.65-1.55 (m, 1H), 1.45 (s, 9H), 1.40-1.30 (m, 1H)

### Preparation A3-21: tert-butyl (S)-(1-(2-chloro-5-(1-(2-methoxy-2-methylpropyl)-1H-pyrazol-4-yl)pyridin-4-yl)piperidin-3-yl) carbamate

The titled compound was prepared in accordance with the same procedures as in Preparation A3-1, changing the boronic acid reagent to 1-(2-methoxy-2-methyl-propyl)-4-(4,4,5,5-tetramethyl-1,3,2-dioxaborolan-2-yl)pyrazole. 1H NMR (CD₃OD, 400 MHz) δ 8.18 (s, 1H), 7.84 (s, 1H), 7.76 (s, 1H), 6.91 (s, 1H), 4.66-4.54 (m, 1H), 4.25-4.15 (m, 2H), 3.88-3.76 (m, 1H), 3.75-3.65 (m, 1H), 3.44-3.35 (m, 1H), 3.31 (s, 3H), 3.09-2.98 (m, 1H), 2.71-2.58 (m, 2H), 1.98-1.86 (m, 1H), 1.71-1.52 (m, 1H), 1.51 (s, 9H), 1.43-1.33 (m, 1H), 1.23 (s, 6H)

### Preparation A3-22: tert-butyl (S)-(1-(6-chloro-6′-(2-methoxyethoxy)-[3,3′-bipyridin]-4-yl)piperidin-3-yl)carbamate

The titled compound was prepared in accordance with the same procedures as in Preparation A3-1, changing the boronic acid reagent to 2-(2-methoxyethoxy)-5-(4,4,5,5-tetramethyl-1,3,2-dioxaborolan-2-yl)pyridine. 1H NMR (CD₃OD, 400 MHz) δ 8.26 (s, 1H), 8.06 (s, 1H), 7.82-7.74 (m, 1H), 6.91-6.86 (m, 2H), 4.57-4.52 (m, 2H), 4.52-4.45 (m, 1H), 3.85-3.78 (m, 2H), 3.78-3.63 (m, 2H), 3.52-3.45 (m, 3H), 3.42-3.33 (m, 1H), 3.06-2.94 (m, 1H), 2.73-2.52 (m, 2H), 1.94-1.82 (m, 1H), 1.51 (s, 9H)

### Preparation A3-23: tert-butyl (S)-(1-(2-chloro-5-(1-(2-hydroxy-2-methylpropyl)-1H-pyrazol-4-yl)pyridin-4-yl)piperidin-3-yl) carbamate

The titled compound was prepared in accordance with the same procedures as in Preparation A3-1, changing the boronic acid reagent to [1-(2-hydroxy-2-methyl-prop yl)pyrazol-4-yl]boronic acid pinacol ester. 1H NMR (CD₃OD, 400 MHz) δ 8.24 (s, 1H), 7.99 (s, 1H), 7.84 (s, 1H), 6.94 (s, 1H), 4.48-4.38 (m, 1H), 4.23-4.16 (m, 1H), 4.16 (s, 2H), 3.80-3.63 (m, 1H), 3.62-3.48 (m, 1H), 3.24-3.06 (m, 1H), 2.75-2.62 (m, 1H), 2.29-2.14 (m, 1H), 2.07-1.96 (m, 1H), 1.87-1.77 (m, 1H), 1.77-1.62 (m, 1H), 1.49 (s, 9H)

### Preparation A3-24: tert-butyl (S)-(1-(2-chloro-5-(1-(3-(dimethylamino)propyl)-1H-pyrazol-4-yl)pyridin-4-yl)piperidin-3-yl)carbamate

The titled compound was prepared in accordance with the same procedures as in Preparation A3-1, changing the boronic acid reagent to N,N-dimethyl-3-[4-(4,4,5,5-tetramethyl-1,3,2-dioxaborolan-2-yl)pyrazol-1-yl]propan-1-amine. 1H NMR (CD₃OD, 400 MHz) δ 8.18 (s, 1H), 7.84 (s, 2H), 6.91 (s, 1H), 4.57-4.46 (m, 1H), 4.34 (t, 2H), 3.82-3.67 (m, 1H), 3.47-3.36 (m, 1H), 3.15-3.05 (m, 1H), 2.63-2.57 (m, 2H), 2.57-2.51 (m, 1H), 2.47 (s, 6H), 2.32-2.21 (m, 2H), 2.03-1.91 (m, 1H), 1.78-1.68 (m, 1H), 1.67-1.54 (m, 1H), 1.51 (s, 9H)

### Preparation A3-25: tert-butyl (S)-3-((4-(4-(3-((tert-butoxycarbonyl)amino)piperidin-1-yl)-6-chloropyridin-3-yl)-1H-pyrazol-1-yl)methyl)azetidin-1-carboxylate

The titled compound was prepared in accordance with the same procedures as in Preparation A3-1, changing the boronic acid reagent to tert-butyl 3-[[4-(4,4,5,5-tetramethyl-1,3,2-dioxaborolan-2-yl)pyrazol-1-yl]methyl]azetidin-1-carboxylate. 1H NMR (CD₃OD, 400 MHz) δ 8.17 (s, 1H), 7.81 (s, 1H), 7.76 (s, 1H), 6.91 (s, 1H), 4.55-4.45 (m, 1H), 4.45 (d, 2H), 4.15-4.03 (m, 2H), 3.85-3.76 (m, 2H), 3.76-3.66 (m, 1H), 3.52-3.36 (m, 1H), 3.20-3.02 (m, 2H), 2.67-2.54 (m, 1H), 2.54-2.40 (m, 1H), 2.03-1.89 (m, 1H), 1.78-1.67 (m, 1H), 1.65-1.52 (m, 1H), 1.51 (s, 18H)

### Preparation A3-26: tert-butyl (R)-3-(4-(4-((S)-3-((tert-butoxycarbonyl)amino)piperidin-1-yl)-6-chloropyridin-3-yl)-1H-pyrazol-1-yl)pyrrolidin-1-carboxylate

The titled compound was prepared in accordance with the same procedures as in Preparation A3-1, changing the boronic acid reagent to tert-butyl (R)-3-(4-(4,4,5,5-tetramethyl-1,3,2-dioxaborolan-2-yl)-1H-pyrazol-1-yl)pyrrolidin-1-carboxylate. 1H NMR (CD₃OD, 400 MHz) δ 8.18 (s, 1H), 7.83-7.74 (m, 2H), 6.91 (s, 1H), 5.02-4.90 (m, 1H), 4.55-4.42 (m, 1H), 3.93-3.88 (m, 1H), 3.86-3.77 (m, 1H), 3.77-3.62 (m, 2H), 3.62-3.52 (m, 1H), 3.52-3.41 (m, 1H), 3.16-3.04 (m, 1H), 2.68-2.55 (m, 1H), 2.56-2.39 (m, 3H), 2.04-1.91 (m, 1H), 1.79-1.66 (m, 1H), 1.52 (s, 9H), 1.47 (s, 9H)

### Preparation A3-27: tert-butyl (S)-3-(4-(4-((S)-3-((tert-butoxycarbonyl)amino)piperidin-1-yl)-6-chloropyridin-3-yl)-1H-pyrazol-1-yl)pyrrolidin-1-carboxylate

The titled compound was prepared in accordance with the same procedures as in Preparation A3-1, changing the boronic acid reagent to tert-butyl (S)-3-(4-(4,4,5,5-tetramethyl-1,3,2-dioxaborolan-2-yl)-1H-pyrazol-1-yl)pyrrolidin-1-carboxylate. 1H NMR (CD₃OD, 400 MHz) δ 8.18 (s, 1H), 7.85-7.72 (m, 2H), 6.92 (s, 1H), 5.02-4.91 (m, 1H), 4.56-4.43 (m, 1H), 3.99-3.89 (m, 1H), 3.89-3.77 (m, 1H), 3.77-3.65 (m, 2H), 3.65-3.52 (m, 2H), 3.52-3.39 (m, 1H), 3.17-3.03 (m, 1H), 2.69-2.54 (m, 1H), 2.52-2.38 (m, 3H), 2.02-1.90 (m, 1H), 1.78-1.67 (m, 1H), 1.52 (s, 9H), 1.49 (s, 9H)

### Preparation A3-28: tert-butyl (S)-(1-(6-chloro-6′-((2-(dimethylamino)ethyl)amino)-[3,3′-bipyridin]-4-yl)piperidin-3-yl)carbamate

The titled compound was prepared in accordance with the same procedures as in Preparation A3-1, changing the boronic acid reagent to N′,N′-dimethyl-N-[5-(4,4,5,5-tetramethyl-1,3,2-dioxaborolan-2-yl)-2-pyridyl]ethan-1,2-diamine. 1H NMR (CD₃OD, 400 MHz) δ 8.21 (s, 1H), 8.04 (s, 1H), 7.67 (d, 1H), 6.90 (s, 1H), 6.55 (d, 1H), 5.33-5.18 (m, 1H), 4.64-4.51 (m, 1H), 3.80-3.64 (m, 1H), 3.48-3.38 (m, 2H), 3.38-3.30 (m, 1H), 3.08-2.98 (m, 1H), 2.36 (s, 6H), 1.91-1.85 (m, 1H), 1.52 (s, 9H)

### Preparation A3-29: tert-butyl (S)-4-(4′-(3-((tert-butoxycarbonyl)amino)piperidin-1-yl)-6′-chloro-[3,3′-bipyridin]-6-yl)piperazin-1-carboxylate

The titled compound was prepared in accordance with the same procedures as in Preparation A3-1, changing the boronic acid reagent to tert-butyl 4-[5-(4,4,5,5-tetramethyl-1,3,2-dioxaborolan-2-yl)-2-pyridyl]piperazin-1-carboxylate. 1H NMR (CD₃OD, 400 MHz) δ 8.30 (s, 1H), 8.04 (s, 1H), 7.76 (d, 1H), 6.91 (s, 1H), 6.74 (d, 1H), 4.54-4.42 (m, 1H), 3.79-3.66 (m, 2H), 3.65-3.51 (m, 8H), 3.43-3.33 (m, 3H), 3.11-3.02 (m, 1H), 2.68-2.52 (m, 2H), 1.49 (s, 9H), 1.30 (s, 9H)

### Preparation A3-30: tert-butyl (S)-(1-(2-chloro-5-(1-(1-methoxy-2-methylpropan-2-yl)-1H-pyrazol-4-yl)pyridin-4-yl)piperidin-3-yl)carbamate

The titled compound was prepared in accordance with the same procedures as in Preparation A3-1, changing the boronic acid reagent to 1-(2-methoxy-1,1-dimethyl-ethyl)-4-(4,4,5,5-tetramethyl-1,3,2-dioxaborolan-2-yl)pyrazole. 1H NMR (CD₃OD, 400 MHz) δ 8.19 (s, 1H), 7.95 (s, 1H), 7.80 (s, 1H), 6.87 (s, 1H), 4.67-4.54 (m, 1H), 3.87-3.77 (m, 1H), 3.77-3.68 (m, 1H), 3.68 (s, 2H), 3.43-3.35 (m, 1H), 3.34 (s, 3H), 3.04-2.93 (m, 1H), 2.71-2.60 (m, 2H), 2.05-1.97 (m, 1H), 1.84-1.76 (m, 1H), 1.69 (s, 6H), 1.52 (s, 9H)

### Preparation A3-31: tert-butyl (S)-(1-(2-chloro-5-(1-((3,3-difluorocyclobutyl)methyl)-1H-pyrazol-4-yl)pyridin-4-yl)piperidin-3-yl)carbamate

The titled compound was prepared in accordance with the same procedures as in Preparation A3-1, changing the boronic acid reagent to 1-[(3,3-difluorocyclobutyl)methyl]-4-(4,4,5,5-tetramethyl-1,3,2-dioxaborolan-2-yl)pyrazole. 1H NMR (CD₃OD, 400 MHz) δ 8.17 (s, 1H), 7.82 (s, 1H), 7.76 (s, 1H), 6.92 (s, 1H), 4.55-4.45 (m, 1H), 4.35 (d, 2H), 3.81-3.70 (m, 2H), 3.18-3.07 (m, 1H), 2.83-2.70 (m, 3H), 2.66-2.55 (m, 1H), 2.54-2.39 (m, 3H), 2.04-1.95 (m, 1H), 1.75-1.66 (m, 1H), 1.50 (s, 9H)

### Preparation A3-32: tert-butyl (S)-(1-(2-chloro-5-(1-(2-(dimethylamino)ethyl)-1H-pyrazol-4-yl)pyridin-4-yl)piperidin-3-yl)carbamate

The titled compound was prepared in accordance with the same procedures as in Preparation A3-1, changing the boronic acid reagent to N,N-dimethyl-4-(4,4,5,5-tetramethyl-1,3,2-dioxaborolan-2-yl)-1H-pyrazol-1-ethanamine. 1H NMR (CD₃OD, 400 MHz) δ 8.19 (s, 1H), 7.87 (s, 1H), 7.79 (s, 1H), 6.91 (s, 1H), 4.66-4.51 (m, 1H), 4.37 (t, 2H), 3.88-3.70 (m, 1H), 3.47-3.32 (m, 1H), 3.09-2.98 (m, 1H), 2.88-2.76 (m, 2H), 2.72-2.57 (m, 2H), 2.37 (s, 6H), 2.00-1.87 (m, 1H), 1.77-1.65 (m, 1H), 1.52 (s, 9H), 1.43-1.37 (m, 1H)

### Preparation A3-33: tert-butyl (S)-(1-(2-chloro-5-(1-(fluoromethyl)-1H-pyrazol-4-yl)pyridin-4-yl)piperidin-3-yl)carbamate

The titled compound was prepared in accordance with the same procedures as in Preparation A3-1, changing the boronic acid reagent to 1-(fluoromethyl)-4-(4,4,5,5-tetramethyl-1,3,2-dioxaborolan-2-yl)pyrazole. 1H NMR (CD₃OD, 400 MHz) δ 8.20 (s, 1H), 8.02 (s, 1H), 7.95 (s, 1H), 6.93 (s, 1H), 6.22 (s, 1H), 6.09 (s, 1H), 4.54-4.42 (m, 1H), 3.80-3.66 (m, 2H), 3.50-3.40 (m, 1H), 3.19-3.05 (m, 1H), 2.70-2.56 (m, 1H), 2.55-2.42 (m, 1H), 2.03-1.87 (m, 1H), 1.77-1.67 (m, 1H), 1.52 (s, 9H)

### Preparation A3-34: tert-butyl (S)-4-(4′-(3-((tert-butoxycarbonyl)amino)piperidin-1-yl)-6′-chloro-[3,3′-bipyridin]-6-yl)piperidin-1-carboxylate

The titled compound was prepared in accordance with the same procedures as in Preparation A3-1, changing the boronic acid reagent to tert-butyl 4-[5-(4,4,5,5-tetramethyl-1,3,2-dioxaborolan-2-yl)pyridin-2-yl]piperidin-1-carboxylate. 1H NMR (CD₃OD, 400 MHz) δ 8.70 (s, 1H), 8.09 (s, 1H), 7.86 (d, 1H), 7.28 (d, 1H), 6.95 (s, 1H), 4.49-4.37 (m, 1H), 4.37-4.21 (m, 2H), 3.72-3.55 (m, 1H), 3.43-3.32 (m, 1H), 3.06-2.95 (m, 1H), 2.95-2.79 (m, 3H), 2.67-2.59 (m, 1H), 2.59-2.47 (m, 1H), 2.05-1.94 (m, 2H), 1.94-1.85 (m, 1H), 1.85-1.71 (m, 2H), 1.53 (s, 9H), 1.47 (s, 9H)

### Preparation A3-35: tert-butyl (S)-(1-(6-chloro-6′-(2-(dimethylamino)ethoxy)-[3,3′-bipyridin]-4-yl)piperidin-3-yl)carbamate

The titled compound was prepared in accordance with the same procedures as in Preparation A3-1, changing the boronic acid reagent to N,N-dimethyl-2-[5-(4,4,5,5-tetramethyl-1,3,2-dioxaborolan-2-yl)pyridin-2-yl]oxyethanamine. 1H NMR (CD₃OD, 400 MHz) δ 8.26 (s, 1H), 8.06 (s, 1H), 7.80-7.72 (m, 1H), 6.94-6.83 (m, 2H), 4.56 (t, 2H), 3.75-3.62 (m, 1H), 3.40-3.29 (m, 1H), 3.04-2.96 (m, 1H), 2.83 (t, 2H), 2.43 (s, 6H), 1.92-1.83 (m, 1H), 1.80-1.63 (m, 3H), 1.63-1.52 (m, 1H), 1.52 (s, 9H)

### Preparation A3-36: tert-butyl (S)-(1-(6-chloro-6′-(3-ethylureido)-[3,3′-bipyridin]-4-yl)piperidin-3-yl)carbamate

The titled compound was prepared in accordance with the same procedures as in Preparation A3-1, changing the boronic acid reagent to 1-ethyl-3-[5-(4,4,5,5-tetra methyl-1,3,2-dioxaborolan-2-yl)pyridin-2-yl]urea. 1H NMR (CD₃OD, 400 MHz) S 9.32 (s, 1H), 8.33 (s, 1H), 8.07 (s, 1H), 7.84 (d, 1H), 6.95 (s, 1H), 4.52-4.39 (m, 1H), 3.76-3.60 (m, 1H), 3.53 (q, 2H), 3.45-3.37 (m, 1H), 3.12-2.96 (m, 1H), 2.66-2.51 (m, 2H), 2.00-1.86 (m, 1H), 1.64-1.57 (m, 2H), 1.54 (s, 9H), 0.92 (t, 3H)

### Preparation A3-37: tert-butyl N-[(3S)-1-[2-chloro-5-[4-(morpholin-4-carbonyl)phenyl]-4-pyridyl]-3-piperidyl]carbamate

The titled compound was prepared in accordance with the same procedures as in Preparation A3-1, changing the boronic acid reagent to 4-(morpholin-4-carbonyl)phenyl boronic acid pinacol ester. 1H NMR (CDCl₃, 400 MHz) δ 8.03 (s, 1H), 7.59-7.56 (m, 2H), 7.54-7.51 (m, 2H), 6.91 (s, 1H), 3.90-3.55 (m, 1H), 3.45-3.35 (m, 1H), 3.05-2.95 (m, 1H), 2.65-2.55 (m, 2H), 1.95-1.85 (m, 1H), 1.46 (s, 9H), 1.46-1.40 (m, 2H)

### Preparation A3-38: tert-butyl N-[(3S)-1-[2-chloro-5-(4-methyl-2,3-dihydropyrido[3,2-b][1,4]oxazin-7-yl)-4-pyridyl]-3-piperidyl]carbamate

The titled compound was prepared in accordance with the same procedures as in Preparation A3-1, changing the boronic acid reagent to 4-methyl-7-(4,4,5,5-tetramethyl-1,3,2-dioxaborolan-2-yl)-2,3-dihydropyrido[3,2-b][1,4]oxazine. 1H NMR (CDCl₃, 400 MHz) δ 8.02 (s, 1H), 7.83 (s, 1H), 7.14 (s, 1H), 6.84 (s, 1H), 4.55-4.40 (m, 1H), 4.40-4.25 (m, 2H), 3.75-3.65 (m, 1H), 3.55-3.45 (m, 2H), 3.41 (d, 1H), 3.19 (s, 3H), 3.15-3.05 (m, 1H), 2.70-2.50 (m, 2H), 1.95-1.85 (m, 1H), 1.55-1.48 (m, 1H), 1.46 (s, 9H), 1.35-1.25 (m, 1H)

### Preparation A3-39: tert-butyl N-[(3S)-1-[2-chloro-5-(4-methyl-2,3-dihydro-1,4-benzoxazin-7-yl)-4-pyridyl]-3-piperidyl]carbamate

The titled compound was prepared in accordance with the same procedures as in Preparation A3-1, changing the boronic acid reagent to 4-methyl-7-(4,4,5,5-tetramethyl-1,3,2-dioxaborolan-2-yl)-3,4-dihydro-2H-1,4-benzoxazine. 1H NMR (CDCl₃, 400 MHz) δ 8.02 (s, 1H), 6.97 (d, 1H), 6.92 (s, 1H), 6.82 (s, 1H), 6.72 (d, 1H), 4.60-4.50 (m, 1H), 4.40-4.30 (m, 2H), 3.80-3.70 (m, 1H), 3.40-3.25 (m, 3H), 3.05-2.96 (m, 1H), 2.95 (s, 3H), 2.70-2.60 (m, 2H), 1.90-1.80 (m, 1H), 1.55-1.40 (m, 10H), 1.40-1.35 (m, 1H)

### Preparation A3-40: tert-butyl N-[(3S)-1-[2-chloro-5-(2-oxo-1,4-dihydro-3,1-benzoxazin-6-yl)-4-pyridyl]-3-piperidyl]carbamate

The titled compound was prepared in accordance with the same procedures as in Preparation A3-1, changing the boronic acid reagent to 6-(4,4,5,5-tetramethyl-1,3,2-dioxaborolan-2-yl)-1,4-dihydro-2H-benzo[d][1,3]oxazin-2-one. 1H NMR (CDCl₃, 400 MHz) δ 8.00 (s, 1H), 7.41 (d, 1H), 7.13 (s, 1H), 6.90-6.85 (m, 1H), 6.67 (d, 1H), 5.45-5.35 (m, 2H), 4.45-4.35 (m, 1H), 3.70-3.60 (m, 1H), 3.35-3.30 (m, 1H), 3.10-3.00 (m, 1H), 2.64 (t, 1H), 2.51 (t, 1H), 1.95-1.85 (m, 1H), 1.45 (s, 9H), 1.45-1.35 (m, 1H), 1.35-1.30 (m, 1H)

### Preparation A3-41: tert-butyl N-[(3S)-1-[2-chloro-5-[4-(2-oxopyrrolidin-1-yl)phenyl]-4-pyridyl]-3-piperidyl]carbamate

The titled compound was prepared in accordance with the same procedures as in Preparation A3-1, changing the boronic acid reagent to 4-(2-oxo-1-pyrrolidinyl)phenyl boronic acid pinacol ester. 1H NMR (CDCl₃, 400 MHz) δ 8.03 (s, 1H), 7.75 (d, 2H), 7.50 (d, 2H), 6.87 (s, 1H), 4.55-4.45 (m, 1H), 3.95 (t, 2H), 3.75-3.65 (m, 1H), 3.35-3.25 (m, 1H), 3.05-2.95 (m, 1H), 2.70-2.60 (m, 3H), 2.30-2.10 (m, 3H), 1.90-1.80 (m, 1H), 1.46 (s, 9H), 1.46-1.30 (m, 2H)

### Preparation A3-42: tert-butyl N-[(3S)-1-[2-chloro-5-(3-oxo-4H-1,4-benzoxazin-7-yl)-4-pyridyl]-3-piperidyl]carbamate

The titled compound was prepared in accordance with the same procedures as in Preparation A3-1, changing the boronic acid reagent to 7-(4,4,5,5-tetramethyl-1,3,2-dioxaborolan-2-yl)-2H-benzo[b][1,4]oxazin-3(4H)-one. 1H NMR (CDCl₃, 400 MHz) δ 8.02 (s, 1H), 7.68 (s, 1H), 7.16 (s, 1H), 7.10-7.05 (m, 1H), 6.90-6.80 (m, 2H), 4.73 (q, 2H), 4.45-4.35 (m, 1H), 3.70-3.60 (m, 1H), 3.40-3.30 (m, 1H), 3.15-3.05 (m, 1H), 2.65 (t, 1H), 2.47 (t, 1H), 1.95-1.85 (m, 1H), 1.70-1.60 (m, 1H), 1.44 (s, 9H), 1.35-1.30 (m, 1H)

### Preparation A3-43: tert-butyl N-[(3S)-1-[2-chloro-5-(4-methyl-3-oxo-1,4-benzoxazin-7-yl)-4-pyridyl]-3-piperidyl]carbamate

The titled compound was prepared in accordance with the same procedures as in Preparation A3-1, changing the boronic acid reagent to 4-methyl-3-oxo-3,4-dihydro-2H-benzo[b][1,4]oxazin-7-boronic acid pinacol ester. 1H NMR (CDCl₃, 400 MHz) δ 8.03 (s, 1H), 7.20-7.15 (m, 2H), 7.10-7.05 (m, 1H), 6.86 (s, 1H), 4.72 (q, 2H), 4.45-4.35 (m, 1H), 3.70-3.60 (m, 1H), 3.43 (s, 3H), 3.40-3.30 (m, 1H), 3.15-3.05 (m, 1H), 2.65 (t, 1H), 2.47 (t, 1H), 1.95-1.85 (m, 1H), 1.70-1.60 (m, 1H), 1.42 (s, 9H), 1.30-1.20 (m, 1H)

### Preparation A3-44: tert-butyl (S)-(1-(2-chloro-5-(8-methyl-2-oxo-1,2,3,4-tetrahydroquinolin-6-yl)pyridin-4-yl)piperidin-3-yl)carbamate

The titled compound was prepared in accordance with the same procedures as in Preparation A3-1, changing the boronic acid reagent to 8-methyl-6-(4,4,5,5-tetramethyl-1,3,2-dioxaborolan-2-yl)-3,4-dihydroquinolin-2(1H)-one. 1H NMR (CDCl₃, 400 MHz) δ 7.99 (s, 1H), 7.15 (s, 1H), 6.83 (s, 1H), 6.54 (s, 1H), 4.55-4.45 (m, 1H), 3.75-3.60 (m, 1H), 3.35-3.25 (m, 1H), 3.10-2.95 (m, 3H), 2.90 (t, 1H), 2.80-2.50 (m, 3H), 2.28 (s, 3H), 1.90-1.80 (m, 1H), 1.50-1.40 (m, 10H), 1.40-1.30 (m, 1H)

### Preparation A3-45: tert-butyl (S)-(1-(2-chloro-5-(1-methyl-2-oxo-1,2,3,4-tetrahydroquinolin-6-yl)pyridin-4-yl)piperidin-3-yl)carbamate

The titled compound was prepared in accordance with the same procedures as in Preparation A3-1, changing the boronic acid reagent to 1-methyl-6-(4,4,5,5-tetramethyl-1,3,2-dioxaborolan-2-yl)-3,4-dihydroquinolin-2(1H)-one. 1H NMR (CDCl₃, 400 MHz) δ 8.01 (s, 1H), 7.30 (s, 1H), 6.90-6.80 (m, 2H), 6.80-6.70 (m, 1H), 4.50-4.40 (m, 1H), 3.70-3.60 (m, 1H), 3.40 (s, 3H), 3.35-3.30 (m, 1H), 3.10-2.95 (m, 3H), 2.85 (t, 1H), 2.80-2.60 (m, 3H), 1.90-1.80 (m, 1H), 1.50-1.40 (m, 10H), 1.40-1.30 (m, 1H)

### Preparation A3-46: tert-butyl 4-[4-[6-chloro-4-[(3S)-3-(tert-butoxycarbonylamino)-1-piperidyl]-3-pyridyl]benzoyl]piperazin-1-carboxylate

The titled compound was prepared in accordance with the same procedures as in Preparation A3-1, changing the boronic acid reagent to tert-butyl 4-[4-(4,4,5,5-tetramethyl-1,3,2-dioxaborolan-2-yl)benzoyl]piperazin-1-carboxylate. 1H NMR (CDCl₃, 400 MHz) δ 8.03 (s, 1H), 7.60-7.55 (m, 2H), 7.55-7.50 (m, 2H), 6.90 (s, 1H), 4.55-4.45 (m, 1H), 3.75-3.60 (m, 4H), 3.60-3.40 (m, 5H), 3.05-2.95 (m, 1H), 2.70-2.50 (m, 2H), 1.95-1.85 (m, 1H), 1.55-1.45 (m, 10H), 1.45-1.40 (m, 2H)

### Preparation A3-47: tert-butyl N-[(3S)-1-[2-chloro-5-[1-(2,2,6,6-tetramethyltetrahydropyran-4-yl)pyrazol-4-yl]-4-pyridyl]-3-piperidyl]carbamate

The titled compound was prepared in accordance with the same procedures as in Preparation A3-1, changing the boronic acid reagent to 4-(4,4,5,5-tetramethyl-1,3,2-dioxaborolan-2-yl)-1-(2,2,6,6-tetramethyltetrahydropyran-4-yl)pyrazole. 1H NMR (MeOD, 400 MHz) δ 8.13 (s, 1H), 8.09 (s, 1H), 7.84 (s, 1H), 7.03 (s, 1H), 4.90-4.80 (m, 1H), 3.65-3.50 (m, 1H), 3.50-3.40 (m, 1H), 3.30-3.20 (m, 1H), 2.64 (t, 1H), 2.41 (t, 1H), 2.25-2.10 (m, 2H), 2.00-1.85 (m, 2H), 1.85-1.70 (m, 1H), 1.70-1.55 (m, 1H), 1.50-1.35 (m, 15H), 1.31 (s, 6H), 1.25-1.15 (m, 2H)

### Preparation A3-48: tert-butyl N-[(3S)-1-[2-chloro-5-(2-methyl-3-oxo-4H-1,4-benzoxazin-6-yl)-4-pyridyl]-3-piperidyl]carbamate

The titled compound was prepared in accordance with the same procedures as in Preparation A3-1, changing the boronic acid reagent to 3,4-dihydro-2-methyl-6-(4,4,5,5-tetramethyl-1,3,2-dioxaborolan-2H-1,4-benzoxazine. 1H NMR (CDCl₃, 400 MHz) δ 8.03 (s, 1H), 7.15-7.05 (m, 2H), 7.05-6.95 (m, 1H), 6.87 (s, 1H), 3.75-3.60 (m, 1H), 3.35-3.25 (m, 1H), 3.05-2.90 (m, 1H), 2.70-2.50 (m, 2H), 1.90-1.80 (m, 1H), 1.70-1.65 (m, 1H), 1.61 (s, 3H), 1.60-1.50 (m, 2H), 1.46 (s, 9H)

### Preparation A3-49: tert-butyl N-[(3S)-1-[2-chloro-5-(4-methyl-3-oxo-pyrido[3,2-b]oxazin-7-yl)-4-pyridyl]-3-piperidyl]carbamate

The titled compound was prepared in accordance with the same procedures as in Preparation A3-1, changing the boronic acid reagent to 4-methyl-7-(4,4,5,5-tetramethyl-1,3,2-dioxaborolan-2-yl)pyrido[3,2-b][1,4]oxazin-3-one. 1H NMR (CDCl₃, 400 MHz) δ 8.05-8.00 (m, 2H), 6.88 (s, 1H), 6.69 (s, 1H), 4.95-4.85 (m, 1H), 4.80-4.70 (m, 1H), 4.35-4.25 (m, 1H), 3.53 (s, 3H), 3.25-3.05 (m, 3H), 2.70-2.60 (m, 1H), 2.30-2.20 (m, 1H), 2.00-1.90 (m, 1H), 1.85-1.75 (m, 1H), 1.75-1.65 (m, 1H), 1.60-1.50 (m, 1H), 1.48 (s, 9H)

### Preparation A3-50: tert-butyl N-[(3S)-1-[2-chloro-5-(3-oxo-4H-pyrido[3,2-b][1,4]oxazin-7-yl)-4-pyridyl]-3-piperidyl]carbamate

The titled compound was prepared in accordance with the same procedures as in Preparation A3-1, changing the boronic acid reagent to 7-(4,4,5,5-tetramethyl-1,3,2-dioxaborolan-2-yl)-4H-pyrido[3,2-b][1,4]oxazin-3-one. 1H NMR (CDCl₃, 400 MHz) δ 9.41 (s, 1H), 8.15-8.05 (m, 2H), 7.48 (s, 1H), 6.89 (s, 1H), 4.95-4.85 (m, 1H), 4.80-4.70 (m, 1H), 4.40-4.30 (m, 1H), 3.60-3.50 (m, 1H), 3.45-3.30 (m, 1H), 3.25-3.15 (m, 1H), 2.67 (t, 1H), 2.29 (t, 1H), 2.00-1.90 (m, 1H), 1.85-1.70 (m, 1H), 1.70-1.60 (m, 1H), 1.38 (s, 9H)

### Preparation A3-51: tert-butyl N-[(3S)-1-[2-chloro-5-(7-oxo-6,8-dihydro-5H-1,8-naphthyridin-3-yl)-4-pyridyl]-3-piperidyl]carbamate

The titled compound was prepared in accordance with the same procedures as in Preparation A3-1, changing the boronic acid reagent to 6-(4,4,5,5-tetramethyl-1,3,2-dioxaborolan-2-yl)-3,4-dihydro-1H-1,8-naphthyridin-2-one. 1H NMR (CDCl₃, 400 MHz) δ 8.27 (s, 1H), 8.20 (s, 1H), 8.04 (s, 1H), 7.70 (s, 1H), 6.90 (s, 1H), 4.45-4.35 (m, 1H), 3.60-3.50 (m, 1H), 3.45-3.35 (m, 1H), 3.20-3.10 (m, 1H), 3.10-3.00 (m, 2H), 2.90-2.70 (m, 2H), 2.67 (t, 1H), 2.40 (t, 1H), 2.00-1.90 (m, 1H), 1.75-1.65 (m, 1H), 1.60-1.50 (m, 1H), 1.42 (s, 9H)

### Preparation A3-52: tert-butyl N-[(3S)-1-[2-chloro-5-[1-(oxetan-3-yl)pyrazol-4-yl]-4-pyridyl]-3-piperidyl]carbamate

The titled compound was prepared in accordance with the same procedures as in Preparation A3-1, changing the boronic acid reagent to 1-(oxetan-3-yl)-4-(4,4,5,5-tetramethyl-1,3,2-dioxaborolan-2-yl)pyrazole. 1H NMR (CDCl₃, 400 MHz) δ 8.16 (s, 1H), 7.93 (s, 1H), 7.88 (s, 1H), 6.89 (s, 1H), 5.60-5.50 (m, 1H), 5.20-5.05 (m, 4H), 4.50-4.40 (m, 1H), 3.75-3.65 (m, 1H), 3.50-3.40 (m, 1H), 3.20-3.10 (m, 1H), 2.65 (t, 1H), 2.39 (t, 1H), 2.00-1.90 (m, 1H), 1.80-1.70 (m, 1H), 1.70-1.60 (m, 1H), 1.44 (s, 9H)

### Preparation A3-53: methyl 2-[4-[6-chloro-4-[(3S)-3-(tert-butoxycarbonylamino)-1-piperidyl]-3-pyridyl]pyrazol-1-yl]-2-methyl-propanoate

The titled compound was prepared in accordance with the same procedures as in Preparation A3-1, changing the boronic acid reagent to methyl 2-methyl-2-[4-(4,4,5,5-tetramethyl-1,3,2-dioxaborolan-2-yl)pyrazol-1-yl]propanoate. 1H NMR (CDCl₃, 400 MHz) δ 8.16 (s, 1H), 7.93 (s, 1H), 7.81 (s, 1H), 6.87 (s, 1H), 4.65-4.55 (m, 1H), 3.75 (s, 3H), 3.45-3.35 (m, 1H), 3.10-3.00 (m, 1H), 2.70-2.55 (m, 2H), 2.00-1.90 (m, 6H), 1.70-1.60 (m, 1H), 1.60-1.50 (m, 1H), 1.46 (s, 9H), 1.46-1.25 (m, 2H)

### Preparation A3-54: tert-butyl N-[(3S)-1-[5-(4-acetamidophenyl)-2-chloro-4-pyridyl]-3-piperidyl]carbamate

The titled compound was prepared in accordance with the same procedures as in Preparation A3-1, changing the boronic acid reagent to 4-acetamidobenzeneboronic acid. 1H NMR (CDCl₃, 400 MHz) δ 8.03 (s, 1H), 7.65-7.60 (m, 2H), 7.50-7.40 (m, 2H), 7.24 (s, 1H), 6.87 (s, 1H), 4.65-4.55 (m, 1H), 3.75-3.65 (m, 1H), 3.35-3.25 (m, 1H), 3.00-2.90 (m, 1H), 2.70-2.55 (m, 2H), 2.23 (s, 3H), 1.90-1.80 (m, 1H), 1.47 (s, 9H), 1.45-1.30 (m, 2H)

### Preparation A3-55: tert-butyl N-[(3S)-1-[2-chloro-5-[3-(dimethylcarbamoyl)phenyl]-4-pyridyl]-3-piperidyl]carbamate

The titled compound was prepared in accordance with the same procedures as in Preparation A3-1, changing the boronic acid reagent to 3-(dimethylcarbamoyl)phenyl boronic acid. 1H NMR (CDCl₃, 400 MHz) δ 8.06 (s, 1H), 7.67 (s, 1H), 7.60-7.45 (m, 2H), 7.45-7.35 (m, 1H), 6.89 (s, 1H), 4.95-4.85 (m, 1H), 3.80-3.70 (m, 1H), 3.35-3.25 (m, 1H), 3.15 (s, 3H), 3.13 (s, 3H), 2.90-2.80 (m, 1H), 2.80-2.70 (m, 1H), 2.70-2.60 (m, 1H), 1.85-1.75 (m, 1H), 1.50-1.30 (m, 11H)

### Preparation A3-56: tert-butyl N-[(3S)-1-[2-chloro-5-[3-methyl-4-(trifluoromethoxy)phenyl]-4-pyridyl]-3-piperidyl]carbamate

The titled compound was prepared in accordance with the same procedures as in Preparation A3-1, changing the boronic acid reagent to 3-methyl-4-trifluoromethoxy phenylboronic acid. 1H NMR (CDCl₃, 400 MHz) δ 8.03 (s, 1H), 7.39 (s, 1H), 7.35-7.30 (m, 2H), 6.88 (s, 1H), 4.55-4.45 (m, 1H), 3.80-3.65 (m, 1H), 3.35-3.25 (m, 1H), 2.95-2.85 (m, 1H), 2.70-2.60 (m, 2H), 2.40 (s, 3H), 1.90-1.80 (m, 1H), 1.47 (s, 9H), 1.47-1.30 (m, 2H)

### Preparation A3-57: tert-butyl N-[(3S)-1-[2-chloro-5-(4-fluoro-3-methyl-phenyl)-4-pyridyl]-3-piperidyl]carbamate

The titled compound was prepared in accordance with the same procedures as in Preparation A3-1, changing the boronic acid reagent to 4-fluoro-3-methylphenylboronic acid. 1H NMR (CDCl₃, 400 MHz) δ 8.01 (s, 1H), 7.35-7.25 (m, 2H), 7.10 (t, 1H), 6.87 (s, 1H), 4.60-4.45 (m, 1H), 3.75-3.65 (m, 1H), 3.25-3.15 (m, 1H), 2.95-2.85 (m, 1H), 2.75-2.65 (m, 2H), 2.36 (s, 3H), 1.85-1.75 (m, 1H), 1.47 (s, 9H), 1.47-1.35 (m, 2H)

### Preparation A3-58: tert-butyl 4-[4-[6-chloro-4-[(3S)-3-(tert-butoxycarbonylamino)-1-piperidyl]-3-pyridyl]pyrazol-1-yl]piperidin-1-carboxylate

The titled compound was prepared in accordance with the same procedures as in Preparation A3-1, changing the boronic acid reagent to tert-butyl 4-[4-(4,4,5,5-tetramethyl-1,3,2-dioxaborolan-2-yl)pyrazol-1-yl]piperidin-1-carboxylate. 1H NMR (CDCl₃, 400 MHz) δ 8.15 (s, 1H), 7.85-7.75 (m, 2H), 6.87 (s, 1H), 4.55-4.45 (m, 1H), 4.40-4.20 (m, 2H), 3.80-3.65 (m, 1H), 3.50-3.40 (m, 1H), 3.15-3.05 (m, 1H), 3.00-2.95 (m, 2H), 2.65-2.50 (m, 1H), 2.50-2.45 (m, 1H), 2.30-2.15 (m, 2H), 2.05-1.90 (m, 2H), 1.75-1.65 (m, 1H), 1.65-1.55 (m, 1H), 1.49 (s, 9H), 1.46 (s, 9H), 1.46-1.25 (m, 2H)

### Preparation A3-59: tert-butyl N-[(3S)-1-[2-chloro-5-(4-isopropoxy-3,5-dimethyl-phenyl)-4-pyridyl]-3-piperidyl]carbamate

The titled compound was prepared in accordance with the same procedures as in Preparation A3-1, changing the boronic acid reagent to 3,5-dimethyl-4-isopropoxyphenylboronic acid. 1H NMR (CDCl₃, 400 MHz) δ 8.03 (s, 1H), 7.12 (s, 2H), 6.82 (s, 1H), 4.70-4.55 (m, 1H), 4.35-4.20 (m, 1H), 3.85-3.70 (m, 1H), 3.30-3.20 (m, 1H), 3.10-3.00 (m, 1H), 2.90-2.80 (m, 1H), 2.34 (s, 6H), 2.00-1.85 (m, 1H), 1.85-1.70 (m, 1H), 1.48 (s, 9H), 1.35 (d, 6H), 1.35-1.25 (m, 2H)

### Preparation A3-60: tert-butyl N-[(3S)-1-[2-chloro-5-(4-methoxy-3,5-dimethyl-phenyl)-4-pyridyl]-3-piperidyl]carbamate

The titled compound was prepared in accordance with the same procedures as in Preparation A3-1, changing the boronic acid reagent to 4-methoxy-3,5-dimethylphenylboronic acid. 1H NMR (CDCl₃, 400 MHz) δ 8.02 (s, 1H), 7.13 (s, 2H), 6.83 (s, 1H), 4.65-4.55 (m, 1H), 3.79 (s, 3H), 3.79-3.70 (m, 1H), 3.30-3.20 (m, 1H), 2.90-2.80 (m, 1H), 2.80-2.70 (m, 1H), 2.70-2.60 (m, 1H), 2.36 (s, 6H), 1.80-1.70 (m, 1H), 1.47 (s, 9H), 1.47-1.35 (m, 2H)

### Preparation A3-61: tert-butyl N-[(3S)-1-[2-chloro-5-(4-morpholinophenyl)-4-pyridyl]-3-piperidyl]carbamate

The titled compound was prepared in accordance with the same procedures as in Preparation A3-1, changing the boronic acid reagent to (4-morpholinophenyl)boronic acid. 1H NMR (CDCl₃, 400 MHz) δ 8.02 (s, 1H), 7.41 (d, 2H), 6.99 (d, 2H), 6.85 (s, 1H), 4.60-4.50 (m, 1H), 3.95-3.90 (m, 4H), 3.90-3.85 (m, 1H), 3.80-3.70 (m, 1H), 3.35-3.30 (m, 1H), 3.30-3.20 (m, 4H), 3.10-3.00 (m, 1H), 3.00-2.90 (m, 1H), 2.70-2.60 (m, 2H), 1.90-1.80 (m, 1H), 1.60-1.55 (m, 1H), 1.48 (s, 9H), 1.48-1.30 (m, 1H)

### Preparation A3-62: tert-butyl N-[(3S)-1-[2-chloro-5-(3-methyl-4-morpholino-phenyl)-4-pyridyl]-3-piperidyl]carbamate

The titled compound was prepared in accordance with the same procedures as in Preparation A3-1, changing the boronic acid reagent to 3-methyl-4-morpholinophenyl boronic acid. 1H NMR (CDCl₃, 400 MHz) δ 8.02 (s, 1H), 7.35-7.25 (m, 2H), 7.10 (d, 2H), 6.84 (s, 1H), 4.60-4.50 (m, 1H), 3.95-3.85 (m, 4H), 3.75-3.65 (m, 1H), 3.30-3.20 (m, 1H), 3.05-2.95 (m, 4H), 2.95-2.90 (m, 1H), 2.75-2.60 (m, 2H), 2.39 (s, 3H), 1.85-1.75 (m, 1H), 1.60-1.50 (m, 1H), 1.46 (s, 9H), 1.46-1.35 (m, 1H)

### Preparation A3-63: tert-butyl N-[(3S)-1-[2-chloro-5-[4-[4-(2-hydroxyethyl)piperazin-1-carbonyl]phenyl]-4-pyridyl]-3-piperidyl]carbamate

The titled compound was prepared in accordance with the same procedures as in Preparation A3-1, changing the boronic acid reagent to [4-[4-(2-hydroxyethyl)piperazin-1-carbonyl]phenyl]boronic acid. 1H NMR (CDCl₃, 400 MHz) δ 8.04 (s, 1H), 7.60-7.55 (m, 2H), 7.55-7.50 (m, 2H), 6.90 (s, 1H), 4.55-4.45 (m, 1H), 3.90-3.45 (m, 9H), 3.45-3.35 (m, 1H), 3.05-2.95 (m, 1H), 2.70-2.60 (m, 4H), 2.60-2.40 (m, 6H), 1.95-1.80 (m, 1H), 1.47 (s, 9H), 1.47-1.30 (m, 2H)

### Preparation A3-64: tert-butyl N-[(3S)-1-[2-chloro-5-[4-[((trans)4-hydroxycyclohexyl)carbamoyl]phenyl]-4-pyridyl]-3-piperidyl]carbamate

The titled compound was prepared in accordance with the same procedures as in Preparation A3-1, changing the boronic acid reagent to 4-(trans-4-hydroxycyclohexylcarbamoyl)phenylboronic acid. 1H NMR (CDCl₃, 400 MHz) δ 8.03 (s, 1H), 7.90-7.80 (m, 2H), 7.60-7.50 (m, 2H), 6.90 (s, 1H), 6.15-6.05 (m, 1H), 4.50-4.40 (m, 1H), 4.10-3.90 (m, 2H), 3.75-3.60 (m, 2H), 3.35-3.25 (m, 1H), 3.05-2.95 (m, 1H), 2.70-2.60 (m, 1H), 2.60-2.50 (m, 1H), 2.25-2.15 (m, 3H), 2.15-2.00 (m, 3H), 1.95-1.80 (m, 1H), 1.60-1.50 (m, 1H), 1.44 (s, 9H), 1.44-1.30 (m, 2H)

### Preparation A3-65: tert-butyl N-[(3S)-1-[2-chloro-5-[4-(dimethylcarbamoyl)-3-fluoro-phenyl]-4-pyridyl]-3-piperidyl]carbamate

The titled compound was prepared in accordance with the same procedures as in Preparation A3-1, changing the boronic acid reagent to 4-(dimethylcarbamoyl)-3-fluorophenylboronic acid. 1H NMR (CDCl₃, 400 MHz) δ 8.03 (s, 1H), 7.51 (t, 1H), 7.40-7.35 (m, 1H), 7.35-7.25 (m, 1H), 6.91 (s, 1H), 4.55-4.45 (m, 1H), 3.75-3.65 (m, 1H), 3.50-3.40 (m, 1H), 3.18 (s, 3H), 3.05-2.95 (m, 4H), 2.65-2.55 (m, 2H), 1.95-1.85 (m, 1H), 1.55-1.50 (m, 1H), 1.46 (s, 9H), 1.46-1.30 (m, 1H)

### Preparation A3-66: tert-butyl N-[(3S)-1-[5-(3-acetylphenyl)-2-chloro-4-pyridyl]-3-piperidyl]carbamate

The titled compound was prepared in accordance with the same procedures as in Preparation A3-1, changing the boronic acid reagent to 3-acetylphenylboronic acid. 1H NMR (CDCl₃, 400 MHz) δ 8.14 (s, 1H), 8.07 (s, 1H), 7.97 (d, 1H), 7.70 (d, 1H), 7.58 (t, 1H), 6.91 (s, 1H), 4.65-4.55 (m, 1H), 3.75-3.65 (m, 1H), 3.30-3.20 (m, 1H), 2.95-2.85 (m, 1H), 2.75-2.65 (m, 2H), 2.67 (s, 3H), 1.81 (t, 1H), 1.60-1.50 (m, 1H), 1.45 (s, 9H), 1.45-1.35 (m, 1H)

### Preparation A3-67: tert-butyl N-[(3S)-1-[2-chloro-5-(3-chloro-4-fluoro-phenyl)-4-pyridyl]-3-piperidyl]carbamate

The titled compound was prepared in accordance with the same procedures as in Preparation A3-1, changing the boronic acid reagent to 3-chloro-4-fluorophenylboronic acid. 1H NMR (CDCl₃, 400 MHz) δ 8.02 (s, 1H), 7.57 (d, 1H), 7.45-7.35 (m, 1H), 7.25-7.15 (m, 1H), 6.89 (s, 1H), 4.55-4.45 (m, 1H), 3.75-3.65 (m, 1H), 3.35-3.25 (m, 1H), 3.00-2.90 (m, 1H), 2.70-2.60 (m, 2H), 1.90-1.80 (m, 1H), 1.55-1.50 (m, 1H), 1.47 (s, 9H), 1.47-1.35 (m, 1H)

### Preparation A3-68: tert-butyl N-[(3S)-1-[2-chloro-5-[4-fluoro-3-(trifluoromethyl)phenyl]-4-pyridyl]-3-piperidyl]carbamate

The titled compound was prepared in accordance with the same procedures as in Preparation A3-1, changing the boronic acid reagent to 4-fluoro-3-(trifluoromethyl)phenylboronic acid. 1H NMR (CDCl₃, 400 MHz) δ 8.03 (s, 1H), 7.79 (d, 1H), 7.75-7.65 (m, 1H), 7.33 (t, 1H), 6.91 (s, 1H), 4.55-4.40 (m, 1H), 3.75-3.60 (m, 1H), 3.35-3.25 (m, 1H), 3.00-2.90 (m, 1H), 2.70-2.55 (m, 2H), 1.90-1.80 (m, 1H), 1.60-1.50 (m, 1H), 1.46 (s, 9H), 1.46-1.30 (m, 1H)

### Preparation A3-69: tert-butyl N-[(3S)-1-[2-chloro-5-(3,4-difluorophenyl)-4-pyridyl]-3-piperidyl]carbamate

The titled compound was prepared in accordance with the same procedures as in Preparation A3-1, changing the boronic acid reagent to 3,4-difluorophenylboronic acid. 1H NMR (CDCl₃, 400 MHz) δ 8.01 (s, 1H), 7.40-7.30 (m, 1H), 7.25-7.20 (m, 1H), 7.20-7.15 (m, 1H), 6.90 (s, 1H), 4.50-4.40 (m, 1H), 3.75-3.60 (m, 1H), 3.35-3.25 (m, 1H), 3.05-2.95 (m, 1H), 2.75-2.60 (m, 2H), 1.95-1.80 (m, 1H), 1.65-1.55 (m, 1H), 1.47 (s, 9H), 1.47-1.30 (m, 1H)

### Preparation A3-70: tert-butyl N-[(3S)-1-[2-chloro-5-[3-(dimethylamino)-4-fluoro-phenyl]-4-pyridyl]-3-piperidyl]carbamate

The titled compound was prepared in accordance with the same procedures as in Preparation A3-1, changing the boronic acid reagent to (3-(dimethylamino)-4-fluoro phenyl)boronic acid. 1H NMR (CDCl₃, 400 MHz) δ 8.03 (s, 1H), 7.15-7.05 (m, 1H), 7.00-6.95 (m, 1H), 6.95-6.85 (m, 1H), 6.86 (s, 1H), 4.65-4.55 (m, 1H), 3.75-3.65 (m, 1H), 3.30-3.20 (m, 1H), 2.91 (s, 6H), 2.91-2.85 (m, 1H), 2.75-2.65 (m, 2H), 1.85-1.75 (m, 1H), 1.60-1.50 (m, 1H), 1.47 (s, 9H), 1.47-1.35 (m, 1H)

### Preparation A3-71: tert-butyl N-[(3S)-1-[2-chloro-5-[1-[(3S)-tetrahydrofuran-3-yl]pyrazol-4-yl]-4-pyridyl]-3-piperidyl]carbamate

The titled compound was prepared in accordance with the same procedures as in Preparation A3-1, changing the boronic acid reagent to (S)-1-(tetrahydrofuran-3-yl)-4-(4,4,5,5-tetramethyl-1,3,2-dioxaborolan-2-yl)-1H-pyrazole. 1H NMR (CDCl₃, 400 MHz) δ 8.16 (s, 1H), 7.86 (s, 1H), 7.79 (s, 1H), 6.88 (s, 1H), 5.15-5.05 (m, 1H), 4.55-4.45 (m, 1H), 4.25-4.15 (m, 2H), 4.15-4.05 (m, 1H), 3.98 (q, 1H), 3.80-3.70 (m, 1H), 3.45-3.35 (m, 1H), 3.15-3.00 (m, 1H), 2.70-2.60 (m, 1H), 2.60-2.45 (m, 2H), 2.45-2.35 (m, 1H), 2.00-1.90 (m, 1H), 1.75-1.65 (m, 1H), 1.46 (s, 9H), 1.46-1.30 (m, 1H)

### Preparation A3-72: tert-butyl N-[(3S)-1-[2-chloro-5-[1-[(3R)-tetrahydrofuran-3-yl]pyrazol-4-yl]-4-pyridyl]-3-piperidyl]carbamate

The titled compound was prepared in accordance with the same procedures as in Preparation A3-1, changing the boronic acid reagent to (R)-1-(tetrahydrofuran-3-yl)-4-(4,4,5,5-tetramethyl-1,3,2-dioxaborolan-2-yl)-1H-pyrazole. 1H NMR (CDCl₃, 400 MHz) δ 8.15 (s, 1H), 7.85 (s, 1H), 7.78 (s, 1H), 6.87 (s, 1H), 5.10-5.00 (m, 1H), 4.60-4.50 (m, 1H), 4.25-4.15 (m, 2H), 4.15-4.00 (m, 1H), 3.97 (q, 1H), 3.80-3.70 (m, 1H), 3.45-3.35 (m, 1H), 3.15-3.00 (m, 1H), 2.70-2.60 (m, 1H), 2.60-2.45 (m, 2H), 2.45-2.35 (m, 1H), 2.00-1.90 (m, 1H), 1.75-1.65 (m, 1H), 1.46 (s, 9H), 1.46-1.35 (m, 1H)

### Preparation A3-73: tert-butyl (S)-(1-(2-chloro-5-(1-ethyl-1H-pyrazol-4-yl)pyridin-4-yl)piperidin-3-yl)carbamate

The titled compound was prepared in accordance with the same procedures as in Preparation A3-1, changing the boronic acid reagent to 1-ethyl-4-(4,4,5,5-tetramethyl-1,3,2-dioxaborolan-2-yl)-1H-pyrazole. 1H NMR (CDCl₃, 400 MHz) δ 8.19 (s, 1H), 7.81-7.68 (m, 2H), 6.91 (s, 1H), 4.67-4.42 (m, 1H), 4.37 (q, 2H), 3.87-3.64 (m, 1H), 3.52-3.26 (m, 1H), 3.15-2.99 (m, 1H), 2.75-2.45 (m, 2H), 2.05-1.89 (m, 1H), 1.78-1.65 (m, 1H), 1.62 (t, 3H), 1.53 (s, 9H), 1.41-1.32 (m, 1H)

### Preparation A3-74: tert-butyl (S)-(1-(2-chloro-5-(1-isopropyl-1H-pyrazol-4-yl)pyridin-4-yl)piperidin-3-yl)carbamate

The titled compound was prepared in accordance with the same procedures as in Preparation A3-1, changing the boronic acid reagent to 1-isopropylpyrazol-4-boronic acid. 1H NMR (CDCl₃, 400 MHz) δ 8.23 (s, 1H), 7.84-7.67 (m, 2H), 6.91 (s, 1H), 4.65-4.46 (m, 1H), 3.86-3.62 (m, 1H), 3.48-3.32 (m, 1H), 3.14-2.99 (m, 1H), 2.71-2.47 (m, 2H), 2.02-1.88 (m, 1H), 1.74-1.65 (m, 1H), 1.61 (s, 6H), 1.51 (s, 9H), 1.41-1.34 (m, 1H)

### Preparation A3-75: tert-butyl (S)-(1-(2-chloro-5-(1-cyclopropyl-1H-pyrazol-4-yl)pyridin-4-yl)piperidin-3-yl)carbamate

The titled compound was prepared in accordance with the same procedures as in Preparation A3-1, changing the boronic acid reagent to 1-cyclopropyl-4-(4,4,5,5-tetramethyl-1,3,2-dioxaborolan-2-yl)-1H-pyrazole. 1H NMR (CDCl₃, 400 MHz) δ 8.18 (s, 1H), 7.83 (s, 1H), 7.76 (s, 1H), 6.90 (s, 1H), 4.62-4.43 (m, 1H), 3.84-3.63 (m, 2H), 3.49-3.37 (m, 1H), 3.14-3.01 (m, 1H), 2.70-2.45 (m, 2H), 2.04-1.89 (m, 1H), 1.76-1.65 (m, 1H), 1.59-1.52 (m, 1H), 1.52 (s, 9H), 1.41-1.33 (m, 1H), 1.33-1.25 (m, 2H), 1.24-1.21 (m, 1H), 1.14-1.01 (m, 2H)

### Preparation A3-76: tert-butyl (S)-(1-(2-chloro-5-(1-(cyclopropylmethyl)-1H-pyrazol-4-yl)pyridin-4-yl)piperidin-3-yl)carbamate

The titled compound was prepared in accordance with the same procedures as in Preparation A3-1, changing the boronic acid reagent to 1-cyclopropylmethyl-1H-pyrazol-4-boronic acid pinacol ester. 1H NMR (CDCl₃, 400 MHz) δ 8.19 (s, 1H), 7.91 (s, 1H), 7.79 (s, 1H), 6.89 (s, 1H), 4.66 (s, 1H), 4.10 (d, 2H), 3.85-3.68 (m, 1H), 3.46-3.31 (m, 1H), 3.11-2.99 (m, 1H), 2.72-2.50 (m, 2H), 1.98-1.89 (m, 1H), 1.78-1.64 (m, 1H), 1.64-1.50 (m, 1H), 1.42-1.33 (m, 2H), 1.33 (s, 9H), 0.76-0.66 (m, 2H), 0.50-0.35 (m, 2H)

### Preparation A4-1: (S)-1-(6-chloro-6′-(tetrahydro-2H-pyran-4-yl)-[3,3′-bipyridin]-4-yl)piperidin-3-ol

### Step 1: (S)-1-(2-chloro-5-(4,4,5,5-tetramethyl-1,3,2-dioxaborolan-2-yl)pyridin-4-yl)piperidin-3-ol (A4)

2-Chloro-4-fluoro-5-(4,4,5,5-tetramethyl-1,3,2-dioxaborolan-2-yl)pyridine (3.0 g, 11.65 mmol) was dissolved in anhydrous isopropyl alcohol (58.2 mL) and then (S)-3-hydroxypiperidine hydrochloride (1.8 g, 12.82 mmol) and diisopropylethylamine (4.8 mL, 34.95 mmol) were added dropwise thereto. The reaction mixture was stirred at 50 °C for 7 hours. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated to prepare the titled compound, which was used in the subsequent step without further purification.

### Step 2: (S)-1-(6-chloro-6′-(tetrahydro-2H-pyran-4-yl)-[3,3′-bipyridin]-4-yl)piperidin-3-ol (A4-1)

(S)-1-(2-Chloro-5-(4,4,5,5-tetramethyl-1,3,2-dioxaborolan-2-yl)pyridin-4-yl)piperidin-3-ol (100.0 mg, 0.30 mmol) was dissolved in anhydrous 1,4-dioxane(2.0 mL) and then 5-bromo-2-tetrahydropyran-4-yl-pyridine (85.8 mg, 0.35 mmol), [1,1′-bis(diphenylphosphino)ferrocene]dichloropalladium(II)-dichloromethane complex (24.1 mg, 0.03 mmol), and 2.0M sodium carbonate solution (0.71 mL, 1.43 mmol) were added dropwise thereto. Then, the mixed solution was stirred at 80 °C for 4 hours. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (20.1 mg). (Yield: 18.2%) 1H NMR (CDCl₃, 400 MHz) δ 8.69 (s, 1H), 8.03 (s, 1H), 7.78 (d, 1H), 7.28 (d, 1H), 6.90 (s, 1H), 4.14-4.11 (m, 2H), 3.61-3.60 (m, 1H), 3.59-3.55 (m, 2H), 3.21-3.20 (m, 1H), 3.17-3.04 (m, 2H), 2.72-2.67 (m, 2H), 1.96-1.91 (m, 5H), 1.66-1.64 (m, 1H), 1.46-1.44 (m, 2H), 1.42-1.41 (m, 1H)

### Preparation A4-2: (S)-1-(6-chloro-6′-((4-methylpiperazin-1-yl)methyl)-[3,3′-bipyridin]-4-yl)piperidin-3-ol

The titled compound was prepared in accordance with the same procedures as in Preparation A4-1, changing the bromine reagent to 1-((5-bromopyridin-2-yl)methyl)-4-methylpiperazine. 1H NMR (CDCl₃, 400 MHz) δ 8.73 (s, 1H), 8.05 (s, 1H), 7.79-7.77 (m, 1H), 7.46 (d, 1H), 6.90 (s, 1H), 3.81 (s, 2H), 3.65-3.57 (m, 1H), 3.06-3.02 (m, 2H), 2.75-2.71 (m, 1H), 2.61-2.51 (m, 9H), 2.32-2.28 (m, 4H), 1.89-1.83 (m, 2H), 1.52-1.35 (m, 2H)

### Preparation A4-3: (S)-1-(6-chloro-6′-(morpholinomethyl)-[3,3′-bipyridin]-4-yl)piperidin-3-ol

The titled compound was prepared in accordance with the same procedures as in Preparation A4-1, changing the bromine reagent to 4-((5-bromopyridin-2-yl)methyl)morpholine. 1H NMR (CDCl₃, 400 MHz) δ 8.73 (s, 1H), 8.04 (s, 1H), 7.81 (d, 1H), 7.53-7.51 (m, 1H), 6.91 (s, 1H), 3.78-3.76 (m, 4H), 3.73-3.72 (m, 3H), 3.20-3.17 (m, 1H), 2.98-2.93 (m, 1H), 2.76-2.66 (m, 2H), 2.67-2.53 (m, 4H), 1.88-1.86 (m, 1H), 1.67-1.63 (m, 1H), 1.49-1.41 (m, 2H)

### Preparation A4-4: (S)-1-(6-chloro-6′-((4-hydroxypiperidin-1-yl)methyl)-[3,3′-bipyridin]-4-yl)piperidin-3-ol

The titled compound was prepared in accordance with the same procedures as in Preparation A4-1, changing the bromine reagent to 1-[(5-bromo-2-pyridyl)methyl]piperidin-4-ol. 1H NMR (CDCl₃, 400 MHz) δ 8.71 (s, 1H), 8.05 (s, 1H), 7.80 (d, 1H), 7.53-7.51 (m, 1H), 6.91 (s, 1H), 3.84-3.66 (m, 4H), 3.18-3.16 (m, 1H), 3.00-2.97 (m, 1H), 2.82-2.63 (m, 4H), 2.35-2.26 (m, 2H), 1.93-1.86 (m, 3H), 1.76-1.65 (m, 3H), 1.47-1.43 (m, 3H)

### Preparation A4-5: (S)-(6′-chloro-4′-(3-hydroxypiperidin-1-yl)-[3,3′-bipyridin]-6-yl) (4-methylpiperazin-1-yl)methanone

The titled compound was prepared in accordance with the same procedures as in Preparation A4-1, changing the bromine reagent to (5-bromopyridin-2-yl)(4-methylpiperazin-1-yl)methanone. 1H NMR (CDCl₃, 400 MHz) δ 8.76 (s, 1H), 8.38 (s, 1H), 8.05-7.95 (m, 2H), 6.93 (s, 1H), 3.87-3.86 (m, 2H), 3.69-3.68 (m, 2H), 3.63-3.61 (m, 1H), 3.19-3.16 (m, 1H), 3.04-2.99 (m, 1H), 2.70-2.65 (m, 2H), 2.56-2.54 (m, 2H), 2.49-2.46 (m, 2H), 2.35 (s, 3H), 1.90-1.81 (m, 2H), 1.73-1.70 (m, 1H), 1.45-1.42 (m, 2H)

### Preparation A4-6: (S)-(6′-chloro-4′-(3-hydroxypiperidin-1-yl)-[3,3′-bipyridin]-6-yl) (morpholino)methanone

The titled compound was prepared in accordance with the same procedures as in Preparation A4-1, changing the bromine reagent to (5-bromopyridin-2-yl)(morpholino)methanone. 1H NMR (CDCl₃, 400 MHz) δ 8.75 (s, 1H), 8.05 (s, 1H), 8.00-7.97 (m, 1H), 7.86-7.81 (m, 1H), 6.94 (s, 1H), 3.85 (s, 4H), 3.74 (s, 4H), 3.19-3.16 (m, 1H), 3.03-2.96 (m, 1H), 2.80-2.69 (m, 2H), 1.92-1.85 (m, 1H), 1.74-1.68 (m, 2H), 1.47-1.43 (m, 2H)

### Preparation A4-7: (S)-(6′-chloro-4′-(3-hydroxypiperidin-1-yl)-[3,3′-bipyridin]-6-yl) (4-hydroxypiperidin-1-yl)methanone

The titled compound was prepared in accordance with the same procedures as in Preparation A4-1, changing the bromine reagent to (5-bromopyridin-2-yl)(4-hydroxypiperidin-1-yl)methanone. 1H NMR (CDCl₃, 400 MHz) δ 8.76 (s, 1H), 8.06 (s, 1H), 7.96 (d, 1H), 7.54 (d, 1H), 6.93 (s, 1H), 4.26-4.23 (m, 1H), 4.04-4.03 (m, 1H), 3.91-3.87 (m, 1H), 3.75-3.67 (m, 1H), 3.51-3.46 (m, 1H), 3.40-3.35 (m, 1H), 3.19-3.16 (m, 1H), 3.02-2.98 (m, 1H), 2.78-2.65 (m, 2H), 2.08-2.03 (m, 1H), 2.01-1.89 (m, 3H), 1.71-1.64 (m, 2H), 1.47-1.44 (m, 2H)

### Preparation A4-8: (S)-6′-chloro-4′-(3-hydroxypiperidin-1-yl)-[3,3′-bipyridin]-6-carbaldehyde

The titled compound was prepared in accordance with the same procedures as in Preparation A4-1, changing the bromine reagent to 5-bromo-2-pyridinecarboxaldehyde. 1H NMR (CDCl₃, 400 MHz) δ 10.13 (s, 1H), 8.97 (s, 1H), 8.07 (d, 3H), 6.96 (s, 1H), 3.77-3.76 (m, 1H), 3.23-3.19 (m, 1H), 2.99-2.96 (m, 1H), 2.78-2.71 (m, 2H), 1.95-1.92 (m, 1H), 1.71-1.68 (m, 1H), 1.52-1.50 (m, 1H), 1.47-1.43 (m, 2H)

### Preparation A4-9: (S)-1-(6′-chloro-5-((1-methylpiperidin-4-yl)oxy)-[2,3′-bipyridin]-4′-yl)piperidin-3-ol

The titled compound was prepared in accordance with the same procedures as in Preparation A4-1, changing the bromine reagent to 2-bromo-5-[(1-methyl-4-piperidyl)oxy]pyridine. 1H NMR (CD₃OD, 400 MHz) δ 8.43 (s, 1H), 8.08 (s, 1H), 7.75 (d, 1H), 7.62 (d, 1H), 7.05 (s, 1H), 3.70-3.60 (m, 2H), 3.55-3.40 (m, 3H), 3.30-3.20 (m, 1H), 3.15-3.05 (m, 1H), 2.93 (s, 3H), 2.74 (t, 1H), 2.61 (t, 1H), 2.35-2.10 (m, 3H), 2.10-2.00 (m, 1H), 2.00-1.90 (m, 1H), 1.80-1.65 (m, 1H), 1.50-1.25 (m, 3H)

### Preparation A5-1: tert-butyl (S)-(1-(6-chloro-6′-(tetrahydro-2H-pyran-4-yl)-[3,3′-bipyridin]-4-yl)piperidin-3-yl)carbamate

### Step 1: tert-butyl (S)-(1-(2-chloro-5-(4,4,5,5-tetramethyl-1,3,2-dioxaborolan-2-yl)pyridin-4-yl)piperidin-3-yl)carbamate

2-Chloro-4-fluoro-5-(4,4,5,5-tetramethyl-1,3,2-dioxaborolan-2-yl)pyridine (2.0 g, 7.77 mmol) was dissolved in anhydrous isopropyl alcohol (38.8 mL) and then (S)-3-(tert-butoxycarbonylamino)piperidine (2.33 g, 11.65 mmol) and diisopropylethylamine (3.2 mL, 23.30 mmol) were added dropwise thereto. The reaction mixture was stirred at 50 °C for 8 hours. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (1.0 g). (Yield: 29.4%) 1H NMR (CDCl₃, 400 MHz) δ 8.48 (s, 1H), 6.73 (s, 1H), 5.37-5.36 (m, 1H), 3.92-3.89 (m, 1H), 3.19-3.12 (m, 3H), 3.04-3.01 (m, 1H), 1.96-1.95 (m, 1H), 1.82-1.79 (m, 1H), 1.72-1.68 (m, 2H), 1.46 (s, 6H), 1.38 (s, 6H)

### Step 2: tert-butyl (S)-(1-(6-chloro-6′-(tetrahydro-2H-pyran-4-yl)-[3,3′-bipyridin]-4-yl)piperidin-3-yl)carbamate (A5-1)

tert-Butyl (S)-(1-(2-chloro-5-(4,4,5,5-tetramethyl-1,3,2-dioxaborolan-2-yl)pyridin-4-yl)piperidin-3-yl)carbamate (150.0 mg, 0.34 mmol) was dissolved in anhydrous 1,4-dioxane (3.40 mL) and then 5-bromo-2-tetrahydropyran-4-yl-pyridine (99.5 mg, 0.41 mmol), [1,1′-bis(diphenylphosphino)ferrocene]dichloropalladium(II)-dichloromethane complex (27.9 mg, 0.034 mmol), and 2.0M sodium carbonate solution (0.69 mL, 1.37 mmol) were added dropwise thereto. Then, the mixed solution was stirred at 80 °C for 4 hours. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (51.0 mg). (Yield: 31.5%) 1H NMR (CDCl₃, 400 MHz) δ 8.65 (s, 1H), 8.03 (s, 1H), 7.82 (d, 1H), 7.28 (s, 1H), 6.89 (s, 1H), 4.51-4.41 (m, 1H), 4.14-4.11 (m, 2H), 3.61-3.55 (m, 3H), 3.37-3.34 (m, 1H), 3.04-2.98 (m, 2H), 2.64-2.54 (m, 2H), 1.94-1.82 (m, 4H), 1.54-1.53 (m, 1H), 1.43 (s, 9H), 1.29-1.27 (m, 1H)

### Preparation A5-2: tert-butyl N-[(3S)-1-[2-chloro-5-[6-(1-methyl-4-piperidyl)-3-pyridyl]-4-pyridyl]-3-piperidyl]carbamate

The titled compound was prepared in accordance with the same procedures as in Preparation A5-1, changing the bromine reagent to 5-bromo-2-(1-methyl-4-piperidyl)pyridine. 1H NMR (CDCl₃, 400 MHz) δ 8.63 (s, 1H), 8.02 (s, 1H), 7.79 (d, 1H), 7.29 (d, 1H), 6.88 (s, 1H), 4.52-4.44 (m, 1H), 3.72-3.65 (m, 1H), 3.37-3.34 (m, 1H), 3.04-2.94 (m, 4H), 2.79-2.72 (m, 2H), 2.63-2.55 (m, 2H), 2.35 (s, 3H), 2.14-2.12 (m, 2H), 2.09-2.05 (m, 2H), 2.01-1.87 (m, 4H), 1.45 (s, 9H)

### Preparation A5-3: tert-butyl (S)-(1-(2-chloro-5-(2-(1-methylpiperidin-4-yl)thiazol-5-yl)pyridin-4-yl)piperidin-3-yl)carbamate

The titled compound was prepared in accordance with the same procedures as in Preparation A5-1, changing the bromine reagent to 5-bromo-2-(1-methyl-4-piperidinyl)thiazole. 1H NMR (CD₃OD, 400 MHz) δ 8.46 (s, 1H), 8.18 (s, 1H), 7.93 (s, 1H), 3.89-3.56 (m, 3H), 3.44 (t, 1H), 3.35 (d, 1H), 3.20-3.14 (m, 3H), 2.88 (s, 3H), 2.71 (t, 1H), 2.41 (d, 3H), 2.24-2.17 (m, 2H), 1.93 (d, 1H), 1.78 (brs, 1H), 1.70-1.66 (m, 1H), 1.40 (s, 9H), 1.39-1.25 (m, 1H)

### Preparation A6-1: (S)-1-(2-chloro-5-((1-(trifluoromethyl)-1H-pyrazol-4-yl)ethynyl)pyridin-4-yl)piperidin-3-ol

(S)-1-(2-Chloro-5-iodopyridin-4-yl)piperidin-3-ol (A2, 500.0 mg, 1.48 mmol) was dissolved in anhydrous 1,4-dioxane (10.0 mL) and then 4-ethynyl-1-(trifluoromethyl)-1H-pyrazole (354.6 mg, 2.21 mmol), bis(triphenylphosphine)palladium(II) dichloride (51.8 mg, 0.074 mmol), copper(I) iodide (16.9 mg, 0.089 mmol), and triethylamine (0.41 mL, 2.95 mmol) were added dropwise thereto. Then, the mixed solution was stirred at 80 °C for 4 hours. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (491.7 mg). (Yield: 89.8%) 1H NMR (CDCl₃, 400 MHz) δ 8.30 (s, 1H), 8.11 (s, 1H), 7.93 (s, 1H), 6.80 (s, 1H), 4.02-4.01 (m, 1H), 3.55-3.51 (m, 2H), 3.36-3.34 (m, 2H), 2.12-1.89 (m, 2H), 1.77-1.72 (m, 2H)

### Preparation A6-2: (S)-1-(2-chloro-5-((tetrahydro-2H-pyran-4-yl)ethynyl)pyridin-4-yl)piperidin-3-ol

The titled compound was prepared in accordance with the same procedures as in Preparation A6-1, changing the ethyne reagent to 4-ethynyl-tetrahydro-2H-pyrane. 1H NMR (CDCl₃, 400 MHz) δ 8.19 (s, 1H), 6.74 (s, 1H), 3.99-3.94 (m, 3H), 3.61-3.52 (m, 3H), 3.40-3.37 (m, 1H), 3.33-3.28 (m, 1H), 3.24-3.22 (m, 1H), 3.80 (s, 1H), 2.20-2.19 (m, 1H), 1.98-1.92 (m, 4H), 1.85-1.82 (m, 2H)

### Preparation A6-3: (S)-1-(2-chloro-5-((1-(difluoromethyl)-1H-pyrazol-4-yl)ethynyl)pyridin-4-yl)piperidin-3-ol

The titled compound was prepared in accordance with the same procedures as in Preparation A6-1, changing the ethyne reagent to 1-(difluoromethyl)-4-ethynyl-1H-pyrazole. 1H NMR (CDCl₃, 400 MHz) δ 8.33 (s, 1H), 8.13 (s, 1H), 7.88 (s, 1H), 7.38-7.35 (m, 0.2H), 7.23-7.19 (m, 0.6H), 7.08-7.04 (m, 0.2H), 6.84 (s, 1H), 4.07-3.97 (m, 1H), 3.50-3.44 (m, 1H), 3.43-3.33 (m, 1H), 3.33-3.26 (m, 1H), 2.23-2.12 (m, 1H), 2.06-1.85 (m, 1H), 1.83-1.69 (m, 2H)

### Preparation A6-4: (S)-1-(2-chloro-5-((1-(2,2-difluoroethyl)-1H-pyrazol-4-yl)ethynyl)pyridin-4-yl)piperidin-3-ol

The titled compound was prepared in accordance with the same procedures as in Preparation A6-1, changing the ethyne reagent to 1-(2,2-difluoroethyl)-4-ethynyl-1H-pyrazole. 1H NMR (CDCl₃, 400 MHz) δ 8.33-8.34 (s, 1H), 7.81 (s, 2H), 6.83 (s, 1H), 6.30-6.24 (m, 0.2H), 6.16-6.10 (m, 0.6H), 6.01-5.97 (m, 0.2H), 4.56-4.44 (m, 2H), 4.07-3.97 (m, 1H), 3.59-3.47 (m, 2H), 3.41-3.27 (m, 2H), 2.07-1.95 (m, 1H), 1.95-1.86 (m, 1H), 1.80-1.70 (m, 2H)

### Preparation A6-5: (S)-1-(2-chloro-5-((1-(2,2,2-trifluoroethyl)-1H-pyrazol-4-yl)ethynyl)pyridin-4-yl)piperidin-3-ol

The titled compound was prepared in accordance with the same procedures as in Preparation A6-1, changing the ethyne reagent to 4-ethynyl-1-(2,2,2-trifluoroethyl)pyrazole. 1H NMR (CDCl₃, 400 MHz) δ 8.33 (s, 1H), 7.84 (s, 1H), 7.81 (s, 1H), 6.82 (s, 1H), 4.07-3.97 (m, 1H), 3.59-3.46 (m, 2H), 3.40-3.25 (m, 2H), 2.19-2.14 (m, 1H), 1.95-1.84 (m, 1H), 1.81-1.69 (m, 2H)

### Preparation A7-1: tert-butyl (S)-(1-(2-chloro-5-((tetrahydro-2H-pyran-4-yl)ethynyl)pyridin-4-yl)piperidin-3-yl)carbamate

tert-Butyl (S)-(1-(2-chloro-5-iodopyridin-4-yl)piperidin-3-yl)carbamate(300.0 mg, 0.68 mmol) was dissolved in anhydrous 1,4-dioxane (8.0 mL) and then 4-ethynyl-tetrahydro-2H-pyrane (150.9 mg, 1.37 mmol), bis(triphenylphosphine)palladium(II) dichloride (22.9 mg, 0.033 mmol), copper(I) iodide (7.8 mg, 0.041 mmol), and triethylamine (0.19 mL, 1.37 mmol) were added dropwise thereto. Then, the mixed solution was stirred at 80 °C for 4 hours. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (120.4 mg). (Yield: 41.8%) 1H NMR (CDCl₃, 400 MHz) δ 8.19 (s, 1H), 7.27 (s, 1H), 4.89-4.88 (m, 1H), 3.97-3.95 (m, 2H), 3.93-3.91 (m, 1H), 3.57-3.52 (m, 3H), 3.39-3.37 (m, 1H), 3.20-3.19 (m, 2H), 2.96-2.92 (m, 1H), 1.94-1.92 (m, 2H), 1.86-1.63 (m, 6H), 1.43 (s, 9H)

### Preparation A7-2: tert-butyl (S)-(1-(2-chloro-5-((1-(trifluoromethyl)-1H-pyrazol-4-yl)ethynyl)pyridin-4-yl)piperidin-3-yl)carbamate

The titled compound was prepared in accordance with the same procedures as in Preparation A7-1, changing the ethyne reagent to 4-ethynyl-1-(trifluoromethyl)-1H-pyrazole. 1H NMR (CDCl₃, 400 MHz) δ 8.29-8.28 (m, 2H), 7.92 (s, 1H), 6.79 (s, 1H), 4.74-4.73 (m, 1H), 3.89-3.74 (m, 2H), 3.39-3.21 (m, 1H), 3.08-2.99 (m, 1H), 1.92-1.87 (m, 2H), 1.77-1.75 (m, 1H), 1.58-1.55 (m, 1H), 1.42 (s, 9H)

### Preparation A7-3: tert-butyl (S)-(1-(2-chloro-5-((1-(difluoromethyl)-1H-pyrazol-4-yl)ethynyl)pyridin-4-yl)piperidin-3-yl)carbamate

The titled compound was prepared in accordance with the same procedures as in Preparation A7-1, changing the ethyne reagent to 1-(difluoromethyl)-4-ethynyl-1H-pyrazole. 1H NMR (CDCl₃, 400 MHz) δ 8.28 (s, 1H), 8.19 (s, 1H), 7.82 (s, 1H), 7.33-7.03 (m, 1H), 6.77 (s, 1H), 4.80-4.78 (m, 1H), 3.88 (brs, 1H), 3.80-3.77 (m, 1H), 3.41-3.38 (m, 1H), 3.12-3.11 (m, 2H), 1.92-1.88 (m, 2H), 1.76-1.75 (m, 1H), 1.74-1.73 (m, 1H), 1.42 (s, 9H)

### Preparation A7-4: tert-butyl (S)-(1-(2-chloro-5-((1-(2,2-difluoroethyl)-1H-pyrazol-4-yl)ethynyl)pyridin-4-yl)piperidin-3-yl)carbamate

The titled compound was prepared in accordance with the same procedures as in Preparation A7-1, changing the ethyne reagent to 1-(2,2-difluoroethyl)-4-ethynyl-1H-pyrazole. 1H NMR (CDCl₃, 400 MHz) δ 8.26 (s, 1H), 7.81 (s, 1H), 7.74 (s, 1H), 6.76 (s, 1H), 6.25-5.95 (m, 1H), 4.82-4.80 (m, 1H), 4.50-4.42 (m, 2H), 3.87-3.86 (m, 1H), 3.72-3.69 (m, 1H), 3.43-3.40 (m, 1H), 3.17-3.15 (m, 2H), 1.92-1.88 (m, 2H), 1.75-1.74 (m, 1H), 1.73-1.71 (m, 1H), 1.43 (s, 9H)

### Preparation A7-5: tert-butyl (S)-(1-(2-chloro-5-((1-(2,2,2-trifluoroethyl)-1H-pyrazol-4-yl)ethynyl)pyridin-4-yl)piperidin-3-yl)carbamate

The titled compound was prepared in accordance with the same procedures as in Preparation A7-1, changing the ethyne reagent to 4-ethynyl-1-(2,2,2-trifluoroethyl)pyrazole. 1H NMR (CDCl₃, 400 MHz) δ 8.27 (s, 1H), 7.86 (s, 1H), 7.76 (s, 1H), 6.76 (s, 1H), 4.80-4.78 (m, 1H), 4.73-4.67 (q, 2H), 3.87-3.86 (m, 1H), 3.75-3.72 (m, 1H), 3.44-3.41 (m, 1H), 3.16-3.11 (m, 2H), 1.92-1.88 (m, 2H), 1.76-1.74 (m, 1H), 1.72-1.70 (m, 1H), 1.42 (s, 9H)

### Preparation A7-6: tert-butyl (S)-(1-(2-chloro-5-((4-methyltetrahydro-2H-pyran-4-yl)ethynyl)pyridin-4-yl)piperidin-3-yl)carbamate

The titled compound was prepared in accordance with the same procedures as in Preparation A7-1, changing the ethyne reagent to 4-ethynyl-4-methyltetrahydro-2H-pyrane. 1H NMR (CDCl₃, 400 MHz) δ 8.66 (s, 1H), 6.86 (s, 1H), 5.03-4.84 (m, 1H), 3.96 (s, 1H), 3.88-3.82 (m, 3H), 3.24-3.19 (m, 1H), 3.10-2.94 (m, 2H), 2.88-2.78 (m, 1H), 2.10-2.02 (m, 2H), 1.38 (s, 9H)

### Preparation A7-7: tert-butyl (S)-(1-(2-chloro-5-((4-hydroxytetrahydro-2H-pyran-4-yl)ethynyl)pyridin-4-yl)piperidin-3-yl)carbamate

The titled compound was prepared in accordance with the same procedures as in Preparation A7-1, changing the ethyne reagent to 4-ethynyltetrahydro-2H-pyran-4-ol. 1H NMR (CDCl₃, 400 MHz) δ 8.29 (s, 1H), 6.80 (s, 1H), 4.65-4.50 (m, 2H), 4.03-3.92 (m, 2H), 3.90-3.80 (m, 1H), 3.80-3.70 (m, 2H), 3.40-3.29 (m, 1H), 2.86-2.73 (m, 1H), 2.37-2.26 (m, 1H), 2.15-2.09 (m, 2H), 2.03-1.93 (m, 3H), 1.87-1.75 (m, 1H), 1.55 (s, 9H)

### Preparation A7-8: tert-butyl (S)-(1-(2-chloro-5-(3-hydroxy-3-methylbut-1-yn-1-yl)pyridin-4-yl)piperidin-3-yl)carbamate

The titled compound was prepared in accordance with the same procedures as in Preparation A7-1, changing the ethyne reagent to 2-methylbut-3-yn-2-ol. 1H NMR (CDCl₃, 400 MHz) δ 8.26 (s, 1H), 6.80 (s, 1H), 4.64-4.53 (m, 1H), 4.53-4.42 (m, 1H), 3.98-3.78 (m, 1H), 3.36-3.25 (m, 1H), 2.91-2.74 (m, 1H), 2.47-2.32 (m, 1H), 2.11-2.00 (m, 1H), 2.00-1.87 (m, 1H), 1.87-1.78 (m, 1H), 1.70 (s, 6H), 1.52 (s, 9H)

### Preparation A7-9: tert-butyl (S)-(1-(2-chloro-5-(4-hydroxybut-1-yn-1-yl)pyridin-4-yl)piperidin-3-yl)carbamate

The titled compound was prepared in accordance with the same procedures as in Preparation A7-1, changing the ethyne reagent to 3-butyn-1-ol. 1H NMR (CDCl₃, 400 MHz) δ 8.27 (s, 1H), 6.80 (s, 1H), 4.90-4.80 (m, 1H), 4.20 (q, 2H), 3.82-3.74 (m, 1H), 3.35-3.22 (m, 1H), 3.12-3.04 (m, 2H), 1.99-1.85 (m, 2H), 1.82-1.71 (m, 1H), 1.54 (s, 9H), 1.33 (t, 2H)

### Preparation A8-1: (S)-1-(6-chloro-6′-(((S)-3-fluoropyrrolidin-1-yl)methyl)-[3,3′-bipyridin]-4-yl)piperidin-3-ol

(S)-6′-chloro-4′-(3-hydroxypiperidin-1-yl)-[3,3′-bipyridin]-6-carbaldehyde(A4-8, 40 mg, 0.13 mmol) was dissolved in dichloromethane (1.0 mL) and then (S)-(+)-3-fluoropyrrolidine hydrochloride (13.0 mg, 0.10 mmol) and triethylamine (0.03 ml, 0.19 mmol) were added dropwise thereto. Then, the mixed solution was stirred at room temperature for 10 minutes. The reaction mixture was cooled to 0 °C with ice water and then sodium borohydride (60.0 mg, 0.28 mmol) was added dropwise thereto. The reaction mixture was stirred at room temperature for 2 hours. After completion of the reaction, dichloromethane was added to the reaction mixture and then a saturated sodium hydrogen carbonate solution was added thereto. The extracted organic layer was dried over anhydrous magnesium sulfate and then filtered. The filtrate was concentrated to prepare the titled compound (22.5 mg), without further purification. (Yield: 61.0%) 1H NMR (CDCl₃, 400 MHz) δ 8.67 (s, 1H), 8.03-7.99 (m, 2H), 7.64 (d, 1H), 7.06 (s, 1H), 3.87 (s, 2H), 3.60-3.57 (m, 1H), 3.28-3.25 (m, 2H), 3.12-3.08 (m, 1H), 3.02-2.92 (m, 2H), 2.79-2.71 (m, 1H), 2.59-2.53 (m, 2H), 2.25-2.10 (m, 1H), 2.07-1.91 (m, 2H), 1.70-1.66 (m, 1H), 1.44-1.25 (m, 3H)

### Preparation A8-2: (3S)-1-[2-chloro-5-[6-[(3-hydroxypyrrolidin-1-yl)methyl]-3-pyridyl]-4-pyridyl]piperidin-3-ol

The titled compound was prepared in accordance with the same procedures as in Preparation A8-1, changing the amine reagent to pyrrolidin-3-ol. 1H NMR (CDCl₃, 400 MHz) δ 8.70 (s, 1H), 8.02-7.99 (m, 2H), 7.64 (d, 1H), 7.07 (s, 1H), 4.41 (brs, 1H), 4.01-3.99 (m, 2H), 3.60-3.56 (m, 1H), 3.28-3.25 (m, 1H), 3.12-3.09 (m, 1H), 2.99-2.98 (m, 2H), 2.79-2.69 (m, 3H), 2.57 (t, 1H), 2.23-2.16 (m, 1H), 2.02-1.91 (m, 1H), 1.83-1.80 (m, 1H), 1.70-1.67 (m, 1H), 1.44-1.25 (m, 3H)

### Preparation A8-3: (3S)-1-[2-chloro-5-[6-[[3-(dimethylamino)pyrrolidin-1-yl]methyl]-3-pyridyl]-4-pyridyl]piperidin-3-ol

The titled compound was prepared in accordance with the same procedures as in Preparation A8-1, changing the amine reagent to 3-(dimethylamino)pyrrolidine. 1H NMR (CDCl₃, 400 MHz) δ 8.70 (s, 1H), 8.05 (s, 1H), 7.80-7.75 (m, 1H), 7.46 (d, 1H), 6.90 (s, 1H), 7.38 (d, 1H), 6.89 (d, 1H), 3.98 (s, 2H), 3.55-3.52 (m, 1H), 3.08-3.04 (m, 2H), 2.85-2.78 (m, 6H), 2.49-2.47 (m, 1H), 2.38-2.34 (m, 4H), 2.22 (s, 6H), 2.03-1.99 (m, 1H), 2.22 (s, 1H), 1.79-1.72 (m, 1H)

### Preparation A8-4: 1-((6′-chloro-4′-((S)-3-hydroxypiperidin-1-yl)-[3,3′-bipyridin]-6-yl)methyl)piperidin-3-ol

The titled compound was prepared in accordance with the same procedures as in Preparation A8-1, changing the amine reagent to 3-hydroxypiperidine. 1H NMR (CDCl₃, 400 MHz) δ 8.80-8.70 (m, 1H), 8.05 (s, 1H), 7.84-7.78 (m, 1H), 7.51-7.45 (m, 1H), 6.90 (d, 1H), 3.85-3.82 (m, 2H), 3.61-3.58 (m, 1H), 3.14-3.03 (m, 3H), 2.76-2.71 (m, 2H), 2.56-2.38 (m, 7H), 1.95-1.87 (m, 5H), 1.48-1.39 (m, 2H)

### Preparation A8-5: tert-butyl (S)-(1-((6′-chloro-4′-(3-hydroxypiperidin-1-yl)-[3,3′-bipyridin]-6-yl)methyl)piperidin-4-yl)carbamate

The titled compound was prepared in accordance with the same procedures as in Preparation A8-1, changing the amine reagent to 4-(tert-butoxycarbonylamino)piperidine. 1H NMR (CDCl₃, 400 MHz) δ 8.68 (s, 1H), 8.00 (s, 2H), 7.63 (d, 1H), 7.07 (s, 1H), 3.75 (s, 2H), 3.60-3.58 (m, 1H), 3.60-3.56 (m, 1H), 3.38-3.23 (m, 2H), 3.14-3.08 (m, 1H), 2.94-2.91 (m, 2H), 2.73-2.68 (m, 1H), 2.57 (t, 1H), 2.30-2.20 (m, 2H), 1.94-1.85 (m, 3H), 1.72-1.65 (m, 1H), 1.57-1.52 (m, 2H), 1.44 (s, 9H), 1.36-1.34 (m, 1H)

### Preparation A9-1: (S)-1-(2-chloro-5-(1-(1-methylpiperidin-4-yl)-1H-pyrazol-4-yl)pyrimidin-4-yl)piperidin-3-ol

### Step 1: (S)-1-(2-chloro-5-iodopyrimidin-4-yl)piperidin-3-ol (A9)

2,4-Dichloro-5-iodo-pyrimidine (500 mg, 1.82 mmol) was dissolved in anhydrous acetonitrile (10.0 mL) and the (S)-3-hydroxypiperidine hydrochloride (275.3 mg, 2.00 mmol) and diisopropylethylamine (1.6 mL, 11.65 mmol) were added dropwise thereto. The reaction mixture was stirred 50 °C for 4 hours. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (290 mg). (Yield: 46.9%) 1H NMR (CDCl₃, 400 MHz) δ 8.48 (s, 1H), 3.99 (s, 1H), 3.85 (d, 1H), 3.71/3.67 (m, 1H), 3.62-3.52 (m, 2H), 2.29-2.28 (m, 1H), 1.95-1.93 (m, 2H), 1.74-1.61 (m, 2H)

### Step 2: (S)-1-(2-chloro-5-(1-(1-methylpiperidin-4-yl)-1H-pyrazol-4-yl)pyrimidin-4-yl)piperidin-3-ol (A9-1)

(S)-1-(2-Chloro-5-iodopyrimidin-4-yl)piperidin-3-ol (200 mg, 0.59 mmol) was dissolved in anhydrous 1,4-dioxane (6.0 mL) and then 1-methyl-4-(4-(4,4,5,5-tetramethyl-1,3,2-dioxaborolan-2-yl)-1H-pyrazol-1-yl)piperidine (205.8 mg, 0.71 mmol), [1,1′-bis(diphenylphosphino)ferrocene]dichloropalladium(II)-dichloromethane complex (48.2 mg, 0.06 mmol), and 2.0M sodium carbonate solution (1.48 mL, 2.95 mmol) were added dropwise thereto. Then, the mixed solution was stirred at 80 °C for 6 hours. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (50.0 mg). (Yield: 22.5%) 1H NMR (CDCl₃, 400 MHz) δ 8.03 (s, 1H), 7.60 (s, 1H), 7.59 (s, 1H), 4.19-4.15 (m, 1H), 3.90-3.88 (m, 1H), 3.59-3.58 (m, 1H), 3.56-3.55 (m, 1H), 3.48-3.44 (m, 1H), 3.32-3.29 (m, 1H), 3.22-3.02 (m, 2H), 2.34 (s, 3H), 2.22-2.03 (m, 6H), 1.85-1.82 (m, 1H), 1.78-1.75 (m, 1H), 1.66-1.63 (m, 1H), 1.45-1.44 (m, 1H)

### Preparation A10-1: tert-butyl (S)-(1-(6-chloro-3-(1-(2,2,2-trifluoroethyl)-1H-pyrazol-4-yl)pyridazin-4-yl)piperidin-3-yl) carbamate

### Step 1: tert-butyl (S)-(1-(3-bromo-6-chloropyridazin-4-yl)piperidin-3-yl)carbamate (A10)

3-Bromo-4,6-dichloropyridazine (500.0 mg, 2.19 mmol) was dissolved in anhydrous dimethylacetamide (5.0 mL) and then (S)-3-(tert-butoxycarbonylamino)piperidine (659.1 mg, 3.29 mmol) and diisopropylethylamine (1.15 mL, 6.58 mmol) were added dropwise thereto. The reaction mixture was stirred at 90 °C for 3 hours. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (700.0 mg). (Yield: 81.6%) 1H NMR (CDCl₃, 400 MHz) δ 6.88 (s, 1H), 4.83 (s, 1H), 3.91 (s, 1H), 3.51-3.48 (m, 1H), 3.39-3.34 (m, 1H), 3.14-3.11 (m, 1H), 3.09-3.00 (m, 1H), 1.97-1.90 (m, 2H), 1.89-1.85 (m, 1H), 1.72 (s, 9H), 1.70-1.68 (m, 1H)

### Step 2: tert-butyl (S)-(1-(6-chloro-3-(1-(2,2,2-trifluoroethyl)-1H-pyrazol-4-yl)pyridazin-4-yl)piperidin-3-yl) carbamate (A10-1)

tert-Butyl (S)-(1-(6-chloro-3-bromopyridazin-4-yl)piperidin-3-yl)carbamate (100 mg, 0.25 mmol) was dissolved in anhydrous 1,4-dioxane (3.0 mL) and then 4-(4,4,5,5-tetramethyl-1,3,2-dioxaborolan-2-yl)-1-(2,2,2-trifluoroethyl)pyrazole (70.5 mg, 0.25 mmol), [1,1′-bis(diphenylphosphino)ferrocene]dichloropalladium(II)-dichloromethane complex (10.4 mg, 0.013 mmol), and 2.0M sodium carbonate solution (0.38 mL, 0.76 mmol) were added dropwise thereto. Then, the mixed solution was stirred at 90 °C overnight. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (75.4 mg). (Yield: 64.0%) 1H NMR (CDCl₃, 400 MHz) δ 8.25 (s, 1H), 8.24 (s, 1H), 7.00 (s, 1H), 4.89-4.81 (m, 2H), 4.52-4.51 (m, 1H), 3.80 (s, 1H), 3.56-3.53 (m, 1H), 3.24-3.21 (m, 1H), 2.68-2.67 (m, 1H), 2.50-2.49 (m, 1H), 2.04-2.03 (m, 1H), 1.85-1.82 (m, 1H), 1.81-1.80 (m, 1H), 1.73 (s, 9H), 1.40-1.39 (m, 1H)

### Preparation A11-1: methyl (S)-6-chloro-4-(3-hydroxypiperidin-1-yl)nicotinate

Methyl-4,6-dichloronicotinate (1.0 g, 4.85 mmol) was dissolved in anhydrous dimethylformamide (20.0 mL) and then (S)-3-hydroxypiperidine hydrochloride (734.7 mg, 5.34 mmol) and diisopropylethylamine (1.7 mL, 9.71 mmol) were added dropwise thereto. The reaction mixture was stirred at 50 °C for 4 hours. After completion of the reaction, the reaction mixture was cooled to room temperature. Water was added to the reaction mixture and the aqueous layer was extracted with ethyl acetate. The extracted organic layer was dried over anhydrous magnesium sulfate and then filtered. The filtrate was concentrated and then purified by silica gel column chromatography to prepare the titled compound (1.13 g, Yield: 86.0%). 1H NMR (CDCl₃, 400 MHz) δ 8.60 (s, 1H), 6.86 (s, 1H), 4.00-3.97 (m, 1H), 3.94 (s, 3H), 3.24-3.18 (m, 3H), 3.14-3.09 (m, 1H), 2.90-2.87 (m, 1H), 2.07-2.03 (m, 1H), 1.83-1.75 (m, 2H), 1.70-1.63 (m, 1H)

### Preparation A11-2: (S)-6-chloro-4-(3-hydroxypiperidin-1-yl)nicotinamide

The titled compound was prepared in accordance with the same procedures as in Preparation A11-1, changing the dichloro reagent to 4,6-dichloronicotinamide. 1H NMR (CD₃OD, 400 MHz) δ 8.20 (s, 1H), 7.00 (s, 1H), 3.86-3.82 (m, 1H), 3.54-3.50 (m, 1H), 3.39-3.36 (m, 1H), 3.11-3.05 (m, 1H), 2.97-2.92 (m, 1H), 2.03-1.89 (m, 2H), 1.69-1.65 (m, 1H), 1.57-1.2 (m, 1H)

### Preparation B1-1: tert-butyl (S)-(1-(2-chloro-5-(1-(difluoromethyl)-1H-pyrazol-4-yl)pyridin-4-yl)piperidin-3-yl)(methyl)carbamate

### Step 1: 2-chloro-5-(1-(difluoromethyl)-1H-pyrazol-4-yl)-4-fluoropyridine (B1)

2-Chloro-4-fluoro-5-iodo-pyridine (5.0 g, 19.42 mmol) was dissolved in anhydrous 1,4-dioxane (97.1 mL) and then 1-(difluoromethyl)pyrazol-4-boronic acid pinacol ester (5.7 g, 23.30 mmol), bis(triphenylphosphine)palladium(II) dichloride (1.36 g, 1.94 mmol), and 2.0M sodium carbonate solution (29.1 mL, 58.3 mmol) were added dropwise thereto. Then, the mixed solution was stirred at 80 °C for 2 hours. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (3.7 g). (Yield: 76.9%) 1H NMR (CDCl₃, 400 MHz) δ 8.63 (d, 1H), 8.23 (s, 1H), 8.04 (s, 1H), 7.41-7.11 (m, 2H)

### Step 2: tert-butyl (S)-(1-(2-chloro-5-(1-(difluoromethyl)-1H-pyrazol-4-yl)pyridin-4-yl)piperidin-3-yl)(methyl)carbamate (B1-1)

2-Chloro-5-(1-(difluoromethyl)-1H-pyrazol-4-yl)-4-fluoropyridine (200 mg, 0.81 mmol) was dissolved in anhydrous dimethylformamide (8.0 mL) and then tert-butyl N-methyl-N-[(3S)-3-piperidyl]carbamate (173.1 mg, 0.81 mmol) and diisopropylethylamine (0.33 mL, 2.42 mmol) were added dropwise thereto. The reaction mixture was stirred at 80 °C overnight. Water was added to the reaction mixture and then the aqueous layer was extracted with ethyl acetate. The extracted organic layer was dried over anhydrous magnesium sulfate and then filtered. The filtrate was concentrated and then purified by silica gel column chromatography to prepare the titled compound (208.6 mg). (Yield: 58.4%) 1H NMR (CDCl₃, 400 MHz) δ 8.15 (s, 2H), 7.97 (s, 1H), 7.41-7.11 (m, 1H), 6.91 (s, 1H), 3.28-3.22 (m, 2H), 2.79 (s, 3H), 2.75-2.71 (m, 1H), 2.43-2.31 (m, 1H), 1.89-1.88 (m, 1H), 1.74-1.73 (m, 1H), 1.63-1.59 (m, 2H), 1.48 (s, 9H)

### Preparation B1-2: tert-butyl (R)-(1-(2-chloro-5-(1-(difluoromethyl)-1H-pyrazol-4-yl)pyridin-4-yl)piperidin-3-yl)(methyl)carbamate

The titled compound was prepared in accordance with the same procedures as in Preparation B1-1, changing the amine reagent to tert-butyl N-methyl-N-[(3R)-3-piperidyl]carbamate. 1H NMR (CDCl₃, 400 MHz) δ 8.16 (s, 2H), 7.97 (s, 1H), 7.42-7.11 (m, 1H), 6.91 (s, 1H), 3.29-3.25 (m, 2H), 2.79 (s, 3H), 2.74-2.71 (m, 1H), 2.45-2.43 (m, 1H), 1.90-1.89 (m, 1H), 1.88-1.74 (m, 1H), 1.72-1.64 (m, 2H), 1.48 (s, 9H)

### Preparation B1-3: tert-butyl((3S,4R)-1-(2-chloro-5-(1-(difluoromethyl)-1H-pyrazol-4-yl)pyridin-4-yl)-4-methylpiperidin-3-yl)carbamate

The titled compound was prepared in accordance with the same procedures as in Preparation B1-1, changing the amine reagent to tert-butyl N-[(3S,4R)-4-methyl-3-piperidyl]carbamate. 1H NMR (CDCl₃, 400 MHz) δ 8.15 (s, 1H), 8.12 (s, 1H), 7.96 (s, 1H), 7.27 (t, 1H), 6.91 (s, 1H), 4.36-4.33 (m, 1H), 3.53 (d, 1H), 3.40-3.31 (m, 1H), 3.23 (d, 1H), 2.52 (t, 1H), 2.38 (t, 1H), 1.73-1.70 (m, 1H), 1.46 (s, 9H), 1.40-1.30 (m, 2H), 1.07 (d, 3H)

### Preparation B1-4: tert-butyl ((3S,5S)-1-(2-chloro-5-(1-(difluoromethyl)-1H-pyrazol-4-yl)pyridin-4-yl)-5-methylpiperidin-3-yl)carbamate

The titled compound was prepared in accordance with the same procedures as in Preparation B1-1, changing the amine reagent to tert-butyl N-[(3S,5S)-5-methyl-3-piperidyl]carbamate. 1H NMR (CDCl₃, 400 MHz) δ 8.14 (s, 1H), 8.06 (s, 1H), 7.91 (s, 1H), 7.28 (t, 1H), 6.91 (s, 1H), 7.44 (brs, 1H), 3.97-3.89 (m, 1H), 3.19-3.09 (m, 2H), 2.97-2.93 (m, 1H), 2.27-2.22 (m, 1H), 1.86-1.83 (m, 2H), 1.46 (s, 9H), 1.35-1.27 (m, 1H), 0.82 (d, 3H)

### Preparation B1-5: tert-butyl (S)-(1-(2-chloro-5-(1-(difluoromethyl)-1H-pyrazol-4-yl)pyridin-4-yl)-3-methylpiperidin-3-yl)carbamate

The titled compound was prepared in accordance with the same procedures as in Preparation B1-1, changing the amine reagent to tert-butyl N-[(3S)-3-methyl-3-piperidyl]carbamate. 1H NMR (CDCl₃, 400 MHz) δ 8.19 (s, 1H), 8.16 (s, 1H), 7.94 (s, 1H), 7.27 (t, 1H), 6.90 (s, 1H), 4.59 (brs, 1H), 3.50 (d, 1H), 3.11 (d, 1H), 2.55-2.49 (m, 1H), 2.17-2.14 (m, 1H), 1.69-1.60 (m, 1H), 1.50-1.39 (m, 13H), 1.35-1.31 (m, 2H)

### Preparation B1-6: tert-butyl ((3R,4S)-1-(2-chloro-5-(1-(difluoromethyl)-1H-pyrazol-4-yl)pyridin-4-yl)-4-fluoropiperidin-3-yl)carbamate

The titled compound was prepared in accordance with the same procedures as in Preparation B1-1, changing the amine reagent to tert-butyl N-[(3R,4S)-4-fluoro-3-piperidyl]carbamate. 1H NMR (CDCl₃, 400 MHz) δ 8.17 (s, 1H), 8.13 (s, 1H), 7.95 (s, 1H), 7.27 (t, 1H), 6.95 (s, 1H), 4.95 (brs, 1H), 4.89 (d, 1H), 3.94-3.87 (m, 1H), 3.34-3.30 (m, 1H), 3.10-3.05 (m, 1H), 2.85-2.80 (m, 2H), 2.07-2.01 (m, 1H), 1.90-1.73 (m, 1H), 1.47 (s, 9H)

### Preparation B1-7: tert-butyl ((3R,4R)-1-(2-chloro-5-(1-(difluoromethyl)-1H-pyrazol-4-yl)pyridin-4-yl)-4-fluoropiperidin-3-yl)carbamate

The titled compound was prepared in accordance with the same procedures as in Preparation B1-1, changing the amine reagent to tert-butyl N-[(3R,4R)-4-fluoro-3-piperidyl]carbamate. 1H NMR (CDCl₃, 400 MHz) δ 8.18 (s, 1H), 8.11 (s, 1H), 7.95 (s, 1H), 7.28 (t, 1H), 6.95 (s, 1H), 4.70 (brs, 1H), 4.63-4.47 (m, 1H), 3.90-3.82 (m, 1H), 3.57-3.54 (m, 1H), 3.14-3.07 (m, 1H), 2.80-2.77 (m, 2H), 2.09-1.99 (m, 1H), 1.88-1.83 (m, 1H), 1.47 (s, 9H)

### Preparation B1-8: tert-butyl ((3R,4R)-1-(2-chloro-5-(1-(difluoromethyl)-1H-pyrazol-4-yl)pyridin-4-yl)-4-hydroxypiperidin-3-yl)carbamate

The titled compound was prepared in accordance with the same procedures as in Preparation B1-1, changing the amine reagent to tert-butyl N-[(3R,4R)-4-hydroxy-3-piperidyl]carbamate. 1H NMR (CDCl₃, 400 MHz) δ 8.17 (s, 1H), 8.14 (s, 1H), 7.96 (s, 1H), 7.27 (t, 1H), 6.93 (s, 1H), 4.68-4.67 (m, 1H), 3.61-3.54 (m, 3H), 3.25 (d, 1H), 2.73-2.64 (m, 2H), 2.56 (t, 1H), 2.03-1.99 (m, 1H), 1.47 (s, 9H)

### Preparation B1-9: tert-butyl N-[(3R,4R)-1-[2-chloro-5-[1-(difluoromethyl)pyrazol-4-yl]-4-pyridyl]-3-hydroxy-4-piperidyl]carbamate

The titled compound was prepared in accordance with the same procedures as in Preparation B1-1, changing the amine reagent to tert-butyl N-[(3R,4R)-3-hydroxy-4-piperidyl]carbamate. 1H NMR (CDCl₃, 400 MHz) δ 8.17 (s, 1H), 8.11 (s, 1H), 7.96 (s, 1H), 7.45-7.10 (m, 1H), 6.95 (s, 1H), 4.75-4.60 (m, 1H), 4.05-3.95 (m, 1H), 3.60-3.40 (m, 3H), 3.30-3.20 (m, 1H), 2.67 (t, 1H), 2.57 (t, 1H), 2.00-1.90 (m, 1H), 1.49 (s, 9H)

### Preparation B1-10: tert-butyl N-[(3R,4S)-1-[2-chloro-5-[1-(difluoromethyl)pyrazol-4-yl]-4-pyridyl]-3-methyl-4-piperidyl]carbamate

The titled compound was prepared in accordance with the same procedures as in Preparation B1-1, changing the amine reagent to tert-butyl N-[(3R,4S)-3-methyl-4-piperidyl]carbamate. 1H NMR (CDCl₃, 400 MHz) δ 8.14 (s, 1H), 8.12 (s, 1H), 7.95 (s, 1H), 7.45-7.10 (m, 1H), 6.92 (s, 1H), 4.65-4.55 (m, 1H), 3.90-3.80 (m, 1H), 3.05-2.90 (m, 2H), 2.90-2.75 (m, 2H), 2.25-2.15 (m, 1H), 1.75-1.65 (m, 1H), 1.46 (s, 9H), 0.94 (d, 3H)

### Preparation B2-1: 1-(2-chloro-5-iodopyridin-4-yl)-N,N-dimethylpyrrolidin-3-amine

2-chloro-4-fluoro-5-iodopyridine (200 mg, 0.78 mmol) was dissolved in anhydrous dimethylformamide (5.0 mL) and then 3-(dimethylamino)pyrrolidine (88.7 mg, 0.77 mmol) and diisopropylethylamine (0.32 mL, 2.33 mmol) were added dropwise thereto. The reaction mixture was stirred at 50 °C for 4 hours. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (800 mg). (Yield: 60.8%) 1H NMR (CDCl₃, 400 MHz) δ 8.42 (s, 1H), 6.44 (s, 1H), 3.80-3.70 (m, 1H), 3.70-3.65 (m, 2H), 3.60 (t, 1H), 2.76-2.68 (m, 1H), 2.32 (s, 6H), 2.24-2.18 (m, 1H), 1.90-1.79 (m, 1H)

### Preparation B2-2: (1-(2-chloro-5-iodopyridin-4-yl)-3-methylpyrrolidin-3-yl)methanol

The titled compound was prepared in accordance with the same procedures as in Preparation B2-1, changing the amine reagent to (3-methylpyrrolidin-3-yl)methanol. 1H NMR (CDCl₃, 400 MHz) δ 8.42 (s, 1H), 6.45 (s, 1H), 3.79-3.69 (m, 2H), 3.63 (d, 1H), 3.57 (d, 2H), 3.30 (d, 1H), 2.02-1.95 (m, 1H), 1.75-1.68 (m, 1H), 1.18 (s, 3H)

### Preparation B2-3: 1-(1-(2-chloro-5-iodopyridin-4-yl)-4-fluoropiperidin-4-yl)-N,N-dimethylmethanamine

The titled compound was prepared in accordance with the same procedures as in Preparation B2-1, changing the amine reagent to 1-(4-fluoro-4-piperidyl)-N,N-dimethyl-methanamine. 1H NMR (CDCl₃, 400 MHz) δ 8.56 (s, 1H), 6.88 (s, 1H), 3.37 (d, 2H), 3.07 (t, 2H), 2.55 (s, 1H), 2.49 (s, 1H), 2.35 (s, 6H), 2.10 (t, 2H), 1.95-1.75 (m, 2H)

### Preparation B2-4: (1-(2-chloro-5-iodopyridin-4-yl)-4-(2,2-difluoroethyl)piperidin-4-yl)methanol

The titled compound was prepared in accordance with the same procedures as in Preparation B2-1, changing the amine reagent to [4-(2,2-difluoroethyl)-4-piperidyl]methanol. 1H NMR (CDCl₃, 400 MHz) δ 8.55 (s, 1H), 6.86 (s, 1H), 6.29-5.98 (m, 1H), 3.69 (d, 2H), 3.21-3.09 (m, 4H), 2.12-2.01 (m, 2H), 1.83-1.74 (m, 4H), 1.60 (t, 1H)

### Preparation B2-5: (S)-1-(2-chloro-5-iodopyridin-4-yl)piperidin-3-carboxamide

The titled compound was prepared in accordance with the same procedures as in Preparation B2-1, changing the amine reagent to (3S)-piperidin-3-carboxamide. 1H NMR (CDCl₃, 400 MHz) δ 8.57 (s, 1H), 6.90 (s, 1H), 5.99 (s, 1H), 5.49 (s, 1H), 3.60-3.50 (m, 1H), 3.30-3.20 (m, 1H), 3.05-2.85 (m, 2H), 2.80-2.65 (m, 1H), 2.10-1.90 (m, 2H), 1.90-1.75 (m, 2H)

### Preparation B2-6: (R)-1-(2-chloro-5-iodopyridin-4-yl)piperidin-3-carboxamide

The titled compound was prepared in accordance with the same procedures as in Preparation B2-1, changing the amine reagent to (3R)-piperidin-3-carboxamide. 1H NMR (CDCl₃, 400 MHz) δ 8.58 (s, 1H), 6.90 (s, 1H), 5.98 (s, 1H), 5.43 (s, 1H), 3.60-3.50 (m, 1H), 3.30-3.20 (m, 1H), 3.05-2.85 (m, 2H), 2.75-2.65 (m, 1H), 2.10-1.90 (m, 2H), 1.90-1.75 (m, 2H)

### Preparation B2-7: tert-butyl (R)-(1-(2-chloro-5-iodopyridin-4-yl)piperidin-3-yl) carbamate

The titled compound was prepared in accordance with the same procedures as in Preparation B2-1, changing the amine reagent to (R)-3-(tert-butoxycarbonylamino)piperidine. 1H NMR (CDCl₃, 400 MHz) δ 8.56 (s, 1H), 6.84 (s, 1H), 4.92 (s, 1H), 3.93 (s, 1H), 3.40-3.25 (m, 1H), 3.15-2.95 (m, 2H), 2.95-2.80 (m, 1H), 2.00-1.70 (m, 3H), 1.65-1.50 (m, 1H), 1.46 (s, 9H)

### Preparation B2-8: 2-(1-(2-chloro-5-iodopyridin-4-yl)piperidin-4-yl)-N,N-dimethylethan-1-amine

The titled compound was prepared in accordance with the same procedures as in Preparation B2-1, changing the amine reagent to dimethyl[2-(piperidin-4-yl)ethyl]amine. 1H NMR (CDCl₃, 400 MHz) δ 8.53 (s, 1H), 6.81 (s, 1H), 3.53 (d, 2H), 2.71 (t, 2H), 2.33 (t, 2H), 2.24 (s, 6H), 1.84 (d, 2H), 1.51-1.43 (m, 5H)

### Preparation B2-9: 1-(1-(2-chloro-5-iodopyridin-4-yl)piperidin-4-yl)cyclopropan-1-ol

The titled compound was prepared in accordance with the same procedures as in Preparation B2-1, changing the amine reagent to 1-(piperidin-4-yl)cyclopropan-1-ol. 1H NMR (CDCl₃, 400 MHz) δ 8.55 (s, 1H), 6.83 (s, 1H), 3.64 (d, 2H), 2.69 (t, 2H), 1.90-1.70 (m, 5H), 1.25-1.15 (m, 1H), 0.80 (t, 2H), 0.55 (t, 2H)

### Preparation B2-10: 1-(1-(2-chloro-5-iodopyridin-4-yl)piperidin-4-yl)ethan-1-ol

The titled compound was prepared in accordance with the same procedures as in Preparation B2-1, changing the amine reagent to 1-(piperidin-4-yl)ethan-1-ol. 1H NMR (CDCl₃, 400 MHz) δ 8.54 (s, 1H), 6.83 (s, 1H), 3.71 (s, 1H), 3.70-3.64 (m, 1H), 3.61 (d, 2H), 2.69 (t, 2H), 2.02 (d, 1H), 1.78 (d, 1H), 1.65-1.40 (m, 2H), 1.39 (d, 1H), 1.26 (d, 3H)

### Preparation B2-11: 2-(1-(2-chloro-5-iodopyridin-4-yl)piperidin-4-yl)-2-methylpropan-1-ol

The titled compound was prepared in accordance with the same procedures as in Preparation B2-1, changing the amine reagent to 2-methyl-2-(piperidin-4-yl)propan-1-ol. 1H NMR (CDCl₃, 400 MHz) δ 8.53 (s, 1H), 6.81 (s, 1H), 3.63 (d, 2H), 3.46 (d, 2H), 2.67 (t, 2H), 1.80 (d, 2H), 1.65-1.45 (m, 2H), 1.33 (t, 1H), 0.92 (s, 6H)

### Preparation B2-12: 1-(2-chloro-5-iodopyridin-4-yl)-4-methylpiperazine

The titled compound was prepared in accordance with the same procedures as in Preparation B2-1, changing the amine reagent to 1-methylpiperazine. 1H NMR (CDCl₃, 400 MHz) δ 8.55 (s, 1H), 6.84 (s, 1H), 3.21 (s, 4H), 2.64 (s, 4H), 2.38 (s, 3H)

### Preparation B2-13: 2-chloro-5-iodo-4-(3-(2,2,2-trifluoroethyl)piperidin-1-yl)pyridine

The titled compound was prepared in accordance with the same procedures as in Preparation B2-1, changing the amine reagent to 3-(2,2,2-trifluoroethyl)piperidine. 1H NMR (CDCl₃, 400 MHz) δ 8.56 (s, 1H), 6.83 (s, 1H), 3.56 (d, 1H), 3.34 (d, 1H), 2.80 (t, 1H), 2.50 (t, 1H), 2.30-2.20 (m, 1H), 2.20-2.05 (m, 2H), 1.99 (d, 1H), 1.90-1.70 (m, 2H), 1.40-1.20 (m, 1H)

### Preparation B2-14: 2-chloro-4-(3-(fluoromethyl)piperidin-1-yl)-5-iodopyridine

The titled compound was prepared in accordance with the same procedures as in Preparation B2-1, changing the amine reagent to 3-(fluoromethyl)piperidine. 1H NMR (CDCl₃, 400 MHz) δ 8.56 (s, 1H), 6.85 (s, 1H), 4.55-4.25 (m, 2H), 3.56 (d, 1H), 3.42 (d, 1H), 2.77 (t, 1H), 2.64 (t, 1H), 2.35-2.15 (m, 1H), 1.95-1.75 (m, 3H), 1.40-1.20 (m, 1H)

### Preparation B2-15: N-(1-(2-chloro-5-iodopyridin-4-yl)piperidin-3-yl)acetamide

The titled compound was prepared in accordance with the same procedures as in Preparation B2-1, changing the amine reagent to 3-acetamidopiperidine. 1H NMR (CDCl₃, 400 MHz) δ 8.59 (s, 1H), 6.87 (s, 1H), 6.04 (s, 1H), 4.35-4.25 (m, 1H), 3.25 (d, 1H), 3.20-3.10 (m, 1H), 3.05-2.95 (m, 2H), 2.03 (s, 3H), 2.00-1.85 (m, 1H), 1.85-1.65 (m, 3H)

### Preparation B2-16: 2-chloro-5-iodo-4-(4-(trifluoromethyl)piperidin-1-yl)pyridine

The titled compound was prepared in accordance with the same procedures as in Preparation B2-1, changing the amine reagent to 4-(trifluoromethyl)piperidine. 1H NMR (CDCl₃, 400 MHz) δ 8.58 (s, 1H), 6.85 (s, 1H), 3.63 (d, 2H), 2.73 (t, 2H), 2.35-2.20 (m, 1H), 2.10-2.00 (m, 2H), 1.95-1.80 (m, 2H)

### Preparation B2-17: tert-butyl 8-(2-chloro-5-iodopyridin-4-yl)-2,8-diazaspiro[4.5]decan-2-carboxylate

The titled compound was prepared in accordance with the same procedures as in Preparation B2-1, changing the amine reagent to tert-butyl 2,8-diazaspiro[4.5]decan-2-carboxylate. 1H NMR (CDCl₃, 400 MHz) δ 8.55 (s, 1H), 6.85 (d, 1H), 3.50-3.40 (m, 2H), 3.40-3.10 (m, 4H), 3.10-2.95 (m, 2H), 1.90-1.70 (m, 6H), 1.48 (s, 9H)

### Preparation B2-18: tert-butyl 2-(2-chloro-5-iodopyridin-4-yl)-2,8-diazaspiro[4.5]decan-8-carboxylate

The titled compound was prepared in accordance with the same procedures as in Preparation B2-1, changing the amine reagent to tert-butyl 2,8-diazaspiro[4.5]decan-8-carboxylate. 1H NMR (CDCl₃, 400 MHz) δ 8.42 (s, 1H), 6.44 (s, 1H), 3.72 (t, 2H), 3.55-3.45 (m, 4H), 3.45-3.35 (m, 2H), 1.87 (t, 2H), 1.65-1.50 (m, 4H), 1.48 (s, 9H)

### Preparation B2-19: (3R,4R)-1-(2-chloro-5-iodopyridin-4-yl)-4-fluoropiperidin-3-ol

The titled compound was prepared in accordance with the same procedures as in Preparation B2-1, changing the amine reagent to tert-butyl (3R,4R)-4-fluoropiperidin-3-ol. 1H NMR (CDCl₃, 400 MHz) δ 8.60 (s, 1H), 6.92 (s, 1H), 4.70-4.50 (m, 1H), 4.10-4.00 (m, 1H), 3.51 (d, 1H), 3.35-3.20 (m, 1H), 3.10-3.00 (m, 2H), 2.60 (d, 1H), 2.40-2.25 (m, 1H), 2.10-2.00 (m, 1H)

### Preparation B2-20: (3R,4R)-1-(2-chloro-5-iodopyridin-4-yl)-4-(hydroxymethyl)piperidin-3-ol

The titled compound was prepared in accordance with the same procedures as in Preparation B2-1, changing the amine reagent to trans-4-(hydroxymethyl)-3-piperidinol hydrochloride. 1H NMR (CDCl₃, 400 MHz) δ 8.56 (s, 1H), 6.86 (s, 1H), 3.95-3.85 (m, 2H), 3.85-3.75 (m, 1H), 3.68 (d, 1H), 3.51 (d, 1H), 3.32 (s, 1H), 2.74 (t, 1H), 2.59 (t, 1H), 2.24 (t, 1H), 1.85-1.70 (m, 2H), 1.55-1.40 (m, 1H)

### Preparation B2-21: tert-butyl (R)-(1-(2-chloro-5-iodopyridin-4-yl)pyrrolidin-3-yl) carbamate

The titled compound was prepared in accordance with the same procedures as in Preparation B2-1, changing the amine reagent to (R)-3-(tert-butoxycarbonylamino)pyrrolidine. 1H NMR (CDCl₃, 400 MHz) δ 8.43 (s, 1H), 6.46 (s, 1H), 4.70 (s, 1H), 4.32 (s, 1H), 3.90-3.80 (m, 1H), 3.80 (q, 1H), 3.62 (q, 1H), 3.55-3.50 (m, 1H), 2.28-2.19 (m, 1H), 2.00-1.90 (m, 1H), 1.47 (s, 9H)

### Preparation B2-22: tert-butyl (S)-(1-(2-chloro-5-iodopyridin-4-yl)pyrrolidin-3-yl) carbamate

The titled compound was prepared in accordance with the same procedures as in Preparation B2-1, changing the amine reagent to (S)-3-(tert-butoxycarbonylamino)pyrrolidine. 1H NMR (CDCl₃, 400 MHz) δ 8.43 (s, 1H), 6.46 (s, 1H), 4.71 (s, 1H), 4.32 (s, 1H), 3.90-3.85 (m, 1H), 3.78 (q, 1H), 3.62 (q, 1H), 3.55-3.50 (m, 1H), 2.28-2.19 (m, 1H), 2.00-1.90 (m, 1H), 1.47 (s, 9H)

### Preparation B2-23: 8-(2-chloro-5-iodopyridin-4-yl)-2-methyl-2,8-diazaspiro[4.5]decane

The titled compound was prepared in accordance with the same procedures as in Preparation B2-1, changing the amine reagent to 2-methyl-2,8-diazaspiro[4.5]decane. 1H NMR (CD₃OD, 400 MHz) δ 8.50 (s, 1H), 7.02 (d, 1H), 3.20-3.10 (m, 4H), 2.68 (t, 2H), 2.55 (s, 2H), 2.39 (s, 3H), 1.90-1.75 (m, 6H)

### Preparation B2-24: (3S)-1-(2-chloro-5-iodo-4-pyridyl)-N-methyl-pyrrolidin-3-amine

The titled compound was prepared in accordance with the same procedures as in Preparation B2-1, changing the amine reagent to (S)-3-(methylamino)pyrrolidine. 1H NMR (CDCl₃, 400 MHz) δ 8.42 (s, 1H), 6.45 (s, 1H), 3.86-3.81 (m, 1H), 3.73 (q, 1H), 3.62 (q, 1H), 3.51-3.47 (m, 1H), 3.40-3.30 (m, 1H), 2.48 (s, 3H), 2.19-2.11 (m, 1H), 1.90-1.82 (m, 1H)

### Preparation B2-25: (3S)-1-(2-chloro-5-iodo-4-pyridyl)-N-ethyl-pyrrolidin-3-amine

The titled compound was prepared in accordance with the same procedures as in Preparation B2-1, changing the amine reagent to (S)-3-(ethylamino)pyrrolidine. 1H NMR (CDCl₃, 400 MHz) δ 8.41 (s, 1H), 6.44 (s, 1H), 3.86-3.81 (m, 1H), 3.73-3.50 (m, 2H), 3.50-3.40 (m, 2H), 2.80-2.65 (m, 2H), 2.22-2.14 (m, 1H), 1.87-1.79 (m, 1H), 1.27 (t, 3H)

### Preparation B2-26: (R)-N-(1-(2-chloro-5-iodopyridin-4-yl)pyrrolidin-3-yl)acetamide

The titled compound was prepared in accordance with the same procedures as in Preparation B2-1, changing the amine reagent to (R)-3-acetamidopyrrolidine. 1H NMR (CDCl₃, 400 MHz) δ 8.41 (s, 1H), 6.45 (s, 1H), 6.05-5.95 (m, 1H), 4.65-4.55 (m, 1H), 3.96-3.91 (m, 1H), 3.78 (q, 1H), 3.59-3.53 (m, 2H), 2.30-2.20 (m, 1H), 2.10-2.00 (m, 1H), 2.03 (s, 3H)

### Preparation B2-27: (S)-N-(1-(2-chloro-5-iodopyridin-4-yl)pyrrolidin-3-yl)acetamide

The titled compound was prepared in accordance with the same procedures as in Preparation B2-1, changing the amine reagent to (S)-3-acetamidopyrrolidine. 1H NMR (CDCl₃, 400 MHz) δ 8.41 (s, 1H), 6.45 (s, 1H), 6.00-5.90 (m, 1H), 4.65-4.55 (m, 1H), 4.14-3.91 (m, 1H), 3.78 (q, 1H), 3.58-3.53 (m, 2H), 2.30-2.20 (m, 1H), 2.10-1.98 (m, 4H)

### Preparation B2-28: 1-(2-chloro-5-iodo-4-pyridyl)piperidin-3-carboxamide

The titled compound was prepared in accordance with the same procedures as in Preparation B2-1, changing the amine reagent to piperidin-3-carboxamide. 1H NMR (CDCl₃, 400 MHz) δ 8.58 (s, 1H), 6.90 (s, 1H), 5.98 (s, 1H), 5.41 (s, 1H), 3.56 (d, 1H), 3.30-3.20 (m, 1H), 3.10-2.90 (m, 2H), 2.75-2.65 (m, 1H), 2.10-1.90 (m, 2H), 1.90-1.75 (m, 2H)

### Preparation B2-29: (1S,4S)-4-((2-chloro-5-iodopyridin-4-yl)(methyl)amino)cyclohexan-1-ol

The titled compound was prepared in accordance with the same procedures as in Preparation B2-1, changing the amine reagent to cis-4-(methylamino)cyclohexanol. 1H NMR (CDCl₃, 400 MHz) δ 8.55 (s, 1H), 6.82 (s, 1H), 4.08 (brs, 1H), 3.59-3.53 (m, 1H), 2.80 (s, 3H), 2.05-2.00 (m, 2H), 1.92-1.88 (m, 2H), 1.65-1.57 (m, 4H), 1.27-1.27 (m, 1H)

### Preparation B2-30: tert-butyl N-[(3S,6R)-1-(2-chloro-5-iodo-4-pyridyl)-6-methyl-3-piperidyl]carbamate

The titled compound was prepared in accordance with the same procedures as in Preparation B2-1, changing the amine reagent to tert-butyl N-[(3S,6R)-6-methyl-3-piperidyl]carbamate. 1H NMR (CDCl₃, 400 MHz) δ 8.71 (s, 1H), 7.00 (s, 1H), 5.20 (s, 1H), 3.94 (s, 1H), 3.73 (s, 1H), 3.11-2.89 (m, 2H), 2.04-1.87 (m, 1H), 1.87-1.63 (m, 3H), 1.54 (s, 9H), 1.08 (d, 3H)

### Preparation B2-31: tert-butyl N-[(3R,4S)-1-(2-chloro-5-iodo-4-pyridyl)-4-methoxy-3-piperidyl]carbamate

The titled compound was prepared in accordance with the same procedures as in Preparation B2-1, changing the amine reagent to tert-butyl N-[(3R,4S)-4-methoxy-3-piperidyl]carbamate. 1H NMR (CDCl₃, 400 MHz) δ 8.62 (s, 1H), 6.92 (s, 1H), 5.27 (d, 1H), 4.11-3.98 (m, 1H), 3.64-3.52 (m, 1H), 3.48 (s, 3H), 3.34-3.25 (m, 1H), 3.23-3.12 (m, 1H), 3.07-2.93 (m, 2H), 2.19-2.08 (m, 1H), 1.97-1.81 (m, 1H), 1.56 (s, 9H)

### Preparation B2-32: tert-butyl N-[(3R,4R)-1-(2-chloro-5-iodo-4-pyridyl)-4-methoxy-3-piperidyl]carbamate

The titled compound was prepared in accordance with the same procedures as in Preparation B2-1, changing the amine reagent to tert-butyl N-[(3R,4R)-4-methoxypiperidin-3-yl]carbamate. 1H NMR (CDCl₃, 400 MHz) δ 8.66 (s, 1H), 6.96 (s, 1H), 5.38 (s, 1H), 4.05 (s, 1H), 3.50 (s, 3H), 3.45-3.38 (m, 2H), 3.16-3.02 (m, 3H), 2.25-2.08 (m, 1H), 1.95-1.83 (m, 1H), 1.55 (s, 9H)

### Preparation B2-33: tert-butyl N-methyl-N-[[(3R)-1-(2-chloro-5-iodo-4-pyridyl)-3-piperidyl]methyl]carbamate

The titled compound was prepared in accordance with the same procedures as in Preparation B2-1, changing the amine reagent to tert-butyl N-methyl-N-([(3R)-piperidin-3-yl]methyl)carbamate. 1H NMR (CDCl₃, 400 MHz) δ 8.55 (s, 1H), 6.82 (s, 1H), 3.55-3.45 (m, 1H), 3.45-3.35 (m, 1H), 3.35-3.15 (m, 1H), 3.15-3.05 (m, 1H), 2.91 (s, 3H), 2.80-2.65 (m, 1H), 2.55-2.40 (m, 1H), 2.25-2.10 (m, 1H), 1.95-1.70 (m, 3H), 1.48 (s, 9H), 1.25-1.10 (m, 1H)

### Preparation B2-34: tert-butyl N-methyl-N-[[(3S)-1-(2-chloro-5-iodo-4-pyridyl)-3-piperidyl]methyl]carbamate

The titled compound was prepared in accordance with the same procedures as in Preparation B2-1, changing the amine reagent to tert-butyl N-methyl-N-([(3S)-piperidin-3-yl]methyl)carbamate. 1H NMR (CDCl₃, 400 MHz) δ 8.54 (s, 1H), 6.82 (s, 1H), 3.55-3.45 (m, 1H), 3.45-3.35 (m, 1H), 3.35-3.20 (m, 1H), 3.20-3.05 (m, 1H), 2.90 (s, 3H), 2.85-2.65 (m, 1H), 2.50-2.40 (m, 1H), 2.25-2.10 (m, 1H), 1.95-1.70 (m, 3H), 1.48 (s, 9H), 1.25-1.10 (m, 1H)

### Preparation B2-35: tert-butyl N-[(3R,4R)-1-(2-chloro-5-iodo-4-pyridyl)-3-methyl-4-piperidyl]carbamate

The titled compound was prepared in accordance with the same procedures as in Preparation B2-1, changing the amine reagent to tert-butyl N-[(3R,4R)-3-methyl-4-piperidyl]carbamate. 1H NMR (CDCl₃, 400 MHz) δ 8.55 (s, 1H), 6.84 (s, 1H), 4.50-4.40 (m, 1H), 3.60-3.50 (m, 2H), 2.85-2.75 (m, 1H), 2.55-2.45 (m, 1H), 2.20-2.10 (m, 1H), 1.85-1.70 (m, 1H), 1.49 (s, 9H), 1.04 (d, 3H)

### Preparation B2-36: 2-chloro-5-iodo-4-[(3S)-3-methyl-1-piperidyl]pyridine

The titled compound was prepared in accordance with the same procedures as in Preparation B2-1, changing the amine reagent to (3S)-3-methylpiperidine. 1H NMR (CDCl₃, 400 MHz) δ 12.40 (s, 1H), 10.90 (s, 1H), 7.50-7.40 (m, 2H), 6.69 (t, 1H), 6.33 (t, 1H), 5.90-5.70 (m, 4H), 5.20-5.10 (m, 1H), 4.92 (d, 3H)

### Preparation B2-37: 2-chloro-5-iodo-4-[(3S)-3-(fluoromethyl)-1-piperidyl]pyridine

The titled compound was prepared in accordance with the same procedures as in Preparation B2-1, changing the amine reagent to (3S)-3-(fluoromethyl)piperidine hydrochloride. 1H NMR (CDCl₃, 400 MHz) δ 12.50 (s, 1H), 11.00 (s, 1H), 8.45-8.35 (m, 1H), 8.35-8.20 (m, 1H), 7.60-7.50 (m, 1H), 7.45-7.35 (m, 1H), 6.78 (t, 1H), 6.60 (t, 1H), 6.25-6.10 (m, 1H), 5.90-5.70 (m, 3H), 5.35-5.20 (m, 1H)

### Preparation B2-38: 2-chloro-5-iodo-4-[(3R)-3-(difluoromethyl)-1-piperidyl]pyridine

The titled compound was prepared in accordance with the same procedures as in Preparation B2-1, changing the amine reagent to (3R)-3-(difluoromethyl)piperidine hydrochloride. 1H NMR (CDCl₃, 400 MHz) δ 12.50 (s, 1H), 11.00 (s, 1H), 10.00-9.60 (m, 1H), 7.65-7.50 (m, 1H), 7.40-7.30 (m, 1H), 6.78 (t, 1H), 6.68 (t, 1H), 6.30-6.15 (m, 1H), 6.00-5.80 (m, 2H), 5.80-5.65 (m, 1H), 5.50-5.35 (m, 1H)

### Preparation B2-39: tert-butyl N-[(3S,4S)-1-(2-chloro-5-iodo-4-pyridyl)-3-fluoro-4-piperidyl]carbamate

The titled compound was prepared in accordance with the same procedures as in Preparation B2-1, changing the amine reagent to tert-butyl N-[(3S,4S)-3-fluoro-4-piperidyl]carbamate. 1H NMR (CDCl₃, 400 MHz) δ 8.34 (s, 1H), 6.60 (s, 1H), 4.85-4.70 (m, 1H), 4.50-4.30 (m, 1H), 4.05-3.90 (m, 1H), 3.70-3.60 (m, 1H), 3.20-3.05 (m, 1H), 2.20-2.10 (m, 1H), 1.65-1.55 (m, 1H), 1.51 (s, 9H), 1.30-1.20 (m, 1H)

### Preparation B2-40: tert-butyl N-[(3R,4S)-1-(2-chloro-5-iodo-4-pyridyl)-3-fluoro-4-piperidyl]carbamate

The titled compound was prepared in accordance with the same procedures as in Preparation B2-1, changing the amine reagent to tert-butyl N-[(3R,4S)-3-fluoro-4-piperidyl]carbamate. 1H NMR (CDCl₃, 400 MHz) δ 8.58 (s, 1H), 6.88 (s, 1H), 5.00-4.90 (m, 1H), 4.90-4.70 (m, 1H), 3.95-3.75 (m, 1H), 3.75-3.65 (m, 1H), 3.20-3.00 (m, 1H), 2.87 (t, 1H), 2.20-2.00 (m, 1H), 1.95-1.85 (m, 1H), 1.49 (s, 9H)

### Preparation B2-41: tert-butyl N-[(3S,4R)-1-(2-chloro-5-iodo-4-pyridyl)-3-fluoro-4-piperidyl]carbamate

The titled compound was prepared in accordance with the same procedures as in Preparation B2-1, changing the amine reagent to tert-butyl N-[(3S,4R)-3-fluoro-4-piperidyl]carbamate. 1H NMR (CDCl₃, 400 MHz) δ 8.58 (s, 1H), 6.88 (s, 1H), 5.00-4.90 (m, 1H), 4.90-4.75 (m, 1H), 3.95-3.75 (m, 1H), 3.75-3.60 (m, 1H), 3.20-3.00 (m, 1H), 2.87 (t, 1H), 2.20-2.00 (m, 1H), 1.95-1.85 (m, 1H), 1.49 (s, 9H)

### Preparation B2-42: tert-butyl N-[(3S,5R)-1-(2-chloro-5-iodo-4-pyridyl)-5-fluoro-3-piperidyl]carbamate

The titled compound was prepared in accordance with the same procedures as in Preparation B2-1, changing the amine reagent to tert-butyl N-[(3S,5R)-5-fluoro-3-piperidyl]carbamate. 1H NMR (CDCl₃, 400 MHz) δ 8.68 (s, 1H), 6.98 (s, 1H), 5.28-5.08 (m, 1H), 5.07-4.84 (m, 1H), 4.16-3.98 (m, 1H), 3.45-3.28 (m, 2H), 3.28-3.11 (m, 2H), 2.32-2.13 (m, 1H), 2.07-1.87 (m, 1H), 1.64-1.54 (m, 1H), 1.54 (s, 9H)

### Preparation B2-43: tert-butyl N-[(3S,5S)-1-(2-chloro-5-iodo-4-pyridyl)-5-fluoro-3-piperidyl]carbamate

The titled compound was prepared in accordance with the same procedures as in Preparation B2-1, changing the amine reagent to tert-butyl N-[(3S,5S)-5-fluoro-3-piperidyl]carbamate. 1H NMR (CDCl₃, 400 MHz) δ 8.65 (s, 1H), 6.98 (s, 1H), 5.10-5.00 (m, 1H), 4.98-4.90 (m, 1H), 4.90-4.80 (m, 1H), 4.31-4.18 (m, 1H), 3.49-3.35 (m, 2H), 3.30-3.15 (m, 1H), 3.05-2.92 (m, 1H), 2.15-2.01 (m, 2H), 1.54 (s, 9H)

### Preparation B2-44: tert-butyl N-[(3S,5R)-1-(2-chloro-5-iodo-4-pyridyl)-5-(trifluoromethyl)-3-piperidyl]carbamate

The titled compound was prepared in accordance with the same procedures as in Preparation B2-1, changing the amine reagent to tert-butyl N-[(3S,5R)-5-(trifluoromethyl)-3-piperidyl]carbamate. 1H NMR (CDCl₃, 400 MHz) δ 8.66 (s, 1H), 6.93 (s, 1H), 4.59-4.39 (m, 1H), 4.04-3.85 (m, 1H), 3.81-3.65 (m, 2H), 2.86-2.69 (m, 1H), 2.69-2.56 (m, 1H), 2.52-2.33 (m, 2H), 1.54 (s, 9H)

### Preparation B2-45: tert-butyl N-[(3S,5S)-1-(2-chloro-5-iodo-4-pyridyl)-5-hydroxy-3-piperidyl]carbamate

The titled compound was prepared in accordance with the same procedures as in Preparation B2-1, changing the amine reagent to tert-butyl N-[(3S,5S)-5-hydroxy-3-piperidyl]carbamate. 1H NMR (CDCl₃, 400 MHz) δ 8.65 (s, 1H), 6.96 (s, 1H), 5.33-5.13 (m, 1H), 4.22-4.07 (m, 1H), 4.05-3.89 (m, 1H), 3.43-3.27 (m, 2H), 3.07-2.95 (m, 2H), 2.24-2.14 (m, 1H), 2.14-2.06 (m, 1H), 1.70-1.61 (m, 1H), 1.53 (s, 9H)

### Preparation B2-46: tert-butyl N-[(3R,4R)-1-(2-chloro-5-iodo-4-pyridyl)-3-hydroxy-4-piperidyl]carbamate

The titled compound was prepared in accordance with the same procedures as in Preparation B2-1, changing the amine reagent to tert-butyl ((3R,4R)-3-hydroxypiperidin-4-yl)carbamate. 1H NMR (CDCl₃, 400 MHz) δ 8.58 (s, 1H), 6.89 (s, 1H), 4.80-4.65 (m, 1H), 4.10-3.95 (m, 1H), 3.80-3.65 (m, 2H), 3.60-3.45 (m, 2H), 2.82 (t, 1H), 2.66 (t, 1H), 2.20-2.05 (m, 1H), 1.80-1.65 (m, 1H), 1.50 (s, 9H)

### Preparation B2-47: tert-butyl ((3R,4R)-1-(2-chloro-5-iodopyridin-4-yl)-4-fluoropiperidin-3-yl)carbamate

The titled compound was prepared in accordance with the same procedures as in Preparation B2-1, changing the amine reagent to tert-butyl N-[(3R,4R)-4-fluoropiperidin-3-yl]carbamate. 1H NMR (CDCl₃, 400 MHz) 8.68 (s, 1H), 7.00 (s, 1H), 5.31-5.03 (m, 1H), 4.86-4.72 (m, 0.5H), 4.74-4.59 (m, 0.5H), 4.20-3.87 (m, 1H), 3.52-3.32 (m, 1H), 3.25-3.02 (m, 3H), 2.41-2.16 (m, 1H), 2.16-1.96 (m, 1H), 1.48 (s, 9H)

### Preparation B3-1: 1-(2-chloro-5-(1-(tetrahydro-2H-pyran-4-yl)-1H-pyrazol-4-yl)pyridin-4-yl)-N,N-dimethylpyrrolidin-3-amine

1-(2-chloro-5-iodopyridin-4-yl)-N,N-dimethylpyrrolidin-3-amine (the B2-1 intermediate, 44.2 mg, 0.13 mmol) was dissolved in anhydrous 1,4-dioxane (1.5 mL) and then 1-(tetrahydro-2H-pyran-4-yl)-1H-pyrazol-4-boronic acid pinacol ester (35.0 mg, 0.13 mmol), [1,1′-bis(diphenylphosphino)ferrocene]dichloropalladium(II)-dichloromethane complex (5.1 mg, 0.006 mmol), and 2.0M sodium carbonate solution (0.3 mL, 0.6 mmol) were added dropwise thereto. Then, the mixed solution was stirred at 80 °C overnight. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (24.4 mg). (Yield: 51.6%) 1H NMR (CDCl₃, 400 MHz) δ 7.87 (s, 1H), 7.52 (s, 1H), 7.43 (s, 1H), 6.53 (s, 1H), 4.42-4.33 (m, 1H), 4.13 (d, 2H), 3.57 (t, 2H), 3.26-3.14 (m, 3H), 2.95 (t, 1H), 2.64-3.32 (m, 1H), 2.19 (s, 6H), 2.15-2.00 (m, 5H), 1.80-1.65 (m, 1H)

### Preparation B3-2: (1-(2-chloro-5-(1-(tetrahydro-2H-pyran-4-yl)-1H-pyrazol-4-yl)pyridin-4-yl)-3-methylpyrrolidin-3-yl)methanol

The titled compound was prepared in accordance with the same procedures as in Preparation B3-1, using the B2-2 intermediate. 1H NMR (CDCl₃, 400 MHz) δ 7.87 (s, 1H), 7.51 (s, 1H), 7.44 (s, 1H), 6.55 (s, 1H), 4.43-4.35 (m, 1H), 4.14 (d, 2H), 3.58 (t, 2H), 3.46 (d, 2H), 3.15 (t, 2H), 3.11 (d, 1H), 2.79 (d, 1H), 2.20-2.00 (m, 4H), 1.88-1.80 (m, 1H), 1.62-1.55 (m, 2H), 1.55-1.50 (m, 1H), 1.08 (s, 3H)

### Preparation B3-3: 1-(1-(2-chloro-5-(1-(tetrahydro-2H-pyran-4-yl)-1H-pyrazol-4-yl)pyridin-4-yl)-4-fluoropiperidin-4-yl)-N,N-dimethylmethanamine

The titled compound was prepared in accordance with the same procedures as in Preparation B3-1, using the B2-3 intermediate. 1H NMR (CDCl₃, 400 MHz) δ 8.12 (s, 1H), 7.85 (s, 1H), 7.74 (s, 1H), 6.90 (s, 1H), 4.44-4.38 (m, 1H), 4.15 (d, 2H), 3.56 (t, 2H), 3.15 (d, 2H), 2.95 (t, 2H), 2.50 (s, 1H), 2.44 (s, 1H), 2.32 (s, 6H), 2.30-2.24 (m, 1H), 2.20-2.10 (m, 4H), 2.00-1.90 (m, 2H), 1.70-1.60 (m, 1H)

### Preparation B3-4: (1-(2-chloro-5-(1-(tetrahydro-2H-pyran-4-yl)-1H-pyrazol-4-yl)pyridin-4-yl)-4-(2,2-difluoroethyl)piperidin-4-yl)methanol

The titled compound was prepared in accordance with the same procedures as in Preparation B3-1, using the B2-4 intermediate. 1H NMR (CDCl₃, 400 MHz) δ 8.12 (s, 1H), 7.84 (s, 1H), 7.76 (s, 1H), 6.88 (s, 1H), 6.24-5.93 (m, 1H), 4.44-4.37 (m, 1H), 4.15 (d, 2H), 3.64 (d, 2H), 3.58 (t, 2H), 3.05-2.90 (m, 4H), 2.20-1.95 (m, 7H), 1.65-1.55 (m, 4H), 1.27 (t, 1H)

### Preparation B3-5: (S)-1-(2-chloro-5-(1-(tetrahydro-2H-pyran-4-yl)-1H-pyrazol-4-yl)pyridin-4-yl)piperidin-3-carboxamide

The titled compound was prepared in accordance with the same procedures as in Preparation B3-1, using the B2-5 intermediate. 1H NMR (CDCl₃, 400 MHz) δ 8.12 (s, 1H), 7.83 (s, 1H), 7.75 (s, 1H), 6.88 (s, 1H), 5.45-5.40 (m, 1H), 5.25-5.15 (m, 1H), 4.45-4.40 (m, 1H), 4.15 (d, 2H), 3.58 (t, 2H), 3.43 (d, 1H), 3.21 (d, 1H), 2.79 (t, 1H), 2.65 (t, 1H), 2.55-2.40 (m, 1H), 2.37 (t, 1H), 2.20-2.05 (m, 4H), 2.05-1.90 (m, 3H)

### Preparation B3-6: (R)-1-(2-chloro-5-(1-(tetrahydro-2H-pyran-4-yl)-1H-pyrazol-4-yl)pyridin-4-yl)piperidin-3-carboxamide

The titled compound was prepared in accordance with the same procedures as in Preparation B3-1, using the B2-6 intermediate. 1H NMR (CDCl₃, 400 MHz) δ 8.12 (s, 1H), 7.83 (s, 1H), 7.75 (s, 1H), 6.89 (s, 1H), 5.45-5.35 (m, 1H), 5.25-5.15 (m, 1H), 4.50-4.30 (m, 1H), 4.15 (d, 2H), 3.58 (t, 2H), 3.43 (d, 1H), 3.22 (d, 1H), 2.79 (t, 1H), 2.66 (t, 1H), 2.50-2.40 (m, 1H), 2.20-2.05 (m, 4H), 2.05-1.90 (m, 1H), 1.85-1.75 (m, 1H), 1.75-1.60 (m, 1H)

### Preparation B3-7: tert-butyl(R)-(1-(2-chloro-5-(1-(tetrahydro-2H-pyran-4-yl)-1H-pyrazol-4-yl)pyridin-4-yl)piperidin-3-yl)carbamate

The titled compound was prepared in accordance with the same procedures as in Preparation B3-1, using the B2-7 intermediate. 1H NMR (CDCl₃, 400 MHz) δ 8.15 (s, 1H), 7.81 (s, 1H), 7.77 (s, 1H), 6.86 (s, 1H), 4.55-4.45 (m, 1H), 4.45-4.35 (m, 1H), 4.15 (d, 2H), 3.80-3.70 (m, 1H), 3.58 (t, 2H), 3.43 (d, 1H), 3.07 (d, 1H), 2.65-2.55 (m, 1H), 2.55-2.45 (m, 1H), 2.25-2.05 (m, 4H), 2.00-1.90 (m, 1H), 1.75-1.65 (m, 1H), 1.45 (s, 9H), 1.40-1.30 (m, 1H)

### Preparation B3-8: 2-(1-(2-chloro-5-(1-(tetrahydro-2H-pyran-4-yl)-1H-pyrazol-4-yl)pyridin-4-yl)piperidin-4-yl)-N,N-dimethylethan-1-amine

The titled compound was prepared in accordance with the same procedures as in Preparation B3-1, using the B2-8 intermediate. 1H NMR (CDCl₃, 400 MHz) δ 8.11 (s, 1H), 7.84 (s, 1H), 7.78 (s, 1H), 6.84 (s, 1H), 4.44-4.38 (m, 1H), 4.15 (d, 2H), 3.59 (t, 2H), 3.32 (d, 2H), 2.57 (t, 2H), 2.40-2.30 (m, 2H), 2.27 (s, 6H), 2.20-2.00 (m, 4H), 1.73 (d, 2H), 1.50-1.40 (m, 4H), 1.30-1.20 (m, 1H)

### Preparation B3-9: 1-(1-(2-chloro-5-(1-(tetrahydro-2H-pyran-4-yl)-1H-pyrazol-4-yl)pyridin-4-yl)piperidin-4-yl)cyclopropan-1-ol

The titled compound was prepared in accordance with the same procedures as in Preparation B3-1, using the B2-9 intermediate. 1H NMR (CDCl₃, 400 MHz) δ 8.13 (s, 1H), 7.85 (s, 1H), 7.82 (s, 1H), 6.89 (s, 1H), 4.50-4.35 (m, 1H), 4.13 (d, 2H), 3.58 (t, 2H), 3.42 (d, 2H), 2.55 (t, 2H), 2.20-2.05 (m, 4H), 1.90-1.70 (m, 5H), 1.20-1.05 (m, 1H), 0.79 (t, 2H), 0.50 (t, 2H)

### Preparation B3-10: 1-(1-(2-chloro-5-(1-(tetrahydro-2H-pyran-4-yl)-1H-pyrazol-4-yl)pyridin-4-yl)piperidin-4-yl)ethan-1-ol

The titled compound was prepared in accordance with the same procedures as in Preparation B3-1, using the B2-10 intermediate. 1H NMR (CDCl₃, 400 MHz) δ 8.12 (s, 1H), 7.84 (s, 1H), 7.79 (s, 1H), 6.86 (s, 1H), 4.44-4.35 (m, 1H), 4.14 (d, 2H), 3.65-3.50 (m, 4H), 3.45-3.35 (m, 2H), 2.56 (t, 2H), 2.20-2.05 (m, 4H), 1.90 (d, 1H), 1.65 (d, 1H), 1.45-1.35 (m, 2H), 1.35-1.25 (m, 1H), 1.21 (d, 3H)

### Preparation B3-11: 2-(1-(2-chloro-5-(1-(tetrahydro-2H-pyran-4-yl)-1H-pyrazol-4-yl)pyridin-4-yl)piperidin-4-yl)-2-methylpropan-1-ol

The titled compound was prepared in accordance with the same procedures as in Preparation B3-1, using the B2-11 intermediate. 1H NMR (CDCl₃, 400 MHz) δ 8.12 (s, 1H), 7.83 (s, 1H), 7.81 (s, 1H), 6.84 (s, 1H), 4.50-4.35 (m, 1H), 4.14 (d, 2H), 3.58 (t, 2H), 3.45-3.35 (m, 4H), 2.54 (t, 2H), 2.20-2.15 (m, 2H), 2.15-2.05 (m, 2H), 1.68 (d, 2H), 1.50-1.30 (m, 2H), 1.30-1.20 (m, 2H), 0.86 (s, 6H)

### Preparation B3-12: 1-(2-chloro-5-(1-(tetrahydro-2H-pyran-4-yl)-1H-pyrazol-4-yl)pyridin-4-yl)-4-methylpiperazine

The titled compound was prepared in accordance with the same procedures as in Preparation B3-1, using the B2-12 intermediate. 1H NMR (CDCl₃, 400 MHz) δ 8.13 (s, 1H), 7.84 (s, 1H), 7.78 (s, 1H), 6.86 (s, 1H), 4.45-4.35 (m, 1H), 4.16 (d, 2H), 3.59 (t, 2H), 3.01 (s, 4H), 2.47 (s, 4H), 2.33 (s, 3H), 2.20-2.05 (m, 4H)

### Preparation B3-13: 2-chloro-5-(1-(tetrahydro-2H-pyran-4-yl)-1H-pyrazol-4-yl)-4-(3-(2,2,2-trifluoroethyl)piperidin-1-yl)pyridine

The titled compound was prepared in accordance with the same procedures as in Preparation B3-1, using the B2-13 intermediate. 1H NMR (CDCl₃, 400 MHz) δ 8.12 (s, 1H), 7.80 (s, 1H), 7.75 (s, 1H), 6.85 (s, 1H), 4.44-4.35 (m, 1H), 4.15 (d, 2H), 3.58 (t, 2H), 3.41 (d, 1H), 3.26 (d, 1H), 2.60 (t, 1H), 2.25 (t, 1H), 2.20-2.05 (m, 4H), 2.05-1.80 (m, 4H), 1.80-1.70 (m, 1H), 1.70-1.65 (m, 1H), 1.20-1.05 (m, 1H)

### Preparation B3-14: 2-chloro-4-(3-(fluoromethyl)piperidin-1-yl)-5-(1-(tetrahydro-2H-pyran-4-yl)-1H-pyrazol-4-yl)pyridine

The titled compound was prepared in accordance with the same procedures as in Preparation B3-1, using the B2-14 intermediate. 1H NMR (CDCl₃, 400 MHz) δ 8.13 (s, 1H), 7.80 (s, 1H), 7.76 (s, 1H), 6.87 (s, 1H), 4.45-4.35 (m, 2H), 4.28-4.23 (m, 1H), 4.20-4.10 (m, 3H), 3.58 (t, 2H), 3.42 (d, 1H), 3.27 (d, 1H), 2.56 (t, 1H), 2.43 (t, 1H), 2.20-2.00 (m, 4H), 1.78 (t, 2H), 1.75-1.60 (m, 1H), 1.25-1.10 (m, 1H)

### Preparation B3-15: N-(1-(2-chloro-5-(1-(tetrahydro-2H-pyran-4-yl)-1H-pyrazol-4-yl)pyridin-4-yl)piperidin-3-yl)acetamide

The titled compound was prepared in accordance with the same procedures as in Preparation B3-1, using the B2-15 intermediate. 1H NMR (CDCl₃, 400 MHz) δ 8.14 (s, 1H), 7.82-7.75 (m, 2H), 6.88 (s, 1H), 5.45-5.35 (m, 1H), 4.50-4.35 (m, 1H), 4.15 (d, 2H), 4.10-4.00 (m, 1H), 3.59 (t, 2H), 3.34 (d, 1H), 3.08 (d, 1H), 2.65 (t, 1H), 2.58 (t, 1H), 2.25-2.10 (m, 5H), 2.10-2.05 (m, 1H), 1.99 (s, 3H), 1.95-1.85 (m, 1H), 1.75-1.65 (m, 1H), 1.45-1.35 (m, 1H)

### Preparation B3-16: 2-chloro-5-(1-(tetrahydro-2H-pyran-4-yl)-1H-pyrazol-4-yl)-4-(4-(trifluoromethyl)piperidin-1-yl)pyridine

The titled compound was prepared in accordance with the same procedures as in Preparation B3-1, using the B2-16 intermediate. 1H NMR (CDCl₃, 400 MHz) δ 8.15 (s, 1H), 7.83 (s, 1H), 7.78 (s, 1H), 6.87 (s, 1H), 4.50-4.35 (m, 1H), 4.15 (d, 2H), 3.59 (t, 2H), 3.43 (d, 2H), 2.67 (t, 2H), 2.20-2.05 (m, 5H), 1.91 (d, 2H), 1.70-1.55 (m, 2H)

### Preparation B3-17: tert-butyl 8-[2-chloro-5-(1-tetrahydropyran-4-ylpyrazol-4-yl)-4-pyridyl]-2,8-diazaspiro[4.5]decan-2-carboxylate

The titled compound was prepared in accordance with the same procedures as in Preparation B3-1, using the B2-17 intermediate. 1H NMR (CDCl₃, 400 MHz) δ 8.12 (s, 1H), 7.86 (d, 1H), 7.76 (s, 1H), 6.87 (d, 1H), 4.45-4.35 (m, 1H), 4.15 (d, 2H), 3.59 (t, 2H), 3.45-3.30 (m, 2H), 3.25 (s, 1H), 3.17 (s, 1H), 3.05-2.95 (m, 2H), 2.95-2.80 (m, 2H), 2.16-2.05 (m, 4H), 1.80-1.70 (m, 2H), 1.65-1.50 (m, 4H), 1.47 (s, 9H)

### Preparation B3-18: tert-butyl 2-[2-chloro-5-(1-tetrahydropyran-4-ylpyrazol-4-yl)-4-pyridyl]-2,8-diazaspiro[4.5]decan-8-carboxylate

The titled compound was prepared in accordance with the same procedures as in Preparation B3-1, using the B2-18 intermediate. 1H NMR (CDCl₃, 400 MHz) δ 7.87 (s, 1H), 7.50 (s, 1H), 7.44 (s, 1H), 6.54 (s, 1H), 4.45-4.35 (m, 1H), 4.14 (d, 2H), 3.58 (t, 2H), 3.45-3.39 (m, 2H), 3.31-3.24 (m, 2H), 3.15 (t, 2H), 2.95 (s, 2H), 2.20-2.00 (m, 4H), 1.73 (t, 2H), 1.46 (s, 9H)

### Preparation B3-19: (3R,4R)-1-(2-chloro-5-(1-(tetrahydro-2H-pyran-4-yl)-1H-pyrazol-4-yl)pyridin-4-yl)-4-fluoropiperidin-3-ol

The titled compound was prepared in accordance with the same procedures as in Preparation B3-1, using the B2-19 intermediate. 1H NMR (CDCl₃, 400 MHz) δ 8.15 (s, 1H), 7.79 (s, 1H), 7.76 (s, 1H), 6.90 (s, 1H), 4.60-4.35 (m, 2H), 4.15 (d, 2H), 3.95-3.80 (m, 1H), 3.58 (t, 2H), 3.45-3.35 (m, 1H), 3.25-3.15 (m, 1H), 2.81-2.70 (m, 2H), 2.25-2.00 (m, 6H), 1.85-1.70 (m, 1H)

### Preparation B3-20: (3R,4R)-1-(2-chloro-5-(1-(tetrahydro-2H-pyran-4-yl)-1H-pyrazol-4-yl)pyridin-4-yl)-4-(hydroxymethyl)piperidin-3-ol

The titled compound was prepared in accordance with the same procedures as in Preparation B3-1, using the B2-20 intermediate. 1H NMR (CDCl₃, 400 MHz) δ 8.13 (s, 1H), 7.81 (s, 1H), 7.72 (s, 1H), 6.89 (s, 1H), 4.45-4.35 (m, 1H), 4.15 (d, 2H), 3.90-3.80 (m, 1H), 3.80-3.65 (m, 2H), 3.58 (t, 2H), 3.48 (d, 1H), 3.32 (d, 1H), 3.19 (s, 1H), 2.59 (t, 1H), 2.45 (t, 1H), 2.25-2.00 (m, 5H), 1.75-1.60 (m, 2H), 1.35-1.20 (m, 1H)

### Preparation B3-21: tert-butyl (R)-(1-(2-chloro-5-(1-(tetrahydro-2H-pyran-4-yl)-1H-pyrazol-4-yl)pyridin-4-yl)pyrrolidin-3-yl)carbamate

The titled compound was prepared in accordance with the same procedures as in Preparation B3-1, using the B2-21 intermediate. 1H NMR (CDCl₃, 400 MHz) δ 7.88 (s, 1H), 7.51 (s, 1H), 7.45 (s, 1H), 6.56 (s, 1H), 4.60-4.45 (m, 1H), 4.45-4.30 (m, 1H), 4.20-4.05 (m, 3H), 3.58 (t, 2H), 3.40-3.30 (m, 1H), 3.30-3.20 (m, 1H), 3.20-3.12 (m, 1H), 2.93-2.88 (m, 1H), 2.20-2.05 (m, 5H), 1.85-1.75 (m, 1H), 1.44 (s, 9H)

### Preparation B3-22: tert-butyl (S)-(1-(2-chloro-5-(1-(tetrahydro-2H-pyran-4-yl)-1H-pyrazol-4-yl)pyridin-4-yl)pyrrolidin-3-yl)carbamate

The titled compound was prepared in accordance with the same procedures as in Preparation B3-1, using the B2-22 intermediate. 1H NMR (CDCl₃, 400 MHz) δ 7.88 (s, 1H), 7.51 (s, 1H), 7.45 (s, 1H), 6.56 (s, 1H), 4.60-4.45 (m, 1H), 4.45-4.30 (m, 1H), 4.20-4.05 (m, 3H), 3.58 (t, 2H), 3.40-3.30 (m, 1H), 3.27-3.20 (m, 1H), 3.20-3.12 (m, 1H), 2.93-2.89 (m, 1H), 2.20-2.05 (m, 5H), 1.85-1.75 (m, 1H), 1.44 (s, 9H)

### Preparation B3-23: 8-(2-chloro-5-(1-(tetrahydro-2H-pyran-4-yl)-1H-pyrazol-4-yl)pyridin-4-yl)-2-methyl-2,8-diazaspiro[4.5]decane

The titled compound was prepared in accordance with the same procedures as in Preparation B3-1, using the B2-23 intermediate. 1H NMR (CDCl₃, 400 MHz) δ 8.11 (s, 1H), 8.08 (s, 1H), 7.89 (s, 1H), 7.02 (s, 1H), 4.55-4.45 (m, 1H), 4.09 (d, 2H), 3.60 (t, 2H), 3.55-3.40 (m, 2H), 3.05-2.95 (m, 2H), 2.70-2.65 (m, 2H), 2.57 (s, 1H), 2.45-2.35 (m, 4H), 2.20-2.00 (m, 4H), 1.85-1.75 (m, 2H), 1.75-1.60 (m, 4H)

### Preparation B3-24: (3S)-1-[2-chloro-5-(1-tetrahydropyran-4-ylpyrazol-4-yl)-4-pyridyl]-N-methyl-pyrrolidin-3-amine

The titled compound was prepared in accordance with the same procedures as in Preparation B3-1, using the B2-24 intermediate. 1H NMR (CD₃OD, 400 MHz) S 7.81 (s, 1H), 7.76 (s, 1H), 7.56 (s, 1H), 6.69 (s, 1H), 4.50-4.40 (m, 1H), 4.08 (d, 2H), 3.59 (t, 2H), 3.35-3.25 (m, 2H), 3.25-3.10 (m, 2H), 2.99-2.94 (m, 1H), 2.39 (s, 3H), 2.20-2.00 (m, 6H), 1.80-1.70 (m, 1H)

### Preparation B3-25: (3S)-1-[2-chloro-5-(1-tetrahydropyran-4-ylpyrazol-4-yl)-4-pyridyl]-N-ethyl-pyrrolidin-3-amine

The titled compound was prepared in accordance with the same procedures as in Preparation B3-1, using the B2-25 intermediate. 1H NMR (CDCl₃, 400 MHz) δ 7.86 (s, 1H), 7.52 (s, 1H), 7.45 (s, 1H), 6.54 (s, 1H), 4.45-4.30 (m, 1H), 4.14 (d, 2H), 3.57 (t, 2H), 3.40-3.30 (m, 1H), 3.30-3.20 (m, 2H), 3.20-3.10 (m, 1H), 2.90-2.80 (m, 1H), 2.70-2.50 (m, 2H), 2.20-2.00 (m, 6H), 1.80-1.65 (m, 1H), 1.10 (t, 3H)

### Preparation B3-26: (R)-N-(1-(2-chloro-5-(1-(tetrahydro-2H-pyran-4-yl)-1H-pyrazol-4-yl)pyridin-4-yl)pyrrolidin-3-yl)acetamide

The titled compound was prepared in accordance with the same procedures as in Preparation B3-1, using the B2-26 intermediate. 1H NMR (CDCl₃, 400 MHz) δ 7.88 (s, 1H), 7.51 (s, 1H), 7.46 (s, 1H), 6.56 (s, 1H), 5.65-5.55 (m, 1H), 4.50-4.35 (m, 2H), 4.14 (d, 2H), 3.58 (t, 2H), 3.40-3.30 (m, 1H), 3.30-3.15 (m, 2H), 2.95-2.85 (m, 1H), 2.20-2.00 (m, 5H), 1.98 (s, 3H), 1.90-1.80 (m, 1H)

### Preparation B3-27: (S)-N-(1-(2-chloro-5-(1-(tetrahydro-2H-pyran-4-yl)-1H-pyrazol-4-yl)pyridin-4-yl)pyrrolidin-3-yl)acetamide

The titled compound was prepared in accordance with the same procedures as in Preparation B3-1, using the B2-27 intermediate. 1H NMR (CDCl₃, 400 MHz) δ 7.87 (s, 1H), 7.51 (s, 1H), 7.46 (s, 1H), 6.56 (s, 1H), 5.65-5.55 (m, 1H), 4.50-4.30 (m, 2H), 4.15 (d, 2H), 3.58 (t, 2H), 3.40-3.30 (m, 1H), 3.30-3.10 (m, 2H), 2.95-2.85 (m, 1H), 2.20-2.00 (m, 5H), 1.97 (s, 3H), 1.90-1.80 (m, 1H)

### Preparation B3-28: 8-(2-chloro-5-(1-(difluoromethyl)-1H-pyrazol-4-yl)pyridin-4-yl)-2-methyl-2,8-diazaspiro[4.5]decane

The titled compound was prepared in accordance with the same procedures as in Preparation B3-1, using the B2-23 intermediate and changing the boronic acid reagent to 1-(difluoromethyl)pyrazol-4-boronic acid pinacol ester. 1H NMR (CDCl₃, 400 MHz) δ 8.17 (s, 1H), 8.15 (s, 1H), 8.03 (s, 1H), 7.45-7.05 (m, 1H), 6.91 (s, 1H), 2.93 (t, 4H), 2.57 (t, 2H), 2.41 (s, 2H), 2.32 (s, 3H), 1.71 (t, 4H), 1.69-1.62 (m, 2H)

### Preparation B3-29: 8-(2-chloro-5-(1-(2,2,2-trifluoroethyl)-1H-pyrazol-4-yl)pyridin-4-yl)-2-methyl-2,8-diazaspiro[4.5]decane

The titled compound was prepared in accordance with the same procedures as in Preparation B3-1, using the B2-23 intermediate and changing the boronic acid reagent to 4-(4,4,5,5-tetramethyl-1,3,2-dioxaborolan-2-yl)-1-(2,2,2-trifluoroethyl)pyrazole. 1H NMR (CDCl₃, 400 MHz) δ 8.12 (s, 1H), 7.93 (s, 1H), 7.87 (s, 1H), 6.89 (s, 1H), 4.78 (q, 2H), 2.93 (t, 4H), 2.60 (t, 2H), 2.43 (s, 2H), 2.36 (s, 3H), 1.70 (t, 2H), 1.70-1.60 (m, 4H)

### Preparation B3-30: 4-(6′-chloro-4′-(2-methyl-2,8-diazaspiro[4.5]decan-8-yl)-[3,3′-bipyridin]-6-yl)morpholine

The titled compound was prepared in accordance with the same procedures as in Preparation B3-1, using the B2-23 intermediate and changing the boronic acid reagent to 6-(morpholin-4-yl)pyridin-3-boronic acid pinacol ester. 1H NMR (CDCl₃, 400 MHz) δ 8.35 (s, 1H), 7.99 (s, 1H), 7.76 (d, 1H), 6.85 (s, 1H), 6.72 (d, 1H), 3.88 (t, 4H), 3.58 (t, 4H), 2.94 (t, 4H), 2.57 (t, 2H), 2.44 (s, 2H), 2.33 (s, 3H), 1.65 (t, 2H), 1.65-1.45 (m, 4H)

### Preparation B3-31: 2-(1-(2-chloro-5-(1-(difluoromethyl)-1H-pyrazol-4-yl)pyridin-4-yl)piperidin-4-yl)-N,N-dimethylethan-1-amine

The titled compound was prepared in accordance with the same procedures as in Preparation B3-1, using the B2-8 intermediate and changing the boronic acid reagent to 1-(difluoromethyl)pyrazol-4-boronic acid pinacol ester. 1H NMR (CDCl₃, 400 MHz) δ 8.16 (s, 1H), 8.15 (s, 1H), 8.02 (s, 1H), 7.45-7.05 (m, 1H), 6.89 (s, 1H), 3.29 (d, 2H), 2.62 (t, 2H), 2.31 (t, 2H), 2.26 (s, 1H), 2.24 (s, 6H), 1.75 (d, 2H), 1.48 (t, 2H), 1.35-1.20 (m, 2H)

### Preparation B3-32: 2-(1-(2-chloro-5-(1-(2,2,2-trifluoroethyl)-1H-pyrazol-4-yl)pyridin-4-yl)piperidin-4-yl)-N,N-dimethylethan-1-amine

The titled compound was prepared in accordance with the same procedures as in Preparation B3-1, using the B2-8 intermediate and changing the boronic acid reagent to 4-(4,4,5,5-tetramethyl-1,3,2-dioxaborolan-2-yl)-1-(2,2,2-trifluoroethyl)pyrazole. 1H NMR (CDCl₃, 400 MHz) δ 8.12 (s, 1H), 7.92 (s, 1H), 7.87 (s, 1H), 6.88 (s, 1H), 4.78 (q, 2H), 3.31 (d, 2H), 2.59 (t, 2H), 2.31 (t, 2H), 2.27 (s, 1H), 2.25 (s, 6H), 1.73 (d, 2H), 1.47 (t, 2H), 1.30-1.20 (m, 2H)

### Preparation B3-33: 2-(1-(6-chloro-6′-morpholino-[3,3′-bipyridin]-4-yl)piperidin-4-yl)-N,N-dimethylethan-1-amine

The titled compound was prepared in accordance with the same procedures as in Preparation B3-1, using the B2-8 intermediate and changing the boronic acid reagent to 6-(morpholin-4-yl)pyridin-3-boronic acid pinacol ester. 1H NMR (CDCl₃, 400 MHz) δ 8.34 (s, 1H), 7.98 (s, 1H), 7.76 (d, 1H), 6.84 (s, 1H), 6.72 (d, 1H), 3.88 (t, 4H), 3.59 (t, 4H), 3.34 (d, 2H), 2.59 (t, 2H), 2.30-2.25 (m, 3H), 2.23 (s, 6H), 1.66 (d, 2H), 1.43 (t, 2H), 1.25-1.10 (m, 2H)

### Preparation B3-34: tert-butyl (R)-(1-(2-chloro-5-(1-(2,2,2-trifluoroethyl)-1H-pyrazol-4-yl)pyridin-4-yl)piperidin-3-yl)carbamate

The titled compound was prepared in accordance with the same procedures as in Preparation B3-1, using the B2-7 intermediate and changing the boronic acid reagent to 4-(4,4,5,5-tetramethyl-1,3,2-dioxaborolan-2-yl)-1-(2,2,2-trifluoroethyl)pyrazole. 1H NMR (CDCl₃, 400 MHz) δ 8.15 (s, 1H), 7.88 (s, 1H), 7.82 (s, 1H), 6.88 (s, 1H), 4.83-4.80 (m, 2H), 4.47-4.45 (m, 1H), 3.76-3.68 (m, 1H), 3.44-3.41 (m, 1H), 3.12-3.09 (m, 1H), 2.63-2.58 (m, 1H), 2.43-2.41 (m, 1H), 1.97-1.94 (m, 1H), 1.75-1.69 (m, 1H), 1.44 (s, 10H)

### Preparation B3-35: (1S,4S)-4-((2-chloro-5-(1-(difluoromethyl)-1H-pyrazol-4-yl)pyridin-4-yl)(methyl)amino)cyclohexan-1-ol

The titled compound was prepared in accordance with the same procedures as in Preparation B3-1, using the B2-29 intermediate and changing the boronic acid reagent to 1-(difluoromethyl)pyrazol-4-boronic acid pinacol ester. 1H NMR (CDCl₃, 400 MHz) δ 8.04 (d, 2H), 7.86 (s, 1H), 7.26 (t, 1H), 6.86 (s, 1H), 3.96 (brs, 1H), 3.22-3.14 (m, 1H), 2.72 (s, 3H), 1.98-1.88 (m, 2H), 1.80 (d, 2H), 1.36-1.25 (m, 5H)

### Preparation B3-36: tert-butyl N-[(3S,6R)-1-[2-chloro-5-[1-(difluoromethyl)pyrazol-4-yl]-4-pyridyl]-6-methyl-3-piperidyl]carbamate

The titled compound was prepared in accordance with the same procedures as in Preparation B3-1, using the B2-30 intermediate and changing the boronic acid reagent to 1-(difluoromethyl)pyrazol-4-boronic acid pinacol ester. 1H NMR (CDCl₃, 400 MHz) δ 8.19 (s, 2H), 8.00 (s, 1H), 7.42-7.38 (m, 0.2H), 7.27-7.22 (m, 0.5H), 7.12-7.07 (m, 0.3H), 6.92-6.82 (m, 1H), 4.59-4.44 (m, 1H), 3.83-3.63 (m, 1H), 3.59-3.43 (m, 1H), 3.38-3.23 (m, 1H), 2.89-2.75 (m, 1H), 1.93-1.82 (m, 1H), 1.82-1.68 (m, 1H), 1.53 (s, 9H), 1.33-1.26 (m, 1H), 0.95 (d, 3H)

### Preparation B3-37: tert-butyl N-[(3R,4S)-1-[2-chloro-5-[1-(difluoromethyl)pyrazol-4-yl]-4-pyridyl]-4-methoxy-3-piperidyl]carbamate

The titled compound was prepared in accordance with the same procedures as in Preparation B3-1, using the B2-31 intermediate and changing the boronic acid reagent to 1-(difluoromethyl)pyrazol-4-boronic acid pinacol ester. 1H NMR (CDCl₃, 400 MHz) δ 8.18-8.09 (m, 2H), 7.99 (s, 1H), 7.44-7.40 (m, 0.2H), 7.28-7.26 (m, 0.6H), 7.13-7.11 (m, 0.2H), 6.97 (s, 1H), 5.12-5.01 (m, 1H), 3.91-3.76 (m, 1H), 3.59-3.51 (m, 1H), 3.41 (s, 3H), 3.25-3.14 (m, 1H), 3.00-2.85 (m, 2H), 2.85-2.74 (m, 1H), 2.01-1.89 (m, 1H), 1.52 (s, 9H)

### Preparation B3-38: tert-butyl N-[(3R,4R)-1-[2-chloro-5-[1-(difluoromethyl)pyrazol-4-yl]-4-pyridyl]-4-methoxy-3-piperidyl]carbamate

The titled compound was prepared in accordance with the same procedures as in Preparation B3-1, using the B2-32 intermediate and changing the boronic acid reagent to 1-(difluoromethyl)pyrazol-4-boronic acid pinacol ester. 1H NMR (CDCl₃, 400 MHz) δ 8.15 (s, 1H), 8.10 (s, 1H), 7.94 (s, 1H), 7.41-7.39 (m, 0.2H), 7.26-7.24 (m, 0.6H), 7.11-7.00 (m, 0.2H), 4.73-4.69 (m, 1H), 3.71-3.69 (m, 1H), 3.67-3.58 (m, 1H), 3.40 (s, 3H), 3.29-3.22 (m, 1H), 3.11-3.08 (m, 2H), 2.76-2.72 (m, 1H), 1.98-1.96 (m, 1H), 1.26 (s, 9H)

### Preparation B3-39: tert-butyl (R)-((1-(2-chloro-5-(1-(difluoromethyl)-1H-pyrazol-4-yl)pyridin-4-yl)piperidin-3-yl)methyl)(methyl)carbamate

The titled compound was prepared in accordance with the same procedures as in Preparation B3-1, using the B2-33 intermediate and changing the boronic acid reagent to 1-(difluoromethyl)pyrazol-4-boronic acid pinacol ester. 1H NMR (CDCl₃, 400 MHz) δ 8.20-8.05 (m, 2H), 8.00 (s, 1H), 7.45-7.10 (m, 1H), 6.89 (s, 1H), 3.30-3.10 (m, 3H), 3.10-2.90 (m, 1H), 2.80-2.70 (m, 3H), 2.59 (t, 1H), 2.45-2.25 (m, 1H), 2.00-1.85 (m, 1H), 1.85-1.70 (m, 2H), 1.65-1.50 (m, 1H), 1.48 (s, 9H), 1.15-1.00 (m, 1H)

### Preparation B3-40: tert-butyl (S)-((1-(2-chloro-5-(1-(difluoromethyl)-1H-pyrazol-4-yl)pyridin-4-yl)piperidin-3-yl)methyl)(methyl)carbamate

The titled compound was prepared in accordance with the same procedures as in Preparation B3-1, using the B2-34 intermediate and changing the boronic acid reagent to 1-(difluoromethyl)pyrazol-4-boronic acid pinacol ester. 1H NMR (CDCl₃, 400 MHz) δ 8.20-8.05 (m, 2H), 8.00 (s, 1H), 7.45-7.10 (m, 1H), 6.89 (s, 1H), 3.30-3.05 (m, 3H), 3.05-2.90 (m, 1H), 2.85-2.70 (m, 3H), 2.59 (t, 1H), 2.45-2.25 (m, 1H), 2.00-1.80 (m, 1H), 1.80-1.70 (m, 2H), 1.65-1.55 (m, 1H), 1.45 (s, 9H), 1.20-1.00 (m, 1H)

### Preparation B3-41: tert-butyl ((3R,4R)-1-(2-chloro-5-(1-(difluoromethyl)-1H-pyrazol-4-yl)pyridin-4-yl)-3-methylpiperidin-4-yl)carbamate

The titled compound was prepared in accordance with the same procedures as in Preparation B3-1, using the B2-35 intermediate and changing the boronic acid reagent to 1-(difluoromethyl)pyrazol-4-boronic acid pinacol ester. 1H NMR (CDCl₃, 400 MHz) δ 8.20-8.10 (m, 2H), 7.98 (s, 1H), 7.45-7.10 (m, 1H), 7.11 (s, 1H), 4.45-4.35 (m, 1H), 3.35-3.20 (m, 3H), 2.69 (t, 1H), 2.40 (t, 1H), 2.05-1.95 (m, 1H), 1.55-1.50 (m, 1H), 1.47 (s, 9H), 0.93 (d, 3H)

### Preparation B3-42: 2-chloro-5-[1-(difluoromethyl)pyrazol-4-yl]-4-[(3S)-3-methyl-1-piperidyl]pyridine

The titled compound was prepared in accordance with the same procedures as in Preparation B3-1, using the B2-36 intermediate and changing the boronic acid reagent to 1-(difluoromethyl)pyrazol-4-boronic acid pinacol ester. 1H NMR (CDCl₃, 400 MHz) δ 8.17 (s, 1H), 8.15 (s, 1H), 7.99 (s, 1H), 7.45-7.10 (m, 1H), 6.90 (s, 1H), 3.30-3.15 (m, 2H), 2.60-2.50 (m, 1H), 2.30-2.20 (m, 1H), 1.90-1.80 (m, 1H), 1.80-1.65 (m, 2H), 1.65-1.50 (m, 1H), 1.10-1.00 (m, 1H), 0.87 (d, 3H)

### Preparation B3-43: 2-chloro-5-[1-(difluoromethyl)pyrazol-4-yl]-4-[(3S)-3-(fluoromethyl)-1-piperidyl]pyridine

The titled compound was prepared in accordance with the same procedures as in Preparation B3-1, using the B2-37 intermediate and changing the boronic acid reagent to 1-(difluoromethyl)pyrazol-4-boronic acid pinacol ester. 1H NMR (CDCl₃, 400 MHz) δ 8.16 (s, 1H), 8.15 (s, 1H), 7.98 (s, 1H), 7.45-7.10 (m, 1H), 6.93 (s, 1H), 4.45-4.10 (m, 2H), 3.45-3.35 (m, 1H), 3.30-3.15 (m, 1H), 2.59 (t, 1H), 2.52 (t, 1H), 2.15-2.00 (m, 1H), 1.85-1.70 (m, 2H), 1.70-1.55 (m, 1H), 1.30-1.10 (m, 1H)

### Preparation B3-44: 2-chloro-5-[1-(difluoromethyl)pyrazol-4-yl]-4-[(3R)-3-(difluoromethyl)-1-piperidyl]pyridine

The titled compound was prepared in accordance with the same procedures as in Preparation B3-1, using the B2-38 intermediate and changing the boronic acid reagent to 1-(difluoromethyl)pyrazol-4-boronic acid pinacol ester. 1H NMR (CDCl₃, 400 MHz) δ 8.18 (s, 1H), 8.14 (s, 1H), 7.97 (s, 1H), 7.45-7.10 (m, 1H), 6.93 (s, 1H), 5.80-5.45 (m, 1H), 3.45-3.35 (m, 1H), 3.30-3.20 (m, 1H), 2.65-2.55 (m, 2H), 2.20-2.00 (m, 1H), 2.00-1.90 (m, 1H), 1.85-1.75 (m, 1H), 1.65-1.50 (m, 1H), 1.45-1.30 (m, 1H)

### Preparation B3-45: tert-butyl N-[(3S,4S)-1-[2-chloro-5-[1-(difluoromethyl)pyrazol-4-yl]-4-pyridyl]-3-fluoro-4-piperidyl]carbamate

The titled compound was prepared in accordance with the same procedures as in Preparation B3-1, using the B2-39 intermediate and changing the boronic acid reagent to 1-(difluoromethyl)pyrazol-4-boronic acid pinacol ester. 1H NMR (CDCl₃, 400 MHz) δ 7.95 (s, 1H), 7.94 (s, 1H), 7.78 (s, 1H), 7.43-7.12 (m, 1H), 6.70 (s, 1H), 4.45-4.35 (m, 1H), 4.35-4.20 (m, 1H), 4.05-3.90 (m, 1H), 3.70-3.55 (m, 1H), 3.15-3.00 (m, 1H), 3.00-2.95 (m, 1H), 2.20-2.10 (m, 1H), 1.46 (s, 9H), 1.45-1.35 (m, 1H)

### Preparation B3-46: tert-butyl N-[(3R,4S)-1-[2-chloro-5-[1-(difluoromethyl)pyrazol-4-yl]-4-pyridyl]-3-fluoro-4-piperidyl]carbamate

The titled compound was prepared in accordance with the same procedures as in Preparation B3-1, using the B2-40 intermediate and changing the boronic acid reagent to 1-(difluoromethyl)pyrazol-4-boronic acid pinacol ester. 1H NMR (CDCl₃, 400 MHz) δ 8.25 (s, 1H), 8.21 (s, 1H), 8.00 (s, 1H), 7.41-7.10 (m, 1H), 6.93 (s, 1H), 4.95-4.85 (m, 1H), 4.85-4.70 (m, 1H), 3.87-3.70 (m, 1H), 3.70-3.60 (m, 1H), 3.35-3.25 (m, 1H), 3.00-2.80 (m, 1H), 2.80-2.70 (m, 1H), 1.90-1.80 (m, 1H), 1.48 (s, 9H)

### Preparation B3-47: tert-butyl N-[(3S,4R)-1-[2-chloro-5-[1-(difluoromethyl)pyrazol-4-yl]-4-pyridyl]-3-fluoro-4-piperidyl]carbamate

The titled compound was prepared in accordance with the same procedures as in Preparation B3-1, using the B2-41 intermediate and changing the boronic acid reagent to 1-(difluoromethyl)pyrazol-4-boronic acid pinacol ester. 1H NMR (CDCl₃, 400 MHz) δ 8.25 (s, 1H), 8.21 (s, 1H), 8.07 (s, 1H), 7.41-7.02 (m, 1H), 6.93 (s, 1H), 4.95-4.85 (m, 1H), 4.85-4.70 (m, 1H), 3.85-3.70 (m, 1H), 3.70-3.60 (m, 1H), 3.40-3.25 (m, 1H), 3.00-2.80 (m, 1H), 2.80-2.70 (m, 1H), 1.85-1.75 (m, 1H), 1.48 (s, 9H)

### Preparation B3-48: tert-butyl N-[(3S,5R)-1-[2-chloro-5-[1-(difluoromethyl)pyrazol-4-yl]-4-pyridyl]-5-fluoro-3-piperidyl]carbamate

The titled compound was prepared in accordance with the same procedures as in Preparation B3-1, using the B2-42 intermediate and changing the boronic acid reagent to 1-(difluoromethyl)pyrazol-4-boronic acid pinacol ester. 1H NMR (CDCl₃, 400 MHz) δ 8.23 (s, 1H), 8.15 (s, 1H), 7.94 (s, 1H), 7.43-7.39 (m, 0.2H), 7.28-7.25 (m, 0.6H), 7.13-7.10 (m, 0.2H), 7.03 (s, 1H), 5.12-4.97 (m, 1H), 4.84-4.74 (m, 0.5H), 4.72-4.62 (m, 0.5H), 4.03-3.87 (m, 1H), 3.31-3.19 (m, 1H), 3.19-3.10 (m, 2H), 3.07-2.91 (m, 1H), 2.20-2.10 (m, 0.5H), 2.10-2.03 (m, 0.5H), 2.03-1.88 (m, 1H), 1.51 (s, 9H)

### Preparation B3-49: tert-butyl N-[(3S,5S)-1-[2-chloro-5-[1-(difluoromethyl)pyrazol-4-yl]-4-pyridyl]-5-fluoro-3-piperidyl]carbamate

The titled compound was prepared in accordance with the same procedures as in Preparation B3-1, using the B2-43 intermediate and changing the boronic acid reagent to 1-(difluoromethyl)pyrazol-4-boronic acid pinacol ester. 1H NMR (CDCl₃, 400 MHz) δ 8.25 (s, 2H), 7.97 (s, 1H), 7.44-7.40 (m, 0.2H), 7.27-7.25 (m, 0.6H), 7.13-7.09 (m, 0.2H), 7.02 (s, 1H), 4.95-4.86 (m, 0.5H), 4.83-4.75 (m, 0.5H), 4.47-4.32 (m, 1H), 4.12-3.98 (m, 1H), 3.63-3.53 (m, 1H), 3.53-3.43 (m, 1H), 2.84-2.76 (m, 0.5H), 2.76-2.68 (m, 0.5H), 2.60-2.49 (m, 1H), 2.42-2.29 (m, 1H), 1.50 (s, 9H)

### Preparation B3-50: tert-butyl N-[(3S,5R)-1-[2-chloro-5-[1-(difluoromethyl)pyrazol-4-yl]-4-pyridyl]-5-(trifluoromethyl)-3-piperidyl]carbamate

The titled compound was prepared in accordance with the same procedures as in Preparation B3-1, using the B2-44 intermediate and changing the boronic acid reagent to 1-(difluoromethyl)pyrazol-4-boronic acid pinacol ester. 1H NMR (CDCl₃, 400 MHz) 8.64 (s, 1H), 8.12 (s, 1H), 7.99 (s, 1H), 7.28-7.26 (m, 0.2H), 7.26-7.20 (m, 0.6H), 7.02-7.01 (m, 0.2H), 6.93-6.93 (m, 1H), 4.43-4.41 (m, 1H), 3.73-3.71 (m, 2H), 3.63-3.60 (m, 1H), 3.45-3.42 (m, 1H), 2.66-2.52 (m, 1H), 2.49-2.33 (m, 2H), 1.47 (s, 9H)

### Preparation B3-51: tert-butyl N-[(3S,5S)-1-[2-chloro-5-[1-(difluoromethyl)pyrazol-4-yl]-4-pyridyl]-5-hydroxy-3-piperidyl]carbamate

The titled compound was prepared in accordance with the same procedures as in Preparation B3-1, using the B2-45 intermediate and changing the boronic acid reagent to 1-(difluoromethyl)pyrazol-4-boronic acid pinacol ester. 1H NMR (CDCl₃, 400 MHz) δ 8.17-8.15 (m, 2H), 7.93 (s, 1H), 7.41-7.40 (m, 0.2H), 7.28-7.20 (m, 0.6H), 7.11-7.09 (m, 0.2H), 6.98 (s, 1H), 5.15-5.10 (m, 1H), 3.92-3.90 (m, 1H), 3.89-3.82 (m, 1H), 3.33-3.30 (m, 1H), 3.12-3.09 (m, 1H), 2.81-2.76 (m, 2H), 2.16-2.13 (m, 1H), 1.55-1.52 (m, 1H), 1.45 (s, 9H)

### Preparation B3-52: tert-butyl N-[(3R,4R)-1-[2-chloro-5-[1-(difluoromethyl)pyrazol-4-yl]-4-pyridyl]-3-hydroxy-4-piperidyl]carbamate

The titled compound was prepared in accordance with the same procedures as in Preparation B3-1, using the B2-46 intermediate and changing the boronic acid reagent to 1-(difluoromethyl)pyrazol-4-boronic acid pinacol ester. 1H NMR (CDCl₃, 400 MHz) δ 8.17 (s, 1H), 8.11 (s, 1H), 7.96 (s, 1H), 7.41-7.10 (m, 1H), 6.95 (s, 1H), 4.75-4.65 (m, 1H), 4.10-4.00 (m, 1H), 3.60-3.40 (m, 2H), 3.30-3.20 (m, 1H), 2.75-2.60 (m, 1H), 2.60-2.55 (m, 1H), 2.00-1.90 (m, 1H), 1.48 (s, 9H)

### Preparation B3-53: (3R,4R)-1-[2-chloro-5-[1-(difluoromethyl)pyrazol-4-yl]-4-pyridyl]-4-fluoro-piperidin-3-ol

The titled compound was prepared in accordance with the same procedures as in Preparation B3-1, using the B2-19 intermediate and changing the boronic acid reagent to 1-(difluoromethyl)pyrazol-4-boronic acid pinacol ester. 1H NMR (CDCl₃, 400 MHz) δ 8.20 (s, 1H), 8.18 (s, 1H), 7.98 (s, 1H), 7.42-7.12 (m, 1H), 6.96 (s, 1H), 4.60-4.40 (m, 1H), 3.95-3.85 (m, 1H), 3.45-3.35 (m, 1H), 3.25-3.15 (m, 1H), 2.85-2.75 (m, 1H), 2.30-2.20 (m, 1H), 2.20-2.10 (m, 1H), 1.90-1.75 (m, 1H)

### Preparation B3-54: tert-butyl ((3S,5R)-1-(2-chloro-5-(1-(2,2,2-trifluoroethyl)-1H-pyrazol-4-yl)pyridin-4-yl)-5-fluoropiperidin-3-yl)carbamate

The titled compound was prepared in accordance with the same procedures as in Preparation B3-1, using the B2-42 intermediate and changing the boronic acid reagent to 4-(4,4,5,5-tetramethyl-1,3,2-dioxaborolan-2-yl)-1-(2,2,2-trifluoroethyl)pyrazole. 1H NMR (CDCl₃, 400 MHz) δ 8.24 (s, 1H), 7.91 (s, 1H), 7.84 (s, 1H), 7.02 (s, 1H), 5.03-4.93 (m, 1H), 4.87-4.75 (m, 3H), 3.99-3.87 (m, 1H), 3.27-3.11 (m, 2H), 3.11-2.95 (m, 2H), 2.21-2.04 (m, 1H), 1.94-1.84 (m, 1H), 1.50 (s, 9H)

### Preparation B3-55: tert-butyl ((3S,5S)-1-(2-chloro-5-(1-(2,2,2-trifluoroethyl)-1H-pyrazol-4-yl)pyridin-4-yl)-5-fluoropiperidin-3-yl)carbamate

The titled compound was prepared in accordance with the same procedures as in Preparation B3-1, using the B2-43 intermediate and changing the boronic acid reagent to 4-(4,4,5,5-tetramethyl-1,3,2-dioxaborolan-2-yl)-1-(2,2,2-trifluoroethyl)pyrazole. 1H NMR (CDCl₃, 400 MHz) δ 8.24 (s, 1H), 8.03 (s, 1H), 7.84 (s, 1H), 6.97 (s, 1H), 4.91-4.70 (m, 4H), 4.47-4.32 (m, 1H), 4.13-3.98 (m, 1H), 3.61-3.40 (m, 2H), 2.88-2.69 (m, 1H), 2.51-2.29 (m, 2H), 1.51 (s, 9H)

### Preparation B3-56: tert-butyl N-[(3S,5R)-1-[2-chloro-5-(1-isopropylpyrazol-4-yl)-4-pyridyl]-5-fluoro-3-piperidyl]carbamate

The titled compound was prepared in accordance with the same procedures as in Preparation B3-1, using the B2-42 intermediate and changing the boronic acid reagent to 1-isopropyl-4-(4,4,5,5-tetramethyl-1,3,2-dioxaborolan-2-yl)pyrazole. 1H NMR (CDCl₃, 400 MHz) δ 8.25 (s, 1H), 7.83 (s, 1H), 7.75 (s, 1H), 6.98 (s, 1H), 5.14-4.99 (m, 1H), 4.81-4.72 (m, 0.5H), 4.69-4.63 (m, 0.5H), 4.63-4.51 (m, 1H), 4.05-3.92 (m, 1H), 3.33-3.23 (m, 1H), 3.23-3.14 (m, 1H), 3.14-3.05 (m, 1H), 3.01-2.80 (m, 1H), 2.19-2.06 (m, 0.5H), 2.06-1.89 (m, 1.5H), 1.60 (d, 6H), 1.51 (s, 9H)

### Preparation B3-57: tert-butyl N-[(3S,5S)-1-[2-chloro-5-(1-isopropylpyrazol-4-yl)-4-pyridyl]-5-fluoro-3-piperidyl]carbamate

The titled compound was prepared in accordance with the same procedures as in Preparation B3-1, using the B2-43 intermediate and changing the boronic acid reagent to 1-isopropyl-4-(4,4,5,5-tetramethyl-1,3,2-dioxaborolan-2-yl)pyrazole. 1H NMR (CDCl₃, 400 MHz) δ 8.23 (s, 1H), 7.94 (s, 1H), 7.74 (s, 1H), 6.94 (s, 1H), 4.90-4.81 (m, 1H), 4.77-4.70 (m, 1H), 4.64-4.58 (m, 1H), 4.58-4.47 (m, 1H), 4.15-4.01 (m, 1H), 3.58-3.39 (m, 2H), 2.79-2.59 (m, 1H), 2.59-2.48 (m, 1H), 2.39-2.26 (m, 1H), 1.57 (d, 6H), 1.48 (s, 9H)

### Preparation B3-58: tert-butyl N-[(3R,4R)-1-[2-chloro-5-(1-isopropylpyrazol-4-yl)-4-pyridyl]-4-fluoro-3-piperidyl]carbamate

The titled compound was prepared in accordance with the same procedures as in Preparation B3-1, using the B2-47 intermediate and changing the boronic acid reagent to 1-isopropyl-4-(4,4,5,5-tetramethyl-1,3,2-dioxaborolan-2-yl)pyrazole. 1H NMR (CDCl₃, 400 MHz) δ 8.16 (s, 1H), 7.76 (s, 1H), 7.73 (s, 1H), 6.90 (s, 1H), 4.80-4.70 (m, 1H), 4.65-4.55 (m, 1H), 4.55-4.40 (m, 1H), 4.00-3.85 (m, 1H), 3.55-3.45 (m, 1H), 3.10-3.00 (m, 1H), 2.85-2.75 (m, 1H), 2.05-1.95 (m, 1H), 1.85-1.75 (m, 1H), 1.59 (d, 6H), 1.47 (s, 9H)

### Preparation B4-1: 2-chloro-4-fluoro-5-((tetrahydro-2H-pyran-4-yl)ethynyl)pyridine

2-Chloro-4-fluoro-5-iodopyridine (1.0 g, 3.88 mmol) was dissolved in anhydrous 1,4-dioxane (10.00 mL) and then 4-ethynyl-tetrahydro-2H-pyrane (470.7 mg, 4.27 mmol), bis(triphenylphosphine)palladium(II) dichloride (129.8 mg, 0.18 mmol), copper(I) iodide (44.4 mg, 0.23 mmol), and triethylamine (1.0 mL, 7.77 mmol) were added dropwise thereto. Then, the mixed solution was stirred at 80 °C for 4 hours. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (799.6 mg). (Yield: 85.9%) 1H NMR (CDCl₃, 400 MHz) δ 8.41 (d, 1H), 7.11 (d, 1H), 3.95-3.92 (m, 2H), 3.58-3.53 (m, 2H), 2.93-2.89 (m, 1H), 1.95-1.91 (m, 2H), 1.81-1.74 (m, 2H)

### Preparation B4-2: 2-chloro-4-fluoro-5-((1-(trifluoromethyl)-1H-pyrazol-4-yl)ethynyl)pyridine

The titled compound was prepared in accordance with the same procedures as in Preparation B4-1, changing the ethyne reagent to 4-ethynyl-1-(trifluoromethyl)-1H-pyrazole. 1H NMR (CDCl₃, 400 MHz) δ 8.52 (d, 1H), 8.07 (s, 1H), 7.92 (s, 1H), 7.18 (d, 1H)

### Preparation B5-1: 1-(2-chloro-5-((tetrahydro-2H-pyran-4-yl)ethynyl)pyridin-4-yl) azetidin-3-ol

2-Chloro-4-fluoro-5-(2-tetrahydropyran-4-ylethynyl)pyridine (the B4-1 intermediate, 100 mg, 0.42 mmol) was dissolved in anhydrous dimethylformamide (2.0 mL) and then 3-hydroxyazetidine hydrochloride (50.3 mg, 0.46 mmol) and diisopropylethylamine (0.17 mL, 1.25 mmol) were added dropwise thereto. Then, the mixed solution was stirred at room temperature overnight. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (83.3 mg). (Yield: 68.2%) 1H NMR (CDCl₃, 400 MHz) δ 7.99 (s, 1H), 6.16 (s, 1H), 4.77-4.75 (m, 1H), 4.53-4.49 (m, 2H), 4.09-4.08 (m, 2H), 4.06-3.91 (m, 2H), 3.55-3.49 (m, 2H), 2.93-2.81 (m, 1H), 2.51-2.49 (m, 1H), 1.91-1.87 (m, 2H), 1.79-1.72 (m, 2H)

### Preparation B5-2: (R)-1-(2-chloro-5-((1-(trifluoromethyl)-1H-pyrazol-4-yl)ethynyl)pyridin-4-yl)piperidin-3-ol

The titled compound was prepared in accordance with the same procedures as in Preparation B5-1, using the B4-2 intermediate and changing the amine reagent to (R)-piperidin-3-ol. 1H NMR (CDCl₃, 400 MHz) δ 8.27 (s, 1H), 8.16 (s, 1H), 7.92 (s, 1H), 6.83 (s, 1H), 4.01-4.13 (m, 1H), 3.60-3.71 (m, 1H), 3.46-3.20 (m, 2H), 3.17-3.15 (m, 1H), 1.98-1.81 (m, 2H), 1.61-1.56 (m, 2H)

### Preparation C1-1: 2-(2-fluoro-6-methoxyphenyl)pyrimidin-4-amine

2-bromo-4-pyrimidinamine (3.0 g, 17.24 mmol) was dissolved in anhydrous acetonitrile (86.2 mL) and then 2-fluoro-6-methoxyphenylboronic acid (4.4 g, 25.86 mmol), bis(triphenylphosphine)palladium(II) dichloride (605.0 mg, 0.86 mmol), and 2.0M sodium carbonate solution (25.8 mL, 51.72 mmol) were added dropwise thereto. Then, the mixed solution was stirred at 100 °C overnight. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (1.2 g). (Yield: 31.7%) 1H NMR (CDCl₃, 400 MHz) δ 8.39 (d, 1H), 7.34-7.28 (m, 1H), 6.81-6.76 (m, 2H), 6.43 (d, 1H), 4.97 (brs, 2H), 3.81 (s, 3H)

### Preparation C1-2: 5-fluoro-6-(2-fluoro-6-methoxyphenyl)pyridin-2-amine

The titled compound was prepared in accordance with the same procedures as in Preparation C1-1, changing the amine reagent to 6-chloro-5-fluoropyridin-2-amine. 1H NMR (CDCl₃, 400 MHz) δ 7.37-7.30 (m, 2H), 6.82-6.77 (m, 2H), 6.54-6.51 (m, 1H), 4.41 (brs, 2H), 3.81 (s, 3H)

### Preparation C1-3: 5-chloro-6-(2-fluoro-6-methoxyphenyl)pyridin-2-amine

The titled compound was prepared in accordance with the same procedures as in Preparation C1-1, changing the amine reagent to 6-bromo-5-chloro-pyridin-2-amine. 1H NMR (CDCl₃, 400 MHz) δ 7.52 (d, 1H), 7.37-7.33 (m, 1H), 6.82-6.77 (m, 2H), 6.50 (d, 1H), 4.52 (brs, 2H), 3.81 (s, 3H)

### Preparation C1-4: 6-(2-fluoro-6-methoxyphenyl)-5-methylpyridin-2-amine

The titled compound was prepared in accordance with the same procedures as in Preparation C1-1, changing the amine reagent to 6-bromo-5-methyl-pyridin-2-amine. 1H NMR (CDCl₃, 400 MHz) δ 7.37 (d, 1H), 7.35-7.31 (m, 1H), 6.81-6.76 (m, 2H), 6.49 (d, 1H), 4.35 (brs, 2H), 3.78 (s, 3H), 1.99 (s, 3H)

### Preparation C1-5: 6-(2-fluoro-6-methoxy-phenyl)-5-methylsulfonyl-pyridin-2-amine

The titled compound was prepared in accordance with the same procedures as in Preparation C1-1, changing the amine reagent to 6-bromo-5-(methylsulfonyl)pyridin-2-amine. 1H NMR (CDCl₃, 400 MHz) 8.17 (d, 1H), 7.40 (q, 1H), 6.85-6.79 (m, 2H), 6.61 (d, 1H), 5.06 (s, 2H), 3.81 (s, 3H), 2.84 (s, 3H)

### Preparation C1-6: 2-(7-fluoroquinoxalin-6-yl)pyrimidin-4-amine

The titled compound was prepared in accordance with the same procedures as in Preparation C1-1, changing the boronic acid reagent to (7-fluoroquinoxalin-6-yl)boronic acid. 1H NMR (CD₃OD, 400 MHz) 8.94-9.92 (m, 2H), 8.54 (d, 1H), 8.24 (d, 1H), 7.89 (d, 1H), 6.59 (d, 1H)

### Preparation C1-7: 2-(2,4-difluoro-6-methoxyphenyl)pyrimidin-4-amine

The titled compound was prepared in accordance with the same procedures as in Preparation C1-1, changing the boronic acid reagent to (2,4-difluoro-6-methoxy-phenyl)boronic acid. 1H NMR (CD₃OD, 400 MHz) 8.38 (d, 1H), 6.53-6.50 (m, 2H), 6.43 (d, 1H), 5.00 (brs, 2H), 3.78 (s, 3H)

### Preparation C1-8: 6-(2-fluoro-6-methoxyphenyl)pyridin-2-amine

The titled compound was prepared in accordance with the same procedures as in Preparation C1-1, changing the amine reagent to 6-bromopyridin-2-amine. 1H NMR (CDCl₃, 400 MHz) δ 7.52 (t, 1H), 7.34-7.28 (m, 1H), 6.80-6.74 (m, 3H), 6.51 (d, 1H), 4.48 (brs, 2H), 3.79 (s, 3H)

### Preparation C1-9: 2-(2-methoxypyridin-3-yl)pyrimidin-4-amine

The titled compound was prepared in accordance with the same procedures as in Preparation C1-1, changing the boronic acid reagent to 2-methoxy-3-pyridylboronic acid reagent. 1H NMR (CDCl₃, 400 MHz) 8.39 (d, 1H), 8.25 (s, 1H), 8.01 (d, 1H), 6.99 (t, 1H), 6.40 (d, 1H), 4.97 (s, 2H), 4.03 (s, 3H)

### Preparation C1-10: methyl 4-(4-aminopyrimidin-2-yl)-3-fluoro-5-methoxybenzoate

The titled compound was prepared in accordance with the same procedures as in Preparation C1-1, changing the boronic acid reagent to (methyl 3-fluoro-5-methoxy-4-(4,4,5,5-tetramethyl-1,3,2-dioxaborolan-2-yl)benzoate. 1H NMR (CD₃OD, 400 MHz) 8.17 (d, 1H), 7.55 (s, 1H), 7.46 (d, 1H), 6.56 (d, 1H), 3.96 (s, 3H), 3.87 (s, 3H)

### Preparation C1-11: [2-(4-aminopyrimidin-2-yl)-3-fluoro-phenyl]methanol

The titled compound was prepared in accordance with the same procedures as in Preparation C1-1, changing the boronic acid reagent to [2-fluoro-6-(hydroxymethyl)phenyl]boronic acid. 1H NMR (CD₃OD, 400 MHz) δ 8.22 (d, 1H), 7.53-7.43 (m, 1H), 7.43-7.38 (m, 1H), 7.18-7.11 (m, 1H), 6.60 (d, 1H), 4.54 (s, 2H)

### Preparation C2-1: methyl 6-amino-2-(2-fluoro-6-methoxyphenyl)nicotinate

Methyl 6-amino-2-chloronicotinate (1.0 g, 5.36 mmol) was dissolved in anhydrous acetonitrile (30.0 mL) and then 2-fluoro-6-methoxyphenylboronic acid (1.82 g, 10.72 mmol), tetrakis(triphenylphosphine)palladium(0) (400.0 mg, 0.35 mmol), and potassium carbonate (2.22 g, 16.08 mmol) were added dropwise thereto. Then, the mixed solution was stirred at 100 °C for 2 hours. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (1.1 g). (Yield: 78.5%) 1H NMR (CDCl₃, 400 MHz) δ 8.15 (d, 1H), 7.34-7.32 (m, 1H), 6.81-6.75 (m, 2H), 6.51 (d, 1H), 4.94 (brs, 2H), 3.76 (s, 3H), 3.66 (s, 3H)

### Preparation C3-1: 6-(2-fluoro-6-methoxyphenyl)-5-nitropyridin-2-amine

6-Chloro-5-nitro-pyridin-2-amine (1.0 g, 5.76 mmol) was dissolved in anhydrous acetonitrile (30.0 mL) and then 2-fluoro-6-methoxyphenylboronic acid (1.82 g, 10.72 mmol), tetrakis(triphenylphosphine)palladium(0) (400.0 mg, 0.35 mmol), and potassium carbonate (2.22 g, 16.08 mmol) were added dropwise thereto. Then, the mixed solution was stirred at 100 °C for 2 hours. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (1.17 g). (Yield: 82.9%) 1H NMR (CDCl₃, 400 MHz) δ 8.30 (d, 1H), 7.39-7.35 (m, 1H), 6.83 (t, 1H), 6.77 (d, 1H), 6.54 (d, 1H), 5.22 (brs, 2H), 3.76 (s, 3H)

### Preparation C3-2: 6-(2,4-difluoro-6-methoxyphenyl)-5-nitropyridin-2-amine

The titled compound was prepared in accordance with the same procedures as in Preparation C3-1, changing the boronic acid reagent to (2,4-difluoro-6-methoxy-phenyl)boronic acid. 1H NMR (CD₃OD, 400 MHz) 8.26 (d, 1H), 6.75 (d, 1H), 6.66-6.61 (m, 2H), 3.74 (s, 3H)

### Preparation C4-1: 4-(4-aminopyrimidin-2-yl)-3-fluoro-5-methoxybenzaldehyde

2-Bromopyrimidin-4-amine (1.9 g, 11.11 mmol) was dissolved in anhydrous acetonitrile (44.9 mL) and water (12.5 mL) and then (2-fluoro-4-formyl-6-methoxy-phenyl)boronic acid (2.0 g, 10.1 mmol), XPhos Pd G2 (794.9 mg, 1.01 mmol), and potassium phosphate tribasic (6.4 g, 30.31 mmol) were added dropwise thereto. Then, the mixed solution was stirred at 100 °C for 4 hours. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (414.6 mg). (Yield: 16.6%) 1H NMR (CDCl₃, 400 MHz) δ 9.97 (s, 1H), 8.40 (d, 1H), 7.33-7.27 (m, 2H), 6.46 (d, 1H), 5.02 (brs, 2H), 3.88 (s, 3H)

### Preparation C4-2: 5-(4-aminopyrimidin-2-yl)thiophen-3-carbaldehyde

The titled compound was prepared in accordance with the same procedures as in Preparation C4-1, changing the boronic acid reagent to (4-formylthiophen-2-yl)boronic acid. 1H NMR (CD₃OD, 400 MHz) δ 9.86 (s, 1H), 8.45 (s, 1H), 8.19 (s, 1H), 8.08 (d, 1H), 6.41 (d, 1H)

### Preparation C5-1: (4-(4-aminopyrimidin-2-yl)-3-fluoro-5-methoxyphenyl)methanol

4-(4-Aminopyrimidin-2-yl)-3-fluoro-5-methoxybenzaldehyde (the C4-1 intermediate, 100.0 mg, 0.40 mmol) was dissolved in anhydrous tetrahydrofuran (6.0 mL) and then sodium borohydride (15.3 mg, 0.40 mmol) was added dropwise thereto. Then, the mixed solution was stirred at room temperature for 1 hour. After completion of the reaction, the reaction mixture was diluted with dichloromethane and then water was slowly added thereto. The extracted organic layer was dried over anhydrous magnesium sulfate and then filtered. The filtrate was concentrated to prepare the titled compound (69.0 mg), without further purification. (Yield: 68.4%) 1H NMR (CD₃OD, 400 MHz) δ 8.14 (d, 1H), 6.91 (s, 1H), 6.81 (d, 1H), 6.53 (d, 1H), 4.67 (s, 2H), 3.8 (s, 3H)

### Preparation C5-2: (5-(4-aminopyrimidin-2-yl)thiophen-3-yl)methanol

The titled compound was prepared in accordance with the same procedures as in Preparation C5-1, using the C4-2 intermediate. 1H NMR (CD₃OD, 400 MHz) 8.04 (d, 1H), 7.82 (s, 1H), 7.38 (s, 1H), 6.36 (d, 1H), 4.60 (s, 2H)

### Preparation C6-1: 2-(4-((dimethylamino)methyl)-2-fluoro-6-methoxyphenyl)pyrimidin-4-amine

4-(4-Aminopyrimidin-2-yl)-3-fluoro-5-methoxybenzaldehyde (the C4-1 intermediate, 100 mg, 0.4 mmol) was dissolved in methanol (2.0 mL) and then 2.0M dimethylamine dissolved in tetrahydrofuran (0.4 mL, 0.8 mmol) and sodium acetate (33.2 mg, 0.40 mmol) were added dropwise thereto. Then, the mixed solution was stirred at room temperature overnight. Sodium borohydride (15.3 mg, 0.40 mmol) was added at room temperature to the reaction mixture, which was then stirred for 1 hour. After completion of the reaction, dichloromethane was added to the reaction mixture and then a saturated sodium hydrogen carbonate solution was added thereto. The extracted organic layer was dried over anhydrous magnesium sulfate and then filtered. The filtrate was concentrated to prepare the titled compound (96.0 mg), without further purification. (Yield: 85.9%) 1H NMR (CD₃OD, 400 MHz) δ 8.14 (d, 1H), 6.92 (s, 1H), 6.81 (d, 1H), 6.53 (d, 1H), 3.8 (s, 3H), 3.54 (s, 2H), 2.30-2.28 (m, 6H)

### Preparation C6-2: 1-((5-(4-aminopyrimidin-2-yl)thiophen-3-yl)methyl)azetidin-3-ol

The titled compound was prepared in accordance with the same procedures as in Preparation C6-1, using the C4-2 intermediate and changing the amine reagent to 3-hydroxyazetidine hydrochloride. The product was used in the subsequent reaction without further purification.

### Preparation C7: 3-(6-amino-2-pyridyl)oxazolidin-2-one

6-Bromopyridin-2-amine (300.0 mg, 1.734 mmol) was dissolved in anhydrous 1,4-dioxane (3.0 mL) and then oxazolidin-2-one (151.0 mg, 1.734 mmol), palladium(II) acetate (38.9 mg, 0.173 mmol), XantPhos (150.5 mg, 0.26 mmol), and cesium carbonate (1.41 g, 4.335 mmol) were added dropwise thereto. Then, the mixed solution was stirred at 100 °C overnight. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (63.4 mg). (Yield: 20.4%) 1H NMR (CDCl₃, 400 MHz) δ 7.55 (d, 1H), 7.47 (t, 1H), 6.25 (d, 1H), 4.45 (t, 2H), 4.40-4.30 (s, 2H), 4.22 (t, 2H)

### Preparation C8-1: 6-amino-2-(2-fluoro-6-methoxy-phenyl)pyridin-3-carbonitrile

6-Amino-2-chloro-pyridin-3-carbonitrile (500.0 mg, 3.26 mmol) was dissolved in anhydrous 1,4-dioxane (5.0 mL) and water (1.0 mL) and then 2-fluoro-6-methoxyphen ylboronic acid (608.7 mg, 3.58 mmol), tetrakis(triphenylphosphine)palladium(0) (376.2 mg, 0.33 mmol), and potassium carbonate (899.9 mg, 6.51 mmol) were added dropwise thereto. Then, the mixed solution was stirred at 100 °C for 17 hours. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (355.8 mg, Yield: 44.9%). 1H NMR (CDCl₃, 400 MHz) δ 7.72 (d, 1H), 7.39 (q, 1H), 6.86-6.80 (m, 2H), 6.51 (d, 1H), 5.11 (s, 2H), 3.85 (s, 3H)

### Preparation C8-2: 6-amino-2-(2,4-difluoro-6-methoxy-phenyl)pyridin-3-carbonitrile

The titled compound was prepared in accordance with the same procedures as in Preparation C8-1, changing the boronic acid reagent to (2,4-difluoro-6-methoxyphenyl)boronic acid. 1H NMR (CDCl₃, 400 MHz) 7.73 (d, 1H), 6.62-6.51 (m, 3H), 5.04 (s, 2H), 3.84 (s, 3H)

### Example 1: (S)-cyclopropyl(4-(3-hydroxypiperidin-1-yl)-6-((2-(5-methylpyridin-3-yl)pyrimidin-4-yl)amino)pyridin-3-yl)methanone

(S)-(6-((2-Chloropyrimidin-4-yl)amino)-4-(3-hydroxypiperidin-1-yl)pyridin-3-yl) (cyclopropyl)methanone (Intermediate A1-1, 15.0 mg, 0.04 mmol) was dissolved in anhydrous 1,4-dioxane (0.4 mL) and then (5-methylpyridin-3-yl)boronic acid (10.9 mg, 0.08 mmol), bis(triphenylphosphine)palladium(II) dichloride (1.4 mg, 0.002 mmol), and 2.0M sodium carbonate solution (0.1 mL, 0.20 mmol) were added dropwise thereto. Then, the mixed solution was stirred in a microwave reactor for 1 hour. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (3.7 mg). (Yield: 21.4%) 1H NMR (CDCl₃, 400 MHz) δ 9.59 (s, 1H), 8.52-8.49 (m, 3H), 8.34-8.32 (m, 2H), 7.94 (s, 1H), 6.78 (d, 1H), 5.82-5.52 (brs, 1H), 4.13-4.05 (m, 2H), 3.31-3.28 (m, 1H), 3.14-3.11 (m, 2H), 2.54-2.51 (m, 1H), 2.46 (s, 3H), 2.18-2.15 (m, 1H), 1.80-1.79 (m, 1H), 1.65-1.63 (m, 2H), 1.28-1.25 (m, 2H), 1.09-1.07 (m, 1H), 1.06-1.01 (m, 1H) MS m/z=431.1 (M+H)+

### Example 2: (S)-(6-((2-(5-aminopyridin-3-yl)pyrimidin-4-yl)amino)-4-(3-hydroxypiperidin-1-yl)pyridin-3-yl)(cyclopropyl)methanone

The titled compound (6.8 mg) was prepared in accordance with the same procedures as in Example 1, changing the boronic acid reagent to 3-aminopyridin-5-boronic acid pinacol ester. (Yield: 38.9%) 1H NMR (CDCl₃, 400 MHz) δ 9.17 (s, 1H), 8.50-8.49 (d, 1H), 8.36 (s, 1H), 8.3 (s, 1H), 8.13 (s, 1H), 8 (s, 1H), 7.89 (brs, 1H), 6.74 (d, 1H), 4.12-4.10 (m, 1H), 4.07 (brs, 2H), 3.32-3.27 (m, 1H), 3.12-3.06 (m, 2H), 2.54-2.52 (m, 1H), 2.15-2.12 (m, 1H), 1.81-1.79 (m, 1H), 1.54-1.51 (m, 2H), 1.28-1.26 (m, 2H), 1.26-1.25 (m, 1H), 1.07-1.06 (m, 1H) MS m/z=432.0 (M+H)+

### Example 3: (S)-cyclopropyl(4-(3-hydroxypiperidin-1-yl)-6-((2-(5-methoxypyridin-3-yl)pyrimidin-4-yl)amino)pyridin-3-yl)methanone

The titled compound (4.4 mg) was prepared in accordance with the same procedures as in Example 1, changing the boronic acid reagent to 3-methoxy-5-(4,4,5,5-tetramethyl-1,3,2-dioxaborolan-2-yl)pyridine. (Yield: 24.5%) 1H NMR (CDCl₃, 400 MHz) δ 9.38 (s, 1H), 8.52 (d, 1H), 8.37 (s, 1H), 8.36 (s, 1H), 8.32 (s, 1H), 8.29 (s, 1H), 8.23 (s, 1H), 8.22 (s, 1H), 6.8 (d, 1H), 5.41-5.31 (brs, 1H), 4.13-4.10 (m, 2H), 4.05 (s, 3H), 3.31-3.28 (m, 1H), 3.13-3.10 (m, 2H), 2.55-2.52 (m, 1H), 2.18-2.15 (m, 1H), 1.81-1.80 (m, 1H), 1.60-1.56 (m, 2H), 1.28-1.25 (m, 2H), 1.09-1.07 (m, 1H), 1.06-1.02 (m, 1H) MS m/z=447.0 (M+H)+

### Example 4: (S)-cyclopropyl(6-((2-(3-hydroxyphenyl)pyrimidin-4-yl)amino)-4-(3-hydroxypiperidin-1-yl)pyridin-3-yl)methanone

The titled compound (1.4 mg) was prepared in accordance with the same procedures as in Example 1, changing the boronic acid reagent to 3-hydroxyphenylboronic acid. (Yield: 8.0%) 1H NMR (CD₃OD, 400 MHz) δ 8.46 (d, 1H), 8.24 (s, 1H), 7.99 (s, 1H), 7.85-7.81 (m, 2H), 7.35-7.28 (m, 2H), 6.96-6.93 (m, 1H), 3.90-3.88 (m, 1H), 3.57-3.54 (m, 1H), 3.46-3.43 (m, 1H), 3.02-2.91 (m, 1H), 2.89-2.83 (m, 2H), 2.03-1.96 (m, 1H), 1.82-1.62 (m, 1H), 1.50-1.45 (m, 1H), 1.29-1.20 (m, 2H), 1.19-1.12 (m, 2H) MS m/z=432.1 (M+H)+

### Example 5: (S)-cyclopropyl(6-((2-(2-fluoro-6-methoxyphenyl)pyrimidin-4-yl)amino)-4-(3-hydroxypiperidin-1-yl)pyridin-3-yl)methanone

The titled compound (1.4 mg) was prepared in accordance with the same procedures as in Example 1, changing the boronic acid reagent to 2-fluoro-6-methoxyphenylboronic acid. (Yield: 7.4%) 1H NMR (CDCl₃, 400 MHz) δ 8.62 (d, 1H), 8.40 (s, 1H), 7.82 (s, 1H), 7.54 (s, 1H), 7.39-7.30 (m, 2H), 6.84-6.80 (m, 2H), 3.88-3.86 (m, 1H), 3.82 (s, 3H), 3.34-3.30 (m, 1H), 3.13-3.07 (m, 1H), 3.04-3.02 (m, 1H), 3.01-2.99 (m, 2H), 2.62-2.58 (m, 1H), 2.24-2.21 (m, 1H), 1.90-1.84 (m, 2H), 1.31-1.26 (m, 2H), 1.04-1.02 (m, 2H) MS m/z=464.1 (M+H)+

### Example 6: cyclopropyl(6-((2-(2-fluoro-6-methoxyphenyl)pyrimidin-4-yl)amino)-4-(3-hydroxyazetidin-1-yl)pyridin-3-yl)methanone

### Step 1: (6-chloro-4-(3-hydroxyazetidin-1-yl)pyridin-3-yl)(cyclopropyl)methanone

Cyclopropyl(4,6-dichloropyridin-3-yl)methanone (1.0 g, 4.62 mmol) was dissolved in anhydrous dimethylformamide (23.1 mL) and then 3-hydroxyazetidine hydrochloride (0.61 g, 5.55 mmol) and diisopropylethylamine (1.26 mL, 9.25 mmol) were added dropwise thereto. The reaction mixture was stirred at room temperature overnight. Water was added to the reaction mixture and then the aqueous layer was extracted with ethyl acetate. The extracted organic layer was dried over anhydrous magnesium sulfate and then filtered. The filtrate was concentrated and then purified by silica gel column chromatography to prepare the titled compound (0.94 g). (Yield: 80.5%) 1H NMR (CDCl₃, 400 MHz) δ 8.46 (s, 1H), 6.31 (s, 1H), 4.75-4.70 (m, 1H), 4.23-4.19 (m, 2H), 3.81-3.77 (m, 2H), 3.03-3.02 (m, 1H), 2.44-2.38 (m, 1H), 1.24-1.21 (m, 2H), 1.11-1.08 (m, 2H)

### Step 2: cyclopropyl(6-((2-(2-fluoro-6-methoxyphenyl)pyrimidin-4-yl)amino)-4-(3-hydroxyazetidin-1-yl)pyridin-3-yl)methanone

(6-Chloro-4-(3-hydroxyazetidin-1-yl)pyridin-3-yl)(cyclopropyl)methanone (63.4 mg, 0.25 mmol) was dissolved in anhydrous toluene (2.0 mL) and then 2-(2-fluoro-6-methoxyphenyl)pyrimidin-4-amine (Intermediate C1-1, 50.0 mg, 0.23 mmol), tris(dibenzylideneacetone)dipalladium(0) (20.8 mg, 0.02 mmol), 2-dicyclohexylphosphino-2′,4′,6′-triisopropylbiphenyl (21.7 mg, 0.04 mmol), and cesium carbonate (111.4 mg, 0.34 mmol) were added dropwise thereto. Then, the mixed solution was refluxed under stirring at 110 °C overnight. After completion of the reaction, the reaction mixture was cooled to room temperature and then water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (10 mg). (Yield: 8.8%) 1H NMR (CD₃OD, 400 MHz) δ 8.52 (s, 1H), 8.45 (d, 1H), 7.45-7.41 (m, 2H), 7.25 (s, 1H), 6.97 (dd, 1H), 6.87 (dd, 1H), 4.57-4.54 (m, 1H), 4.08-4.04 (m, 2H), 3.81 (s, 3H), 3.66-3.62 (m, 2H), 2.58-2.55 (m, 1H), 1.13-1.05 (m, 4H) MS m/z=436.0 (M+H)+

### Example 7: (S)-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-ol

(S)-1-(2-Chloro-5-(1-(tetrahydro-2H-pyran-4-yl)-1H-pyrazol-4-yl)pyridin-4-yl)piperidin-3-ol (Intermediate A2-1, 40.0 mg, 0.12 mmol) was dissolved in anhydrous 1,4-dioxane (0.5 mL) and then 2-(2-fluoro-6-methoxyphenyl)pyrimidin-4-amine (Intermediate C1-1, 20.9 mg, 0.09 mmol), tris(dibenzylideneacetone)dipalladium(0) (10.9 mg, 0.012 mmol), 2-dicyclohexylphosphino-2′,4′,6′-triisopropylbiphenyl (11.39 mg, 0.024 mmol), and cesium carbonate (77.8 mg, 0.24 mmol) were added dropwise thereto. Then, the mixed solution was stirred at 100 °C overnight. After completion of the reaction, the reaction mixture was cooled to room temperature and water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (10.6 mg). (Yield: 16.3%) 1H NMR (CDCl₃, 400 MHz) δ 8.4 (d, 1H), 8.09-8.08 (m, 2H), 7.83 (s, 1H), 7.60 (s, 1H), 7.51-7.50 (m, 1H), 7.44-7.42 (m, 1H), 6.96 (d, 1H), 6.84 (dd, 1H), 4.49-4.46 (m, 1H), 4.10-4.07 (m, 2H), 3.81 (s, 3H), 3.70-3.61 (m, 1H), 3.59-3.56 (m, 2H), 3.31-3.30 (m, 1H), 3.29-3.27 (m, 1H), 2.72-2.60 (m, 1H), 2.45-2.40 (m, 1H), 2.14-2.08 (m, 4H), 1.98-1.97 (m, 1H), 1.33-1.31 (m, 1H), 1.30-1.29 (m, 1H) MS m/z=546.2 (M+H)+

### Example 8: (S)-1-(2-((2-(2-fluoro-6-methoxyphenyl)pyrimidin-4-yl)amino)-5-((1-(trifluoromethyl)-1H-pyrazol-4-yl)ethynyl)pyridin-4-yl)piperidin-3-ol

The titled compound (2.6 mg) was prepared in accordance with the same procedures as in Example 7, using Intermediates A6-1 and C1-1. (Yield: 7.9%) 1H NMR (CDCl₃, 400 MHz) δ 8.60 (d, 1H), 8.23 (s, 1H), 8.06 (s, 1H), 7.90 (s, 1H), 7.78 (s, 1H), 7.45 (s, 1H), 7.37-7.31 (m, 2H), 6.83-6.80 (m, 2H), 3.96-3.95 (m, 1H), 3.82 (s, 3H), 3.45-3.44 (m, 2H), 3.30-3.24 (m, 2H), 1.92-1.83 (m, 4H) MS m/z=554.1 (M+H)+

### Example 9: (R)-1-(2-((2-(2-fluoro-6-methoxyphenyl)pyrimidin-4-yl)amino)-5-((1-(trifluoromethyl)-1H-pyrazol-4-yl)ethynyl)pyridin-4-yl)piperidin-3-ol

The titled compound (2.8 mg) was prepared in accordance with the same procedures as in Example 7, using Intermediates B5-2 and C1-1. (Yield: 7.9%) 1H NMR (CDCl₃, 400 MHz) δ 8.60 (d, 1H), 8.22 (s, 1H), 8.06 (s, 1H), 7.97 (s, 1H), 7.90 (s, 1H), 7.49 (s, 1H), 7.37-7.31 (m, 1H), 7.29-7.27 (m, 1H), 6.83-6.80 (m, 2H), 3.95-9.94 (m, 1H), 3.84 (s, 3H), 3.45-3.42 (m, 2H), 3.33-3.28 (m, 1H), 3.24-3.21 (m, 1H), 1.92-1.83 (m, 4H) MS m/z=554.1 (M+H)+

### Example 10: 1-(2-((2-(2-fluoro-6-methoxyphenyl)pyrimidin-4-yl)amino)-5-((tetrahydro-2H-pyran-4-yl)ethynyl)pyridin-4-yl)azetidin-3-ol

The titled compound (7.5 mg) was prepared in accordance with the same procedures as in Example 7, using Intermediate B5-1 and C1-1. (Yield: 15.4%) 1H NMR (CD₃OD, 400 MHz) δ 8.39 (d, 1H), 7.89 (s, 1H), 7.44-7.40 (m, 1H), 7.37 (d, 1H), 6.99 (s, 1H), 6.96 (d, 1H), 6.85 (dd, 1H), 4.59-4.57 (m, 1H), 4.41-4.39 (m, 2H), 3.93-3.88 (m, 4H), 3.81 (s, 3H), 3.56-3.50 (m, 2H), 2.97-2.87 (m, 1H), 1.92-1.87 (m, 2H), 1.74-1.68 (m, 2H) MS m/z=476.1 (M+H)+

### Example 11: (S)-1-(2-((2-(2-fluoro-6-methoxyphenyl)pyrimidin-4-yl)amino)-5-((tetrahydro-2H-pyran-4-yl)ethynyl)pyridin-4-yl)piperidin-3-ol

The titled compound (6.0 mg) was prepared in accordance with the same procedures as in Example 7, using Intermediates A6-2 and C1-1. (Yield: 11.6%) 1H NMR (CD₃OD, 400 MHz) δ 8.42 (d, 1H), 8.06 (s, 1H), 7.51 (s, 1H), 7.46-7.42 (m, 2H), 6.96 (d, 1H), 6.85 (dd, 1H), 3.95-3.94 (m, 1H), 3.93-3.90 (m, 2H), 3.81 (s, 3H), 3.58-3.55 (m, 1H), 3.54-3.52 (m, 3H), 3.01-2.98 (m, 1H), 2.98-2.94 (m, 1H), 2.69-2.63 (m, 1H), 1.96-1.95 (m, 1H), 1.93-1.91 (m, 2H), 1.79-1.73 (m, 3H), 1.62-1.58 (m, 1H), 1.33-1.29 (m, 1H) MS m/z=504.2 (M+H)+

### Example 12: (S)-1-(2-((2-(2-fluoro-6-methoxyphenyl)pyrimidin-4-yl)amino)-5-(1-methyl-1H-pyrazol-4-yl)pyridin-4-yl)piperidin-3-ol

The titled compound (17.4 mg) was prepared in accordance with the same procedures as in Example 7, using Intermediates A2-2 and C1-1. (Yield: 32.3%) 1H NMR (CD₃OD, 400 MHz) δ 8.39 (d, 1H), 8.07 (s, 1H), 7.93 (s, 1H), 7.8 (s, 1H), 7.58 (s, 1H), 7.51-7.50 (m, 1H), 7.46-7.40 (m, 1H), 6.93 (d, 1H), 6.81 (dd, 1H), 3.81 (s, 3H), 3.7 (s, 3H), 3.69-3.68 (m, 1H), 3.32-3.31 (m, 1H), 3.28-3.27 (m, 1H), 3.06-3.03 (m, 1H), 2.62-2.58 (m, 1H), 2.42-2.37 (m, 1H), 1.97-1.96 (m, 1H), 1.70-1.69 (m, 1H), 1.59-1.58 (m, 1H), 1.34-1.32 (m, 1H) MS m/z=476.1 (M+H)+

### Example 13: (S)-1-(2-((2-(2-fluoro-6-methoxyphenyl)pyrimidin-4-yl)amino)-5-(1-isopropyl-1H-pyrazol-4-yl)pyridin-4-yl)piperidin-3-ol

The titled compound (2.0 mg) was prepared in accordance with the same procedures as in Example 7, using Intermediates A2-3 and C1-1. (Yield: 2.9%) 1H NMR (CD₃OD, 400 MHz) δ 8.41 (d, 1H), 8.1 (s, 1H), 8.05 (s, 1H), 7.81 (s, 1H), 7.61 (s, 1H), 7.51-7.50 (m, 1H), 7.44-7.42 (m, 1H), 6.96 (d, 1H), 6.86 (dd, 1H), 4.60-4.56 (m, 1H), 3.82 (s, 3H), 3.79-3.69 (m, 1H), 3.29-3.28 (m, 1H), 3.06-3.03 (m, 1H), 2.63-2.60 (m, 1H), 2.45-2.40 (m, 1H), 1.96-1.93 (m, 1H), 1.55-1.53 (m, 1H), 1.36-1.33 (m, 1H), 1.29 (d, 6H), 0.92-0.88 (m, 2H) MS m/z=504.2 (M+H)+

### Example 14: (3S)-1-(2-((2-(2-fluoro-6-methoxyphenyl)pyrimidin-4-yl)amino)-5-(1-(tetrahydrofuran-3-yl)-1H-pyrazol-4-yl)pyridin-4-yl)piperidin-3-ol

The titled compound (9.3 mg) was prepared in accordance with the same procedures as in Example 7, using Intermediates A2-4 and C1-1. (Yield: 12.8%) 1H NMR (CD₃OD, 400 MHz) δ 8.41 (d, 1H), 8.09 (s, 1H), 8.06 (s, 1H), 7.83 (s, 1H), 7.6 (s, 1H), 7.51-7.50 (m, 1H), 7.44-7.42 (m, 1H), 6.96 (d, 1H), 6.86 (dd, 1H), 5.08-5.07 (m, 1H), 4.15-4.11 (m, 1H), 4.09-4.04 (m, 2H), 3.95-3.91 (m, 1H), 3.81 (s, 3H), 3.70-3.68 (m, 1H), 3.31-3.27 (m, 1H), 3.06-3.03 (m, 1H), 2.59-2.49 (m, 2H), 2.40-2.35 (m, 2H), 1.97-1.93 (m, 1H), 1.88-1.71 (m, 1H), 1.59-1.57 (m, 1H), 1.34-1.29 (m, 1H) MS m/z=532.2 (M+H)+

### Example 15: (S)-1-(2-((2-(2-fluoro-6-methoxyphenyl)pyrimidin-4-yl)amino)-5-(1-(1-methylpiperidin-4-yl)-1H-pyrazol-4-yl)pyridin-4-yl)piperidin-3-ol

The titled compound (6.2 mg) was prepared in accordance with the same procedures as in Example 7, using Intermediates A2-5 and C1-1. (Yield: 8.1%) 1H NMR (CD₃OD, 400 MHz) δ 8.41 (d, 1H), 8.1 (d, 2H), 7.82 (s, 1H), 7.61 (s, 1H), 7.51-7.50 (m, 1H), 7.44-7.42 (m, 1H), 6.96 (d, 1H), 6.86 (dd, 1H), 4.25-4.22 (m, 1H), 3.82 (s, 3H), 3.72-3.70 (m, 1H), 3.04-3.02 (m, 3H), 2.62-2.59 (m, 1H), 2.57-2.41 (m, 1H), 2.39 (s, 3H), 2.35-2.27 (m, 2H), 2.24-2.12 (m, 4H), 1.95-1.93 (m, 1H), 1.74-1.71 (m, 1H), 1.58-1.55 (m, 1H), 1.35-1.33 (m, 1H) MS m/z=559.3 (M+H)+

### Example 16: (S)-1-(2-((2-(2-fluoro-6-methoxyphenyl)pyrimidin-4-yl)amino)-5-(1-(2,2,2-trifluoroethyl)-1H-pyrazol-4-yl)pyridin-4-yl)piperidin-3-ol

The titled compound (6.4 mg) was prepared in accordance with the same procedures as in Example 7, using Intermediates A2-6 and C1-1. (Yield: 8.6%) 1H NMR (CD₃OD, 400 MHz) δ 8.42 (d, 1H), 8.13 (d, 2H), 7.94 (s, 1H), 7.63 (s, 1H), 7.53-7.52 (m, 1H), 7.44-7.43 (m, 1H), 6.96 (d, 1H), 6.86 (dd, 1H), 5.02 (q, 2H), 3.82 (s, 3H), 3.79-3.70 (m, 1H), 3.31-3.26 (m, 1H), 3.05-3.02 (m, 1H), 2.62-2.57 (m, 1H), 2.48-2.43 (m, 1H), 1.95-1.93 (m, 1H), 1.72-1.60 (m, 1H), 1.57-1.54 (m, 1H), 1.35-1.29 (m, 1H) MS m/z=544.2 (M+H)+

### Example 17: (S)-1-(5-(1-(cyclopropylmethyl)-1H-pyrazol-4-yl)-2-((2-(2-fluoro-6-methoxyphenyl)pyrimidin-4-yl)amino)pyridin-4-yl)piperidin-3-ol

The titled compound (2.8 mg) was prepared in accordance with the same procedures as in Example 7, using Intermediates A2-7 and C1-1. (Yield: 3.9%) 1H NMR (CD₃OD, 400 MHz) δ 8.42 (d, 1H), 8.10 (d, 2H), 7.81 (s, 1H), 7.61 (s, 1H), 7.51-7.50 (m, 1H), 7.44-7.43 (m, 1H), 6.96 (d, 1H), 6.86 (dd, 1H), 4.06 (d, 2H), 3.84 (s, 3H), 3.74-3.70 (m, 1H), 3.07-3.04 (m, 1H), 2.63-2.58 (m, 1H), 2.45-2.40 (m, 1H), 1.97-1.93 (m, 1H), 1.75-1.71 (m, 1H), 1.62-1.56 (m, 2H), 1.35-1.33 (m, 1H), 0.95-0.92 (m, 1H), 0.62-0.51 (m, 2H), 0.42-0.32 (m, 2H) MS m/z=516.2 (M+H)+

### Example 18: (S)-1-(2-((2-(2-fluoro-6-methoxyphenyl)pyrimidin-4-yl)amino)-5-(4-(tetrahydro-2H-pyran-4-yl)phenyl)pyridin-4-yl)piperidin-3-ol

The titled compound (14.4 mg) was prepared in accordance with the same procedures as in Example 7, using Intermediates A2-12 and C1-1. (Yield: 18.9%) 1H NMR (CD₃OD, 400 MHz) δ 8.42 (d, 1H), 7.90 (s, 1H), 7.56 (s, 1H), 7.52-7.40 (m, 5H), 7.33-7.31 (m, 2H), 6.96 (d, 1H), 6.86 (dd, 1H), 4.07-4.04 (m, 2H), 3.82 (s, 3H), 3.79-3.31 (m, 3H), 3.27-3.26 (m, 1H), 3.09-3.06 (m, 1H), 2.84-2.81 (m, 1H), 2.56-2.53 (m, 1H), 2.40-2.35 (m, 1H), 1.90-1.79 (m, 5H), 1.57-1.53 (m, 1H), 1.42-1.24 (m, 4H) MS m/z=556.2 (M+H)+

### Example 19: (S)-1-(2-((2-(2-fluoro-6-methoxyphenyl)pyrimidin-4-yl)amino)-5-(4-(4-methylpiperazin-1-yl)phenyl)pyridin-4-yl)piperidin-3-ol

The titled compound (8.1 mg) was prepared in accordance with the same procedures as in Example 7, using Intermediates A2-13 and C1-1. (Yield: 10.4%) 1H NMR (CD₃OD, 400 MHz) δ 8.41 (d, 1H), 7.88 (s, 1H), 7.52-7.50 (m, 2H), 7.43-7.40 (m, 3H), 7.03 (d, 2H), 6.95 (d, 1H), 6.86 (dd, 1H), 3.81 (s, 3H), 3.56-3.54 (m, 1H), 3.31-3.24 (m, 5H), 3.10-3.07 (m, 1H), 2.65-2.64 (m, 4H), 2.55-2.51 (m, 1H), 2.37 (s, 3H), 2.34-2.32 (m, 1H), 1.92-1.55 (m, 1H), 1.62-1.55 (m, 1H), 1.29-1.26 (m, 1H) MS m/z=570.3 (M+H)+

### Example 20: (S)-1-(6-((2-(2-fluoro-6-methoxyphenyl)pyrimidin-4-yl)amino)-6′-morpholino-[3,3′-bipyridin]-4-yl)piperidin-3-ol

The titled compound (8.6 mg) was prepared in accordance with the same procedures as in Example 7, using Intermediates A2-14 and C1-1. (Yield: 11.3%) 1H NMR (CD₃OD, 400 MHz) δ 8.42 (d, 1H), 8.29 (s, 1H), 7.92 (s, 1H), 7.83-7.81 (m, 1H), 7.59 (s, 1H), 7.54-7.52 (m, 1H), 7.46-7.40 (m, 1H), 6.96 (d, 1H), 6.91 (d, 1H), 6.86 (dd, 1H), 3.82-8.80 (m, 7H), 3.56-3.55 (m, 1H), 3.54-3.50 (m, 4H), 3.31-3.28 (m, 1H), 3.10-3.07 (m, 1H), 2.59-2.53 (m, 1H), 2.40-2.35 (m, 1H), 1.93-1.90 (m, 1H), 1.64-1.61 (m, 1H), 1.48-1.45 (m, 1H), 1.33-1.29 (m, 1H) MS m/z=558.2 (M+H)+

### Example 21: (S)-1-(2-((2-(4-(hydroxymethyl)thiophen-2-yl)pyrimidin-4-yl)amino)-5-(1-(tetrahydro-2H-pyran-4-yl)-1H-pyrazol-4-yl)pyridin-4-yl)piperidin-3-ol

The titled compound (6.7 mg) was prepared in accordance with the same procedures as in Example 7, using Intermediates A2-1 and C5-2. (Yield: 15.2%) 1H NMR (CDCl₃, 400 MHz) δ 8.41-8.40 (m, 1H), 8.29-8.06 (m, 3H), 7.85 (d, 2H), 7.48-7.47 (m, 1H), 7.31 (s, 1H), 6.62-6.61 (m, 1H), 4.71 (s, 2H), 4.45-4.41 (m, 1H), 4.17-4.15 (m, 2H), 3.62-3.61 (m, 1H), 3.59-3.57 (m, 3H), 3.18-3.15 (m, 1H), 2.89-2.86 (m, 1H), 2.47-2.44 (m, 1H), 2.20-2.05 (m, 6H), 1.71-1.67 (m, 1H), 1.49-1.43 (m, 1H) MS m/z=534.2 (M+H)+

### Example 22: (S)-1-(2-((2-(2,6-difluorophenyl)pyrimidin-4-yl)amino)-5-(1-(tetrahydro-2H-pyran-4-yl)-1H-pyrazol-4-yl)pyridin-4-yl)piperidin-3-ol

(3S)-1-[2-[(2-Chloropyrimidin-4-yl)amino]-5-(1-tetrahydropyran-4-ylpyrazol-4-yl)-4-pyridyl]piperidin-3-ol (Intermediate A1-2, 20.0 mg, 0.04 mmol) was dissolved in anhydrous 1,4-dioxane (0.6 mL) and water (0.2 mL) and then 2-(2,6-difluorophenyl)-4,4,5,5-tetramethyl-1,3,2-dioxaborolane (21.0 mg, 0.08 mmol), XPhos Pd G2 (3.45 mg, 0.004 mmol), and K₃PO₄(27.9 mL, 0.130 mmol) were added thereto. Then, the mixed solution was stirred in a microwave reactor for 1 hour. After completion of the reaction, the reaction mixture was cooled to room temperature and water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (3.1 mg). (Yield: 13.2%) 1H NMR (CD₃OD, 400 MHz) δ 8.58 (s, 1H), 8.25 (s, 1H), 7.95 (d, 1H), 7.88 (s, 1H), 7.35-7.33 (m, 1H), 7.12 (s, 2H), 7.03-6.98 (m, 1H), 6.04 (d, 1H), 5.09-5.07 (m, 1H), 5.03 (s, 1H), 4.47-4.37 (m, 2H), 4.19-4.12 (m, 3H), 3.71-3.55 (m, 1H), 3.07-3.05 (m, 2H), 2.96-2.91 (m, 1H), 2.52-2.40 (m, 3H), 2.16-2.03 (m, 1H), 1.92-1.91 (m, 1H), 1.81-1.78 (m, 1H), 1.64-1.58 (m, 1H) MS m/z=534.2 (M+H)+

### Example 23: (S)-1-(2-((2-(3,6-difluoro-2-methoxyphenyl)pyrimidin-4-yl)amino)-5-(1-(tetrahydro-2H-pyran-4-yl)-1H-pyrazol-4-yl)pyridin-4-yl)piperidin-3-ol

The titled compound (8.2 mg) was prepared in accordance with the same procedures as in Example 22, changing the boronic acid reagent to 2-(3,6-difluoro-2-methoxy-phenyl)-4,4,5,5-tetramethyl-1,3,2-dioxaborolane. (Yield: 33.1%) 1H NMR (CD₃OD, 400 MHz) δ 8.56 (s, 1H), 8.26 (s, 1H), 7.95 (d, 1H), 7.89 (s, 1H), 7.08-7.06 (m, 2H), 6.87-6.84 (m, 1H), 6.04 (d, 1H), 5.09-5.07 (m, 1H), 4.57-4.47 (m, 1H), 4.19-4.12 (m, 2H), 3.89 (s, 3H), 3.61-3.55 (m, 3H), 3.04-2.95 (m, 1H), 2.93-2.88 (m, 2H), 2.62-2.51 (m, 1H), 2.17-2.05 (m, 5H), 1.90-1.81 (m, 1H), 1.79-1.63 (m, 1H) MS m/z=564.2 (M+H)+

### Example 24: (S)-1-(2-((2-(2,3-difluoro-6-methoxyphenyl)pyrimidin-4-yl)amino)-5-(1-(tetrahydro-2H-pyran-4-yl)-1H-pyrazol-4-yl)pyridin-4-yl)piperidin-3-ol

The titled compound (4.7 mg) was prepared in accordance with the same procedures as in Example 22, changing the boronic acid reagent to (2,3-difluoro-6-methoxy-phenyl)boronic acid. (Yield: 19.0%) 1H NMR (CD₃OD, 400 MHz) δ 8.56 (s, 1H), 8.24 (s, 1H), 7.95 (d, 1H), 7.88 (s, 1H), 7.15-7.13 (m, 1H), 7.05 (s, 1H), 6.69-6.66 (m, 1H), 6.04 (d, 1H), 5.08-5.05 (m, 1H), 5.02 (s, 1H), 4.47-4.45 (m, 1H), 4.19-4.12 (m, 2H), 3.8 (s, 3H), 3.78-3.55 (m, 3H), 3.15-3.04 (m, 1H), 2.94-2.87 (m, 2H), 2.42-2.38 (m, 1H), 2.16-2.05 (m, 5H), 1.91-1.90 (m, 1H), 1.80-1.77 (m, 1H) MS m/z=564.2 (M+H)+

### Example 25: (S)-1-(2-((2-(2-ethoxy-6-fluorophenyl)pyrimidin-4-yl)amino)-5-(1-(tetrahydro-2H-pyran-4-yl)-1H-pyrazol-4-yl)pyridin-4-yl)piperidin-3-ol

The titled compound (4.9 mg) was prepared in accordance with the same procedures as in Example 22, changing the boronic acid reagent to 2-(2-ethoxy-6-fluoro-phenyl)-4,4,5,5-tetramethyl-1,3,2-dioxaborolane. (Yield: 19.9%) 1H NMR (CD₃OD, 400 MHz) δ 8.56 (s, 1H), 8.26 (s, 1H), 7.95 (d, 1H), 7.89 (s, 1H), 7.12 (s, 1H), 6.81-6.75 (m, 2H), 6.03 (d, 1H), 5.08-5.06 (m, 1H), 5.01 (s, 1H), 4.47-4.45 (m, 1H), 4.19-4.12 (m, 2H), 4.05 (q, 2H), 3.71-3.56 (m, 3H), 3.06-3.03 (m, 1H), 2.88-2.84 (m, 2H), 2.25-2.47 (m, 1H), 2.17-2.05 (m, 5H), 1.89-1.77 (m, 1H), 1.64-1.63 (m, 1H), 1.31 (t, 3H) MS m/z=560.3 (M+H)+

### Example 26: (S)-1-(2-((2-([1,2,4]triazolo[1,5-a]pyridin-7-yl)pyrimidin-4-yl)amino)-5-(1-(tetrahydro-2H-pyran-4-yl)-1H-pyrazol-4-yl)pyridin-4-yl)piperidin-3-ol

The titled compound (11.6 mg) was prepared in accordance with the same procedures as in Example 22, changing the boronic acid reagent to 7-(4,4,5,5-tetramethyl-1,3,2-dioxaborolan-2-yl)-[1,2,4]triazolo[1,5-a]pyridine. (Yield: 49.1%) 1H NMR (CD₃OD, 400 MHz) δ 8.66 (d, 1H), 8.64 (s, 1H), 8.57-8.41 (m, 2H), 8.21 (s, 1H), 8.04 (d, 1H), 7.94-7.85 (m, 2H), 7.63 (s, 1H), 6.10-6.09 (d, 1H), 5.18 (brs, 2H), 4.49-4.46 (m, 1H), 4.18-4.13 (m, 2H), 3.69-3.56 (m, 3H), 3.15-3.08 (m, 2H), 3.00-2.98 (m, 1H), 2.40-2.37 (m, 1H), 2.17-2.05 (m, 5H), 1.94-1.89 (m, 1H), 1.63-1.62 (m, 1H) MS m/z=539.2 (M+H)+

### Example 27: (S)-1-(2-((2-(pyrazolo[1,5-a]pyridin-3-yl)pyrimidin-4-yl)amino)-5-(1-(tetrahydro-2H-pyran-4-yl)-1H-pyrazol-4-yl)pyridin-4-yl)piperidin-3-ol

The titled compound (4.1 mg) was prepared in accordance with the same procedures as in Example 22, changing the boronic acid reagent to 3-(4,4,5,5-tetramethyl-1,3-dioxolan-2-yl)pyrazolo[1,5-a]pyridine. (Yield: 17.4%) 1H NMR (CD₃OD, 400 MHz) δ 8.66 (d, 1H), 8.64 (s, 1H), 8.57-8.41 (m, 2H), 8.21 (s, 1H), 8.04 (d, 1H), 7.94-7.85 (m, 2H), 7.63 (s, 1H), 6.10-6.09 (d, 1H), 5.18 (brs, 2H), 4.49-4.46 (m, 1H), 4.18-4.13 (m, 2H), 3.69-3.56 (m, 3H), 3.15-3.08 (m, 2H), 3.00-2.98 (m, 1H), 2.40-2.37 (m, 1H), 2.17-2.05 (m, 5H), 1.94-1.89 (m, 1H), 1.63-1.62 (m, 1H) MS m/z=538.2 (M+H)+

### Example 28: (S)-1-(2-((2-(quinoxalin-6-yl)pyrimidin-4-yl)amino)-5-(1-(tetrahydro-2H-pyran-4-yl)-1H-pyrazol-4-yl)pyridin-4-yl)piperidin-3-ol

The titled compound (6.9 mg) was prepared in accordance with the same procedures as in Example 22, changing the boronic acid reagent to quinoxalin-6-ylboronic acid reagent. (Yield: 28.6%) 1H NMR (CD₃OD, 400 MHz) δ 8.89 (d, 2H), 8.66 (s, 1H), 8.62 (s, 1H), 8.58-8.56 (m, 1H), 8.26 (s, 1H), 8.19 (d, 1H), 8.03 (d, 1H), 7.91 (s, 1H), 7.65 (s, 1H), 6.07 (d, 1H), 5.17-5.16 (m, 1H), 5.06 (s, 1H), 4.52-4.48 (m, 1H), 4.19-4.13 (m, 2H), 3.71-3.68 (m, 1H), 3.63-3.57 (m, 2H), 3.12-3.11 (m, 1H), 3.07-3.05 (m, 1H), 3.04-2.96 (m, 1H), 2.43-2.39 (m, 1H), 2.18-2.09 (m, 5H), 1.93-1.90 (m, 1H), 1.88-1.85 (m, 1H) MS m/z=550.2 (M+H)+

### Example 29: (S)-1-(2-((2-(8-fluoroquinolin-7-yl)pyrimidin-4-yl)amino)-5-(1-(tetrahydro-2H-pyran-4-yl)-1H-pyrazol-4-yl)pyridin-4-yl)piperidin-3-ol

The titled compound (6.6 mg) was prepared in accordance with the same procedures as in Example 22, changing the boronic acid reagent to 8-fluoro-7-(4,4,5,5-tetramethyl-1,3,2-dioxaborolan-2-yl)quinoline. (Yield: 26.5%) 1H NMR (CD₃OD, 400 MHz) δ 9.02-9.01 (m, 1H), 8.63 (s, 1H), 8.3 (s, 1H), 8.22-8.20 (m, 2H), 7.98 (d, 1H), 7.9 (s, 1H), 7.72-7.70 (m, 2H), 7.52-7.51 (m, 1H), 6.04 (d, 1H), 5.13-5.11 (m, 1H), 5.05 (s, 1H), 4.52-4.48 (m, 1H), 4.19-4.13 (m, 2H), 3.65-3.56 (m, 3H), 3.10-3.00 (m, 1H), 2.99-2.96 (m, 2H), 2.52-2.47 (m, 1H), 2.18-2.04 (m, 4H), 1.85-1.83 (m, 1H), 1.67-1.66 (m, 1H), 1.64-1.61 (m, 2H) MS m/z=567.3 (M+H)+

### Example 30: (S)-1-(6-((2-(2-fluoro-6-methoxyphenyl)pyrimidin-4-yl)amino)-6′-((4-methylpiperazin-1-yl)methyl)-[3,3′-bipyridin]-4-yl)piperidin-3-ol

The titled compound (7.2 mg) was prepared in accordance with the same procedures as in Example 7, using Intermediates A4-2 and C1-1. (Yield: 20.8%) 1H NMR (CDCl₃, 400 MHz) δ 8.74 (s, 1H), 8.61 (d, 1H), 8.01 (s, 1H), 7.80 (d, 1H), 7.56 (s, 1H), 7.45-7.43 (m, 2H), 7.39-7.36 (m, 2H), 6.84-6.80 (m, 2H), 3.89 (s, 3H), 3.71 (s, 2H), 3.66-3.61 (m, 1H), 3.06 (d, 1H), 3.01-2.97 (m, 1H), 2.68-2.50 (m, 9H), 2.01 (s, 3H), 1.83-1.62 (m, 9H) MS m/z=586.3 (M+H)+

### Example 31: (S)-1-(6-((2-(2-fluoro-6-methoxyphenyl)pyrimidin-4-yl)amino)-6′-(tetrahydro-2H-pyran-4-yl)-[3,3′-bipyridin]-4-yl)piperidin-3-ol

The titled compound (8.5 mg) was prepared in accordance with the same procedures as in Example 7, using Intermediates A4-1 and C1-1. (Yield: 19.0%) 1H NMR (CDCl₃, 400 MHz) δ 8.71 (s, 1H), 8.58 (d, 1H), 7.99 (s, 2H), 7.81 (d, 1H), 7.43 (s, 1H), 7.38-7.31 (m, 2H), 7.27-7.23 (m, 2H), 6.83-6.79 (m, 2H), 4.14-4.11 (m, 2H), 3.82 (s, 3H), 3.70-3.69 (m, 1H), 3.60-3.55 (m, 2H), 3.19-3.16 (m, 1H), 3.10-3.00 (m, 1H), 2.98-2.82 (m, 1H), 2.67-2.62 (m, 2H), 1.95-1.93 (m, 4H), 1.92-1.91 (m, 1H), 1.41-1.39 (m, 1H), 1.31-1.27 (m, 2H) MS m/z=557.2 (M+H)+

### Example 32: (S)-1-(6-((2-(2-fluoro-6-methoxyphenyl)pyrimidin-4-yl)amino)-6′-(morpholinomethyl)-[3,3′-bipyridin]-4-yl)piperidin-3-ol

The titled compound (1.7 mg) was prepared in accordance with the same procedures as in Example 7, using Intermediates A4-3 and C1-1. (Yield: 5.01%) 1H NMR (CDCl₃, 400 MHz) δ 8.74 (s, 1H), 8.61 (d, 1H), 8.00 (s, 1H), 7.83 (d, 1H), 7.55-7.53 (m, 1H), 7.49 (d, 1H), 7.43-7.35 (m, 3H), 6.84-6.80 (m, 2H), 3.83 (s, 3H), 3.78-3.76 (m, 4H), 3.72 (s, 3H), 3.18-3.15 (m, 1H), 2.95-2.92 (m, 1H), 2.75-2.65 (m, 3H), 2.01 (s, 4H), 1.86-1.85 (m, 1H), 1.41-1.40 (m, 1H) MS m/z=573.3 (M+H)+

### Example 33: (S)-1-(6-((2-(2-fluoro-6-methoxyphenyl)pyrimidin-4-yl)amino)-6′-((4-hydroxypiperidin-1-yl)methyl)-[3,3′-bipyridin]-4-yl)piperidin-3-ol

The titled compound (1.1 mg) was prepared in accordance with the same procedures as in Example 7, using Intermediates A4-4 and C1-1. (Yield: 3.17%) 1H NMR (CDCl₃, 400 MHz) δ 8.74 (s, 1H), 8.61 (d, 1H), 8.00 (s, 1H), 7.84-7.81 (m, 1H), 7.53-7.35 (m, 5H), 6.82-6.80 (m, 2H), 3.83 (s, 3H), 3.74-3.73 (m, 4H), 3.18-3.12 (m, 1H), 2.97-2.94 (m, 1H), 2.88-2.83 (m, 1H), 2.73-2.60 (m, 4H), 2.38-2.30 (m, 2H), 1.94-1.86 (m, 4H), 1.49-1.40 (m, 3H). MS m/z=587.3 (M+H)+

### Example 34: 1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-carboxamide

The titled compound (8.8 mg) was prepared in accordance with the same procedures as in Example 7, using Intermediates B3-6 and C1-1. (Yield: 32.6%) 1H NMR (CD₃OD, 400 MHz) δ 8.41 (d, 1H), 8.09 (s, 1H), 8.04 (s 1H), 7.84 (s, 1H), 7.60 (s, 1H), 7.51 (d, 1H), 7.44 (q, 1H), 6.96 (d, 1H), 6.85 (t, 1H), 4.49-4.44 (m, 1H), 4.09 (d, 2H), 3.82 (s, 3H), 3.60 (t, 2H), 3.32-3.20 (m, 2H), 2.69 (t, 1H), 2.57-2.52 (m, 2H), 2.15-2.09 (m, 4H), 2.09-1.94 (m, 1H), 1.66-1.55 (m, 3H) MS m/z=573.3 (M+H)+

### Example 35: (S)-1-(2-((6-(2-fluoro-6-methoxyphenyl)pyridin-2-yl)amino)-5-(1-(tetrahydro-2H-pyran-4-yl)-1H-pyrazol-4-yl)pyridin-4-yl)piperidin-3-ol

The titled compound (4.4 mg) was prepared in accordance with the same procedures as in Example 7, using Intermediates A2-1 and C1-8. (Yield: 22.9%) 1H NMR (CDCl₃, 400 MHz) δ 8.05 (s, 1H), 7.74 (s, 2H), 7.68-7.66 (m, 1H), 7.57-7.53 (m, 1H), 7.34-7.33 (m, 1H), 7.01 (s, 1H), 6.85-6.80 (m, 2H), 4.41-4.37 (m, 1H), 3.83 (d, 2H), 3.81 (s, 3H), 3.58 (t, 2H), 3.24-3.21 (m, 1H), 2.98-2.95 (m, 1H), 2.73-2.70 (m, 2H), 2.16-2.06 (m, 5H), 1.94-1.88 (m, 1H), 1.78-1.74 (m, 1H), 1.52-1.49 (m, 2H). MS m/z=545.4 (M+H)+

### Example 36: (S)-(6′-((2-(2-fluoro-6-methoxyphenyl)pyrimidin-4-yl)amino)-4′-(3-hydroxypiperidin-1-yl)-[3,3′-bipyridin]-6-yl)(4-methylpiperazin-1-yl)methanone

The titled compound (2.5 mg) was prepared in accordance with the same procedures as in Example 7, using Intermediates A4-5 and C1-1. (Yield: 7.63%) 1H NMR (CDCl₃, 400 MHz) δ 8.78 (s, 1H), 8.62 (d, 1H), 8.00-7.96 (m, 2H), 7.75 (d, 1H), 7.61-7.59 (m, 1H), 7.53-7.50 (m, 1H), 7.40-7.34 (m, 2H), 6.85-6.81 (m, 2H), 3.90-3.88 (m, 2H), 3.81 (s, 3H), 3.75-3.72 (m, 3H), 3.03-3.02 (m, 2H), 2.68-2.65 (m, 2H), 2.58-2.56 (m, 2H), 2.52-2.49 (m, 2H), 2.20 (s, 3H), 2.06-2.01 (m, 2H), 1.43-1.41 (m, 2H). MS m/z=599.2 (M+H)+

### Example 37: (S)-(6′-((2-(2-fluoro-6-methoxyphenyl)pyrimidin-4-yl)amino)-4′-(3-hydroxypiperidin-1-yl)-[3,3′-bipyridin]-6-yl)(morpholino)methanone

The titled compound (1.9 mg) was prepared in accordance with the same procedures as in Example 7, using Intermediates A4-6 and C1-1. (Yield: 5.93%) 1H NMR (CDCl₃, 400 MHz) δ 8.78 (s, 1H), 8.62 (d, 1H), 8.00-7.98 (m, 2H), 7.75 (d, 1H), 7.53-7.49 (m, 2H), 7.40-7.34 (m, 2H), 6.85-6.81 (m, 2H), 3.86-3.82 (m, 7H), 3.79-3.70 (m, 6H), 3.18-3.16 (m, 1H), 2.98-2.94 (m, 1H), 2.68-2.63 (m, 2H), 2.03-2.00 (m, 1H), 1.91-1.88 (m, 1H), 1.43-1.41 (m, 2H). MS m/z=586.2 (M+H)+

### Example 38: (S)-(6′-((2-(2-fluoro-6-methoxyphenyl)pyrimidin-4-yl)amino)-4′-(3-hydroxypiperidin-1-yl)-[3,3′-bipyridin]-6-yl)(4-hydroxypiperidin-1-yl)methanone

The titled compound (2.0 mg) was prepared in accordance with the same procedures as in Example 7, using Intermediates A4-7 and C1-1. (Yield: 7.31%) 1H NMR (CD₃OD), 400 MHz) δ 8.81 (s, 1H), 8.45 (d, 1H), 8.16 (d, 1H), 8.05 (s, 1H), 7.70-7.66 (m, 2H), 7.57 (d, 1H), 7.46-7.42 (m, 1H), 6.97 (d, 1H), 6.85 (t, 1H), 4.21-4.19 (m, 1H), 3.83 (s, 3H), 3.74-3.71 (m, 1H), 3.60-3.56 (m, 1H), 3.55-3.53 (m, 1H), 3.49-3.40 (m, 1H), 3.19-3.16 (m, 1H), 3.08-3.05 (m, 1H), 2.65-2.60 (m, 1H), 2.46-2.41 (m, 1H), 2.06-1.98 (m, 3H), 1.95-1.89 (m, 2H), 1.63-1.50 (m, 4H). MS m/z=600.3 (M+H)+

### Example 39: (S)-1-(6-((2-(2-fluoro-6-methoxyphenyl)pyrimidin-4-yl)amino)-6′-(((S)-3-fluoropyrrolidin-1-yl)methyl)-[3,3′-bipyridin]-4-yl)piperidin-3-ol

The titled compound (2.8 mg) was prepared in accordance with the same procedures as in Example 7, using Intermediates A8-1 and C1-1. (Yield: 7.13%) 1H NMR (CD₃OD, 400 MHz) δ 8.74 (s, 1H), 8.61 (d, 1H), 8.00 (s, 1H), 7.83 (d, 1H), 7.55-7.53 (m, 1H), 7.49 (d, 1H), 7.43-7.35 (m, 3H), 6.84-6.80 (m, 2H), 3.83 (s, 3H), 3.78-3.76 (m, 4H), 3.72 (s, 3H), 3.18-3.15 (m, 1H), 2.95-2.92 (m, 1H), 2.75-2.65 (m, 3H), 2.01 (s, 4H), 1.86-1.85 (m, 1H), 1.41-1.40 (m, 8H). MS m/z=574.3 (M+H)+

### Example 40: (3S)-1-(2-((5-fluoro-6-(2-fluoro-6-methoxyphenyl)pyridin-2-yl)amino)-5-(1-(tetrahydro-2H-pyran-4-yl)-1H-pyrazol-4-yl)pyridin-4-yl)piperidin-3-ol

The titled compound (8.2 mg) was prepared in accordance with the same procedures as in Example 7, using Intermediates A2-1 and C1-2. (Yield: 28.2%) 1H NMR (CDCl₃, 400 MHz) δ 8.69 (s, 1H), 8.44 (d, 1H), 8.04 (d, 1H), 8.00 (s, 1H), 7.67-7.55 (m, 3H), 7.57 (d, 1H), 6.97 (d, 1H), 6.97 (d, 1H), 6.85 (t, 1H), 3.87 (s, 2H), 3.83 (s, 3H), 3.54-3.49 (m, 2H), 3.22-3.14 (m, 1H), 3.07-3.02 (m, 1H), 2.99-2.93 (m, 2H), 2.87-2.75 (m, 1H), 2.62-2.53 (m, 2H), 2.44-2.39 (m, 1H), 2.08-2.02 (m, 2H), 1.93-1.90 (m, 1H), 1.61-1.58 (m, 2H), 1.45-1.42 (m, 1H). MS m/z=563.4 (M+H)+

### Example 41: (3S)-1-(2-((6-(2-fluoro-6-methoxyphenyl)-5-nitropyridin-2-yl)amino)-5-(1-(tetrahydro-2H-pyran-4-yl)-1H-pyrazol-4-yl)pyridin-4-yl)piperidin-3-ol

The titled compound (6.3 mg) was prepared in accordance with the same procedures as in Example 7, using Intermediates A2-1 and C3-1. (Yield: 20.7%) 1H NMR (CDCl₃, 400 MHz) δ 8.38 (d, 1H), 8.11 (s, 1H), 7.78 (s, 2H), 7.69-7.68 (m, 1H), 7.53-7.45 (m, 2H), 7.41-7.36 (m, 1H), 6.89-6.84 (m, 1H), 6.77 (d, 1H), 4.44-4.37 (m, 1H), 4.16-4.11 (m, 3H), 3.85-3.79 (m, 1H), 3.75 (s, 3H), 3.61-3.56 (m, 3H), 3.20-3.14 (m, 1H), 2.97-2.89 (m, 1H), 2.20 (s, 2H), 2.16-2.04 (m, 4H), 1.90-1.87 (m, 1H), 1.75-1.70 (m, 1H). MS m/z=590.2 (M+H)+

### Example 42: (S)-1-(6-((2-(2-fluoro-6-methoxyphenyl)pyrimidin-4-yl)amino)-[3,3′-bipyridin]-4-yl)piperidin-3-ol

The titled compound (2.3 mg) was prepared in accordance with the same procedures as in Example 7, using Intermediates A2-15 and C1-1. (Yield: 4.3%) 1H NMR (CDCl₃, 400 MHz) δ 8.78 (s, 1H), 8.60-8.57 (m, 2H), 7.99 (s, 1H), 7.87-7.85 (m, 1H), 7.81 (s, 1H), 7.45 (s, 1H), 7.40-7.27 (m, 2H), 6.84-6.80 (m, 2H), 3.83 (s, 3H), 3.70-3.62 (m, 1H), 3.19-3.16 (m, 1H), 2.95-2.92 (m, 1H), 2.67-2.62 (m, 2H), 1.85-1.74 (m, 2H), 1.60-1.52 (m, 11H), 1.44-1.31 (m, 2H) MS m/z=473.2 (M+H)+

### Example 43: (3S)-1-(6-((2-(2-fluoro-6-methoxyphenyl)pyrimidin-4-yl)amino)-6′-((3-hydroxypyrrolidin-1-yl)methyl)-[3,3′-bipyridin]-4-yl)piperidin-3-ol

The titled compound (6.2 mg) was prepared in accordance with the same procedures as in Example 7, using Intermediates A8-2 and C1-1. (Yield: 15.9%) 1H NMR (CD₃OD, 400 MHz) δ 8.69 (s, 1H), 8.44 (d, 1H), 8.03 (d, 1H), 8.00 (s, 1H), 7.67-7.55 (m, 3H), 7.46-7.42 (m, 1H), 6.97 (d, 1H), 6.97 (d, 1H), 6.85 (t, 1H), 3.82 (s, 3H), 3.54-3.49 (m, 1H), 3.22-3.19 (m, 1H), 3.07-3.04 (m, 1H), 2.89-2.85 (m, 2H), 2.62-2.56 (m, 3H), 2.44-2.39 (m, 1H), 2.21-2.14 (m, 2H), 2.04-2.02 (m, 1H), 1.92-1.90 (m, 2H), 1.77-1.72 (m, 1H), 1.62-1.60 (m, 1H), 1.46-1.43 (m, 1H). MS m/z=572.2 (M+H)+

### Example 44: N-(4-(3-(dimethylamino)pyrrolidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine

The titled compound (6.2 mg) was prepared in accordance with the same procedures as in Example 7, using Intermediates B3-1 and C1-1. (Yield: 17.1%) 1H NMR (CD₃OD, 400 MHz) δ 8.39 (d, 1H), 7.79 (s, 1H), 7.76 (s 1H), 7.53 (s, 1H), 7.46-7.35 (m, 3H), 6.96 (d, 1H), 6.83 (t, 1H), 4.48-4.41 (m, 1H), 4.08 (d, 2H), 3.81 (s, 3H), 3.59 (t, 2H), 3.19-3.13 (m, 3H), 2.91 (t, 1H), 2.72-2.63 (m, 1H), 2.16 (s, 6H), 2.14-2.02 (m, 4H), 1.73-1.65 (m, 1H), 1.41-1.29 (m, 1H) MS m/z=559.2 (M+H)+

### Example 45: (1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)-3-methylpyrrolidin-3-yl)methanol

The titled compound (12.6 mg) was prepared in accordance with the same procedures as in Example 7, using Intermediates B3-2 and C1-1. (Yield: 34.3%) 1H NMR (CD₃OD, 400 MHz) δ 8.40 (d, 1H), 7.77 (s, 1H), 7.75 (s 1H), 7.51 (s, 1H), 7.43 (q, 1H), 7.39 (s, 1H), 7.34 (d, 1H), 6.96 (d, 1H), 6.84 (t, 1H), 4.46-4.43 (m, 1H), 4.08 (d, 2H), 3.82 (s, 3H), 3.59 (t, 2H), 3.35-3.31 (m, 2H), 3.14-3.08 (m, 2H), 3.03 (dd, 1H), 2.74 (dd, 1H), 2.13-2.07 (m, 4H), 1.81-1.77 (m, 1H), 1.56-1.51 (m, 1H) MS m/z=560.2 (M+H)+

### Example 46: N-(4-(4-((dimethylamino)methyl)-4-fluoropiperidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine

The titled compound (2.2 mg) was prepared in accordance with the same procedures as in Example 7, using Intermediates B3-3 and C1-1. (Yield: 7.7%) 1H NMR (CD₃OD, 400 MHz) δ 8.41 (d, 1H), 8.09 (s, 1H), 8.06 (s 1H), 7.86 (s, 1H), 7.77 (s, 1H), 7.48-7.42 (m, 2H), 6.96 (d, 1H), 6.85 (t, 1H), 4.50-4.44 (m, 1H), 4.09 (d, 2H), 3.83 (s, 3H), 3.60 (t, 2H), 3.13 (d, 2H), 2.85 (t, 2H), 2.54 (s, 1H), 2.48 (s, 1H), 2.31 (s, 6H), 2.15-2.06 (m, 4H), 1.87-1.77 (m, 1H), 1.75-1.69 (m, 1H) MS m/z=606.3 (M+H)+

### Example 47: (4-(2,2-difluoroethyl)-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-4-yl)methanol

The titled compound (14.1 mg) was prepared in accordance with the same procedures as in Example 7, using Intermediates B3-4 and C1-1. (Yield: 53.5%) 1H NMR (CD₃OD, 400 MHz) δ 8.41 (d, 1H), 8.08 (s, 1H), 8.07 (s 1H), 7.85 (s, 1H), 7.74 (s, 1H), 7.47-7.41 (m, 2H), 6.96 (d, 1H), 6.84 (t, 1H), 6.12 (t, 1H), 4.50-4.45 (m, 1H), 4.09 (d, 2H), 3.82 (s, 3H), 3.60 (t, 2H), 3.50 (s, 2H), 2.99-2.94 (m, 2H), 2.90-2.84 (m, 2H), 2.15-2.09 (m, 4H), 2.00-1.89 (m, 2H), 1.62-1.46 (m, 4H) MS m/z=624.3 (M+H)+

### Example 48: (3S)-1-(6′-((3-(dimethylamino)pyrrolidin-1-yl)methyl)-6-((2-(2-fluoro-6-methoxyphenyl)pyrimidin-4-yl)amino)-[3,3′-bipyridin]-4-yl)piperidin-3-ol

The titled compound (5.8 mg) was prepared in accordance with the same procedures as in Example 7, using Intermediates A8-3 and C1-1. (Yield: 16.3%) 1H NMR (CD₃OD, 400 MHz) δ 8.69 (s, 1H), 8.44 (d, 1H), 8.03 (d, 1H), 8.00 (s, 1H), 7.67 (s, 1H), 7.60-7.55 (m, 2H), 7.46-7.42 (m, 1H), 6.97 (d, 1H), 6.85 (t, 1H), 3.89-3.77 (m, 5H), 3.53-3.49 (m, 1H), 3.21-3.18 (m, 1H), 3.07-3.04 (m, 1H), 2.95-2.91 (m, 2H), 2.83-2.79 (m, 1H), 2.68-2.56 (m, 2H), 2.47-2.40 (m, 2H), 2.25 (s, 6H), 2.08-2.02 (m, 1H), 1.92-1.90 (m, 1H), 1.78-1.73 (m, 1H), 1.63-1.60 (m, 1H), 1.46-1.42 (m, 1H). MS m/z=599.3 (M+H)+

### Example 49: 1-((6′-((2-(2-fluoro-6-methoxyphenyl)pyrimidin-4-yl)amino)-4′-((S)-3-hydroxypiperidin-1-yl)-[3,3′-bipyridin]-6-yl)methyl)piperidin-3-ol

The titled compound (2.4 mg) was prepared in accordance with the same procedures as in Example 7, using Intermediates A8-4 and C1-1. (Yield: 6.91%) 1H NMR (CD₃OD, 400 MHz) δ 8.69 (s, 1H), 8.44 (d, 1H), 8.04-8.00 (m, 2H), 7.67-7.55 (m, 3H), 7.48-7.42 (m, 1H), 7.57 (d, 1H), 6.85 (t, 1H), 3.83 (s, 3H), 3.74-3.67 (m, 4H), 3.60-3.49 (m, 2H), 3.21-3.18 (m, 1H), 3.07-3.03 (m, 1H), 2.92-2.87 (m, 1H), 2.72-2.71 (m, 1H), 2.61-2.56 (m, 1H), 2.44-2.39 (m, 1H), 2.19-2.16 (m, 1H), 2.25 (s, 1H), 1.96-1.91 (m, 2H), 1.81-1.76 (m, 1H), 1.61-1.54 (m, 2H), 1.43-1.41 (m, 1H). MS m/z=586.2 (M+H)+

### Example 50: (S)-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-carboxamide

The titled compound (2.1 mg) was prepared in accordance with the same procedures as in Example 7, using Intermediates B3-5 and C1-1. (Yield; 12.5%) 1H NMR (CD₃OD, 400 MHz) δ 8.41 (d, 1H), 8.09 (s, 1H), 8.04 (s 1H), 7.84 (s, 1H), 7.60 (s, 1H), 7.52-7.43 (m, 2H), 6.96 (d, 1H), 6.86 (t, 1H), 4.54-4.40 (m, 1H), 4.09 (d, 2H), 3.82 (s, 3H), 3.63 (t, 2H), 3.32-3.14 (m, 2H), 2.69 (t, 1H), 2.55-2.53 (m, 2H), 2.15-2.09 (m, 4H), 2.09-1.94 (m, 11H), 1.66-1.56 (m, 3H) MS m/z=573.3 (M+H)+

### Example 51: (R)-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-carboxamide

The titled compound (2.1 mg) was prepared in accordance with the same procedures as in Example 7, using Intermediates B3-6 and C1-1. (Yield: 10.8%) 1H NMR (CD₃OD, 400 MHz) δ 8.41 (d, 1H), 8.09 (s, 1H), 8.04 (s 1H), 7.84 (s, 1H), 7.60 (s, 1H), 7.52-7.41 (m, 2H), 6.96 (d, 1H), 6.85 (t, 1H), 4.56-4.40 (m, 1H), 4.09 (d, 2H), 3.82 (s, 3H), 3.63 (t, 2H), 3.32-3.20 (m, 2H), 2.69 (t, 1H), 2.60-2.48 (m, 2H), 2.15-2.02 (m, 4H), 2.02-1.95 (m, 1H), 1.66-1.55 (m, 3H) MS m/z=573.3 (M+H)+

### Example 52: (S)-N-(4-(3-aminopiperidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine hydrochloride

### Step 1: tert-butyl (S)-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-yl)carbamate

tert-Butyl (S)-(1-(2-chloro-5-(1-(tetrahydro-2H-pyran-4-yl)-1H-pyrazol-4-yl)pyridin-4-yl)piperidin-3-yl)carbamate (intermediate A3-1, 100 mg, 0.21 mmol) was dissolved in anhydrous 1,4-dioxane (4.0 mL) and then 2-(2-fluoro-6-methoxyphenyl)pyrimidin-4-amine (C1-1, 47.4 mg, 0.21 mmol), tris(dibenzylideneacetone)dipalladium(0) (59.4 mg, 0.065 mmol), 2-dicyclohexylphosphino-2′,4′,6′-triisopropylbiphenyl (61.9 mg, 0.13 mmol), and cesium carbonate (211.6 mg, 0.65 mmol) were added dropwise thereto. Then, the mixed solution was refluxed under stirring at 100 °C overnight. After completion of the reaction, the reaction mixture was cooled to room temperature and water was added thereto. The aqueous layer was extracted with ethyl acetate. The extracted organic layer was dried over anhydrous magnesium sulfate and then filtered. The filtrate was concentrated and then purified by silica gel column chromatography to prepare the titled compound (75.8 mg). (Yield: 54.3%) 1H NMR (CDCl₃, 400 MHz) δ 8.58 (d, 1H), 8.11 (s, 1H), 7.79-7.76 (m, 2H), 7.41 (s, 1H), 7.38-7.33 (m, 3H), 6.85-6.80 (m, 2H), 4.48-4.38 (m, 2H), 4.16-4.13 (m, 2H), 3.82 (s, 3H), 3.74-3.73 (m, 1H), 3.71-3.55 (m, 2H), 3.37-3.35 (m, 1H), 3.05-3.03 (m, 1H), 2.56-2.47 (m, 2H), 2.16-2.11 (m, 4H), 1.95-1.93 (m, 1H), 1.57-1.54 (m, 1H), 1.52 (s, 9H), 1.45-1.32 (m, 1H)

### Step 2: (S)-N-(4-(3-aminopiperidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine hydrochloride

tert-Butyl (S)-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-yl)carbamate (75.0 mg, 0.12 mmol) was dissolved in dichloromethane (5.0 ml) and then 4.0 M hydrochloric acid solution dissolved in 1,4-dioxane (1.5 ml, 7.69 mmol) was added dropwise thereto. Then, the mixed solution was stirred at room temperature for 4 hours. After completion of the reaction, the reaction mixture was concentrated and the resulting solid was dried to prepare the titled compound (60.0 mg). (Yield: 82.3%) 1H NMR (CD₃OD, 400 MHz) δ 8.72 (d, 1H), 8.16 (s, 1H), 8.13 (s 1H), 7.85 (s, 1H), 7.65-7.40 (m, 2H), 7.14-7.09 (m, 2H), 6.97 (t, 1H), 4.54-4.50 (m, 1H), 4.10 (d, 2H), 3.92 (s, 3H), 3.81 (d, 1H), 3.60 (t, 2H), 3.58-3.32 (m, 2H), 2.95-2.85 (m, 2H), 2.21-2.11 (m, 5H), 1.86-1.58 (m, 3H) MS m/z=545.4 (M+H)+

### Example 53: (R)-N-(4-(3-aminopiperidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine

The titled compound (4.1 mg) was prepared in accordance with the same procedures as in Example 52, using Intermediates B3-7 and C1-1. (Yield: 70.0%) 1H NMR (CD₃OD, 400 MHz) δ 8.76 (d, 1H), 8.20-8.10 (m, 2H), 7.45 (s, 1H), 7.66 (q, 1H), 7.54 (s, 1H), 7.22 (s, 1H), 7.13 (d, 1H), 7.00 (t, 1H), 4.54-4.51 (m, 1H), 4.09 (d, 2H), 3.95 (s, 3H), 3.88 (d, 1H), 3.66-3.57 (m, 3H), 3.57-3.32 (m, 1H), 3.04-2.90 (m, 2H), 2.22-2.11 (m, 5H), 1.85-1.58 (m, 3H) MS m/z=545.4 (M+H)+

### Example 54: (6′-((6-(2-fluoro-6-methoxyphenyl)-5-nitropyridin-2-yl)amino)-4′-((S)-3-hydroxypiperidin-1-yl)-[3,3′-bipyridin]-6-yl)(4-hydroxypiperidin-1-yl)methanone

The titled compound (2.5 mg) was prepared in accordance with the same procedures as in Example 7, using Intermediates A4-7 and C3-1. (Yield: 6.48%) 1H NMR (CD₃OD, 400 MHz) δ 8.74 (s, 1H), 8.57 (d, 1H), 8.09-8.08 (m, 1H), 7.99 (s, 1H), 7.71 (d, 1H), 7.55-7.49 (m, 1H), 7.28 (d, 1H), 4.18-4.20 (m, 1H), 3.94-3.91 (m, 1H), 3.79 (s, 3H), 3.69-3.67 (m, 3H), 3.49-3.41 (m, 2H), 3.14-3.12 (m, 1H), 2.97-2.91 (m, 1H), 1.89-1.80 (m, 3H), 1.51-1.47 (m, 6H) MS m/z=644.3 (M+H)+

### Example 55: (S)-1-(6′-((4-aminopiperidin-1-yl)methyl)-6-((2-(2-fluoro-6-methoxyphenyl)pyrimidin-4-yl)amino)-[3,3′-bipyridin]-4-yl)piperidin-3-ol hydrochloride

The titled compound (1.3 mg) was prepared in accordance with the same procedures as in Example 52, using Intermediates A8-5 and C1-1. (Yield: 13.5%) 1H NMR (CD₃OD, 400 MHz) δ 8.85 (s, 1H), 8.72 (d, 1H), 8.09-8.06 (m, 2H), 7.71 (d, 1H), 7.62-7.56 (m, 1H), 7.38-7.30 (m, 1H), 7.10-7.07 (m, 2H), 6.95 (t, 1H), 3.90 (s, 3H), 3.79-3.76 (m, 2H), 3.68-3.65 (m, 1H), 3.75 (s, 1H), 3.40-3.38 (m, 3H), 3.18-3.05 (m, 1H), 2.92-2.86 (m, 1H), 2.20 (s, 2H), 2.16-2.11 (m, 3H), 1.93-1.83 (m, 2H), 1.55-1.50 (m, 3H). MS m/z=585.3 (M+H)+

### Example 56: (S)-1-(6′-ethoxy-6-((2-(2-fluoro-6-methoxyphenyl)pyrimidin-4-yl)amino)-[3,3′-bipyridin]-4-yl)piperidin-3-ol

The titled compound (1.4 mg) was prepared in accordance with the same procedures as in Example 7, using Intermediates A2-16 and C1-1. (Yield: 2.4%) 1H NMR (CDCl₃, 400 MHz) δ 8.43 (d, 1H), 8.26 (s, 1H), 7.94 (s, 1H), 7.93 (d, 1H), 7.62 (s, 1H), 7.55-7.53 (m, 1H), 7.45-7.43 (m, 1H), 6.97 (d, 1H), 6.87-6.82 (m, 2H), 4.38 (q, 2H), 3.82 (s, 3H), 3.57-3.53 (m, 1H), 3.27-3.24 (m, 1H), 3.09-3.06 (m, 1H), 2.60-2.55 (m, 1H), 2.42-2.37 (m, 1H), 1.64-1.61 (m, 1H), 1.47-1.42 (m, 1H), 1.39 (t, 3H) MS m/z=503.1 (M+H)+

### Example 57: (S)-1-(6-((2-(2-fluoro-6-methoxyphenyl)pyrimidin-4-yl)amino)-6′-(trifluoromethyl)-[3,3′-bipyridin]-4-yl)piperidin-3-ol

The titled compound (9.2 mg) was prepared in accordance with the same procedures as in Example 7, using Intermediates A2-17 and C1-1. (Yield: 14.9%) 1H NMR (CD₃OD, 400 MHz) δ 8.93 (s, 1H), 8.45 (d, 1H), 8.27 (d, 1H), 8.05 (s, 1H), 7.89 (d, 1H), 7.7 (s, 1H), 7.58-7.56 (m, 1H), 7.45-7.43 (m, 1H), 6.97 (d, 1H), 6.85 (dd, 1H), 3.82 (s, 3H), 3.56-3.31 (m, 1H), 3.18-3.17 (m, 1H), 3.04-3.01 (m, 1H), 2.64-2.62 (m, 1H), 2.48-2.43 (m, 1H), 1.92-1.88 (m, 1H), 1.65-1.60 (m, 1H), 1.44-1.41 (m, 1H), 1.31-1.28 (m, 1H) MS m/z=541.2 (M+H)+

### Example 58: (3S)-1-(2-((6-(2-fluoro-6-methoxyphenyl)-5-methylpyridin-2-yl)amino)-5-(1-(tetrahydro-2H-pyran-4-yl)-1H-pyrazol-4-yl)pyridin-4-yl)piperidin-3-ol

The titled compound (7.4 mg) was prepared in accordance with the same procedures as in Example 7, using Intermediates A2-1 and C1-4. (Yield: 23.6%) 1H NMR (CDCl₃, 400 MHz) δ 7.75-7.55 (m, 3H), 7.53-7.35 (m, 3H), 7.22-7.15 (m, 1H), 6.87-6.82 (m, 2H), 4.42-4.36 (m, 1H), 4.15-4.12 (m, 2H), 3.93-3.87 (m, 1H), 3.80 (s, 3H), 3.60-3.54 (m, 3H), 3.17-2.87 (m, 2H), 2.28-1.96 (m, 7H), 1.89-1.86 (m, 2H). MS m/z=559.2 (M+H)+

### Example 59: 2-(2-fluoro-6-methoxyphenyl)-6-((4-((S)-3-hydroxypiperidin-1-yl)-5-(1-(tetrahydro-2H-pyran-4-yl)-1H-pyrazol-4-yl)pyridin-2-yl)amino)nicotinonitrile

The titled compound (1.4 mg) was prepared in accordance with the same procedures as in Example 7, using Intermediates A2-1 and C8-1. (Yield: 4.55%) 1H NMR (CDCl₃, 400 MHz) δ 8.07-8.03 (m, 1H), 7.83 (d, 1H), 7.75 (s, 2H), 7.63-7.53 (m, 1H), 7.45-7.40 (m, 2H), 6.88-6.84 (m, 2H), 4.43-4.38 (m, 1H), 4.15 (d, 2H), 3.88 (s, 3H), 3.84-3.76 (m, 1H), 3.58 (t, 3H), 3.20-3.18 (m, 1H), 2.72-2.70 (m, 1H), 2.18-2.03 (m, 5H), 1.90-1.88 (m, 1H), 1.78-1.73 (m, 1H), 1.52-1.49 (m, 1H) MS m/z=570.2 (M+H)+

### Example 60: methyl 2-(2-fluoro-6-methoxyphenyl)-6-((4-((S)-3-hydroxypiperidin-1-yl)-5-(1-(tetrahydro-2H-pyran-4-yl)-1H-pyrazol-4-yl)pyridin-2-yl)amino)nicotinate

The titled compound (1.5 mg) was prepared in accordance with the same procedures as in Example 7, using Intermediates A2-1 and C2-1. (Yield: 4.52%) 1H NMR (CDCl₃, 400 MHz) δ 8.27 (d, 1H), 8.10-8.05 (m, 1H), 7.74 (s, 2H), 7.64-7.53 (m, 1H), 7.45-7.34 (m, 2H), 6.85-6.76 (m, 2H), 4.43-4.36 (m, 1H), 4.15 (d, 2H), 3.81-3.80 (m, 1H), 3.75 (s, 3H), 3.70 (s, 3H), 3.58 (t, 2H), 3.17-3.10 (m, 1H), 2.97-2.90 (m, 1H), 2.74-2.67 (m, 1H), 2.14-2.03 (m, 5H), 1.89-1.85 (m, 1H), 1.74-1.70 (m, 1H), 1.50-1.46 (m, 2H) MS m/z=603.2 (M+H)+

### Example 61: (S)-1-(2-((2-(2-fluoro-6-methoxyphenyl)pyrimidin-4-yl)amino)-5-(imidazo[1,2-a]pyridin-6-yl)pyridin-4-yl)piperidin-3-ol

The titled compound (2.6 mg) was prepared in accordance with the same procedures as in Example 7, using Intermediates A2-18 and C1-1. (Yield: 5.6%) 1H NMR (CDCl₃, 400 MHz) δ 8.61 (d, 1H), 8.27 (s, 1H), 8.03 (s, 1H), 7.66 (d, 3H), 7.62-7.53 (m, 1H), 7.45-7.33 (m, 4H), 6.85-6.80 (m, 2H), 3.83 (s, 3H), 3.82-3.75 (m, 1H), 3.28-3.25 (m, 1H), 3.04-3.01 (m, 1H), 2.76-2.69 (m, 2H), 1.92-1.87 (m, 1H), 1.71-1.70 (m, 2H), 1.65-1.58 (m, 2H) MS m/z=512.2 (M+H)+

### Example 62: (S)-1-(5-([1,2,4]triazolo[4,3-a]pyridin-6-yl)-2-((2-(2-fluoro-6-methoxyphenyl)pyrimidin-4-yl)amino)pyridin-4-yl)piperidin-3-ol

The titled compound (2.0 mg) was prepared in accordance with the same procedures as in Example 7, using Intermediates A2-19 and C1-1. (Yield: 4.2%) 1H NMR (CDCl₃, 400 MHz) δ 8.84 (s, 1H), 8.62 (d, 1H), 8.26 (s, 1H), 8.02 (s, 1H), 7.81-7.79 (m, 1H), 7.56-7.54 (m, 2H), 7.40-7.34 (m, 2H), 6.85-6.81 (m, 2H), 3.83 (s, 3H), 3.75-3.62 (m, 1H), 3.28-3.25 (m, 1H), 3.12-3.02 (m, 1H), 2.79-2.70 (m, 2H), 2.05-2.03 (m, 1H), 1.90-1.87 (m, 2H), 1.69-1.59 (m, 2H) MS m/z=513.2 (M+H)+

### Example 63: 2-(2-fluoro-6-methoxyphenyl)-6-((4-((S)-3-hydroxypiperidin-1-yl)-5-(1-(1-methylpiperidin-4-yl)-1H-pyrazol-4-yl)pyrimidin-2-yl)amino)nicotinonitrile

The titled compound (7.1 mg) was prepared in accordance with the same procedures as in Example 7, using Intermediates A9-1 and C8-1. (Yield: 22.9%) 1H NMR (CDCl₃, 400 MHz) 8.57 (d, 1H), 8.08 (s, 1H), 8.04 (s, 1H), 7.99 (d, 1H), 7.61 (d, 2H), 7.41-7.39 (m, 1H), 6.86-6.82 (m, 2H), 4.21-4.16 (m, 1H), 3.85 (s, 3H), 3.59-3.55 (m, 1H), 3.34-3.32 (m, 2H), 3.15-3.12 (m, 1H), 3.02-2.99 (m, 2H), 2.35 (s, 3H), 2.23-2.05 (m, 6H), 1.87-1.85 (m, 1H), 1.77-1.76 (m, 1H), 1.62-1.58 (m, 1H), 1.47-1.45 (m, 1H) MS m/z=564.3 (M+H)+

### Example 64: N-(4-(4-(2-(dimethylamino)ethyl)piperidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine

The titled compound (6.7 mg) was prepared in accordance with the same procedures as in Example 7, using Intermediates B3-8 and C1-1. (Yield: 43.6%) 1H NMR (CDCl₃, 400 MHz) δ 8.70 (d, 1H), 7.96-7.84 (m, 1H), 7.63 (s, 1H), 7.62 (s, 1H), 7.53-7.50 (m, 2H), 7.43 (q, 1H), 7.01-6.84 (m, 2H), 4.45-4.40 (m, 1H), 4.16 (d, 2H), 3.86 (s, 3H), 3.69 (d, 2H), 3.59 (t, 2H), 3.10-2.98 (m, 2H), 2.81 (t, 2H), 2.81 (s, 6H), 2.14-2.01 (m, 5H), 1.88-1.85 (m, 2H), 1.75-1.72 (m, 2H), 1.38-1.11 (m, 2H) MS m/z=601.3 (M+H)+

### Example 65: 1-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-4-yl)cyclopropan-1-ol

The titled compound (1.8 mg) was prepared in accordance with the same procedures as in Example 7, using Intermediates B3-9 and C1-1. (Yield: 8.3%) 1H NMR (CD₃OD, 400 MHz) δ 8.44 (d, 1H), 8.08 (s, 1H), 8.05 (s, 1H), 7.83 (s, 1H), 7.60 (s, 1H), 7.47-7.44 (m, 2H), 6.98 (d, 1H), 6.86 (t, 1H), 4.49-4.46 (m, 1H), 4.09 (d, 2H), 3.83 (s, 3H), 3.63-3.57 (m, 6H), 3.38-3.31 (m, 2H), 2.63 (t, 2H), 2.58-2.52 (m, 2H), 2.17-2.02 (m, 5H), 1.80-1.76 (m, 2H), 1.67-1.57 (m, 2H), 1.03 (t, 2H) MS m/z=586.3 (M+H)+

### Example 66: 1-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-4-yl)ethan-1-ol

The titled compound (8.1 mg) was prepared in accordance with the same procedures as in Example 7, using Intermediates B3-10 and C1-1. (Yield: 35.9%) 1H NMR (CDCl 3, 400 MHz) δ 8.58 (d, 1H), 8.07 (s, 1H), 7.81 (s, 1H), 7.77 (s, 1H), 7.52 (d, 1H), 7.35 (q, 1H), 7.23 (s, 1H), 6.83-6.79 (m, 2H), 4.44-4.37 (m, 1H), 4.13 (d, 2H), 3.82 (s, 3H), 3.61-3.55 (m, 3H), 3.45-3.32 (m, 2H), 2.53 (t, 2H), 2.19-2.03 (m, 4H), 1.84-1.81 (m, 1H), 1.80-1.43 (m, 4H), 1.20 (d, 3H) MS m/z=574.2 (M+H)+

### Example 67: 2-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-4-yl)-2-methylpropan-1-ol

The titled compound (7.8 mg) was prepared in accordance with the same procedures as in Example 7, using Intermediates B3-11 and C1-1. (Yield: 28.9%) 1H NMR (CDCl₃, 400 MHz) δ 8.58 (d, 1H), 8.08 (s, 1H), 7.81 (s, 1H), 7.79 (s, 1H), 7.59 (d, 1H), 7.35 (q, 1H), 7.14 (s, 1H), 6.83-6.79 (m, 2H), 4.45-4.38 (m, 1H), 4.14 (d, 2H), 3.82 (s, 3H), 3.58 (t, 2H), 3.42-3.39 (m, 4H), 2.52 (t, 2H), 2.20-2.03 (m, 5H), 1.64-1.60 (m, 2H), 1.44-1.26 (m, 2H), 0.86 (s, 6H) MS m/z=602.3 (M+H)+

### Example 68: 2-(2-fluoro-6-methoxyphenyl)-N-(4-(4-methylpiperazin-1-yl)-5-(1-(tetrahydro-2H-pyran-4-yl)-1H-pyrazol-4-yl)pyridin-2-yl)pyrimidin-4-amine

The titled compound (14.2 mg) was prepared in accordance with the same procedures as in Example 7, using Intermediates B3-12 and C1-1. (Yield: 37.1%) 1H NMR (CDCl₃, 400 MHz) δ 8.58 (d, 1H), 8.06 (s, 1H), 7.80 (s, 1H), 7.74 (s, 1H), 7.65 (s, 1H), 7.64-7.32 (m, 2H), 6.83-6.79 (m, 2H), 4.44-4.38 (m, 1H), 4.15 (d, 2H), 3.82 (s, 3H), 3.59 (t, 2H), 3.20-3.02 (m, 4H), 2.58-2.42 (m, 4H), 2.37 (s, 3H), 2.19-2.03 (m, 4H)) MS m/z=545.3 (M+H)+

### Example 69: (S)-1-(2-((2-(7-fluoroquinoxalin-6-yl)pyrimidin-4-yl)amino)-5-(1-(tetrahydro-2H-pyran-4-yl)-1H-pyrazol-4-yl)pyridin-4-yl)piperidin-3-ol

The titled compound (7.0 mg) was prepared in accordance with the same procedures as in Example 7, using Intermediates A2-1 and C1-6. (Yield: 22.3%) 1H NMR (CDCl₃, 400 MHz) δ 8.97 (d, 1H), 8.93 (s, 1H), 8.85 (s, 1H), 8.6 (d, 1H), 8.13 (s, 2H), 8.07 (s, 1H), 7.93 (d, 1H), 7.82 (d, 2H), 6.91-6.90 (m, 1H), 4.45-4.44 (m, 1H), 4.43-4.39 (m, 2H), 4.16-4.13 (m, 1H), 3.94-3.71 (m, 1H), 3.69-3.55 (m, 2H), 3.20-3.16 (m, 1H), 2.98-2.94 (m, 1H), 2.54-2.49 (m, 1H), 2.16-2.03 (m, 5H), 1.71-1.70 (m, 1H), 1.68-1.54 (m, 1H), 1.51-1.48 (m, 1H) MS m/z=568.3 (M+H)+

### Example 70: 4-((S)-3-aminopiperidin-1-yl)-N-(6-(2-fluoro-6-methoxyphenyl)-5-nitropyridin-2-yl)-5-(1-(tetrahydro-2H-pyran-4-yl)-1H-pyrazol-4-yl)pyridin-2-amine hydrochloride

The titled compound (5.3 mg) was prepared in accordance with the same procedures as in Example 52, using Intermediates A3-1 and C3-1. (Yield: 20.8%) 1H NMR (CD₃OD, 400 MHz) δ 8.58 (d, 1H), 8.09 (s, 1H), 8.02 (s, 1H), 7.82 (s, 1H), 7.55-7.51 (m, 1H), 7.32 (d, 1H), 7.01-6.94 (m, 3H), 4.53-4.49 (m, 1H), 4.09 (d, 2H), 3.83-3.78 (m, 4H), 3.64-3.57 (m, 3H), 3.50-3.48 (m, 1H), 2.94-2.85 (m, 2H), 2.22-2.17 (m, 1H), 2.15-2.05 (m, 3H), 1.83-1.73 (m, 2H), 1.64-1.59 (m, 1H) MS m/z=589.3 (M+H)+

### Example 71: methyl 6-((4-((S)-3-aminopiperidin-1-yl)-5-(1-(tetrahydro-2H-pyran-4-yl)-1H-pyrazol-4-yl)pyridin-2-yl)amino)-2-(2-fluoro-6-methoxyphenyl)nicotinate hydrochloride

The titled compound (3.9 mg) was prepared in accordance with the same procedures as in Example 52, using Intermediates A3-1 and C2-1. (Yield: 15.0%) 1H NMR (CD₃OD, 400 MHz) δ 8.58 (d, 1H), 8.08 (s, 1H), 8.00 (s, 1H), 7.82 (s, 1H), 7.49-7.47 (m, 1H), 7.24 (d, 1H), 6.99-6.88 (m, 3H), 4.54-4.50 (m, 1H), 4.09 (d, 2H), 3.79-3.78 (m, 4H), 3.71 (s, 3H), 3.64-3.60 (m, 2H), 3.57-3.49 (m, 2H), 2.90-2.84 (m, 2H), 2.20-2.04 (m, 5H), 1.85-1.82 (m, 1H), 1.79-1.75 (m, 1H), 1.62-1.57 (m, 1H) MS m/z=602.3 (M+H)+

### Example 72: (S)-1-(5-(1-(difluoromethyl)-1H-pyrazol-4-yl)-2-((2-(2-fluoro-6-methoxyphenyl)pyrimidin-4-yl)amino)pyridin-4-yl)piperidin-3-ol

The titled compound (3.6 mg) was prepared in accordance with the same procedures as in Example 7, using Intermediates A2-8 and C1-1. (Yield: 11.6%) 1H NMR (CDCl₃, 400 MHz) δ 8.60-8.58 (m, 1H), 8.13-8.11 (m, 2H), 7.95 (s, 1H), 7.68 (s, 1H), 7.42-7.32 (m, 3H), 7.25-7.24 (m, 1H), 6.84-6.80 (m, 2H), 4.88 (s, 1H), 3.82 (s, 3H), 3.23-3.20 (m, 1H), 3.00-2.96 (m, 1H), 2.70-2.64 (m, 2H), 1.93-1.91 (m, 1H), 1.86-1.74 (m, 5H) MS m/z=512.2 (M+H)+

### Example 73: (S)-N-(4-(3-aminopiperidin-1-yl)-5-(1-(difluoromethyl)-1H-pyrazol-4-yl)pyridin-2-yl)-2-(2-fluoro-6-methoxyphenyl)pyrimidin-4-amine hydrochloride

The titled compound (2.1 mg) was prepared in accordance with the same procedures as in Example 52, using Intermediates A3-2 and C1-1. (Yield: 24.7%) 1H NMR (CD₃OD, 400 MHz) δ 8.69 (d, 1H), 8.50 (s, 1H), 8.24 (s, 1H), 8.10 (s, 1H), 7.62-7.59 (m, 2H), 7.19 (s, 1H), 7.10 (d, 1H), 6.97 (t, 1H), 3.92 (s, 3H), 3.71 (d, 1H), 3.49-3.39 (m, 2H), 2.91-2.86 (m, 2H), 2.20-2.17 (m, 1H), 1.86-1.70 (m, 2H), 1.63-1.55 (m, 1H) MS m/z=511.1 (M+H)+

### Example 74: (S)-N-(4-(3-aminopiperidin-1-yl)-5-(1-(1-methylpiperidin-4-yl)-1H-pyrazol-4-yl)pyridin-2-yl)-2-(2-fluoro-6-methoxyphenyl)pyrimidin-4-amine hydrochloride

The titled compound (4.4 mg) was prepared in accordance with the same procedures as in Example 52, using Intermediates A3-3 and C1-1. (Yield: 47.9%) 1H NMR (CD₃OD, 400 MHz) δ 8.68 (d, 1H), 8.20-8.13 (m, 2H), 7.89 (s, 1H), 7.63-7.57 (m, 1H), 7.10-7.08 (m, 1H), 6.96 (t, 1H), 6.99-6.88 (m, 2H), 4.12-4.07 (m, 1H), 3.90 (s, 3H), 3.72-3.69 (m, 2H), 3.60-3.44 (m, 3H), 2.96 (s, 3H), 2.91-2.82 (m, 2H), 2.44-2.36 (m, 4H), 2.21-2.17 (m, 2H), 1.81-1.66 (m, 3H), 1.60-1.54 (m, 1H) MS m/z=558.3 (M+H)+

### Example 75: (3S)-1-(2-((5-chloro-6-(2-fluoro-6-methoxyphenyl)pyridin-2-yl)amino)-5-(1-(tetrahydro-2H-pyran-4-yl)-1H-pyrazol-4-yl)pyridin-4-yl)piperidin-3-ol

The titled compound (8.4 mg) was prepared in accordance with the same procedures as in Example 7, using Intermediates A2-1 and C1-3. (Yield: 24.4%) 1H NMR (CDCl₃, 400 MHz) δ 8.06 (s, 1H), 7.75 (s, 2H), 7.69 (d, 1H), 7.53-7.51 (m, 1H), 7.40-7.37 (m, 1H), 7.21 (s, 1H), 6.99-6.88 (m, 2H), 4.43-4.37 (m, 1H), 3.90 (d, 2H), 3.82-3.80 (m, 4H), 3.58 (t, 2H), 3.17-3.14 (m, 1H), 2.94-2.90 (m, 1H), 2.70-2.64 (m, 1H), 2.18-2.02 (m, 5H), 1.90-1.86 (m, 1H), 1.77-1.72 (m, 1H), 1.50-1.48 (m, 2H) MS m/z=579.2 (M+H)+

### Example 76: 4-((S)-3-aminopiperidin-1-yl)-5-(1-(difluoromethyl)-1H-pyrazol-4-yl)-N-(5-fluoro-6-(2-fluoro-6-methoxyphenyl)pyridin-2-yl)pyridin-2-amine hydrochloride

The titled compound (6.7 mg) was prepared in accordance with the same procedures as in Example 52, using Intermediates A3-2 and C1-2. (Yield: 53.9%) 1H NMR (CD₃OD, 400 MHz) δ 8.46 (s, 1H), 8.03 (d, 2H), 7.87 (t, 1H), 7.58 (t, 1H), 7.57-7.51 (m, 1H), 7.29 (d, 1H), 7.05 (d, 1H), 6.94-6.88 (m, 2H), 3.87 (s, 3H), 3.76 (d, 1H), 3.50-3.47 (m, 1H), 3.41-3.39 (m, 1H), 2.97-2.85 (m, 2H), 2.21-2.18 (m, 1H), 1.85-1.82 (m, 1H), 1.74-1.71 (m, 1H), 1.64-1.58 (m, 1H) MS m/z=528.2 (M+H)+

### Example 77: (R)-N-(4-(3-aminopiperidin-1-yl)-5-(1-(2,2,2-trifluoroethyl)-1H-pyrazol-4-yl)pyridin-2-yl)-2-(2-fluoro-6-methoxyphenyl)pyrimidin-4-amine hydrochloride

The titled compound (3.0 mg) was prepared in accordance with the same procedures as in Example 52, using Intermediates B3-34 and C1-1. (Yield: 27.0%) 1H NMR (CD₃OD, 400 MHz) δ 8.69 (d, 1H), 8.20 (d, 2H), 7.95 (s, 1H), 7.62-7.58 (m, 1H), 7.15 (s, 1H), 7.19 (d, 1H), 6.97 (t, 1H), 5.09-5.03 (m, 2H), 3.91 (s, 3H), 3.76-3.73 (m, 1H), 3.48-3.38 (m, 2H), 2.91-2.83 (m, 2H), 2.20-2.17 (m, 1H), 1.84-1.73 (m, 2H), 1.60-1.58 (m, 11H) MS m/z=543.2 (M+H)+

### Example 78: (S)-N-(4-(3-aminopiperidin-1-yl)-5-(1-(2,2,2-trifluoroethyl)-1H-pyrazol-4-yl)pyridin-2-yl)-2-(2-fluoro-6-methoxyphenyl)pyrimidin-4-amine hydrochloride

The titled compound (6.4 mg) was prepared in accordance with the same procedures as in Example 52, using Intermediates A3-4 and C1-1. (Yield: 57.6%) 1H NMR (CD₃OD, 400 MHz) δ 8.69 (d, 1H), 8.20 (d, 2H), 7.95 (s, 1H), 7.62-7.60 (m, 1H), 7.15 (s, 1H), 7.19 (d, 1H), 6.97 (t, 1H), 5.09-5.02 (m, 2H), 3.91 (s, 3H), 3.75-3.73 (m, 1H), 3.48-3.36 (m, 2H), 2.91-2.83 (m, 2H), 2.20-2.17 (m, 1H), 1.81-1.70 (m, 2H), 1.62-1.55 (m, 1H) MS m/z=543.2 (M+H)+

### Example 79: (S)-N-(4-(3-aminopiperidin-1-yl)-5-((tetrahydro-2H-pyran-4-yl)ethynyl)pyridin-2-yl)-2-(2-fluoro-6-methoxyphenyl)pyrimidin-4-amine hydrochloride

The titled compound (8.2 mg) was prepared in accordance with the same procedures as in Example 52, using Intermediates A7-1 and C1-1. (Yield: 16.3%) 1H NMR (CD₃OD, 400 MHz) δ 8.73 (d, 1H), 8.28 (s, 1H), 7.68-7.64 (m, 2H), 7.14-7.11 (m, 2H), 7.03 (dd, 1H), 4.31-4.29 (m, 2H), 3.95 (s, 3H), 3.91-3.90 (m, 2H), 3.58-3.50 (m, 1H), 3.48-3.47 (m, 1H), 3.35-3.31 (m, 1H), 3.00-2.99 (m, 1H), 1.97-1.94 (m, 1H), 1.80-1.74 (m, 3H), 1.20-1.18 (m, 4H) MS m/z=503.0 (M+H)+

### Example 80: (S)-3-(4-((4-(3-aminopiperidin-1-yl)-5-(1-(tetrahydro-2H-pyran-4-yl)-1H-pyrazol-4-yl)pyridin-2-yl)amino)pyrimidin-2-yl)pyridin-2-ol hydrochloride

The titled compound (11.5 mg) was prepared in accordance with the same procedures as in Example 52, using Intermediates A3-1 and C1-9. (Yield: 84.9%) 1H NMR (CD₃OD, 400 MHz) δ 9.00 (d, 1H), 8.67 (d, 1H), 8.27 (s, 1H), 8.20 (s 1H), 7.93 (d, 1H), 7.89 (s, 1H), 7.50-7.20 (m, 1H), 7.13 (s, 1H), 6.76 (t, 1H), 4.57-4.54 (m, 1H), 4.11 (d, 2H), 3.62 (t, 2H), 3.49-3.31 (m, 1H), 2.25-2.18 (m, 2H), 2.18-2.14 (m, 4H), 1.90-1.57 (m, 4H), 0.92-0.89 (m, 2H) MS m/z=514.2 (M+H)+

### Example 81: (S)-1-(6′-((2-(2-fluoro-6-methoxyphenyl)pyrimidin-4-yl)amino)-5-((1-methylpiperidin-4-yl)oxy)-[2,3′-bipyridin]-4′-yl)piperidin-3-ol

The titled compound (8.5 mg) was prepared in accordance with the same procedures as in Example 7, using Intermediates A4-9 and C1-1. (Yield: 30.6%) 1H NMR (CD₃OD, 400 MHz) δ 8.43 (d, 1H), 8.34 (s, 1H), 8.12 (s 1H), 7.76 (d, 1H), 7.66 (s, 1H), 7.56-7.51 (m, 2H), 7.45 (q, 1H), 6.97 (d, 1H), 6.85 (t, 1H), 4.75-4.60 (m, 1H), 3.83 (s, 3H), 3.68-3.48 (m, 1H), 3.10-2.95 (m, 2H), 2.88-2.70 (m, 2H), 2.57 (s, 3H), 2.20-2.05 (m, 2H), 2.05-1.85 (m, 4H), 1.64-1.38 (m, 4H), 0.98-0.80 (m, 2H) MS m/z=586.3 (M+H)+

### Example 82: (S)-4-(3-aminopiperidin-1-yl)-N-(2-(2-fluoro-6-methoxyphenyl)pyrimidin-4-yl)-6′-(tetrahydro-2H-pyran-4-yl)-[3,3′-bipyridin]-6-amine hydrochloride

The titled compound (9.0 mg) was prepared in accordance with the same procedures as in Example 52, using Intermediates A5-1 and C1-1. (Yield: 44.7%) 1H NMR (CD₃OD, 400 MHz) δ 9.03-9.01 (m, 1H), 8.86-8.84 (m, 1H), 8.68-8.66 (m, 1H), 8.32 (s, 1H), 8.12-8.10 (m, 1H), 7.70-7.64 (m, 1H), 7.14-7.12 (m, 1H), 7.03 (d, 1H), 6.99 (dd, 1H), 4.15-4.12 (m, 2H), 3.94 (s, 3H), 3.67-3.61 (m, 3H), 3.48-3.40 (m, 2H), 2.83-2.77 (m, 2H), 2.15-1.99 (m, 5H), 1.82-1.79 (m, 1H), 1.66-1.64 (m, 1H), 1.55-1.53 (m, 1H) MS m/z=556.2 (M+H)+

### Example 83: (S)-4-(3-aminopiperidin-1-yl)-N-(2-(2-fluoro-6-methoxyphenyl)pyrimidin-4-yl)-6′-(1-methylpiperidin-4-yl)-[3,3′-bipyridin]-6-amine hydrochloride

The titled compound (6.3 mg) was prepared in accordance with the same procedures as in Example 52, using Intermediates A5-2 and C1-1. (Yield: 30.6%) 1H NMR (CD₃OD, 400 MHz) δ 9.07 (s, 1H), 8.81 (s, 1H), 8.76-8.74 (m, 1H), 8.34 (s, 1H), 8.16-8.14 (m, 1H), 7.72-7.67 (m, 1H), 7.44 (s, 1H), 7.16 (d, 1H), 7.05 (dd, 1H), 3.96 (s, 3H), 3.75-3.73 (m, 2H), 3.66-3.45 (m, 2H), 3.1 (s, 3H), 2.97-2.92 (m, 1H), 2.49-2.47 (m, 2H), 2.46-2.43 (m, 2H), 2.33-2.30 (m, 1H), 2.26-2.17 (m, 1H), 2.14-2.04 (m, 2H), 2.02-1.83 (m, 1H), 1.72-1.58 (m, 2H), 1.56-1.40 (m, 2H) MS m/z=569.3 (M+H)+

### Example 84: 2-(2-fluoro-6-methoxyphenyl)-N-(5-(1-(tetrahydro-2H-pyran-4-yl)-1H-pyrazol-4-yl)-4-(3-(2,2,2-trifluoroethyl)piperidin-1-yl)pyridin-2-yl)pyrimidin-4-amine

The titled compound (23.2 mg) was prepared in accordance with the same procedures as in Example 7, using Intermediates B3-13 and C1-1. (Yield: 67.5%) 1H NMR (CD₃OD, 400 MHz) δ 8.42 (d, 1H), 8.08 (s, 1H), 8.03 (s, 1H), 7.82 (s, 1H), 7.71 (s, 1H), 7.47-7.41 (m, 2H), 6.97 (d, 1H), 6.84 (t, 1H), 4.49-4.43 (m, 1H), 4.09 (d, 2H), 3.82 (s, 3H), 3.63-3.56 (m, 3H), 3.32 (d, 1H), 3.22 (d, 1H), 2.19 (t, 1H), 2.15-2.09 (m, 5H), 2.09-1.83 (m, 3H), 1.72-1.60 (m, 2H), 1.16-1.12 (m, 1H) MS m/z=612.2 (M+H)+

### Example 85: 2-(2-fluoro-6-methoxyphenyl)-N-(4-(3-(fluoromethyl)piperidin-1-yl)-5-(1-(tetrahydro-2H-pyran-4-yl)-1H-pyrazol-4-yl)pyridin-2-yl)pyrimidin-4-amine

The titled compound (20.5 mg) was prepared in accordance with the same procedures as in Example 7, using Intermediates B3-14 and C1-1. (Yield: 66.1%) 1H NMR (CD₃OD, 400 MHz) δ 8.41 (d, 1H), 8.08 (s, 1H), 8.03 (s, 1H), 7.82 (s, 1H), 7.71 (s, 1H), 7.47-7.41 (m, 2H), 6.96 (d, 1H), 6.85 (t, 1H), 4.50-4.45 (m, 1H), 4.09 (d, 2H), 3.82 (s, 3H), 3.63-3.56 (m, 3H), 3.33 (d, 1H), 3.23 (d, 1H), 2.50 (t, 1H), 2.33 (t, 1H), 2.15-1.95 (m, 6H), 1.76 (d, 1H), 1.76-1.60 (m, 2H), 1.20-1.12 (m, 1H) MS m/z=562.3 (M+H)+

### Example 86: N-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-yl)acetamide

The titled compound (11.0 mg) was prepared in accordance with the same procedures as in Example 7, using Intermediates B3-15 and C1-1. (Yield: 38.0%) 1H NMR (CD₃OD, 400 MHz) δ 8.41 (d, 1H), 8.10 (s, 1H), 8.08 (s, 1H), 7.81 (s, 1H), 7.64 (s, 1H), 7.50-7.41 (m, 2H), 6.96 (d, 1H), 6.85 (t, 1H), 4.59-4.44 (m, 1H), 4.09 (d, 2H), 3.87 (t, 1H), 3.82 (s, 3H), 3.60 (t, 2H), 3.14 (d, 1H), 2.56 (t, 1H), 2.41 (t, 1H), 2.16-2.05 (m, 4H), 1.95-1.82 (m, 1H), 1.91 (s, 3H), 1.67-1.61 (m, 2H), 1.38-1.33 (m, 2H) MS m/z=587.3 (M+H)+

### Example 87: 2-(2-fluoro-6-methoxyphenyl)-N-(5-(1-(tetrahydro-2H-pyran-4-yl)-1H-pyrazol-4-yl)-4-(4-(trifluoromethyl)piperidin-1-yl)pyridin-2-yl)pyrimidin-4-amine

The titled compound (23.7 mg) was prepared in accordance with the same procedures as in Example 7, using Intermediates B3-16 and C1-1. (Yield: 69.0%) 1H NMR (CD₃OD, 400 MHz) δ 8.42 (d, 1H), 8.10 (s, 1H), 8.05 (s, 1H), 7.91 (s, 1H), 7.84 (s, 1H), 7.70-7.42 (m, 2H), 6.97 (d, 1H), 6.85 (t, 1H), 4.58-4.44 (m, 1H), 4.09 (d, 2H), 3.82 (s, 3H), 3.79-3.57 (m, 3H), 3.41 (d, 1H), 2.58 (t, 2H), 2.35-2.20 (m, 1H), 2.20-1.98 (m, 4H), 1.87-1.76 (m, 2H), 1.65-1.56 (m, 2H) MS m/z=598.2 (M+H)+

### Example 88: (S)-N-(4-(3-aminopiperidin-1-yl)-5-(1-(difluoromethyl)-1H-pyrazol-4-yl)pyridin-2-yl)-2-(7-fluoroquinoxalin-6-yl)pyrimidin-4-amine hydrochloride

The titled compound (9.9 mg) was prepared in accordance with the same procedures as in Example 52, using Intermediates A3-2 and C1-6. (Yield: 46.6%) 1H NMR (CD₃OD, 400 MHz) δ 9.12 (d, 1H), 9 (d, 2H), 8.9 (d, 1H), 8.56 (s, 1H), 8.27 (s, 1H), 8.12 (s, 1H), 8.06 (d, 1H), 7.77-7.47 (m, 1H), 7.28 (s, 1H), 7.06 (s, 1H), 3.85-3.83 (m, 1H), 3.60-3.57 (m, 1H), 3.48-3.43 (m, 1H), 3.03-2.94 (m, 2H), 2.22-2.20 (m, 1H), 1.88-1.85 (m, 1H), 1.77-1.74 (m, 1H), 1.64-1.61 (m, 1H) MS m/z=533.1 (M+H)+

### Example 89: (S)-N-(4-(3-aminopiperidin-1-yl)-5-(1-(2,2,2-trifluoroethyl)-1H-pyrazol-4-yl)pyridin-2-yl)-2-(7-fluoroquinoxalin-6-yl)pyrimidin-4-amine hydrochloride

The titled compound (1.4 mg) was prepared in accordance with the same procedures as in Example 52, using Intermediates A3-4 and C1-6. (Yield: 6.3%) 1H NMR (CD₃OD, 400 MHz) δ 9.11 (d, 1H), 9 (d, 2H), 8.9 (d, 1H), 8.25 (s, 1H), 8.2 (s, 1H), 8.06 (d, 1H), 7.97 (s, 1H), 7.26 (d, 1H), 7.05 (s, 1H), 5.10-5.08 (m, 2H), 3.89-3.86 (m, 1H), 3.60-3.57 (m, 1H), 3.48-3.45 (m, 1H), 3.04-2.90 (m, 2H), 2.22-2.20 (m, 1H), 1.76-1.73 (m, 1H), 1.66-1.63 (m, 1H), 1.40-1.30 (m, 1H) MS m/z=565.1 (M+H)+

### Example 90: (S)-N-(4-(3-aminopiperidin-1-yl)-5-((tetrahydro-2H-pyran-4-yl)ethynyl)pyridin-2-yl)-2-(7-fluoroquinoxalin-6-yl)pyrimidin-4-amine hydrochloride

The titled compound (7.2 mg) was prepared in accordance with the same procedures as in Example 52, using Intermediates A7-1 and C1-6. (Yield: 31.5%) 1H NMR (CD₃OD, 400 MHz) δ 9.08 (d, 1H), 9 (d, 2H), 8.86 (d, 1H), 8.33 (s, 1H), 8.07 (d, 1H), 7.26-7.25 (m, 1H), 6.93 (s, 1H), 4.33-4.26 (m, 2H), 3.96-3.93 (m, 2H), 3.59-3.49 (m, 3H), 3.40-3.35 (m, 2H), 3.03-3.01 (m, 1H), 2.27-2.25 (m, 1H), 2.01-1.95 (m, 2H), 1.89-1.81 (m, 2H), 1.79-1.77 (m, 4H) MS m/z=578.2 (M+H)+

### Example 91: (S)-N-(4-(3-aminopiperidin-1-yl)-5-(1-(2-methoxyethyl)-1H-pyrazol-4-yl)pyridin-2-yl)-2-(2-fluoro-6-methoxyphenyl)pyrimidin-4-amine hydrochloride

The titled compound (7.7 mg) was prepared in accordance with the same procedures as in Example 52, using Intermediates A3-5 and C1-1. (Yield: 34.3%) 1H NMR (CD₃OD, 400 MHz) δ 8.74-8.70 (m, 1H), 8.14 (s, 1H), 8.06 (s, 1H), 7.83 (s, 1H), 7.62-7.50 (m, 1H), 7.17-7.10 (m, 2H), 6.98-6.96 (m, 1H), 4.40-4.39 (m, 2H), 3.92-3.91 (m, 3H), 3.84-3.81 (m, 3H), 3.53-3.49 (m, 1H), 3.42-3.37 (m, 4H), 2.90-2.85 (m, 2H), 2.21-2.18 (m, 1H), 1.88-1.79 (m, 2H), 1.61-1.60 (m, 1H) MS m/z=519.3 (M+H)+

### Example 92: N-(4-(2,8-diazaspiro[4.5]decan-8-yl)-5-(1-(tetrahydro-2H-pyran-4-yl)-1H-pyrazol-4-yl)pyridin-2-yl)-2-(2-fluoro-6-methoxyphenyl)pyrimidin-4-amine hydrochloride

The titled compound (29.0 mg) was prepared in accordance with the same procedures as in Example 52, using Intermediates B3-17 and C1-1. (Yield: 84.8%) 1H NMR (CD₃OD, 400 MHz) δ 8.75 (d, 1H), 8.09 (s, 1H), 8.04 (s, 1H), 7.80 (s, 1H), 7.62 (q, 1H), 7.36 (s, 1H), 7.15-7.10 (m, 2H), 6.98 (t, 1H), 4.60-4.45 (m, 1H), 4.09 (d, 2H), 3.92 (s, 3H), 3.60 (t, 2H), 3.43-3.31 (m, 6H), 3.19 (s, 2H), 2.18-2.05 (m, 4H), 2.02 (t, 2H), 1.85-1.65 (m, 4H) MS m/z=585.3 (M+H)+

### Example 93: N-(4-(2,8-diazaspiro[4.5]decan-2-yl)-5-(1-(tetrahydro-2H-pyran-4-yl)-1H-pyrazol-4-yl)pyridin-2-yl)-2-(2-fluoro-6-methoxyphenyl)pyrimidin-4-amine hydrochloride

The titled compound (36.3 mg) was prepared in accordance with the same procedures as in Example 52, using Intermediates B3-18 and C1-1. (Yield: 98.2%) 1H NMR (CD₃OD, 400 MHz) δ 8.74 (d, 1H), 7.94 (s, 1H), 7.85 (s, 1H), 7.66-7.60 (m, 1H), 7.31 (d, 1H), 7.11 (d, 1H), 6.98 (t, 1H), 6.79 (s, 1H), 4.52-4.47 (m, 1H), 4.08 (d, 2H), 3.92 (s, 3H), 3.60 (t, 2H), 3.57-3.31 (m, 2H), 3.31-3.16 (m, 6H), 2.20-2.02 (m, 4H), 1.96 (t, 2H), 1.86-1.70 (m, 4H) MS m/z=585.3 (M+H)+

### Example 94: (3R,4R)-4-fluoro-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-ol

The titled compound (20.5 mg) was prepared in accordance with the same procedures as in Example 7, using Intermediates B3-19 and C1-1. (Yield: 39.8%) 1H NMR (CD₃OD, 400 MHz) δ 8.42 (d, 1H), 8.11 (s, 1H), 8.10 (s, 1H), 7.84 (s, 1H), 7.68 (s, 1H), 7.50-7.41 (m, 2H), 6.96 (d, 1H), 6.85 (t, 1H), 4.50-4.48 (m, 1H), 4.48-4.20 (m, 1H), 4.10 (d, 2H), 3.83 (s, 3H), 3.80-3.65 (m, 1H), 3.60 (t, 2H), 3.35-3.17 (m, 2H), 2.72 (t, 1H), 2.52 (t, 1H), 2.15-2.05 (m, 4H), 2.05-1.96 (m, 1H), 1.30-1.22 (m, 1H) MS m/z=564.2 (M+H)+

### Example 95: (3R,4R)-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)-4-(hydroxymethyl)piperidin-3-ol

The titled compound (11.4 mg) was prepared in accordance with the same procedures as in Example 7, using Intermediates B3-20 and C1-1. (Yield: 26.7%) 1H NMR (CD₃OD, 400 MHz) δ 8.42 (d, 1H), 8.11 (s, 1H), 8.05 (s, 1H), 7.83 (s, 1H), 7.61 (s, 1H), 7.52 (d, 1H), 7.44 (q, 1H), 6.96 (d, 1H), 6.85 (t, 1H), 4.52-4.40 (m, 1H), 4.09 (d, 2H), 3.83 (s, 3H), 3.83-3.74 (m, 1H), 3.68-3.55 (m, 3H), 3.55-3.45 (m, 1H), 3.45-3.36 (m, 1H), 3.36 (s, 2H), 2.53 (t, 1H), 2.29 (t, 1H), 2.20-2.04 (m, 4H), 1.80-1.68 (m, 1H), 1.50-1.34 (m, 1H) MS m/z=576.2 (M+H)+

### Example 96: (R)-N-(4-(3-aminopyrrolidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine hydrochloride

The titled compound (23.2 mg) was prepared in accordance with the same procedures as in Example 52, using Intermediates B3-21 and C1-1. (Yield: 80.8%) 1H NMR (CD₃OD, 400 MHz) δ 8.74 (d, 1H), 7.96 (s, 1H), 7.89 (s, 1H), 7.65 (s, 1H), 7.60 (q, 1H), 7.26 (d, 1H), 7.09 (d, 1H), 6.95 (t, 1H), 6.73 (s, 1H), 4.53-4.44 (m, 1H), 4.08 (d, 2H), 4.04-3.94 (m, 1H), 3.91 (s, 3H), 3.75-3.50 (m, 5H), 3.45-3.35 (m, 1H), 2.48-2.32 (m, 1H), 2.20-2.00 (m, 5H) MS m/z=531.2 (M+H)+

### Example 97: (S)-N-(4-(3-aminopyrrolidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine hydrochloride

The titled compound (23.7 mg) was prepared in accordance with the same procedures as in Example 52, using Intermediates B3-22 and C1-1. (Yield: 79.6%) 1H NMR (CD₃OD, 400 MHz) δ 8.74 (d, 1H), 7.97 (s, 1H), 7.89 (s, 1H), 7.65 (s, 1H), 7.61 (q, 1H), 7.29 (d, 1H), 7.09 (d, 1H), 6.96 (t, 1H), 6.76 (s, 1H), 4.53-4.44 (m, 1H), 4.08 (d, 2H), 4.00-3.92 (m, 1H), 3.92 (s, 3H), 3.73-3.60 (m, 2H), 3.60-3.56 (m, 3H), 3.50-3.35 (m, 1H), 2.43-2.37 (m, 1H), 2.20-2.00 (m, 5H) MS m/z=531.2 (M+H)+

### Example 98: (S)-N-(4-(3-aminopiperidin-1-yl)-5-(2-(1-methylpiperidin-4-yl)thiazol-5-yl)pyridin-2-yl)-2-(2-fluoro-6-methoxyphenyl)pyrimidin-4-amine hydrochloride

The titled compound (10.2 mg) was prepared in accordance with the same procedures as in Example 52, using Intermediates A5-3 and C1-1. (Yield: 96.6%) 1H NMR (CD₃OD, 400 MHz) δ 8.44 (d, 1H), 8.18 (s, 1H), 7.75 (s, 1H), 7.55 (s, 1H), 7.45 (q, 1H), 6.97 (d, 1H), 6.87 (t, 1H), 3.82 (s, 3H), 3.64 (brs, 1H), 3.12-3.05 (m, 5H), 2.56 (brs, 1H), 2.40 (s, 3H), 2.38 (t, 3H), 2.22 (d, 2H), 1.98-1.88 (m, 4H), 1.66 (s, 2H), 1.42 (s, 9H) MS m/z=575.2 (M+H)+

### Example 99: (S)-4-(3-aminopiperidin-1-yl)-N-(2-(7-fluoroquinoxalin-6-yl)pyrimidin-4-yl)-6′-(tetrahydro-2H-pyran-4-yl)-[3,3′-bipyridin]-6-amine hydrochloride

The titled compound (7.2 mg) was prepared in accordance with the same procedures as in Example 52, using Intermediates A5-1 and C1-6. (Yield: 31.5%) 1H NMR (CD₃OD, 400 MHz) δ 9.10-9.07 (m, 2H), 9 (s, 2H), 8.98 (d, 1H), 8.89 (d, 1H), 8.82 (s, 1H), 8.37 (d, 1H), 8.15 (d, 1H), 7.39 (s, 1H), 7.2 (s, 1H), 4.15-4.13 (m, 2H), 3.86-3.83 (m, 1H), 3.68-3.62 (m, 2H), 3.51-3.41 (m, 3H), 3.07-3.01 (m, 2H), 2.19-2.17 (m, 1H), 2.06-2.01 (m, 4H), 1.85-1.82 (m, 1H), 1.70-1.59 (m, 2H) MS m/z=578.2 (M+H)+

### Example 100: 4-((S)-3-aminopiperidin-1-yl)-N-(5-fluoro-6-(2-fluoro-6-methoxyphenyl)pyridin-2-yl)-5-(1-(2,2,2-trifluoroethyl)-1H-pyrazol-4-yl)pyridin-2-amine hydrochloride

The titled compound (6.5 mg) was prepared in accordance with the same procedures as in Example 52, using Intermediates A3-4 and C1-2. (Yield: 60.4%) 1H NMR (CD₃OD, 400 MHz) δ 8.17 (s, 1H), 7.98 (s, 1H), 7.89-7.86 (m, 2H), 7.54-7.52 (m, 1H), 7.30 (d, 1H), 7.06-7.03 (m, 1H), 6.93-6.91 (m, 1H), 6.90-6.88 (m, 2H), 5.06 (q, 2H), 3.87 (s, 3H), 3.80-3.78 (m, 1H), 3.47-3.41 (m, 2H), 2.94-2.91 (m, 1H), 2.87-2.81 (m, 1H), 2.20-2.17 (m, 1H), 1.73-1.70 (m, 1H), 1.66-1.64 (m, 1H), 1.61-1.58 (m, 1H) MS m/z=560.2 (M+H)+

### Example 101: (S)-(4-(4-((4-(3-aminopiperidin-1-yl)-5-(1-(difluoromethyl)-1H-pyrazol-4-yl)pyridin-2-yl)amino)pyrimidin-2-yl)-3-fluoro-5-methoxyphenyl)methanol hydrochloride

The titled compound (5.9 mg) was prepared in accordance with the same procedures as in Example 52, using Intermediates A3-2 and C5-1. (Yield: 80.2%) 1H NMR (CD₃OD, 400 MHz) δ 8.71 (d, 1H), 8.52 (s, 1H), 8.23 (s, 1H), 8.10 (s, 1H), 7.75-7.45 (m, 1H), 7.25 (s, 1H), 7.10 (s, 1H), 7.00 (d, 1H), 4.72 (s, 2H), 3.94 (s, 3H), 3.77-3.74 (m, 1H), 3.48-3.39 (m, 2H), 2.97-2.87 (m, 2H), 2.20-2.17 (m, 1H), 1.86-1.82 (m, 1H), 1.75-1.72 (m, 1H), 1.62-1.59 (m, 1H) MS m/z=541.2 (M+H)+

### Example 102: (S)-(4-(4-((4-(3-aminopiperidin-1-yl)-5-(1-(2,2,2-trifluoroethyl)-1H-pyrazol-4-yl)pyridin-2-yl)amino)pyrimidin-2-yl)-3-fluoro-5-methoxyphenyl)methanol hydrochloride

The titled compound (2.2 mg) was prepared in accordance with the same procedures as in Example 52, using Intermediates A3-4 and C5-1. (Yield: 28.4%) 1H NMR (CD₃OD, 400 MHz) δ 8.69 (d, 1H), 8.22-8.19 (m, 2H), 7.95 (s, 1H), 7.18 (s, 1H), 7.08 (s, 1H), 6.98 (d, 1H), 5.07 (q, 2H), 4.72 (s, 2H), 3.92 (s, 3H), 3.76-3.74 (m, 1H), 3.47-3.39 (m, 2H), 2.92-2.85 (m, 2H), 2.19-2.17 (m, 1H), 1.75-1.72 (m, 2H), 1.60-1.58 (m, 1H) MS m/z=573.2 (M+H)+

### Example 103: (S)-(4-(4-((4-(3-aminopiperidin-1-yl)-5-((tetrahydro-2H-pyran-4-yl)ethynyl)pyridin-2-yl)amino)pyrimidin-2-yl)-3-fluoro-5-methoxyphenyl)methanol hydrochloride

The titled compound (2.7 mg) was prepared in accordance with the same procedures as in Example 52, using Intermediates A7-1 and C5-1. (Yield: 37.2%) 1H NMR (CD₃OD, 400 MHz) δ 8.64 (d, 1H), 8.26 (s, 1H), 7.09 (s, 2H), 6.99 (d, 1H), 4.72 (s, 2H), 4.17-4.15 (m, 1H), 4.01-4.00 (m, 1H), 3.94 (s, 3H), 3.93-3.91 (m, 2H), 3.58-3.53 (m, 2H), 3.48-3.46 (m, 1H), 3.23-3.20 (m, 2H), 2.99-2.98 (m, 1H), 2.24-2.21 (m, 1H), 1.97-1.94 (m, 3H), 1.77-1.75 (m, 4H) MS m/z=533.3 (M+H)+

### Example 104: (S)-N-(4-(3-aminopiperidin-1-yl)-5-(1-(difluoromethyl)-1H-pyrazol-4-yl)pyridin-2-yl)-2-(4-((dimethylamino)methyl)-2-fluoro-6-methoxyphenyl)pyrimidin-4-amine hydrochloride

The titled compound (10.7 mg) was prepared in accordance with the same procedures as in Example 52, using Intermediates A3-2 and C6-1. (Yield: 55.7%) 1H NMR (CDCl₃, 400 MHz) δ 8.8 (d, 1H), 8.54 (s, 1H), 8.25 (s, 1H), 8.08 (s, 1H), 7.61 (s, 1H), 7.51 (s, 1H), 7.24-7.23 (m, 2H), 4.47 (s, 2H), 4.01 (s, 3H), 3.89-3.87 (m, 1H), 3.58-3.56 (m, 1H), 3.44-3.42 (m, 1H), 3.12-3.07 (m, 2H), 2.96 (s, 6H), 2.22-2.19 (m, 1H), 1.83-1.81 (m, 1H), 1.69-1.67 (m, 2H) MS m/z=568.2 (M+H)+

### Example 105: (S)-N-(4-(3-aminopiperidin-1-yl)-5-(1-(2,2,2-trifluoroethyl)-1H-pyrazol-4-yl)pyridin-2-yl)-2-(4-((dimethylamino)methyl)-2-fluoro-6-methoxyphenyl)pyrimidin-4-amine hydrochloride

The titled compound (15.1 mg) was prepared in accordance with the same procedures as in Example 52, using Intermediates A3-4 and C6-1. (Yield: 74.9%) 1H NMR (CD₃OD, 400 MHz) δ 8.81-8.80 (m, 1H), 8.26 (s, 1H), 8.2 (s, 1H), 7.93 (s, 1H), 7.49-7.48 (m, 1H), 7.41 (s, 1H), 7.23-7.22 (m, 2H), 5.09 (q, 2H), 4.47 (s, 2H), 4 (s, 3H), 3.92-3.89 (m, 1H), 3.60-3.57 (m, 1H), 3.48-3.46 (m, 1H), 3.35-3.31 (m, 1H), 3.07 (s, 6H), 2.21-2.19 (m, 1H), 1.80-1.69 (m, 3H) MS m/z=600.3 (M+H)+

### Example 106: (S)-N-(4-(3-aminopiperidin-1-yl)-5-((tetrahydro-2H-pyran-4-yl)ethynyl)pyridin-2-yl)-2-(4-((dimethylamino)methyl)-2-fluoro-6-methoxyphenyl)pyrimidin-4-amine hydrochloride

The titled compound (9.0 mg) was prepared in accordance with the same procedures as in Example 52, using Intermediates A7-1 and C6-1. (Yield: 47.5%) 1H NMR (CD₃OD, 400 MHz) δ 8.74-8.72 (m, 1H), 8.27 (s, 1H), 7.47 (s, 1H), 7.36 (s, 1H), 7.2 (d, 1H), 6.98 (s, 1H), 4.45 (s, 2H), 4.30-4.27 (m, 2H), 3.97 (s, 3H), 3.93-3.90 (m, 2H), 3.57-3.48 (m, 3H), 3.12-3.00 (m, 1H), 2.96 (s, 6H), 2.26-2.23 (m, 1H), 2.04-1.96 (m, 3H), 1.93-1.74 (m, 4H) MS m/z=560.3 (M+H)+

### Example 107: (S)-N-(4-(3-aminopiperidin-1-yl)-5-(1-(trifluoromethyl)-1H-pyrazol-4-yl)pyridin-2-yl)-2-(2-fluoro-6-methoxyphenyl)pyrimidin-4-amine hydrochloride

The titled compound (30.8 mg) was prepared in accordance with the same procedures as in Example 52, using Intermediates A3-11 and C1-1. (Yield: 66.8%) 1H NMR (CD₃OD, 400 MHz) δ 8.83 (d, 1H), 8.68 (s, 1H), 8.25 (d, 2H), 7.74-7.69 (m, 1H), 7.63-7.61 (m, 1H), 7.37 (s, 1H), 7.18 (d, 1H), 7.06 (dd, 1H), 3.99 (s, 3H), 3.94-3.91 (m, 1H), 3.62-3.59 (m, 1H), 3.48-3.44 (m, 1H), 3.16-3.10 (m, 1H), 3.07-3.02 (m, 1H), 2.22-2.20 (m, 1H), 1.74-1.71 (m, 1H), 1.68-1.66 (m, 2H) MS m/z=529.1 (M+H)+

### Example 108: (S)-N-(4-(3-aminopiperidin-1-yl)-5-(1-(difluoromethyl)-1H-pyrazol-4-yl)pyridin-2-yl)-2-(2,4-difluoro-6-methoxyphenyl)pyrimidin-4-amine hydrochloride

The titled compound (29.2 mg) was prepared in accordance with the same procedures as in Example 52, using Intermediates A3-2 and C1-7. (Yield: 89.5%) 1H NMR (CD₃OD, 400 MHz) δ 8.83-8.82 (d, 1H), 8.57 (s, 1H), 8.23 (s, 1H), 8.09 (s, 1H), 7.63-7.57 (m, 2H), 7.35 (s, 1H), 7.06 (d, 1H), 6.92 (dd, 1H), 3.99 (s, 3H), 3.96-3.93 (m, 1H), 3.64-3.61 (m, 1H), 3.48-3.46 (m, 1H), 3.17-3.16 (m, 1H), 3.14-3.04 (m, 1H), 2.22-2.20 (m, 1H), 1.83-1.69 (m, 3H) MS m/z=529.2 (M+H)+

### Example 109: (S)-N-(4-(3-aminopiperidin-1-yl)-5-(1-(2,2,2-trifluoroethyl)-1H-pyrazol-4-yl)pyridin-2-yl)-2-(2,4-difluoro-6-methoxyphenyl)pyrimidin-4-amine

The titled compound (56.6 mg) was prepared in accordance with the same procedures as in Example 52, using Intermediates A3-4 and C1-7. (Yield: 72.5%) 1H NMR (CD₃OD, 400 MHz) δ 8.43 (d, 1H), 8.11 (s, 2H), 7.92 (s, 1H), 7.57 (s, 2H), 6.83 (d, 1H), 6.7 (dd, 1H), 5.02 (q, 2H), 3.82 (s, 3H), 3.17-3.13 (m, 1H), 2.87-2.84 (m, 1H), 2.58-2.53 (m, 1H), 2.35-2.30 (m, 1H), 2.04-2.02 (m, 1H), 1.97-1.94 (m, 1H), 1.26-1.20 (m, 2H) MS m/z=560.9 (M+H)+

### Example 110: (S)-N-(4-(3-aminopiperidin-1-yl)-5-((tetrahydro-2H-pyran-4-yl)ethynyl)pyridin-2-yl)-2-(2,4-difluoro-6-methoxyphenyl)pyrimidin-4-amine

The titled compound (5.7 mg) was prepared in accordance with the same procedures as in Example 52, using Intermediates A7-1 and C1-7. (Yield: 40.0%) 1H NMR (CD₃OD, 400 MHz) δ 8.42 (d, 1H), 8.09 (s, 1H), 7.53-7.51 (m, 1H), 7.46 (s, 1H), 6.82 (d, 1H), 6.72 (dd, 1H), 3.95-3.92 (m, 2H), 3.89 (s, 3H), 3.72-3.69 (m, 1H), 3.58-3.55 (m, 2H), 2.95-2.83 (m, 3H), 2.64-2.59 (m, 1H), 2.21-2.18 (m, 2H), 2.04-1.93 (m, 2H), 1.80-1.70 (m, 3H), 1.66-1.61 (m, 1H), 1.25-1.23 (m, 1H)

### Example 111: (S)-N-(4-(3-aminopiperidin-1-yl)-5-((1-(trifluoromethyl)-1H-pyrazol-4-yl)ethynyl)pyridin-2-yl)-2-(2-fluoro-6-methoxyphenyl)pyrimidin-4-amine hydrochloride

The titled compound (28.0 mg) was prepared in accordance with the same procedures as in Example 52, using Intermediates A7-2 and C1-1. (Yield: 84.5%) 1H NMR (CD₃OD, 400 MHz) δ 8.73-8.72 (m, 1H), 8.62-8.60 (m, 1H), 8.42 (s, 1H), 8.06 (s, 1H), 7.70-7.67 (m, 1H), 7.21 (s, 1H), 7.16-7.14 (m, 1H), 7.04-7.00 (m, 1H), 4.32-4.29 (m, 1H), 4.13-4.10 (m, 1H), 3.96 (s, 3H), 3.52-3.48 (m, 2H), 2.32-2.22 (m, 1H), 2.12-2.02 (m, 1H), 1.89-1.86 (m, 1H), 1.76-1.64 (m, 1H)

### Example 112: 2-(2-fluoro-6-methoxyphenyl)-6-((4-((S)-3-hydroxypiperidin-1-yl)-5-(1-(tetrahydro-2H-pyran-4-yl)-1H-pyrazol-4-yl)pyridin-2-yl)amino)nicotinamide

### Step 1: methyl 2-(2-fluoro-6-methoxyphenyl)-6-((4-((S)-3-hydroxypiperidin-1-yl)-5-(1-(tetrahydro-2H-pyran-4-yl)-1H-pyrazol-4-yl)pyridin-2-yl)amino)nicotinate

The titled compound (105.2 mg) was prepared in accordance with the same procedures as in Example 7, using Intermediates A2-1 and C2-1. (Yield: 48.2%) 1H NMR (CDCl₃, 400 MHz) δ 8.25 (d, 1H), 8.07 (s, 1H), 7.76 (s, 2H), 7.62 (s, 1H), 7.55 (s, 1H), 7.37-7.31 (m, 2H), 6.84-6.76 (m, 2H), 4.41-4.39 (m, 1H), 4.16-4.12 (m, 2H), 3.84-3.77 (m, 1H), 3.75 (s, 3H), 3.69 (s, 3H), 3.58 (t, 2H), 3.15-3.12 (m, 1H), 2.95-2.87 (m, 1H), 2.73-2.61 (m, 2H), 2.18-2.06 (m, 4H), 1.87-1.85 (m, 1H), 1.78-1.70 (m, 1H), 1.49-1.40 (m, 2H)

### Step 2: 2-(2-fluoro-6-methoxyphenyl)-6-((4-((S)-3-hydroxypiperidin-1-yl)-5-(1-(tetrahydro-2H-pyran-4-yl)-1H-pyrazol-4-yl)pyridin-2-yl)amino)nicotinic acid

Methyl 2-(2-fluoro-6-methoxyphenyl)-6-((4-((S)-3-hydroxypiperidin-1-yl)-5-(1-(tetrahydro-2H-pyran-4-yl)-1H-pyrazol-4-yl)pyridin-2-yl)amino)nicotinate (48.9 mg, 0.08 mmol) was dissolved in tetrahydrofuran (1.5 ml) and methanol (0.5 ml) and then 1.0 M sodium hydroxide solution (0.24 mL, 0.24 mmol) was added dropwise thereto. Then, the mixed solution was stirred at 60 °C overnight. After completion of the reaction, the reaction mixture was cooled to room temperature and then neutralized with 1.0 N hydrochloric acid solution. The aqueous layer was extracted with dichloromethane. The extracted organic layer was dried over anhydrous magnesium sulfate and then filtered. The filtrate was concentrated and then dried to prepare the titled compound (33.0 mg), without further purification. (Yield: 69.0%) 1H NMR (CD₃OD, 400 MHz) δ 8.43 (d, 1H), 8.03 (s, 1H), 7.88 (s, 1H), 7.74 (s, 1H), 7.46-7.44 (m, 1H), 7.15 (d, 1H), 6.96 (d, 1H), 6.87 (t, 1H), 6.78 (s, 1H), 4.49-4.46 (m, 1H), 4.09 (d, 2H), 3.80 (s, 4H), 3.63-3.56 (m, 2H), 3.48 (d, 1H), 3.14-3.07 (m, 1H), 2.94-2.89 (m, 1H), 2.16-2.06 (m, 4H), 2.02-1.98 (m, 1H), 1.88-1.84 (m, 1H), 1.62-1.49 (m, 2H)

### Step 3: 2-(2-fluoro-6-methoxyphenyl)-6-((4-((S)-3-hydroxypiperidin-1-yl)-5-(1-(tetrahydro-2H-pyran-4-yl)-1H-pyrazol-4-yl)pyridin-2-yl)amino)nicotinamide

2-(2-Fluoro-6-methoxyphenyl)-6-((4-((S)-3-hydroxypiperidin-1-yl)-5-(1-(tetrahydro-2H-pyran-4-yl)-1H-pyrazol-4-yl)pyridin-2-yl)amino)nicotinic acid (10.0 mg, 0.017 mmol) was dissolved in anhydrous dimethylformamide (1.0 mL) and then 1-[bis(dimethylamino)methylene]-1H-1,2,3-triazolo[4,5-b]pyridinium-3-oxide hexafluorophosphate (HATU, 12.92 mg, 0.034 mmol) and diisopropylethylamine (0.01 mL, 0.051 mmol) were added dropwise thereto. The reaction mixture was stirred at room temperature for 15 minutes and then 0.4M ammonia solution dissolved in 1,4-dioxane (0.05 mL, 0.02 mmol) was dropwise thereto. The reaction mixture was stirred at room temperature overnight. Water was added to the reaction mixture and then the aqueous layer was extracted with ethyl acetate. The extracted organic layer was dried over anhydrous magnesium sulfate and then filtered. The filtrate was concentrated and then purified by silica gel column chromatography to prepare the titled compound (2.3 mg). (Yield: 23.0%) 1H NMR (CD₃OD, 400 MHz) δ 8.08 (s, 1H), 8.03 (s, 1H), 7.96 (d, 1H), 7.85 (d, 1H), 7.82 (s, 1H), 7.41-7.33 (m, 2H), 6.91-6.83 (m, 2H), 4.49-4.45 (m, 1H), 4.09 (d, 2H), 3.80 (s, 3H), 3.70-3.57 (m, 3H), 3.00-2.94 (m, 1H), 2.64-2.55 (m, 1H), 2.41-2.37 (m, 1H), 2.20-2.04 (m, 4H), 1.97-1.92 (m, 1H), 1.79-1.72 (m, 1H), 1.55-1.50 (m, 1H), 1.35-1.30 (m, 1H) MS m/z=588.3 (M+H)+

### Example 113: 2-(2-fluoro-6-methoxyphenyl)-6-((4-((S)-3-hydroxypiperidin-1-yl)-5-(1-(tetrahydro-2H-pyran-4-yl)-1H-pyrazol-4-yl)pyridin-2-yl)amino)-N,N-dimethylnicotinamide

The titled compound (4.3 mg) was prepared in accordance with the same procedures as in Example 112, using Intermediates A2-1 and C2-1 and changing the amine reagent to 2.0M dimethylamine dissolved in tetrahydrofuran (0.01 mL, 0.02 mmol). (Yield: 41.1%) 1H NMR (CD₃OD, 400 MHz) δ 8.08 (s, 1H), 8.02 (s, 1H), 7.85 (d, 1H), 7.82 (s, 1H), 7.71 (d, 1H), 7.42-7.37 (m, 2H), 6.91 (d, 1H), 6.87-4.45 (m, 1H), 4.48-4.46 (m, 1H), 4.09 (d, 2H), 3.78 (s, 3H), 3.72-3.70 (m, 1H), 3.60 (t, 2H), 3.05-3.00 (m, 1H), 2.89 (s, 3H), 2.85 (s, 3H), 2.62-2.58 (m, 1H), 2.40 (t, 1H), 2.14-2.02 (m, 4H), 1.95-1.92 (m, 1H), 1.73-1.72 (m, 1H), 1.60-1.50 (m, 1H), 1.34-1.30 (m, 2H) MS m/z=616.3 (M+H)+

### Example 114: 4-((S)-3-aminopiperidin-1-yl)-N-(5-fluoro-6-(2-fluoro-6-methoxyphenyl)pyridin-2-yl)-5-((1-(trifluoromethyl)-1H-pyrazol-4-yl)ethynyl)pyridin-2-amine hydrochloride

The titled compound (18.0 mg) was prepared in accordance with the same procedures as in Example 52, using Intermediates A7-2 and C1-2. (Yield: 52.8%) 1H NMR (CD₃OD, 400 MHz) δ 8.6 (s, 1H), 8.25 (s, 1H), 8.04 (s, 1H), 7.88-7.84 (m, 1H), 7.55-7.53 (m, 1H), 7.31 (d, 1H), 7.07 (d, 1H), 6.94 (dd, 1H), 6.75 (s, 1H), 4.33-4.30 (m, 1H), 4.19-4.16 (m, 1H), 3.88 (s, 3H), 3.51-3.49 (m, 1H), 3.39-3.37 (m, 2H), 2.27-2.24 (m, 1H), 2.05-2.02 (m, 1H), 1.89-1.81 (m, 1H), 1.78-1.76 (m, 1H)

### Example 115: (S)-N-(4-(3-aminopiperidin-1-yl)-5-((1-(trifluoromethyl)-1H-pyrazol-4-yl)ethynyl)pyridin-2-yl)-2-(7-fluoroquinoxalin-6-yl)pyrimidin-4-amine hydrochloride

The titled compound (5.0 mg) was prepared in accordance with the same procedures as in Example 52, using Intermediates A7-2 and C1-6. (Yield: 14.6%) 1H NMR (CD₃OD, 400 MHz) δ 9.08 (d, 1H), 8.99-8.98 (m, 2H), 8.85 (d, 1H), 8.64 (s, 1H), 8.45 (s, 1H), 8.08 (s, 1H), 8.05 (d, 1H), 7.30 (s, 1H), 6.98 (s, 1H), 4.40-4.37 (m, 1H), 4.25-4.22 (m, 1H), 3.57-3.52 (m, 1H), 3.48-3.37 (m, 2H), 2.28-2.26 (m, 1H), 2.08-2.07 (m, 1H), 2.05-2.02 (m, 1H), 1.88-1.76 (m, 1H)

### Example 116: (S)-N-(4-(3-aminopiperidin-1-yl)-5-(1-(3-methoxypropyl)-1H-pyrazol-4-yl)pyridin-2-yl)-2-(2-fluoro-6-methoxyphenyl)pyrimidin-4-amine hydrochloride

The titled compound (8.2 mg) was prepared in accordance with the same procedures as in Example 52, using Intermediates A3-6 and C1-1. (Yield: 96.6%) 1H NMR (CD₃OD, 400 MHz) δ 8.82 (d, 1H), 8.14 (d, 2H), 7.86 (s, 1H), 7.74 (dd, 1H), 7.57 (d, 1H), 7.35 (s, 1H), 7.18 (d, 1H), 7.05 (t, 1H), 4.34 (t, 2H), 3.99 (s, 3H), 3.96 (s, 1H), 3.67 (d, 1H), 3.42 (t, 3H), 3.35-3.32 (m, 3H), 3.10 (t, 1H), 3.02 (t, 1H), 2.22-2.14 (m, 3H), 1.82 (brs, 1H), 1.75-1.65 (m, 2H). MS m/z=533.3 (M+H)+

### Example 117: 2-(2-fluoro-6-methoxyphenyl)-N-(4-(2-methyl-2,8-diazaspiro[4.5]decan-8-yl)-5-(1-(tetrahydro-2H-pyran-4-yl)-1H-pyrazol-4-yl)pyridin-2-yl)pyrimidin-4-amine

The titled compound (22.1 mg) was prepared in accordance with the same procedures as in Example 7, using Intermediates B3-23 and C1-1. (Yield: 40.5%) 1H NMR (CD₃OD, 400 MHz) δ 8.41 (d, 1H), 8.08 (s, 1H), 8.06 (s, 1H), 7.86 (s, 1H), 7.77 (s, 1H), 7.48-7.40 (m, 2H), 6.97 (d, 1H), 6.86 (t, 1H), 4.54-4.42 (m, 1H), 4.09 (d, 2H), 3.82 (s, 3H), 3.60 (t, 2H), 2.92-2.82 (m, 4H), 2.80-2.66 (m, 2H), 2.56-2.50 (m, 2H), 2.53 (s, 3H), 2.14-2.02 (m, 4H), 1.72 (t, 2H), 1.72-1.61 (m, 4H) MS m/z=599.3 (M+H)+

### Example 118: (S)-4-(3-aminopiperidin-1-yl)-N⁶-(2-(2-fluoro-6-methoxyphenyl)pyrimidin-4-yl)-N^{6′},N^{6′}-dimethyl-[3,3′-bipyridin]-6,6′-diamine hydrochloride

The titled compound (30.1 mg) was prepared in accordance with the same procedures as in Example 52, using Intermediates A3-16 and C1-1. (Yield: 97.3%) 1H NMR (CD₃OD, 400 MHz) δ 8.68 (d, 1H), 8.26 (d, 1H), 8.21 (s, 1H), 8.17 (s, 1H), 7.63 (q, 1H), 7.38 (d, 1H), 7.29 (s, 1H), 7.11 (d, 1H), 6.89 (t, 1H), 3.93 (s, 3H), 3.73 (d, 1H), 3.48-3.30 (m, 2H), 3.39 (s, 6H), 2.98-2.80 (m, 2H), 2.22-2.10 (m, 1H), 1.92-1.80 (m, 1H), 1.80-1.65 (m, 1H), 1.65-1.50 (m, 1H) MS m/z=515.1 (M+H)+

### Example 119: (S)-4-(3-aminopiperidin-1-yl)-N-(2-(2-fluoro-6-methoxyphenyl)pyrimidin-4-yl)-6′-(pyrrolidin-1-yl)-[3,3′-bipyridin]-6-amine hydrochloride

The titled compound (20.7 mg) was prepared in accordance with the same procedures as in Example 52, using Intermediates A3-17 and C1-1. (Yield: 97.1%) 1H NMR (CD₃OD, 400 MHz) δ 8.56 (d, 1H), 8.17 (d, 1H), 8.11 (s, 1H), 8.10 (s, 1H), 7.66 (s, 1H), 7.55-7.45 (m, 2H), 7.13 (d, 1H), 7.03 (d, 1H), 6.91 (t, 1H), 3.87 (s, 3H), 3.76-3.65 (m, 4H), 3.65-3.55 (m, 1H), 3.40-3.20 (m, 2H), 2.88-2.65 (m, 2H), 2.26-2.15 (m, 4H), 2.15-2.00 (m, 1H), 1.90-1.80 (m, 1H), 1.80-1.65 (m, 1H), 1.65-1.45 (m, 1H) MS m/z=540.9 (M+H)+

### Example 120: (S)-4-(3-aminopiperidin-1-yl)-N-(2-(2-fluoro-6-methoxyphenyl)pyrimidin-4-yl)-6′-(piperidin-1-yl)-[3,3′-bipyridin]-6-amine hydrochloride

The titled compound (29.0 mg) was prepared in accordance with the same procedures as in Example 52, using Intermediates A3-18 and C1-1. (Yield: 96.8%) 1H NMR (CD₃OD, 400 MHz) δ 8.68 (d, 1H), 8.23 (d, 1H), 8.20 (s, 1H), 8.16 (s, 1H), 7.63 (q, 1H), 7.49 (d, 1H), 7.28 (s, 1H), 7.11 (d, 1H), 6.98 (t, 1H), 3.80 (s, 3H), 3.84-3.75 (m, 4H), 3.75-3.70 (m, 1H), 3.44 (d, 1H), 3.42-3.32 (m, 1H), 2.88 (q, 2H), 2.18 (d, 1H), 1.90-1.78 (m, 7H), 1.74 (q, 1H), 1.57 (q, 1H) MS m/z=556.0 (M+H)+

### Example 121: (S)-4-(3-aminopiperidin-1-yl)-N-(2-(2-fluoro-6-methoxyphenyl)pyrimidin-4-yl)-6′-morpholino-[3,3′-bipyridin]-6-amine hydrochloride

The titled compound (24.0 mg) was prepared in accordance with the same procedures as in Example 52, using Intermediates A3-19 and C1-1. (Yield: 96.4%) 1H NMR (CD₃OD, 400 MHz) δ 8.69 (d, 1H), 8.25 (s, 1H), 8.25-8.18 (m, 2H), 7.62 (q, 1H), 7.42 (d, 1H), 7.26 (s, 1H), 7.11 (d, 1H), 6.98 (t, 1H), 3.93 (s, 3H), 3.93-3.84 (m, 4H), 3.80-3.68 (m, 5H), 3.52-3.30 (m, 2H), 2.87 (q, 2H), 2.17 (d, 1H), 1.90-1.80 (m, 1H), 1.73 (q, 1H), 1.57 (q, 1H) MS m/z=557.9 (M+H)+

### Example 122: (S)-N-(5-(1-(difluoromethyl)-1H-pyrazol-4-yl)-4-(3-(methylamino)piperidin-1-yl)pyridin-2-yl)-2-(2-fluoro-6-methoxyphenyl)pyrimidin-4-amine hydrochloride

The titled compound (7.0 mg) was prepared in accordance with the same procedures as in Example 52, using Intermediates B1-1 and C1-1. (Yield: 97.6%) 1H NMR (CD₃OD, 400 MHz) δ 8.77 (d, 1H), 8.54 (s, 1H), 8.23 (s, 1H), 8.1 (s, 1H), 7.78-7.49 (m, 2H), 7.29 (s, 1H), 7.15 (d, 1H), 7.03-6.99 (m, 1H), 3.95 (s, 3H), 3.91-3.89 (m, 1H), 3.55-3.52 (m, 1H), 3.33-3.32 (m, 1H), 3.04-2.94 (m, 2H), 2.61 (s, 3H), 2.30-2.27 (m, 1H), 1.92-1.89 (m, 1H), 1.85-1.77 (m, 1H), 1.64-1.61 (m, 1H) MS m/z=524.9 (M+H)+

### Example 123: (R)-N-(5-(1-(difluoromethyl)-1H-pyrazol-4-yl)-4-(3-(methylamino)piperidin-1-yl)pyridin-2-yl)-2-(2-fluoro-6-methoxyphenyl)pyrimidin-4-amine hydrochloride

The titled compound (15.3 mg) was prepared in accordance with the same procedures as in Example 52, using Intermediates B1-2 and C1-1. (Yield: 81.2%) 1H NMR (CD₃OD, 400 MHz) δ 8.79 (d, 1H), 8.55 (s, 1H), 8.23 (s, 1H), 8.11 (s, 1H), 7.79-7.49 (m, 2H), 7.32 (s, 1H), 7.17 (d, 1H), 7.14 (dd, 1H), 3.97 (s, 3H), 3.93-3.92 (m, 1H), 3.59-3.56 (m, 1H), 3.33-3.32 (m, 1H), 3.05-2.97 (m, 2H), 2.62 (s, 3H), 2.30-2.27 (m, 1H), 1.92-1.89 (m, 1H), 1.77-1.74 (m, 1H), 1.68-1.63 (m, 1H) MS m/z=524.9 (M+H)+

### Example 124: (S)-2-(2-fluoro-6-methoxyphenyl)-N-(4-(3-(methylamino)pyrrolidin-1-yl)-5-(1-(tetrahydro-2H-pyran-4-yl)-1H-pyrazol-4-yl)pyridin-2-yl)pyrimidin-4-amine

The titled compound (5.2 mg) was prepared in accordance with the same procedures as in Example 7, using Intermediates B3-24 and C1-1. (Yield: 21.3%) 1H NMR (CD₃OD, 400 MHz) δ 8.40 (d, 1H), 7.81 (s, 1H), 7.79 (s, 1H), 7.57 (s, 1H), 7.50-7.34 (m, 3H), 6.97 (d, 1H), 6.85 (t, 1H), 4.52-4.40 (m, 1H), 4.10 (d, 2H), 3.83 (s, 3H), 3.61 (t, 2H), 3.30-3.18 (m, 3H), 3.10 (q, 1H), 3.00-2.90 (m, 1H), 2.32 (s, 3H), 2.20-2.00 (m, 5H), 1.80-1.68 (m, 1H) MS m/z=545.9 (M+H)+

### Example 125: (S)-N-(4-(3-(ethylamino)pyrrolidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine

The titled compound (11.9 mg) was prepared in accordance with the same procedures as in Example 7, using Intermediates B3-25 and C1-1. (Yield: 18.6%) 1H NMR (CD₃OD, 400 MHz) δ 8.40 (d, 1H), 7.79 (s, 1H), 7.78 (s, 1H), 7.56 (s, 1H), 7.50-7.35 (m, 3H), 6.97 (d, 1H), 6.85 (t, 1H), 4.52-4.40 (m, 1H), 4.10 (d, 2H), 3.83 (s, 3H), 3.61 (t, 2H), 3.29-3.18 (m, 3H), 3.10 (q, 1H), 2.94-2.84 (m, 1H), 2.60-2.42 (m, 2H), 2.20-2.00 (m, 5H), 1.80-1.64 (m, 1H), 1.10 (t, 3H) MS m/z=559.9 (M+H)+

### Example 126: (R)-N-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)pyrrolidin-3-yl)acetamide

The titled compound (13.2 mg) was prepared in accordance with the same procedures as in Example 7, using Intermediates B3-26 and C1-1. (Yield: 39.0%) 1H NMR (CD₃OD, 400 MHz) δ 8.41 (d, 1H), 7.81 (s, 1H), 7.78 (s, 1H), 7.55 (s, 1H), 7.50-7.40 (m, 2H), 7.39 (d, 1H), 6.97 (d, 1H), 6.85 (t, 1H), 4.52-4.40 (m, 1H), 4.34-4.24 (m, 1H), 4.10 (d, 2H), 3.83 (s, 3H), 3.61 (t, 2H), 3.31-3.28 (m, 1H), 3.28-3.19 (m, 1H), 3.19-3.10 (m, 1H), 2.96-2.85 (m, 1H), 2.20-2.00 (m, 5H), 1.92 (s, 3H), 1.88-1.74 (m, 1H) MS m/z=573.9 (M+H)+

### Example 127: (S)-3-(6-((4-(3-aminopiperidin-1-yl)-5-(1-(tetrahydro-2H-pyran-4-yl)-1H-pyrazol-4-yl)pyridin-2-yl)amino)pyridin-2-yl)oxazolidin-2-one hydrochloride

The titled compound (20.3 mg) was prepared in accordance with the same procedures as in Example 52, using Intermediates A3-1 and C7. (Yield: 64.8%) 1H NMR (CD₃OD, 400 MHz) δ 8.13 (s, 1H), 7.99 (s, 1H), 7.78 (s, 1H), 7.55 (s, 1H), 7.50-7.40 (m, 2H), 7.39 (d, 1H), 6.97 (d, 1H), 6.85 (t, 1H), 4.52-4.40 (m, 1H), 4.34-4.24 (m, 1H), 4.10 (d, 2H), 3.83 (s, 3H), 3.61 (t, 2H), 3.31-3.28 (m, 1H), 3.28-3.18 (m, 1H), 3.18-3.08 (m, 1H), 2.96-2.86 (m, 1H), 2.20-2.00 (m, 5H), 1.92 (s, 3H), 1.88-1.76 (m, 1H) MS m/z=505.9 (M+H)+

### Example 128: (S)-N-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)pyrrolidin-3-yl)acetamide

The titled compound (4.5 mg) was prepared in accordance with the same procedures as in Example 7, using Intermediates B3-27 and C1-1. (Yield: 14.3%) 1H NMR (CD₃OD, 400 MHz) δ 8.40 (d, 1H), 7.81 (s, 1H), 7.96-7.80 (m, 2H), 7.00-6.84 (m, 2H), 4.68-4.58 (m, 3H), 4.58-4.48 (m, 1H), 4.26 (t, 2H), 4.11 (d, 2H), 3.80-3.70 (m, 1H), 3.62 (t, 2H), 3.50-3.35 (m, 2H), 2.95-2.78 (m, 2H), 2.28-2.09 (m, 4H), 1.90-1.78 (m, 2H), 1.78-1.50 (m, 1H) MS m/z=573.9 (M+H)+

### Example 129: 6-((4-((S)-3-aminopiperidin-1-yl)-5-(1-(tetrahydro-2H-pyran-4-yl)-1H-pyrazol-4-yl)pyridin-2-yl)amino)-2-(2-fluoro-6-methoxyphenyl)nicotinamide hydrochloride

### Step 1: methyl 6-((4-((S)-3-((tert-butoxycarbonyl)amino)piperidin-1-yl)-5-(1-(tetrahydro-2H-pyran-4-yl)-1H-pyrazol-4-yl)pyridin-2-yl)amino)-2-(2-fluoro-6-methoxyphenyl)nicotinate

The titled compound (58.3 mg) was prepared in accordance with the same procedures as in Step 1 of Example 52, using Intermediates A3-1 and C2-1. (Yield: 32.8%) 1H NMR (CDCl₃, 400 MHz) δ 8.28-8.25 (m, 1H), 8.10 (d, 1H), 7.80 (s, 1H), 7.77 (s, 1H), 7.62-7.60 (m, 2H), 7.37-7.34 (m, 2H), 6.88-6.77 (m, 2H), 4.54-4.39 (m, 2H), 4.17-4.14 (m, 2H), 3.76-3.72 (m, 6H), 3.63-3.56 (m, 2H), 3.31-3.22 (m, 1H), 3.01-2.94 (m, 1H), 2.54-2.48 (m, 1H), 2.43-2.39 (m, 1H), 2.24-2.07 (m, 4H), 1.97-1.90 (m, 1H), 1.48 (s, 9H).

### Step 2: 6-((4-((S)-3-((tert-butoxycarbonyl)amino)piperidin-1-yl)-5-(1-(tetrahydro-2H-pyran-4-yl)-1H-pyrazol-4-yl)pyridin-2-yl)amino)-2-(2-fluoro-6-methoxyphenyl)nicotinic acid

Methyl 6-((4-((S)-3-((tert-butoxycarbonyl)amino)piperidin-1-yl)-5-(1-(tetrahydro-2H-pyran-4-yl)-1H-pyrazol-4-yl)pyridin-2-yl)amino)-2-(2-fluoro-6-methoxyphenyl) nicotinate (68.0 mg, 0.09 mmol) was dissolved in tetrahydrofuran (1.5 ml) and methanol (0.5 ml) and then 1.0 M sodium hydroxide solution (0.29 mL, 0.24 mmol) was added dropwise thereto. Then, the mixed solution was stirred at 60 °C overnight. After completion of the reaction, the reaction mixture was cooled to room temperature and then neutralized with 1.0 N hydrochloric acid solution. The aqueous layer was extracted with dichloromethane. The extracted organic layer was dried over anhydrous magnesium sulfate and then filtered. The filtrate was concentrated and then dried to prepare the titled compound (57.1 mg), without further purification. (Yield: 85.8%) 1H NMR (CD₃OD, 400 MHz) δ 8.44-8.41 (m, 1H), 8.01 (d, 1H), 7.90 (s, 1H), 7.73 (s, 1H), 7.46-7.44 (m, 1H), 7.17 (d, 1H), 6.88 (t, 2H), 4.49-4.45 (m, 1H), 4.09 (d, 2H), 3.81 (s, 3H), 3.75-3.71 (m, 1H), 3.65-3.57 (m, 3H), 3.52-3.48 (m, 1H), 2.96-2.86 (m, 1H), 2.72-2.65 (m, 1H), 2.18-2.10 (m, 4H), 2.03-1.98 (m, 1H), 1.76-1.72 (m, 1H), 1.62-1.55 (m, 1H), 1.46 (s, 9H)

### Step 3: tert-butyl ((3S)-1-(2-((5-carbamoyl-6-(2-fluoro-6-methoxyphenyl)pyridin-2-yl)amino)-5-(1-(tetrahydro-2H-pyran-4-yl)-1H-pyrazol-4-yl)pyridin-4-yl)piperidin-3-yl)carbamate

6-((4-((S)-3-((tert-Butoxycarbonyl)amino)piperidin-1-yl)-5-(1-(tetrahydro-2H-pyran-4-yl)-1H-pyrazol-4-yl)pyridin-2-yl)amino)-2-(2-fluoro-6-methoxyphenyl)nicotinic acid (27.0 mg, 0.039 mmol) was dissolved in anhydrous dimethylformamide (1.0 mL) and then 1-[bis(dimethylamino)methylene]-1H-1,2,3-triazolo[4,5-b]pyridinium-3-oxide hexafluorophosphate (HATU, 29.8 mg, 0.079 mmol) and diisopropylethylamin e (0.02 mL, 0.12 mmol) were added dropwise thereto. The reaction mixture was stirred at room temperature for 15 minutes and then 0.4M ammonia solution dissolved in 1,4-dioxane (0.12 mL, 0.047 mmol) was added dropwise thereto. The reaction mixture was stirred at room temperature overnight. Water was added to the reaction mixture and then the aqueous layer was extracted with ethyl acetate. The extracted organic layer was dried over anhydrous magnesium sulfate and then filtered. The filtrate was concentrated and then purified by silica gel column chromatography to prepare the titled compound (12.3 mg). (Yield: 45.6%) 1H NMR (CD₃OD, 400 MHz) δ 8.21-8.19 (m, 1H), 8.09 (d, 1H), 7.79-7.77 (m, 2H), 7.59-7.54 (m, 2H), 7.46-7.34 (m, 2H), 6.93-6.83 (m, 2H), 5.64 (brs, 1H), 5.32 (brs, 1H), 4.50-4.39 (m, 2H), 4.17-4.14 (m, 2H), 3.82 (s, 3H), 3.76-3.71 (m, 1H), 3.63-3.56 (m, 2H), 3.33-3.19 (m, 2H), 2.98-2.91 (m, 1H), 2.64-2.53 (m, 1H), 2.45-2.40 (m, 1H), 2.24-2.11 (m, 4H), 1.98-1.90 (m, 1H), 1.48 (s, 9H)

### Step 4: 6-((4-((S)-3-aminopiperidin-1-yl)-5-(1-(tetrahydro-2H-pyran-4-yl)-1H-pyrazol-4-yl)pyridin-2-yl)amino)-2-(2-fluoro-6-methoxyphenyl)nicotinamide hydrochloride

tert-Butyl ((3S)-1-(2-((5-carbamoyl-6-(2-fluoro-6-methoxyphenyl)pyridin-2-yl)amino)-5-(1-(tetrahydro-2H-pyran-4-yl)-1H-pyrazol-4-yl)pyridin-4-yl)piperidin-3-yl) carbamate (12.3 mg, 0.02 mmol) was dissolved in dichloromethane (1 ml) and then 4.0 M hydrogen chloride solution dissolved in 1,4-dioxane (0.5 ml, 7.69 mmol) was added dropwise thereto. Then, the mixed solution was stirred at room temperature for 4 hours. After completion of the reaction, the reaction mixture was concentrated and then filtered. The resulting solid was dried to prepare the titled compound (3.3 mg). (Yield: 27.9%) 1H NMR (CD₃OD, 400 MHz) δ 8.20 (d, 1H), 8.10 (s, 1H), 8.00 (s, 1H), 7.83 (s, 1H), 7.50-7.48 (m, 1H), 7.25-7.23 (m, 1H), 6.99 (d, 1H), 6.94-6.85 (m, 2H), 4.09 (d, 1H), 4.12-4.09 (m, 2H), 3.85 (s, 3H), 3.83-3.81 (m, 1H), 3.65-3.50 (m, 3H), 3.49-3.42 (m, 1H), 2.93-2.88 (m, 2H), 2.22-2.20 (m, 1H), 2.15-2.10 (m, 3H), 1.90-1.70 (m, 2H), 1.65-1.59 (m, 2H) MS m/z=587.9 (M+H)+

### Example 130: 6-((4-((S)-3-aminopiperidin-1-yl)-5-(1-(tetrahydro-2H-pyran-4-yl)-1H-pyrazol-4-yl)pyridin-2-yl)amino)-2-(2-fluoro-6-methoxyphenyl)-N,N-dimethylnicotinamide hydrochloride

The titled compound (3.3 mg) was prepared in accordance with the same procedures as in Example 129, using Intermediates A3-1 and C2-1 and changing the amine reagent to 2.0M dimethylamine dissolved in tetrahydrofuran (0.02 mL, 0.05 mmol). (Yield: 27.9%) 1H NMR (CD₃OD, 400 MHz) δ 8.10 (s, 1H), 8.09-8.00 (m, 2H), 7.82 (s, 1H), 7.83 (s, 1H), 7.52-7.48 (m, 1H), 7.29-7.25 (m, 1H), 7.01 (d, 1H), 6.93-6.88 (m, 2H), 4.58-4.52 (m, 1H), 4.12-4.09 (m, 2H), 3.85-3.82 (m, 4H), 3.64-3.54 (m, 4H), 3.50-3.43 (m, 1H), 2.95-2.91 (m, 4H), 2.88-2.85 (m, 4H), 2.24-2.20 (m, 1H), 2.15-2.09 (m, 4H), 1.90-1.72 (m, 2H), 1.66-1.60 (m, 1H) MS m/z=615.9 (M+H)+

### Example 131: (S)-N-(4-(3-aminopiperidin-1-yl)-5-(1-methyl-3-(trifluoromethyl)-1H-pyrazol-4-yl)pyridin-2-yl)-2-(2-fluoro-6-methoxyphenyl)pyrimidin-4-amine hydrochloride

The titled compound (19.4 mg) was prepared in accordance with the same procedures as in Example 52, using Intermediates A3-7 and C1-1. (Yield: 67.1%) 1H NMR (CD₃OD, 400 MHz) δ 8.70 (d, 1H), 8.05 (s, 1H), 8.03 (s, 1H), 7.60 (q, 1H), 7.18-7.06 (m, 2H), 6.96 (t, 1H), 4.06 (s, 3H), 3.91 (s, 3H), 3.80-3.68 (m, 1H), 3.52-3.40 (m, 1H), 3.30-3.20 (m, 1H), 2.86 (q, 1H), 2.22-2.10 (m, 1H), 2.10-2.00 (m, 1H), 1.84-1.75 (m, 1H), 1.80-1.50 (m, 1H), 1.00-0.82 (m, 1H) MS m/z=543.9 (M+H)+

### Example 132: (S)-2-(4-(4-(3-aminopiperidin-1-yl)-6-((2-(2-fluoro-6-methoxyphenyl)pyrimidin-4-yl)amino)pyridin-3-yl)-1H-pyrazol-1-yl)-N,N-dimethylacetamide hydrochloride

The titled compound (7.8 mg) was prepared in accordance with the same procedures as in Example 52, using Intermediates A3-8 and C1-1. (Yield: 54.2%) 1H NMR (CD₃OD, 400 MHz) δ 8.45 (d, 1H), 8.11 (s, 1H), 7.97 (s, 1H), 7.79 (s, 1H), 7.74 (s, 1H), 7.52 (d, 1H), 7.47 (q, 1H), 6.99 (d, 1H), 6.86 (t, 1H), 5.26 (q, 2H), 3.84 (s, 3H), 3.50 (d, 1H), 3.30-3.20 (m, 1H), 3.19 (s, 3H), 3.03 (s, 3H), 2.82 (t, 1H), 2.48 (t, 1H), 2.05 (q, 1H), 1.98-1.85 (m, 1H), 1.85-1.70 (m, 1H), 1.56-1.42 (m, 1H), 1.00-0.82 (m, 1H) MS m/z=547.0 (M+H)+

### Example 133: (S)-2-(4-(4-(3-aminopiperidin-1-yl)-6-((2-(2-fluoro-6-methoxyphenyl)pyrimidin-4-yl)amino)pyridin-3-yl)-1H-pyrazol-1-yl)ethan-1-ol hydrochloride

The titled compound (21.7 mg) was prepared in accordance with the same procedures as in Example 52, using Intermediates A3-9 and C1-1. (Yield: 52.8%) 1H NMR (CD₃OD, 400 MHz) δ 8.73 (d, 1H), 8.16 (s, 1H), 8.09 (s, 1H), 7.85 (s, 1H), 7.63 (q, 1H), 7.63-7.40 (m, 1H), 7.16 (s, 1H), 7.11 (d, 1H), 6.98 (t, 1H), 4.33 (t, 2H), 3.96 (t, 2H), 3.83 (s, 3H), 3.82 (d, 1H), 3.55 (d, 1H), 3.46-3.35 (m, 1H), 2.95 (t, 1H), 2.84 (t, 1H), 2.18 (d, 1H), 1.96-1.72 (m, 2H), 1.60 (q, 1H) MS m/z=506.0 (M+H)+

### Example 134: (S)-N-(4-(3-aminopiperidin-1-yl)-5-(1-(2,2-difluoroethyl)-1H-pyrazol-4-yl)pyridin-2-yl)-2-(2-fluoro-6-methoxyphenyl)pyrimidin-4-amine hydrochloride

The titled compound (10.6 mg) was prepared in accordance with the same procedures as in Example 52, using Intermediates A3-10 and C1-1. (Yield: 38.2%) 1H NMR (CD₃OD, 400 MHz) δ 8.84-8.83 (m, 1H), 8.23-8.18 (m, 2H), 7.91 (s, 1H), 7.77-7.71 (m, 1H), 7.59 (s, 1H), 7.36 (s, 1H), 7.20 (d, 1H), 7.08 (dd, 1H), 6.43-6.16 (m, 1H), 4.74-4.70 (m, 2H), 4.00 (s, 3H), 3.92-3.82 (m, 1H), 3.70-3.66 (m, 1H), 3.45-3.36 (m, 1H), 3.09-3.04 (m, 2H), 2.23-2.20 (m, 1H), 1.84-1.68 (m, 3H) MS m/z=525.2 (M+H)+

### Example 135: N-(5-(1-(difluoromethyl)-1H-pyrazol-4-yl)-4-(2-methyl-2,8-diazaspiro[4.5]decan-8-yl)pyridin-2-yl)-2-(2-fluoro-6-methoxyphenyl)pyrimidin-4-amine

The titled compound (14.4 mg) was prepared in accordance with the same procedures as in Example 7, using Intermediates B3-28 and C1-1. (Yield: 24.2%) 1H NMR (CD₃OD, 400 MHz) δ 8.44 (d, 1H), 8.40 (s, 1H), 8.15 (s, 1H), 8.12 (s, 1H), 7.84 (s, 1H), 7.54 (t, 1H), 7.50-7.40 (m, 2H), 6.99 (d, 1H), 6.87 (t, 1H), 3.84 (s, 3H), 2.92-2.84 (m, 3H), 2.65 (t, 2H), 2.46 (s, 2H), 2.38 (s, 3H), 2.10-2.00 (m, 1H), 1.70 (t, 2H), 1.68-1.52 (m, 4H) MS m/z=565.9 (M+H)+

### Example 136: 2-(2-fluoro-6-methoxyphenyl)-N-(4-(2-methyl-2,8-diazaspiro[4.5]decan-8-yl)-5-(1-(2,2,2-trifluoroethyl)-1H-pyrazol-4-yl)pyridin-2-yl)pyrimidin-4-amine

The titled compound (13.8 mg) was prepared in accordance with the same procedures as in Example 7, using Intermediates B3-29 and C1-1. (Yield: 21.9%) 1H NMR (CD₃OD, 400 MHz) δ 8.43 (d, 1H), 8.13 (s, 1H), 8.10 (s, 1H), 7.95 (s, 1H), 7.82 (s, 1H), 7.52-7.40 (m, 2H), 6.99 (d, 1H), 6.87 (t, 1H), 5.02 (q, 2H), 3.84 (s, 3H), 2.95-2.80 (m, 3H), 2.65 (t, 2H), 2.46 (s, 2H), 2.38 (s, 3H), 2.10-2.00 (m, 1H), 1.69 (t, 2H), 1.66-1.50 (m, 4H) MS m/z=567.9 (M+H)+

### Example 137: N-(2-(2-fluoro-6-methoxyphenyl)pyrimidin-4-yl)-4-(2-methyl-2,8-diazaspiro[4.5]decan-8-yl)-6′-morpholino-[3,3′-bipyridin]-6-amine

The titled compound (13.4 mg) was prepared in accordance with the same procedures as in Example 7, using Intermediates B3-30 and C1-1. (Yield: 26.5%) 1H NMR (CD₃OD, 400 MHz) δ 8.43 (d, 1H), 8.32 (d, 1H), 7.92 (s, 1H), 7.84 (d, 1H), 7.75 (s, 1H), 7.50-7.42 (m, 2H), 6.99 (d, 1H), 6.92 (d, 1H), 6.87 (t, 1H), 3.88-3.78 (m, 7H), 3.53 (t, 4H), 2.94-2.84 (m, 3H), 2.65 (t, 2H), 2.45 (s, 2H), 2.38 (s, 3H), 2.10-2.00 (m, 1H), 1.67 (t, 2H), 1.60-1.45 (m, 4H) MS m/z=612.0 (M+H)+

### Example 138: (S)-3-(6-((4-(3-aminopiperidin-1-yl)-6′-morpholino-[3,3′-bipyridin]-6-yl)amino)pyridin-2-yl)oxazolidin-2-one hydrochloride

The titled compound (5.9 mg) was prepared in accordance with the same procedures as in Example 52, using Intermediates A3-19 and C7. (Yield: 63.0%) 1H NMR (CD₃OD, 400 MHz) δ 8.32 (d, 1H), 7.88 (s, 1H), 7.84 (d, 1H), 7.70-7.64 (m, 1H), 7.64-7.58 (m, 1H), 7.43 (s, 1H), 7.16 (d, 1H), 6.94 (d, 1H), 4.55 (t, 2H), 4.39 (t, 2H), 3.84 (t, 4H), 3.54 (t, 4H), 3.45-3.38 (m, 1H), 3.24-3.16 (m, 1H), 3.00-2.90 (m, 1H), 2.65-2.48 (m, 2H), 2.08-1.95 (m, 2H), 1.68-1.50 (m, 2H) MS m/z=517.9 (M+H)+

### Example 139: 2-(2-fluoro-6-methoxyphenyl)-6-((4-((S)-3-hydroxypiperidin-1-yl)-5-((tetrahydro-2H-pyran-4-yl)ethynyl)pyridin-2-yl)amino)nicotinic acid

### Step 1: methyl 2-(2-fluoro-6-methoxyphenyl)-6-((4-((S)-3-hydroxypiperidin-1-yl)-5-((tetrahydro-2H-pyran-4-yl)ethynyl)pyridin-2-yl)amino)nicotinate

The titled compound (20.3 mg) was prepared in accordance with the same procedures as in Example 7, using Intermediates A6-2 and C2-1. (Yield: 33.3%) 1H NMR (CD₃OD, 400 MHz) δ 8.20 (d, 1H), 8.02 (s, 1H), 7.78 (s, 1H), 7.42-7.32 (m, 2H), 6.91-6.83 (m, 2H), 4.07-4.01 (m, 1H), 3.97-3.92 (m, 2H), 3.75 (d, 3H), 3.67 (s, 3H), 3.60-3.54 (m, 2H), 3.45-3.42 (m, 1H), 2.98-2.92 (m, 1H), 2.83-2.73 (m, 1H), 2.65-2.59 (m, 1H), 2.01-1.93 (m, 3H), 1.81-1.72 (m, 3H), 1.66-1.55 (m, 2H), 1.45-1.35 (m, 1H)

### Step 2: 2-(2-fluoro-6-methoxyphenyl)-6-((4-((S)-3-hydroxypiperidin-1-yl)-5-((tetrahydro-2H-pyran-4-yl)ethynyl)pyridin-2-yl)amino)nicotinic acid

Methyl 2-(2-fluoro-6-methoxyphenyl)-6-((4-((S)-3-hydroxypiperidin-1-yl)-5-((tetrahydro-2H-pyran-4-yl)ethynyl)pyridin-2-yl)amino)nicotinate (20.3 mg, 0.036 mmol) was dissolved in tetrahydrofuran (1.0 ml) and methanol (0.3 ml) and then 1.0 M sodium hydroxide solution (0.11 mL, 0.11 mmol) was added dropwise thereto. Then, the mixed solution was stirred at 60 °C overnight. After completion of the reaction, the reaction mixture was cooled to room temperature and then neutralized with 1.0 N hydrochloric acid solution. The aqueous layer was extracted with dichloromethane. The extracted organic layer was dried over anhydrous magnesium sulfate and then filtered. The filtrate was concentrated and then dried to prepare the titled compound (19.0 mg), without further purification. (Yield: 96.5%) 1H NMR (CD₃OD, 400 MHz) δ 8.42 (d, 1H), 8.05 (s, 1H), 7.48-7.43 (m, 1H), 7.14 (d, 1H), 6.97 (d, 1H), 6.89 (t, 1H), 6.66 (s, 1H), 4.20-4.14 (m, 1H), 3.95-3.84 (m, 3H), 3.82 (s, 3H), 3.59-3.50 (m, 3H), 3.41-3.39 (m, 1H), 2.99-2.93 (m, 1H), 2.10-1.92 (m, 4H), 1.79-1.66 (m, 4H), 1.40-1.34 (m, 1H) MS m/z=546.9 (M+H)+

### Example 140: 2-(2-fluoro-6-methoxyphenyl)-6-((4-((S)-3-hydroxypiperidin-1-yl)-5-((tetrahydro-2H-pyran-4-yl)ethynyl)pyridin-2-yl)amino)nicotinamide

The titled compound (7.4 mg) was prepared in accordance with the same procedures as in Step 3 of Example 112, using the compound prepared in Example 139. (Yield: 49.4%) 1H NMR (CD₃OD, 400 MHz) δ 8.01 (s, 1H), 7.97 (d, 1H), 7.76 (s, 1H), 7.42-7.37 (m, 1H), 7.34-7.31 (m, 1H), 6.92-6.85 (m, 2H), 4.07-4.02 (m, 1H), 3.97-3.92 (m, 2H), 3.81 (s, 3H), 3.74-3.72 (m, 1H), 3.60-3.50 (m, 3H), 2.97-2.93 (m, 1H), 2.88-2.73 (m, 1H), 2.64-2.62 (m, 1H), 2.03-2.00 (m, 1H), 1.98-1.93 (m, 2H), 1.81-1.74 (m, 2H), 1.62-1.62 (m, 1H), 1.37-1.35 (m, 1H) MS m/z=546.3 (M+H)+

### Example 141: 6-((5-(1-(difluoromethyl)-1H-pyrazol-4-yl)-4-((S)-3-hydroxypiperidin-1-yl)pyridin-2-yl)amino)-2-(2-fluoro-6-methoxyphenyl)-N,N-dimethylnicotinamide

The titled compound (1.1 mg) was prepared in accordance with the same procedures as in Example 112, using Intermediates A2-8 and C2-1 and changing the amine reagent to 2.0M dimethylamine dissolved in tetrahydrofuran (0.01 mL, 0.02 mmol). (Yield: 5.47%) 1H NMR (CD₃OD, 400 MHz) δ 8.40 (s, 1H), 8.09 (d, 2H), 7.91 (d, 1H), 7.73 (d, 1H), 7.53 (s, 1H), 7.46-7.38 (m, 2H), 6.92 (d, 1H), 6.88-6.86 (m, 1H), 3.80 (s, 3H), 3.73-3.70 (m, 1H), 3.30-3.23 (m, 1H), 3.05-2.98 (m, 1H), 2.90 (s, 3H), 2.87 (s, 3H), 2.63-2.61 (m, 1H), 2.48-2.43 (m, 1H), 1.95-1.94 (m, 1H), 1.75-1.73 (m, 1H), 1.57-1.55 (m, 2H) MS m/z=582.2 (M+H)+

### Example 142: 6-((5-(1-(difluoromethyl)-1H-pyrazol-4-yl)-4-((S)-3-hydroxypiperidin-1-yl)pyridin-2-yl)amino)-2-(2-fluoro-6-methoxyphenyl)nicotinamide

The titled compound (1.3 mg) was prepared in accordance with the same procedures as in Example 112, using Intermediates A2-8 and C2-1. (Yield: 10.0%) 1H NMR (CD₃OD, 400 MHz) δ 8.43 (s, 1H), 8.13 (s, 2H), 8.02 (d, 1H), 7.94 (d, 1H), 7.56 (t, 1H), 7.48-7.40 (m, 2H), 6.97-6.88 (m, 2H), 3.84 (s, 3H), 3.76-3.74 (m, 1H), 3.30-3.26 (m, 1H), 3.04-3.02 (m, 1H), 2.71-2.61 (m, 1H), 2.48 (t, 1H), 2.01-1.97 (m, 1H), 1.80-1.77 (m, 1H), 1.59-1.58 (m, 1H), 1.44-1.43 (m, 1H) MS m/z=554.2 (M+H)+

### Example 143: 2-(2-fluoro-6-methoxyphenyl)-6-((4-((S)-3-hydroxypiperidin-1-yl)-5-(1-(2,2,2-trifluoroethyl)-1H-pyrazol-4-yl)pyridin-2-yl)amino)nicotinamide

The titled compound (3.0 mg) was prepared in accordance with the same procedures as in Example 112, using Intermediates A2-6 and C2-1. (Yield: 28.5%) 1H NMR (CD₃OD, 400 MHz) δ 8.13 (s, 1H), 8.05 (s, 2H), 7.98 (d, 1H), 7.94 (s, 1H), 7.86 (s, 1H), 7.44-7.36 (m, 2H), 6.93-6.86 (m, 2H), 5.03-4.97 (m, 2H), 3.81 (s, 3H), 3.74-3.68 (m, 1H), 3.26-3.22 (m, 1H), 3.02-2.96 (m, 1H), 2.64-2.56 (m, 1H), 2.47-2.41 (m, 1H), 1.94-1.93 (m, 1H), 1.77-1.69 (m, 1H), 1.57-1.52 (m, 1H), 1.40-1.33 (m, 1H) MS m/z=586.2 (M+H)+

### Example 144: 2-(2-fluoro-6-methoxyphenyl)-6-((4-((S)-3-hydroxypiperidin-1-yl)-5-(1-(2-methoxyethyl)-1H-pyrazol-4-yl)pyridin-2-yl)amino)nicotinamide

The titled compound (7.7 mg) was prepared in accordance with the same procedures as in Example 112, using Intermediates A2-9 and C2-1. (Yield: 51.4%) 1H NMR (CD₃OD, 400 MHz) δ 8.03-8.02 (m, 2H), 7.97 (d, 1H), 7.85 (d, 1H), 7.82 (s, 1H), 7.44-7.35 (m, 2H), 6.93-6.86 (m, 2H), 4.36 (t, 2H), 3.82-3.70 (m, 5H), 3.75-3.70 (m, 1H), 3.36 (s, 3H), 3.29-3.28 (m, 1H), 3.02-2.98 (m, 1H), 2.60-2.56 (m, 1H), 2.38 (t, 1H), 1.97-1.95 (m, 1H), 1.76-1.72 (m, 1H), 1.62-1.58 (m, 1H), 1.40-1.35 (m, 1H) MS m/z=562.0 (M+H)+

### Example 145: (3S)-1-(5-(1-(difluoromethyl)-1H-pyrazol-4-yl)-2-((6-(2-fluoro-6-methoxyphenyl)-5-nitropyridin-2-yl)amino)pyridin-4-yl)piperidin-3-ol

The titled compound (29.9 mg) was prepared in accordance with the same procedures as in Example 7, using Intermediates A2-8 and C3-1. (Yield: 35.4%) 1H NMR (CDCl₃, 400 MHz) δ 8.40 (d, 1H), 8.14 (d, 2H), 7.97 (s, 1H), 7.86 (s, 1H), 7.57 (s, 1H), 7.48-7.37 (m, 2H), 7.26 (t, 1H), 6.88 (t, 1H), 6.79 (d, 1H), 3.85-3.82 (m, 1H), 3.76 (s, 3H), 3.23-3.15 (m, 1H), 3.00-2.93 (m, 1H), 2.72-2.62 (m, 2H), 1.94-1.92 (m, 1H), 1.78-1.74 (m, 1H), 1.52-1.47 (m, 2H). MS m/z=556.2 (M+H)+

### Example 146: 4-((S)-3-aminopiperidin-1-yl)-5-(1-(difluoromethyl)-1H-pyrazol-4-yl)-N-(6-(2-fluoro-6-methoxyphenyl)-5-nitropyridin-2-yl)pyridin-2-amine hydrochloride

The titled compound (5.6 mg) was prepared in accordance with the same procedures as in Example 52, using Intermediates A3-2 and C3-1. (Yield: 58.4%) 1H NMR (CD₃OD, 400 MHz) δ 8.60 (d, 1H), 8.49 (s, 1H), 8.16-8.07 (m, 2H), 7.60 (t, 1H), 7.58-7.52 (m, 1H), 7.36 (d, 1H), 7.09 (s, 1H), 7.02-6.95 (m, 1H), 3.79-3.77 (m, 4H), 3.56-3.49 (m, 1H), 3.43-3.37 (m, 1H), 2.97-2.91 (m, 2H), 2.22-2.20 (m, 1H), 1.87-1.74 (m, 2H), 1.64-1.58 (m, 1H) MS m/z=555.2 (M+H)+

### Example 147: N-(5-(1-(difluoromethyl)-1H-pyrazol-4-yl)-4-(4-(2-(dimethylamino)ethyl)piperidin-1-yl)pyridin-2-yl)-2-(2-fluoro-6-methoxyphenyl)pyrimidin-4-amine

The titled compound (13.9 mg) was prepared in accordance with the same procedures as in Example 7, using Intermediates B3-31 and C1-1. (Yield: 18.0%) 1H NMR (CD₃OD, 400 MHz) δ 8.44 (d, 1H), 8.38 (s, 1H), 8.14 (s, 1H), 8.10 (s, 1H), 7.72 (s, 1H), 7.54 (t, 1H), 7.58-7.40 (m, 2H), 6.98 (d, 1H), 6.86 (t, 1H), 3.84 (s, 3H), 2.58 (t, 2H), 2.39 (t, 2H), 2.29 (s, 6H), 2.10-2.00 (m, 1H), 1.66 (d, 2H), 1.55-1.44 (m, 2H), 1.44-1.20 (m, 4H) MS m/z=568.0 (M+H)+

### Example 148: N-(4-(4-(2-(dimethylamino)ethyl)piperidin-1-yl)-5-(1-(2,2,2-trifluoroethyl)-1H-pyrazol-4-yl)pyridin-2-yl)-2-(2-fluoro-6-methoxyphenyl)pyrimidin-4-amine

The titled compound (6.0 mg) was prepared in accordance with the same procedures as in Example 7, using Intermediates B3-32 and C1-1. (Yield: 17.2%) 1H NMR (CD₃OD, 400 MHz) δ 8.43 (d, 1H), 8.11 (s, 1H), 8.10 (s, 1H), 7.94 (s, 1H), 7.70 (s, 1H), 7.52-7.40 (m, 2H), 6.98 (d, 1H), 6.86 (t, 1H), 5.02 (q, 2H), 3.83 (s, 3H), 2.56 (t, 2H), 2.46-2.32 (m, 2H), 2.29 (s, 6H), 2.10-2.00 (m, 1H), 1.64 (d, 2H), 1.54-1.42 (m, 2H), 1.42-1.20 (m, 4H) MS m/z=599.9 (M+H)+

### Example 149: 4-(4-(2-(dimethylamino)ethyl)piperidin-1-yl)-N-(2-(2-fluoro-6-methoxyphenyl)pyrimidin-4-yl)-6′-morpholino-[3,3′-bipyridin]-6-amine

The titled compound (16.6 mg) was prepared in accordance with the same procedures as in Example 7, using Intermediates B3-33 and C1-1. (Yield: 24.5%) 1H NMR (CD₃OD, 400 MHz) δ 8.43 (d, 1H), 8.30 (d, 1H), 7.91 (s, 1H), 7.83 (d, 1H), 7.67 (s, 1H), 7.52-7.40 (m, 2H), 6.98 (d, 1H), 6.93 (d, 1H), 6.86 (t, 1H), 3.90-3.78 (m, 7H), 3.54 (t, 4H), 2.55 (t, 2H), 2.36 (t, 2H), 2.27 (s, 6H), 2.10-2.00 (m, 1H), 1.57 (d, 2H), 1.50-1.40 (m, 2H), 1.40-1.10 (m, 4H) MS m/z=614.1 (M+H)+

### Example 150: (3S)-1-(2-((5-amino-6-(2-fluoro-6-methoxyphenyl)pyridin-2-yl)amino)-5-(1-(difluoromethyl)-1H-pyrazol-4-yl)pyridin-4-yl)piperidin-3-ol

### Step 1: (3S)-1-(5-(1-(difluoromethyl)-1H-pyrazol-4-yl)-2-((6-(2-fluoro-6-methoxy phenyl)-5-nitropyridin-2-yl)amino)pyridin-4-yl)piperidin-3-ol

The titled compound (29.9 mg) was prepared in accordance with the same procedures as in Example 7, using Intermediates A2-8 and C3-1. (Yield: 35.4%) 1H NMR (CDCl₃, 400 MHz) δ 8.40 (d, 1H), 8.14 (d, 2H), 7.97 (s, 1H), 7.86 (s, 1H), 7.57 (s, 1H), 7.48-7.37 (m, 2H), 7.26 (t, 1H), 6.88 (t, 1H), 6.79 (d, 1H), 3.85-3.82 (m, 1H), 3.76 (s, 3H), 3.23-3.15 (m, 1H), 3.00-2.93 (m, 1H), 2.72-2.62 (m, 2H), 1.94-1.92 (m, 1H), 1.78-1.74 (m, 1H), 1.52-1.47 (m, 2H)

### Step 2: (3S)-1-(2-((5-amino-6-(2-fluoro-6-methoxyphenyl)pyridin-2-yl)amino)-5-(1-(difluoromethyl)-1H-pyrazol-4-yl)pyridin-4-yl)piperidin-3-ol

(3S)-1-(5-(1-(Difluoromethyl)-1H-pyrazol-4-yl)-2-((6-(2-fluoro-6-methoxyphenyl)-5-nitropyridin-2-yl)amino)pyridin-4-yl)piperidin-3-ol (15.0 mg, 0.027 mmol) was dissolved in methanol (2.0 mL) and then Pd/C (15 mg, 0.14 mmol) was added dropwise thereto. The reaction mixture was stirred at room temperature for 3 hours under a pressure of a balloon filled with hydrogen gas. After completion of the reaction, the reaction mixture was filtered through a Celite filter. The filtrate was concentrated and then purified by silica gel column chromatography to prepare the titled compound (5.0 mg). (Yield: 35.2%) 1H NMR (CD₃OD, 400 MHz) δ 8.36 (s, 1H), 8.06 (s, 1H), 7.97 (s, 1H), 7.52 (t, 1H), 7.49-7.37 (m, 1H), 7.31 (d, 2H), 7.25-7.22 (m, 1H), 6.99 (d, 1H), 6.90 (t, 1H), 3.86 (s, 3H), 3.75-3.71 (m, 1H), 3.28-3.25 (m, 1H), 3.11-3.02 (m, 1H), 2.68-2.62 (m, 1H), 2.50-2.46 (m, 1H), 1.98-1.94 (m, 1H), 1.77-1.74 (m, 1H), 1.65-1.52 (m, 1H), 1.38-1.36 (m, 1H) MS m/z=526.2 (M+H)+

### Example 151: N²-(4-((S)-3-aminopiperidin-1-yl)-5-(1-(difluoromethyl)-1H-pyrazol-4-yl)pyridin-2-yl)-6-(2-fluoro-6-methoxyphenyl)pyridin-2,5-diamine hydrochloride

### Step 1: tert-butyl ((3S)-1-(5-(1-(difluoromethyl)-1H-pyrazol-4-yl)-2-((6-(2-fluoro-6-methoxyphenyl)-5-nitropyridin-2-yl)amino)pyridin-4-yl)piperidin-3-yl)carbamate

The titled compound (45.7 mg) was prepared in accordance with the same procedures as in Step 1 of Example 52, using Intermediates A3-2 and C3-1. (Yield: 45.9%) 1H NMR (CDCl₃, 400 MHz) δ 8.42-8.38 (m, 1H), 8.12-8.11 (m, 2H), 7.96 (s, 1H), 7.85 (s, 1H), 7.45-7.40 (m, 2H), 7.26 (t, 1H), 6.92-6.89 (m, 1H), 6.81-6.78 (m, 1H), 4.47 (brs, 1H), 3.76 (s, 4H), 3.39 (d, 1H), 3.29 (d, 1H), 3.01-2.98 (m, 1H), 2.55-2.42 (m, 2H), 1.97-1.94 (m, 1H), 1.49 (s, 10H)

### Step 2: tert-butyl ((3S)-1-(2-((5-amino-6-(2-fluoro-6-methoxyphenyl)pyridin-2-yl)amino)-5-(1-(difluoromethyl)-1H-pyrazol-4-yl)pyridin-4-yl)piperidin-3-yl)carbamate

tert-Butyl ((3S)-1-(5-(1-(difluoromethyl)-1H-pyrazol-4-yl)-2-((6-(2-fluoro-6-methoxyphenyl)-5-nitropyridin-2-yl)amino)pyridin-4-yl)piperidin-3-yl)carbamate (17.7 mg, 0.03 mmol) was dissolved in methanol (2.0 mL) and then Pd/C (18.0 mg, 0.17 mmol) was added dropwise thereto. The reaction mixture was stirred at room temperature for 3 hours under a pressure of a balloon filled with hydrogen gas. After completion of the reaction, the reaction mixture was filtered through a Celite filter. The filtrate was concentrated and then purified by silica gel column chromatography to prepare the titled compound (9.90 mg). (Yield: 58.7%) 1H NMR (CD₃OD, 400 MHz) δ 8.32 (s, 1H), 8.03 (s, 1H), 7.96 (s, 1H), 7.52 (t, 1H), 7.50-7.44 (m, 1H), 7.32-7.21 (m, 2H), 7.01-6.90 (m, 2H), 3.85 (s, 3H), 3.64-3.54 (m, 1H), 3.11-3.09 (m, 1H), 2.54-2.39 (m, 3H), 1.94-1.90 (m, 1H), 1.65-1.61 (m, 3H), 1.45 (s, 9H)

### Step 3: N²-(4-((S)-3-aminopiperidin-1-yl)-5-(1-(difluoromethyl)-1H-pyrazol-4-yl)pyridin-2-yl)-6-(2-fluoro-6-methoxyphenyl)pyridin-2,5-diamine hydrochloride

tert-Butyl ((3S)-1-(2-((5-amino-6-(2-fluoro-6-methoxyphenyl)pyridin-2-yl)amino)-5-(1-(difluoromethyl)-1H-pyrazol-4-yl)pyridin-4-yl)piperidin-3-yl)carbamate (9.9 mg, 0.02 mmol) was dissolved in dichloromethane (1.0 ml) and then 4.0 M hydrogen chloride solution dissolved in 1,4-dioxane (0.02 mL, 0.01 mmol) was added dropwise thereto. Then, the mixed solution was stirred at room temperature for 2 hours. After completion of the reaction, the reaction mixture was concentrated and then dried to prepare the titled compound (4.6 mg). (Yield: 48.6%) 1H NMR (CD₃OD, 400 MHz) δ 8.47 (s, 1H), 8.05-8.02 (m, 2H), 7.83-7.74 (m, 1H), 7.64-7.59 (m, 2H), 7.33-7.31 (m, 1H), 7.11 (d, 1H), 7.01 (t, 1H), 6.92 (s, 1H), 3.92 (s, 3H), 3.77-3.75 (m, 1H), 3.50-3.44 (m, 2H), 2.97-2.84 (m, 2H), 2.21-2.19 (m, 1H), 1.83-1.71 (m, 2H), 1.61-1.59 (m, 1H) MS m/z=525.2 (M+H)+

### Example 152: 4-((S)-3-aminopiperidin-1-yl)-5-(1-(2,2-difluoroethyl)-1H-pyrazol-4-yl)-N-(6-(2-fluoro-6-methoxyphenyl)-5-nitropyridin-2-yl)pyridin-2-amine hydrochloride

The titled compound (9.4 mg) was prepared in accordance with the same procedures as in Example 52, using Intermediates A3-10 and C3-1. (Yield: 92.5%) 1H NMR (CD₃OD, 400 MHz) δ 8.59 (d, 1H), 8.13 (s, 1H), 8.05 (s, 1H), 7.89 (s, 1H), 7.58-7.52 (m, 1H), 7.35 (d, 1H), 7.05-6.95 (m, 3H), 6.42-6.92 (m, 1H), 4.73-4.64 (m, 2H), 3.83-3.80 (m, 4H), 3.58-3.55 (m, 1H), 3.42-3.39 (m, 1H), 2.95-2.88 (m, 2H), 2.22-2.20 (m, 1H), 1.88-1.85 (m, 1H), 1.78-1.75 (m, 1H), 1.65-1.58 (m, 1H). MS m/z=569.2 (M+H)+

### Example 153: 4-((S)-3-aminopiperidin-1-yl)-N-(6-(2-fluoro-6-methoxyphenyl)-5-nitropyridin-2-yl)-5-(1-(2,2,2-trifluoroethyl)-1H-pyrazol-4-yl)pyridin-2-amine hydrochloride

The titled compound (8.3 mg) was prepared in accordance with the same procedures as in Example 52, using Intermediates A3-4 and C3-1. (Yield: 79.4%) 1H NMR (CD₃OD, 400 MHz) δ 8.60 (d, 1H), 8.20-8.18 (m, 1H), 8.06 (s, 1H), 7.92 (s, 1H), 7.58-7.52 (m, 1H), 7.38-7.36 (m, 1H), 7.05-6.95 (m, 3H), 5.10-5.04 (m, 2H), 3.85-3.80 (m, 4H), 3.57-3.54 (m, 1H), 3.50-3.42 (m, 1H), 3.01-2.88 (m, 2H), 2.22-2.19 (m, 1H), 1.82-1.70 (m, 2H), 1.63-1.61 (m, 1H) MS m/z=587.2 (M+H)+

### Example 154: 4-((S)-3-aminopiperidin-1-yl)-N-(6-(2-fluoro-6-methoxyphenyl)-5-nitropyridin-2-yl)-5-(1-(trifluoromethyl)-1H-pyrazol-4-yl)pyridin-2-amine hydrochloride

The titled compound (3.9 mg) was prepared in accordance with the same procedures as in Example 52, using Intermediates A3-11 and C3-1. (Yield: 37.9%) 1H NMR (CD₃OD, 400 MHz) δ 8.62-8.59 (m, 2H), 8.20 (s, 1H), 8.14 (d, 1H), 7.58-7.52 (m, 1H), 7.37 (d, 1H), 7.09 (s, 1H), 7.02-6.95 (m, 2H), 3.80 (s, 4H), 3.56-3.50 (m, 1H), 3.40-3.38 (m, 1H), 3.01-2.93 (m, 2H), 2.22-2.20 (m, 1H), 1.85-1.72 (m, 2H), 1.70-1.60 (m, 1H) MS m/z=573.2 (M+H)+

### Example 155: 4-((S)-3-aminopiperidin-1-yl)-N-(6-(2-fluoro-6-methoxyphenyl)-5-nitropyridin-2-yl)-5-((tetrahydro-2H-pyran-4-yl)ethynyl)pyridin-2-amine hydrochloride

The titled compound (4.3 mg) was prepared in accordance with the same procedures as in Example 52, using Intermediates A7-1 and C3-1. (Yield: 43.8%) 1H NMR (CD₃OD, 400 MHz) δ 8.58 (d, 1H), 8.21 (s, 1H), 7.58-7.53 (m, 1H), 7.31 (d, 1H), 7.02-6.96 (m, 2H), 6.85 (s, 1H), 4.37-4.34 (m, 1H), 4.25-4.23 (m, 1H), 3.96-3.91 (m, 2H), 3.80 (s, 3H), 3.59-3.45 (m, 4H), 3.02-2.97 (m, 1H), 2.28-2.25 (m, 1H), 2.05-2.01 (m, 1H), 1.97-1.94 (m, 2H), 1.88-1.85 (m, 1H), 1.81-1.71 (m, 3H) MS m/z=547.2 (M+H)+

### Example 156: 4-((S)-3-aminopiperidin-1-yl)-N-(6-(2-fluoro-6-methoxyphenyl)-5-nitropyridin-2-yl)-5-((1-(trifluoromethyl)-1H-pyrazol-4-yl)ethynyl)pyridin-2-amine hydrochloride

The titled compound (9.9 mg) was prepared in accordance with the same procedures as in Example 52, using Intermediates A7-2 and C3-1. (Yield: 93.3%) 1H NMR (CD₃OD, 400 MHz) δ 8.61-8.56 (m, 2H), 8.34 (s, 1H), 8.06 (s, 1H), 7.57-7.52 (m, 1H), 7.36 (d, 1H), 7.02-6.95 (m, 3H), 4.39-4.35 (m, 1H), 4.11-4.08 (m, 1H), 3.80 (s, 3H), 3.52-3.51 (m, 1H), 3.43-3.37 (m, 1H), 2.27-2.24 (m, 1H), 2.06-2.01 (m, 1H), 1.90-1.72 (m, 3H) MS m/z=597.2 (M+H)+

### Example 157: (S)-N-(4-(3-aminopiperidin-1-yl)-5-(1-(trifluoromethyl)-1H-pyrazol-4-yl)pyridin-2-yl)-2-(2,4-difluoro-6-methoxyphenyl)pyrimidin-4-amine hydrochloride

The titled compound (7.8 mg) was prepared in accordance with the same procedures as in Example 52, using Intermediates A3-11 and C1-7. (Yield: 72.2%) 1H NMR (CD₃OD, 400 MHz) δ 8.78 (d, 1H), 8.66 (s, 1H), 8.25 (d, 2H), 7.62 (s, 1H), 7.27 (s, 1H), 7.02 (d, 1H), 6.91 (dd, 1H), 3.96 (s, 3H), 3.86-3.83 (m, 1H), 3.56-3.52 (m, 1H), 3.44-3.33 (m, 1H), 3.07-2.95 (m, 2H), 2.22-2.20 (m, 1H), 1.87-1.84 (m, 1H), 1.75-1.63 (m, 2H) MS m/z=547.2 (M+H)+

### Example 158: (S)-N-(4-(3-aminopiperidin-1-yl)-5-(1-(2,2-difluoroethyl)-1H-pyrazol-4-yl)pyridin-2-yl)-2-(2,4-difluoro-6-methoxyphenyl)pyrimidin-4-amine hydrochloride

The titled compound (15.1 mg) was prepared in accordance with the same procedures as in Example 52, using Intermediates A3-10 and C1-7. (Yield: 92.6%) 1H NMR (CD₃OD, 400 MHz) δ 8.79 (d, 1H), 8.19 (d, 2H), 7.9 (s, 1H), 7.52-7.50 (m, 1H), 7.23 (s, 1H), 7.01 (d, 1H), 6.89 (dd, 1H), 6.43-6.15 (m, 1H), 4.74-4.66 (m, 2H), 3.95 (s, 3H), 3.91-3.88 (m, 1H), 3.61-3.58 (m, 1H), 3.45-3.33 (m, 1H), 3.05-2.96 (m, 2H), 2.22-2.20 (m, 1H), 1.84-1.77 (m, 1H), 1.66-1.64 (m, 1H) MS m/z=543.2 (M+H)+

### Example 159: (S)-N-(4-(3-aminopiperidin-1-yl)-5-((1-(trifluoromethyl)-1H-pyrazol-4-yl)ethynyl)pyridin-2-yl)-2-(2,4-difluoro-6-methoxyphenyl)pyrimidin-4-amine hydrochloride

The titled compound (3.7 mg) was prepared in accordance with the same procedures as in Example 52, using Intermediates A7-2 and C1-7. (Yield: 85.7%) 1H NMR (CD₃OD, 400 MHz) δ 8.72 (d, 1H), 8.7 (s, 1H), 8.62 (s, 1H), 8.42 (s, 1H), 8.07 (s, 1H), 7.76 (s, 1H), 7.15 (s, 1H), 7.02 (d, 1H), 6.92 (dd, 1H), 4.31-4.28 (m, 1H), 4.09-4.06 (m, 1H), 3.95 (s, 3H), 3.53-3.52 (m, 1H), 3.40-3.33 (m, 2H), 2.26-2.23 (m, 1H), 2.11-2.03 (m, 1H), 1.91-1.88 (m, 1H), 1.76-1.67 (m, 1H) MS m/z=571.2 (M+H)+

### Example 160: 6-((5-(1-(2,2-difluoroethyl)-1H-pyrazol-4-yl)-4-((S)-3-hydroxypiperidin-1-yl)pyridin-2-yl)amino)-2-(2-fluoro-6-methoxyphenyl)nicotinamide

The titled compound (3.7 mg) was prepared in accordance with the same procedures as in Example 112, using Intermediates A2-10 and C2-1. (Yield: 35.6%) 1H NMR (CD₃OD, 400 MHz) δ 8.05 (d, 2H), 7.97 (d, 1H), 7.90 (s, 1H), 7.87 (d, 1H), 7.44-7.35 (m, 2H), 6.93-6.85 (m, 2H), 6.37-6.08 (m, 1H), 4.66-3.80 (m, 2H), 3.82 (s, 3H), 3.74-3.70 (m, 1H), 3.26-3.23 (m, 1H), 3.03-2.96 (m, 1H), 2.63-2.54 (m, 1H), 2.41 (t, 1H), 1.96-1.94 (m, 1H), 1.83-1.72 (m, 1H), 1.57-1.56 (m, 1H), 1.39-1.35 (m, 1H). MS m/z=567.9 (M+H)+

### Example 161: (S)-1-(2-((2-(2-fluoro-4-(hydroxymethyl)-6-methoxyphenyl)pyrimidin-4-yl)amino)-5-(1-(tetrahydro-2H-pyran-4-yl)-1H-pyrazol-4-yl)pyridin-4-yl)piperidin-3-ol

The titled compound (6.8 mg) was prepared in accordance with the same procedures as in Example 7, using Intermediates A2-1 and C5-1. (Yield: 33.9%) 1H NMR (CDCl₃, 400 MHz) δ 8.55 (d, 1H), 8.3 (s, 1H), 8.14 (s, 1H), 7.79 (s, 1H), 7.66 (s, 1H), 7.42 (s, 1H), 6.77-6.73 (m, 2H), 4.7 (s, 2H), 4.42-4.38 (m, 1H), 4.15-4.12 (m, 2H), 3.77 (s, 3H), 3.75-3.74 (m, 1H), 3.60-3.54 (m, 2H), 3.21-3.18 (m, 1H), 2.99-2.96 (m, 1H), 2.63-2.59 (m, 2H), 2.14-2.05 (m, 2H), 1.91-1.89 (m, 1H), 1.70-1.49 (m, 3H), 1.46-1.41 (m, 2H) MS m/z=576.2 (M+H)+

### Example 162: (S)-N-(4-(3-aminopiperidin-1-yl)-5-(1-(2-morpholinoethyl)-1H-pyrazol-4-yl)pyridin-2-yl)-2-(2-fluoro-6-methoxyphenyl)pyrimidin-4-amine hydrochloride

The titled compound (12.0 mg) was prepared in accordance with the same procedures as in Example 52, using Intermediates A3-12 and C1-1. (Yield: 73.1%) 1H NMR (CD₃OD, 400 MHz) δ 8.60 (d, 1H), 8.22 (s, 1H), 8.19 (s, 1H), 7.57 (s, 1H), 7.65-7.50 (m, 2H), 7.34 (s, 1H), 7.05 (d, 1H), 6.92 (t, 1H), 4.72 (t, 2H), 3.96 (t, 4H), 3.88 (s, 3H), 3.80-3.70 (m, 1H), 3.70-3.58 (m, 2H), 3.52-3.20 (m, 6H), 2.90-2.80 (m, 2H), 2.25-2.00 (m, 3H), 1.94-1.80 (m, 1H) MS m/z=575.0 (M+H)+

### Example 163: (S)-N-(4-(3-aminopiperidin-1-yl)-5-(1-(2-(pyrrolidin-1-yl)ethyl)-1H-pyrazol-4-yl)pyridin-2-yl)-2-(2-fluoro-6-methoxyphenyl)pyrimidin-4-amine hydrochloride

The titled compound (11.4 mg) was prepared in accordance with the same procedures as in Example 52, using Intermediates A3-13 and C1-1. (Yield: 85.7%) 1H NMR (CD₃OD, 400 MHz) δ 8.52 (d, 1H), 8.28-8.15 (m, 2H), 7.97 (s, 1H), 7.60 (s, 1H), 7.58-7.42 (m, 2H), 7.02 (d, 1H), 6.90 (t, 1H), 4.70 (t, 2H), 3.86 (s, 3H), 3.83 (t, 2H), 3.72-3.60 (m, 1H), 3.60-3.30 (m, 4H), 3.30-3.20 (m, 2H), 2.85-2.60 (m, 2H), 2.25-2.10 (m, 4H), 2.10-2.00 (m, 2H), 1.90-1.74 (m, 1H), 1.65-1.50 (m, 1H) MS m/z=558.0 (M+H)+

### Example 164: (S)-N-(4-(3-aminopiperidin-1-yl)-5-((1-(difluoromethyl)-1H-pyrazol-4-yl)ethynyl)pyridin-2-yl)-2-(2-fluoro-6-methoxyphenyl)pyrimidin-4-amine hydrochloride

The titled compound (16.1 mg) was prepared in accordance with the same procedures as in Example 52, using Intermediates A7-3 and C1-1. (Yield: 48.4%) 1H NMR (CD₃OD, 400 MHz) δ 8.77 (d, 1H), 8.49 (s, 1H), 8.43 (s, 1H), 7.96-7.72 (m, 1H), 7.71-7.42 (m, 1H), 7.23 (s, 1H), 7.18 (d, 1H), 7.07 (t, 1H), 4.42-4.39 (m, 1H), 4.24-4.21 (m, 1H), 3.98 (s, 3H), 3.53-3.51 (m, 1H), 3.47-3.40 (m, 2H), 2.28-2.26 (m, 1H), 2.24-2.05 (m, 11H), 2.04-1.82 (m, 2H), MS m/z=535.2 (M+H)+

### Example 165: (S)-N-(4-(3-aminopiperidin-1-yl)-5-((1-(2,2-difluoroethyl)-1H-pyrazol-4-yl)ethynyl)pyridin-2-yl)-2-(2-fluoro-6-methoxyphenyl)pyrimidin-4-amine hydrochloride

The titled compound (19.6 mg) was prepared in accordance with the same procedures as in Example 52, using Intermediates A7-4 and C1-1. (Yield: 57.7%) 1H NMR (CD₃OD, 400 MHz) δ 8.78 (d, 1H), 8.39 (s, 1H), 8.11 (s, 1H), 7.81 (s, 1H), 7.74-7.69 (m, 2H), 7.21 (s, 1H), 7.18 (d, 1H), 7.07 (t, 1H), 6.37-6.10 (m, 1H), 4.68-4.60 (m, 2H), 4.43-4.40 (m, 1H), 4.31-4.26 (m, 1H), 3.98 (s, 3H), 3.53-3.51 (m, 1H), 3.48-3.43 (m, 2H), 2.28-2.26 (m, 1H), 2.25-2.12 (m, 1H), 2.04-1.82 (m, 2H) MS m/z=549.2 (M+H)+

### Example 166: (S)-N-(4-(3-aminopiperidin-1-yl)-5-((1-(2,2,2-trifluoroethyl)-1H-pyrazol-4-yl)ethynyl)pyridin-2-yl)-2-(2-fluoro-6-methoxyphenyl)pyrimidin-4-amine hydrochloride

The titled compound (13.8 mg) was prepared in accordance with the same procedures as in Example 52, using Intermediates A7-5 and C1-1. (Yield: 39.5%) 1H NMR (CD₃OD, 400 MHz) δ 8.76 (d, 1H), 8.4 (s, 1H), 8.18 (s, 1H), 7.83 (s, 1H), 7.73-7.67 (m, 2H), 7.2 (s, 1H), 7.17 (d, 1H), 7.06 (t, 1H), 5.06 (q, 2H), 4.39-4.36 (m, 1H), 4.23-4.20 (m, 1H), 3.97 (s, 3H), 3.53-3.51 (m, 1H), 3.50-3.38 (m, 2H), 2.24-2.24 (m, 1H), 2.07-2.04 (m, 1H), 1.92-1.81 (m, 2H), MS m/z=567.2 (M+H)+

### Example 167: (S)-3-(4-(4-(3-aminopiperidin-1-yl)-6-((2-(2-fluoro-6-methoxyphenyl)pyrimidin-4-yl)amino)pyridin-3-yl)-1H-pyrazol-1-yl)propanamide hydrochloride

The titled compound (1.3 mg) was prepared in accordance with the same procedures as in Example 52, using Intermediates A3-14 and C1-1. (Yield: 40.0%) 1H NMR (CD₃OD, 400 MHz) δ 8.60 (d, 1H), 8.22 (s, 1H), 8.19 (s, 1H), 7.57 (s, 1H), 7.65-7.50 (m, 2H), 7.34 (s, 1H), 7.05 (d, 1H), 6.92 (t, 1H), 4.72 (t, 2H), 3.96 (t, 4H), 3.88 (s, 3H), 3.80-3.70 (m, 1H), 3.70-3.58 (m, 2H), 3.52-3.20 (m, 6H), 2.90-2.80 (m, 2H), 2.25-2.00 (m, 3H), 1.94-1.80 (m, 1H) MS m/z=531.9 (M+H)+

### Example 168: (S)-N-(4-(3-aminopiperidin-1-yl)-5-(1-(azetidin-3-yl)-1H-pyrazol-4-yl)pyridin-2-yl)-2-(2-fluoro-6-methoxyphenyl)pyrimidin-4-amine hydrochloride

The titled compound (1.3 mg) was prepared in accordance with the same procedures as in Example 52, using Intermediates A3-15 and C1-1. (Yield: 11.8%) 1H NMR (CD₃OD, 400 MHz) δ 8.47 (d, 1H), 8.43 (s, 1H), 7.84 (d, 1H), 7.67 (s, 1H), 7.61 (d, 1H), 7.47 (q, 1H), 6.99 (d, 1H), 6.88 (t, 1H), 6.75 (d, 1H), 5.55-5.44 (m, 1H), 4.60-4.50 (m, 2H), 4.50-4.40 (m, 2H), 3.84 (s, 3H), 3.36 (d, 1H), 3.35-3.15 (m, 2H), 2.80-2.62 (m, 2H), 2.10-2.00 (m, 2H), 1.80-1.66 (m, 1H), 1.66-1.46 (m, 1H) MS m/z=515.9 (M+H)+

### Example 169: N²-(4-((S)-3-aminopiperidin-1-yl)-5-(1-(2,2-difluoroethyl)-1H-pyrazol-4-yl)pyridin-2-yl)-6-(2-fluoro-6-methoxyphenyl)pyridin-2,5-diamine hydrochloride

The titled compound (2.1 mg) was prepared in accordance with the same procedures as in Example 151, using Intermediates A3-10 and C3-1. (Yield: 48.1%) 1H NMR (CD₃OD, 400 MHz) δ 8.09 (s, 1H), 7.95 (s, 1H), 7.86 (s, 1H), 7.67 (d, 1H), 7.61-7.55 (m, 1H), 7.19 (d, 1H), 7.09 (d, 1H), 6.98 (t, 1H), 6.80 (s, 1H), 6.40-6.13 (m, 1H), 4.71-4.62 (m, 2H), 3.90 (s, 3H), 3.74-3.71 (m, 1H), 3.51-3.37 (m, 2H), 2.87-2.80 (m, 2H), 2.22-2.19 (m, 1H), 1.92-1.74 (m, 2H), 1.63-1.53 (m, 1H) MS m/z=538.9 (M+H)+

### Example 170: N²-(4-((S)-3-aminopiperidin-1-yl)-5-(1-(2,2,2-trifluoroethyl)-1H-pyrazol-4-yl)pyridin-2-yl)-6-(2-fluoro-6-methoxyphenyl)pyridin-2,5-diamine hydrochloride

The titled compound (6.1 mg) was prepared in accordance with the same procedures as in Example 151, using Intermediates A3-4 and C3-1. (Yield: 58.4%) 1H NMR (CD₃OD, 400 MHz) δ 8.16 (s, 1H), 7.97 (s, 1H), 7.90 (s, 1H), 7.71-7.66 (m, 1H), 7.59-7.55 (m, 1H), 7.24-7.18 (m, 1H), 7.09 (d, 1H), 6.99 (t, 1H), 6.84-6.80 (m, 1H), 5.08-5.02 (m, 2H), 3.90 (s, 3H), 3.76-3.73 (m, 1H), 3.50-3.40 (m, 2H), 2.91-2.79 (m, 2H), 2.22-2.19 (m, 1H), 1.81-1.69 (m, 2H), 1.64-1.54 (m, 1H) MS m/z=556.9 (M+H)+

### Example 171: (S)-1-(2-((2-(2,4-difluoro-6-methoxyphenyl)pyrimidin-4-yl)amino)-5-(1-(trifluoromethyl)-1H-pyrazol-4-yl)pyridin-4-yl)piperidin-3-ol

The titled compound (5.3 mg) was prepared in accordance with the same procedures as in Example 7, using Intermediates A2-11 and C1-7. (Yield: 16.8%) 1H NMR (CDCl₃, 400 MHz) δ 8.60 (d, 1H), 8.58 (d, 1H), 8.16 (s, 1H), 7.69 (s, 1H), 7.44-7.43 (m, 1H), 7.39 (s, 1H), 6.60-6.55 (m, 2H), 3.86-3.84 (m, 1H), 3.82 (s, 3H), 3.24-3.23 (m, 1H), 3.20-2.97 (m, 1H), 2.73-2.67 (m, 2H), 1.96-1.94 (m, 1H), 1.79-1.77 (m, 1H), 1.54-1.47 (m, 2H) MS m/z=547.9 (M+H)+

### Example 172: (S)-1-(2-((2-(2,4-difluoro-6-methoxyphenyl)pyrimidin-4-yl)amino)-5-(1-(2,2-difluoroethyl)-1H-pyrazol-4-yl)pyridin-4-yl)piperidin-3-ol

The titled compound (3.9 mg) was prepared in accordance with the same procedures as in Example 7, using Intermediates A2-10 and C1-7. (Yield: 12.4%) 1H NMR (CDCl₃, 400 MHz) δ 8.58 (d, 1H), 8.11 (s, 1H), 7.84 (d, 2H), 7.81 (s, 1H), 7.47 (s, 1H), 7.31-7.28 (m, 1H), 6.60-6.59 (m, 2H), 6.32-6.16 (m, 1H), 4.57-4.49 (m, 2H), 3.84-3.83 (m, 1H), 3.82 (s, 3H), 3.25-3.22 (m, 1H), 2.92-2.71 (m, 1H), 2.68-2.66 (m, 2H), 1.78-1.76 (m, 1H), 1.68-1.56 (m, 1H), 1.53-1.48 (m, 2H) MS m/z=543.9 (M+H)+

### Example 173: N²-(4-((S)-3-aminopiperidin-1-yl)-5-(1-(trifluoromethyl)-1H-pyrazol-4-yl)pyridin-2-yl)-6-(2-fluoro-6-methoxyphenyl)pyridin-2,5-diamine hydrochloride

The titled compound (2.8 mg) was prepared in accordance with the same procedures as in Example 151, using Intermediates A3-11 and C3-1. (Yield: 38.6%) 1H NMR (CD₃OD, 400 MHz) δ 8.56 (s, 1H), 8.19 (s, 1H), 8.03 (s, 1H), 7.72-7.56 (m, 2H), 7.25-7.17 (m, 1H), 7.09 (d, 1H), 6.98 (t, 1H), 6.85-6.77 (m, 1H), 3.90 (s, 3H), 3.74-3.71 (m, 1H), 3.43-3.38 (m, 2H), 2.93-2.85 (m, 2H), 2.22-2.18 (m, 1H), 1.85-1.83 (m, 1H), 1.73-1.70 (m, 1H), 1.63-1.57 (m, 1H) MS m/z=542.9 (M+H)+

### Example 174: (S)-1-(2-((2-(2,4-difluoro-6-methoxyphenyl)pyrimidin-4-yl)amino)-5-(1-(2,2,2-trifluoroethyl)-1H-pyrazol-4-yl)pyridin-4-yl)piperidin-3-ol

The titled compound (2.0 mg) was prepared in accordance with the same procedures as in Example 7, using Intermediates A2-6 and C1-7. (Yield: 6.2%) 1H NMR (CDCl₃, 400 MHz) δ 8.58 (d, 1H), 8.12 (s, 1H), 7.87 (d, 1H), 7.65 (s, 1H), 7.52-7.47 (m, 1H), 7.31-7.28 (m, 1H), 6.60-6.55 (m, 2H), 4.81 (q, 2H), 3.82 (s, 3H), 3.81-3.80 (m, 1H), 3.26-3.22 MS m/z=561.9 (M+H)+

### Example 175: (S)-5-(3-aminopiperidin-1-yl)-N-(2-(2-fluoro-6-methoxyphenyl)pyrimidin-4-yl)-6-(1-(2,2,2-trifluoroethyl)-1H-pyrazol-4-yl)pyridazin-3-amine hydrochloride

The titled compound (8.1 mg) was prepared in accordance with the same procedures as in Example 52, using Intermediates A10-1 and C1-1. (Yield: 48.1%) 1H NMR (CD₃OD, 400 MHz) δ 8.52 (d, 1H), 8.37 (s, 1H), 8.17 (s, 1H), 8.09 (s, 1H), 7.54-7.42 (m, 2H), 7.00 (d, 1H), 6.89 (t, 1H), 5.08 (q, 2H), 3.84 (s, 3H), 3.20 (d, 1H), 3.00-2.86 (m, 1H), 2.70-2.56 (m, 1H), 2.03 (t, 1H), 2.10-2.00 (m, 2H), 1.78-1.68 (m, 1H), 1.30-1.16 (m, 1H) MS m/z=544.9 (M+H)+

### Example 176: (S)-(4-(4-(3-aminopiperidin-1-yl)-6-((2-(2-fluoro-6-methoxyphenyl)pyrimidin-4-yl)amino)pyridin-3-yl)phenyl)(4-methylpiperazin-1-yl)methanone hydrochloride

The titled compound (10.5 mg) was prepared in accordance with the same procedures as in Example 52, using Intermediates A3-20 and C1-1. (Yield: 85.4%) 1H NMR (CD₃OD, 400 MHz) δ 8.53 (d, 1H), 8.08 (s, 1H), 7.74 (dd, 2H), 7.68-7.58 (m, 3H), 7.55 (s, 1H), 7.50 (q, 1H), 7.01 (d, 1H), 6.89 (t, 1H), 3.92-3.80 (m, 1H), 3.85 (s, 3H), 3.50-3.24 (m, 4H), 3.24-3.10 (m, 2H), 2.94 (s, 3H), 2.80-2.60 (m, 2H), 2.15-2.00 (m, 4H), 1.80-1.70 (m, 1H), 1.70-1.56 (m, 2H), 1.56-1.44 (m, 1H) MS m/z=597.0 (M+H)+

### Example 177: (3S)-1-(2-((5-amino-6-(2-fluoro-6-methoxyphenyl)pyridin-2-yl)amino)-5-(1-(tetrahydro-2H-pyran-4-yl)-1H-pyrazol-4-yl)pyridin-4-yl)piperidin-3-ol

The titled compound (7.3 mg) was prepared in accordance with the same procedures as in Example 150, using Intermediates A2-1 and C3-1. (Yield: 51.3%) 1H NMR (CD₃OD, 400 MHz) δ 8.04 (s, 1H), 7.92 (s, 1H), 7.79 (s, 1H), 7.49-7.43 (m, 1H), 7.33-7.29 (m, 1H), 7.25-7.22 (m, 1H), 6.99 (d, 1H), 6.89 (t, 1H), 4.49-4.45 (m, 1H), 4.13-4.09 (m, 2H), 3.85 (s, 3H), 3.74-3.68 (m, 1H), 3.64-3.58 (m, 2H), 3.28-3.25 (m, 1H), 3.03-3.01 (m, 1H), 2.62-2.59 (m, 1H), 2.46-2.40 (m, 1H), 2.15-2.05 (m, 4H), 1.98-1.92 (m, 1H), 1.76-1.73 (m, 1H), 1.57-1.55 (m, 1H), 1.37-1.35 (m, 1H) MS m/z=559.9 (M+H)+

### Example 178: (3S)-1-(2-((5-amino-6-(2-fluoro-6-methoxyphenyl)pyridin-2-yl)amino)-5-(1-(trifluoromethyl)-1H-pyrazol-4-yl)pyridin-4-yl)piperidin-3-ol

The titled compound (2.8 mg) was prepared in accordance with the same procedures as in Example 150, using Intermediates A2-11 and C3-1. (Yield: 35.2%) 1H NMR (CD₃OD, 400 MHz) δ 8.48 (s, 1H), 8.20 (s, 1H), 8.00 (s, 1H), 7.49-7.41 (m, 2H), 7.31-7.26 (m, 2H), 6.99 (d, 1H), 6.89 (t, 1H), 3.86 (s, 3H), 3.82 (s, 1H), 3.23-3.20 (m, 1H), 2.99-2.98 (m, 1H), 2.65-2.62 (m, 1H), 2.51-2.46 (m, 1H), 1.97-1.92 (m, 1H), 1.78-1.75 (m, 1H), 1.56-1.54 (m, 1H), 1.38-1.35 (m, 1H) MS m/z=543.9 (M+H)+

### Example 179: (3S)-1-(2-((5-amino-6-(2-fluoro-6-methoxyphenyl)pyridin-2-yl)amino)-5-(1-(2,2-difluoroethyl)-1H-pyrazol-4-yl)pyridin-4-yl)piperidin-3-ol

The titled compound (6.6 mg) was prepared in accordance with the same procedures as in Example 150, using Intermediates A2-10 and C3-1. (Yield: 32.4%) 1H NMR (CD₃OD, 400 MHz) δ 8.02 (s, 1H), 7.92 (s, 1H), 7.86 (s, 1H), 7.49-7.43 (m, 1H), 7.31-7.29 (m, 2H), 7.25-7.22 (m, 1H), 6.99 (d, 1H), 6.89 (t, 1H), 6.36-6.08 (m, 1H), 4.66-4.57 (m, 2H), 3.85 (s, 3H), 3.76-3.70 (m, 1H), 3.28-3.25 (m, 1H), 3.06-3.02 (m, 1H), 2.63-2.56 (m, 1H), 2.46-2.41 (m, 1H), 1.98-1.92 (m, 1H), 1.75-1.72 (m, 1H), 1.58-1.55 (m, 1H), 1.37-1.34 (m, 1H). MS m/z=539.9 (M+H)+

### Example 180: (3S)-1-(2-((5-amino-6-(2-fluoro-6-methoxyphenyl)pyridin-2-yl)amino)-5-(1-(2,2,2-trifluoroethyl)-1H-pyrazol-4-yl)pyridin-4-yl)piperidin-3-ol

The titled compound (6.9 mg) was prepared in accordance with the same procedures as in Example 150, using Intermediates A2-6 and C3-1. (Yield: 39.9%) 1H NMR (CD₃OD, 400 MHz) δ 8.08 (s, 1H), 7.93 (s, 1H), 7.90 (s, 1H), 7.49-7.43 (m, 1H), 7.33-7.29 (m, 2H), 7.26-7.22 (m, 1H), 6.99 (d, 1H), 6.89 (t, 1H), 5.02-4.98 (m, 2H), 3.86 (s, 3H), 3.75-3.71 (m, 1H), 3.28-3.26 (m, 1H), 3.03-3.01 (m, 1H), 2.62-2.55 (m, 1H), 2.48-2.42 (m, 1H), 1.97-1.92 (m, 1H), 1.74-1.71 (m, 1H), 1.64-1.47 (m, 1H), 1.36-1.34 (m, 1H). MS m/z=557.9 (M+H)+

### Example 181: N²-(4-((S)-3-aminopiperidin-1-yl)-5-(1-(tetrahydro-2H-pyran-4-yl)-1H-pyrazol-4-yl)pyridin-2-yl)-6-(2-fluoro-6-methoxyphenyl)pyridin-2,5-diamine hydrochloride

The titled compound (7.5 mg) was prepared in accordance with the same procedures as in Example 151, using Intermediates A3-1 and C3-1. (Yield: 76.0%) 1H NMR (CD₃OD, 400 MHz) δ 8.09 (s, 1H), 7.96 (s, 1H), 7.88-7.84 (m, 1H), 7.81 (s, 1H), 7.63-7.61 (m, 1H), 7.32-7.30 (m, 1H), 7.12 (d, 1H), 7.02 (t, 1H), 6.92-6.89 (m, 1H), 4.52-4.49 (m, 1H), 4.13-4.09 (m, 2H), 3.92 (s, 3H), 3.82-3.79 (m, 1H), 3.64-3.58 (m, 2H), 3.54-3.41 (m, 2H), 2.97-2.92 (m, 1H), 2.87-2.81 (m, 1H), 2.22-2.19 (m, 1H), 2.15-2.03 (m, 4H), 1.81-1.76 (m, 1H), 1.74-1.71 (m, 1H), 1.65-1.59 (m, 1H). MS m/z=559.3 (M+H)+

### Example 182: (S)-1-(2-((2-(2,4-difluoro-6-methoxyphenyl)pyrimidin-4-yl)amino)-5-(1-(difluoromethyl)-1H-pyrazol-4-yl)pyridin-4-yl)piperidin-3-ol

The titled compound (4.9 mg) was prepared in accordance with the same procedures as in Example 7, using Intermediates A2-8 and C1-7. (Yield: 15.2%) 1H NMR (CDCl₃, 400 MHz) δ 8.59 (d, 1H), 8.15 (d, 1H), 7.97 (s, 1H), 7.75 (s, 1H), 7.46-7.44 (m, 1H), 7.41-7.10 (m, 2H), 6.59-6.54 (m, 2H), 3.86-3.84 (m, 1H), 3.82 (s, 3H), 3.25-3.21 (m, 1H), 2.98-2.82 (m, 1H), 2.70-2.65 (m, 2H), 2.06-1.92 (m, 1H), 1.77-1.76 (m, 1H), 1.75-1.74 (m, 1H), 1.56-1.50 (m, 2H), MS m/z=529.9 (M+H)+

### Example 183: (3S)-1-(2-((6-(2,4-difluoro-6-methoxyphenyl)-5-nitropyridin-2-yl)amino)-5-(1-(difluoromethyl)-1H-pyrazol-4-yl)pyridin-4-yl)piperidin-3-ol

The titled compound (8.0 mg) was prepared in accordance with the same procedures as in Example 7, using Intermediates A2-8 and C3-2. (Yield: 39.2%) 1H NMR (CDCl₃, 400 MHz) δ 8.41 (d, 1H), 8.15 (d, 2H), 7.97 (s, 1H), 7.92 (s, 1H), 7.53-7.48 (m, 2H), 7.41-7.10 (m, 1H), 6.66 (d, 1H), 6.55 (dd, 1H), 3.86-3.84 (m, 1H), 3.75 (s, 3H), 3.21-2.97 (m, 1H), 2.69-2.67 (m, 1H), 2.66-2.62 (m, 2H), 1.93-1.79 (m, 1H), 1.78-1.76 (m, 1H), 1.56-1.51 (m, 2H), MS m/z=573.9 (M+H)+

### Example 184: (3S)-1-(2-((5-amino-6-(2,4-difluoro-6-methoxyphenyl)pyridin-2-yl)amino)-5-(1-(difluoromethyl)-1H-pyrazol-4-yl)pyridin-4-yl)piperidin-3-ol

The titled compound (1.8 mg) was prepared in accordance with the same procedures as in Step 2 of Example 150, using the compound prepared in Example 183. (Yield: 30.1%) 1H NMR (CD₃OD, 400 MHz) δ 8.35 (s, 1H), 8.06 (s, 1H), 7.97 (s, 1H), 7.66-7.38 (m, 1H), 7.36 (s, 1H), 7.29-7.26 (m, 2H), 6.82 (d, 1H), 6.74 (dd, 1H), 3.85 (s, 3H), 3.73-3.71 (m, 1H), 3.32-3.26 (m, 1H), 3.23-3.00 (m, 1H), 2.58-2.56 (m, 1H), 2.43-2.42 (m, 1H), 1.98-1.96 (m, 1H), 1.74-1.72 (m, 1H), 1.57-1.37 (m, 1H), 1.34-1.31 (m, 1H), MS m/z=543.9 (M+H)+

### Example 185: methyl (S)-4-(4-((4-(3-aminopiperidin-1-yl)-5-(1-(difluoromethyl)-1H-pyrazol-4-yl)pyridin-2-yl)amino)pyrimidin-2-yl)-3-fluoro-5-methoxybenzoate hydrochloride

The titled compound (9.8 mg) was prepared in accordance with the same procedures as in Example 52, using Intermediates A3-2 and C1-10. (Yield: 51.0%) 1H NMR (CD₃OD, 400 MHz) δ 8.82 (d, 1H), 8.52 (s, 1H), 8.19 (s, 1H), 8.07 (s, 1H), 7.76-7.46 (m, 4H), 7.21 (s, 1H), 4 (s, 3H), 3.99 (s, 3H), 3.87-3.85 (m, 1H), 3.58-3.54 (m, 1H), 3.50-3.43 (m, 1H), 3.06-2.97 (m, 2H), 2.22-2.20 (m, 1H), 1.76-1.73 (m, 1H), 1.65-1.62 (m, 2H), MS m/z=568.9 (M+H)+

### Example 186: (S)-4-(4-((4-(3-aminopiperidin-1-yl)-5-(1-(difluoromethyl)-1H-pyrazol-4-yl)pyridin-2-yl)amino)pyrimidin-2-yl)-3-fluoro-5-methoxy-N-methylbenzamide hydrochloride

### Step 1: methyl (S)-4-(4-((4-(3-((tert-butoxycarbonyl)amino)piperidin-1-yl)-5-(1-(difluoromethyl)-1H-pyrazol-4-yl)pyridin-2-yl)amino)pyrimidin-2-yl)-3-fluoro-5-m ethoxybenzoate

The titled compound (151.5 mg) was prepared in accordance with the same procedures as in Step 1 of Example 52, using Intermediates A3-2 and C1-10. (Yield: 32.3%) 1H NMR (CDCl₃, 400 MHz) δ 8.61 (d, 1H), 8.13 (s, 2H), 7.95 (s, 1H), 7.53 (s, 1H), 7.42 (s, 2H), 7.28-7.11 (m, 2H), 4.51-4.50 (m, 1H), 4.15 (s, 3H), 3.98 (s, 3H), 3.72-3.71 (m, 1H), 3.43-3.40 (m, 1H), 3.05-3.02 (m, 1H), 2.59-2.47 (m, 2H), 1.95-1.91 (m, 1H), 1.62-1.59 (m, 2H), 1.46 (s, 9H)

### Step 2: (S)-4-(4-((4-(3-((tert-butoxycarbonyl)amino)piperidin-1-yl)-5-(1-(difluoromethyl)-1H-pyrazol-4-yl)pyridin-2-yl)amino)pyrimidin-2-yl)-3-fluoro-5-methoxybenzoic acid

Methyl (S)-4-(4-((4-(3-((tert-butoxycarbonyl)amino)piperidin-1-yl)-5-(1-(difluoromethyl)-1H-pyrazol-4-yl)pyridin-2-yl)amino)pyrimidin-2-yl)-3-fluoro-5-methoxybenzoate (130.0 mg, 0.19 mmol) was dissolved in methanol (2.0 ml) and then 1.0 N sodium hydroxide solution (1.0 mL) was added dropwise thereto. Then, the mixed solution was stirred at 60 °C overnight. After completion of the reaction, the reaction mixture was cooled to room temperature and then neutralized with 1.0 N hydrochloric acid solution. The aqueous layer was extracted with dichloromethane. The extracted organic layer was dried over anhydrous magnesium sulfate and then filtered. The filtrate was concentrated and then dried to prepare the titled compound (98.0 mg), without further purification. (Yield: 77.0%) 1H NMR (CD₃OD, 400 MHz) δ 8.76 (d, 1H), 8.37 (s, 1H), 8.05 (s, 1H), 7.95 (s, 1H), 7.65-7.41 (m, 2H), 7.22 (s, 1H), 6.59 (s, 1H), 3.93 (s, 3H), 3.57-3.56 (m, 1H), 3.54-3.50 (m, 2H), 3.02-2.96 (m, 1H), 2.86-2.80 (m, 1H), 1.62-1.61 (m, 1H), 1.51-1.48 (m, 1H), 1.45-1.44 (m, 1H), 1.27-1.25 (m, 1H), 1.24 (s, 9H)

### Step 3: tert-butyl (S)-(1-(5-(1-(difluoromethyl)-1H-pyrazol-4-yl)-2-((2-(2-fluoro-6-methoxy-4-(methylcarbamoyl)phenyl)pyrimidin-4-yl)amino)pyridin-4-yl)piperidin-3-yl)carbamate

(S)-4-(4-((4-(3-((tert-Butoxycarbonyl)amino)piperidin-1-yl)-5-(1-(difluoromethyl)-1H-pyrazol-4-yl)pyridin-2-yl)amino)pyrimidin-2-yl)-3-fluoro-5-methoxybenzoic acid (20.0 mg, 0.031 mmol) was dissolved in anhydrous dimethylformamide (1.0 mL) and then 1-[bis(dimethylamino)methylene]-1H-1,2,3-triazolo[4,5-b]pyridinium-3-ox ide hexafluorophosphate (HATU, 23.2 mg, 0.061 mmol) and diisopropylethylamine (16 uL, 0.092 mmol) were added dropwise thereto. The reaction mixture was stirred at room temperature for 15 minutes and then 2.0M methylamine dissolved in tetrahydrofuran (30.5 uL, 0.061 mmol) was added dropwise thereto. The reaction mixture was stirred at 80 °C for 4 hours. Water was added to the reaction mixture and then the aqueous layer was extracted with ethyl acetate. The extracted organic layer was dried over anhydrous magnesium sulfate and then filtered. The filtrate was concentrated and then purified by silica gel column chromatography to prepare the titled compound (10.6 mg). (Yield: 51.9%) 1H NMR (CD₃OD, 400 MHz) δ 8.6 (d, 1H), 8.11 (s, 2H), 8 (s, 1H), 7.54 (s, 1H), 7.35-7.02 (m, 2H), 4.82-4.77 (m, 1H), 3.9 (s, 3H), 3.85-3.82 (m, 1H), 3.26-3.25 (m, 1H), 3.21-3.16 (m, 1H), 3.06 (s, 3H), 2.98-2.71 (m, 2H), 2.14-2.11 (m, 1H), 2.10-2.09 (m, 1H), 2.07-2.06 (m, 1H), 1.47 (s, 9H)

### Step 4: (S)-4-(4-((4-(3-aminopiperidin-1-yl)-5-(1-(difluoromethyl)-1H-pyrazol-4-yl)pyridin-2-yl)amino)pyrimidin-2-yl)-3-fluoro-5-methoxy-N-methylbenzamide hydrochloride

tert-Butyl (S)-(1-(5-(1-(difluoromethyl)-1H-pyrazol-4-yl)-2-((2-(2-fluoro-6-meth oxy-4-(methylcarbamoyl)phenyl)pyrimidin-4-yl)amino)pyridin-4-yl)piperidin-3-yl)c arbamate (10.6 mg, 0.016 mmol) was dissolved in dichloromethane (2.0 ml) and then 4.0 M hydrochloric acid solution dissolved in 1,4-dioxane (0.2 ml, 27.9 mmol) was added dropwise thereto. Then, the mixed solution was stirred at room temperature for 4 hours. After completion of the reaction, the reaction mixture was concentrated and the resulting solid was dried to prepare the titled compound (7.3 mg). (Yield: 71.2%) 1H NMR (CD₃OD, 400 MHz) 8.82 (d, 1H), 8.54 (s, 1H), 8.21 (s, 1H), 8.08 (s, 1H), 7.77-7.46 (m, 3H), 7.43 (s, 1H), 7.28 (s, 1H), 4.01 (s, 3H), 3.89-3.87 (m, 1H), 3.59-3.56 (m, 1H), 3.44-3.33 (m, 1H), 3.08-3.02 (m, 2H), 2.98 (s, 3H), 2.22-2.20 (m, 1H), 1.75-1.72 (m, 1H), 1.69-1.64 (m, 2H) MS m/z=568.9 (M+H)+

### Example 187: (S)-4-(4-((4-(3-aminopiperidin-1-yl)-5-(1-(difluoromethyl)-1H-pyrazol-4-yl)pyridin-2-yl)amino)pyrimidin-2-yl)-3-fluoro-5-methoxy-N,N-dimethylbenzamide hydrochloride

The titled compound (9.0 mg) was prepared in accordance with the same procedures as in Step 3 of Example 186, changing the amine reagent to 2.0M dimethylamine dissolved in tetrahydrofuran. (Yield: 49.1%) 1H NMR (CD₃OD, 400 MHz) δ 8.85-8.83 (m, 1H), 8.55 (s, 1H), 8.23 (s, 1H), 8.09 (s, 1H), 7.77-7.48 (m, 2H), 7.33 (s, 1H), 7.19 (s, 1H), 7.1 (d, 1H), 4.03 (s, 3H), 3.93-3.90 (m, 1H), 3.63-3.59 (m, 1H), 3.45-3.33 (m, 1H), 3.16 (s, 3H), 3.13 (s, 3H), 3.08-2.98 (m, 2H), 2.23-2.20 (m, 1H), 1.84-1.78 (m, 1H), 1.76-1.65 (m, 2H) MS m/z=582.0 (M+H)+

### Example 188: (S)-1-(2-((2-(4-((3-hydroxyazetidin-1-yl)methyl)thiophen-2-yl)pyrimidin-4-yl)amino)-5-(1-(tetrahydro-2H-pyran-4-yl)-1H-pyrazol-4-yl)pyridin-4-yl)piperidin-3-ol

The titled compound (7.4 mg) was prepared in accordance with the same procedures as in Example 7, using Intermediates A2-1 and C6-2. (Yield: 23.6%) 1H NMR (CD₃OD, 400 MHz) δ 8.31 (d, 1H), 8.13 (d, 1H), 7.91 (s, 1H), 7.87 (s, 1H), 7.77 (s, 1H), 7.46 (s, 1H), 7.2 (d, 1H), 4.48-4.42 (m, 1H), 4.40-4.39 (m, 1H), 4.11-4.08 (m, 2H), 3.84-3.72 (m, 3H), 3.64-3.60 (m, 2H), 3.58-3.48 (m, 1H), 3.22-3.19 (m, 1H), 3.14-3.10 (m, 2H), 2.70-2.66 (m, 2H), 2.16-2.01 (m, 2H), 1.64-1.61 (m, 1H), 1.44-1.41 (m, 1H), 1.33-1.31 (m, 2H), 1.29-1.27 (m, 1H), MS m/z=589.3 (M+H)+

### Example 189: (S)-3-fluoro-4-(4-((4-(3-hydroxypiperidin-1-yl)-5-(1-(tetrahydro-2H-pyran-4-yl)-1H-pyrazol-4-yl)pyridin-2-yl)amino)pyrimidin-2-yl)-5-methoxybenzaldehyde

The titled compound (95.3 mg) was prepared in accordance with the same procedures as in Example 7, using Intermediates A2-1 and C4-1. (Yield: 30.1%) 1H NMR (CDCl₃, 400 MHz) 9.98 (s, 1H), 8.57 (d, 1H), 8.11 (s, 1H), 7.95 (s, 1H), 7.77 (d, 2H), 7.51 (s, 1H), 7.33-7.30 (m, 2H), 7.27-7.23 (m, 1H), 4.41-4.38 (m, 1H), 4.11-4.10 (m, 2H), 3.89 (s, 3H), 3.82-3.80 (m, 1H), 3.60-3.54 (m, 2H), 3.21-3.19 (m, 1H), 3.01-2.95 (m, 1H), 2.67-2.62 (m, 2H), 2.17-2.11 (m, 4H), 1.90-1.87 (m, 1H), 1.73-1.48 (m, 1H), 1.46-1.24 (m, 2H) δ MS m/z=574.1 (M+H)+

### Example 190: (S)-1-(2-((2-(2-fluoro-6-methoxy-4-((methylamino)methyl)phenyl)pyrimidin-4-yl)amino)-5-(1-(tetrahydro-2H-pyran-4-yl)-1H-pyrazol-4-yl)pyridin-4-yl)piperidin-3-ol

The compound prepared in Example 189 (20.0 mg, 0.035 mmol) was dissolved in methanol (0.5 mL) and then 2.0M methylamine dissolved in tetrahydrofuran (34.8 uL, 0.07 mmol) and sodium acetate (2.8 mg, 0.035 mmol) were added dropwise thereto. The reaction mixture was stirred at room temperature overnight. Then, sodium borohydride (1.3 mg, 0.035 mmol) was added to the reaction mixture, which was then stirred at room temperature for 1 hour. Dichloromethane was added to the reaction mixture and then a saturated sodium hydrogen carbonate solution was added thereto. The organic layer was extracted, dried over anhydrous magnesium sulfate, and then filtered. The filtrate was concentrated and then purified by silica gel column chromatography to prepare the titled compound (10.2 mg). (Yield: 49.7%) 1H NMR (CD₃OD, 400 MHz) δ 8.57 (d, 1H), 8.09 (s, 1H), 7.77 (s, 2H), 7.45-7.44 (m, 1H), 7.31 (s, 1H), 7.30-7.28 (m, 1H), 6.80-6.79 (m, 2H), 4.41-4.40 (m, 1H), 4.39-4.13 (m, 2H), 3.83 (s, 3H), 3.78 (s, 3H), 3.60-3.55 (m, 2H), 3.23-3.21 (m, 1H), 3.02-2.99 (m, 1H), 2.62-2.58 (m, 2H), 2.48-2.38 (m, 2H), 2.17-2.10 (m, 4H), 1.92-1.91 (m, 1H), 1.51-1.48 (m, 1H), 1.45-1.42 (m, 1H) MS m/z=589.3 (M+H)+

### Example 191: 6-((6-(2-fluoro-6-methoxyphenyl)-5-nitropyridin-2-yl)amino)-4-((S)-3-hydroxypiperidin-1-yl)nicotinamide

The titled compound (5.5 mg) was prepared in accordance with the same procedures as in Example 7, using Intermediates A11-2 and C3-1. (Yield: 14.6%) 1H NMR (CD₃OD, 400 MHz) δ 8.43 (s, 1H), 8.41 (d, 1H), 7.92 (s, 1H), 7.52-7.44 (m, 2H), 6.94-6.90 (m, 2H), 3.85-3.81 (m, 1H), 3.76 (s, 3H), 3.28-3.23 (m, 1H), 3.09-3.07 (m, 1H), 2.87-2.79 (m, 1H), 2.77-2.72 (m, 1H), 1.91-1.87 (m, 2H), 1.66-1.63 (m, 1H), 1.53-1.47 (m, 1H) MS m/z=483.2 (M+H)+

### Example 192: 6-((2-(2-fluoro-6-methoxyphenyl)pyrimidin-4-yl)amino)-4-((S)-3-hydr oxypiperidin-1-yl)-N-((R)-1-methylpyrrolidin-3-yl)nicotinamide

### Step 1: methyl (S)-6-((2-(2-fluoro-6-methoxyphenyl)pyrimidin-4-yl)amino)-4-(3-hydroxypiperidin-1-yl)nicotinate

The titled compound (99.8 mg) was prepared in accordance with the same procedures as in Example 7, using Intermediates A11-1 and C1-1. (Yield: 19.3%) 1H NMR (CD₃OD, 400 MHz) δ 8.50-8.45 (m, 2H), 7.69 (s, 1H), 7.49-7.41 (m, 2H), 6.95 (d, 1H), 6.84 (t, 1H), 3.88 (s, 3H), 3.81 (s, 3H), 3.75-3.71 (m, 1H), 3.39-3.36 (m, 1H), 2.86-2.80 (m, 1H), 2.72-2.67 (m, 1H), 2.01-1.96 (m, 1H), 1.77-1.73 (m, 1H), 1.63-1.60 (m, 1H), 1.42-1.39 (m, 1H)

### Step 2: (S)-6-((2-(2-fluoro-6-methoxyphenyl)pyrimidin-4-yl)amino)-4-(3-hydroxypiperidin-1-yl)nicotinic acid

Methyl (S)-6-((2-(2-fluoro-6-methoxyphenyl)pyrimidin-4-yl)amino)-4-(3-hydroxypiperidin-1-yl)nicotinate (50.0 mg, 0.11 mmol) was dissolved in methanol (2.0 ml) and then 1.0 N sodium hydroxide solution (0.55 mL) was added dropwise thereto. Then, the mixed solution was stirred at 60 °C overnight. After completion of the reaction, the reaction mixture was cooled to room temperature and then neutralized with 1.0 N hydrochloric acid solution. The aqueous layer was extracted with dichloromethane. The extracted organic layer was dried over anhydrous magnesium sulfate and then filtered. The filtrate was concentrated and then dried to prepare the titled compound (54.1 mg), without further purification. (Yield: 98.3%) 1H NMR (CD₃OD, 400 MHz) δ 8.78 (d, 1H), 8.53 (s, 1H), 7.70-7.64 (m, 1H), 7.40 (d, 1H), 7.17-7.13 (m, 2H), 7.02 (t, 1H), 3.95-3.92 (m, 4H), 3.73-3.69 (m, 1H), 3.62-3.58 (m, 1H), 3.50-3.45 (m, 1H), 2.09-1.95 (m, 2H), 1.70-1.67 (m, 2H)

### Step 3: 6-((2-(2-fluoro-6-methoxyphenyl)pyrimidin-4-yl)amino)-4-((S)-3-hydroxypiperidin-1-yl)-N-((R)-1-methylpyrrolidin-3-yl)nicotinamide

(S)-6-((2-(2-Fluoro-6-methoxyphenyl)pyrimidin-4-yl)amino)-4-(3-hydroxypiperidin-1-yl)nicotinic acid (30.0 mg, 0.06 mmol) was dissolved in anhydrous dimethylformamide (1.0 mL) and then 1-[bis(dimethylamino)methylene]-1H-1,2,3-triazolo[4,5-b]pyridinium-3-oxide hexafluorophosphate (HATU, 45.7 mg, 0.12 mmol) and diisopropylethylamine (31 uL, 0.18 mmol) were added dropwise thereto. The reaction mixture was stirred at room temperature for 15 minutes and then (3R)-1-methylpyrrolidin-3-amine (9.03 mg, 0.09 mmol) was added dropwise thereto. The reaction mixture was stirred at 60 °C overnight. Water was added to the reaction mixture and then the aqueous layer was extracted with ethyl acetate. The extracted organic layer was dried over anhydrous magnesium sulfate and then filtered. The filtrate was concentrated and then purified by silica gel column chromatography to prepare the titled compound (2.8 mg). (Yield: 8.94%) 1H NMR (CD₃OD, 400 MHz) δ 8.49-8.42 (m, 3H), 7.79 (s, 1H), 7.52-7.46 (m, 2H), 6.98 (d, 1H), 6.86 (t, 1H), 4.59-4.51 (m, 1H), 3.83 (s, 3H), 3.52-3.42 (m, 2H), 3.24-3.21 (m, 2H), 3.15-3.09 (m, 1H), 2.95-2.82 (m, 4H), 2.60-2.51 (m, 1H), 2.28-2.13 (m, 1H), 1.90-1.88 (m, 2H), 1.65-1.60 (m, 2H) MS m/z=522.2 (M+H)+

### Example 193: 6-((5-amino-6-(2-fluoro-6-methoxyphenyl)pyridin-2-yl)amino)-4-((S)-3-hydroxypiperidin-1-yl)nicotinamide

The titled compound (5.5 mg) was prepared in accordance with the same procedures as in Example 150, using Intermediates A11-2 and C3-1. (Yield: 48.9%) 1H NMR (CD₃OD, 400 MHz) δ 8.38 (s, 1H), 7.49-7.43 (m, 2H), 7.30-7.21 (m, 2H), 6.99 (d, 1H), 6.89 (t, 1H), 3.88-3.81 (m, 4H), 3.25-3.21 (m, 1H), 3.10-3.02 (m, 1H), 2.87-2.79 (m, 1H), 2.75-2.70 (m, 1H), 1.96-1.85 (m, 2H), 1.72-1.62 (m, 1H), 1.54-1.46 (m, 1H) MS m/z=453.2 (M+H)+

### Example 194: 6-((6-(2-fluoro-6-methoxyphenyl)-5-nitropyridin-2-yl)amino)-N-((1R,3R)-3-hydroxycyclopentyl)-4-((S)-3-hydroxypiperidin-1-yl)nicotinamide

The titled compound (3.3 mg) was prepared in accordance with the same procedures as in Example 192, using Intermediates A11-1 and C3-1 and changing the amine reagent to (1R,3R)-3-aminocyclopentan-1-ol hydrochloride. (Yield: 14.1%) 1H NMR (CD₃OD, 400 MHz) δ 8.40 (d, 1H), 8.31 (s, 1H), 7.91 (d, 1H), 7.49-7.42 (m, 2H), 6.94-6.89 (m, 2H), 4.55-4.52 (m, 1H), 4.39-4.37 (m, 1H), 3.83-3.81 (m, 1H), 3.76 (s, 3H), 3.27-3.18 (m, 1H), 3.08-2.96 (m, 1H), 2.90-2.82 (m, 1H), 2.79-2.72 (m, 1H), 2.30-2.22 (m, 1H), 2.11-2.06 (m, 2H), 1.89-1.84 (m, 3H), 1.64-1.53 (m, 4H) MS m/z=568.2 (M+H)+

### Example 195: 6-((6-(2-fluoro-6-methoxyphenyl)-5-nitropyridin-2-yl)amino)-N-((1R,3S)-3-hydroxycyclopentyl)-4-((S)-3-hydroxypiperidin-1-yl)nicotinamide

The titled compound (4.5 mg) was prepared in accordance with the same procedures as in Example 192, using Intermediates A11-1 and C3-1 and changing the amine reagent to (1S,3R)-3-aminocyclopentan-1-ol hydrochloride. (Yield: 19.2%) 1H NMR (CD₃OD, 400 MHz) δ 8.40 (d, 1H), 8.40 (d, 1H), 8.37 (s, 1H), 7.92 (s, 1H), 7.51-7.42 (m, 2H), 6.94-6.89 (m, 2H), 4.37-4.31 (m, 2H), 3.88-3.81 (m, 1H), 3.76 (s, 3H), 3.76 (s, 1H), 3.13-3.04 (m, 1H), 2.86-2.77 (m, 1H), 2.75-2.68 (m, 1H), 2.30-2.22 (m, 1H), 2.14-2.07 (m, 1H), 1.95-1.77 (m, 5H), 1.69-1.64 (m, 2H), 1.49-1.42 (m, 1H) MS m/z=568.2 (M+H)+

### Example 196: 6-((6-(2-fluoro-6-methoxyphenyl)-5-nitropyridin-2-yl)amino)-N-(3-hydroxycyclobutyl)-4-((S)-3-hydroxypiperidin-1-yl)nicotinamide

The titled compound (3.6 mg) was prepared in accordance with the same procedures as in Example 192, using Intermediates A11-1 and C3-1 and changing the amine reagent to 3-aminocyclobutanol. (Yield: 15.8%) 1H NMR (CD₃OD, 400 MHz) δ 8.42-8.33 (m, 2H), 7.93-7.90 (m, 1H), 7.50-7.43 (m, 2H), 6.94-6.88 (m, 2H), 4.29-4.25 (m, 1H), 4.15-4.09 (m, 1H), 3.87-3.82 (m, 1H), 3.76 (s, 3H), 3.29-3.20 (m, 1H), 3.10-3.02 (m, 1H), 2.96-2.82 (m, 1H), 2.78-2.72 (m, 1H), 2.15-2.10 (m, 2H), 1.97-1.83 (m, 2H), 1.64-1.47 (m, 4H) MS m/z=552.9 (M+H)+

### Example 197: 6-((6-(2-fluoro-6-methoxyphenyl)-5-nitropyridin-2-yl)amino)-N-((1R,2R)-2-hydroxycyclobutyl)-4-((S)-3-hydroxypiperidin-1-yl)nicotinamide

The titled compound (2.1 mg) was prepared in accordance with the same procedures as in Example 192, using Intermediates A11-1 and C3-1 and changing the amine reagent to (1R,2R)-2-aminocyclobutanol hydrochloride. (Yield: 9.19%) 1H NMR (CD₃OD, 400 MHz) δ 8.40 (d, 1H), 8.34-8.32 (m, 1H), 7.92 (d, 1H), 7.50-7.43 (m, 2H), 6.94-6.90 (m, 2H), 4.57-4.43 (m, 1H), 4.06-4.00 (m, 1H), 3.88-3.80 (m, 1H), 3.76 (s, 3H), 3.26-3.18 (m, 1H), 3.07-3.00 (m, 1H), 2.93-2.85 (m, 1H), 2.81-2.73 (m, 2H), 2.46-2.37 (m, 1H), 2.01-1.89 (m, 4H), 1.67-1.49 (m, 2H) MS m/z=552.9 (M+H)+

### Example 198: N-(3,3-difluorocyclobutyl)-6-((6-(2-fluoro-6-methoxyphenyl)-5-nitropyridin-2-yl)amino)-4-((S)-3-hydroxypiperidin-1-yl)nicotinamide

The titled compound (3.5 mg) was prepared in accordance with the same procedures as in Example 192, using Intermediates A11-1 and C3-1 and changing the amine reagent to 3,3-difluorocyclobutanamine hydrochloride. (Yield: 14.8%) 1H NMR (CD₃OD, 400 MHz) δ 8.42-8.37 (m, 2H), 7.95 (d, 1H), 7.50-7.43 (m, 2H), 6.94-6.88 (m, 2H), 4.33-4.31 (m, 1H), 3.85-3.82 (m, 1H), 3.87-3.82 (m, 1H), 3.76 (s, 3H), 3.20-3.12 (m, 1H), 3.06-2.98 (m, 3H), 2.96-2.84 (m, 1H), 2.82-2.71 (m, 3H), 2.91-2.82 (m, 2H), 1.64-1.53 (m, 2H) MS m/z=573.9 (M+H)+

### Example 199: N-((1R,3R)-3-aminocyclopentyl)-6-((6-(2-fluoro-6-methoxyphenyl)-5-nitropyridin-2-yl)amino)-4-((S)-3-hydroxypiperidin-1-yl)nicotinamide hydrochloride

### Step 1: tert-butyl ((1R,3R)-3-(6-((6-(2-fluoro-6-methoxyphenyl)-5-nitropyridin-2-yl)amino)-4-((S)-3-hydroxypiperidin-1-yl)nicotinamido)cyclopentyl)carbamate

The titled compound (11.3 mg) was prepared in accordance with the same procedures as in Example 192, using Intermediates A11-1 and C3-1 and changing the amine reagent to tert-butyl N-[(1R,3R)-3-aminocyclopentyl]carbamate. (Yield: 41.0%) 1H NMR (CD₃OD, 400 MHz) δ 8.40 (d, 1H), 8.31 (s, 1H), 7.91 (s, 1H), 7.50-7.44 (m, 2H), 6.94-6.89 (m, 2H), 4.43-4.39 (m, 1H), 4.08-4.03 (m, 1H), 3.84-3.80 (m, 1H), 3.76 (s, 3H), 3.26-3.18 (m, 1H), 3.09-3.00 (m, 1H), 2.91-2.82 (m, 1H), 2.78-2.69 (m, 1H), 2.25-2.18 (m, 1H), 2.16-2.05 (m, 1H), 1.98-1.88 (m, 4H), 1.62-1.51 (m, 4H), 1.46 (s, 9H)

### Step 2: N-((1R,3R)-3-aminocyclopentyl)-6-((6-(2-fluoro-6-methoxyphenyl)-5-nitropyridin-2-yl)amino)-4-((S)-3-hydroxypiperidin-1-yl)nicotinamide hydrochloride

tert-Butyl ((1R,3R)-3-(6-((6-(2-fluoro-6-methoxyphenyl)-5-nitropyridin-2-yl)amino)-4-((S)-3-hydroxypiperidin-1-yl)nicotinamido)cyclopentyl)carbamate (11.3 mg, 0.017 mmol) was dissolved in dichloromethane (1 ml) and then 4.0 M hydrogen chloride solution dissolved in 1,4-dioxane (20 μl, 0.085 mmol) was added dropwise thereto. Then, the mixed solution was stirred at room temperature for 4 hours. After completion of the reaction, the reaction mixture was concentrated and the resulting solid was dried to prepare the titled compound (5.5 mg). (Yield: 50.8%) 1H NMR (CD₃OD, 400 MHz) δ 8.57 (d, 1H), 8.07 (s, 1H), 7.54 (q, 1H), 7.25 (d, 1H), 7.01-6.96 (m, 2H), 6.84 (s, 1H), 4.48-4.46 (m, 1H), 3.86-3.81 (m, 2H), 3.79 (s, 3H), 3.67-3.64 (m, 1H), 3.57-3.52 (m, 1H), 3.39-3.37 (m, 1H), 3.26-3.21 (m, 1H), 2.34-2.28 (m, 2H), 2.15 (t, 2H), 2.04-1.96 (m, 2H), 1.78-1.64 (m, 4H) MS m/z=565.9 (M+H)+

### Example 200: N-((1R,3S)-3-aminocyclopentyl)-6-((6-(2-fluoro-6-methoxyphenyl)-5-nitropyridin-2-yl)amino)-4-((S)-3-hydroxypiperidin-1-yl)nicotinamide

The titled compound (4.4 mg) was prepared in accordance with the same procedures as in Example 199, changing the amine reagent to tert-butyl N-[(1R,3S)-3-aminocyclopentyl]carbamate. (Yield: 40.6%) 1H NMR (CD₃OD, 400 MHz) δ 8.57 (d, 1H), 8.08 (s, 1H), 7.54 (q, 1H), 7.25 (d, 1H), 7.01-6.94 (m, 2H), 6.86 (s, 1H), 4.29-4.27 (m, 1H), 3.88-3.84 (m, 1H), 3.79 (s, 3H), 3.69-3.64 (m, 2H), 3.56-3.50 (m, 1H), 3.39-3.37 (m, 1H), 3.26-3.21 (m, 1H), 2.65-2.62 (m, 1H), 2.19-2.15 (m, 2H), 2.04-1.90 (m, 4H), 1.76-1.67 (m, 3H) MS m/z=566.0 (M+H)+

### Example 201: 6-((5-amino-6-(2-fluoro-6-methoxyphenyl)pyridin-2-yl)amino)-N-((1R,3R)-3-hydroxycyclopentyl)-4-((S)-3-hydroxypiperidin-1-yl)nicotinamide

6-((6-(2-Fluoro-6-methoxyphenyl)-5-nitropyridin-2-yl)amino)-N-((1R,3R)-3-hydroxycyclopentyl)-4-((S)-3-hydroxypiperidin-1-yl)nicotinamide (Example 194, 7.0 mg, 0.012 mmol) was dissolved in methanol (1.0 mL) and then Pd/C (5.26 mg, 0.05 mmol) was added dropwise thereto. The reaction mixture was stirred at room temperature for 3 hours under a pressure of a balloon filled with hydrogen gas. After completion of the reaction, the reaction mixture was filtered through a Celite filter. The filtrate was concentrated and then purified by silica gel column chromatography to prepare the titled compound (2.1 mg). (Yield: 31.7%) 1H NMR (CD₃OD, 400 MHz) δ 8.28 (s, 1H), 7.49-7.45 (m, 2H), 7.29 (d, 1H), 7.23-7.20 (m, 1H), 6.99 (d, 1H), 6.89 (t, 1H), 4.54-4.50 (m, 1H), 4.42-4.38 (m, 1H), 3.85-3.81 (m, 4H), 3.25-3.19 (m, 1H), 3.03-2.96 (m, 1H), 2.87-2.81 (m, 1H), 2.77-2.69 (m, 1H), 2.30-2.22 (m, 1H), 2.11-2.06 (m, 2H), 1.90-1.85 (m, 3H), 1.64-1.56 (m, 4H) MS m/z=538.3 (M+H)+

### Example 202: 6-((5-amino-6-(2-fluoro-6-methoxyphenyl)pyridin-2-yl)amino)-N-((1R,3S)-3-hydroxycyclopentyl)-4-((S)-3-hydroxypiperidin-1-yl)nicotinamide

The titled compound (2.2 mg) was prepared in accordance with the same procedures as in Example 201, using the compound of Example 195. (Yield: 33.2%) 1H NMR (CD₃OD, 400 MHz) δ 8.33 (s, 1H), 7.50-7.42 (m, 2H), 7.29 (d, 1H), 7.24-7.21 (m, 1H), 6.98 (d, 1H), 6.89 (t, 1H), 4.36-4.30 (m, 2H), 4.42-4.38 (m, 1H), 3.85-3.82 (m, 4H), 3.25-3.23 (m, 1H), 3.08-3.02 (m, 1H), 2.83-2.75 (m, 1H), 2.70-2.63 (m, 1H), 2.34-2.28 (m, 1H), 2.11-2.07 (m, 1H), 1.91-1.76 (m, 5H), 1.69-1.64 (m, 2H), 1.52-1.43 (m, 1H) MS m/z=538.3 (M+H)+

### Example 203: 6-((6-(2-fluoro-6-methoxyphenyl)-5-nitropyridin-2-yl)amino)-4-((S)-3-hydroxypiperidin-1-yl)-N-((R)-1-methylpyrrolidin-3-yl)nicotinamide

The titled compound (2.6 mg) was prepared in accordance with the same procedures as in Example 192, using Intermediates A11-1 and C3-1 and changing the amine reagent to (3R)-1-methylpyrrolidin-3-amine. (Yield: 22.2%) 1H NMR (CD₃OD, 400 MHz) δ 8.44-8.38 (m, 2H), 7.95 (s, 1H), 7.52-7.45 (m, 2H), 6.96-6.92 (m, 2H), 4.57-4.51 (m, 1H), 3.85-3.82 (m, 1H), 3.78 (s, 3H), 3.27-3.20 (m, 1H), 3.09-3.02 (m, 1H), 2.98-2.93 (m, 1H), 2.89-2.82 (m, 1H), 2.80-2.74 (m, 2H), 2.67-2.59 (m, 2H), 2.43-2.38 (m, 4H), 1.92-1.86 (m, 3H), 1.68-1.59 (m, 1H), 1.58-1.49 (m, 1H) MS m/z=566.2 (M+H)+

### Example 204: N-(4-((3S,4R)-3-amino-4-methylpiperidin-1-yl)-5-(1-(difluoromethyl)-1H-pyrazol-4-yl)pyridin-2-yl)-2-(2-fluoro-6-methoxyphenyl)pyrimidin-4-amine hydrochloride

The titled compound (7.4 mg) was prepared in accordance with the same procedures as in Example 52, using Intermediates B1-3 and C1-1. (Yield: 62.7%) 1H NMR (CD₃OD, 400 MHz) δ 8.75-8.72 (m, 1H), 8.52 (s, 1H), 8.26 (s, 1H), 8.11 (s, 1H), 7.68-7.65 (m, 2H), 7.61 (t, 1H), 7.28-7.22 (m, 1H), 7.15-7.13 (m, 1H), 7.03-6.99 (m, 1H), 3.96 (s, 3H), 3.91-3.82 (m, 1H), 3.62-3.56 (m, 1H), 3.08-2.74 (m, 3H), 1.87-1.74 (m, 2H), 1.56-1.50 (m, 1H), 1.16 (d, 3H) MS m/z=525.2 (M+H)+

### Example 205: N-(4-((3S,5S)-3-amino-5-methylpiperidin-1-yl)-5-(1-(difluoromethyl)-1H-pyrazol-4-yl)pyridin-2-yl)-2-(2-fluoro-6-methoxyphenyl)pyrimidin-4-amine hydrochloride

The titled compound (2.9 mg) was prepared in accordance with the same procedures as in Example 52, using Intermediates B1-4 and C1-1. (Yield: 24.6%) 1H NMR (CD₃OD, 400 MHz) δ 8.74-8.68 (m, 1H), 8.52 (s, 1H), 8.25-8.23 (m, 1H), 8.11 (s, 1H), 7.68-7.66 (m, 2H), 7.61 (t, 1H), 7.30-7.23 (m, 1H), 7.14-7.12 (m, 1H), 7.02-6.98 (m, 1H), 3.94 (s, 3H), 3.70-3.64 (m, 1H), 3.50-3.36 (m, 3H), 2.63-2.57 (m, 1H), 2.05-2.01 (m, 1H), 1.97-1.94 (m, 1H), 1.16 (d, 1H), 0.93 (d, 3H) MS m/z=525.2 (M+H)+

### Example 206: (S)-N-(4-(3-amino-3-methylpiperidin-1-yl)-5-(1-(difluoromethyl)-1H-pyrazol-4-yl)pyridin-2-yl)-2-(2-fluoro-6-methoxyphenyl)pyrimidin-4-amine hydrochloride

The titled compound (2.1 mg) was prepared in accordance with the same procedures as in Example 52, using Intermediates B1-5 and C1-1. (Yield: 17.8%) 1H NMR (CD₃OD, 400 MHz) δ 8.74-8.69 (m, 1H), 8.50 (s, 1H), 8.23 (s, 1H), 8.07 (s, 1H), 7.67-7.64 (m, 2H), 7.62 (t, 1H), 7.38-7.30 (m, 1H), 7.15-7.12 (m, 1H), 7.01-6.98 (m, 1H), 3.95 (s, 3H), 3.49-3.36 (m, 1H), 3.22-3.03 (m, 3H), 1.98-1.72 (m, 4H), 1.39 (s, 3H) MS m/z=525.2 (M+H)+

### Example 207: N-(4-((3R,4S)-3-amino-4-fluoropiperidin-1-yl)-5-(1-(difluoromethyl)-1H-pyrazol-4-yl)pyridin-2-yl)-2-(2-fluoro-6-methoxyphenyl)pyrimidin-4-amine hydrochloride

The titled compound (7.6 mg) was prepared in accordance with the same procedures as in Example 52, using Intermediates B1-6 and C1-1. (Yield: 58.8%) 1H NMR (CD₃OD, 400 MHz) δ 8.67 (d, 1H), 8.54 (s, 1H), 8.33 (s, 1H), 8.16 (s, 1H), 7.68-7.62 (m, 2H), 7.60 (t, 1H), 7.35-7.26 (m, 1H), 7.13 (d, 1H), 7.00 (t, 1H), 5.22-5.18 (m, 1H), 5.07-5.04 (m, 1H), 3.94 (s, 3H), 3.76-3.60 (m, 2H), 3.54-3.50 (m, 1H), 3.15-3.09 (m, 2H), 2.21-2.17 (m, 1H) MS m/z=529.2 (M+H)+

### Example 208: N-(4-((3R,4R)-3-amino-4-fluoropiperidin-1-yl)-5-(1-(difluoromethyl)-1H-pyrazol-4-yl)pyridin-2-yl)-2-(2-fluoro-6-methoxyphenyl)pyrimidin-4-amine hydrochloride

The titled compound (1.6 mg) was prepared in accordance with the same procedures as in Example 52, using Intermediates B1-7 and C1-1. (Yield: 12.4%) 1H NMR (CD₃OD, 400 MHz) δ 8.69 (d, 1H), 8.55 (s, 1H), 8.33 (s, 1H), 8.16 (s, 1H), 7.69-7.61 (m, 3H), 7.28-7.23 (m, 1H), 7.13 (d, 1H), 7.01 (t, 1H), 4.68-4.65 (m, 1H), 3.94 (s, 3H), 3.75-3.69 (m, 1H), 3.58-3.49 (m, 2H), 3.06-3.00 (m, 1H), 2.96-2.89 (m, 1H), 2.30-2.27 (m, 1H), 2.05-1.94 (m, 1H) MS m/z=529.2 (M+H)+

### Example 209: (3R,4R)-3-amino-1-(5-(1-(difluoromethyl)-1H-pyrazol-4-yl)-2-((2-(2-fluoro-6-methoxyphenyl)pyrimidin-4-yl)amino)pyridin-4-yl)piperidin-4-ol hydrochloride

The titled compound (4.4 mg) was prepared in accordance with the same procedures as in Example 52, using Intermediates B1-8 and C1-1. (Yield: 34.2%) 1H NMR (CD₃OD, 400 MHz) δ 8.72-8.68 (m, 1H), 8.53 (s, 1H), 8.28 (s, 1H), 8.12 (s, 1H), 7.67-7.61 (m, 3H), 7.28-7.20 (m, 1H), 7.13 (d, 1H), 7.00 (t, 1H), 3.94 (s, 3H), 3.80-3.74 (m, 1H), 3.74-3.61 (m, 1H), 3.60-3.51 (m, 1H), 3.18-3.11 (m, 1H), 2.98-2.85 (m, 2H), 2.11-2.03 (m, 1H), 1.75-1.69 (m, 1H) MS m/z=527.2 (M+H)+

### Example 210: N-(4-((3S,5S)-3-amino-5-methylpiperidin-1-yl)-5-(1-(difluoromethyl)-1H-pyrazol-4-yl)pyridin-2-yl)-2-(2,4-difluoro-6-methoxyphenyl)pyrimidin-4-amine hydrochloride

The titled compound (4.4 mg) was prepared in accordance with the same procedures as in Example 52, using Intermediates B1-4 and C1-7. (Yield: 45.5%) 1H NMR (CD₃OD, 400 MHz) δ 8.75 (d, 1H), 8.54 (s, 1H), 8.21 (s, 1H), 8.10 (s, 1H), 7.79-7.49 (m, 2H), 7.23 (s, 1H), 7.01 (d, 1H), 6.90-6.85 (m, 1H), 3.95 (s, 3H), 3.75-3.69 (m, 1H), 3.48-3.44 (m, 3H), 2.72-2.67 (m, 1H), 2.06-1.95 (m, 2H), 1.76-1.70 (m, 1H), 0.90 (d, 3H) MS m/z=543.2 (M+H)+

### Example 211: N-(4-((3S,4R)-3-amino-4-methylpiperidin-1-yl)-5-(1-(difluoromethyl)-1H-pyrazol-4-yl)pyridin-2-yl)-2-(2,4-difluoro-6-methoxyphenyl)pyrimidin-4-amine hydrochloride

The titled compound (5.7 mg) was prepared in accordance with the same procedures as in Example 52, using Intermediates B1-3 and C1-7. (Yield: 58.9%) 1H NMR (CD₃OD, 400 MHz) δ 8.75 (d, 1H), 8.53 (s, 1H), 8.24 (s, 1H), 8.10 (s, 1H), 7.77-7.47 (m, 2H), 7.22 (s, 1H), 7.00 (d, 1H), 6.90-6.85 (m, 1H), 3.95 (s, 3H), 3.85 (d, 1H), 3.59 (d, 1H), 3.08-3.04 (m, 1H), 3.00-2.88 (m, 2H), 1.87-1.78 (m, 2H), 1.55-1.47 (m, 1H), 1.17 (d, 3H) MS m/z=543.2 (M+H)+

### Example 212: (S)-N-(4-(3-amino-3-methylpiperidin-1-yl)-5-(1-(difluoromethyl)-1H-pyrazol-4-yl)pyridin-2-yl)-2-(2,4-difluoro-6-methoxyphenyl)pyrimidin-4-amine hydrochloride

The titled compound (3.6 mg) was prepared in accordance with the same procedures as in Example 52, using Intermediates B1-5 and C1-7. (Yield: 37.2%) 1H NMR (CD₃OD, 400 MHz) δ 8.73 (d, 1H), 8.49 (s, 1H), 8.22 (s, 1H), 8.06 (s, 1H), 7.77-7.47 (m, 2H), 7.26 (s, 1H), 7.00 (d, 1H), 6.90-6.85 (m, 1H), 3.94 (s, 3H), 3.45-3.39 (m, 1H), 3.25-3.22 (m, 2H), 3.20-3.12 (m, 1H), 1.93-1.85 (m, 2H), 1.83-1.76 (m, 2H), 1.40 (s, 3H) MS m/z=543.2 (M+H)+

### Example 213: (3R,4R)-3-amino-1-(2-((2-(2,4-difluoro-6-methoxyphenyl)pyrimidin-4-yl)amino)-5-(1-(difluoromethyl)-1H-pyrazol-4-yl)pyridin-4-yl)piperidin-4-ol hydrochloride

The titled compound (2.1 mg) was prepared in accordance with the same procedures as in Example 52, using Intermediates B1-8 and C1-7. (Yield: 21.6%) 1H NMR (CD₃OD, 400 MHz) δ 8.73 (d, 1H), 8.53 (s, 1H), 8.26 (s, 1H), 8.11 (s, 1H), 7.76-7.47 (m, 2H), 7.21 (s, 1H), 6.99 (d, 1H), 6.89-6.84 (m, 1H), 3.94 (s, 3H), 3.80 (d, 1H), 3.69-3.58 (m, 2H), 3.18-3.12 (m, 1H), 3.02-2.91 (m, 2H), 2.13-2.05 (m, 1H), 1.77-1.67 (m, 1H) MS m/z=545.2 (M+H)+

### Example 214: (1S,4S)-4-((5-(1-(difluoromethyl)-1H-pyrazol-4-yl)-2-((2-(2-fluoro-6-methoxyphenyl)pyrimidin-4-yl)amino)pyridin-4-yl)(methyl)amino)cyclohexan-1-ol

The titled compound (5.7 mg) was prepared in accordance with the same procedures as in Example 7, using Intermediates B3-35 and C1-1. (Yield: 15.4%) 1H NMR (CD₃OD, 400 MHz) δ 8.45 (d, 1H), 8.28 (s, 1H), 8.02 (s, 1H), 7.95 (s, 1H), 7.69-7.39 (m, 4H), 6.98 (d, 1H), 6.86 (t, 1H), 3.83 (s, 3H), 3.79-3.74 (m, 1H), 3.28-3.22 (m, 1H), 2.65 (s, 3H), 1.93-1.85 (m, 2H), 1.70 (d, 2H), 1.35-1.32 (m, 3H) MS m/z=540.3 (M+H)+

### Example 215: N²-(4-((3S,4R)-3-amino-4-methylpiperidin-1-yl)-5-(1-(difluoromethyl)-1H-pyrazol-4-yl)pyridin-2-yl)-6-(2,4-difluoro-6-methoxyphenyl)pyridin-2,5-diamine hydrochloride

The titled compound (1.9 mg) was prepared in accordance with the same procedures as in Example 151, using Intermediates B1-3 and C3-2. (Yield: 10.3%) 1H NMR (CD₃OD, 400 MHz) δ 8.46 (s, 1H), 8.05 (s, 2H), 7.82 (d, 1H), 7.60 (t, 1H), 7.31 (d, 1H), 6.97-6.92 (m, 2H), 6.85 (t, 1H), 3.91 (s, 3H), 3.82 (d, 3H), 3.80 (d, 1H), 3.54 (d, 1H), 3.10-3.02 (m, 1H), 2.90 (t, 2H), 1.84-1.76 (m, 2H), 1.50-1.47 (m, 1H), 1.17 (d, 3H) MS m/z=557.2 (M+H)+

### Example 216: N²-(4-((3R,4R)-3-amino-4-fluoropiperidin-1-yl)-5-(1-(difluoromethyl)-1H-pyrazol-4-yl)pyridin-2-yl)-6-(2,4-difluoro-6-methoxyphenyl)pyridin-2,5-diamine hydrochloride

The titled compound (4.7 mg) was prepared in accordance with the same procedures as in Example 151, using Intermediates B1-7 and C3-2. (Yield: 25.4%) 1H NMR (CD₃OD, 400 MHz) δ 8.50 (s, 1H), 8.07 (s, 2H), 7.80 (d, 1H), 7.60 (t, 1H), 7.30 (d, 1H), 6.97-6.94 (m, 2H), 6.84 (t, 1H), 4.89-4.64 (m, 1H), 3.90-3.84 (m, 4H), 3.68-3.50 (m, 2H), 3.07-2.97 (m, 2H), 2.31-2.17 (m, 1H), 1.96-1.82 (m, 1H), 1.84-1.76 (m, 2H), 1.50-1.47 (m, 1H), 1.17 (d, 3H) MS m/z=561.2 (M+H)+

### Example 217: (S)-1-(2-((2-(2,4-difluoro-6-methoxyphenyl)pyrimidin-4-yl)amino)-5-((1-(difluoromethyl)-1H-pyrazol-4-yl)ethynyl)pyridin-4-yl)piperidin-3-ol

The titled compound (14.8 mg) was prepared in accordance with the same procedures as in Example 7, using Intermediates A6-3 and C1-7. (Yield: 31.7%) 1H NMR (CDCl₃, 400 MHz) δ 8.64 (d, 1H), 8.32 (s, 1H), 8.12 (s, 1H), 7.88 (s, 1H), 7.78 (s, 1H), 7.41-7.32 (m, 2H), 7.30-7.28 (m, 0.2H), 7.23-7.18 (m, 0.6H), 7.07-7.04 (m, 0.2H), 6.63-6.51 (m, 2H), 4.05-3.95 (m, 1H), 3.86 (s, 3H), 3.53-3.44 (m, 2H), 3.37-3.19 (m, 2H), 2.35-2.19 (m, 1H), 2.04-1.90 (m, 1H), 1.90-1.82 (m, 1H), 1.79-1.67 (m, 2H) MS m/z=553.9 (M+H)+

### Example 218: (S)-1-(2-((2-(2,4-difluoro-6-methoxyphenyl)pyrimidin-4-yl)amino)-5-((1-(trifluoromethyl)-1H-pyrazol-4-yl)ethynyl)pyridin-4-yl)piperidin-3-ol

The titled compound (3.1 mg) was prepared in accordance with the same procedures as in Example 7, using Intermediates A6-1 and C1-7. (Yield: 6.4%) 1H NMR (CDCl₃, 400 MHz) δ 8.65 (d, 1H), 8.30 (s, 1H), 8.12 (s, 1H), 7.96 (s, 1H), 7.88 (s, 1H), 7.40-7.32 (m, 2H), 6.60-6.51 (m, 2H), 4.03-3.94 (m, 1H), 3.85 (s, 3H), 3.54-3.44 (m, 2H), 3.34-3.22 (m, 2H), 2.02 (s, 1H), 1.92-1.82 (m, 1H), 1.78-1.64 (m, 2H) MS m/z=571.9 (M+H)+

### Example 219: (S)-1-(2-((2-(2,4-difluoro-6-methoxyphenyl)pyrimidin-4-yl)amino)-5-((1-(2,2-difluoroethyl)-1H-pyrazol-4-yl)ethynyl)pyridin-4-yl)piperidin-3-ol

The titled compound (15.3 mg) was prepared in accordance with the same procedures as in Example 7, using Intermediates A6-4 and C1-7. (Yield: 31.9%) 1H NMR (CDCl₃, 400 MHz) δ 8.61 (d, 1H), 8.31 (s, 1H), 8.12 (s, 1H), 7.79 (s, 2H), 7.39 (s, 2H), 6.62-6.48 (m, 2H), 6.29-5.96 (m, 1H), 4.56-4.42 (m, 2H), 4.05-3.94 (m, 1H), 3.88 (s, 3H), 3.52-3.43 (m, 2H), 3.37-3.17 (m, 2H), 2.52-2.28 (m, 1H), 2.03-1.90 (m, 1H), 1.90-1.80 (m, 1H), 1.80-1.63 (m, 2H) MS m/z=567.9 (M+H)+

### Example 220: (S)-1-(2-((2-(2,4-difluoro-6-methoxyphenyl)pyrimidin-4-yl)amino)-5-((1-(2,2,2-trifluoroethyl)-1H-pyrazol-4-yl)ethynyl)pyridin-4-yl)piperidin-3-ol

The titled compound (9.6 mg) was prepared in accordance with the same procedures as in Example 7, using Intermediates A6-5 and C1-7. (Yield: 19.4%) 1H NMR (CDCl₃, 400 MHz) δ 8.61 (d, 1H), 8.32 (s, 1H), 8.17 (s, 1H), 7.83 (d, 2H), 7.39-7.30 (m, 2H), 6.59-6.50 (m, 2H), 4.80 (q, 2H), 4.05-3.93 (m, 1H), 3.84 (s, 3H), 3.51-3.41 (m, 2H), 3.36-3.19 (m, 2H), 2.02-1.92 (m, 1H), 1.91-1.80 (m, 1H), 1.75-1.65 (m, 2H) MS m/z=585.9 (M+H)+

### Example 221: (3S)-1-(5-((1-(2,2-difluoroethyl)-1H-pyrazol-4-yl)ethynyl)-2-((6-(2-fluoro-6-methoxyphenyl)-5-nitropyridin-2-yl)amino)pyridin-4-yl)piperidin-3-ol

The titled compound (57.1 mg) was prepared in accordance with the same procedures as in Example 7, using Intermediates A6-4 and C3-1. (Yield: 63.3%) 1H NMR (CDCl₃, 400 MHz) δ 8.43 (d, 1H), 8.28 (s, 1H), 8.05 (s, 1H), 7.79 (s, 2H), 7.56-7.49 (m, 1H), 7.46-7.34 (m, 2H), 6.92 (t, 1H), 6.82 (d, 1H), 6.30-5.94 (m, 1H), 4.56-4.42 (m, 2H), 4.04-3.91 (m, 1H), 3.80 (s, 3H), 3.52-3.34 (m, 2H), 3.33-3.14 (m, 2H), 2.34-2.15 (m, 1H), 2.03-1.89 (m, 1H), 1.89-1.77 (m, 1H), 1.77-1.66 (m, 2H) MS m/z=593.9 (M+H)+

### Example 222: (3S)-1-(5-((1-(difluoromethyl)-1H-pyrazol-4-yl)ethynyl)-2-((6-(2-fluoro-6-methoxyphenyl)-5-nitropyridin-2-yl)amino)pyridin-4-yl)piperidin-3-ol

The titled compound (81.3 mg) was prepared in accordance with the same procedures as in Example 7, using Intermediates A6-3 and C3-1. (Yield: 92.3%) 1H NMR (CDCl₃, 400 MHz) δ 8.42 (d, 1H), 8.28 (s, 1H), 8.13 (s, 1H), 8.08 (s, 1H), 7.86 (s, 1H), 7.60-7.53 (m, 1H), 7.44-7.34 (m, 2.2H), 7.23-7.19 (m, 0.6H), 7.08-7.04 (m, 0.2H), 6.91 (t, 1H), 6.82 (d, 1H), 4.02-3.94 (m, 1H), 3.79 (s, 3H), 3.46-3.43 (m, 2H), 3.33-3.19 (m, 2H), 2.34-2.16 (m, 1H), 1.99-1.91 (m, 1H), 1.90-1.79 (m, 1H), 1.76-1.66 (m, 2H) MS m/z=579.9 (M+H)+

### Example 223: (3S)-1-(2-((6-(2,4-difluoro-6-methoxyphenyl)-5-nitropyridin-2-yl)amino)-5-(1-(2,2-difluoroethyl)-1H-pyrazol-4-yl)pyridin-4-yl)piperidin-3-ol

The titled compound (15.0 mg) was prepared in accordance with the same procedures as in Example 7, using Intermediates A2-10 and C3-2. (Yield: 17.9%) 1H NMR (CDCl₃, 400 MHz) δ 8.46 (d, 1H), 8.18 (s, 1H), 7.90-7.81 (m, 2H), 7.81-7.70 (m, 1H), 7.63-7.51 (m, 1H), 7.45 (s, 1H), 6.69 (t, 1H), 6.58 (d, 1H), 6.33-6.00 (m, 1H), 4.65-4.46 (m, 2H), 3.91-3.79 (m, 1H), 3.79 (s, 3H), 3.26-3.16 (m, 1H), 3.05-2.90 (m, 1H), 2.78-2.58 (m, 2H), 2.00-1.89 (m, 1H), 1.87-1.73 (m, 1H), 1.56-1.43 (m, 2H) MS m/z=587.9 (M+H)+

### Example 224: (3S)-1-(2-((6-(2,4-difluoro-6-methoxyphenyl)-5-nitropyridin-2-yl)amino)-5-(1-(tetrahydro-2H-pyran-4-yl)-1H-pyrazol-4-yl)pyridin-4-yl)piperidin-3-ol

The titled compound (10.0 mg) was prepared in accordance with the same procedures as in Example 7, using Intermediates A2-1 and C3-2. (Yield: 11.6%) 1H NMR (CDCl₃, 400 MHz) δ 8.44 (d, 1H), 8.17 (s, 1H), 7.84 (s, 2H), 7.75 (s, 1H), 7.63-7.52 (m, 1H), 7.40 (s, 1H), 6.69 (t, 1H), 6.58 (d, 1H), 4.50-4.36 (m, 1H), 4.22-4.10 (m, 2H), 3.91-3.80 (m, 1H), 3.80 (s, 3H), 3.67-3.54 (m, 2H), 3.28-3.15 (m, 1H), 3.03-2.90 (m, 1H), 2.81-2.61 (m, 2H), 2.26-2.05 (m, 4H), 1.97-1.87 (m, 1H), 1.83-1.74 (m, 1H), 1.55-1.48 (m, 2H) MS m/z=607.9 (M+H)+

### Example 225: (3S)-1-(2-((6-(2,4-difluoro-6-methoxyphenyl)-5-nitropyridin-2-yl)amino)-5-((tetrahydro-2H-pyran-4-yl)ethynyl)pyridin-4-yl)piperidin-3-ol

The titled compound (8.0 mg) was prepared in accordance with the same procedures as in Example 7, using Intermediates A6-2 and C3-2. (Yield: 9.9%) 1H NMR (CDCl₃, 400 MHz) δ 8.44 (d, 1H), 8.22 (s, 1H), 7.69 (s, 1H), 7.53-7.46 (m, 1H), 7.35-7.30 (m, 1H), 6.66 (t, 1H), 6.57 (d, 1H), 4.03-3.90 (m, 3H), 3.81 (s, 3H), 3.61-3.51 (m, 2H), 3.50-3.42 (m, 1H), 3.37-3.30 (m, 1H), 3.30-3.23 (m, 1H), 3.20-3.12 (m, 1H), 2.97-2.87 (m, 1H), 2.14-2.05 (m, 1H), 2.01-1.91 (m, 2H), 1.91-1.76 (m, 3H), 1.73-1.63 (m, 2H) MS m/z=565.9 (M+H)+

### Example 226: (3S)-1-(2-((5-amino-6-(2,4-difluoro-6-methoxyphenyl)pyridin-2-yl)amino)-5-(1-(2,2-difluoroethyl)-1H-pyrazol-4-yl)pyridin-4-yl)piperidin-3-ol

The titled compound (10.2 mg) was prepared in accordance with the same procedures as in Step 2 of Example 150, using (3S)-1-(2-((6-(2,4-difluoro-6-methoxyphenyl)-5-nitropyridin-2-yl)amino)-5-(1-(2,2-difluoroethyl)-1H-pyrazol-4-yl)pyridin-4-yl)piperidin-3-ol (Example 223). (Yield: 69.2%) 1H NMR (CD₃OD, 400 MHz) δ 8.05 (s, 1H), 7.95 (s, 1H), 7.90 (s, 1H), 7.33-7.20 (m, 3H), 6.87 (d, 1H), 6.77 (t, 1H), 6.40-6.04 (m, 1H), 4.68-4.55 (m, 2H), 3.88 (s, 3H), 3.78-3.69 (m, 1H), 3.32-3.25 (m, 1H), 3.10-2.99 (m, 1H), 2.64-2.51 (m, 1H), 2.49-2.39 (m, 1H), 2.02-1.92 (m, 1H), 1.80-1.69 (m, 1H), 1.63-1.51 (m, 1H), 1.43-1.31 (m, 2H) MS m/z=557.9 (M+H)+

### Example 227: (3S)-1-(2-((5-amino-6-(2,4-difluoro-6-methoxyphenyl)pyridin-2-yl)amino)-5-(1-(tetrahydro-2H-pyran-4-yl)-1H-pyrazol-4-yl)pyridin-4-yl)piperidin-3-ol

The titled compound (5.5 mg) was prepared in accordance with the same procedures as in Step 2 of Example 150, using (3S)-1-(2-((6-(2,4-difluoro-6-methoxyphenyl)-5-nitropyridin-2-yl)amino)-5-(1-(tetrahydro-2H-pyran-4-yl)-1H-pyrazol-4-yl)pyridin-4-yl)piperidin-3-ol (Example 224). (Yield: 38.5%) 1H NMR (CD₃OD, 400 MHz) δ 8.07 (s, 1H), 7.96 (s, 1H), 7.84 (s, 1H), 7.33-7.17 (m, 3H), 6.87 (d, 1H), 6.77 (t, 1H), 4.53-4.40 (m, 1H), 4.16-4.04 (m, 2H), 3.88 (s, 3H), 3.77-3.68 (m, 1H), 3.65-3.56 (m, 2H), 3.32-3.24 (m, 1H), 3.10-2.98 (m, 1H), 2.66-2.54 (m, 1H), 2.49-2.38 (m, 1H), 2.18-2.04 (m, 4H), 2.03-1.93 (m, 1H), 1.81-1.71 (m, 1H), 1.64-1.52 (m, 1H), 1.43-1.33 (m, 1H) MS m/z=578.0 (M+H)+

### Example 228: (3S)-1-(2-((5-amino-6-(2,4-difluoro-6-methoxyphenyl)pyridin-2-yl)amino)-5-(2-(tetrahydro-2H-pyran-4-yl)ethyl)pyridin-4-yl)piperidin-3-ol

The titled compound (2.7 mg) was prepared in accordance with the same procedures as in Step 2 of Example 150, using (3S)-1-(2-((6-(2,4-difluoro-6-methoxyphenyl)-5-nitropyridin-2-yl)amino)-5-((tetrahydro-2H-pyran-4-yl)ethynyl)pyridin-4-yl)piperidin-3-ol (Example 225). (Yield: 31.2%) 1H NMR (CD₃OD, 400 MHz) δ 7.85 (s, 1H), 7.33-7.25 (m, 1H), 7.23-7.12 (m, 2H), 6.86 (d, 1H), 6.76 (t, 1H), 3.99-3.89 (m, 2H), 3.87 (s, 3H), 3.83-3.73 (m, 1H), 3.46 (t, 2H), 3.31-3.24 (m, 1H), 3.12-3.02 (m, 1H), 2.71-2.64 (m, 1H), 2.64-2.58 (m, 2H), 2.57-2.48 (m, 1H), 2.11-1.99 (m, 1H), 1.93-1.82 (m, 1H), 1.76-1.66 (m, 3H), 1.65-1.57 (m, 2H), 1.56-1.48 (m, 1H), 1.48-1.38 (m, 1H), 1.37-1.25 (m, 2H) MS m/z=540.0 (M+H)+

### Example 229: (S)-N-(4-(3-aminopiperidin-1-yl)-5-(1-(2-methoxy-2-methylpropyl)-1H-pyrazol-4-yl)pyridin-2-yl)-2-(2-fluoro-6-methoxyphenyl)pyrimidin-4-amine

The titled compound (10.8 mg) was prepared in accordance with the same procedures as in Example 52, using Intermediates A3-21 and C1-1. (Yield: 27.0%) 1H NMR (CDCl₃, 400 MHz) δ 8.60 (d, 1H), 8.16 (s, 1H), 7.96-7.85 (m, 1H), 7.83-7.72 (m, 2H), 7.50-7.42 (m, 1H), 7.40 (s, 1H), 7.28-7.23 (m, 1H), 6.86-6.73 (m, 2H), 4.22 (s, 2H), 3.85 (s, 3H), 3.35-3.30 (m, 1H), 3.30 (s, 3H), 3.23-3.14 (m, 1H), 2.98-2.85 (m, 1H), 2.57-2.46 (m, 1H), 2.35-2.24 (m, 1H), 2.00-1.90 (m, 1H), 1.75-1.56 (m, 2H), 1.23 (s, 6H), 1.17-1.09 (m, 1H) MS m/z=547.0 (M+H)+

### Example 230: (S)-4-(3-aminopiperidin-1-yl)-N-(2-(2-fluoro-6-methoxyphenyl)pyrimidin-4-yl)-6′-(2-methoxyethoxy)-[3,3′-bipyridin]-6-amine

The titled compound (7.5 mg) was prepared in accordance with the same procedures as in Example 52, using Intermediates A3-22 and C1-1. (Yield: 27.4%) 1H NMR (CDCl₃, 400 MHz) δ 8.64 (d, 1H), 8.32 (s, 1H), 8.01 (s, 1H), 7.94 (s, 1H), 7.85 (d, 1H), 7.48-7.40 (m, 1H), 7.40-7.31 (m, 2H), 6.92 (d, 1H), 6.85-6.77 (m, 2H), 4.58-4.48 (m, 2H), 3.87 (d, 3H), 3.82-3.77 (m, 2H), 3.53-3.44 (m, 3H), 3.28-3.17 (m, 1H), 3.16-3.05 (m, 1H), 2.86-2.73 (m, 1H), 2.59-2.47 (m, 1H), 2.38-2.24 (m, 1H), 1.96-1.83 (m, 1H), 1.64-1.53 (m, 1H), 1.53-1.38 (m, 1H), 1.19-1.06 (m, 2H) MS m/z=545.9 (M+H)+

### Example 231: (S)-(3,3-difluoroazetidin-1-yl)(4-(4-((5-(1-(difluoromethyl)-1H-pyrazol-4-yl)-4-(3-hydroxypiperidin-1-yl)pyridin-2-yl)amino)pyrimidin-2-yl)-3-fluoro-5-methoxyphenyl)methanone

### Step 1: methyl (S)-4-(4-((5-(1-(difluoromethyl)-1H-pyrazol-4-yl)-4-(3-hydroxypiperidin-1-yl)pyridin-2-yl)amino)pyrimidin-2-yl)-3-fluoro-5-methoxybenzoate

The titled compound (212.9 mg) was prepared in accordance with the same procedures as in Example 7, using Intermediates A2-8 and C1-10. (Yield: 37.5%) 1H NMR (CDCl₃, 400 MHz) δ 8.63 (d, 1H), 8.44 (d, 2H), 8.18-8.12 (m, 2H), 8.01 (s, 1H), 7.73 (s, 1H), 7.42-7.40 (m, 0.2H), 7.27-7.23 (m, 0.6H), 7.13-7.08 (m, 0.2H), 4.00 (s, 3H), 3.91 (s, 3H), 3.26-3.16 (m, 1H), 3.04-2.94 (m, 1H), 2.74-2.61 (m, 2H), 1.99-1.90 (m, 1H), 1.84-1.73 (m, 2H), 1.59-1.41 (m, 2H)

### Step 2: (S)-4-(4-((5-(1-(difluoromethyl)-1H-pyrazol-4-yl)-4-(3-hydroxypiperidin-1-yl)pyridin-2-yl)amino)pyrimidin-2-yl)-3-fluoro-5-methoxybenzoic acid

Methyl (S)-4-(4-((5-(1-(difluoromethyl)-1H-pyrazol-4-yl)-4-(3-hydroxypiperidin-1-yl)pyridin-2-yl)amino)pyrimidin-2-yl)-3-fluoro-5-methoxybenzoate (212.0 mg, 0.37 mmol) was dissolved in methanol (2.0 ml) and then 1.0 N sodium hydroxide solution (1.0 mL) was added dropwise thereto. Then, the mixed solution was stirred at 60 °C for 3 hours. After completion of the reaction, the reaction mixture was cooled to room temperature and then neutralized with 1.0 N hydrochloric acid solution. The aqueous layer was extracted with dichloromethane. The extracted organic layer was dried over anhydrous magnesium sulfate and then filtered. The filtrate was concentrated and then dried to prepare the titled compound (200.0 mg), without further purification. (Yield: 96.7%) 1H NMR (CD₃OD, 400 MHz) δ 8.84 (d, 1H), 8.45 (s, 1H), 8.30 (d, 2H), 8.10 (s, 1H), 8.03 (s, 1H), 6.85 (d, 2H), 4.05 (s, 3H), 3.86-3.78 (m, 1H), 3.56-3.46 (m, 1H), 3.18-3.07 (m, 1H), 2.02-1.86 (m, 2H), 1.68-1.50 (m, 2H)

### Step 3: (S)-(3,3-difluoroazetidin-1-yl)(4-(4-((5-(1-(difluoromethyl)-1H-pyrazol-4-yl)-4-(3-hydroxypiperidin-1-yl)pyridin-2-yl)amino)pyrimidin-2-yl)-3-fluoro-5-methoxyphenyl)methanone

((S)-4-(4-((5-(1-(Difluoromethyl)-1H-pyrazol-4-yl)-4-(3-hydroxypiperidin-1-yl)pyridin-2-yl)amino)pyrimidin-2-yl)-3-fluoro-5-methoxybenzoic acid (30.0 mg, 0.05 mmol) was dissolved in anhydrous dimethylformamide (1.0 mL) and then 1-[bis(dimethylamino)methylene]-1H-1,2,3-triazolo[4,5-b]pyridinium-3-oxide hexafluorophosphate (HATU, 41.0 mg, 0.11 mmol) and diisopropylethylamine (28.2 uL, 0.16 mmol) were added dropwise thereto. The reaction mixture was stirred at room temperature for 15 minutes and then 3,3-difluoroazetidine hydrochloride (14.0 mg, 0.11 mmol) was added dropwise thereto. The reaction mixture was stirred at 80 °C for 4 hours. Water was added to the reaction mixture and then the aqueous layer was extracted with ethyl acetate. The extracted organic layer was dried over anhydrous magnesium sulfate and then filtered. The filtrate was concentrated and then purified by silica gel column chromatography to prepare the titled compound (6.0 mg). (Yield: 17.6%) 1H NMR (CDCl₃, 400 MHz) δ 8.67-8.58 (m, 1H), 8.19 (s, 1H), 8.01 (s, 1H), 7.54-7.46 (m, 1H), 7.43-7.40 (m, 0.2H), 7.35-7.30 (m, 1H), 7.27-7.24 (m, 0.6H), 7.21-7.16 (m, 1H), 7.13-7.10 (m, 0.2H), 7.06 (d, 1H), 4.68-4.49 (m, 4H), 3.93-3.88 (m, 3H), 3.88-3.82 (m, 1H), 3.35-3.22 (m, 1H), 3.10-3.00 (m, 1H), 2.77-2.67 (m, 1H), 2.09-2.03 (m, 1H), 2.01-1.94 (m, 1H), 1.84-1.75 (m, 2H) MS m/z=630.9 (M+H)+

### Example 232: (4-(4-((5-(1-(difluoromethyl)-1H-pyrazol-4-yl)-4-((S)-3-hydroxypiperidin-1-yl)pyridin-2-yl)amino)pyrimidin-2-yl)-3-fluoro-5-methoxyphenyl)((S)-3-fluoropyrrolidin-1-yl)methanone

The titled compound (10.0 mg) was prepared in accordance with the same procedures as in Step 3 of Example 231, changing the amine reagent to (S)-3-fluoropyrrolidine hydrochloride. (Yield: 29.6%) 1H NMR (CDCl₃, 400 MHz) δ 8.67-8.58 (m, 1H), 8.17 (s, 1H), 8.10-8.02 (m, 1H), 7.98 (s, 1H), 7.51-7.42 (m, 1H), 7.43-7.40 (m, 0.2H), 7.39-7.33 (m, 1H), 7.26-7.25 (m, 0.6H), 7.12-7.09 (m, 0.2H), 7.05-6.98 (m, 2H), 5.48-5.17 (m, 1H), 4.98-4.84 (m, 1H), 3.88 (s, 3H), 3.79-3.72 (m, 1H), 3.69-3.60 (m, 1H), 3.34-3.21 (m, 1H), 3.10-2.97 (m, 1H), 2.73-2.58 (m, 2H), 2.43-2.29 (m, 1H), 2.05-1.96 (m, 1H), 1.83-1.72 (m, 1H), 1.68-1.58 (m, 2H), 1.57-1.43 (m, 2H) MS m/z=626.9 (M+H)+

### Example 233: (4-(4-((5-(1-(difluoromethyl)-1H-pyrazol-4-yl)-4-((S)-3-hydroxypiperidin-1-yl)pyridin-2-yl)amino)pyrimidin-2-yl)-3-fluoro-5-methoxyphenyl)((R)-3-fluoropyrrolidin-1-yl)methanone

The titled compound (11.2 mg) was prepared in accordance with the same procedures as in Step 3 of Example 231, changing the amine reagent to (R)-3-fluoropyrrolidine hydrochloride. (Yield: 33.1%) 1H NMR (CDCl₃, 400 MHz) δ 8.65-8.56 (m, 1H), 8.19 (s, 1H), 8.13-8.05 (m, 1H), 7.99 (s, 1H), 7.53-7.43 (m, 1H), 7.42-7.39 (m, 0.2H), 7.39-7.32 (m, 1H), 7.27-7.24 (m, 0.6H), 7.13-7.09 (m, 0.2H), 7.05-6.97 (m, 2H), 6.97-5.28 (m, 1H), 4.98-4.81 (m, 1H), 3.90 (s, 3H), 3.86-3.80 (m, 1H), 3.80-3.71 (m, 1H), 3.32-3.21 (m, 1H), 3.08-2.98 (m, 1H), 2.72-2.54 (m, 2H), 2.46-2.27 (m, 1H), 2.21-2.09 (m, 1H), 2.03-1.93 (m, 2H), 1.81-1.70 (m, 2H) MS m/z=626.9 (M+H)+

### Example 234: (S)-(4-(4-((5-(1-(difluoromethyl)-1H-pyrazol-4-yl)-4-(3-hydroxypiperidin-1-yl)pyridin-2-yl)amino)pyrimidin-2-yl)-3-fluoro-5-methoxyphenyl)(3,3-difluoropyrrolidin-1-yl)methanone

The titled compound (14.0 mg) was prepared in accordance with the same procedures as in Step 3 of Example 231, changing the amine reagent to 3,3-difluoropyrrolidine hydrochloride. (Yield: 40.0%) 1H NMR (CDCl₃, 400 MHz) δ 8.67-8.58 (m, 1H), 8.18 (s, 1H), 8.11-8.03 (m, 1H), 7.99 (s, 1H), 7.48-7.33 (m, 2H), 7.27-7.24 (m, 0.6H), 7.13-7.08 (m, 0.2H), 7.03-6.97 (m, 1H), 6.97-6.91 (m, 1H), 5.00-4.77 (m, 1H), 4.09-3.98 (m, 1H), 3.98-3.92 (m, 1H), 3.92 (s, 3H), 3.88-3.80 (m, 1H), 3.80-3.72 (m, 1H), 3.34-3.24 (m, 1H), 3.10-2.99 (m, 1H), 2.76-2.56 (m, 2H), 2.53-2.37 (m, 2H), 2.05-1.93 (m, 1H), 1.91-1.83 (m, 1H), 1.82-1.71 (m, 2H) MS m/z=644.9 (M+H)+

### Example 235: (S)-1-(4-(4-(3-aminopiperidin-1-yl)-6-((2-(2-fluoro-6-methoxyphenyl)pyrimidin-4-yl)amino)pyridin-3-yl)-1H-pyrazol-1-yl)-2-methylpropan-2-ol hydrochloride

The titled compound (20.1 mg) was prepared in accordance with the same procedures as in Example 52, using Intermediates A3-23 and C1-1. (Yield: 71.8%) 1H NMR (CD₃OD, 400 MHz) δ 8.91 (d, 1H), 8.27 (s, 2H), 8.04 (s, 1H), 7.83 (q, 1H), 7.66 (d, 1H), 7.47 (s, 1H), 7.25 (d, 1H), 7.13 (t, 1H), 4.35 (s, 2H), 4.07 (s, 3H), 4.00-3.96 (m, 1H), 3.75-3.69 (m, 1H), 3.53-3.42 (m, 1H), 3.20-3.02 (m, 2H), 2.28-2.17 (m, 1H), 1.95-1.63 (m, 3H), 1.32 (d, 6H) MS m/z=533.0 (M+H)+

### Example 236: (S)-N-(4-(3-aminopiperidin-1-yl)-5-(1-(3-(dimethylamino)propyl)-1H-pyrazol-4-yl)pyridin-2-yl)-2-(2-fluoro-6-methoxyphenyl)pyrimidin-4-amine hydrochloride

The titled compound (27.8 mg) was prepared in accordance with the same procedures as in Example 52, using Intermediates A3-24 and C1-1. (Yield: 49.2%) 1H NMR (CD₃OD, 400 MHz) δ 8.93 (d, 1H), 8.32 (d, 2H), 7.98 (s, 1H), 7.84 (q, 1H), 7.64 (d, 1H), 7.48 (s, 1H), 7.26 (d, 1H), 7.14 (t, 1H), 4.49 (t, 2H), 4.10 (s, 3H), 3.93-3.91 (m, 1H), 3.55-3.40 (m, 1H), 3.31-3.24 (m, 2H), 3.23-3.07 (m, 2H), 3.00-2.89 (m, 6H), 2.47-2.36 (m, 2H), 2.28-2.19 (m, 1H), 1.94-1.85 (m, 1H), 1.85-1.67 (m, 2H) MS m/z=547.0 (M+H)+

### Example 237: (S)-N-(4-(3-aminopiperidin-1-yl)-5-(1-(azetidin-3-ylmethyl)-1H-pyrazol-4-yl)pyridin-2-yl)-2-(2-fluoro-6-methoxyphenyl)pyrimidin-4-amine hydrochloride

The titled compound (16.0 mg) was prepared in accordance with the same procedures as in Example 52, using Intermediates A3-25 and C1-1. (Yield: 49.0%) 1H NMR (CD₃OD, 400 MHz) δ 8.91 (d, 1H), 8.31-8.18 (m, 2H), 7.95-7.86 (m, 1H), 7.81 (q, 1H), 7.64 (d, 1H), 7.46 (s, 1H), 7.25 (d, 1H), 7.13 (t, 1H), 4.65-4.56 (m, 2H), 4.27-4.09 (m, 3H), 4.07 (s, 3H), 3.77-3.71 (m, 1H), 3.60-3.42 (m, 2H), 3.26-3.04 (m, 2H), 2.29-2.18 (m, 1H), 1.93-1.84 (m, 1H), 1.84-1.66 (m, 2H) MS m/z=531.0 (M+H)+

### Example 238: N-(4-((S)-3-aminopiperidin-1-yl)-5-(1-((R)-pyrrolidin-3-yl)-1H-pyrazol-4-yl)pyridin-2-yl)-2-(2-fluoro-6-methoxyphenyl)pyrimidin-4-amine hydrochloride

The titled compound (18.9 mg) was prepared in accordance with the same procedures as in Example 52, using Intermediates A3-26 and C1-1. (Yield: 75.0%) 1H NMR (CD₃OD, 400 MHz) δ 8.91 (d, 1H), 8.33 (s, 1H), 8.24 (s, 1H), 7.97 (s, 1H), 7.83 (q, 1H), 7.63 (d, 1H), 7.45 (s, 1H), 7.26 (d, 1H), 7.13 (t, 1H), 5.48-5.38 (m, 1H), 4.08 (s, 3H), 3.95-3.93 (m, 1H), 3.93-3.75 (m, 2H), 3.75-3.69 (m, 1H), 3.63-3.52 (m, 1H), 3.52-3.39 (m, 1H), 3.20-3.05 (m, 2H), 2.69-2.58 (m, 1H), 2.58-2.45 (m, 1H), 2.30-2.16 (m, 1H), 1.94-1.85 (m, 1H), 1.85-1.65 (m, 2H) MS m/z=531.0 (M+H)+

### Example 239: N-(4-((S)-3-aminopiperidin-1-yl)-5-(1-((S)-pyrrolidin-3-yl)-1H-pyrazol-4-yl)pyridin-2-yl)-2-(2-fluoro-6-methoxyphenyl)pyrimidin-4-amine hydrochloride

The titled compound (15.7 mg) was prepared in accordance with the same procedures as in Example 52, using Intermediates A3-27 and C1-1. (Yield: 69.4%) 1H NMR (CD₃OD, 400 MHz) δ 8.91 (d, 1H), 8.35 (s, 1H), 8.25 (s, 1H), 7.96 (s, 1H), 7.82 (q, 1H), 7.65 (d, 1H), 7.46 (s, 1H), 7.26 (d, 1H), 7.13 (t, 1H), 5.49-5.37 (m, 1H), 4.09 (s, 3H), 3.96-3.94 (m, 1H), 3.93-3.77 (m, 2H), 3.77-3.69 (m, 1H), 3.64-3.53 (m, 2H), 3.53-3.42 (m, 1H), 3.21-3.04 (m, 2H), 2.68-2.56 (m, 1H), 2.56-2.42 (m, 1H), 2.29-2.16 (m, 1H), 1.97-1.83 (m, 1H), 1.83-1.65 (m, 2H) MS m/z=531.0 (M+H)+

### Example 240: (S)-4-(3-aminopiperidin-1-yl)-N⁶-(2-(dimethylamino)ethyl)-N⁶-(2-(2-fluoro-6-methoxyphenyl)pyrimidin-4-yl)-[3,3′-bipyridin]-6,6′-diamine hydrochloride

The titled compound (4.9 mg) was prepared in accordance with the same procedures as in Example 52, using Intermediates A3-28 and C1-1. (Yield: 70.0%) 1H NMR (CD₃OD, 400 MHz) δ 8.87 (d, 1H), 8.35-8.15 (m, 3H), 7.81 (q, 2H), 7.45-7.29 (m, 2H), 7.24 (d, 1H), 7.09 (t, 1H), 4.08-3.98 (m, 1H), 3.97 (s, 3H), 3.96-3.86 (m, 1H), 3.74-3.67 (m, 2H), 3.67-3.59 (m, 2H), 3.12-2.99 (m, 6H), 2.99-2.90 (m, 1H), 2.27-2.14 (m, 1H), 1.99-1.89 (m, 1H), 1.89-1.72 (m, 1H), 1.68-1.54 (m, 1H) MS m/z=559.0 (M+H)+

### Example 241: (S)-4-(3-aminopiperidin-1-yl)-N-(2-(2-fluoro-6-methoxyphenyl)pyrimidin-4-yl)-6′-(piperazin-1-yl)-[3,3′-bipyridin]-6-amine hydrochloride

The titled compound (20.9 mg) was prepared in accordance with the same procedures as in Example 52, using Intermediates A3-29 and C1-1. (Yield: 80.2%) 1H NMR (CD₃OD, 400 MHz) δ 8.87 (d, 1H), 8.41-8.33 (m, 2H), 8.33 (s, 1H), 7.79-7.60 (m, 3H), 7.48 (s, 1H), 7.24 (d, 1H), 7.11 (t, 1H), 4.26-4.10 (m, 4H), 4.05 (s, 3H), 3.97-3.88 (m, 1H), 3.73-3.64 (m, 1H), 3.64-3.56 (m, 4H), 3.46-3.36 (m, 1H), 3.17-3.08 (m, 1H), 3.08-2.97 (m, 1H), 2.27-2.15 (m, 1H), 2.00-1.88 (m, 1H), 1.88-1.74 (m, 1H), 1.70-1.60 (m, 1H) MS m/z=556.0 (M+H)+

### Example 242: (S)-(2-(4-((4-(3-aminopiperidin-1-yl)-5-(1-(difluoromethyl)-1H-pyrazol-4-yl)pyridin-2-yl)amino)pyrimidin-2-yl)-3-fluorophenyl)methanol hydrochloride

The titled compound (11.9 mg) was prepared in accordance with the same procedures as in Example 52, using Intermediates A3-2 and C1-11. (Yield: 62.5%) 1H NMR (CD₃OD, 400 MHz) δ 8.90 (d, 1H), 8.57 (s, 1H), 8.09 (s, 1H), 8.05 (s, 1H), 7.80-7.76 (m, 0.2H), 7.76-7.68 (m, 1H), 7.64-7.62 (m, 0.6H), 7.59 (d, 1H), 7.53-7.50 (m, 1H), 7.49-7.47 (m, 0.2H), 7.43 (t, 1H), 7.29 (s, 1H), 4.73 (s, 2H), 3.98-3.85 (m, 1H), 3.66-3.55 (m, 1H), 3.50-3.42 (m, 1H), 3.16-3.04 (m, 1H), 3.04-2.95 (m, 1H), 2.29-2.15 (m, 1H), 1.93-1.81 (m, 1H), 1.81-1.59 (m, 2H) MS m/z=510.9 (M+H)+

### Example 243: (S)-(2-(4-((4-(3-aminopiperidin-1-yl)-5-((1-(difluoromethyl)-1H-pyrazol-4-yl)ethynyl)pyridin-2-yl)amino)pyrimidin-2-yl)-3-fluorophenyl)methanol hydrochloride

The titled compound (13.3 mg) was prepared in accordance with the same procedures as in Example 52, using Intermediates A7-3 and C1-11. (Yield: 60.3%) 1H NMR (CD₃OD, 400 MHz) δ 8.86 (d, 1H), 8.52 (s, 1H), 8.28 (s, 1H), 7.99 (s, 1H), 7.77-7.68 (m, 1H), 7.65-7.59 (m, 1H), 7.58-7.56 (m, 0.6H), 7.54 (d, 1H), 7.44-7.35 (m, 1H), 7.15 (s, 1H), 4.76 (s, 2H), 4.49-4.39 (m, 1H), 4.38-4.29 (m, 1H), 3.82-3.62 (m, 1H), 3.51-3.42 (m, 2H), 2.33-2.23 (m, 1H), 2.13-2.02 (m, 1H), 1.98-1.75 (m, 2H) MS m/z=534.9 (M+H)+

### Example 244: (S)-N-(4-(3-aminopiperidin-1-yl)-5-(1-(1-methoxy-2-methylpropan-2-yl)-1H-pyrazol-4-yl)pyridin-2-yl)-2-(2-fluoro-6-methoxyphenyl)pyrimidin-4-amine hydrochloride

The titled compound (9.7 mg) was prepared in accordance with the same procedures as in Example 52, using Intermediates A3-30 and C1-1. (Yield: 52.3%) 1H NMR (CD₃OD, 400 MHz) δ 8.87 (d, 1H), 8.22 (s, 2H), 7.89 (s, 1H), 7.79 (q, 1H), 7.61 (d, 1H), 7.37 (s, 1H), 7.24 (d, 1H), 7.11 (t, 1H), 4.03 (s, 3H), 3.97-3.92 (m, 1H), 3.73 (s, 3H), 3.55 (s, 2H), 3.47-3.41 (m, 1H), 3.15-3.03 (m, 1H), 3.03-2.92 (m, 1H), 2.28-2.18 (m, 1H), 1.90-1.80 (m, 1H), 1.80-1.74 (m, 1H), 1.74-1.69 (m, 1H), 1.68 (s, 6H) MS m/z=547.0 (M+H)+

### Example 245: (S)-N-(4-(3-aminopiperidin-1-yl)-5-(1-((3,3-difluorocyclobutyl)meth yl)-1H-pyrazol-4-yl)pyridin-2-yl)-2-(2-fluoro-6-methoxyphenyl)pyrimidin-4-amine hydrochloride

The titled compound (15.9 mg) was prepared in accordance with the same procedures as in Example 52, using Intermediates A3-31 and C1-1. (Yield: 80.2%) 1H NMR (CD₃OD, 400 MHz) δ 8.87 (d, 1H), 8.21 (s, 2H), 7.89 (s, 1H), 7.78 (q, 1H), 7.61-7.51 (m, 1H), 7.35 (s, 1H), 7.22 (d, 1H), 7.11 (t, 1H), 4.46 (d, 2H), 4.03 (s, 3H), 4.01-3.97 (m, 1H), 3.97-3.90 (m, 1H), 3.72-3.66 (m, 1H), 3.66-3.58 (m, 1H), 3.51-3.44 (m, 1H), 3.16-3.07 (m, 1H), 3.07-2.95 (m, 1H), 2.80-2.63 (m, 3H), 2.57-2.42 (m, 2H), 2.26-2.17 (m, 1H), 1.89-1.80 (m, 1H), 1.78-1.63 (m, 2H) MS m/z=565.0 (M+H)+

### Example 246: (S)-N-(4-(3-aminopiperidin-1-yl)-5-(1-(2-(dimethylamino)ethyl)-1H-pyrazol-4-yl)pyridin-2-yl)-2-(2-fluoro-6-methoxyphenyl)pyrimidin-4-amine hydrochloride

The titled compound (4.2 mg) was prepared in accordance with the same procedures as in Example 52, using Intermediates A3-32 and C1-1. (Yield: 85.2%) 1H NMR (CD₃OD, 400 MHz) δ 8.83 (d, 1H), 8.32 (s, 1H), 8.24 (s, 1H), 7.99 (s, 1H), 7.74 (q, 1H), 7.62 (s, 1H), 7.34 (s, 1H), 7.21 (d, 1H), 7.07 (t, 1H), 4.79-4.67 (m, 2H), 3.98 (s, 3H), 3.85-3.74 (m, 2H), 3.66-3.57 (m, 1H), 3.54-3.40 (m, 1H), 3.09-3.01 (m, 6H), 3.01-2.92 (m, 2H), 2.29-2.16 (m, 1H), 1.97-1.72 (m, 2H), 1.72-1.55 (m, 1H) MS m/z=532.0 (M+H)+

### Example 247: (S)-N-(4-(3-aminopiperidin-1-yl)-5-(1-(fluoromethyl)-1H-pyrazol-4-yl)pyridin-2-yl)-2-(2-fluoro-6-methoxyphenyl)pyrimidin-4-amine hydrochloride

The titled compound (4.7 mg) was prepared in accordance with the same procedures as in Example 52, using Intermediates A3-33 and C1-1. (Yield: 42.1%) 1H NMR (CDCl₃, 400 MHz) δ 8.86 (d, 1H), 8.46 (s, 1H), 8.26 (s, 1H), 8.06 (s, 1H), 7.78 (q, 1H), 7.65 (s, 1H), 7.40 (s, 1H), 7.24 (d, 1H), 7.11 (t, 1H), 6.31 (s, 1H), 6.18 (s, 1H), 4.04 (s, 3H), 3.94-3.84 (m, 1H), 3.72 (s, 2H), 3.66-3.57 (m, 1H), 3.49-3.39 (m, 1H), 3.11-2.96 (m, 2H), 2.28-2.16 (m, 1H), 1.92-1.72 (m, 2H), 1.72-1.59 (m, 1H) MS m/z=492.9 (M+H)+

### Example 248: (S)-4-(3-aminopiperidin-1-yl)-N-(2-(2-fluoro-6-methoxyphenyl)pyrimidin-4-yl)-6′-(piperidin-4-yl)-[3,3′-bipyridin]-6-amine hydrochloride

The titled compound (4.7 mg) was prepared in accordance with the same procedures as in Example 52, using Intermediates A3-34 and C1-1. (Yield: 29.1%) 1H NMR (CD₃OD, 400 MHz) δ 9.19 (s, 1H), 8.94 (s, 1H), 8.85 (d, 1H), 8.46 (s, 1H), 8.28 (s, 1H), 7.92-7.78 (m, 1H), 7.78 (q, 1H), 7.55 (s, 1H), 7.23 (d, 1H), 7.10 (t, 1H), 4.08 (s, 3H), 3.85-3.74 (m, 2H), 3.72-3.65 (m, 3H), 3.65-3.58 (m, 2H), 3.31-3.26 (m, 1H), 3.14-2.97 (m, 2H), 2.51-2.38 (m, 2H), 2.34-2.13 (m, 3H), 1.94-1.82 (m, 1H), 1.82-1.70 (m, 1H), 1.70-1.59 (m, 1H) MS m/z=556.0 (M+H)+

### Example 249: (S)-4-(3-aminopiperidin-1-yl)-6′-(2-(dimethylamino)ethoxy)-N-(2-(2-fluoro-6-methoxyphenyl)pyrimidin-4-yl)-[3,3′-bipyridin]-6-amine hydrochloride

The titled compound (5.2 mg) was prepared in accordance with the same procedures as in Example 52, using Intermediates A3-35 and C1-1. (Yield: 31.7%) 1H NMR (CD₃OD, 400 MHz) δ 8.88 (d, 1H), 8.47 (s, 1H), 8.19 (s, 1H), 8.08 (d, 1H), 7.78 (q, 1H), 7.65 (d, 1H), 7.41 (s, 1H), 7.22 (d, 1H), 7.16 (d, 1H), 7.10 (t, 1H), 4.84 (s, 2H), 4.05 (s, 3H), 3.96-3.89 (m, 1H), 3.79-3.74 (m, 1H), 3.64-3.59 (m, 1H), 3.59-3.55 (m, 1H), 3.45-3.37 (m, 1H), 3.15-3.07 (m, 1H), 3.07-2.97 (m, 6H), 2.23-2.13 (m, 1H), 1.88-1.76 (m, 1H), 1.73-1.57 (m, 2H) MS m/z=560.0 (M+H)+

### Example 250: (S)-1-(4′-(3-aminopiperidin-1-yl)-6′-((2-(2-fluoro-6-methoxyphenyl)pyrimidin-4-yl)amino)-[3,3′-bipyridin]-6-yl)-3-ethylurea hydrochloride

The titled compound (13.9 mg) was prepared in accordance with the same procedures as in Example 52, using Intermediates A3-36 and C1-1. (Yield: 33.3%) 1H NMR (CD₃OD, 400 MHz) δ 8.91 (d, 1H), 8.63-8.51 (m, 2H), 8.36 (s, 1H), 7.81 (q, 2H), 7.61 (d, 1H), 7.49 (s, 1H), 7.24 (d, 1H), 7.11 (t, 1H), 4.08 (s, 3H), 3.92-3.83 (m, 1H), 3.74-3.65 (m, 1H), 3.65-3.56 (m, 1H), 3.44 (q, 2H), 3.14-3.01 (m, 2H), 2.26-2.14 (m, 1H), 1.94-1.82 (m, 1H), 1.82-1.60 (m, 2H), 1.28 (t, 3H) MS m/z=558.0 (M+H)+

### Example 251: (4-(4-((4-((S)-3-aminopiperidin-1-yl)-5-(1-(difluoromethyl)-1H-pyrazol-4-yl)pyridin-2-yl)amino)pyrimidin-2-yl)-3-fluoro-5-methoxyphenyl)((R)-3-fluoropyrrolidin-1-yl)methanone

The titled compound (17.3 mg) was prepared in accordance with the same procedures as in Step 3 of Example 186, changing the amine reagent to (R)-3-fluoropyrrolidine hydrochloride. (Yield: 31.7%) 1H NMR (CDCl₃, 400 MHz) δ 8.63 (d, 1H), 8.28 (s, 1H), 8.17 (s, 2H), 8.02 (s, 1H), 7.57 (s, 1H), 7.44-7.40 (m, 0.2H), 7.37 (s, 1H), 7.28-7.25 (m, 0.6H), 7.13-7.09 (m, 0.2H), 7.05-6.90 (m, 2H), 5.49-5.15 (m, 1H), 4.07-3.89 (m, 1H), 3.88 (s, 3H), 3.82-3.69 (m, 2H), 3.69-3.57 (m, 2H), 3.30-3.19 (m, 1H), 3.19-3.08 (m, 1H), 2.98-2.83 (m, 1H), 2.59-2.45 (m, 1H), 2.41-2.27 (m, 2H), 2.01-1.90 (m, 1H), 1.74-1.63 (m, 1H), 1.63-1.43 (m, 2H), 1.22-1.09 (m, 1H) MS m/z=625.9 (M+H)+

### Example 252: N-(4-((2R,5S)-5-amino-2-methylpiperidin-1-yl)-5-(1-(difluoromethyl)-1H-pyrazol-4-yl)pyridin-2-yl)-2-(2-fluoro-6-methoxyphenyl)pyrimidin-4-amine

The titled compound (3.4 mg) was prepared in accordance with the same procedures as in Example 52, using Intermediates B3-36 and C1-1. (Yield: 40.1%) 1H NMR (CDCl₃, 400 MHz) δ 8.65 (d, 1H), 8.15-8.07 (m, 2H), 8.00 (s, 1H), 7.74 (s, 1H), 7.51-7.42 (m, 1H), 7.41-7.40 (m, 0.2H), 7.39-7.33 (m, 1H), 7.33-7.30 (m, 1H), 7.27-7.24 (m, 0.6H), 7.12-7.09 (m, 0.2H), 6.89-6.76 (m, 2H), 3.89 (s, 3H), 3.58-3.46 (m, 1H), 3.12-2.99 (m, 1H), 2.99-2.85 (m, 1H), 2.80-2.68 (m, 1H), 2.06-2.00 (m, 2H), 1.85-1.67 (m, 1H), 1.50 (s, 3H) MS m/z=524.9 (M+H)+

### Example 253: N-(4-((3R,4S)-3-amino-4-methoxypiperidin-1-yl)-5-(1-(difluoromethyl)-1H-pyrazol-4-yl)pyridin-2-yl)-2-(2-fluoro-6-methoxyphenyl)pyrimidin-4-amine

The titled compound (5.4 mg) was prepared in accordance with the same procedures as in Example 52, using Intermediates B3-37 and C1-1. (Yield: 34.1%) 1H NMR (CDCl₃, 400 MHz) δ 8.66 (d, 1H), 8.19-8.08 (m, 2H), 8.03 (s, 1H), 7.62-7.51 (m, 1H), 7.51 (d, 1H), 7.42-7.32 (m, 2.2H), 7.27-7.23 (m, 0.6H), 7.13-7.09 (m, 0.2H), 6.89-6.76 (m, 2H), 3.90 (s, 3H), 3.46-3.40 (m, 1H), 3.40 (s, 3H), 3.06-2.98 (m, 1H), 2.98-2.87 (m, 3H), 2.87-2.78 (m, 1H), 1.97-1.87 (m, 1H), 1.65-1.56 (m, 2H) MS m/z=540.9 (M+H)+

### Example 254: N-(4-((3R,4R)-3-amino-4-methoxypiperidin-1-yl)-5-(1-(difluoromethyl)-1H-pyrazol-4-yl)pyridin-2-yl)-2-(2-fluoro-6-methoxyphenyl)pyrimidin-4-amine

The titled compound (5.9 mg) was prepared in accordance with the same procedures as in Example 52, using Intermediates B3-38 and C1-1. (Yield: 38.1%) 1H NMR (CDCl₃, 400 MHz) δ 8.65 (d, 1H), 8.19-8.08 (m, 2H), 8.01 (s, 1H), 7.71 (s, 1H), 7.50 (d, 1H), 7.43-7.32 (m, 2.2H), 7.27-7.24 (m, 0.6H), 7.12-7.08 (m, 0.2H), 6.88-6.78 (m, 2H), 3.89 (s, 3H), 3.46 (s, 3H), 3.36-3.25 (m, 2H), 2.99-2.86 (m, 2H), 2.70-2.56 (m, 1H), 2.54-2.41 (m, 1H), 2.09-1.99 (m, 1H), 1.59-1.47 (m, 2H) MS m/z=540.9 (M+H)+

### Example 255: (S)-(4-(4-(3-aminopiperidin-1-yl)-6-((2-(2-fluoro-6-methoxyphenyl)pyrimidin-4-yl)amino)pyridin-3-yl)phenyl)(morpholino)methanone hydrochloride

The titled compound (38.2 mg) was prepared in accordance with the same procedures as in Example 52, using Intermediates A3-37 and C1-1. (Yield: 87.2%) 1H NMR (CD₃OD, 400 MHz) δ 8.77 (d, 1H), 8.12 (s, 1H), 7.71-7.65 (m 2H), 7.65-7.59 (m, 3H), 7.59-7.46 (m, 1H), 7.22 (s, 1H), 7.13 (d, 1H), 7.00 (t, 1H), 3.94 (s, 3H), 3.86-3.74 (m, 4H), 3.74-3.60 (m, 3H), 3.57-3.45 (m, 3H), 3.31-3.24 (m, 1H), 2.93 (q, 2H), 2.17-2.13 (m, 1H), 1.82-1.78 (m, 1H), 1.72-1.54 (m, 2H) MS m/z=584.9 (M+H)+

### Example 256: (S)-N-(4-(3-aminopiperidin-1-yl)-5-(4-methyl-3,4-dihydro-2H-pyrido[3,2-b][1,4]oxazin-7-yl)pyridin-2-yl)-2-(2-fluoro-6-methoxyphenyl)pyrimidin-4-amine hydrochloride

The titled compound (31.7 mg) was prepared in accordance with the same procedures as in Example 52, using Intermediates A3-38 and C1-1. (Yield: 94.5%) 1H NMR (CD₃OD, 400 MHz) δ 8.71 (d, 1H), 8.20 (s, 1H), 7.80 (s, 1H), 7.80-7.62 (m 3H), 7.28 (brs, 1H), 7.13 (d, 1H), 7.00 (t, 1H), 4.45 (t, 2H), 3.94 (s, 3H), 3.86 (t, 2H), 3.80 (d, 1H), 3.51 (d, 1H), 3.40 (s, 3H), 3.35-3.25 (m, 1H), 2.93 (t, 1H), 2.84 (t, 1H), 2.19 (d, 1H), 1.91 (d, 1H), 1.74 (q, 1H), 1.58 (q, 1H) MS m/z=543.9 (M+H)+

### Example 257: (S)-N-(4-(3-aminopiperidin-1-yl)-5-(4-methyl-3,4-dihydro-2H-benzo[b][1,4]oxazin-7-yl)pyridin-2-yl)-2-(2-fluoro-6-methoxyphenyl)pyrimidin-4-amine hydrochloride

The titled compound (16.8 mg) was prepared in accordance with the same procedures as in Example 52, using Intermediates A3-39 and C1-1. (Yield: 64.3%) 1H NMR (CD₃OD, 400 MHz) δ 8.78 (d, 1H), 7.96 (s, 1H), 7.66 (q, 1H), 7.48-7.38 (m 1H), 7.14-7.11 (m, 2H), 7.02-6.97 (m, 2H), 6.90-6.84 (m, 2H), 4.32 (t, 2H), 3.94 (s, 3H), 3.91 (d, 1H), 3.64 (d, 1H), 3.37 (t, 2H), 3.37-3.20 (m, 1H), 2.97 (s, 3H), 3.01-2.90 (m, 2H), 2.25-2.15 (m, 1H), 1.85-1.75 (m, 1H), 1.75-1.50 (m, 2H) MS m/z=543.0 (M+H)+

### Example 258: (S)-6-(4-(3-aminopiperidin-1-yl)-6-((2-(2-fluoro-6-methoxyphenyl)pyrimidin-4-yl)amino)pyridin-3-yl)-1,4-dihydro-2H-benzo[d][1,3]oxazin-2-one hydrochloride

The titled compound (16.9 mg) was prepared in accordance with the same procedures as in Example 52, using Intermediates A3-40 and C1-1. (Yield: 63.6%) 1H NMR (CD₃OD, 400 MHz) δ 8.76 (d, 1H), 8.04 (s, 1H), 7.63 (q, 1H), 7.55-7.45 (m 2H), 7.42 (s, 1H), 7.16 (s, 1H), 7.11 (d, 1H), 7.07 (d, 1H), 6.98 (t, 1H), 5.42 (s, 2H), 3.93 (s, 3H), 3.82 (d, 1H), 3.55 (d, 1H), 3.34-3.32 (m, 1H), 3.04-2.85 (m, 2H), 2.25-2.10 (m, 1H), 1.85-1.70 (m, 1H), 1.70-1.50 (m, 2H) MS m/z=542.9 (M+H)+

### Example 259: (S)-1-(4-(4-(3-aminopiperidin-1-yl)-6-((2-(2-fluoro-6-methoxyphenyl)pyrimidin-4-yl)amino)pyridin-3-yl)phenyl)pyrrolidin-2-one hydrochloride

The titled compound (25.1 mg) was prepared in accordance with the same procedures as in Example 52, using Intermediates A3-41 and C1-1. (Yield: 42.1%) 1H NMR (CD₃OD, 400 MHz) δ 8.44 (d, 1H), 7.96 (s, 1H), 7.77-7.66 (m, 2H), 7.64-7.50 (m, 4H), 7.44 (q, 1H), 6.98 (d, 1H), 6.87 (t, 1H), 3.99 (t, 2H), 3.84 (s, 3H), 3.24 (d, 1H), 3.14 (d, 1H), 2.80-2.70 (m, 1H), 2.64 (t, 2H), 2.57 (t, 1H), 2.34 (t, 1H), 2.30-2.15 (m, 2H), 1.95-1.85 (m, 1H), 1.60-1.45 (m, 1H), 1.40-1.25 (m, 1H), 1.25-1.10 (m, 1H) MS m/z=554.9 (M+H)+

### Example 260: (S)-7-(4-(3-aminopiperidin-1-yl)-6-((2-(2-fluoro-6-methoxyphenyl)pyrimidin-4-yl)amino)pyridin-3-yl)-2H-benzo[b][1,4]oxazin-3(4H)-one hydrochloride

The titled compound (14.0 mg) was prepared in accordance with the same procedures as in Example 52, using Intermediates A3-42 and C1-1. (Yield: 65.9%) 1H NMR (CD₃OD, 400 MHz) δ 8.74 (d, 1H), 8.04 (s, 1H), 7.60 (q, 1H), 7.55-7.30 (m 1H), 7.19-7.15 (m, 2H), 7.15-7.02 (m, 3H), 6.96 (t, 1H), 4.66 (s, 2H), 3.91 (s, 3H), 3.77 (d, 1H), 3.54 (d, 1H), 2.90 (t, 2H), 2.25-2.15 (m, 1H), 2.10-2.00 (m, 1H), 1.85-1.75 (m, 1H), 1.75-1.65 (m, 11H), 1.65-1.50 (m, 1H) MS m/z=542.9 (M+H)+

### Example 261: (S)-7-(4-(3-aminopiperidin-1-yl)-6-((2-(2-fluoro-6-methoxyphenyl)pyrimidin-4-yl)amino)pyridin-3-yl)-4-methyl-2H-benzo[b][1,4]oxazin-3(4H)-one hydrochloride

The titled compound (11.3 mg) was prepared in accordance with the same procedures as in Example 52, using Intermediates A3-43 and C1-1. (Yield: 45.0%) 1H NMR (CD₃OD, 400 MHz) δ 8.75 (d, 1H), 8.06 (s, 1H), 7.61 (q, 1H), 7.58-7.40 (m, 1H), 7.40-7.25 (m, 2H), 7.25-7.20 (m, 1H), 7.15-7.05 (m, 2H), 6.97 (t, 1H), 4.71 (s, 2H), 3.92 (s, 3H), 3.80 (d, 1H), 3.53 (d, 1H), 3.43 (s, 3H), 3.00-2.85 (m, 2H), 2.25-2.15 (m, 1H), 2.10-2.00 (m, 1H), 1.85-1.75 (m, 1H), 1.75-1.50 (m, 2H) MS m/z=557.0 (M+H)+

### Example 262: (S)-6-(4-(3-aminopiperidin-1-yl)-6-((2-(2-fluoro-6-methoxyphenyl)pyrimidin-4-yl)amino)pyridin-3-yl)-8-methyl-3,4-dihydroquinolin-2(1H)-one hydrochloride

The titled compound (10.6 mg) was prepared in accordance with the same procedures as in Example 52, using Intermediates A3-44 and C1-1. (Yield: 70.8%) 1H NMR (CD₃OD, 400 MHz) δ 8.81 (d, 1H), 8.03 (s, 1H), 7.70 (q, 1H), 7.52 (d, 1H), 7.30-7.20 (m 3H), 7.16 (d, 1H), 7.03 (t, 1H), 3.98 (s, 3H), 3.98-3.90 (m, 1H), 3.80-3.70 (m, 1H), 3.65-3.55 (m, 1H), 3.10-3.00 (m, 3H), 3.00-2.90 (m, 1H), 2.63 (t, 2H), 2.35 (s, 3H), 2.25-2.15 (m, 1H), 1.85-1.70 (m, 1H), 1.70-1.55 (m, 2H) MS m/z=555.9 (M+H)+

### Example 263: (S)-6-(4-(3-aminopiperidin-1-yl)-6-((2-(2-fluoro-6-methoxyphenyl)pyrimidin-4-yl)amino)pyridin-3-yl)-1-methyl-3,4-dihydroquinolin-2(1H)-one hydrochloride

The titled compound (7.2 mg) was prepared in accordance with the same procedures as in Example 52, using Intermediates A3-45 and C1-1. (Yield: 91.7%) 1H NMR (CD₃OD, 400 MHz) δ 8.75 (d, 1H), 8.05 (s, 1H), 7.61 (q, 1H), 7.54-7.40 (m, 3H), 7.29 (d, 1H), 7.15-7.05 (m, 2H), 6.97 (t, 1H), 3.92 (s, 3H), 3.82 (d, 1H), 3.60-3.50 (m, 1H), 3.41 (s, 3H), 3.03 (t, 2H), 3.03-2.85 (m, 2H), 2.69 (t, 2H), 2.24-2.14 (m, 1H), 2.10-2.00 (m, 1H), 1.85-1.75 (m, 1H), 1.70-1.50 (m, 2H) MS m/z=556.0 (M+H)+

### Example 264: (S)-1-(2-((2-(2-fluoro-6-methoxyphenyl)pyrimidin-4-yl)amino)-5-(1-(2,2,6,6-tetramethyltetrahydro-2H-pyran-4-yl)-1H-pyrazol-4-yl)pyridin-4-yl)piperidin-3-ol

The titled compound (35.3 mg) was prepared in accordance with the same procedures as in Example 7, using Intermediates A2-20 and C1-1. (Yield: 50.4%) 1H NMR (CD₃OD, 400 MHz) δ 8.43 (d, 1H), 8.15-8.05 (m, 2H), 7.86 (s, 1H), 7.63 (s, 1H), 7.53 (d, 1H), 7.44 (q, 1H), 6.97 (d, 1H), 6.86 (t, 1H), 4.90-4.75 (m, 1H), 3.84 (s, 3H), 3.75-3.65 (m, 1H), 3.35-3.25 (m, 1H), 3.06 (d, 1H), 2.64 (t, 1H), 2.45 (t, 1H), 2.15-2.05 (m, 2H), 2.00-1.95 (m, 1H), 1.95-1.85 (m, 2H), 1.80-1.70 (m, 1H), 1.65-1.50 (m, 1H), 1.43 (s, 6H), 1.43-1.32 (m, 1H), 1.31 (s, 6H) MS m/z=602.0 (M+H)+

### Example 265: (S)-(4-(4-(3-aminopiperidin-1-yl)-6-((2-(2-fluoro-6-methoxyphenyl)pyrimidin-4-yl)amino)pyridin-3-yl)phenyl)(piperazin-1-yl)methanone hydrochloride

The titled compound (22.1 mg) was prepared in accordance with the same procedures as in Example 52, using Intermediates A3-46 and C1-1. (Yield: 79.3%) 1H NMR (CD₃OD, 400 MHz) δ 8.68 (d, 1H), 8.10 (s, 1H), 7.80-7.70 (m, 2H), 7.70-7.60 (m, 2H), 7.60-7.40 (m, 2H), 7.19 (brs, 1H), 7.07 (d, 1H), 6.95 (t, 1H), 4.10-3.75 (m, 4H), 3.90 (s, 3H), 3.70-3.55 (m, 1H), 3.50-3.30 (m, 4H), 3.30-3.20 (m, 2H), 2.95-2.75 (m, 2H), 2.20-2.10 (m, 1H), 1.85-1.75 (m, 1H), 1.75-1.60 (m, 1H), 1.60-1.45 (m, 1H) MS m/z=584.0 (M+H)+

### Example 266: (S)-N-(5-(1-(difluoromethyl)-1H-pyrazol-4-yl)-4-(3-((methylamino)methyl)piperidin-1-yl)pyridin-2-yl)-2-(2-fluoro-6-methoxyphenyl)pyrimidin-4-amine hydrochloride

The titled compound (30.5 mg) was prepared in accordance with the same procedures as in Example 52, using Intermediates B3-39 and C1-1. (Yield: 67.8%) 1H NMR (CD₃OD, 400 MHz) δ 8.78 (d, 1H), 8.48 (s, 1H), 8.16 (s, 1H), 8.06 (s, 1H), 7.77-7.45 (m, 1H), 7.68 (q, 1H), 7.47 (brs, 1H), 7.29 (s, 1H), 7.15 (d, 1H), 7.01 (t, 1H), 3.96 (s, 3H), 3.77 (d, 1H), 3.58 (d, 1H), 2.95 (d, 2H), 2.95-2.80 (m, 2H), 2.74 (s, 3H), 2.25-2.15 (m, 1H), 2.05-1.95 (m, 1H), 1.80-1.70 (m, 1H), 1.70-1.55 (m, 1H), 1.45-1.30 (m, 1H) MS m/z=539.9 (M+H)+

### Example 267: (R)-N-(5-(1-(difluoromethyl)-1H-pyrazol-4-yl)-4-(3-((methylamino)methyl)piperidin-1-yl)pyridin-2-yl)-2-(2-fluoro-6-methoxyphenyl)pyrimidin-4-amine hydrochloride

The titled compound (32.1 mg) was prepared in accordance with the same procedures as in Example 52, using Intermediates B3-40 and C1-1. (Yield: 62.9%) 1H NMR (CD₃OD, 400 MHz) δ 8.80 (d, 1H), 8.49 (s, 1H), 8.16 (s, 1H), 8.06 (s, 1H), 7.78-7.47 (m, 1H), 7.69 (q, 1H), 7.55-7.52 (m, 1H), 7.32 (s, 1H), 7.16 (d, 1H), 7.02 (t, 1H), 3.97 (s, 3H), 3.80 (d, 1H), 3.60 (d, 1H), 2.96 (d, 2H), 2.95-2.82 (m, 2H), 2.74 (s, 3H), 2.30-2.15 (m, 1H), 2.05-1.95 (m, 1H), 1.80-1.70 (m, 1H), 1.70-1.55 (m, 1H), 1.45-1.30 (m, 1H) MS m/z=539.9 (M+H)+

### Example 268: N-(4-((3R,4R)-4-amino-3-methylpiperidin-1-yl)-5-(1-(difluoromethyl)-1H-pyrazol-4-yl)pyridin-2-yl)-2-(2-fluoro-6-methoxyphenyl)pyrimidin-4-amine hydrochloride

The titled compound (9.0 mg) was prepared in accordance with the same procedures as in Example 52, using Intermediates B3-41 and C1-1. (Yield: 28.8%) 1H NMR (CD₃OD, 400 MHz) δ 8.76 (d, 1H), 8.48 (s, 1H), 8.18 (s, 1H), 8.06 (s, 1H), 7.78-7.47 (m, 1H), 7.66 (q, 1H), 7.60-7.50 (m, 1H), 7.23 (s, 1H), 7.13 (d, 1H), 7.00 (t, 1H), 3.95 (s, 3H), 3.72 (d, 1H), 3.59 (d, 1H), 3.12-2.98 (m, 2H), 2.71 (t, 1H), 2.15-2.05 (m, 1H), 1.98-1.82 (m, 1H), 1.82-1.70 (m, 1H), 1.00 (d, 3H) MS m/z=525.0 (M+H)+

### Example 269: (S)-N-(4-(3-aminopiperidin-1-yl)-5-(1-(2,2,6,6-tetramethyltetrahydro-2H-pyran-4-yl)-1H-pyrazol-4-yl)pyridin-2-yl)-2-(2-fluoro-6-methoxyphenyl)pyrimidin-4-amine hydrochloride

The titled compound (23.6 mg) was prepared in accordance with the same procedures as in Example 52, using Intermediates A3-47 and C1-1. (Yield: 54.7%) 1H NMR (CD₃OD, 400 MHz) δ 8.78 (d, 1H), 8.19 (s, 1H), 8.15 (s, 1H), 7.86 (s, 1H), 7.68 (q, 1H), 7.60-7.50 (m, 1H), 7.24 (s, 1H), 7.15 (d, 1H), 7.02 (t, 1H), 3.96 (s, 3H), 3.90 (d, 1H), 3.61 (d, 1H), 3.55-3.40 (m, 1H), 3.03 (t, 1H), 2.96 (t, 1H), 2.25-2.15 (m, 1H), 2.15-2.05 (m, 2H), 1.95-1.80 (m, 3H), 1.80-1.55 (m, 3H), 1.43 (s, 6H), 1.31 (s, 6H) MS m/z=602.0 (M+H)+

### Example 270: 6-(4-((S)-3-aminopiperidin-1-yl)-6-((2-(2-fluoro-6-methoxyphenyl)pyrimidin-4-yl)amino)pyridin-3-yl)-2-methyl-2H-benzo[b][1,4]oxazin-3(4H)-one hydrochloride

The titled compound (7.3 mg) was prepared in accordance with the same procedures as in Example 52, using Intermediates A3-48 and C1-1. (Yield: 22.2%) 1H NMR (CD₃OD, 400 MHz) δ 8.48 (d, 1H), 8.02 (s, 1H), 7.66-7.60 (m, 1H), 7.60-7.50 (m, 1H), 7.46 (q, 1H), 7.25-7.20 (m, 1H), 7.19 (s, 1H), 7.19-7.05 (m 1H), 6.99 (d, 1H), 6.88 (t, 1H), 4.71 (q, 1H), 3.84 (s, 3H), 3.55-3.40 (m, 1H), 3.30-3.20 (m, 1H), 3.20-3.10 (m, 1H), 2.75-2.65 (m, 1H), 2.65-2.55 (m, 1H), 2.15-2.00 (m, 1H), 1.75-1.60 (m, 2H), 1.57 (d, 3H), 1.55-1.40 (m, 1H) MS m/z=556.9 (M+H)+

### Example 271: (S)-N-(5-(1-(difluoromethyl)-1H-pyrazol-4-yl)-4-(3-methylpiperidin-1-yl)pyridin-2-yl)-2-(2-fluoro-6-methoxyphenyl)pyrimidin-4-amine

The titled compound (16.3 mg) was prepared in accordance with the same procedures as in Example 7, using Intermediates B3-42 and C1-1. (Yield: 20.6%) 1H NMR (CD₃OD, 400 MHz) δ 8.44 (d, 1H), 8.38 (s, 1H), 8.14 (s, 1H), 8.09 (s, 1H), 7.78 (s, 1H), 7.78-7.39 (m, 2H), 7.47 (q, 1H), 6.98 (d, 1H), 6.86 (t, 1H), 3.84 (s, 3H), 3.25-3.10 (m, 2H), 2.50-2.40 (m, 1H), 2.16 (t, 1H), 1.85-1.75 (m, 1H), 1.75-1.65 (m, 1H), 1.65-1.55 (m, 2H), 1.05-0.85 (m, 2H), 0.79 (d, 3H) MS m/z=510.9 (M+H)+

### Example 272: (S)-N-(5-(1-(difluoromethyl)-1H-pyrazol-4-yl)-4-(3-(fluoromethyl)piperidin-1-yl)pyridin-2-yl)-2-(2-fluoro-6-methoxyphenyl)pyrimidin-4-amine

The titled compound (29.6 mg) was prepared in accordance with the same procedures as in Example 7, using Intermediates B3-43 and C1-1. (Yield: 36.1%) 1H NMR (CD₃OD, 400 MHz) δ 8.43 (d, 1H), 8.38 (s, 1H), 8.15 (s, 1H), 8.09 (s, 1H), 7.79 (s, 1H), 7.79-7.39 (m, 2H), 7.48 (q, 1H), 6.98 (d, 1H), 6.86 (t, 1H), 4.35-4.10 (m, 2H), 3.83 (s, 3H), 3.83-3.80 (m, 1H), 3.21 (d, 1H), 2.51 (t, 1H), 2.41 (t, 1H), 2.10-1.95 (m, 1H), 1.85-1.75 (m, 1H), 1.70-1.60 (m, 2H), 1.25-1.10 (m, 1H) MS m/z=529.9 (M+H)+

### Example 273: (R)-N-(5-(1-(difluoromethyl)-1H-pyrazol-4-yl)-4-(3-(difluoromethyl) piperidin-1-yl)pyridin-2-yl)-2-(2-fluoro-6-methoxyphenyl)pyrimidin-4-amine

The titled compound (20.2 mg) was prepared in accordance with the same procedures as in Example 7, using Intermediates B3-44 and C1-1. (Yield: 23.9%) 1H NMR (CD₃OD, 400 MHz) δ 8.44 (d, 1H), 8.39 (s, 1H), 8.16 (s, 1H), 8.09 (s, 1H), 7.77 (s, 1H), 7.77-7.39 (m, 2H), 7.46 (q, 1H), 6.98 (d, 1H), 6.86 (t, 1H), 5.80-5.45 (m, 1H), 3.83 (s, 3H), 3.35-3.25 (m, 1H), 3.22 (d, 1H), 2.60-2.45 (m, 2H), 2.20-2.05 (m, 1H), 1.95-1.85 (m, 1H), 1.75-1.55 (m, 2H), 1.40-1.30 (m, 1H) MS m/z=546.9 (M+H)+

### Example 274: (S)-7-(4-(3-aminopiperidin-1-yl)-6-((2-(2-fluoro-6-methoxyphenyl)pyrimidin-4-yl)amino)pyridin-3-yl)-4-methyl-2H-pyrido[3,2-b][1,4]oxazin-3(4H)-one hydrochloride

The titled compound (10.0 mg) was prepared in accordance with the same procedures as in Example 52, using Intermediates A3-49 and C1-1. (Yield: 50.4%) 1H NMR (CD₃OD, 400 MHz) δ 8.74 (d, 1H), 8.23 (s, 1H), 8.14 (s, 1H), 7.66-7.58 (m 3H), 7.20 (s, 1H), 7.11 (d, 1H), 6.98 (t, 1H), 4.82 (s, 2H), 3.93 (s, 3H), 3.85-3.70 (m, 1H), 3.58-3.40 (m, 4H), 3.40-3.25 (m, 1H), 3.05-2.85 (m, 2H), 2.18 (d, 1H), 1.90-1.75 (m, 1H), 1.75-1.50 (m, 2H) MS m/z=557.9 (M+H)+

### Example 275: (S)-7-(4-(3-aminopiperidin-1-yl)-6-((2-(2-fluoro-6-methoxyphenyl)pyrimidin-4-yl)amino)pyridin-3-yl)-2H-pyrido[3,2-b][1,4]oxazin-3(4H)-one hydrochloride

The titled compound (2.4 mg) was prepared in accordance with the same procedures as in Example 52, using Intermediates A3-50 and C1-1. (Yield: 95.8%) 1H NMR (CD₃OD, 400 MHz) δ 8.47 (d, 1H), 8.13 (s, 1H), 8.04 (s, 1H), 7.66 (s, 1H), 7.66-7.61 (m 1H), 7.61-7.58 (m, 1H), 7.46 (q, 1H), 6.99 (d, 1H), 6.87 (t, 1H), 4.73 (s, 2H), 3.84 (s, 3H), 3.25-3.15 (m, 1H), 3.05-2.90 (m, 1H), 2.67 (t, 1H), 2.50 (t, 1H), 2.10-2.00 (m, 2H), 1.75-1.65 (m, 1H), 1.65-1.50 (m, 2H) MS m/z=543.9 (M+H)+

### Example 276: (S)-6-(4-(3-aminopiperidin-1-yl)-6-((2-(2-fluoro-6-methoxyphenyl)pyrimidin-4-yl)amino)pyridin-3-yl)-3,4-dihydro-1,8-naphthyridin-2(1H)-one hydrochloride

The titled compound (9.1 mg) was prepared in accordance with the same procedures as in Example 52, using Intermediates A3-51 and C1-1. (Yield: 65.2%) 1H NMR (CD₃OD, 400 MHz) δ 8.74 (d, 1H), 8.35 (s, 1H), 8.13 (s, 1H), 7.88 (s, 1H), 7.80-7.40 (m 2H), 7.20 (s, 1H), 7.12 (d, 1H), 6.99 (t, 1H), 3.93 (s, 3H), 3.76 (d, 1H), 3.55-3.45 (m, 1H), 3.40-3.20 (m, 1H), 3.10 (t, 2H), 3.00-2.85 (m, 2H), 2.72 (t, 2H), 2.18 (d, 1H), 1.85-1.75 (m, 1H), 1.70-1.50 (m, 2H) MS m/z=542.0 (M+H)+

### Example 277: (S)-N-(4-(3-aminopiperidin-1-yl)-5-(1-(oxetan-3-yl)-1H-pyrazol-4-yl)pyridin-2-yl)-2-(2-fluoro-6-methoxyphenyl)pyrimidin-4-amine hydrochloride

The titled compound (6.5 mg) was prepared in accordance with the same procedures as in Example 52, using Intermediates A3-52 and C1-1. (Yield: 21.2%) 1H NMR (CD₃OD, 400 MHz) δ 8.79 (d, 1H), 8.20 (d, 1H), 8.16 (s, 1H), 7.90 (s, 1H), 7.68 (q, 1H), 7.55 (brs, 1H), 7.25 (s, 1H), 7.15 (d, 1H), 7.02 (t, 1H), 4.74-4.60 (m, 1H), 4.05 (d, 2H), 4.01 (d, 2H), 3.96 (s, 3H), 3.89 (d, 1H), 3.63 (d, 1H), 3.50-3.40 (m, 1H), 3.05-2.90 (m, 2H), 2.20 (d, 1H), 1.95-1.75 (m, 2H), 1.70-1.55 (m, 1H) MS m/z=553.9 (M+C1)-

### Example 278: methyl (S)-2-(4-(4-(3-aminopiperidin-1-yl)-6-((2-(2-fluoro-6-methoxyphenyl)pyrimidin-4-yl)amino)pyridin-3-yl)-1H-pyrazol-1-yl)-2-methylpropanoate hydrochloride

The titled compound (18.9 mg) was prepared in accordance with the same procedures as in Example 52, using Intermediates A3-53 and C1-1. (Yield: 88.2%) 1H NMR (CD₃OD, 400 MHz) δ 8.78 (d, 1H), 8.26 (s, 1H), 8.15 (s, 1H), 7.84 (s, 1H), 7.68 (q, 1H), 7.54 (brs, 1H), 7.23 (s, 1H), 7.15 (d, 1H), 7.02 (t, 1H), 3.96 (s, 3H), 3.90 (d, 1H), 3.78 (s, 3H), 3.64 (d, 1H), 3.45-3.35 (m, 1H), 3.05-2.90 (m, 2H), 2.21 (d, 1H), 1.94 (s, 3H), 1.93 (s, 3H), 1.90-1.85 (m, 1H), 1.85-1.70 (m, 1H), 1.70-1.55 (m, 1H) MS m/z=562.0 (M+H)+

### Example 279: (S)-N-(4-(4-(3-aminopiperidin-1-yl)-6-((2-(2-fluoro-6-methoxyphenyl)pyrimidin-4-yl)amino)pyridin-3-yl)phenyl)acetamide hydrochloride

The titled compound (15.4 mg) was prepared in accordance with the same procedures as in Example 52, using Intermediates A3-54 and C1-1. (Yield: 92.9%) 1H NMR (CD₃OD, 400 MHz) δ 8.73 (d, 1H), 8.04 (s, 1H), 7.80-7.70 (m, 2H), 7.60-7.50 (m, 3H), 7.50-7.20 (m, 1H), 7.10-7.00 (m, 2H), 6.95 (t, 1H), 3.90 (s, 3H), 3.80-3.65 (m, 1H), 3.60-3.45 (m, 1H), 3.30-3.20 (m, 1H), 2.95-2.80 (m, 2H), 2.18 (s, 3H), 2.18-2.10 (m, 1H), 1.85-1.75 (m, 1H), 1.75-1.60 (m, 1H), 1.60-1.45 (m, 1H) MS m/z=529.0 (M+H)+

### Example 280: (S)-3-(4-(3-aminopiperidin-1-yl)-6-((2-(2-fluoro-6-methoxyphenyl)pyrimidin-4-yl)amino)pyridin-3-yl)-N,N-dimethylbenzamide hydrochloride

The titled compound (12.0 mg) was prepared in accordance with the same procedures as in Example 52, using Intermediates A3-55 and C1-1. (Yield: 74.1%) 1H NMR (CD₃OD, 400 MHz) δ 8.77 (d, 1H), 8.12 (s, 1H), 7.70-7.60 (m, 4H), 7.60-7.50 (m, 2H), 7.20 (s, 1H), 7.12 (d, 1H), 6.99 (t, 1H), 3.94 (s, 3H), 3.67 (d, 1H), 3.51 (d, 1H), 3.40-3.35 (m, 1H), 3.16 (s, 3H), 3.07 (s, 3H), 3.00-2.85 (m, 2H), 2.14 (d, 1H), 1.85-1.75 (m, 1H), 1.75-1.65 (m, 1H), 1.65-1.50 (m, 1H) MS m/z=542.9 (M+H)+

### Example 281: (S)-N-(4-(3-aminopiperidin-1-yl)-5-(3-methyl-4-(trifluoromethoxy)phenyl)pyridin-2-yl)-2-(2-fluoro-6-methoxyphenyl)pyrimidin-4-amine hydrochloride

The titled compound (15.6 mg) was prepared in accordance with the same procedures as in Example 52, using Intermediates A3-56 and C1-1. (Yield: 42.1%) 1H NMR (CD₃OD, 400 MHz) δ 8.79 (d, 1H), 8.08 (s, 1H), 7.67 (q, 1H), 7.65-7.55 (m, 2H), 7.54-7.43 (m, 2H), 7.24 (s, 1H), 7.14 (d, 1H), 7.01 (t, 1H), 3.96 (s, 3H), 3.86 (d, 1H), 3.51 (d, 1H), 3.40-3.30 (m, 1H), 3.04 (t, 1H), 2.93 (t, 1H), 2.42 (s, 3H), 2.25-2.10 (m, 1H), 1.80-1.70 (m, 1H), 1.70-1.55 (m, 2H) MS m/z=569.9 (M+H)+

### Example 282: (S)-N-(4-(3-aminopiperidin-1-yl)-5-(4-fluoro-3-methylphenyl)pyridin-2-yl)-2-(2-fluoro-6-methoxyphenyl)pyrimidin-4-amine hydrochloride

The titled compound (45.7 mg) was prepared in accordance with the same procedures as in Example 52, using Intermediates A3-57 and C1-1. (Yield: 78.2%) 1H NMR (CD₃OD, 400 MHz) δ 8.81 (d, 1H), 8.05 (s, 1H), 7.69 (q, 1H), 7.65-7.40 (m, 3H), 7.30-7.20 (m, 2H), 7.15 (d, 1H), 7.02 (t, 1H), 3.97 (s, 3H), 3.88 (d, 1H), 3.56 (d, 1H), 3.40-3.30 (m, 1H), 3.03 (t, 1H), 2.94 (t, 1H), 2.37 (s, 3H), 2.25-2.10 (m, 1H), 1.80-1.70 (m, 1H), 1.70-1.55 (m, 2H) MS m/z=503.0 (M+H)+

### Example 283: (S)-N-(4-(3-aminopiperidin-1-yl)-5-(1-(piperidin-4-yl)-1H-pyrazol-4-yl)pyridin-2-yl)-2-(2-fluoro-6-methoxyphenyl)pyrimidin-4-amine hydrochloride

The titled compound (31.8 mg) was prepared in accordance with the same procedures as in Example 52, using Intermediates A3-58 and C1-1. (Yield: 74.2%) 1H NMR (CD₃OD, 400 MHz) δ 8.75 (d, 1H), 8.24 (s, 1H), 8.17 (s, 1H), 7.87 (s, 1H), 7.63 (q, 1H), 7.60-7.45 (m, 1H), 7.18 (s, 1H), 7.12 (d, 1H), 6.99 (t, 1H), 4.80-4.65 (m, 1H), 3.94 (s, 3H), 3.85 (d, 1H), 3.70-3.55 (m, 2H), 3.55-3.45 (m, 2H), 3.30-3.20 (m, 2H), 3.01 (t, 1H), 2.93 (t, 1H), 2.45-2.30 (m, 4H), 2.25-2.15 (m, 1H), 1.90-1.70 (m, 2H), 1.70-1.55 (m, 1H) MS m/z=545.0 (M+H)+

### Example 284: (S)-N-(4-(3-aminopiperidin-1-yl)-5-(4-isopropoxy-3,5-dimethylphenyl)pyridin-2-yl)-2-(2-fluoro-6-methoxyphenyl)pyrimidin-4-amine hydrochloride

The titled compound (25.5 mg) was prepared in accordance with the same procedures as in Example 52, using Intermediates A3-59 and C1-1. (Yield: 61.7%) 1H NMR (CD₃OD, 400 MHz) δ 8.77 (d, 1H), 7.99 (s, 1H), 7.62 (q, 1H), 7.43 (brs, 1H), 7.21 (s, 2H), 7.15-7.05 (m, 2H), 6.98 (t, 1H), 4.40-4.25 (m, 1H), 3.93 (s, 3H), 3.90 (d, 1H), 3.56 (d, 1H), 3.40-3.25 (m, 1H), 2.99 (t, 1H), 2.87 (t, 1H), 2.34 (s, 6H), 2.20-2.10 (m, 1H), 1.80-1.65 (m, 1H), 1.65-1.50 (m, 2H), 1.33 (s, 3H), 1.32 (s, 3H) MS m/z=557.3 (M+H)+

### Example 285: (S)-N-(4-(3-aminopiperidin-1-yl)-5-(4-methoxy-3,5-dimethylphenyl)pyridin-2-yl)-2-(2-fluoro-6-methoxyphenyl)pyrimidin-4-amine hydrochloride

The titled compound (37.5 mg) was prepared in accordance with the same procedures as in Example 52, using Intermediates A3-60 and C1-1. (Yield: 65.3%) 1H NMR (CD₃OD, 400 MHz) δ 8.79 (d, 1H), 7.99 (s, 1H), 7.66 (q, 1H), 7.55-7.45 (m, 1H), 7.21 (s, 2H), 7.19 (s, 1H), 7.13 (d, 1H), 7.01 (t, 1H), 3.95 (s, 3H), 3.91 (d, 1H), 3.78 (s, 3H), 3.59 (d, 1H), 3.40-3.25 (m, 1H), 3.02 (t, 1H), 2.91 (t, 1H), 2.36 (s, 6H), 2.20-2.10 (m, 1H), 1.80-1.65 (m, 1H), 1.65-1.50 (m, 2H) MS m/z=529.3 (M+H)+

### Example 286: (S)-N-(4-(3-aminopiperidin-1-yl)-5-(4-morpholinophenyl)pyridin-2-yl)-2-(2-fluoro-6-methoxyphenyl)pyrimidin-4-amine hydrochloride

The titled compound (26.4 mg) was prepared in accordance with the same procedures as in Example 52, using Intermediates A3-61 and C1-1. (Yield: 80.8%) 1H NMR (CD₃OD, 400 MHz) δ 8.80 (d, 1H), 8.07 (s, 1H), 7.96 (q, 1H), 7.65-7.60 (m, 2H), 7.60-7.55 (m, 1H), 7.55-7.45 (m, 2H), 7.27 (s, 1H), 7.15 (d, 1H), 7.02 (t, 1H), 4.10-4.00 (m, 4H), 3.97 (s, 3H), 3.87 (d, 1H), 3.60-3.45 (m, 5H), 3.40-3.30 (m, 1H), 3.03 (t, 1H), 2.94 (t, 1H), 2.20-2.10 (m, 1H), 1.80-1.70 (m, 1H), 1.70-1.50 (m, 2H) MS m/z=556.3 (M+H)+

### Example 287: (S)-N-(4-(3-aminopiperidin-1-yl)-5-(3-methyl-4-morpholinophenyl)pyridin-2-yl)-2-(2-fluoro-6-methoxyphenyl)pyrimidin-4-amine hydrochloride

The titled compound (25.4 mg) was prepared in accordance with the same procedures as in Example 52, using Intermediates A3-62 and C1-1. (Yield: 58.0%) 1H NMR (CD₃OD, 400 MHz) δ 8.80 (d, 1H), 8.04 (s, 1H), 7.68 (q, 1H), 7.53 (brs, 1H), 7.50-7.40 (m, 2H), 7.40-7.35 (m, 1H), 7.23 (s, 1H), 7.15 (d, 1H), 7.02 (t, 1H), 4.05-3.90 (m, 7H), 3.88 (d, 1H), 3.56 (d, 1H), 3.40-3.35 (m, 1H), 3.25-3.10 (m, 4H), 3.02 (t, 1H), 2.93 (t, 1H), 2.49 (s, 3H), 2.20-2.10 (m, 1H), 1.80-1.70 (m, 1H), 1.70-1.55 (m, 2H) MS m/z=570.3 (M+H)+

### Example 288: (S)-(4-(4-(3-aminopiperidin-1-yl)-6-((2-(2-fluoro-6-methoxyphenyl)pyrimidin-4-yl)amino)pyridin-3-yl)phenyl)(4-(2-hydroxyethyl)piperazin-1-yl)methan one hydrochloride

The titled compound (16.2 mg) was prepared in accordance with the same procedures as in Example 52, using Intermediates A3-63 and C1-1. (Yield: 74.1%) 1H NMR (CD₃OD, 400 MHz) δ 8.71 (d, 1H), 8.10 (s, 1H), 7.80-7.65 (m, 4H), 7.65-7.35 (m, 2H), 7.16 (brs, 1H), 7.08 (d, 1H), 6.95 (t, 1H), 4.08 (t, 2H), 3.95 (s, 3H), 3.80-3.55 (m, 4H), 3.55-3.40 (m, 2H), 3.39 (t, 2H), 3.39-3.20 (m, 5H), 2.95-2.80 (m, 2H), 2.20-2.10 (m, 1H), 1.85-1.70 (m, 1H), 1.70-1.50 (m, 2H) MS m/z=627.4 (M+H)+

### Example 289: 4-(4-((S)-3-aminopiperidin-1-yl)-6-((2-(2-fluoro-6-methoxyphenyl)pyrimidin-4-yl)amino)pyridin-3-yl)-N-((1r,4S)-4-hydroxycyclohexyl)benzamide hydrochloride

The titled compound (24.3 mg) was prepared in accordance with the same procedures as in Example 52, using Intermediates A3-64 and C1-1. (Yield: 62.5%) 1H NMR (CD₃OD, 400 MHz) δ 8.79 (d, 1H), 8.11 (s, 1H), 7.99 (d, 2H), 7.70-7.60 (m, 3H), 7.56 (brs, 1H), 7.24 (s, 1H), 7.13 (d, 1H), 7.00 (t, 1H), 3.95 (s, 3H), 3.95-3.85 (m, 1H), 3.85-3.75 (m, 1H), 3.65-3.55 (m, 1H), 3.50 (d, 1H), 3.05-2.90 (m, 2H), 2.20-2.10 (m, 1H), 2.10-1.95 (m, 4H), 1.80-1.70 (m, 1H), 1.80-1.55 (m, 3H), 1.55-1.35 (m, 4H) MS m/z=612.3 (M+H)+

### Example 290: (S)-4-(4-(3-aminopiperidin-1-yl)-6-((2-(2-fluoro-6-methoxyphenyl)pyrimidin-4-yl)amino)pyridin-3-yl)-2-fluoro-N,N-dimethylbenzamide hydrochloride

The titled compound (21.6 mg) was prepared in accordance with the same procedures as in Example 52, using Intermediates A3-65 and C1-1. (Yield: 55.1%) 1H NMR (CD₃OD, 400 MHz) δ 8.76 (d, 1H), 8.17 (s, 1H), 7.66 (q, 1H), 7.65-7.55 (m, 2H), 7.55-7.45 (m, 2H), 7.25 (s, 1H), 7.13 (d, 1H), 7.00 (t, 1H), 3.95 (s, 3H), 3.69 (d, 1H), 3.52 (d, 1H), 3.30-3.25 (m, 1H), 3.18 (s, 3H), 3.03 (s, 3H), 3.00-2.85 (m, 2H), 2.17 (d, 1H), 1.85-1.75 (m, 1H), 1.75-1.65 (m, 1H), 1.65-1.50 (m, 1H) MS m/z=560.9 (M+H)+

### Example 291: (S)-1-(3-(4-(3-aminopiperidin-1-yl)-6-((2-(2-fluoro-6-methoxyphenyl)pyrimidin-4-yl)amino)pyridin-3-yl)phenyl)ethan-1-one hydrochloride

The titled compound (21.6 mg) was prepared in accordance with the same procedures as in Example 52, using Intermediates A3-66 and C1-1. (Yield: 36.3%) 1H NMR (CD₃OD, 400 MHz) δ 8.78 (d, 1H), 8.19 (s, 1H), 8.15 (s, 1H), 7.80-7.60 (m, 3H), 7.60-7.50 (m, 2H), 7.25 (s, 1H), 7.13 (d, 1H), 7.01 (t, 1H), 3.95 (s, 3H), 3.80 (d, 1H), 3.49 (d, 1H), 3.10-2.80 (m, 3H), 2.69 (s, 3H), 2.20-2.10 (m, 1H), 1.75-1.65 (m, 1H), 1.65-1.50 (m, 2H) MS m/z=514.0 (M+H)+

### Example 292: (S)-N-(4-(3-aminopiperidin-1-yl)-5-(3-chloro-4-fluorophenyl)pyridin-2-yl)-2-(2-fluoro-6-methoxyphenyl)pyrimidin-4-amine hydrochloride

The titled compound (9.5 mg) was prepared in accordance with the same procedures as in Example 52, using Intermediates A3-67 and C1-1. (Yield: 47.5%) 1H NMR (CD₃OD, 400 MHz) δ 8.78 (d, 1H), 8.11 (s, 1H), 7.78 (d, 1H), 7.67 (q, 1H), 7.64-7.50 (m, 2H), 7.45 (t, 1H), 7.24 (s, 1H), 7.14 (d, 1H), 7.01 (t, 1H), 3.96 (s, 3H), 3.80 (d, 1H), 3.51 (d, 1H), 3.40-3.30 (m, 1H), 3.05-2.90 (m, 2H), 2.25-2.10 (m, 1H), 1.85-1.70 (m, 1H), 1.70-1.55 (m, 2H) MS m/z=523.9 (M+H)+

### Example 293: (S)-N-(4-(3-aminopiperidin-1-yl)-5-(4-fluoro-3-(trifluoromethyl)phenyl)pyridin-2-yl)-2-(2-fluoro-6-methoxyphenyl)pyrimidin-4-amine hydrochloride

The titled compound (18.7 mg) was prepared in accordance with the same procedures as in Example 52, using Intermediates A3-68 and C1-1. (Yield: 55.6%) 1H NMR (CD₃OD, 400 MHz) δ 8.81 (d, 1H), 8.16 (s, 1H), 8.00-7.90 (m, 2H), 7.70 (q, 1H), 7.63 (brs, 1H), 7.57 (t, 1H), 7.32 (s, 1H), 7.16 (d, 1H), 7.03 (t, 1H), 3.98 (s, 3H), 3.83 (d, 1H), 3.48 (d, 1H), 3.40-3.25 (m, 1H), 3.04 (t, 1H), 2.95 (t, 1H), 2.25-2.10 (m, 1H), 1.80-1.70 (m, 11H), 1.70-1.55 (m, 2H) MS m/z=557.2 (M+H)+

### Example 294: (S)-N-(4-(3-aminopiperidin-1-yl)-5-(3,4-difluorophenyl)pyridin-2-yl)-2-(2-fluoro-6-methoxyphenyl)pyrimidin-4-amine hydrochloride

The titled compound (18.9 mg) was prepared in accordance with the same procedures as in Example 52, using Intermediates A3-69 and C1-1. (Yield: 56.4%) 1H NMR (CD₃OD, 400 MHz) δ 8.83 (d, 1H), 8.11 (s, 1H), 7.71 (q, 1H), 7.65-7.55 (m, 2H), 7.49 (q, 1H), 7.45-7.40 (m, 1H), 7.32 (s, 1H), 7.17 (d, 1H), 7.04 (t, 1H), 3.98 (s, 3H), 3.86 (d, 1H), 3.55 (d, 1H), 3.45-3.35 (m, 1H), 3.06 (t, 1H), 2.99 (t, 1H), 2.25-2.10 (m, 1H), 1.85-1.75 (m, 1H), 1.75-1.55 (m, 2H) MS m/z=507.9 (M+H)+

### Example 295: (3R,4R)-4-amino-1-(5-(1-(difluoromethyl)-1H-pyrazol-4-yl)-2-((2-(2-fluoro-6-methoxyphenyl)pyrimidin-4-yl)amino)pyridin-4-yl)piperidin-3-ol

The titled compound (13.4 mg) was prepared in accordance with the same procedures as in Example 52, using Intermediates B1-9 and C1-1. (Yield: 13.0%) 1H NMR (CD₃OD, 400 MHz) δ 8.61 (d, 1H), 8.13 (s, 1H), 8.10 (s, 1H), 7.97 (s, 1H), 7.54 (s, 1H), 7.50-7.35 (m, 3H), 7.40-7.10 (m, 1H), 6.84 (t, 1H), 3.85 (s, 3H), 3.46 (d, 1H), 3.40-3.30 (m, 1H), 3.27 (d, 1H), 2.62 (t, 1H), 2.55-2.45 (m, 2H), 1.85-1.75 (m, 1H), 1.50-1.35 (m, 1H) MS m/z=527.2 (M+H)+

### Example 296: (S)-N-(4-(3-aminopiperidin-1-yl)-5-(3-(dimethylamino)-4-fluorophenyl)pyridin-2-yl)-2-(2-fluoro-6-methoxyphenyl)pyrimidin-4-amine hydrochloride

The titled compound (31.6 mg) was prepared in accordance with the same procedures as in Example 52, using Intermediates A3-70 and C1-1. (Yield: 76.3%) 1H NMR (CD₃OD, 400 MHz) δ 8.78 (d, 1H), 8.17 (s, 1H), 7.95-7.85 (m, 1H), 7.70-7.55 (m, 3H), 7.53 (q, 1H), 7.28 (s, 1H), 7.14 (d, 1H), 7.01 (t, 1H), 3.96 (s, 3H), 3.82 (d, 1H), 3.49 (d, 1H), 3.45-3.35 (m, 1H), 3.33 (s, 6H), 3.10-2.90 (m, 2H), 2.25-2.10 (m, 1H), 1.80-1.70 (m, 1H), 1.70-1.55 (m, 2H) MS m/z=532.3 (M+H)+

### Example 297: N-(4-((3R,4S)-4-amino-3-methylpiperidin-1-yl)-5-(1-(difluoromethyl)-1H-pyrazol-4-yl)pyridin-2-yl)-2-(2-fluoro-6-methoxyphenyl)pyrimidin-4-amine hydrochloride

The titled compound (33.1 mg) was prepared in accordance with the same procedures as in Example 52, using Intermediates B1-10 and C1-1. (Yield: 58.5%) 1H NMR (CD₃OD, 400 MHz) δ 8.79 (d, 1H), 8.45 (s, 1H), 8.15 (s, 1H), 8.01 (s, 1H), 7.77-7.47 (m, 1H), 7.69 (q, 1H), 7.56 (brs, 1H), 7.31 (s, 1H), 7.15 (d, 1H), 7.02 (t, 1H), 3.97 (s, 3H), 3.65-3.50 (m, 2H), 3.50-3.40 (m, 1H), 3.35-3.30 (m, 1H), 3.20-3.15 (m, 1H), 2.40-2.30 (m, 1H), 1.95-1.85 (m, 2H), 1.04 (d, 3H) MS m/z=525.2 (M+H)+

### Example 298: N-(4-((S)-3-aminopiperidin-1-yl)-5-(1-((S)-tetrahydrofuran-3-yl)-1H-pyrazol-4-yl)pyridin-2-yl)-2-(2-fluoro-6-methoxyphenyl)pyrimidin-4-amine hydrochloride

The titled compound (33.3 mg) was prepared in accordance with the same procedures as in Example 52, using Intermediates A3-71 and C1-1. (Yield: 93.1%) 1H NMR (CD₃OD, 400 MHz) δ 8.79 (d, 1H), 8.16 (s, 1H), 8.14 (s, 1H), 7.85 (s, 1H), 7.67 (q, 1H), 7.54 (brs, 1H), 7.26 (s, 1H), 7.15 (d, 1H), 7.02 (t, 1H), 5.18-5.14 (m, 1H), 4.25-4.15 (m, 1H), 4.15-4.00 (m, 2H), 3.98 (s, 3H), 3.98-3.89 (m, 2H), 3.62 (d, 1H), 3.50-3.40 (m, 1H), 3.04 (t, 1H), 2.97 (t, 1H), 2.60-2.50 (m, 1H), 2.50-2.35 (m, 1H), 2.25-2.15 (m, 1H), 1.90-1.80 (m, 1H), 1.80-1.70 (m, 1H), 1.70-1.60 (m, 1H) MS m/z=532.0 (M+H)+

### Example 299: N-(4-((S)-3-aminopiperidin-1-yl)-5-(1-((R)-tetrahydrofuran-3-yl)-1H-pyrazol-4-yl)pyridin-2-yl)-2-(2-fluoro-6-methoxyphenyl)pyrimidin-4-amine hydrochloride

The titled compound (18.2 mg) was prepared in accordance with the same procedures as in Example 52, using Intermediates A3-72 and C1-1. (Yield: 61.2%) 1H NMR (CD₃OD, 400 MHz) δ 8.78 (d, 1H), 8.16 (s, 1H), 8.14 (s, 1H), 7.85 (s, 1H), 7.67 (q, 1H), 7.53 (brs, 1H), 7.25 (s, 1H), 7.14 (d, 1H), 7.01 (t, 1H), 5.18-5.14 (m, 1H), 4.25-4.15 (m, 1H), 4.15-4.00 (m, 2H), 3.98 (s, 3H), 3.98-3.85 (m, 2H), 3.60 (d, 1H), 3.50-3.40 (m, 1H), 3.05-2.90 (m, 2H), 2.65-2.50 (m, 1H), 2.50-2.35 (m, 1H), 2.25-2.15 (m, 1H), 1.90-1.80 (m, 1H), 1.80-1.70 (m, 1H), 1.70-1.60 (m, 1H) MS m/z=531.2 (M+H)+

### Example 300: 6-((4-((S)-3-aminopiperidin-1-yl)-5-(1-(tetrahydro-2H-pyran-4-yl)-1H-pyrazol-4-yl)pyridin-2-yl)amino)-2-(2-fluoro-6-methoxyphenyl)nicotinonitrile hydrochloride

### Step 1: tert-butyl N-[(3S)-1-[2-[[5-cyano-6-(2-fluoro-6-methoxy-phenyl)-2-pyridyl]amino]-5-(1-tetrahydropyran-4-ylpyrazol-4-yl)-4-pyridyl]-3-piperidyl]carbamate

The titled compound (42.7 mg) was prepared in accordance with the same procedures as in Step 1 of Example 52, using Intermediates A3-1 and C8-1. (Yield: 59.0%) 1H NMR (CDCl₃, 400 MHz) δ 8.12 (s, 1H), 7.90-7.85 (m, 1H), 7.85-7.75 (m, 2H), 7.77 (s, 1H), 7.50-7.35 (m, 2H), 6.95-6.80 (m, 2H), 4.55-4.45 (m, 1H), 4.45-4.35 (m, 1H), 4.18-4.11 (m, 2H), 3.88 (s, 3H), 3.80-3.68 (m, 1H), 3.63-3.56 (m, 2H), 3.45-3.28 (m, 1H), 3.10-2.95 (m, 1H), 2.60-2.50 (m, 1H), 2.50-2.40 (m, 1H), 2.19-1.94 (m, 4H), 1.98-1.88 (m, 1H), 1.70-1.60 (m, 1H), 1.60-1.53 (m, 1H), 1.48 (s, 9H)

### Step 2: 6-((4-((S)-3-aminopiperidin-1-yl)-5-(1-(tetrahydro-2H-pyran-4-yl)-1H-pyrazol-4-yl)pyridin-2-yl)amino)-2-(2-fluoro-6-methoxyphenyl)nicotinonitrile hydrochloride

tert-Butyl N-[(3S)-1-[2-[[5-cyano-6-(2-fluoro-6-methoxy-phenyl)-2-pyridyl]amino]-5-(1-tetrahydropyran-4-ylpyrazol-4-yl)-4-pyridyl]-3-piperidyl]carbamate (42.7 mg, 0.06 mmol) was dissolved in dichloromethane (3.0 ml) and then 4.0 M hydrogen chloride solution dissolved in 1,4-dioxane (0.48 mL, 1.92 mmol) was added dropwise thereto. Then, the mixed solution was stirred at room temperature for 17 hours. Water was added to the reaction mixture and then the aqueous layer was extracted with ethyl acetate. The extracted organic layer was dried over anhydrous magnesium sulfate and then filtered. The filtrate was concentrated and then purified by silica gel column chromatography to prepare the titled compound (32.0 mg). (Yield: 78.1%) 1H NMR (CD₃OD, 400 MHz) δ 8.47 (s, 1H), 8.28 (d, 1H), 8.11 (s, 1H), 8.03 (s, 1H), 7.81 (s, 1H), 7.59 (q, 1H), 7.35 (d, 1H), 7.10 (s, 1H), 7.06 (d, 1H), 6.97 (t, 1H), 4.55-4.50 (m, 1H), 4.15-4.05 (m, 2H), 3.90 (s, 3H), 3.84 (d, 1H), 3.65-3.55 (m, 2H), 3.55-3.45 (m, 1H), 3.45-3.35 (m, 1H), 2.98 (t, 1H), 2.89 (t, 1H), 2.25-2.15 (m, 1H), 2.15-2.05 (m, 4H), 1.90-1.80 (m, 1H), 1.80-1.70 (m, 1H), 1.70-1.55 (m, 1H) MS m/z=569.0 (M+H)+

### Example 301: 6-((4-((S)-3-aminopiperidin-1-yl)-5-(1-(difluoromethyl)-1H-pyrazol-4-yl)pyridin-2-yl)amino)-2-(2-fluoro-6-methoxyphenyl)nicotinonitrile hydrochloride

### Step 1: tert-butyl N-[(3S)-1-[2-[[5-cyano-6-(2-fluoro-6-methoxy-phenyl)-2-pyridyl]amino]-5-[1-(difluoromethyl)pyrazol-4-yl]-4-pyridyl]-3-piperidyl]carbamate

The titled compound (55.1 mg) was prepared in accordance with the same procedures as in Step 1 of Example 52, using Intermediates A3-2 and C8-1. (Yield: 74.4%) 1H NMR (CDCl₃, 400 MHz) δ 8.11 (s, 1H), 7.95 (s, 1H), 7.90 (d, 1H), 7.77 (s, 1H), 7.53 (s, 1H), 7.47-7.11 (m, 3H), 6.91-6.81 (m, 2H), 4.50-4.40 (m, 1H), 3.89 (s, 3H), 3.80-3.68 (m, 1H), 3.45-3.24 (m, 1H), 3.08-2.96 (m, 1H), 2.58-2.40 (m, 2H), 1.98-1.88 (m, 1H), 1.70-1.60 (m, 1H), 1.60-1.50 (m, 1H), 1.48 (s, 9H)

### Step 2: 6-((4-((S)-3-aminopiperidin-1-yl)-5-(1-(difluoromethyl)-1H-pyrazol-4-yl)pyridin-2-yl)amino)-2-(2-fluoro-6-methoxyphenyl)nicotinonitrile hydrochloride

tert-Butyl N-[(3S)-1-[2-[[5-cyano-6-(2-fluoro-6-methoxy-phenyl)-2-pyridyl]amino]-5-[1-(difluoromethyl)pyrazol-4-yl]-4-pyridyl]-3-piperidyl]carbamate (55.1 mg, 0.09 mmol) was dissolved in dichloromethane (3.0 ml) and then 4.0 M hydrogen chloride solution dissolved in 1,4-dioxane (0.65 mL, 2.60 mmol) was added dropwise thereto. Then, the mixed solution was stirred at room temperature for 17 hours. Water was added to the reaction mixture and then the aqueous layer was extracted with ethyl acetate. The extracted organic layer was dried over anhydrous magnesium sulfate and then filtered. The filtrate was concentrated and then purified by silica gel column chromatography to prepare the titled compound (32.0 mg). (Yield: 60.9%) 1H NMR (CD₃OD, 400 MHz) δ 8.48 (s, 1H), 8.29 (d, 1H), 8.11 (s, 1H), 8.05 (s, 1H), 7.75-7.45 (m, 1H), 7.59 (q, 1H), 7.38 (d, 1H), 7.10 (s, 1H), 7.09 (d, 1H), 6.97 (t, 1H), 3.91 (s, 3H), 3.81 (d, 1H), 3.52 (d, 1H), 3.50-3.40 (m, 1H), 3.05-2.90 (m, 2H), 2.25-2.15 (m, 1H), 1.90-1.80 (m, 1H), 1.80-1.70 (m, 1H), 1.70-1.55 (m, 1H) MS m/z=534.9 (M+H)+

### Example 302: N-(4-((3S,4S)-4-amino-3-fluoropiperidin-1-yl)-5-(1-(difluoromethyl)-1H-pyrazol-4-yl)pyridin-2-yl)-2-(2-fluoro-6-methoxyphenyl)pyrimidin-4-amine hydrochloride

The titled compound (16.3 mg, Yield: 91.6%) was prepared in accordance with the same procedures as in Example 52, using Intermediates B3-45 and C1-1. 1H NMR (CD₃OD, 400 MHz) δ 8.78 (d, 1H), 8.42 (s, 1H), 7.99 (s, 1H), 7.94 (s, 1H), 7.66-7.59 (m, 1H), 7.62 (t, 1H), 7.34 (d, 1H), 7.11 (d, 1H), 7.01-6.95 (m, 2H), 5.00-4.95 (m, 1H), 4.40-4.34 (m, 1H), 3.94 (s, 3H), 3.78-3.72 (m, 1H), 3.50-3.46 (m, 1H), 3.46-3.35 (m, 2H), 2.39-2.35 (m, 1H), 2.06-1.96 (m, 1H) MS m/z=529.9 (M+H)+

### Example 303: N-(4-((3S,5R)-3-amino-5-fluoropiperidin-1-yl)-5-(1-(difluoromethyl)-1H-pyrazol-4-yl)pyridin-2-yl)-2-(2-fluoro-6-methoxyphenyl)pyrimidin-4-amine hydrochloride

The titled compound (12.8 mg, Yield: 78.5%) was prepared in accordance with the same procedures as in Example 52, using Intermediates B3-48 and C1-1. 1H NMR (CDCl₃, 400 MHz) δ 8.64-8.56 (m, 1H), 8.18 (s, 1H), 8.12 (s, 2H), 7.95 (s, 1H), 7.72 (s, 1H), 7.43-7.40 (m, 0.2H), 7.40-7.33 (m, 1H), 7.28-7.22 (m, 1.6H), 7.13-7.10 (m, 0.2H), 6.89-6.79 (m, 2H), 4.75-4.64 (m, 0.5H), 4.64-4.51 (m, 0.5H), 3.88 (s, 3H), 3.46-3.31 (m, 1H), 3.25-3.12 (m, 1H), 3.02-2.91 (m, 1H), 2.81-2.66 (m, 1H), 2.48-2.27 (m, 2H), 1.56-1.40 (m, 2H) MS m/z=529.2 (M+H)+

### Example 304: N-(4-((3S,5S)-3-amino-5-fluoropiperidin-1-yl)-5-(1-(difluoromethyl)-1H-pyrazol-4-yl)pyridin-2-yl)-2-(2-fluoro-6-methoxyphenyl)pyrimidin-4-amine hydrochloride

The titled compound (7.9 mg, Yield: 82.3%) was prepared in accordance with the same procedures as in Example 52, using Intermediates B3-49 and C1-1. 1H NMR (CDCl₃, 400 MHz) δ 8.64-8.55 (m, 1H), 8.26 (s, 1H), 8.20 (s, 1H), 8.08 (s, 1H), 8.00 (s, 1H), 7.58 (s, 1H), 7.44-7.39 (m, 0.2H), 7.39-7.32 (m, 2H), 7.28-7.23 (m, 0.6H), 7.13-7.08 (m, 0.2H), 6.88-6.76 (m, 2H), 4.94-4.73 (m, 1H), 3.88 (s, 3H), 3.53-3.40 (m, 1H), 3.35-3.22 (m, 2H), 2.81-2.63 (m, 1H), 2.39-2.27 (m, 2H), 1.50-1.35 (m, 1H) MS m/z=529.2 (M+H)+

### Example 305: N-(4-((3S,5R)-3-amino-5-(trifluoromethyl)piperidin-1-yl)-5-(1-(difluoromethyl)-1H-pyrazol-4-yl)pyridin-2-yl)-2-(2-fluoro-6-methoxyphenyl)pyrimidin-4-amine hydrochloride

The titled compound (4.7 mg, Yield: 52.3%) was prepared in accordance with the same procedures as in Example 52, using Intermediates B3-50 and C1-1. 1H NMR (CDCl₃, 400 MHz) δ 8.65-8.58 (m, 1H), 8.19 (s, 1H), 8.10 (s, 1H), 7.96 (s, 1H), 7.77 (s, 1H), 7.69 (s, 1H), 7.45-7.41 (m, 0.2H), 7.41-7.33 (m, 1H), 7.27-7.25 (m, 0.6H), 7.25-7.21 (m, 1H), 7.13-7.07 (m, 0.2H), 6.89-6.79 (m, 2H), 3.88 (s, 3H), 3.49-3.40 (m, 1H), 3.38-3.29 (m, 1H), 2.99-2.87 (m, 1H), 2.54-2.45 (m, 1H), 2.45-2.33 (m, 1H), 2.28-2.17 (m, 2H), 1.40-1.31 (m, 1H), 1.24-1.11 (m, 2H) MS m/z=579.2 (M+H)+

### Example 306: (3S,5S)-5-amino-1-(5-(1-(difluoromethyl)-1H-pyrazol-4-yl)-2-((2-(2-fluoro-6-methoxyphenyl)pyrimidin-4-yl)amino)pyridin-4-yl)piperidin-3-ol hydrochloride

The titled compound (12.2 mg, Yield: 63.2%) was prepared in accordance with the same procedures as in Example 52, using Intermediates B3-51 and C1-1. 1H NMR (CDCl₃, 400 MHz) δ 8.63-8.56 (m, 1H), 8.21 (s, 1H), 8.15 (s, 1H), 8.02 (s, 1H), 7.97 (s, 1H), 7.57 (s, 1H), 7.43-7.32 (m, 2.2H), 7.27-7.23 (m, 0.6H), 7.13-7.09 (m, 0.2H), 6.88-6.79 (m, 2H), 3.93-3.86 (m, 1H), 3.86 (s, 3H), 3.22-3.12 (m, 1H), 3.11-3.01 (m, 1H), 3.01-2.89 (m, 2H), 2.81-2.72 (m, 1H), 1.99-1.90 (m, 1H), 1.60-1.50 (m, 1H) MS m/z=527.9 (M+H)+

### Example 307: N-(4-((3R,4S)-4-amino-3-fluoropiperidin-1-yl)-5-(1-(difluoromethyl)-1H-pyrazol-4-yl)pyridin-2-yl)-2-(2-fluoro-6-methoxyphenyl)pyrimidin-4-amine hydrochloride

The titled compound (12.9 mg, Yield: 37.6%) was prepared in accordance with the same procedures as in Example 52, using Intermediates B3-46 and C1-1. 1H NMR (CD₃OD, 400 MHz) δ 8.74 (d, 1H), 8.46 (s, 1H), 8.23 (s, 1H), 8.05 (s, 1H), 7.68-7.55 (m, 2H), 7.59 (t, 1H), 7.28 (s, 1H), 7.13 (d, 1H), 7.00 (t, 1H), 5.11-4.98 (m, 1H), 4.03-3.95 (m, 1H), 3.94 (s, 3H), 3.73-3.62 (m, 2H), 3.40-3.26 (m, 1H), 3.12 (t, 1H), 2.13-2.03 (m, 1H), 1.97-1.94 (m, 1H) MS m/z=529.2 (M+H)+

### Example 308: N-(4-((3S,4R)-4-amino-3-fluoropiperidin-1-yl)-5-(1-(difluoromethyl)-1H-pyrazol-4-yl)pyridin-2-yl)-2-(2-fluoro-6-methoxyphenyl)pyrimidin-4-amine hydrochloride

The titled compound (20.2 mg, Yield: 54.8%) was prepared in accordance with the same procedures as in Example 52, using Intermediates B3-47 and C1-1. 1H NMR (CD₃OD, 400 MHz) δ 8.76 (d, 1H), 8.47 (s, 1H), 8.23 (s, 1H), 8.05 (s, 1H), 7.69-7.50 (m, 2H), 7.60 (t, 1H), 7.31 (s, 1H), 7.14 (d, 1H), 7.01 (t, 1H), 5.12-4.99 (m, 1H), 4.06-4.00 (m, 1H), 3.95 (s, 3H), 3.77-3.63 (m, 2H), 3.44-3.39 (m, 1H), 3.15 (t, 1H), 2.12-2.03 (m, 1H), 1.98-1.95 (m, 1H) MS m/z=529.2 (M+H)+

### Example 309: 6-((4-((S)-3-aminopiperidin-1-yl)-5-(1-(tetrahydro-2H-pyran-4-yl)-1H-pyrazol-4-yl)pyridin-2-yl)amino)-2-(2,4-difluoro-6-methoxyphenyl)nicotinonitrile hydrochloride

The titled compound (31.3 mg, Yield: 84.0%) was prepared in accordance with the same procedures as in Example 52, using Intermediates A3-1 and C8-2. 1H NMR (CD₃OD, 400 MHz) δ 8.29 (d, 1H), 8.12 (s, 1H), 8.05 (s, 1H), 7.81 (s, 1H), 7.35 (d, 1H), 7.02 (s, 1H), 6.96 (d, 1H), 6.83 (t, 1H), 4.56-4.50 (m, 1H), 4.12-4.08 (m, 2H), 3.90 (s, 3H), 3.87-3.83 (m, 1H), 3.65-3.54 (m, 3H), 3.47-3.41 (m, 1H), 2.99 (t, 1H), 2.90 (t, 1H), 2.26-2.18 (m, 1H), 2.18-2.00 (m, 4H), 1.85-1.81 (m, 1H), 1.81-1.72 (m, 1H), 1.72-1.60 (m, 1H) MS m/z=587.2 (M+H)+

### Example 310: 6-((4-((S)-3-aminopiperidin-1-yl)-5-(1-(difluoromethyl)-1H-pyrazol-4-yl)pyridin-2-yl)amino)-2-(2,4-difluoro-6-methoxyphenyl)nicotinonitrile hydrochloride

The titled compound (31.6 mg, Yield: 77.3%) was prepared in accordance with the same procedures as in Example 52, using Intermediates A3-2 and C8-2. 1H NMR (CD₃OD, 400 MHz) δ 8.51 (s, 1H), 8.28 (d, 1H), 8.12 (s, 1H), 8.07 (s, 1H), 7.62 (t, 1H), 7.40 (d, 1H), 7.11 (s, 1H), 6.95 (d, 1H), 6.83 (t, 1H), 3.90 (s, 3H), 3.87-3.78 (m, 1H), 3.55-3.45 (m, 1H), 3.45-3.40 (m, 1H), 3.00 (t, 1H), 2.92 (t, 1H), 2.23-2.19 (m, 1H), 1.86-1.80 (m, 1H), 1.80-1.70 (m, 1H), 1.70-1.59 (m, 1H) MS m/z=553.2 (M+H)+

### Example 311: (S)-N-(4-(3-aminopiperidin-1-yl)-5-((4-methyltetrahydro-2H-pyran-4-yl)ethynyl)pyridin-2-yl)-2-(2,4-difluoro-6-methoxyphenyl)pyrimidin-4-amine

The titled compound (3.1 mg, Yield: 29.9%) was prepared in accordance with the same procedures as in Example 52, using Intermediates A7-6 and C1-7. 1H NMR (CDCl₃, 400 MHz) δ 8.64-8.51 (m, 1H), 8.26 (s, 1H), 7.65 (s, 1H), 7.47 (s, 1H), 7.19-7.10 (m, 1H), 6.63-6.51 (m, 2H), 3.96-3.83 (m, 4H), 3.83-3.72 (m, 4H), 3.05-2.92 (m, 1H), 2.92-2.80 (m, 1H), 2.65-2.53 (m, 1H), 2.06-1.93 (m, 1H), 1.85-1.73 (m, 3H), 1.73-1.61 (m, 3H), 1.43 (s, 3H) MS m/z=535.3 (M+H)+

### Example 312: (S)-4-((4-(3-aminopiperidin-1-yl)-6-((2-(2,4-difluoro-6-methoxyphenyl)pyrimidin-4-yl)amino)pyridin-3-yl)ethynyl)tetrahydro-2H-pyran-4-ol

The titled compound (4.4 mg, Yield: 54.8%) was prepared in accordance with the same procedures as in Example 52, using Intermediates A7-7 and C1-7. 1H NMR (CDCl₃, 400 MHz) δ 8.64-8.54 (m, 1H), 8.24 (s, 1H), 7.69 (s, 1H), 7.46 (s, 1H), 7.26 (s, 1H), 6.62-6.51 (m, 2H), 5.50 (s, 1H), 4.99 (s, 1H), 4.39-4.29 (m, 1H), 4.03-3.89 (m, 2H), 3.84 (s, 3H), 3.80-3.68 (m, 2H), 3.32-3.23 (m, 2H), 3.00-2.85 (m, 2H), 1.96-1.87 (m, 2H), 1.81-1.75 (m, 1H), 1.75-1.67 (m, 1H), 1.67-1.59 (m, 1H) MS m/z=537.2 (M+H)+

### Example 313: (S)-4-(4-(3-aminopiperidin-1-yl)-6-((2-(2,4-difluoro-6-methoxyphenyl)pyrimidin-4-yl)amino)pyridin-3-yl)-2-methylbut-3-yn-2-ol

The titled compound (3.5 mg, Yield: 34.8%) was prepared in accordance with the same procedures as in Example 52, using Intermediates A7-8 and C1-7. 1H NMR (CDCl₃, 400 MHz) δ 8.63-8.54 (m, 1H), 8.18 (s, 1H), 7.53 (s, 1H), 7.47 (s, 1H), 7.20 (s, 1H), 6.63-6.49 (m, 2H), 4.31-4.20 (m, 1H), 3.86 (s, 3H), 3.31-3.17 (m, 2H), 3.06-2.86 (m, 2H), 2.03-1.92 (m, 1H), 1.87-1.76 (m, 2H), 1.75-1.65 (m, 2H), 1.62 (d, 6H) MS m/z=495.2 (M+H)+

### Example 314: (S)-4-(4-(3-aminopiperidin-1-yl)-6-((2-(2,4-difluoro-6-methoxyphenyl)pyrimidin-4-yl)amino)pyridin-3-yl)but-3-yn-1-ol

The titled compound (8.0 mg, Yield: 42.9%) was prepared in accordance with the same procedures as in Example 52, using Intermediates A7-9 and C1-7. 1H NMR (CDCl₃, 400 MHz) δ 8.62-8.53 (m, 1H), 8.26 (s, 1H), 7.81 (s, 1H), 7.48 (s, 1H), 7.23 (s, 1H), 6.61-6.46 (m, 2H), 3.90-3.83 (m, 1H), 3.83-3.76 (m, 5H), 3.53-3.39 (m, 1H), 3.25-3.16 (m, 1H), 3.16-3.08 (m, 1H), 3.08-2.97 (m, 1H), 2.81-2.66 (m, 2H), 1.99-1.79 (m, 2H), 1.76-1.63 (m, 1H), 1.56-1.46 (m, 1H) MS m/z=481.2 (M+H)+

### Example 315: 4-((S)-3-aminopiperidin-1-yl)-5-(1-(difluoromethyl)-1H-pyrazol-4-yl)-N-(6-(2-fluoro-6-methoxyphenyl)-5-(methylsulfonyl)pyridin-2-yl)pyridin-2-amine hydrochloride

The titled compound (16.8 mg, Yield: 59.7%) was prepared in accordance with the same procedures as in Example 52, using Intermediates A3-2 and C1-5. 1H NMR (CD₃OD, 400 MHz) δ 8.47-8.45 (m, 2H), 8.12 (s, 1H), 8.06 (s, 1H), 7.59 (t, 1H), 7.59-7.49 (m, 2H), 7.31 (s, 1H), 7.00 (d, 1H), 6.89 (t, 1H), 3.81 (s, 3H), 3.70-3.60 (m, 1H), 3.40-3.33 (m, 2H), 2.99 (s, 3H), 2.90-3.75 (m, 2H), 2.22-2.15 (m, 1H), 1.85-1.65 (m, 2H), 1.65-1.50 (m, 1H) MS m/z=588.2 (M+H)+

### Example 316: 2-(2-fluoro-6-methoxyphenyl)-6-((4-((S)-3-hydroxypiperidin-1-yl)-5-(1-(2,2,2-trifluoroethyl)-1H-pyrazol-4-yl)pyridin-2-yl)amino)nicotinonitrile

The titled compound (10.4 mg, Yield: 15.2%) was prepared in accordance with the same procedures as in Example 52, using Intermediates A2-6 and C8-1. 1H NMR (CD₃OD, 400 MHz) δ 8.15 (s, 1H), 8.10 (s, 1H), 8.05-7.90 (m, 2H), 7.75 (s, 1H), 7.60-7.45 (m, 2H), 7.02 (d, 1H), 6.92 (t, 1H), 5.01 (q, 2H), 3.88 (s, 3H), 3.75-3.65 (m, 1H), 3.30-3.20 (m, 1H), 3.05-2.95 (m, 1H), 2.65-2.55 (m, 1H), 2.44 (t, 1H), 1.97-1.85 (m, 1H), 1.75-1.62 (m, 1H), 1.62-1.50 (m, 1H), 1.40-1.30 (m, 1H) MS m/z=568.2 (M+H)+

### Example 317: 2-(2,4-difluoro-6-methoxyphenyl)-6-((4-((S)-3-hydroxypiperidin-1-yl)-5-(1-(2,2,2-trifluoroethyl)-1H-pyrazol-4-yl)pyridin-2-yl)amino)nicotinonitrile

The titled compound (1.2 mg, Yield: 1.7%) was prepared in accordance with the same procedures as in Example 52, using Intermediates A2-6 and C8-2. 1H NMR (CD₃OD, 400 MHz) δ 8.15 (s, 1H), 8.10 (s, 1H), 8.00-7.90 (m, 2H), 7.72 (s, 1H), 7.55 (d, 1H), 6.88 (d, 1H), 6.77 (t, 1H), 5.01 (q, 2H), 3.88 (s, 3H), 3.80-3.70 (m, 1H), 3.30-3.20 (m, 1H), 3.05-2.95 (m, 1H), 2.60-2.55 (m, 1H), 2.50-2.40 (m, 1H), 2.00-1.95 (m, 1H), 1.75-1.65 (m, 1H), 1.65-1.50 (m, 1H), 1.40-1.30 (m, 1H) MS m/z=586.2 (M+H)+

### Example 318: 6-((4-((S)-3-aminopiperidin-1-yl)-5-(1-(2,2,2-trifluoroethyl)-1H-pyrazol-4-yl)pyridin-2-yl)amino)-2-(2-fluoro-6-methoxyphenyl)nicotinonitrile hydrochloride

The titled compound (14.2 mg, Yield: 64.7%) was prepared in accordance with the same procedures as in Example 52, using Intermediates A3-4 and C8-1. 1H NMR (CD₃OD, 400 MHz) δ 8.18 (s, 1H), 8.14 (s, 1H), 8.09 (d, 1H), 7.96 (s, 1H), 7.65-7.45 (m, 3H), 7.05 (d, 1H), 6.94 (t, 1H), 5.05 (q, 2H), 3.89 (s, 3H), 3.60-3.50 (m, 1H), 3.40-3.20 (m, 2H), 2.80-2.55 (m, 2H), 2.20-2.10 (m, 1H), 1.85-1.65 (m, 2H), 1.60-1.45 (m, 1H) MS m/z=567.3 (M+H)+

### Example 319: 6-((4-((S)-3-aminopiperidin-1-yl)-5-(1-(2-hydroxy-2-methylpropyl)-1H-pyrazol-4-yl)pyridin-2-yl)amino)-2-(2-fluoro-6-methoxyphenyl)nicotinonitrile hydrochloride

The titled compound (19.5 mg, Yield: 89.6%) was prepared in accordance with the same procedures as in Example 52, using Intermediates A3-23 and C8-1. 1H NMR (CD₃OD, 400 MHz) δ 8.25-8.15 (m, 1H), 8.09 (s, 1H), 8.00 (s, 1H), 7.81 (s, 1H), 7.56 (q, 1H), 7.50-7.40 (m, 1H), 7.40-7.20 (m, 1H), 7.06 (d, 1H), 6.95 (t, 1H), 4.18 (s, 2H), 3.89 (s, 3H), 3.75-3.60 (m, 1H), 3.45-3.35 (m, 2H), 2.90-2.80 (m, 1H), 2.80-2.65 (m, 1H), 2.20-2.10 (m, 1H), 1.90-1.70 (m, 2H), 1.60-1.45 (m, 1H), 1.25 (s, 3H), 1.23 (s, 3H) MS m/z=557.3 (M+H)+

### Example 320: 6-((4-((S)-3-aminopiperidin-1-yl)-5-(1-(2,2,2-trifluoroethyl)-1H-pyrazol-4-yl)pyridin-2-yl)amino)-2-(2,4-difluoro-6-methoxyphenyl)nicotinonitrile hydrochloride

The titled compound (16.7 mg, Yield: 59.5%) was prepared in accordance with the same procedures as in Example 52, using Intermediates A3-4 and C8-2. 1H NMR (CD₃OD, 400 MHz) δ 8.30-8.25 (m, 1H), 8.11 (s, 1H), 8.00 (s, 1H), 7.93 (s, 1H), 7.50-7.35 (m, 1H), 7.30-7.10 (m, 1H), 6.94 (d, 1H), 6.82 (t, 1H), 5.06 (q, 2H), 3.90 (s, 3H), 3.80-3.65 (m, 1H), 3.50-3.35 (m, 2H), 2.95-2.75 (m, 2H), 2.25-2.15 (m, 1H), 1.90-1.65 (m, 2H), 1.65-1.50 (m, 1H) MS m/z=585.2 (M+H)+

### Example 321: 6-((4-((3R,4R)-3-amino-4-fluoropiperidin-1-yl)-5-(1-(difluoromethyl)-1H-pyrazol-4-yl)pyridin-2-yl)amino)-2-(2-fluoro-6-methoxyphenyl)nicotinonitrile hydrochloride

The titled compound (2.5 mg, Yield: 40.0%) was prepared in accordance with the same procedures as in Example 52, using Intermediates B1-7 and C8-1. 1H NMR (CD₃OD, 400 MHz) δ 8.41 (s, 1H), 8.16 (s, 1H), 8.10 (s, 1H), 7.99 (d, 1H), 7.82 (s, 1H), 7.60-7.50 (m, 2H), 7.55 (t, 1H), 7.03 (d, 1H), 6.93 (t, 1H), 4.45-4.20 (m, 1H), 3.88 (s, 3H), 3.30-3.20 (m, 1H), 3.10-2.95 (m, 1H), 2.75 (q, 1H), 2.48 (t, 1H), 2.10-1.95 (m, 1H), 1.90-1.70 (m, 1H), 1.65-1.50 (m, 1H) MS m/z=553.2 (M+H)+

### Example 322: 6-((4-((3R,4S)-3-amino-4-fluoropiperidin-1-yl)-5-(1-(difluoromethyl)-1H-pyrazol-4-yl)pyridin-2-yl)amino)-2-(2-fluoro-6-methoxyphenyl)nicotinonitrile hydrochloride

The titled compound (2.5 mg, Yield: 40.0%) was prepared in accordance with the same procedures as in Example 52, using Intermediates B1-6 and C8-1. 1H NMR (CD₃OD, 400 MHz) δ 8.51 (s, 1H), 8.31 (d, 1H), 8.16 (s, 1H), 8.09 (s, 1H), 7.65-7.50 (m, 1H), 7.61 (t, 1H), 7.33 (d, 1H), 7.05 (d, 1H), 6.98 (t, 1H), 5.25-5.10 (m, 1H), 3.88 (s, 3H), 3.75-3.60 (m, 1H), 3.55-3.45 (m, 1H), 3.35-3.15 (m, 2H), 2.30-2.10 (m, 2H), 2.10-2.00 (m, 1H) MS m/z=553.2 (M+H)+

### Example 323: 6-((4-((S)-3-aminopiperidin-1-yl)-6′-morpholino-[3,3′-bipyridin]-6-yl)amino)-2-(2-fluoro-6-methoxyphenyl)nicotinonitrile hydrochloride

The titled compound (21.6 mg, Yield: 68.8%) was prepared in accordance with the same procedures as in Example 52, using Intermediates A3-19 and C8-1. 1H NMR (CD₃OD, 400 MHz) δ 8.31 (d, 1H), 8.23 (s, 1H), 8.10-7.95 (m, 2H), 7.59 (q, 1H), 7.40-7.30 (m, 2H), 7.09 (d, 1H), 7.04 (s, 1H), 6.97 (t, 1H), 3.91 (s, 3H), 3.90-3.80 (m, 4H), 3.80-3.60 (m, 4H), 3.60-3.50 (m, 1H), 3.40-3.20 (m, 2H), 3.05-2.90 (m, 2H), 2.25-2.15 (m, 1H), 1.90-1.80 (m, 1H), 1.80-1.65 (m, 1H), 1.65-1.50 (m, 1H) MS m/z=581.3 (M+H)+

### Example 324: N-(4-((3S,5R)-3-amino-5-fluoropiperidin-1-yl)-5-(2-(2,2,2-trifluoroethyl)-1H-pyrazol-4-yl)pyridin-2-yl)-2-(2,4-difluoro-6-methoxyphenyl)pyrimidin-4-amine

The titled compound (6.4 mg, Yield: 57.8%) was prepared in accordance with the same procedures as in Example 52, using Intermediates B3-54 and C1-7. 1H NMR (CDCl₃, 400 MHz) δ 8.63-8.53 (m, 1H), 8.15 (s, 1H), 7.99 (s, 1H), 7.85-7.76 (m, 2H), 7.59 (s, 1H), 7.39 (s, 1H), 6.65-6.51 (m, 2H), 4.87-4.75 (m, 2H), 4.75-4.66 (m, 1H), 4.63-4.54 (m, 1H), 3.89 (s, 3H), 3.44-3.28 (m, 1H), 3.25-3.14 (m, 1H), 3.06-2.93 (m, 1H), 2.88-2.70 (m, 1H), 2.51-2.38 (m, 1H), 2.38-2.23 (m, 1H), 1.63-1.49 (m, 1H) MS m/z=579.2 (M+H)+

### Example 325: 2-(2-fluoro-6-methoxyphenyl)-6-((4-((S)-3-hydroxypiperidin-1-yl)-5-(1-(1-methylpiperidin-4-yl)-1H-pyrazol-4-yl)pyridin-2-yl)amino)nicotinonitrile

The titled compound (17.2 mg, Yield: 23.9%) was prepared in accordance with the same procedures as in Example 52, using Intermediates A2-5 and C8-1. 1H NMR (CD₃OD, 400 MHz) δ 8.10-8.05 (m, 2H), 7.97 (d, 1H), 7.83 (s, 1H), 7.75-7.70 (m, 1H), 7.55-7.45 (m, 2H), 7.02 (d, 1H), 6.91 (t, 1H), 4.30-4.20 (m, 1H), 3.88 (s, 3H), 3.75-3.65 (m, 1H), 3.30-3.20 (m, 1H), 3.10-2.95 (m, 3H), 2.65-2.50 (m, 1H), 2.45-2.35 (m, 1H), 2.31 (s, 3H), 2.30-2.20 (m, 2H), 2.20-2.00 (m, 5H), 2.00-1.90 (m, 1H), 1.75-1.65 (m, 1H), 1.60-1.50 (m, 1H), 1.40-1.30 (m, 1H) MS m/z=584.2 (M+H)+

### Example 326: 6-((4-((3R,4R)-4-amino-3-hydroxypiperidin-1-yl)-5-(1-(difluoromethyl)-1H-pyrazol-4-yl)pyridin-2-yl)amino)-2-(2-fluoro-6-methoxyphenyl)nicotinonitrile

The titled compound (20.1 mg, Yield: 43.1%) was prepared in accordance with the same procedures as in Example 52, using Intermediates B3-52 and C8-1. 1H NMR (CDCl₃, 400 MHz) δ 8.15-8.10 (m, 2H), 7.97 (s, 1H), 7.91 (d, 1H), 7.73 (s, 1H), 7.55-7.35 (m, 3H), 7.26 (t, 1H), 6.95-6.85 (m, 2H), 3.89 (s, 3H), 3.50-3.40 (m, 1H), 3.40-3.25 (m, 1H), 3.25-3.15 (m, 1H), 2.65-2.50 (m, 2H), 2.50-2.40 (m, 1H), 1.85-1.75 (m, 1H), 1.45-1.35 (m, 1H) MS m/z=551.2 (M+H)+

### Example 327: 6-((4-((S)-3-aminopiperidin-1-yl)-5-(1-ethyl-1H-pyrazol-4-yl)pyridin-2-yl)amino)-2-(2-fluoro-6-methoxyphenyl)nicotinonitrile hydrochloride

The titled compound (16.3 mg, Yield: 62.2%) was prepared in accordance with the same procedures as in Example 52, using Intermediates A3-73 and C8-1. 1H NMR (CD₃OD, 400 MHz) δ 8.23 (d, 1H), 8.10-8.00 (m, 2H), 7.80 (s, 1H), 7.58 (q, 1H), 7.39 (d, 1H), 7.20-7.10 (m, 1H), 7.08 (d, 1H), 6.96 (t, 1H), 4.28 (q, 2H), 3.90 (s, 3H), 3.80-3.70 (m, 1H), 3.55-3.45 (m, 1H), 3.45-3.35 (m, 1H), 2.95-2.80 (m, 2H), 2.25-2.15 (m, 1H), 1.90-1.65 (m, 2H), 1.65-1.58 (m, 1H), 1.56 (t, 3H) MS m/z=513.2 (M+H)+

### Example 328: 6-((4-((S)-3-aminopiperidin-1-yl)-5-(1-isopropyl-1H-pyrazol-4-yl)pyridin-2-yl)amino)-2-(2-fluoro-6-methoxyphenyl)nicotinonitrile hydrochloride

The titled compound (33.6 mg, Yield: 99.9%) was prepared in accordance with the same procedures as in Example 52, using Intermediates A3-74 and C8-1. 1H NMR (CD₃OD, 400 MHz) δ 8.23 (d, 1H), 8.10-8.00 (m, 2H), 7.80 (s, 1H), 7.58 (q, 1H), 7.40 (d, 1H), 7.20-7.10 (m, 1H), 7.08 (d, 1H), 6.96 (t, 1H), 4.66-4.59 (m, 1H), 3.90 (s, 3H), 3.80-3.70 (m, 1H), 3.55-3.35 (m, 2H), 2.95-2.80 (m, 2H), 2.25-2.15 (m, 1H), 1.85-1.70 (m, 2H), 1.70-1.60 (m, 1H), 1.56 (d, 6H) MS m/z=527.2 (M+H)+

### Example 329: 6-((4-((S)-3-aminopiperidin-1-yl)-5-(1-cyclopropyl-1H-pyrazol-4-yl)pyridin-2-yl)amino)-2-(2-fluoro-6-methoxyphenyl)nicotinonitrile hydrochloride

The titled compound (24.1 mg, Yield: 93.0%) was prepared in accordance with the same procedures as in Example 52, using Intermediates A3-75 and C8-1. 1H NMR (CD₃OD, 400 MHz) δ 8.21 (d, 1H), 8.10-8.00 (m, 2H), 7.79 (s, 1H), 7.58 (q, 1H), 7.41 (d, 1H), 7.30-7.15 (m, 1H), 7.08 (d, 1H), 6.96 (t, 1H), 3.90 (s, 3H), 3.80-3.70 (m, 2H), 3.50-3.40 (m, 1H), 3.40-3.30 (m, 1H), 2.95-2.75 (m, 2H), 2.25-2.15 (m, 1H), 1.90-1.70 (m, 2H), 1.70-1.50 (m, 1H), 1.20-1.10 (m, 2H), 1.10-1.05 (m, 2H) MS m/z=525.2 (M+H)+

### Example 330: 6-((4-((S)-3-aminopiperidin-1-yl)-5-(1-(cyclopropylmethyl)-1H-pyrazol-4-yl)pyridin-2-yl)amino)-2-(2-fluoro-6-methoxyphenyl)nicotinonitrile hydrochloride

The titled compound (23.4 mg, Yield: 90.5%) was prepared in accordance with the same procedures as in Example 52, using Intermediates A3-76 and C8-1. 1H NMR (CD₃OD, 400 MHz) δ 8.14 (d, 1H), 8.15-8.05 (m, 2H), 7.83 (s, 1H), 7.56 (q, 1H), 7.49 (d, 1H), 7.40-7.30 (m, 1H), 7.06 (d, 1H), 6.95 (t, 1H), 4.15-4.05 (m, 2H), 3.89 (s, 3H), 3.75-3.60 (m, 1H), 3.45-3.35 (m, 2H), 2.90-2.70 (m, 2H), 2.25-2.10 (m, 1H), 1.90-1.70 (m, 2H), 1.65-1.50 (m, 1H), 1.45-1.30 (m, 1H), 0.70-0.62 (m, 2H), 0.50-0.42 (m, 2H) MS m/z=539.2 (M+H)+

### Example 331: 6-((5-(1-(difluoromethyl)-1H-pyrazol-4-yl)-4-((3R,4R)-4-fluoro-3-hydroxypiperidin-1-yl)pyridin-2-yl)amino)-2-(2-fluoro-6-methoxyphenyl)nicotinonitrile

The titled compound (28.2 mg, Yield: 47.7%) was prepared in accordance with the same procedures as in Example 52, using Intermediates B3-53 and C8-1. 1H NMR (CD₃OD, 400 MHz) δ 8.43 (s, 1H), 8.15 (s, 1H), 8.11 (s, 1H), 7.98 (d, 1H), 7.84 (s, 1H), 7.54 (t, 1H), 7.54-7.49 (m, 2H), 7.02 (d, 1H), 6.93 (t, 1H), 4.45-4.25 (m, 1H), 3.88 (s, 3H), 3.80-3.65 (m, 1H), 3.30-3.20 (m, 1H), 3.20-3.10 (m, 1H), 2.80-2.65 (m, 1H), 2.60-2.50 (m, 1H), 2.10-1.95 (m, 1H), 1.80-1.65 (m, 1H) MS m/z=554.2 (M+H)+

### Example 332: N-(4-((3S,5S)-3-amino-5-fluoropiperidin-1-yl)-5-(1-(2,2,2-trifluoroethyl)-1H-pyrazol-4-yl)pyridin-2-yl)-2-(2-fluoro-6-methoxyphenyl)pyrimidin-4-amine hydrochloride

The titled compound (17.4 mg, Yield: 52.7%) was prepared in accordance with the same procedures as in Example 52, using Intermediates B3-55 and C1-1. 1H NMR (CD₃OD, 400 MHz) δ 8.95-8.69 (m, 1H), 8.32-8.11 (m, 2H), 7.99 (s, 1H), 7.82 (s, 1H), 7.66 (s, 1H), 7.51-7.34 (m, 1H), 7.28-7.12 (m, 1H), 7.12-6.92 (m, 1H), 5.26-4.98 (m, 2H), 4.20-4.06 (m, 1H), 4.06 (s, 3H), 3.80-3.60 (m, 1H), 3.49-3.39 (m, 1H), 3.30-3.15 (m, 1H), 2.65-2.42 (m, 1H), 2.15-1.87 (m, 1H) MS m/z=561.2 (M+H)+

### Example 333: N-(4-((3S,5S)-3-amino-5-fluoropiperidin-1-yl)-5-(1-(2,2,2-trifluoroethyl)-1H-pyrazol-4-yl)pyridin-2-yl)-2-(2,4-difluoro-6-methoxyphenyl)pyrimidin-4-amine hydrochloride

The titled compound (28.2 mg, Yield: 47.7%) was prepared in accordance with the same procedures as in Example 52, using Intermediates B3-55 and C1-7. 1H NMR (CD₃OD, 400 MHz) δ 8.90 (s, 1H), 8.31-8.15 (m, 2H), 8.00 (s, 1H), 7.67 (s, 1H), 7.50 (s, 1H), 7.12-6.99 (m, 1H), 6.99-6.83 (m, 1H), 5.23-4.98 (m, 2H), 4.17-4.04 (m, 1H), 4.04 (s, 3H), 3.77-3.62 (m, 1H), 3.48-3.38 (m, 1H), 3.29-3.19 (m, 1H), 2.65-2.48 (m, 1H), 2.14-1.88 (m, 1H) MS m/z=579.2 (M+H)+

### Example 334: 4-((3R,4R)-3-amino-4-fluoropiperidin-1-yl)-5-(1-(difluoromethyl)-1H-pyrazol-4-yl)-N-(6-(2-fluoro-6-methoxyphenyl)-5-nitropyridin-2-yl)pyridin-2-amine hydrochloride

The titled compound (12.1 mg, Yield: 62.7%) was prepared in accordance with the same procedures as in Example 52, using Intermediates B1-7 and C3-1. 1H NMR (CD₃OD, 400 MHz) δ 8.55-8.45 (m, 2H), 8.19 (s, 1H), 8.11 (s, 1H), 7.59 (t, 1H), 7.59-7.40 (m, 3H), 7.00-6.85 (m, 2H), 4.75-4.55 (m, 1H), 3.79 (s, 3H), 3.75-3.60 (m, 1H), 3.55-3.40 (m, 1H), 3.00-2.80 (m, 2H), 2.30-2.15 (m, 1H), 2.10-1.95 (m, 2H) MS m/z=573.2 (M+H)+

### Example 335: N-(4-((3S,5R)-3-amino-5-fluoropiperidin-1-yl)-5-(1-isopropyl-1H-pyrazol-4-yl)pyridin-2-yl)-2-(2,4-difluoro-6-methoxyphenyl)pyrimidin-4-amine

The titled compound (6.0 mg, Yield: 53.1%) was prepared in accordance with the same procedures as in Example 52, using Intermediates B3-56 and C1-7. 1H NMR (CDCl₃, 400 MHz) δ 8.62-8.55 (m, 1H), 8.17 (s, 1H), 7.77 (s, 1H), 7.71 (s, 1H), 7.65 (s, 1H), 7.49 (s, 1H), 7.40-7.32 (m, 1H), 6.63-6.53 (m, 2H), 4.77-4.65 (m, 0.5H), 4.65-4.53 (m, 1.5H), 3.88 (s, 3H), 3.46-3.36 (m, 1H), 3.29-3.20 (m, 1H), 3.04-2.94 (m, 1H), 2.81-2.71 (m, 1H), 2.43-2.28 (m, 2H), 1.63 (d, 6H) MS m/z=539.2 (M+H)+

### Example 336: N-(4-((3S,5S)-3-amino-5-fluoropiperidin-1-yl)-5-(1-isopropyl-1H-pyrazol-4-yl)pyridin-2-yl)-2-(2,4-difluoro-6-methoxyphenyl)pyrimidin-4-amine

The titled compound (12.6 mg, Yield: 51.2%) was prepared in accordance with the same procedures as in Example 52, using Intermediates B3-57 and C1-7. 1H NMR (CDCl₃, 400 MHz) δ 8.60-8.53 (m, 1H), 8.24 (s, 1H), 7.93 (s, 1H), 7.84 (s, 1H), 7.78 (s, 1H), 7.51 (s, 1H), 7.35 (s, 1H), 6.62-6.51 (m, 2H), 4.96-4.89 (m, 0.5H), 4.84-4.76 (m, 0.5H), 4.62-4.50 (m, 1H), 3.88 (s, 3H), 3.58-3.46 (m, 1H), 3.40-3.23 (m, 2H), 2.79-2.69 (m, 0.5H), 2.69-2.60 (m, 0.5H), 2.37-2.25 (m, 2H), 1.63 (d, 6H), MS m/z=539.2 (M+H)+

### Example 337: 6-((4-((3S,4R)-3-amino-4-methylpiperidin-1-yl)-5-(1-(difluoromethyl)-1H-pyrazol-4-yl)pyridin-2-yl)amino)-2-(2-fluoro-6-methoxyphenyl)-nicotinonitrile hydrochloride

The titled compound (20.4 mg, Yield: 54.0%) was prepared in accordance with the same procedures as in Example 52, using Intermediates B1-3 and C8-1. 1H NMR (CD₃OD, 400 MHz) δ 8.47 (s, 1H), 8.28 (d, 1H), 8.12 (s, 1H), 8.06 (s, 1H), 7.60 (t, 1H), 7.60-7.55 (m, 1H), 7.38 (d, 1H), 7.20-7.10 (m, 1H), 7.09 (d, 1H), 6.97 (t, 1H), 3.90 (s, 3H), 3.90-3.80 (m, 1H), 3.60-3.50 (m, 1H), 3.10-3.00 (m, 1H), 3.00-2.80 (m, 2H), 1.90-1.70 (m, 2H), 1.65-1.40 (m, 1H), 1.17 (d, 3H) MS m/z=549.2 (M+H)+

### Example 338: 6-((4-((3S,4R)-3-amino-4-methylpiperidin-1-yl)-5-(1-(difluoromethyl)-1H-pyrazol-4-yl)pyridin-2-yl)amino)-2-(2,4-difluoro-6-methoxyphenyl)nicotinonitrile hydrochloride

The titled compound (19.6 mg, Yield: 57.4%) was prepared in accordance with the same procedures as in Example 52, using Intermediates B1-3 and C8-2. 1H NMR (CD₃OD, 400 MHz) δ 8.48 (s, 1H), 8.26 (d, 1H), 8.13 (s, 1H), 8.06 (s, 1H), 7.60 (t, 1H), 7.40 (d, 1H), 7.20-7.10 (m, 1H), 6.95 (d, 1H), 6.82 (t, 1H), 3.90 (s, 3H), 3.85-3.75 (m, 1H), 3.60-3.50 (m, 1H), 3.10-3.00 (m, 1H), 3.00-2.80 (m, 2H), 1.90-1.70 (m, 2H), 1.65-1.40 (m, 1H), 1.17 (d, 3H) MS m/z=567.2 (M+H)+

### Example 339: N-(4-((3R,4R)-3-amino-4-fluoropiperidin-1-yl)-5-(1-isopropyl-1H-pyrazol-4-yl)pyridin-2-yl)-2-(2,4-difluoro-6-methoxyphenyl)pyrimidin-4-amine hydrochloride

The titled compound (11.2 mg, Yield: 38.0%) was prepared in accordance with the same procedures as in Example 52, using Intermediates B3-58 and C1-7. 1H NMR (CD₃OD, 400 MHz) δ 8.75 (d, 1H), 8.20 (s, 1H), 8.15 (s, 1H), 7.86 (s, 1H), 7.65-7.45 (m, 1H), 7.20 (s, 1H), 6.99 (d, 1H), 6.86 (t, 1H), 4.90-4.70 (m, 1H), 4.70-4.60 (m, 1H), 4.00-3.85 (m, 4H), 3.75-3.65 (m, 1H), 3.65-3.50 (m, 1H), 3.15-2.95 (m, 2H), 2.30-2.20 (m, 1H), 2.00-1.85 (m, 1H), 1.58 (d, 6H) MS m/z=539.2 (M+H)+

### Example 340: 6-((4-((3R,4R)-3-amino-4-fluoropiperidin-1-yl)-5-(1-isopropyl-1H-pyrazol-4-yl)pyridin-2-yl)amino)-2-(2,4-difluoro-6-methoxyphenyl)nicotinonitrile hydrochloride

The titled compound (10.0 mg, Yield: 51.6%) was prepared in accordance with the same procedures as in Example 52, using Intermediates B3-58 and C8-2. 1H NMR (CD₃OD, 400 MHz) δ 8.25 (d, 1H), 8.15-8.05 (m, 2H), 7.82 (s, 1H), 7.45-7.35 (m, 1H), 7.25-7.10 (m, 1H), 6.95 (d, 1H), 6.83 (t, 1H), 4.85-4.65 (m, 1H), 4.65-4.60 (m, 1H), 3.97-3.82 (m, 4H), 3.70-3.60 (m, 1H), 3.60-3.45 (m, 1H), 3.10-2.90 (m, 2H), 2.30-2.20 (m, 1H), 2.00-1.80 (m, 1H), 1.57 (d, 6H) MS m/z=563.2 (M+H)+

### Example 341: 4-((3R,4R)-3-amino-4-fluoropiperidin-1-yl)-5-(1-(difluoromethyl)-1H-pyrazol-4-yl)-N-(5-fluoro-6-(2-fluoro-6-methoxyphenyl)pyridin-2-yl)pyridin-2-amine hydrochloride

The titled compound (23.6 mg, Yield: 67.9%) was prepared in accordance with the same procedures as in Example 52, using Intermediates B1-7 and C1-2. 1H NMR (CD₃OD, 400 MHz) δ 8.51 (s, 1H), 8.15-8.05 (m, 2H), 7.87 (t, 1H), 7.61 (t, 1H), 7.61-7.52 (m, 1H), 7.34 (d, 1H), 7.06 (d, 1H), 7.05-6.95 (m, 1H), 6.93 (t, 1H), 4.85-4.65 (m, 1H), 3.88 (s, 3H), 3.88-3.80 (m, 1H), 3.65-3.50 (m, 2H), 3.10-2.90 (m, 2H), 2.30-2.20 (m, 1H), 2.00-1.80 (m, 1H) MS m/z=546.2 (M+H)+

### Test Example: Evaluation of Inhibitory Activity Against Human Recombinant HPK1

The inhibitory activity against human recombinant HPK1 was evaluated using the Time-Resolved Fluorescence Resonance Energy Transfer (TR-FRET) technology of Perkin-Elmer. The test compound was prepared as a 20 mM stock solution with dimethyl sulfoxide (DMSO), serially diluted in a buffer solution, and used at final compound concentrations of 0.32 to 1000 nM. The evaluation of inhibitory activity was carried out at room temperature, using recombinant human HPK1 (BPS Bioscience). Briefly, recombinant human HPK1, serially diluted each test compound, ULight-p70S6K peptide substrate (PerkinElmer), and ATP at each concentration (Km and final concentration) were added to a kinase buffer solution (50 mM HEPES pH 7.4, 10 mM MgCl₂, 1 mM EGTA, 3 mM MnCl₂, 2 mM DTT, and 0.01% Tween-20). The resulting mixture was added to each well of a 384-well plate (Optiplate™ 384, white, PerkinElmer). The enzyme reaction was carried out at room temperature for 1 hour. After the reaction was suspended by adding 4 μL of a stop solution (10 mM EDTA) thereto, the mixture was stirred for 1 minute and then a detection solution containing Europium-labeled anti-phosphopeptide antibody (final concentration: 2 nM, PerkinElmer) was added thereto. The signals of TR-FRET at excitation wavelength (320 nm) and emission wavelength (665 nm) were detected using a microplate reader (Envision Multilabel Reader, Perkin-Elmer). Inhibition by the test compound was measured as % inhibition in the signals, compared to the control group treated with DMSO only. The IC₅₀ value of each compound was calculated using the GraphPad Prism program (GraphPad Software, Inc., San Diego, CA, USA). The IC₅₀ values of test compounds for inhibitory activity against HPK1 were classified as follows:
A: IC₅₀≤10 nM
B: 10 nM<IC₅₀≤50 nM
C: 50 nM<IC₅₀≤100 nM
D: IC₅₀>100 nM

The results are shown in Tables 1 to 3 below.

TABLE 1

| Example | IC₅₀ |
|---|---|
| 1 | B |
| 2 | B |
| 3 | C |
| 4 | C |
| 5 | B |
| 6 | B |
| 7 | A |
| 8 | B |
| 9 | B |
| 10 | B |
| 11 | A |
| 12 | A |
| 13 | A |
| 14 | A |
| 15 | A |
| 16 | A |
| 17 | A |
| 18 | A |
| 19 | A |
| 20 | A |
| 21 | B |
| 22 | A |
| 23 | B |
| 24 | A |
| 25 | B |
| 26 | C |
| 27 | B |
| 28 | B |
| 29 | A |
| 30 | B |
| 31 | A |
| 32 | B |
| 33 | B |
| 34 | B |
| 35 | A |
| 36 | A |
| 37 | B |
| 38 | A |
| 39 | A |
| 40 | A |
| 41 | A |
| 42 | B |
| 43 | A |
| 44 | B |
| 45 | D |
| 46 | A |
| 47 | A |
| 48 | A |
| 49 | D |
| 50 | D |
| 51 | C |
| 52 | A |
| 53 | A |
| 54 | B |
| 55 | B |
| 56 | B |
| 57 | C |
| 58 | D |
| 59 | A |
| 60 | A |
| 61 | B |
| 62 | C |
| 63 | C |
| 64 | A |
| 65 | A |
| 66 | A |
| 67 | A |
| 68 | B |
| 69 | A |
| 70 | A |
| 71 | A |
| 72 | A |
| 73 | A |
| 74 | A |
| 75 | A |
| 76 | A |
| 77 | A |
| 78 | A |
| 79 | A |
| 80 | A |
| 81 | D |
| 82 | A |
| 83 | A |
| 84 | B |
| 85 | A |
| 86 | C |
| 87 | A |
| 88 | A |
| 89 | A |
| 90 | A |
| 91 | A |
| 92 | A |
| 93 | B |
| 94 | A |
| 95 | A |
| 96 | A |
| 97 | A |
| 98 | A |
| 99 | B |
| 100 | A |
| 101 | A |
| 102 | A |
| 103 | A |
| 104 | A |
| 105 | A |
| 106 | A |
| 107 | A |
| 108 | A |
| 109 | A |
| 110 | A |
| 111 | A |
| 112 | A |
| 113 | B |
| 114 | A |
| 115 | A |
| 116 | A |
| 117 | A |
| 118 | A |
| 119 | A |
| 120 | A |
| 121 | A |
| 122 | A |
| 123 | B |
| 124 | A |
| 125 | A |
| 126 | B |
| 127 | B |
| 128 | D |

TABLE 2

| Example | IC₅₀ |
|---|---|
| 129 | A |
| 130 | A |
| 131 | C |
| 132 | A |
| 133 | A |
| 134 | A |
| 135 | A |
| 136 | A |
| 137 | A |
| 138 | C |
| 139 | A |
| 140 | A |
| 141 | D |
| 142 | A |
| 143 | A |
| 144 | A |
| 145 | A |
| 146 | A |
| 147 | A |
| 148 | A |
| 149 | A |
| 150 | A |
| 151 | A |
| 152 | A |
| 153 | A |
| 154 | A |
| 155 | A |
| 156 | A |
| 157 | A |
| 158 | A |
| 159 | A |
| 160 | A |
| 161 | A |
| 162 | A |
| 163 | A |
| 164 | A |
| 165 | A |
| 166 | A |
| 167 | A |
| 168 | A |
| 169 | A |
| 170 | A |
| 171 | A |
| 172 | A |
| 173 | A |
| 174 | A |
| 175 | A |
| 176 | A |
| 177 | A |
| 178 | A |
| 179 | A |
| 180 | A |
| 181 | A |
| 182 | A |
| 183 | A |
| 184 | A |
| 185 | A |
| 186 | A |
| 187 | A |
| 188 | B |
| 189 | A |
| 190 | A |
| 191 | A |
| 192 | B |
| 193 | A |
| 194 | A |
| 195 | A |
| 196 | A |
| 197 | A |
| 198 | A |
| 199 | A |
| 200 | A |
| 201 | A |
| 202 | A |
| 203 | A |
| 204 | A |
| 205 | A |
| 206 | A |
| 207 | A |
| 208 | A |
| 209 | A |
| 210 | A |
| 211 | A |
| 212 | A |
| 213 | A |
| 214 | A |
| 215 | A |
| 216 | A |
| 217 | A |
| 218 | A |
| 219 | A |
| 220 | A |
| 221 | A |
| 222 | A |
| 223 | A |
| 224 | A |
| 225 | A |
| 226 | A |
| 227 | A |
| 228 | B |
| 229 | A |
| 230 | A |
| 231 | A |
| 232 | A |
| 233 | A |
| 234 | A |
| 235 | A |
| 236 | A |
| 237 | A |
| 238 | A |
| 239 | A |
| 240 | A |
| 241 | A |
| 242 | A |
| 243 | A |
| 244 | A |
| 245 | A |
| 246 | A |
| 247 | A |
| 248 | A |
| 249 | A |
| 250 | A |
| 251 | A |
| 252 | A |
| 253 | A |
| 254 | A |
| 255 | A |
| 256 | A |

TABLE 3

| Example | IC₅₀ |
|---|---|
| 257 | A |
| 258 | A |
| 259 | A |
| 260 | A |
| 261 | A |
| 262 | A |
| 263 | A |
| 264 | A |
| 265 | A |
| 266 | A |
| 267 | A |
| 268 | A |
| 269 | A |
| 270 | A |
| 271 | A |
| 272 | A |
| 273 | A |
| 274 | A |
| 275 | A |
| 276 | A |
| 277 | A |
| 278 | A |
| 279 | A |
| 280 | A |
| 281 | A |
| 282 | A |
| 283 | A |
| 284 | A |
| 285 | A |
| 286 | A |
| 287 | A |
| 288 | A |
| 289 | A |
| 290 | A |
| 291 | A |
| 292 | A |
| 293 | A |
| 294 | A |
| 295 | A |
| 296 | A |
| 297 | A |
| 298 | A |
| 299 | A |
| 300 | A |
| 301 | A |
| 302 | A |
| 303 | A |
| 304 | A |
| 305 | A |
| 306 | A |
| 307 | A |
| 308 | A |
| 309 | A |
| 310 | A |
| 311 | A |
| 312 | A |
| 313 | A |
| 314 | A |
| 315 | A |
| 316 | A |
| 317 | A |
| 318 | A |
| 319 | A |
| 320 | A |
| 321 | A |
| 322 | A |
| 323 | A |
| 324 | A |
| 325 | A |
| 326 | A |
| 327 | A |
| 328 | A |
| 329 | A |
| 330 | A |
| 331 | A |
| 332 | A |
| 333 | A |
| 334 | A |
| 335 | A |
| 336 | A |
| 337 | A |
| 338 | A |
| 339 | A |
| 340 | A |
| 341 | A |

From the results in Tables 1 to 3, it can be seen that the compounds according to the present invention have excellent inhibitory activity against HPK1.

1.-14. (canceled)

15. A compound of Formula 1a or a pharmaceutically acceptable salt thereof: 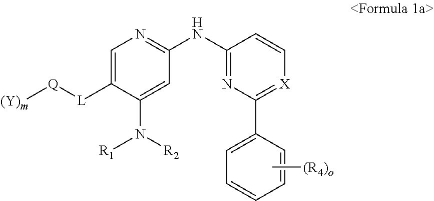
wherein,
R₁ and R₂ form a heterocycloalkyl together with the nitrogen atom to which they are bonded, wherein the heterocycloalkyl is optionally substituted with one or two groups selected from the group consisting of halogen, amino, hydroxy, C₁-C₆ alkyl, C₁-C₆ alkoxy, C₁-C₆ alkyl substituted with halogen, C₁-C₆ alkyl substituted with hydroxy, C₃-C₆ cycloalkyl, C₃-C₆ cycloalkyl substituted with hydroxy, mono- or di-C₁-C₆ alkylamino, aminocarbonyl, C₁-C₆ alkylcarbonylamino, and mono- or di-C₁-C₆ alkylamino-C₁-C₆ alkyl,
X is —C(R₃)═ or —N═,
R₃ is hydrogen, halogen, cyano, nitro, amino, C₁-C₃ alkyl, C₁-C₃ alkyl substituted with halogen, hydroxycarbonyl, C₁-C₃ alkoxycarbonyl, aminocarbonyl, mono- or di-C₁-C₃ alkylaminocarbonyl, or C₁-C₃ alkylsulfonyl,
R₄ is, independently each other, halogen, amino, hydroxy, C₁-C₆ alkyl, C₁-C₆ alkyl substituted with hydroxy, C₁-C₆ alkoxy, C₃-C₆ cycloalkyl, aldehyde, mono- or di-C₁-C₃ alkylamino, mono- or di-C₁-C₃ alkylamino-C₁-C₆ alkyl, C₁-C₆ alkoxycarbonyl, mono- or di-C₁-C₆ alkylaminocarbonyl, C₁-C₆ alkylaminocarbonyl substituted with halogen, C₃-C₆ cycloalkylaminocarbonyl, C₃-C₆ cycloalkylaminocarbonyl substituted with halogen, C₃-C₆ cycloalkylaminocarbonyl substituted with hydroxy, heterocycloalkyl-C₁-C₃ alkyl optionally substituted with hydroxy, or heterocycloalkylcarbonyl optionally substituted with halogen,
is 0, 1, 2, or 3,
L is a bond, —C(O)—, —(CH₂)ₙ—, —C(O)NH—, or —C≡C—,
n is 1, 2, or 3,
Q is a bond; C₃-C₆ cycloalkyl; a 3- to 12-membered heterocycloalkyl having one to three heteroatoms selected from N, O and S; a 5- to 12-membered heteroaryl having one to three heteroatoms selected from N, O and S; or a 5- to 12-membered aryl,
m is 1, 2, or 3,
Y is, independently each other, hydrogen, hydroxy, halogen, amino, C₁-C₆ alkyl, C₁-C₆ alkyl substituted with hydroxy, C₁-C₆ alkyl substituted with halogen, C₁-C₆ alkyl substituted with mono- or di-C₁-C₃ alkylamino, C₁-C₆ alkoxy, C₁-C₆ alkoxy substituted with C₁-C₃ alkoxy, C₁-C₆ alkoxy substituted with halogen, C₁-C₆ alkoxy substituted with mono- or di-C₁-C₃ alkylamino, C₁-C₆ alkoxy-C₁-C₆ alkyl, C₃-C₆ cycloalkyl, C₃-C₆ cycloalkyl-C₁-C₃ alkyl, C₃-C₆ cycloalkyl-C₁-C₃ alkyl substituted with halogen, C₁-C₃ alkylcarbonyl, C₃-C₆ cycloalkyl-aminocarbonyl substituted with hydroxy, mono- or di-C₁-C₃ alkylamino, C₁-C₆ alkylamino substituted with mono- or di-C₁-C₃ alkylamino, aminocarbonyl-C₁-C₆ alkyl, C₁-C₃ alkoxycarbonyl-C₁-C₆ alkyl, C₁-C₃ alkylcarbonylamino, mono- or di-C₁-C₆ alkylaminocarbonyl, mono- or di-C₁-C₆ alkylaminocarbonylamino, or mono- or di-C₁-C₆ alkylaminocarbonyl-C₁-C₆ alkyl, or a heterocycloalkyl-containing group selected from the group consisting of azetidinyl; azetidinyl-C₁-C₆ alkyl; tetrahydropyranyl; tetrahydrofuranyl; pyrrolidinyl;
pyrrolidinyl-C₁-C₃ alkyl; piperidinyl; piperidinyl-C₁-C₃ alkyl; piperidinylcarbonyl;
piperidinyloxy; piperazinyl; piperazinyl-C₁-C₃ alkyl; piperazinylcarbonyl; oxetanyl;
morpholinyl; morpholinyl-C₁-C₃ alkyl; and morpholinylcarbonyl, wherein the heterocycloalkyl of the heterocycloalkyl-containing group is optionally substituted with one to four groups selected from the group consisting of halogen, hydroxy, amino, oxo, C₁-C₃ alkyl, C₁-C₃ alkyl substituted with hydroxy, and mono- or di-C₁-C₃ alkylamino.

16. The compound or pharmaceutically acceptable salt thereof as claimed in claim 15, wherein R₁ and R₂ form piperidine, azetidine, pyrrolidine, piperazine, or 2,8-diazaspiro[4.5]decane together with the nitrogen atom to which they are bonded.

17. The compound or pharmaceutically acceptable salt thereof as claimed in claim 15, wherein X is —N═.

18. The compound or pharmaceutically acceptable salt thereof as claimed in claim 15, wherein X is —C(R₃)═ and R₃ is H, —F, —Cl, —CH₃, —CN, —NO₂, —NH₂, —C(O)OH, —C(O)OCH₃, —C(O)NH₂, —C(O)N(CH₃)₂, or —SO₂CH₃.

19. The compound or pharmaceutically acceptable salt thereof as claimed in claim 15, wherein R₄ is, independently each other, halogen, amino, hydroxy, C₁-C₆ alkyl, C₁-C₆ alkyl substituted with hydroxy, C₁-C₆ alkoxy, aldehyde, mono- or di-C₁-C₃ alkylamino-C₁-C₆ alkyl, C₁-C₆ alkoxycarbonyl, mono- or di-C₁-C₆ alkylaminocarbonyl, azetidinyl-C₁-C₃ alkyl substituted with hydroxy, azetidinylcarbonyl optionally substituted with one or two halogens, or pyrrolidinylcarbonyl optionally substituted with one or two halogens.

20. The compound or pharmaceutically acceptable salt thereof as claimed in claim 15, wherein Q is a bond, C₃-C₆ cycloalkyl, tetrahydropyranyl, pyrrolidinyl, phenyl, pyridinyl, pyrazolyl, thiazolyl, imidazo[1,2-a]pyridinyl, [1,2,4]triazolo[4,3-a]pyridin-6-yl, 2H-benzo[b][1,4]oxazin-3(4H)-onyl, 3,4-dihydroquinolin-2(1H)-onyl, 3,4-dihydro-2H-benzo[b][1,4]oxazinyl, 3,4-dihydro-2H-pyrido[3,2-b][1,4]oxazinyl, 2H-pyrido[3,2-b]-1,4-oxazin-3(4H)-onyl, 1,4-dihydro-2H-benzo[d][1,3]oxazin-2-onyl, or 3,4-dihydro-1,8-naphthyridin-2(1H)-onyl.

21. The compound or pharmaceutically acceptable salt thereof as claimed in claim 15, wherein Y is, independently each other, hydrogen, hydroxy, halogen, amino, C₁-C₆ alkyl, C₁-C₆ alkyl substituted with hydroxy, C₁-C₆ alkyl substituted with halogen, C₁-C₆ alkyl substituted with mono- or di-C₁-C₃ alkylamino, C₁-C₆ alkoxy, C₁-C₆ alkoxy substituted with C₁-C₃ alkoxy, C₁-C₆ alkoxy substituted with halogen, C₁-C₆ alkoxy substituted with mono- or di-C₁-C₃ alkylamino, C₁-C₆ alkoxy-C₁-C₆ alkyl, C₃-C₆ cycloalkyl, C₃-C₆ cycloalkyl-C₁-C₃ alkyl, C₃-C₆ cycloalkyl-C₁-C₃ alkyl substituted with halogen, C₁-C₃ alkylcarbonyl, C₃-C₆ cycloalkyl-aminocarbonyl substituted with hydroxy, mono- or di-C₁-C₃ alkylamino, C₁-C₆ alkylamino substituted with mono- or di-C₁-C₃ alkylamino, aminocarbonyl-C₁-C₆ alkyl, C₁-C₃ alkoxycarbonyl-C₁-C₆ alkyl, C₁-C₃ alkylcarbonylamino, mono- or di-C₁-C₆ alkylaminocarbonyl, mono- or di-C₁-C₆ alkylaminocarbonylamino, or mono- or di-C₁-C₆ alkylaminocarbonyl-C₁-C₆ alkyl.

22. The compound or pharmaceutically acceptable salt thereof as claimed in claim 15, wherein one of Y is a heterocycloalkyl-containing group selected from the group consisting of azetidinyl; azetidinylmethyl; tetrahydropyranyl; tetrahydrofuranyl; pyrrolidinyl; pyrrolidinylmethyl; pyrrolidinylethyl; piperidinyl; piperidinylmethyl; piperidinylcarbonyl; piperidinyloxy; piperazinyl; piperazinylmethyl; piperazinylcarbonyl; oxetanyl; morpholinyl; morpholinylmethyl; morpholinylethyl; and morpholinylcarbonyl, and the remaining Y is hydrogen.

23. The compound or pharmaceutically acceptable salt thereof as claimed in claim 22, wherein the heterocycloalkyl of the heterocycloalkyl-containing group is substituted with one to four groups selected from the group consisting of halogen, hydroxy, amino, oxo, C₁~C₃ alkyl, C₁~C₃ alkyl substituted with hydroxy, and mono- or di-C₁~C₃ alkylamino.

24. A compound or a pharmaceutically acceptable salt thereof, which is selected from the group consisting of:
(S)-cyclopropyl(4-(3-hydroxypiperidin-1-yl)-6-((2-(5-methylpyridin-3-yl)pyrimidin-4-yl)amino)pyridin-3-yl)methanone;
(S)-(6-((2-(5-aminopyridin-3-yl)pyrimidin-4-yl)amino)-4-(3-hydroxypiperidin-1-yl)pyridin-3-yl)(cyclopropyl)methanone;
(S)-cyclopropyl(4-(3-hydroxypiperidin-1-yl)-6-((2-(5-methoxypyridin-3-yl)pyrimidin-4-yl)amino)pyridin-3-yl)methanone;
(S)-cyclopropyl(6-((2-(3-hydroxyphenyl)pyrimidin-4-yl)amino)-4-(3-hydroxypiperidin-1-yl)pyridin-3-yl)methanone;
(S)-cyclopropyl(6-((2-(2-fluoro-6-methoxyphenyl)pyrimidin-4-yl)amino)-4-(3-hydroxypiperidin-1-yl)pyridin-3-yl)methanone;
cyclopropyl(6-((2-(2-fluoro-6-methoxyphenyl)pyrimidin-4-yl)amino)-4-(3-hydroxyazetidin-1-yl)pyridin-3-yl)methanone;
(S)-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-ol;
(S)-1-(2-((2-(2-fluoro-6-methoxyphenyl)pyrimidin-4-yl)amino)-5-((1-(trifluoromethyl)-1H-pyrazol-4-yl)ethynyl)pyridin-4-yl)piperidin-3-ol;
(R)-1-(2-((2-(2-fluoro-6-methoxyphenyl)pyrimidin-4-yl)amino)-5-((1-(trifluoromethyl)-1H-pyrazol-4-yl)ethynyl)pyridin-4-yl)piperidin-3-ol;
1-(2-((2-(2-fluoro-6-methoxyphenyl)pyrimidin-4-yl)amino)-5-((tetrahydro-2H-pyran-4-yl)ethynyl)pyridin-4-yl)azetidin-3-ol;
(S)-1-(2-((2-(2-fluoro-6-methoxyphenyl)pyrimidin-4-yl)amino)-5-((tetrahydro-2H-pyran-4-yl)ethynyl)pyridin-4-yl)piperidin-3-ol;
(S)-1-(2-((2-(2-fluoro-6-methoxyphenyl)pyrimidin-4-yl)amino)-5-(1-methyl-1H-pyrazol-4-yl)pyridin-4-yl)piperidin-3-ol;
(S)-1-(2-((2-(2-fluoro-6-methoxyphenyl)pyrimidin-4-yl)amino)-5-(1-isopropyl-1H-pyrazol-4-yl)pyridin-4-yl)piperidin-3-ol;
(3S)-1-(2-((2-(2-fluoro-6-methoxyphenyl)pyrimidin-4-yl)amino)-5-(1-(tetrahydrofuran-3-yl)-1H-pyrazol-4-yl)pyridin-4-yl)piperidin-3-ol;
(S)-1-(2-((2-(2-fluoro-6-methoxyphenyl)pyrimidin-4-yl)amino)-5-(1-(1-methylpiperidin-4-yl)-1H-pyrazol-4-yl)pyridin-4-yl)piperidin-3-ol;
(S)-1-(2-((2-(2-fluoro-6-methoxyphenyl)pyrimidin-4-yl)amino)-5-(1-(2,2,2-trifluoroethyl)-1H-pyrazol-4-yl)pyridin-4-yl)piperidin-3-ol;
(S)-1-(5-(1-(cyclopropylmethyl)-1H-pyrazol-4-yl)-2-((2-(2-fluoro-6-methoxyphenyl)pyrimidin-4-yl)amino)pyridin-4-yl)piperidin-3-ol;
(S)-1-(2-((2-(2-fluoro-6-methoxyphenyl)pyrimidin-4-yl)amino)-5-(4-(tetrahydro-2H-pyran-4-yl)phenyl)pyridin-4-yl)piperidin-3-ol;
(S)-1-(2-((2-(2-fluoro-6-methoxyphenyl)pyrimidin-4-yl)amino)-5-(4-(4-methylpiperazin-1-yl)phenyl)pyridin-4-yl)piperidin-3-ol;
(S)-1-(6-((2-(2-fluoro-6-methoxyphenyl)pyrimidin-4-yl)amino)-6′-morpholino-[3,3′-bipyridin]-4-yl)piperidin-3-ol;
(S)-1-(2-((2-(4-(hydroxymethyl)thiophen-2-yl)pyrimidin-4-yl)amino)-5-(1-(tetrahydro-2H-pyran-4-yl)-1H-pyrazol-4-yl)pyridin-4-yl)piperidin-3-ol;
(S)-1-(2-((2-(2,6-difluorophenyl)pyrimidin-4-yl)amino)-5-(1-(tetrahydro-2H-pyran-4-yl)-1H-pyrazol-4-yl)pyridin-4-yl)piperidin-3-ol;
(S)-1-(2-((2-(3,6-difluoro-2-methoxyphenyl)pyrimidin-4-yl)amino)-5-(1-(tetrahydro-2H-pyran-4-yl)-1H-pyrazol-4-yl)pyridin-4-yl)piperidin-3-ol;
(S)-1-(2-((2-(2,3-difluoro-6-methoxyphenyl)pyrimidin-4-yl)amino)-5-(1-(tetrahydro-2H-pyran-4-yl)-1H-pyrazol-4-yl)pyridin-4-yl)piperidin-3-ol;
(S)-1-(2-((2-(2-ethoxy-6-fluorophenyl)pyrimidin-4-yl)amino)-5-(1-(tetrahydro-2H-pyran-4-yl)-1H-pyrazol-4-yl)pyridin-4-yl)piperidin-3-ol;
(S)-1-(2-((2-([1,2,4]triazolo[1,5-a]pyridin-7-yl)pyrimidin-4-yl)amino)-5-(1-(tetrahydro-2H-pyran-4-yl)-1H-pyrazol-4-yl)pyridin-4-yl)piperidin-3-ol;
(S)-1-(2-((2-(pyrazolo[1,5-a]pyridin-3-yl)pyrimidin-4-yl)amino)-5-(1-(tetrahydro-2H-pyran-4-yl)-1H-pyrazol-4-yl)pyridin-4-yl)piperidin-3-ol;
(S)-1-(2-((2-(quinoxalin-6-yl)pyrimidin-4-yl)amino)-5-(1-(tetrahydro-2H-pyran-4-yl)-1H-pyrazol-4-yl)pyridin-4-yl)piperidin-3-ol;
(S)-1-(2-((2-(8-fluoroquinolin-7-yl)pyrimidin-4-yl)amino)-5-(1-(tetrahydro-2H-pyran-4-yl)-1H-pyrazol-4-yl)pyridin-4-yl)piperidin-3-ol;
(S)-1-(6-((2-(2-fluoro-6-methoxyphenyl)pyrimidin-4-yl)amino)-6′-((4-methylpiperazin-1-yl)methyl)-[3,3′-bipyridin]-4-yl)piperidin-3-ol;
(S)-1-(6-((2-(2-fluoro-6-methoxyphenyl)pyrimidin-4-yl)amino)-6′-(tetrahydro-2H-pyran-4-yl)-[3,3′-bipyridin]-4-yl)piperidin-3-ol;
(S)-1-(6-((2-(2-fluoro-6-methoxyphenyl)pyrimidin-4-yl)amino)-6′-(morpholinomethyl)-[3,3′-bipyridin]-4-yl)piperidin-3-ol;
(S)-1-(6-((2-(2-fluoro-6-methoxyphenyl)pyrimidin-4-yl)amino)-6′-((4-hydroxypiperidin-1-yl)methyl)-[3,3′-bipyridin]-4-yl)piperidin-3-ol;
1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-carboxamide;
(S)-1-(2-((6-(2-fluoro-6-methoxyphenyl)pyridin-2-yl)amino)-5-(1-(tetrahydro-2H-pyran-4-yl)-1H-pyrazol-4-yl)pyridin-4-yl)piperidin-3-ol;
(S)-(6′-((2-(2-fluoro-6-methoxyphenyl)pyrimidin-4-yl)amino)-4′-(3-hydroxypiperidin-1-yl)-[3,3′-bipyridin]-6-yl)(4-methylpiperazin-1-yl)methanone;
(S)-(6′-((2-(2-fluoro-6-methoxyphenyl)pyrimidin-4-yl)amino)-4′-(3-hydroxypiperidin-1-yl)-[3,3′-bipyridin]-6-yl)(morpholino)methanone;
(S)-(6′-((2-(2-fluoro-6-methoxyphenyl)pyrimidin-4-yl)amino)-4′-(3-hydroxypiperidin-1-yl)-[3,3′-bipyridin]-6-yl)(4-hydroxypiperidin-1-yl)methanone;
(S)-1-(6-((2-(2-fluoro-6-methoxyphenyl)pyrimidin-4-yl)amino)-6′-(((S)-3-fluoropyrrolidin-1-yl)methyl)-[3,3′-bipyridin]-4-yl)piperidin-3-ol;
(3S)-1-(2-((5-fluoro-6-(2-fluoro-6-methoxyphenyl)pyridin-2-yl)amino)-5-(1-(tetrahydro-2H-pyran-4-yl)-1H-pyrazol-4-yl)pyridin-4-yl)piperidin-3-ol;
(3S)-1-(2-((6-(2-fluoro-6-methoxyphenyl)-5-nitropyridin-2-yl)amino)-5-(1-(tetrahydro-2H-pyran-4-yl)-1H-pyrazol-4-yl)pyridin-4-yl)piperidin-3-ol;
(S)-1-(6-((2-(2-fluoro-6-methoxyphenyl)pyrimidin-4-yl)amino)-[3,3′-bipyridin]-4-yl)piperidin-3-ol;
(3S)-1-(6-((2-(2-fluoro-6-methoxyphenyl)pyrimidin-4-yl)amino)-6′-((3-hydroxypyrrolidin-1-yl)methyl)-[3,3′-bipyridin]-4-yl)piperidin-3-ol;
N-(4-(3-(dimethylamino)pyrrolidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)-3-methylpyrrolidin-3-yl)methanol;
N-(4-(4-((dimethylamino)methyl)-4-fluoropiperidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
(4-(2,2-difluoroethyl)-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-4-yl)methanol;
(3S)-1-(6′-((3-(dimethylamino)pyrrolidin-1-yl)methyl)-6-((2-(2-fluoro-6-methoxyphenyl)pyrimidin-4-yl)amino)-[3,3′-bipyridin]-4-yl)piperidin-3-ol;
1-((6′-((2-(2-fluoro-6-methoxyphenyl)pyrimidin-4-yl)amino)-4′-((S)-3-hydroxypiperidin-1-yl)-[3,3′-bipyridin]-6-yl)methyl)piperidin-3-ol;
(S)-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-carboxamide;
(R)-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-carboxamide;
(S)-N-(4-(3-aminopiperidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
(R)-N-(4-(3-aminopiperidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
(6′-((6-(2-fluoro-6-methoxyphenyl)-5-nitropyridin-2-yl)amino)-4′-((S)-3-hydroxypiperidin-1-yl)-[3,3′-bipyridin]-6-yl)(4-hydroxypiperidin-1-yl)methanone;
(S)-1-(6′-((4-aminopiperidin-1-yl)methyl)-6-((2-(2-fluoro-6-methoxyphenyl)pyrimidin-4-yl)amino)-[3,3′-bipyridin]-4-yl)piperidin-3-ol;
(S)-1-(6′-ethoxy-6-((2-(2-fluoro-6-methoxyphenyl)pyrimidin-4-yl)amino)-[3,3′-bipyridin]-4-yl)piperidin-3-ol;
(S)-1-(6-((2-(2-fluoro-6-methoxyphenyl)pyrimidin-4-yl)amino)-6′-(trifluoromethyl)-[3,3′-bipyridin]-4-yl)piperidin-3-ol;
(3S)-1-(2-((6-(2-fluoro-6-methoxyphenyl)-5-methylpyridin-2-yl)amino)-5-(1-(tetrahydro-2H-pyran-4-yl)-1H-pyrazol-4-yl)pyridin-4-yl)piperidin-3-ol;
2-(2-fluoro-6-methoxyphenyl)-6-((4-((S)-3-hydroxypiperidin-1-yl)-5-(1-(tetrahydro-2H-pyran-4-yl)-1H-pyrazol-4-yl)pyridin-2-yl)amino)nicotinonitrile;
methyl 2-(2-fluoro-6-methoxyphenyl)-6-((4-((S)-3-hydroxypiperidin-1-yl)-5-(1-(tetrahydro-2H-pyran-4-yl)-1H-pyrazol-4-yl)pyridin-2-yl)amino)nicotinate;
(S)-1-(2-((2-(2-fluoro-6-methoxyphenyl)pyrimidin-4-yl)amino)-5-(imidazo[1,2-a]pyridin-6-yl)pyridin-4-yl)piperidin-3-ol;
(S)-1-(5-([1,2,4]triazolo[4,3-a]pyridin-6-yl)-2-((2-(2-fluoro-6-methoxyphenyl)pyrimidin-4-yl)amino)pyridin-4-yl)piperidin-3-ol;
2-(2-fluoro-6-methoxyphenyl)-6-((4-((S)-3-hydroxypiperidin-1-yl)-5-(1-(1-methylpiperidin-4-yl)-1H-pyrazol-4-yl)pyrimidin-2-yl)amino)nicotinonitrile;
N-(4-(4-(2-(dimethylamino)ethyl)piperidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
1-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-4-yl)cyclopropan-1-ol;
1-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-4-yl)ethan-1-ol;
2-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-4-yl)-2-methylpropan-1-ol;
2-(2-fluoro-6-methoxyphenyl)-N-(4-(4-methylpiperazin-1-yl)-5-(1-(tetrahydro-2H-pyran-4-yl)-1H-pyrazol-4-yl)pyridin-2-yl)pyrimidin-4-amine;
(S)-1-(2-((2-(7-fluoroquinoxalin-6-yl)pyrimidin-4-yl)amino)-5-(1-(tetrahydro-2H-pyran-4-yl)-1H-pyrazol-4-yl)pyridin-4-yl)piperidin-3-ol;
4-((S)-3-aminopiperidin-1-yl)-N-(6-(2-fluoro-6-methoxyphenyl)-5-nitropyridin-2-yl)-5-(1-(tetrahydro-2H-pyran-4-yl)-1H-pyrazol-4-yl)pyridin-2-amine;
methyl 6-((4-((S)-3-aminopiperidin-1-yl)-5-(1-(tetrahydro-2H-pyran-4-yl)-1H-pyrazol-4-yl)pyridin-2-yl)amino)-2-(2-fluoro-6-methoxyphenyl)nicotinate;
(S)-1-(5-(1-(difluoromethyl)-1H-pyrazol-4-yl)-2-((2-(2-fluoro-6-methoxyphenyl)pyrimidin-4-yl)amino)pyridin-4-yl)piperidin-3-ol;
(S)-N-(4-(3-aminopiperidin-1-yl)-5-(1-(difluoromethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(1-methylpiperidin-4-yl)-1H-pyrazol-4-yl)pyridin-2-yl)-2-(2-fluoro-6-methoxyphenyl)pyrimidin-4-amine;
(3S)-1-(2-((5-chloro-6-(2-fluoro-6-methoxyphenyl)pyridin-2-yl)amino)-5-(1-(tetrahydro-2H-pyran-4-yl)-1H-pyrazol-4-yl)pyridin-4-yl)piperidin-3-ol;
4-((S)-3-aminopiperidin-1-yl)-5-(1-(difluoromethyl)-1H-pyrazol-4-yl)-N-(5-fluoro-6-(2-fluoro-6-methoxyphenyl)pyridin-2-yl)pyridin-2-amine;
(R)-N-(4-(3-aminopiperidin-1-yl)-5-(1-(2,2,2-trifluoroethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(2,2,2-trifluoroethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-((tetrahydro-2H-pyran-4-yl)ethynyl)pyridin-2-yl)-2-(2-fluoro-6-methoxyphenyl)pyrimidin-4-amine;
(S)-3-(4-((4-(3-aminopiperidin-1-yl)-5-(1-(tetrahydro-2H-pyran-4-yl)-1H-pyrazol-4-yl)pyridin-2-yl)amino)pyrimidin-2-yl)pyridin-2-ol;
(S)-1-(6′-((2-(2-fluoro-6-methoxyphenyl)pyrimidin-4-yl)amino)-5-((1-methylpiperidin-4-yl)oxy)-[2,3′-bipyridin]-4′-yl)piperidin-3-ol;
(S)-4-(3-aminopiperidin-1-yl)-N-(2-(2-fluoro-6-methoxyphenyl)pyrimidin-4-yl)-6′-(tetrahydro-2H-pyran-4-yl)-[3,3′-bipyridin]-6-amine;
(S)-4-(3-aminopiperidin-1-yl)-N-(2-(2-fluoro-6-methoxyphenyl)pyrimidin-4-yl)-6′-(1-methylpiperidin-4-yl)-[3,3′-bipyridin]-6-amine;
2-(2-fluoro-6-methoxyphenyl)-N-(5-(1-(tetrahydro-2H-pyran-4-yl)-1H-pyrazol-4-yl)-4-(3-(2,2,2-trifluoroethyl)piperidin-1-yl)pyridin-2-yl)pyrimidin-4-amine;
2-(2-fluoro-6-methoxyphenyl)-N-(4-(3-(fluoromethyl)piperidin-1-yl)-5-(1-(tetrahydro-2H-pyran-4-yl)-1H-pyrazol-4-yl)pyridin-2-yl)pyrimidin-4-amine;
N-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-yl)acetamide;
2-(2-fluoro-6-methoxyphenyl)-N-(5-(1-(tetrahydro-2H-pyran-4-yl)-1H-pyrazol-4-yl)-4-(4-(trifluoromethyl)piperidin-1-yl)pyridin-2-yl)pyrimidin-4-amine;
(S)-N-(4-(3-aminopiperidin-1-yl)-5-(1-(difluoromethyl)-1H-pyrazol-4-yl)pyridin-2-yl)-2-(7-fluoroquinoxalin-6-yl)pyrimidin-4-amine;
(S)-N-(4-(3-aminopiperidin-1-yl)-5-(1-(2,2,2-trifluoroethyl)-1H-pyrazol-4-yl)pyridin-2-yl)-2-(7-fluoroquinoxalin-6-yl)pyrimidin-4-amine;
(S)-N-(4-(3-aminopiperidin-1-yl)-5-((tetrahydro-2H-pyran-4-yl)ethynyl)pyridin-2-yl)-2-(7-fluoroquinoxalin-6-yl)pyrimidin-4-amine;
(S)-N-(4-(3-aminopiperidin-1-yl)-5-(1-(2-methoxyethyl)-1H-pyrazol-4-yl)pyridin-2-yl)-2-(2-fluoro-6-methoxyphenyl)pyrimidin-4-amine;
N-(4-(2,8-diazaspiro[4.5]decan-8-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
N-(4-(2,8-diazaspiro[4.5]decan-2-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
(3R,4R)-4-fluoro-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)piperidin-3-ol;
(3R,4R)-1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)-4-(hydroxymethyl)piperidin-3-ol;
(R)-N-(4-(3-aminopyrrolidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
(S)-N-(4-(3-aminopyrrolidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(2-(1-methylpiperidin-4-yl)thiazol-5-yl)pyridin-2-yl)-2-(2-fluoro-6-methoxyphenyl)pyrimidin-4-amine;
(S)-4-(3-aminopiperidin-1-yl)-N-(2-(7-fluoroquinoxalin-6-yl)pyrimidin-4-yl)-6′-(tetrahydro-2H-pyran-4-yl)-[3,3′-bipyridin]-6-amine;
4-((S)-3-aminopiperidin-1-yl)-N-(5-fluoro-6-(2-fluoro-6-methoxyphenyl)pyridin-2-yl)-5-(1-(2,2,2-trifluoroethyl)-1H-pyrazol-4-yl)pyridin-2-amine;
(S)-(4-(4-((4-(3-aminopiperidin-1-yl)-5-(1-(difluoromethyl)-1H-pyrazol-4-yl)pyridin-2-yl)amino)pyrimidin-2-yl)-3-fluoro-5-methoxyphenyl)methanol;
(S)-(4-(4-((4-(3-aminopiperidin-1-yl)-5-(1-(2,2,2-trifluoroethyl)-1H-pyrazol-4-yl)pyridin-2-yl)amino)pyrimidin-2-yl)-3-fluoro-5-methoxyphenyl)methanol;
(S)-(4-(4-((4-(3-aminopiperidin-1-yl)-5-((tetrahydro-2H-pyran-4-yl)ethynyl)pyridin-2-yl)amino)pyrimidin-2-yl)-3-fluoro-5-methoxyphenyl)methanol;
(S)-N-(4-(3-aminopiperidin-1-yl)-5-(1-(difluoromethyl)-1H-pyrazol-4-yl)pyridin-2-yl)-2-(4-((dimethylamino)methyl)-2-fluoro-6-methoxyphenyl)pyrimidin-4-amine;
(S)-N-(4-(3-aminopiperidin-1-yl)-5-(1-(2,2,2-trifluoroethyl)-1H-pyrazol-4-yl)pyridin-2-yl)-2-(4-((dimethylamino)methyl)-2-fluoro-6-methoxyphenyl)pyrimidin-4-amine;
(S)-N-(4-(3-aminopiperidin-1-yl)-5-((tetrahydro-2H-pyran-4-yl)ethynyl)pyridin-2-yl)-2-(4-((dimethylamino)methyl)-2-fluoro-6-methoxyphenyl)pyrimidin-4-amine;
(S)-N-(4-(3-aminopiperidin-1-yl)-5-(1-(trifluoromethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(difluoromethyl)-1H-pyrazol-4-yl)pyridin-2-yl)-2-(2,4-difluoro-6-methoxyphenyl)pyrimidin-4-amine;
(S)-N-(4-(3-aminopiperidin-1-yl)-5-(1-(2,2,2-trifluoroethyl)-1H-pyrazol-4-yl)pyridin-2-yl)-2-(2,4-difluoro-6-methoxyphenyl)pyrimidin-4-amine;
(S)-N-(4-(3-aminopiperidin-1-yl)-5-((tetrahydro-2H-pyran-4-yl)ethynyl)pyridin-2-yl)-2-(2,4-difluoro-6-methoxyphenyl)pyrimidin-4-amine;
(S)-N-(4-(3-aminopiperidin-1-yl)-5-((1-(trifluoromethyl)-1H-pyrazol-4-yl)ethynyl)pyridin-2-yl)-2-(2-fluoro-6-methoxyphenyl)pyrimidin-4-amine;
2-(2-fluoro-6-methoxyphenyl)-6-((4-((S)-3-hydroxypiperidin-1-yl)-5-(1-(tetrahydro-2H-pyran-4-yl)-1H-pyrazol-4-yl)pyridin-2-yl)amino)nicotinamide;
2-(2-fluoro-6-methoxyphenyl)-6-((4-((S)-3-hydroxypiperidin-1-yl)-5-(1-(tetrahydro-2H-pyran-4-yl)-1H-pyrazol-4-yl)pyridin-2-yl)amino)-N,N-dimethylnicotinamide;
4-((S)-3-aminopiperidin-1-yl)-N-(5-fluoro-6-(2-fluoro-6-methoxyphenyl)pyridin-2-yl)-5-((1-(trifluoromethyl)-1H-pyrazol-4-yl)ethynyl)pyridin-2-amine;
(S)-N-(4-(3-aminopiperidin-1-yl)-5-((1-(trifluoromethyl)-1H-pyrazol-4-yl)ethynyl)pyridin-2-yl)-2-(7-fluoroquinoxalin-6-yl)pyrimidin-4-amine;
(S)-N-(4-(3-aminopiperidin-1-yl)-5-(1-(3-methoxypropyl)-1H-pyrazol-4-yl)pyridin-2-yl)-2-(2-fluoro-6-methoxyphenyl)pyrimidin-4-amine;
2-(2-fluoro-6-methoxyphenyl)-N-(4-(2-methyl-2,8-diazaspiro[4.5]decan-8-yl)-5-(1-(tetrahydro-2H-pyran-4-yl)-1H-pyrazol-4-yl)pyridin-2-yl)pyrimidin-4-amine;
(S)-4-(3-aminopiperidin-1-yl)-N⁶-(2-(2-fluoro-6-methoxyphenyl)pyrimidin-4-yl)-N^{6′},N^{6′}-dimethyl-[3,3′-bipyridin]-6,6′-diamine;
(S)-4-(3-aminopiperidin-1-yl)-N-(2-(2-fluoro-6-methoxyphenyl)pyrimidin-4-yl)-6′-(pyrrolidin-1-yl)-[3,3′-bipyridin]-6-amine;
(S)-4-(3-aminopiperidin-1-yl)-N-(2-(2-fluoro-6-methoxyphenyl)pyrimidin-4-yl)-6′-(piperidin-1-yl)-[3,3′-bipyridin]-6-amine;
(S)-4-(3-aminopiperidin-1-yl)-N-(2-(2-fluoro-6-methoxyphenyl)pyrimidin-4-yl)-6′-morpholino-[3,3′-bipyridin]-6-amine;
(S)-N-(5-(1-(difluoromethyl)-1H-pyrazol-4-yl)-4-(3-(methylamino)piperidin-1-yl)pyridin-2-yl)-2-(2-fluoro-6-methoxyphenyl)pyrimidin-4-amine;
(R)-N-(5-(1-(difluoromethyl)-1H-pyrazol-4-yl)-4-(3-(methylamino)piperidin-1-yl)pyridin-2-yl)-2-(2-fluoro-6-methoxyphenyl)pyrimidin-4-amine;
(S)-2-(2-fluoro-6-methoxyphenyl)-N-(4-(3-(methylamino)pyrrolidin-1-yl)-5-(1-(tetrahydro-2H-pyran-4-yl)-1H-pyrazol-4-yl)pyridin-2-yl)pyrimidin-4-amine;
(S)-N-(4-(3-(ethylamino)pyrrolidin-1-yl)-5-(1-(tetrahydro-2H-pyran-4-yl)-1H-pyrazol-4-yl)pyridin-2-yl)-2-(2-fluoro-6-methoxyphenyl)pyrimidin-4-amine;
(R)-N-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)pyrrolidin-3-yl)acetamide;
(S)-3-(6-((4-(3-aminopiperidin-1-yl)-5-(1-(tetrahydro-2H-pyran-4-yl)-1H-pyrazol-4-yl)pyridin-2-yl)amino)pyridin-2-yl)oxazolidin-2-one;
(S)-N-(1-(2-((2-(2-fluoro-6-methoxyphenyl)pyrimidin-4-yl)amino)-5-(1-(tetrahydro-2H-pyran-4-yl)-1H-pyrazol-4-yl)pyridin-4-yl)pyrrolidin-3-yl)acetamide;
6-((4-((S)-3-aminopiperidin-1-yl)-5-(1-(tetrahydro-2H-pyran-4-yl)-1H-pyrazol-4-yl)pyridin-2-yl)amino)-2-(2-fluoro-6-methoxyphenyl)nicotinamide;
6-((4-((S)-3-aminopiperidin-1-yl)-5-(1-(tetrahydro-2H-pyran-4-yl)-1H-pyrazol-4-yl)pyridin-2-yl)amino)-2-(2-fluoro-6-methoxyphenyl)-N,N-dimethylnicotinamide;
(S)-N-(4-(3-aminopiperidin-1-yl)-5-(1-methyl-3-(trifluoromethyl)-1H-pyrazol-4-yl)pyridin-2-yl)-2-(2-fluoro-6-methoxyphenyl)pyrimidin-4-amine;
(S)-2-(4-(4-(3-aminopiperidin-1-yl)-6-((2-(2-fluoro-6-methoxyphenyl)pyrimidin-4-yl)amino)pyridin-3-yl)-1H-pyrazol-1-yl)-N,N-dimethylacetamide;
(S)-2-(4-(4-(3-aminopiperidin-1-yl)-6-((2-(2-fluoro-6-methoxyphenyl)pyrimidin-4-yl)amino)pyridin-3-yl)-1H-pyrazol-1-yl)ethan-1-ol;
(S)-N-(4-(3-aminopiperidin-1-yl)-5-(1-(2,2-difluoroethyl)-1H-pyrazol-4-yl)pyridin-2-yl)-2-(2-fluoro-6-methoxyphenyl)pyrimidin-4-amine;
N-(5-(1-(difluoromethyl)-1H-pyrazol-4-yl)-4-(2-methyl-2,8-diazaspiro[4.5]decan-8-yl)pyridin-2-yl)-2-(2-fluoro-6-methoxyphenyl)pyrimidin-4-amine;
2-(2-fluoro-6-methoxyphenyl)-N-(4-(2-methyl-2,8-diazaspiro[4.5]decan-8-yl)-5-(1-(2,2,2-trifluoroethyl)-1H-pyrazol-4-yl)pyridin-2-yl)pyrimidin-4-amine;
N-(2-(2-fluoro-6-methoxyphenyl)pyrimidin-4-yl)-4-(2-methyl-2,8-diazaspiro[4.5]decan-8-yl)-6′-morpholino-[3,3′-bipyridin]-6-amine;
(S)-3-(6-((4-(3-aminopiperidin-1-yl)-6′-morpholino-[3,3′-bipyridin]-6-yl)amino)pyridin-2-yl)oxazolidin-2-one;
2-(2-fluoro-6-methoxyphenyl)-6-((4-((S)-3-hydroxypiperidin-1-yl)-5-((tetrahydro-2H-pyran-4-yl)ethynyl)pyridin-2-yl)amino)nicotinic acid;
2-(2-fluoro-6-methoxyphenyl)-6-((4-((S)-3-hydroxypiperidin-1-yl)-5-((tetrahydro-2H-pyran-4-yl)ethynyl)pyridin-2-yl)amino)nicotinamide;
6-((5-(1-(difluoromethyl)-1H-pyrazol-4-yl)-4-((S)-3-hydroxypiperidin-1-yl)pyridin-2-yl)amino)-2-(2-fluoro-6-methoxyphenyl)-N,N-dimethylnicotinamide;
6-((5-(1-(difluoromethyl)-1H-pyrazol-4-yl)-4-((S)-3-hydroxypiperidin-1-yl)pyridin-2-yl)amino)-2-(2-fluoro-6-methoxyphenyl)nicotinamide;
2-(2-fluoro-6-methoxyphenyl)-6-((4-((S)-3-hydroxypiperidin-1-yl)-5-(1-(2,2,2-trifluoroethyl)-1H-pyrazol-4-yl)pyridin-2-yl)amino)nicotinamide;
2-(2-fluoro-6-methoxyphenyl)-6-((4-((S)-3-hydroxypiperidin-1-yl)-5-(1-(2-methoxyethyl)-1H-pyrazol-4-yl)pyridin-2-yl)amino)nicotinamide;
(3S)-1-(5-(1-(difluoromethyl)-1H-pyrazol-4-yl)-2-((6-(2-fluoro-6-methoxyphenyl)-5-nitropyridin-2-yl)amino)pyridin-4-yl)piperidin-3-ol;
4-((S)-3-aminopiperidin-1-yl)-5-(1-(difluoromethyl)-1H-pyrazol-4-yl)-N-(6-(2-fluoro-6-methoxyphenyl)-5-nitropyridin-2-yl)pyridin-2-amine;
N-(5-(1-(difluoromethyl)-1H-pyrazol-4-yl)-4-(4-(2-(dimethylamino)ethyl)piperidin-1-yl)pyridin-2-yl)-2-(2-fluoro-6-methoxyphenyl)pyrimidin-4-amine;
N-(4-(4-(2-(dimethylamino)ethyl)piperidin-1-yl)-5-(1-(2,2,2-trifluoroethyl)-1H-pyrazol-4-yl)pyridin-2-yl)-2-(2-fluoro-6-methoxyphenyl)pyrimidin-4-amine;
4-(4-(2-(dimethylamino)ethyl)piperidin-1-yl)-N-(2-(2-fluoro-6-methoxyphenyl)pyrimidin-4-yl)-6′-morpholino-[3,3′-bipyridin]-6-amine;
(3S)-1-(2-((5-amino-6-(2-fluoro-6-methoxyphenyl)pyridin-2-yl)amino)-5-(1-(difluoromethyl)-1H-pyrazol-4-yl)pyridin-4-yl)piperidin-3-ol;
N²-(4-((S)-3-aminopiperidin-1-yl)-5-(1-(difluoromethyl)-1H-pyrazol-4-yl)pyridin-2-yl)-6-(2-fluoro-6-methoxyphenyl)pyridin-2,5-diamine;
4-((S)-3-aminopiperidin-1-yl)-5-(1-(2,2-difluoroethyl)-1H-pyrazol-4-yl)-N-(6-(2-fluoro-6-methoxyphenyl)-5-nitropyridin-2-yl)pyridin-2-amine;
4-((S)-3-aminopiperidin-1-yl)-N-(6-(2-fluoro-6-methoxyphenyl)-5-nitropyridin-2-yl)-5-(1-(2,2,2-trifluoroethyl)-1H-pyrazol-4-yl)pyridin-2-amine;
4-((S)-3-aminopiperidin-1-yl)-N-(6-(2-fluoro-6-methoxyphenyl)-5-nitropyridin-2-yl)-5-(1-(trifluoromethyl)-1H-pyrazol-4-yl)pyridin-2-amine;
4-((S)-3-aminopiperidin-1-yl)-N-(6-(2-fluoro-6-methoxyphenyl)-5-nitropyridin-2-yl)-5-((tetrahydro-2H-pyran-4-yl)ethynyl)pyridin-2-amine;
4-((S)-3-aminopiperidin-1-yl)-N-(6-(2-fluoro-6-methoxyphenyl)-5-nitropyridin-2-yl)-5-((1-(trifluoromethyl)-1H-pyrazol-4-yl)ethynyl)pyridin-2-amine;
(S)-N-(4-(3-aminopiperidin-1-yl)-5-(1-(trifluoromethyl)-1H-pyrazol-4-yl)pyridin-2-yl)-2-(2,4-difluoro-6-methoxyphenyl)pyrimidin-4-amine;
(S)-N-(4-(3-aminopiperidin-1-yl)-5-(1-(2,2-difluoroethyl)-1H-pyrazol-4-yl)pyridin-2-yl)-2-(2,4-difluoro-6-methoxyphenyl)pyrimidin-4-amine;
(S)-N-(4-(3-aminopiperidin-1-yl)-5-((1-(trifluoromethyl)-1H-pyrazol-4-yl)ethynyl)pyridin-2-yl)-2-(2,4-difluoro-6-methoxyphenyl)pyrimidin-4-amine;
6-((5-(1-(2,2-difluoroethyl)-1H-pyrazol-4-yl)-4-((S)-3-hydroxypiperidin-1-yl)pyridin-2-yl)amino)-2-(2-fluoro-6-methoxyphenyl)nicotinamide;
(S)-1-(2-((2-(2-fluoro-4-(hydroxymethyl)-6-methoxyphenyl)pyrimidin-4-yl)amino)-5-(1-(tetrahydro-2H-pyran-4-yl)-1H-pyrazol-4-yl)pyridin-4-yl)piperidin-3-ol;
(S)-N-(4-(3-aminopiperidin-1-yl)-5-(1-(2-morpholinoethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(2-(pyrrolidin-1-yl)ethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-((1-(difluoromethyl)-1H-pyrazol-4-yl)ethynyl)pyridin-2-yl)-2-(2-fluoro-6-methoxyphenyl)pyrimidin-4-amine;
(S)-N-(4-(3-aminopiperidin-1-yl)-5-((1-(2,2-difluoroethyl)-1H-pyrazol-4-yl)ethynyl)pyridin-2-yl)-2-(2-fluoro-6-methoxyphenyl)pyrimidin-4-amine;
(S)-N-(4-(3-aminopiperidin-1-yl)-5-((1-(2,2,2-trifluoroethyl)-1H-pyrazol-4-yl)ethynyl)pyridin-2-yl)-2-(2-fluoro-6-methoxyphenyl)pyrimidin-4-amine;
(S)-3-(4-(4-(3-aminopiperidin-1-yl)-6-((2-(2-fluoro-6-methoxyphenyl)pyrimidin-4-yl)amino)pyridin-3-yl)-1H-pyrazol-1-yl)propanamide;
(S)-N-(4-(3-aminopiperidin-1-yl)-5-(1-(azetidin-3-yl)-1H-pyrazol-4-yl)pyridin-2-yl)-2-(2-fluoro-6-methoxyphenyl)pyrimidin-4-amine;
N²-(4-((S)-3-aminopiperidin-1-yl)-5-(1-(2,2-difluoroethyl)-1H-pyrazol-4-yl)pyridin-2-yl)-6-(2-fluoro-6-methoxyphenyl)pyridin-2,5-diamine;
N²-(4-((S)-3-aminopiperidin-1-yl)-5-(1-(2,2,2-trifluoroethyl)-1H-pyrazol-4-yl)pyridin-2-yl)-6-(2-fluoro-6-methoxyphenyl)pyridin-2,5-diamine;
(S)-1-(2-((2-(2,4-difluoro-6-methoxyphenyl)pyrimidin-4-yl)amino)-5-(1-(trifluoromethyl)-1H-pyrazol-4-yl)pyridin-4-yl)piperidin-3-ol;
(S)-1-(2-((2-(2,4-difluoro-6-methoxyphenyl)pyrimidin-4-yl)amino)-5-(1-(2,2-difluoroethyl)-1H-pyrazol-4-yl)pyridin-4-yl)piperidin-3-ol;
N²-(4-((S)-3-aminopiperidin-1-yl)-5-(1-(trifluoromethyl)-1H-pyrazol-4-yl)pyridin-2-yl)-6-(2-fluoro-6-methoxyphenyl)pyridin-2,5-diamine;
(S)-1-(2-((2-(2,4-difluoro-6-methoxyphenyl)pyrimidin-4-yl)amino)-5-(1-(2,2,2-trifluoroethyl)-1H-pyrazol-4-yl)pyridin-4-yl)piperidin-3-ol;
(S)-5-(3-aminopiperidin-1-yl)-N-(2-(2-fluoro-6-methoxyphenyl)pyrimidin-4-yl)-6-(1-(2,2,2-trifluoroethyl)-1H-pyrazol-4-yl)pyridazin-3-amine;
(S)-(4-(4-(3-aminopiperidin-1-yl)-6-((2-(2-fluoro-6-methoxyphenyl)pyrimidin-4-yl)amino)pyridin-3-yl)phenyl)(4-methylpiperazin-1-yl)methanone;
(3S)-1-(2-((5-amino-6-(2-fluoro-6-methoxyphenyl)pyridin-2-yl)amino)-5-(1-(tetrahydro-2H-pyran-4-yl)-1H-pyrazol-4-yl)pyridin-4-yl)piperidin-3-ol;
(3S)-1-(2-((5-amino-6-(2-fluoro-6-methoxyphenyl)pyridin-2-yl)amino)-5-(1-(trifluoromethyl)-1H-pyrazol-4-yl)pyridin-4-yl)piperidin-3-ol;
(3S)-1-(2-((5-amino-6-(2-fluoro-6-methoxyphenyl)pyridin-2-yl)amino)-5-(1-(2,2-difluoroethyl)-1H-pyrazol-4-yl)pyridin-4-yl)piperidin-3-ol;
(3S)-1-(2-((5-amino-6-(2-fluoro-6-methoxyphenyl)pyridin-2-yl)amino)-5-(1-(2,2,2-trifluoroethyl)-1H-pyrazol-4-yl)pyridin-4-yl)piperidin-3-ol;
N²-(4-((S)-3-aminopiperidin-1-yl)-5-(1-(tetrahydro-2H-pyran-4-yl)-1H-pyrazol-4-yl)pyridin-2-yl)-6-(2-fluoro-6-methoxyphenyl)pyridin-2,5-diamine;
(S)-1-(2-((2-(2,4-difluoro-6-methoxyphenyl)pyrimidin-4-yl)amino)-5-(1-(difluoromethyl)-1H-pyrazol-4-yl)pyridin-4-yl)piperidin-3-ol;
(3S)-1-(2-((6-(2,4-difluoro-6-methoxyphenyl)-5-nitropyridin-2-yl)amino)-5-(1-(difluoromethyl)-1H-pyrazol-4-yl)pyridin-4-yl)piperidin-3-ol;
(3S)-1-(2-((5-amino-6-(2,4-difluoro-6-methoxyphenyl)pyridin-2-yl)amino)-5-(1-(difluoromethyl)-1H-pyrazol-4-yl)pyridin-4-yl)piperidin-3-ol;
methyl (S)-4-(4-((4-(3-aminopiperidin-1-yl)-5-(1-(difluoromethyl)-1H-pyrazol-4-yl)pyridin-2-yl)amino)pyrimidin-2-yl)-3-fluoro-5-methoxybenzoate;
(S)-4-(4-((4-(3-aminopiperidin-1-yl)-5-(1-(difluoromethyl)-1H-pyrazol-4-yl)pyridin-2-yl)amino)pyrimidin-2-yl)-3-fluoro-5-methoxy-N-methylbenzamide;
(S)-4-(4-((4-(3-aminopiperidin-1-yl)-5-(1-(difluoromethyl)-1H-pyrazol-4-yl)pyridin-2-yl)amino)pyrimidin-2-yl)-3-fluoro-5-methoxy-N,N-dimethylbenzamide;
(S)-1-(2-((2-(4-((3-hydroxyazetidin-1-yl)methyl)thiophen-2-yl)pyrimidin-4-yl)amino)-5-(1-(tetrahydro-2H-pyran-4-yl)-1H-pyrazol-4-yl)pyridin-4-yl)piperidin-3-ol;
(S)-3-fluoro-4-(4-((4-(3-hydroxypiperidin-1-yl)-5-(1-(tetrahydro-2H-pyran-4-yl)-1H-pyrazol-4-yl)pyridin-2-yl)amino)pyrimidin-2-yl)-5-methoxybenzaldehyde;
(S)-1-(2-((2-(2-fluoro-6-methoxy-4-((methylamino)methyl)phenyl)pyrimidin-4-yl)amino)-5-(1-(tetrahydro-2H-pyran-4-yl)-1H-pyrazol-4-yl)pyridin-4-yl)piperidin-3-ol;
6-((6-(2-fluoro-6-methoxyphenyl)-5-nitropyridin-2-yl)amino)-4-((S)-3-hydroxypiperidin-1-yl)nicotinamide;
6-((2-(2-fluoro-6-methoxyphenyl)pyrimidin-4-yl)amino)-4-((S)-3-hydroxypiperidin-1-yl)-N-((R)-1-methylpyrrolidin-3-yl)nicotinamide;
6-((5-amino-6-(2-fluoro-6-methoxyphenyl)pyridin-2-yl)amino)-4-((S)-3-hydroxypiperidin-1-yl)nicotinamide;
6-((6-(2-fluoro-6-methoxyphenyl)-5-nitropyridin-2-yl)amino)-N-((1R,3R)-3-hydroxycyclopentyl)-4-((S)-3-hydroxypiperidin-1-yl)nicotinamide;
6-((6-(2-fluoro-6-methoxyphenyl)-5-nitropyridin-2-yl)amino)-N-((1R,3S)-3-hydroxycyclopentyl)-4-((S)-3-hydroxypiperidin-1-yl)nicotinamide;
6-((6-(2-fluoro-6-methoxyphenyl)-5-nitropyridin-2-yl)amino)-N-(3-hydroxycyclobutyl)-4-((S)-3-hydroxypiperidin-1-yl)nicotinamide;
6-((6-(2-fluoro-6-methoxyphenyl)-5-nitropyridin-2-yl)amino)-N-((1R,2R)-2-hydroxycyclobutyl)-4-((S)-3-hydroxypiperidin-1-yl)nicotinamide;
N-(3,3-difluorocyclobutyl)-6-((6-(2-fluoro-6-methoxyphenyl)-5-nitropyridin-2-yl)amino)-4-((S)-3-hydroxypiperidin-1-yl)nicotinamide;
N-((1R,3R)-3-aminocyclopentyl)-6-((6-(2-fluoro-6-methoxyphenyl)-5-nitropyridin-2-yl)amino)-4-((S)-3-hydroxypiperidin-1-yl)nicotinamide;
N-((1R,3S)-3-aminocyclopentyl)-6-((6-(2-fluoro-6-methoxyphenyl)-5-nitropyridin-2-yl)amino)-4-((S)-3-hydroxypiperidin-1-yl)nicotinamide;
6-((5-amino-6-(2-fluoro-6-methoxyphenyl)pyridin-2-yl)amino)-N-((1R,3R)-3-hydroxycyclopentyl)-4-((S)-3-hydroxypiperidin-1-yl)nicotinamide;
6-((5-amino-6-(2-fluoro-6-methoxyphenyl)pyridin-2-yl)amino)-N-((1R,3S)-3-hydroxycyclopentyl)-4-((S)-3-hydroxypiperidin-1-yl)nicotinamide;
6-((6-(2-fluoro-6-methoxyphenyl)-5-nitropyridin-2-yl)amino)-4-((S)-3-hydroxypiperidin-1-yl)-N-((R)-1-methylpyrrolidin-3-yl)nicotinamide;
N-(4-((3S,4R)-3-amino-4-methylpiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3S,5S)-3-amino-5-methylpiperidin-1-yl)-5-(1-(difluoromethyl)-1H-pyrazol-4-yl)pyridin-2-yl)-2-(2-fluoro-6-methoxyphenyl)pyrimidin-4-amine;
(S)-N-(4-(3-amino-3-methylpiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3R,4S)-3-amino-4-fluoropiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3R,4R)-3-amino-4-fluoropiperidin-1-yl)-5-(1-(difluoromethyl)-1H-pyrazol-4-yl)pyridin-2-yl)-2-(2-fluoro-6-methoxyphenyl)pyrimidin-4-amine;
(3R,4R)-3-amino-1-(5-(1-(difluoromethyl)-1H-pyrazol-4-yl)-2-((2-(2-fluoro-6-methoxyphenyl)pyrimidin-4-yl)amino)pyridin-4-yl)piperidin-4-ol;
N-(4-((3S,5S)-3-amino-5-methylpiperidin-1-yl)-5-(1-(difluoromethyl)-1H-pyrazol-4-yl)pyridin-2-yl)-2-(2,4-difluoro-6-methoxyphenyl)pyrimidin-4-amine;
N-(4-((3S,4R)-3-amino-4-methylpiperidin-1-yl)-5-(1-(difluoromethyl)-1H-pyrazol-4-yl)pyridin-2-yl)-2-(2,4-difluoro-6-methoxyphenyl)pyrimidin-4-amine;
(S)-N-(4-(3-amino-3-methylpiperidin-1-yl)-5-(1-(difluoromethyl)-1H-pyrazol-4-yl)pyridin-2-yl)-2-(2,4-difluoro-6-methoxyphenyl)pyrimidin-4-amine;
(3R,4R)-3-amino-1-(2-((2-(2,4-difluoro-6-methoxyphenyl)pyrimidin-4-yl)amino)-5-(1-(difluoromethyl)-1H-pyrazol-4-yl)pyridin-4-yl)piperidin-4-ol;
(1S,4S)-4-((5-(1-(difluoromethyl)-1H-pyrazol-4-yl)-2-((2-(2-fluoro-6-methoxyphenyl)pyrimidin-4-yl)amino)pyridin-4-yl)(methyl)amino)cyclohexan-1-ol;
N²-(4-((3S,4R)-3-amino-4-methylpiperidin-1-yl)-5-(1-(difluoromethyl)-1H-pyrazol-4-yl)pyridin-2-yl)-6-(2,4-difluoro-6-methoxyphenyl)pyridin-2,5-diamine;
N²-(4-((3R,4R)-3-amino-4-fluoropiperidin-1-yl)-5-(1-(difluoromethyl)-1H-pyrazol-4-yl)pyridin-2-yl)-6-(2,4-difluoro-6-methoxyphenyl)pyridin-2,5-diamine;
(S)-1-(2-((2-(2,4-difluoro-6-methoxyphenyl)pyrimidin-4-yl)amino)-5-((1-(difluoromethyl)-1H-pyrazol-4-yl)ethynyl)pyridin-4-yl)piperidin-3-ol;
(S)-1-(2-((2-(2,4-difluoro-6-methoxyphenyl)pyrimidin-4-yl)amino)-5-((1-(trifluoromethyl)-1H-pyrazol-4-yl)ethynyl)pyridin-4-yl)piperidin-3-ol;
(S)-1-(2-((2-(2,4-difluoro-6-methoxyphenyl)pyrimidin-4-yl)amino)-5-((1-(2,2-difluoroethyl)-1H-pyrazol-4-yl)ethynyl)pyridin-4-yl)piperidin-3-ol;
(S)-1-(2-((2-(2,4-difluoro-6-methoxyphenyl)pyrimidin-4-yl)amino)-5-((1-(2,2,2-trifluoroethyl)-1H-pyrazol-4-yl)ethynyl)pyridin-4-yl)piperidin-3-ol;
(3S)-1-(5-((1-(2,2-difluoroethyl)-1H-pyrazol-4-yl)ethynyl)-2-((6-(2-fluoro-6-methoxyphenyl)-5-nitropyridin-2-yl)amino)pyridin-4-yl)piperidin-3-ol;
(3S)-1-(5-((1-(difluoromethyl)-1H-pyrazol-4-yl)ethynyl)-2-((6-(2-fluoro-6-methoxyphenyl)-5-nitropyridin-2-yl)amino)pyridin-4-yl)piperidin-3-ol;
(3S)-1-(2-((6-(2,4-difluoro-6-methoxyphenyl)-5-nitropyridin-2-yl)amino)-5-(1-(2,2-difluoroethyl)-1H-pyrazol-4-yl)pyridin-4-yl)piperidin-3-ol;
(3S)-1-(2-((6-(2,4-difluoro-6-methoxyphenyl)-5-nitropyridin-2-yl)amino)-5-(1-(tetrahydro-2H-pyran-4-yl)-1H-pyrazol-4-yl)pyridin-4-yl)piperidin-3-ol;
(3S)-1-(2-((6-(2,4-difluoro-6-methoxyphenyl)-5-nitropyridin-2-yl)amino)-5-((tetrahydro-2H-pyran-4-yl)ethynyl)pyridin-4-yl)piperidin-3-ol;
(3S)-1-(2-((5-amino-6-(2,4-difluoro-6-methoxyphenyl)pyridin-2-yl)amino)-5-(1-(2,2-difluoroethyl)-1H-pyrazol-4-yl)pyridin-4-yl)piperidin-3-ol;
(3S)-1-(2-((5-amino-6-(2,4-difluoro-6-methoxyphenyl)pyridin-2-yl)amino)-5-(1-(tetrahydro-2H-pyran-4-yl)-1H-pyrazol-4-yl)pyridin-4-yl)piperidin-3-ol;
(3S)-1-(2-((5-amino-6-(2,4-difluoro-6-methoxyphenyl)pyridin-2-yl)amino)-5-(2-(tetrahydro-2H-pyran-4-yl)ethyl)pyridin-4-yl)piperidin-3-ol;
(S)-N-(4-(3-aminopiperidin-1-yl)-5-(1-(2-methoxy-2-methylpropyl)-1H-pyrazol-4-yl)pyridin-2-yl)-2-(2-fluoro-6-methoxyphenyl)pyrimidin-4-amine;
(S)-4-(3-aminopiperidin-1-yl)-N-(2-(2-fluoro-6-methoxyphenyl)pyrimidin-4-yl)-6′-(2-methoxyethoxy)-[3,3′-bipyridin]-6-amine;
(S)-(3,3-difluoroazetidin-1-yl)(4-(4-((5-(1-(difluoromethyl)-1H-pyrazol-4-yl)-4-(3-hydroxypiperidin-1-yl)pyridin-2-yl)amino)pyrimidin-2-yl)-3-fluoro-5-methoxyphenyl)methanone;
(4-(4-((5-(1-(difluoromethyl)-1H-pyrazol-4-yl)-4-((S)-3-hydroxypiperidin-1-yl)pyridin-2-yl)amino)pyrimidin-2-yl)-3-fluoro-5-methoxyphenyl)((S)-3-fluoropyrrolidin-1-yl)methanone;
(4-(4-((5-(1-(difluoromethyl)-1H-pyrazol-4-yl)-4-((S)-3-hydroxypiperidin-1-yl)pyridin-2-yl)amino)pyrimidin-2-yl)-3-fluoro-5-methoxyphenyl)((R)-3-fluoropyrrolidin-1-yl)methanone;
(S)-(4-(4-((5-(1-(difluoromethyl)-1H-pyrazol-4-yl)-4-(3-hydroxypiperidin-1-yl)pyridin-2-yl)amino)pyrimidin-2-yl)-3-fluoro-5-methoxyphenyl)(3,3-difluoropyrrolidin-1-yl)methanone;
(S)-1-(4-(4-(3-aminopiperidin-1-yl)-6-((2-(2-fluoro-6-methoxyphenyl)pyrimidin-4-yl)amino)pyridin-3-yl)-1H-pyrazol-1-yl)-2-methylpropan-2-ol;
(S)-N-(4-(3-aminopiperidin-1-yl)-5-(1-(3-(dimethylamino)propyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(azetidin-3-ylmethyl)-1H-pyrazol-4-yl)pyridin-2-yl)-2-(2-fluoro-6-methoxyphenyl)pyrimidin-4-amine;
N-(4-((S)-3-aminopiperidin-1-yl)-5-(1-((R)-pyrrolidin-3-yl)-1H-pyrazol-4-yl)pyridin-2-yl)-2-(2-fluoro-6-methoxyphenyl)pyrimidin-4-amine;
N-(4-((S)-3-aminopiperidin-1-yl)-5-(1-((S)-pyrrolidin-3-yl)-1H-pyrazol-4-yl)pyridin-2-yl)-2-(2-fluoro-6-methoxyphenyl)pyrimidin-4-amine;
(S)-4-(3-aminopiperidin-1-yl)-N^{6′}-(2-(dimethylamino)ethyl)-N⁶-(2-(2-fluoro-6-methoxyphenyl)pyrimidin-4-yl)-[3,3′-bipyridin]-6,6′-diamine;
(S)-4-(3-aminopiperidin-1-yl)-N-(2-(2-fluoro-6-methoxyphenyl)pyrimidin-4-yl)-6′-(piperazin-1-yl)-[3,3′-bipyridin]-6-amine;
(S)-(2-(4-((4-(3-aminopiperidin-1-yl)-5-(1-(difluoromethyl)-1H-pyrazol-4-yl)pyridin-2-yl)amino)pyrimidin-2-yl)-3-fluorophenyl)methanol;
(S)-(2-(4-((4-(3-aminopiperidin-1-yl)-5-((1-(difluoromethyl)-1H-pyrazol-4-yl)ethynyl)pyridin-2-yl)amino)pyrimidin-2-yl)-3-fluorophenyl)methanol;
(S)-N-(4-(3-aminopiperidin-1-yl)-5-(1-(1-methoxy-2-methylpropan-2-yl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-((3,3-difluorocyclobutyl)methyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(2-(dimethylamino)ethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(fluoromethyl)-1H-pyrazol-4-yl)pyridin-2-yl)-2-(2-fluoro-6-methoxyphenyl)pyrimidin-4-amine;
(S)-4-(3-aminopiperidin-1-yl)-N-(2-(2-fluoro-6-methoxyphenyl)pyrimidin-4-yl)-6′-(piperidin-4-yl)-[3,3′-bipyridin]-6-amine;
(S)-4-(3-aminopiperidin-1-yl)-6′-(2-(dimethylamino)ethoxy)-N-(2-(2-fluoro-6-methoxyphenyl)pyrimidin-4-yl)-[3,3′-bipyridin]-6-amine;
(S)-1-(4′-(3-aminopiperidin-1-yl)-6′-((2-(2-fluoro-6-methoxyphenyl)pyrimidin-4-yl)amino)-[3,3′-bipyridin]-6-yl)-3-ethylurea;
(4-(4-((4-((S)-3-aminopiperidin-1-yl)-5-(1-(difluoromethyl)-1H-pyrazol-4-yl)pyridin-2-yl)amino)pyrimidin-2-yl)-3-fluoro-5-methoxyphenyl)((R)-3-fluoropyrrolidin-1-yl)methanone;
N-(4-((2R,5S)-5-amino-2-methylpiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3R,4S)-3-amino-4-methoxypiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3R,4R)-3-amino-4-methoxypiperidin-1-yl)-5-(1-(difluoromethyl)-1H-pyrazol-4-yl)pyridin-2-yl)-2-(2-fluoro-6-methoxyphenyl)pyrimidin-4-amine;
(S)-(4-(4-(3-aminopiperidin-1-yl)-6-((2-(2-fluoro-6-methoxyphenyl)pyrimidin-4-yl)amino)pyridin-3-yl)phenyl)(morpholino)methanone;
(S)-N-(4-(3-aminopiperidin-1-yl)-5-(4-methyl-3,4-dihydro-2H-pyrido[3,2-b][1,4]oxazin-7-yl)pyridin-2-yl)-2-(2-fluoro-6-methoxyphenyl)pyrimidin-4-amine;
(S)-N-(4-(3-aminopiperidin-1-yl)-5-(4-methyl-3,4-dihydro-2H-benzo[b][1,4]oxazin-7-yl)pyridin-2-yl)-2-(2-fluoro-6-methoxyphenyl)pyrimidin-4-amine;
(S)-6-(4-(3-aminopiperidin-1-yl)-6-((2-(2-fluoro-6-methoxyphenyl)pyrimidin-4-yl)amino)pyridin-3-yl)-1,4-dihydro-2H-benzo[d][1,3]oxazin-2-one;
(S)-1-(4-(4-(3-aminopiperidin-1-yl)-6-((2-(2-fluoro-6-methoxyphenyl)pyrimidin-4-yl)amino)pyridin-3-yl)phenyl)pyrrolidin-2-one;
(S)-7-(4-(3-aminopiperidin-1-yl)-6-((2-(2-fluoro-6-methoxyphenyl)pyrimidin-4-yl)amino)pyridin-3-yl)-2H-benzo[b][1,4]oxazin-3(4H)-one;
(S)-7-(4-(3-aminopiperidin-1-yl)-6-((2-(2-fluoro-6-methoxyphenyl)pyrimidin-4-yl)amino)pyridin-3-yl)-4-methyl-2H-benzo[b][1,4]oxazin-3(4H)-one;
(S)-6-(4-(3-aminopiperidin-1-yl)-6-((2-(2-fluoro-6-methoxyphenyl)pyrimidin-4-yl)amino)pyridin-3-yl)-8-methyl-3,4-dihydroquinolin-2(1H)-one;
(S)-6-(4-(3-aminopiperidin-1-yl)-6-((2-(2-fluoro-6-methoxyphenyl)pyrimidin-4-yl)amino)pyridin-3-yl)-1-methyl-3,4-dihydroquinolin-2(1H)-one;
(S)-1-(2-((2-(2-fluoro-6-methoxyphenyl)pyrimidin-4-yl)amino)-5-(1-(2,2,6,6-tetramethyltetrahydro-2H-pyran-4-yl)-1H-pyrazol-4-yl)pyridin-4-yl)piperidin-3-ol;
(S)-(4-(4-(3-aminopiperidin-1-yl)-6-((2-(2-fluoro-6-methoxyphenyl)pyrimidin-4-yl)amino)pyridin-3-yl)phenyl)(piperazin-1-yl)methanone;
(S)-N-(5-(1-(difluoromethyl)-1H-pyrazol-4-yl)-4-(3-((methylamino)methyl)piperidin-1-yl)pyridin-2-yl)-2-(2-fluoro-6-methoxyphenyl)pyrimidin-4-amine;
(R)-N-(5-(1-(difluoromethyl)-1H-pyrazol-4-yl)-4-(3-((methylamino)methyl)piperidin-1-yl)pyridin-2-yl)-2-(2-fluoro-6-methoxyphenyl)pyrimidin-4-amine;
N-(4-((3R,4R)-4-amino-3-methylpiperidin-1-yl)-5-(1-(difluoromethyl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(1-(2,2,6,6-tetramethyltetrahydro-2H-pyran-4-yl)-1H-pyrazol-4-yl)pyridin-2-yl)-2-(2-fluoro-6-methoxyphenyl)pyrimidin-4-amine;
6-(4-((S)-3-aminopiperidin-1-yl)-6-((2-(2-fluoro-6-methoxyphenyl)pyrimidin-4-yl)amino)pyridin-3-yl)-2-methyl-2H-benzo[b][1,4]oxazin-3(4H)-one;
(S)-N-(5-(1-(difluoromethyl)-1H-pyrazol-4-yl)-4-(3-methylpiperidin-1-yl)pyridin-2-yl)-2-(2-fluoro-6-methoxyphenyl)pyrimidin-4-amine;
(S)-N-(5-(1-(difluoromethyl)-1H-pyrazol-4-yl)-4-(3-(fluoromethyl)piperidin-1-yl)pyridin-2-yl)-2-(2-fluoro-6-methoxyphenyl)pyrimidin-4-amine;
(R)-N-(5-(1-(difluoromethyl)-1H-pyrazol-4-yl)-4-(3-(difluoromethyl)piperidin-1-yl)pyridin-2-yl)-2-(2-fluoro-6-methoxyphenyl)pyrimidin-4-amine;
(S)-7-(4-(3-aminopiperidin-1-yl)-6-((2-(2-fluoro-6-methoxyphenyl)pyrimidin-4-yl)amino)pyridin-3-yl)-4-methyl-2H-pyrido[3,2-b][1,4]oxazin-3(4H)-one;
(S)-7-(4-(3-aminopiperidin-1-yl)-6-((2-(2-fluoro-6-methoxyphenyl)pyrimidin-4-yl)amino)pyridin-3-yl)-2H-pyrido[3,2-b][1,4]oxazin-3(4H)-one;
(S)-6-(4-(3-aminopiperidin-1-yl)-6-((2-(2-fluoro-6-methoxyphenyl)pyrimidin-4-yl)amino)pyridin-3-yl)-3,4-dihydro-1,8-naphthyridin-2(1H)-one;
(S)-N-(4-(3-aminopiperidin-1-yl)-5-(1-(oxetan-3-yl)-1H-pyrazol-4-yl)pyridin-2-yl)-2-(2-fluoro-6-methoxyphenyl)pyrimidin-4-amine;
methyl (S)-2-(4-(4-(3-aminopiperidin-1-yl)-6-((2-(2-fluoro-6-methoxyphenyl)pyrimidin-4-yl)amino)pyridin-3-yl)-1H-pyrazol-1-yl)-2-methylpropanoate;
(S)-N-(4-(4-(3-aminopiperidin-1-yl)-6-((2-(2-fluoro-6-methoxyphenyl)pyrimidin-4-yl)amino)pyridin-3-yl)phenyl)acetamide;
(S)-3-(4-(3-aminopiperidin-1-yl)-6-((2-(2-fluoro-6-methoxyphenyl)pyrimidin-4-yl)amino)pyridin-3-yl)-N,N-dimethylbenzamide;
(S)-N-(4-(3-aminopiperidin-1-yl)-5-(3-methyl-4-(trifluoromethoxy)phenyl)pyridin-2-yl)-2-(2-fluoro-6-methoxyphenyl)pyrimidin-4-amine;
(S)-N-(4-(3-aminopiperidin-1-yl)-5-(4-fluoro-3-methylphenyl)pyridin-2-yl)-2-(2-fluoro-6-methoxyphenyl)pyrimidin-4-amine;
(S)-N-(4-(3-aminopiperidin-1-yl)-5-(1-(piperidin-4-yl)-1H-pyrazol-4-yl)pyridin-2-yl)-2-(2-fluoro-6-methoxyphenyl)pyrimidin-4-amine;
(S)-N-(4-(3-aminopiperidin-1-yl)-5-(4-isopropoxy-3,5-dimethylphenyl)pyridin-2-yl)-2-(2-fluoro-6-methoxyphenyl)pyrimidin-4-amine;
(S)-N-(4-(3-aminopiperidin-1-yl)-5-(4-methoxy-3,5-dimethylphenyl)pyridin-2-yl)-2-(2-fluoro-6-methoxyphenyl)pyrimidin-4-amine;
(S)-N-(4-(3-aminopiperidin-1-yl)-5-(4-morpholinophenyl)pyridin-2-yl)-2-(2-fluoro-6-methoxyphenyl)pyrimidin-4-amine;
(S)-N-(4-(3-aminopiperidin-1-yl)-5-(3-methyl-4-morpholinophenyl)pyridin-2-yl)-2-(2-fluoro-6-methoxyphenyl)pyrimidin-4-amine;
(S)-(4-(4-(3-aminopiperidin-1-yl)-6-((2-(2-fluoro-6-methoxyphenyl)pyrimidin-4-yl)amino)pyridin-3-yl)phenyl)(4-(2-hydroxyethyl)piperazin-1-yl)methanone;
4-(4-((S)-3-aminopiperidin-1-yl)-6-((2-(2-fluoro-6-methoxyphenyl)pyrimidin-4-yl)amino)pyridin-3-yl)-N-((1r,4S)-4-hydroxycyclohexyl)benzamide;
(S)-4-(4-(3-aminopiperidin-1-yl)-6-((2-(2-fluoro-6-methoxyphenyl)pyrimidin-4-yl)amino)pyridin-3-yl)-2-fluoro-N,N-dimethylbenzamide;
(S)-1-(3-(4-(3-aminopiperidin-1-yl)-6-((2-(2-fluoro-6-methoxyphenyl)pyrimidin-4-yl)amino)pyridin-3-yl)phenyl)ethan-1-one;
(S)-N-(4-(3-aminopiperidin-1-yl)-5-(3-chloro-4-fluorophenyl)pyridin-2-yl)-2-(2-fluoro-6-methoxyphenyl)pyrimidin-4-amine;
(S)-N-(4-(3-aminopiperidin-1-yl)-5-(4-fluoro-3-(trifluoromethyl)phenyl)pyridin-2-yl)-2-(2-fluoro-6-methoxyphenyl)pyrimidin-4-amine;
(S)-N-(4-(3-aminopiperidin-1-yl)-5-(3,4-difluorophenyl)pyridin-2-yl)-2-(2-fluoro-6-methoxyphenyl)pyrimidin-4-amine;
(3R,4R)-4-amino-1-(5-(1-(difluoromethyl)-1H-pyrazol-4-yl)-2-((2-(2-fluoro-6-methoxyphenyl)pyrimidin-4-yl)amino)pyridin-4-yl)piperidin-3-ol;
(S)-N-(4-(3-aminopiperidin-1-yl)-5-(3-(dimethylamino)-4-fluorophenyl)pyridin-2-yl)-2-(2-fluoro-6-methoxyphenyl)pyrimidin-4-amine;
N-(4-((3R,4S)-4-amino-3-methylpiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((S)-3-aminopiperidin-1-yl)-5-(1-((S)-tetrahydrofuran-3-yl)-1H-pyrazol-4-yl)pyridin-2-yl)-2-(2-fluoro-6-methoxyphenyl)pyrimidin-4-amine;
N-(4-((S)-3-aminopiperidin-1-yl)-5-(1-((R)-tetrahydrofuran-3-yl)-1H-pyrazol-4-yl)pyridin-2-yl)-2-(2-fluoro-6-methoxyphenyl)pyrimidin-4-amine;
6-((4-((S)-3-aminopiperidin-1-yl)-5-(1-(tetrahydro-2H-pyran-4-yl)-1H-pyrazol-4-yl)pyridin-2-yl)amino)-2-(2-fluoro-6-methoxyphenyl)nicotinonitrile;
6-((4-((S)-3-aminopiperidin-1-yl)-5-(1-(difluoromethyl)-1H-pyrazol-4-yl)pyridin-2-yl)amino)-2-(2-fluoro-6-methoxyphenyl)nicotinonitrile;
N-(4-((3S,4S)-4-amino-3-fluoropiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3S,5R)-3-amino-5-fluoropiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3S,5S)-3-amino-5-fluoropiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3S,5R)-3-amino-5-(trifluoromethyl)piperidin-1-yl)-5-(1-(difluoromethyl)-1H-pyrazol-4-yl)pyridin-2-yl)-2-(2-fluoro-6-methoxyphenyl)pyrimidin-4-amine;
(3S,5S)-5-amino-1-(5-(1-(difluoromethyl)-1H-pyrazol-4-yl)-2-((2-(2-fluoro-6-methoxyphenyl)pyrimidin-4-yl)amino)pyridin-4-yl)piperidin-3-ol;
N-(4-((3R,4S)-4-amino-3-fluoropiperidin-1-yl)-5-(1-(difluoromethyl)-1H-pyrazol-4-yl)pyridin-2-yl)-2-(2-fluoro-6-methoxyphenyl)pyrimidin-4-amine;
N-(4-((3S,4R)-4-amino-3-fluoropiperidin-1-yl)-5-(1-(difluoromethyl)-1H-pyrazol-4-yl)pyridin-2-yl)-2-(2-fluoro-6-methoxyphenyl)pyrimidin-4-amine;
6-((4-((S)-3-aminopiperidin-1-yl)-5-(1-(tetrahydro-2H-pyran-4-yl)-1H-pyrazol-4-yl)pyridin-2-yl)amino)-2-(2,4-difluoro-6-methoxyphenyl)nicotinonitrile;
6-((4-((S)-3-aminopiperidin-1-yl)-5-(1-(difluoromethyl)-1H-pyrazol-4-yl)pyridin-2-yl)amino)-2-(2,4-difluoro-6-methoxyphenyl)nicotinonitrile;
(S)-N-(4-(3-aminopiperidin-1-yl)-5-((4-methyltetrahydro-2H-pyran-4-yl)ethynyl)pyridin-2-yl)-2-(2,4-difluoro-6-methoxyphenyl)pyrimidin-4-amine;
(S)-4-((4-(3-aminopiperidin-1-yl)-6-((2-(2,4-difluoro-6-methoxyphenyl)pyrimidin-4-yl)amino)pyridin-3-yl)ethynyl)tetrahydro-2H-pyran-4-ol;
(S)-4-(4-(3-aminopiperidin-1-yl)-6-((2-(2,4-difluoro-6-methoxyphenyl)pyrimidin-4-yl)amino)pyridin-3-yl)-2-methylbut-3-yn-2-ol;
(S)-4-(4-(3-aminopiperidin-1-yl)-6-((2-(2,4-difluoro-6-methoxyphenyl)pyrimidin-4-yl)amino)pyridin-3-yl)but-3-yn-1-ol;
4-((S)-3-aminopiperidin-1-yl)-5-(1-(difluoromethyl)-1H-pyrazol-4-yl)-N-(6-(2-fluoro-6-methoxyphenyl)-5-(methylsulfonyl)pyridin-2-yl)pyridin-2-amine;
2-(2-fluoro-6-methoxyphenyl)-6-((4-((S)-3-hydroxypiperidin-1-yl)-5-(1-(2,2,2-trifluoroethyl)-1H-pyrazol-4-yl)pyridin-2-yl)amino)nicotinonitrile;
2-(2,4-difluoro-6-methoxyphenyl)-6-((4-((S)-3-hydroxypiperidin-1-yl)-5-(1-(2,2,2-trifluoroethyl)-1H-pyrazol-4-yl)pyridin-2-yl)amino)nicotinonitrile;
6-((4-((S)-3-aminopiperidin-1-yl)-5-(1-(2,2,2-trifluoroethyl)-1H-pyrazol-4-yl)pyridin-2-yl)amino)-2-(2-fluoro-6-methoxyphenyl)nicotinonitrile;
6-((4-((S)-3-aminopiperidin-1-yl)-5-(1-(2-hydroxy-2-methylpropyl)-1H-pyrazol-4-yl)pyridin-2-yl)amino)-2-(2-fluoro-6-methoxyphenyl)nicotinonitrile;
6-((4-((S)-3-aminopiperidin-1-yl)-5-(1-(2,2,2-trifluoroethyl)-1H-pyrazol-4-yl)pyridin-2-yl)amino)-2-(2,4-difluoro-6-methoxyphenyl)nicotinonitrile;
6-((4-((3R,4R)-3-amino-4-fluoropiperidin-1-yl)-5-(1-(difluoromethyl)-1H-pyrazol-4-yl)pyridin-2-yl)amino)-2-(2-fluoro-6-methoxyphenyl)nicotinonitrile;
6-((4-((3R,4S)-3-amino-4-fluoropiperidin-1-yl)-5-(1-(difluoromethyl)-1H-pyrazol-4-yl)pyridin-2-yl)amino)-2-(2-fluoro-6-methoxyphenyl)nicotinonitrile;
6-((4-((S)-3-aminopiperidin-1-yl)-6′-morpholino-[3,3′-bipyridin]-6-yl)amino)-2-(2-fluoro-6-methoxyphenyl)nicotinonitrile;
N-(4-((3S,5R)-3-amino-5-fluoropiperidin-1-yl)-5-(2-(2,2,2-trifluoroethyl)-1H-pyrazol-4-yl)pyridin-2-yl)-2-(2,4-difluoro-6-methoxyphenyl)pyrimidin-4-amine;
2-(2-fluoro-6-methoxyphenyl)-6-((4-((S)-3-hydroxypiperidin-1-yl)-5-(1-(1-methylpiperidin-4-yl)-1H-pyrazol-4-yl)pyridin-2-yl)amino)nicotinonitrile;
6-((4-((3R,4R)-4-amino-3-hydroxypiperidin-1-yl)-5-(1-(difluoromethyl)-1H-pyrazol-4-yl)pyridin-2-yl)amino)-2-(2-fluoro-6-methoxyphenyl)nicotinonitrile;
6-((4-((S)-3-aminopiperidin-1-yl)-5-(1-ethyl-1H-pyrazol-4-yl)pyridin-2-yl)amino)-2-(2-fluoro-6-methoxyphenyl)nicotinonitrile;
6-((4-((S)-3-aminopiperidin-1-yl)-5-(1-isopropyl-1H-pyrazol-4-yl)pyridin-2-yl)amino)-2-(2-fluoro-6-methoxyphenyl)nicotinonitrile;
6-((4-((S)-3-aminopiperidin-1-yl)-5-(1-cyclopropyl-1H-pyrazol-4-yl)pyridin-2-yl)amino)-2-(2-fluoro-6-methoxyphenyl)nicotinonitrile;
6-((4-((S)-3-aminopiperidin-1-yl)-5-(1-(cyclopropylmethyl)-1H-pyrazol-4-yl)pyridin-2-yl)amino)-2-(2-fluoro-6-methoxyphenyl)nicotinonitrile;
6-((5-(1-(difluoromethyl)-1H-pyrazol-4-yl)-4-((3R,4R)-4-fluoro-3-hydroxypiperidin-1-yl)pyridin-2-yl)amino)-2-(2-fluoro-6-methoxyphenyl)nicotinonitrile;
N-(4-((3S,5S)-3-amino-5-fluoropiperidin-1-yl)-5-(1-(2,2,2-trifluoroethyl)-1H-pyrazol-4-yl)pyridin-2-yl)-2-(2-fluoro-6-methoxyphenyl)pyrimidin-4-amine;
N-(4-((3S,5S)-3-amino-5-fluoropiperidin-1-yl)-5-(1-(2,2,2-trifluoroethyl)-1H-pyrazol-4-yl)pyridin-2-yl)-2-(2,4-difluoro-6-methoxyphenyl)pyrimidin-4-amine;
4-((3R,4R)-3-amino-4-fluoropiperidin-1-yl)-5-(1-(difluoromethyl)-1H-pyrazol-4-yl)-N-(6-(2-fluoro-6-methoxyphenyl)-5-nitropyridin-2-yl)pyridin-2-amine;
N-(4-((3S,5R)-3-amino-5-fluoropiperidin-1-yl)-5-(1-isopropyl-1H-pyrazol-4-yl)pyridin-2-yl)-2-(2,4-difluoro-6-methoxyphenyl)pyrimidin-4-amine;
N-(4-((3S,5S)-3-amino-5-fluoropiperidin-1-yl)-5-(1-isopropyl-1H-pyrazol-4-yl)pyridin-2-yl)-2-(2,4-difluoro-6-methoxyphenyl)pyrimidin-4-amine;
6-((4-((3S,4R)-3-amino-4-methylpiperidin-1-yl)-5-(1-(difluoromethyl)-1H-pyrazol-4-yl)pyridin-2-yl)amino)-2-(2-fluoro-6-methoxyphenyl)-nicotinonitrile;
6-((4-((3S,4R)-3-amino-4-methylpiperidin-1-yl)-5-(1-(difluoromethyl)-1H-pyrazol-4-yl)pyridin-2-yl)amino)-2-(2,4-difluoro-6-methoxyphenyl)nicotinonitrile;
N-(4-((3R,4R)-3-amino-4-fluoropiperidin-1-yl)-5-(1-isopropyl-1H-pyrazol-4-yl)pyridin-2-yl)-2-(2,4-difluoro-6-methoxyphenyl)pyrimidin-4-amine;
6-((4-((3R,4R)-3-amino-4-fluoropiperidin-1-yl)-5-(1-isopropyl-1H-pyrazol-4-yl)pyridin-2-yl)amino)-2-(2,4-difluoro-6-methoxyphenyl)nicotinonitrile; and
4-((3R,4R)-3-amino-4-fluoropiperidin-1-yl)-5-(1-(difluoromethyl)-1H-pyrazol-4-yl)-N-(5-fluoro-6-(2-fluoro-6-methoxyphenyl)pyridin-2-yl)pyridin-2-amine.

25. A pharmaceutical composition for inhibiting hematopoietic progenitor kinase 1 comprising the compound or pharmaceutically acceptable salt thereof as claimed in claim 15 as an active ingredient.

26. The pharmaceutical composition as claimed in claim 25, for preventing or treating a blood cancer or a solid tumor.

27. The pharmaceutical composition as claimed in claim 25, for preventing or treating melanoma, blastoma, sarcoma, lymphoma, leukemia, bladder cancer, brain tumor, breast cancer, cervical cancer, colorectal cancer, esophageal cancer, endometrioma, hepatic cancer, laryngeal cancer, lung cancer, osteosarcoma, ovarian cancer, pancreatic cancer, prostate cancer, renal cancer, thyroid cancer, ependymoma, glioblastoma, bullous cell tumor, neuroblastoma, osteosarcoma, rhabdomyosarcoma, or nephroblastoma.

28. A method for inhibiting hematopoietic progenitor kinase 1 in a mammal, which comprises administering a therapeutically effective amount of the compound or pharmaceutically acceptable salt thereof as claimed in claim 15 to the mammal in need thereof.
